(19) United States
(12) Patent Application Publication
(10) Pub. No.: US 2024/0392004 A1
(43) Pub. Date: Nov. 28, 2024

(54) IMMUNOMODULATORY MOLECULES AND USES THEREOF

(71) Applicant: Immunowake Inc., Birmingham, AL (US)

(72) Inventors: Ellen WU, Birmingham, AL (US); Xiaoyun WU, Birmingham, AL (US); John WAKEFIELD, Birmingham, AL (US)

(21) Appl. No.: 18/281,216

(22) PCT Filed: Mar. 10, 2022
(86) PCT No.: PCT/US2022/071077

§ 371 Date: Sep. 8, 2023

(51) Int. Cl. C07K 16/28; A61K 39/00; A61P 35/00; C07K 14/54; C07K 14/55; C07K 14/56; C07K 14/57

(52) U.S. Cl. CPC C07K 16/2818; A61P 35/00; C07K 14/54; C07K 14/5428; C07K 14/5434; C07K 14/55; C07K 14/56; C07K 14/57; C07K 16/2827; A61K 2039/505; C07K 2317/53; C07K 2317/55; C07K 2317/76

(57) ABSTRACT

The present application relates to immunomodulatory molecules comprising a first binding domain (e.g., immunostimulatory cytokine such as IL-2 or IL-12 or variant thereof) specifically recognizing a first target molecule (e.g., receptor of immunostimulatory cytokine) and a second binding domain (e.g., agonist ligand such as PD-L1 or PD-L2 or variant thereof, or agonist antigen-binding fragment such as anti-PD-1 agonist Fab, scFv, VHH, or full-length antibody) specifically recognizing a second target molecule (e.g., inhibitory checkpoint molecule such as PD-1), wherein the first binding domain upon binding to the first target molecule up-regulates an immune response, and wherein the second, binding domain upon binding to the second target molecule down-regulates the immune response. Methods of making and uses of such immunomodulatory molecules are also provided.

### CROSS REFERENCE TO RELATED APPLICATIONS

This application claims priority benefits of U.S. Provisional Patent Application No. 63/159,441 filed Mar. 10, 2021, and International Patent Application No. PCT/US2021/073107 filed Dec. 23, 2021, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 754392000740SEQLIST.TXT, date recorded: Mar. 10, 2022, size: 789,099 bytes).

### FIELD OF THE INVENTION

The present invention relates to immunomodulatory molecules that both up-regulate an immune response and down-regulate the immune response, methods of making, and uses thereof.

### BACKGROUND OF THE INVENTION

Current immunotherapy often triggers too much undesired immune response such as immune cell over-activation, cytokine storm, etc.

Cytokines are key regulators of the innate and adaptive immune system that enable immune cells to communicate with each other. Cytokine therapy for activating the immune system of cancer patients continue to be a key area of interest for clinical cancer research. A significant challenge for cytokine monotherapy is to achieve effective anti-tumor responses without causing treatment-limiting toxicities. This dilemma is well exemplified by the low response rates and notorious toxicities of IL-2 and IL-12 therapy. High doses of IL-2 are found to induce vascular leak syndrome (VLS), tumor tolerance caused by activation-induced cell death (AICD), and immunosuppression caused by the activation of regulatory T cells (Tregs). These severe side effects often restrict optimal IL-2 dosing, which limits the number of patients who successfully respond to the therapy. IL-12 has demonstrated modest anti-tumor responses in clinical trials, but often accompanied by significant issues with toxicity (Lasek et al., Cancer Immunol Immunother., 2014). IL-12 treatment was found to associate with systemic flu-like symptoms (e.g., fever, chills, fatigue, erythromelalgia, and headache) and toxic effects on bone marrow and liver. Dosing studies showed that patients could only tolerate IL-12 under 1 μg/kg, far below therapeutically effective dose. Either used as monotherapy or in combination with other agents, IL-12 failed to demonstrate potent sustained therapeutic efficacy in clinical trials (Lasek et al., 2014).

Several approaches have been taken to overcome issues with cytokine monotherapy. Recently, NKTR-214, a recombinant human IL-2 conjugated with polyethylene glycol (PEG; “IL-2-PEG”), has shown promising results in animal models. IL-2-PEG offers two benefits. First, steric hindrance of PEG masks the region on IL-2 that interacts with IL-2 receptor a (IL-2Rα) subunit responsible for activating immunosuppressive Tregs, biasing activity towards tumor killing CD8+ T cells (Charych et al., Clin Cancer Res., 2016). Second, the conjugation of PEG greatly improves plasma half-life and inproteolytic-stability and decreases immunogenicity and hepatic uptake (Chaffee et al., J Clin Invest., 1992; Pyatak et al., Res Commun Chem Pathol Pharmacol., 1980). Targeted delivery of cytokines (e.g., IL-12) to tumor sites by localized injection or by use of immunocytokines (cytokines fused to antibodies, antibody fragments, or ligand/receptor-Fc fusion protein) have also been developed to overcome side effects of cytokine therapy. Immunocytokines can target cytokines to cells or tissues of interest, such as tumor cells or immune effector cells (Klein et al., Oncoimmunology, 2017; King et al., J Clin Oncol., 2004).

The disclosures of all publications, patents, patent applications and published patent applications referred to herein are hereby incorporated herein by reference in their entirety.

### BRIEF SUMMARY OF THE INVENTION

One aspect of the present application provides an immunomodulatory molecule comprising a first binding domain (e.g., immunostimulatory cytokine such as IL-2 or IL-12 or variant thereof) specifically recognizing a first target molecule (e.g., receptor of immunostimulatory cytokine) and a second binding domain (e.g., agonist ligand such as PD-L1 or PD-L2 or variant thereof, or agonist antigen-binding fragment such as anti-PD-1 agonist Fab, scFv, VHH, or full-length antibody) specifically recognizing a second target molecule (e.g., inhibitory checkpoint molecule such as PD-1), wherein the first binding domain upon binding to the first target molecule (e.g., IL-2 or IL-12 receptor) up-regulates an immune response, and wherein the second binding domain upon binding to the second target molecule (e.g., PD-1) down-regulates the immune response

Another aspect of the present application provides a method of modulating an immune response in an individual, comprising administering to the individual an effective amount of any of the immunomodulatory molecules described herein.

Further provided are isolated nucleic acids encoding any one of the immunomodulatory molecules described herein, vectors (e.g., lentiviral vector) comprising such nucleic acids, host cells (e.g., CHO cell) comprising such nucleic acids or vectors, and methods of producing any one of the immunomodulatory molecules described herein.

Also provided are compositions (e.g., pharmaceutical compositions), kits, and articles of manufacture comprising any of the immunomodulatory molecules described herein. Methods of treating a disease or disorder (e.g., cancer, infection, autoimmune disease, allergy, graft rejection, or graft-versus-host disease (GvHD)) in an individual using an effective amount of any of the immunomodulatory molecules or compositions (e.g., pharmaceutical compositions) described herein are also provided.

### BRIEF DESCRIPTION OF THE DRAWINGS

FIGS. 1A-1W depict exemplary immunomodulatory molecule structures of the present invention. FIG. 1A depicts an exemplary immunomodulatory structure comprising a cytokine or variant thereof fused to the N-terminus of a subunit of the Fc fragment of a parental full-length antibody. FIG. 1B depicts a dimeric (homodimeric or heterodimeric) cytokine or variant thereof (e.g., IFN-γ, IL-10, IL-12, or IL-23) expressed in a single chain and positioned at the hinge region of one heavy chain of a parental full-length antibody. FIGS. 1A-1B depict exemplary immunomodulatory structures of the present invention immunostimulatory, in which an immunostimulatory cytokine or variant thereof (e.g., IFN-γ, IL-2, IL-12, or IL-23) expressed in a single chain and positioned at the hinge region of one heavy chain of a dimeric parental ligand/receptor/Fab-hinge-Fc fusion protein. FIG. 1A shows that the Fab of the dimeric parental ligand/receptor/Fab-hinge-Fc fusion protein can be an agonist. FIG. 1B shows that the Fab of the dimeric parental ligand/receptor/Fab-hinge-Fc fusion protein can be an agonist or non-agonist. FIG. 1C depicts an exemplary immunomodulatory structures of the present invention immunostimulatory, in which an immunostimulatory cytokine or variant thereof (e.g., IFN-γ, IL-2, IL-12, or IL-23) expressed in a single chain and positioned at the hinge region of one heavy chain of a parental full-length, agonist antibody (e.g., anti-PD-1 agonist). FIG. 1D depict alternative exemplary immunomodulatory structures of the present invention, in which an immunostimulatory cytokine or variant thereof is positioned between a VH (e.g., within a Fab of an agonist antibody) and a subunit of an Fc fragment. FIG. 1E depicts an exemplary immunomodulatory structure comprising an immunostimulatory cytokine or variant thereof (e.g., IFN-γ, IL-2, IL-12, or IL-23) positioned at the hinge region of one polypeptide of a dimeric parental ligand/receptor-hinge-Fc fusion protein. FIG. 1F depicts an exemplary immunomodulatory structure comprising an immunostimulatory cytokine or variant thereof (e.g., IFN-γ, IL-2, IL-12, or IL-23) fused to the N-terminus of a subunit of the Fc fragment of a parental full-length agonist antibody (e.g., anti-PD agonist). FIG. 1G depicts an exemplary immunomodulatory structure comprising of cytokine or variant thereof (e.g., IFN-γ, IL-2, IL-12, or TL-23) positioned at the hinge region of one polypeptide of a parental ligand/receptor-hinge-Fc fusion protein. FIG. 1H depicts an exemplary immunomodulatory structure comprising of immunostimulatory cytokine or variant thereof (e.g., IFN-γ, IL-2, IL-12, or IL-23) positioned at the hinge region of one polypeptide of a dimeric parental ligand/receptor-hinge-Fc fusion protein. FIG. 1I depicts an exemplary immunomodulatory structures of the present invention immunostimulatory, in which an immunostimulatory cytokine or variant thereof (e.g., IFN-γ, IL-2, IL-12, or IL-23) positioned at the C-terminus of the Fc domain of a parental ligand/receptor-hinge-Fc fusion protein. FIG. 1J depicts an exemplary immunomodulatory structure comprising of an immunostimulatory cytokine or variant thereof (e.g., IFN-γ, IL-2, IL-12, or IL-23) positioned at the C-terminus of the Fc domain of a parental full-length, agonist antibody (e.g., anti-PD-1 agonist). FIG. 1K depicts an exemplary immunomodulatory structure comprising of an immunostimulatory cytokine or variant thereof (e.g., IFN-γ, IL-2, IL-12, or IL-23) positioned at the C-terminus of the Fc domain of a dimeric parental ligand/receptor/agonist Fab-hinge-Fc fusion protein. FIG. 1L depicts two cytokines or variants thereof each positioned at the hinge region of one polypeptide of a parental ligand/receptor-hinge-Fc fusion protein, or a dimeric (homodimeric or heterodimeric) cytokine or variant thereof with each subunit positioned at the hinge region of one polypeptide of a parental ligand/receptor-hinge-Fc fusion protein. FIG. 1M depicts two cytokines or variants thereof each positioned at the hinge region of one polypeptide of a parental ligand/receptor-hinge-Fc fusion protein, or a dimeric (homodimeric or heterodimeric) cytokine or variant thereof with each subunit positioned at the hinge region of one polypeptide of a dimeric parental ligand/receptor-hinge-Fc fusion protein. FIGS. 1N-10 depicts two cytokines or variants thereof each positioned at the hinge region of one polypeptide of a parental full-length, agonist antibody, or a dimeric (homodimeric or heterodimeric) cytokine or variant thereof with each subunit positioned at the hinge region of one polypeptide of a parental full-length, agonist antibody. FIG. 1N shows that the Fab is the same, wherein they are both from an agonist antibody. FIG. 1O shows that the Fab can be different, wherein one is the Fab of an agonist antibody and the other is a different Fab (can be non-agonist or agonist). FIG. 1P depicts two cytokines or variants thereof each positioned at the C-terminus of the Fc domain of a parental ligand/receptor-hinge-Fc fusion protein, or a dimeric (homodimeric or heterodimeric) cytokine or variant thereof with each subunit positioned at the C-terminus of one polypeptide of a parental ligand/receptor-hinge-Fc fusion protein and another at the hinge region of one polypeptide of a parental ligand/receptor-hinge-Fc fusion protein. FIGS. 1Q-1R depicts two cytokines or variants thereof each positioned at the C-terminus of one polypeptide of a parental full-length, antibody, or a dimeric (homodimeric or heterodimeric) cytokine or variant thereof with each subunit positioned at the C-terminus of one polypeptide of Fc domain of agonist antibody and another at the C-terminus of one polypeptide of the Fc domain. FIG. 1Q shows that the Fabs can be the same, wherein they are both from an agonist antibody. FIG. 1R shows that the Fab can be different, wherein one is the Fab of an agonist antibody and the other is a different Fab (can be non-agonist or agonist). FIG. 1S depicts an exemplary immunomodulatory structure comprising an immunostimulatory cytokine or variant thereof fused to the C-terminus of a light chain constant region (CL) of a parental full-length agonist antibody. FIG. 1T depicts an exemplary immunomodulatory structure comprising an immunostimulatory cytokine or variant thereof fused to the N-terminus of a heavy chain variable domain (VH) of a parental full-length antibody. FIG. 1U depicts an exemplary immunomodulatory structure comprising an immunostimulatory cytokine or variant thereof fused to the N-terminus of one polypeptide of a dimeric parental ligand/receptor-hinge-Fc fusion protein. FIG. 1V depicts an exemplary immunomodulatory structure comprising an immunostimulatory cytokine or variant thereof fused to the N-terminus of one polypeptide of a parental ligand/receptor/agonist Fab-hinge-Fc fusion protein. FIG. 1W depicts an exemplary immunomodulatory structure comprising an immunostimulatory cytokine or variant thereof fused to the N-terminus of a heavy chain variable domain (VH) of a parental ligand/receptor/agonist Fab-hinge-Fc fusion protein.

FIGS. 2A-2C depict tumor volume in CT26 syngeneic tumor mice treated with IL-12(F60A)/PD-L2-Fc, hinge (IW-#30) immunomodulatory molecule, IL-12(F60A)/PD-L2-Fc, C-terminus of HC (IW-#34) immunomodulatory molecule, or PBS (negative control). Black arrows indicate injection days. The individual mice responses in each group are given in FIGS. 2B-2C.

FIGS. 3A-3B depict CT26 and EMT6 tumor volume growth overtime in cured CT26 mice (previously cured in FIGS. 2A-2C).

FIG. 4A depicts tumor volume in CT26 syngeneic tumor mice treated with IL-12(E59A/F60A)/PD-L2-Fc, hinge (IW-#29) immunomodulatory molecule, IL-12(F60A)/PD-L2-Fc, hinge (IW-#30) immunomodulatory molecule, or PBS (negative control). Black arrows indicate injection days. FIG. 4B depicts a series of pictures taken of one mouse over the course of treatment with L-12(F60A)/PD-L2-Fc, hinge (IW-#30) immunomodulatory molecule.

FIGS. 5A-5D depict tumor volume in EMT6 syngeneic tumor mice treated with IL-12(E59A/F60A)/PD-L2-Fc, hinge (IW-#29) immunomodulatory molecule, IL-12(F60A)/PD-L2-Fc, hinge (IW-#30) immunomodulatory molecule, IL-12(E59A/F60A)/anti-PD-1, hinge (IW-#48) immunomodulatory molecule, or PBS (negative control). Black arrows indicate injection days. The individual mice responses in each group are given in FIGS. 5B-5D.

FIGS. 6A-6C depict CT26 and EMT6 tumor volume growth overtime in cured EMT6 mice (previously cured in FIGS. 5A-5D).

FIGS. 7A-7D depict tumor volume in 4T1 syngeneic tumor mice treated with increasing concentrations (0, 1, 3, 10, and 50 mg/kg) of IL-12(F60A)/PD-L2-Fc, hinge (IW-#30) immunomodulatory molecule, IL-12(F60A)/PD-L2-Fc, C-terminus of HC (IW-#34) immunomodulatory molecule, IL-12(F60A)/anti-PD-1, hinge (IW-#46) immunomodulatory molecule, IL-12(E59A/F60A)/anti-PD-1, hinge (IW-#48) immunomodulatory molecule, or PBS (negative control). Black arrows indicate injection days.

FIGS. 8A-8C depict tumor volume in B16-F10 syngeneic tumor mice treated with IL-12(F60A)/PD-L2-Fc, hinge (IW-#30) immunomodulatory molecule, PD-L2-Fc/IL-12(F60A) (IW-#34; C-terminal fusion) immunomodulatory molecule, or PBS (negative control). Black arrows indicate injection days. FIG. 8A shows the average tumor volume of all mouse groups, with the average tumor size (±STD) when the first treatment was administered shown in parenthesis. FIGS. 8B-8C show tumor volumes for individual mouse receiving the indicated IL-12 immunomodulatory molecules.

FIGS. 9A-9C depict tumor volume in LL2 syngeneic tumor mice treated with IL-12(F60A)/PD-L2-Fc, hinge (IW-#30) immunomodulatory molecule, IL-12(F60A)/PD-L2-Fc, C-terminus of HC (IW-#34) immunomodulatory molecule, or PBS (negative control). Black arrows indicate injection days. The individual mice responses in each group are given in FIGS. 9B-9C.

FIG. 10 shows two approaches for activating the immune system against a disease (e.g., cancer). The left panel shows a target-independent activation mechanism (“trans-activation”), wherein an immunomodulatory molecule can bind to a target antigen on an immune cell (e.g., a T cell), and can bind to a target antigen on a target cell (e.g., tumor cell), thereby bringing the immune cell into proximity with the target cell for therapeutic effect. This approach, however, can be associated with systemic toxicities, as the binding domain (e.g., wildtype immunostimulatory cytokine, such as IL-12 or IL-2) targeting immune cells can stimulate immune response even in the absence of target cells. The right panel shows target cell-antigen (e.g., tumor antigen) independent activation mechanism (“cis-activation”), in which the immunomodulatory molecules can both up-regulate and down-regulate immune responses, which more closely mimics the natural regulation and balance of the immune system. In a further aspect, immunomodulatory molecules in both left and right panels can further have a “restricted activation mechanism”, in which the first binding domain upon binding to an immune cell upregulating an immune response (e.g., immunostimulatory cytokine, such as IL-12 or IL-2) is modified to reduce activity (binding and/or biological activity), and/or in a “masked” configuration (e.g., positioned at hinge region) until binding of the second binding domain to the second target antigen (e.g., tumor antigen, or immune cell surface molecule) occurs. Exemplary immunomodulatory molecules of the present invention can function via restricted activation, cis-activation, trans-activation, or all mechanisms.

FIGS. 11A-11L depict exemplary multispecific immunomodulatory molecules of the present invention. The immunomodulatory molecules may comprise a variety of combinations of binding domain types. i) a first binding domain, labeled as “1” in the figures, which upon binding to a first target molecule up-regulates an immune response; ii) a second binding domain, labeled as “2” in the figures, which upon binding to a second target molecule down-regulates the immune response; and iii) optionally, a third binding domain, labeled as “3” in the figures, which helps localize the immunomodulatory molecule to a target site (e.g., the tumor microenvironment) by targeting a third target molecule (e.g., marker of exhausted T-cells, T cell surface marker, or tumor antigens). The immunomodulatory molecules can comprise one or more of any of first, second, and/or third binding domain. The multiple first binding domains can be the same or different from each other. The multiple second binding domains can be the same or different from each other. The multiple third binding domains can be the same or different from each other. The various binding domains within the immunomodulatory molecules can be constructed in various configurations, not limited to those shown in FIGS. 11A-11L. As exemplified in FIGS. 11A-11L, the IL-12 moiety (such as a mutant IL-12 moiety with reduced IL-12 activity; either constructed as a single chain fusion, or as two separate subunits) positioned at the C′ of one or both Fc subunits can be a type of first binding domain which upon binding to IL-12R on immune cells up-regulates an immune response. Hence, in FIGS. 11G, 11I, and 11J, the IL-12 moiety functions as “the first binding domain”. The first binding domain (e.g., immunostimulatory cytokine moiety or variant thereof) can be placed at the hinge region between an Fc subunit and the second binding domain or the third binding domain, such as exemplary configurations shown in FIGS. 11A-11F, 11H, 11K, and 11L. Such “restricted access” configurations for first binding domain to its first target molecule can allow i) reduced, minimal or no binding/activity between the first binding domain to its first target molecule in the absence of the binding between the second binding domain to the second target molecule and/or the binding between the third binding domain to the third target molecule (whichever domain that is at N′ of the first binding domain); and ii) rescued/recovered binding/activity of the first binding domain in the presence of the binding between the second binding domain to the second target molecule and/or the binding between the third binding domain to the third target molecule (whichever domain that is at N′ of the first binding domain). The first binding domain (e.g., immunostimulatory cytokine moiety or variant thereof) can also be placed at C′ of one or both Fc subunits of an Fc-fusion protein, such as the IL-12 moiety (either constructed as a single chain fusion and fused to one Fc subunit, or as two separate subunits each fused to one Fc subunit of an Fc domain) exemplified in FIGS. 11A-11L. Such configurations do not or barely restrict binding/activity of the first binding domain.

FIGS. 12A-12D depict exemplary immunomodulatory molecules with a first binding domain (e.g., immunostimulatory cytokines such as IL-12 or variant thereof, for example constructed as a single chain fusion) positioned at the hinge region of one polypeptide chain of a parental (ligand/receptor/antigen binding domain)-hinge-Fc fusion protein, which can be homodimeric or heterodimeric. FIG. 12A depicts an exemplary immunomodulatory molecule, wherein PD-L1 or PD-L2 extracellular domain (wildtype or mutant) is fused to N-terminus of an Fc domain via hinge, and an immunostimulatory cytokine moiety (e.g., IL-12 or variant constructed as a single chain fusion) is positioned at the hinge region of one of the (PD-L1 or PD-L2)-hinge-Fc polypeptide chains. Can be referred to as IL-12/PD-L1-Fc or IL-12/PD-L2-Fc. FIG. 12B depicts an exemplary immunomodulatory molecule wherein PD-L1 or PD-L2 extracellular domain (wildtype or mutant) is fused to the N-terminus of a first Fc subunit via a first hinge, a CD155 extracellular domain (wildtype or mutant) is fused to the N-terminus of a second Fc subunit via a second hinge, and an immunostimulatory cytokine moiety (e.g., IL-12 or variant constructed as a single chain fusion) is positioned at the hinge region of one of the pairing polypeptide chains (such as the PD-L1/PD-L2-hinge-Fc chain). Can be referred to as IL-12/PD-L1-Fc/CD155-Fc or IL-12/PD-L2-Fc/CD155-Fc. FIG. 12C depicts an exemplary immunomodulatory molecule, wherein PD-L1 or PD-L2 extracellular domain (wildtype or mutant) is fused to the N-terminus of a first Fc subunit via a first hinge, an antibody moiety (e.g., sdAb or scFv) specifically recognizing a target molecule (can be agonist, antagonist, or neutral Ab, regulating or not-regulating immune response) is fused to the N-terminus of a second Fc subunit via a second hinge, and an immunostimulatory cytokine moiety (e.g., IL-12 or variant constructed as a single chain fusion) is positioned at the hinge region of one of the pairing polypeptide chains (such as the PD-L1/PD-L2-hinge-Fc chain). Can be referred to as sdAb/IL-12/PD-L1-Fc or sdAb/IL-12/PD-L2-Fc. FIG. 12D depicts an exemplary immunomodulatory molecule, wherein PD-L1 or PD-L2 extracellular domain (wildtype or mutant) is fused to the N-terminus of a first Fc subunit via a first hinge, a Fab specifically recognizing a target molecule (can be agonist, antagonist, or neutral Ab, regulating or not-regulating immune response) is fused through its CH1 to the N-terminus of a second Fc subunit via a second hinge, and an immunostimulatory cytokine moiety (e.g., IL-12 or variant constructed as a single chain fusion) is positioned at the hinge region of one of the pairing polypeptide chains (such as the PD-L1/PD-L2-hinge-Fc chain). Can be referred to as Fab/IL-12/PD-L1-Fc or Fab/IL-12/PD-L2-Fc.

FIGS. 13A-13D depict exemplary immunomodulatory molecules with a first binding domain (e.g., immunostimulatory cytokines such as IL-12 or variant thereof, for example constructed as a single chain fusion) positioned at the C-terminus of the Fc domain (one or both Fc subunits) of a parental (ligand/receptor/antigen binding domain)-hinge-Fc fusion protein, which can be homodimeric or heterodimeric. FIG. 13A depicts an exemplary immunomodulatory molecule wherein PD-L1 or PD-L2 extracellular domain (wildtype or mutant) is fused to N-terminus of an Fc domain via an optional hinge, and an immunostimulatory cytokine moiety (e.g., IL-12 or variant constructed as a single chain fusion) is positioned at the C-terminus one or both subunits of the Fc domain. Can be referred to as PD-L1-Fc/IL-12 or PD-L2-Fc/IL-12. FIG. 13B depicts an exemplary immunomodulatory molecule wherein PD-L1 or PD-L2 extracellular domain (wildtype or mutant) is fused to the N-terminus of a first Fc subunit via a first optional hinge, a CD155 extracellular domain (wildtype or mutant) is fused to the N-terminus of a second Fc subunit via a second optional hinge, and an immunostimulatory cytokine moiety (e.g., IL-12 or variant constructed as a single chain fusion) is positioned at the C-terminus one or both subunits of the Fc domain (such as C′ of the PD-L1/PD-L2-hinge-Fc chain). Can be referred to as PD-L1-Fc/CD155-Fc/IL-12 or PD-L2-Fc/CD155-Fc/IL-12. FIG. 13C depicts an exemplary immunomodulatory molecule, wherein PD-L1 or PD-L2 extracellular domain (wildtype or mutant) is fused to the N-terminus of a first Fc subunit via a first optional hinge, an antibody moiety (e.g., sdAb or scFv) specifically recognizing a target molecule (can be agonist, antagonist, or neutral Ab, regulating or not-regulating immune response) is fused to the N-terminus of a second Fc subunit via a second optional hinge, and an immunostimulatory cytokine moiety (e.g., IL-12 or variant constructed as a single chain fusion) is positioned at the C-terminus one or both subunits of the Fc domain (such as C′ of the PD-L1/PD-L2-hinge-Fc chain). Can be referred to as sdAb/PD-L1-Fc/IL-12 or sdAb/PD-L2-Fc/IL-12. FIG. 13D depicts an exemplary multi-target immunomodulatory molecule wherein PD-L1 or PD-L2 extracellular domain (wildtype or mutant) is fused to the N-terminus of a first Fc subunit via a first optional hinge, a Fab specifically recognizing a target molecule (can be agonist, antagonist, or neutral Ab, regulating or not-regulating immune response) is fused through its CH1 to the N-terminus of a second Fc subunit via a second optional hinge, and an immunostimulatory cytokine moiety (e.g., IL-12 or variant constructed as a single chain fusion) is positioned at the C-terminus one or both subunits of the Fc domain (such as C′ of the PD-L1/PD-L2-hinge-Fc chain). Can be referred to as Fab/PD-L1-Fc/IL-12 or Fab/PD-L2-Fc/IL-12.

FIGS. 14A-14D depict exemplary immunomodulatory molecules with two first binding domains (e.g., immunostimulatory cytokines such as IL-12, IL-2 or variant thereof, for example constructed as a single chain fusion) each positioned at the hinge region of one polypeptide chain of a parental (ligand/receptor/antigen binding domain)-hinge-Fc fusion protein, which can be homodimeric or heterodimeric. FIG. 14A depicts an exemplary immunomodulatory molecule, wherein PD-L1 or PD-L2 extracellular domain (wildtype or mutant) is fused to N-terminus of an Fc domain via hinge, a first immunostimulatory cytokine moiety (e.g., IL-12 or variant constructed as a single chain fusion) is positioned at the hinge region of one of the (PD-L1 or PD-L2)-hinge-Fc polypeptide chains, and a second immunostimulatory cytokine moiety (e.g., IL-2 or variant thereof) is positioned at the hinge region of the other chain of the (PD-L1 or PD-L2)-hinge-Fc polypeptide chains. Can be referred to as IL-12/IL-2/PD-L1-Fc or IL-12/IL-2/PD-L2-Fc. FIG. 14B depicts an exemplary immunomodulatory molecule, wherein PD-L1 or PD-L2 extracellular domain (wildtype or mutant) is fused to the N-terminus of a first Fc subunit via a first hinge, a CD155 extracellular domain (wildtype or mutant) is fused to the N-terminus of a second Fc subunit via a second hinge, a first immunostimulatory cytokine moiety (e.g., IL-12 or variant constructed as a single chain fusion) is positioned at the hinge region of one of the pairing polypeptide chains (such as the PD-L1/PD-L2-hinge-Fc chain), and a second immunostimulatory cytokine moiety (e.g., IL-2 or variant thereof) is positioned at the hinge region of the other chain of the pairing polypeptide chains (such as the CD155-hinge-Fc chain). Can be referred to as IL-12/IL-2/PD-L1-Fc/CD155-Fc or IL-12/IL-2/PD-L2-Fc/CD155-Fc. FIG. 14C depicts an exemplary immunomodulatory molecule, wherein PD-L1 or PD-L2 extracellular domain (wildtype or mutant) is fused to the N-terminus of a first Fc subunit via a first hinge, an antibody moiety (e.g., sdAb or scFv) specifically recognizing a target molecule (can be agonist, antagonist, or neutral Ab, regulating or not-regulating immune response) is fused to the N-terminus of a second Fc subunit via a second hinge, a first immunostimulatory cytokine moiety (e.g., IL-12 or variant constructed as a single chain fusion) is positioned at the hinge region of one of the pairing polypeptide chains (such as the PD-L1/PD-L2-hinge-Fc chain), and a second immunostimulatory cytokine moiety (e.g., IL-2 or variant thereof) is positioned at the hinge region of the other chain of the pairing polypeptide chains (such as the CD155-hinge-Fc chain). Can be referred to as sdAb/IL-12/IL-2/PD-L1-Fc or sdAb/IL-12/IL-2PD-L2-Fc. FIG. 14D depicts an exemplary immunomodulatory molecule, wherein PD-L1 or PD-L2 extracellular domain (wildtype or mutant) is fused to the N-terminus of a first Fc subunit via a first hinge, a Fab specifically recognizing a target molecule (can be agonist, antagonist, or neutral Ab, regulating or not-regulating immune response) is fused through its CH1 to the N-terminus of a second Fc subunit via a second hinge, a first immunostimulatory cytokine moiety (e.g., IL-12 or variant constructed as a single chain fusion) is positioned at the hinge region of one of the pairing polypeptide chains (such as the PD-L1/PD-L2-hinge-Fc chain), and a second immunostimulatory cytokine moiety (e.g., IL-2 or variant thereof) is positioned at the hinge region of the other chain of the pairing polypeptide chains (such as the CD155-hinge-Fc chain). Can be referred to as Fab/IL-12/IL-2/PD-L1-Fc or Fab/IL-12/IL-2/PD-L2-Fc.

FIGS. 15A-15D depict exemplary immunomodulatory molecules with two first binding domains (e.g., immunostimulatory cytokines such as IL-12, IL-2 or variant thereof, for example constructed as a single chain fusion), one is positioned at the hinge region of one polypeptide chain of a parental (ligand/receptor/antigen binding domain)-hinge-Fc fusion protein, and the other one is positioned at the C-terminus of one or both Fc subunits of the parental (ligand/receptor/antigen binding domain)-hinge-Fc fusion protein. FIG. 15A depicts an exemplary immunomodulatory molecule, wherein PD-L1 or PD-L2 extracellular domain (wildtype or mutant) is fused to N-terminus of an Fc domain via hinge, a first immunostimulatory cytokine moiety (e.g., IL-2 or variant) is positioned at the hinge region of one of the (PD-L1 or PD-L2)-hinge-Fc polypeptide chains, and a second immunostimulatory cytokine moiety (e.g., IL-12 or variant constructed as a single chain fusion) is positioned at the C′ of Fc subunit of the other chain of the (PD-L1 or PD-L2)-hinge-Fc polypeptide chains. Can be referred to as TL-2/PD-L1-Fc/IL-12 or IL-2/PD-L2-Fc/IL-12. FIG. 15B depicts an exemplary immunomodulatory molecule, wherein PD-L1 or PD-L2 extracellular domain (wildtype or mutant) is fused to the N-terminus of a first Fc subunit via a first hinge, a CD155 extracellular domain (wildtype or mutant) is fused to the N-terminus of a second Fc subunit via a second hinge, a first immunostimulatory cytokine moiety (e.g., IL-12 or variant constructed as a single chain fusion) is positioned at the C′ of Fc subunit of one of the pairing polypeptide chains (such as the PD-L1/PD-L2-hinge-Fc chain), and a second immunostimulatory cytokine moiety (e.g., IL-2 or variant thereof) is positioned at the hinge region of the other chain of the pairing polypeptide chains (such as the CD155-hinge-Fc chain). Can be referred to as IL-2/PD-L1-Fc/CD155-Fc/IL-12 or LL-2/PD-L2-Fc/CD155-Fc/IL-12. FIG. 15C depicts an exemplary immunomodulatory molecule, wherein PD-L1 or PD-L2 extracellular domain (wildtype or mutant) is fused to the N-terminus of a first Fc subunit via a first hinge, an antibody moiety (e.g., sdAb or scFv) specifically recognizing a target molecule (can be agonist, antagonist, or neutral Ab, regulating or not-regulating immune response) is fused to the N-terminus of a second Fc subunit via a second hinge, a first immunostimulatory cytokine moiety (e.g., IL-12 or variant constructed as a single chain fusion) is positioned at the C′ of Fc subunit of one of the pairing polypeptide chains (such as the PD-L1/PD-L2-hinge-Fc chain), and a second immunostimulatory cytokine moiety (e.g., IL-2 or variant thereof) is positioned at the hinge region of the other chain of the pairing polypeptide chains (such as the CD155-hinge-Fc chain). Can be referred to as sdAb/IL-2/PD-L1-Fc/IL-12 or sdAb/IL-2/PD-L2-Fc/IL-12. FIG. 15D depicts an exemplary immunomodulatory molecule, wherein PD-L1 or PD-L2 (wildtype or mutant) is fused to the N-terminus of a first Fc subunit via a first hinge, a Fab specifically recognizing a target molecule (can be agonist, antagonist, or neutral Ab, regulating or not-regulating immune response) is fused through its CH1 to the N-terminus of a second Fc subunit via a second hinge, a first immunostimulatory cytokine moiety (e.g., IL-12 or variant constructed as a single chain fusion) is positioned at the C′ of Fc subunit of one of the pairing polypeptide chains (such as the PD-L1/PD-L2-hinge-Fc chain), and a second immunostimulatory cytokine moiety (e.g., IL-2 or variant thereof) is positioned at the hinge region of the other chain of the pairing polypeptide chains (such as the CD155-hinge-Fc chain). Can be referred to as Fab/IL-2/PD-L1-Fc/IL-12 or Fab/IL-2/PD-L2-Fc/IL-12.

FIG. 16 shows 4T1 murine breast cancer tumors extracted from mammary gland fat pad of mice treated with IL-12(E59A/F60A)/PD-L2-Fc (IW-#29), IL-12(F60A)/PD-L2-Fc (IW-#30), a combination of anti-PD-1 and anti-CTLA-4 antibodies, or PBS (negative control).

FIG. 17 depicts 4T1 murine breast cancer cells metastasized to lungs in mice injected with 4T1 cells at mammary gland fat pad and treated with IL-12(E59A/F60A)/PD-L2-Fc (IW-#29), IL-12(F60A)/PD-L2-Fc (IW-#30), a combination of anti-PD-1 and anti-CTLA-4 antibodies, or PBS (negative control).

FIG. 18 depicts tumor volume in 4T1 syngeneic tumor mice treated with IL-12(E59A/F60A)/anti-PD-1 (IW-#48), IL-12(E59A/F60A)/PD-L2-Fc (IW-#29), IL-12(E59A/F60A)/IL-2(R38D/K43E/E61R)/anti-PD-1 (IW-#54) immunomodulatory molecules, or PBS (negative control). Black arrows indicate injection days.

FIG. 19 depicts tumor volume in EMT6 syngeneic tumor mice treated with IL-12(E59A/F60A)/anti-PD-1 (IW-#48), IL-12(E59A/F60A)/PD-L2-Fc (IW-#29), IL-2(R38D/K43E/E61R)/PD-L2-Fc (IW-#11) immunomodulatory molecules, or PBS (negative control). Black arrows indicate injection days.

### DETAILED DESCRIPTION OF THE INVENTION

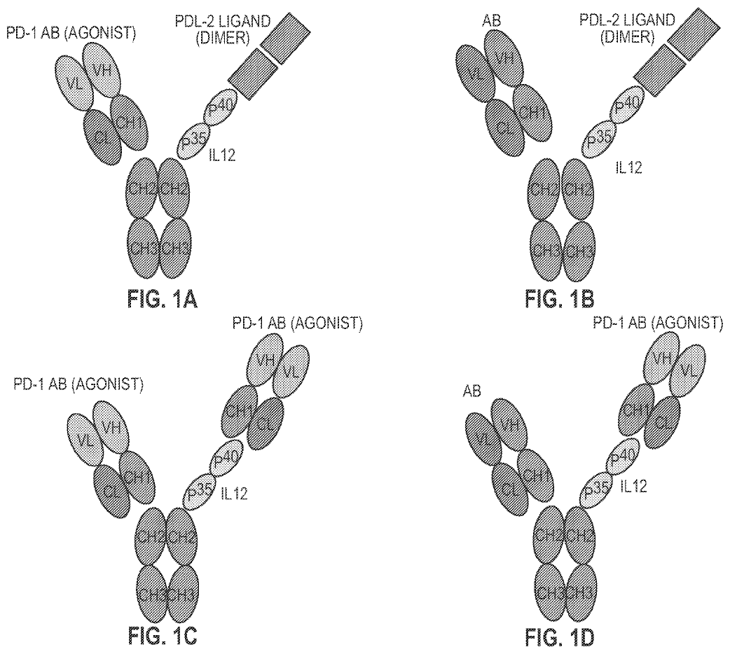

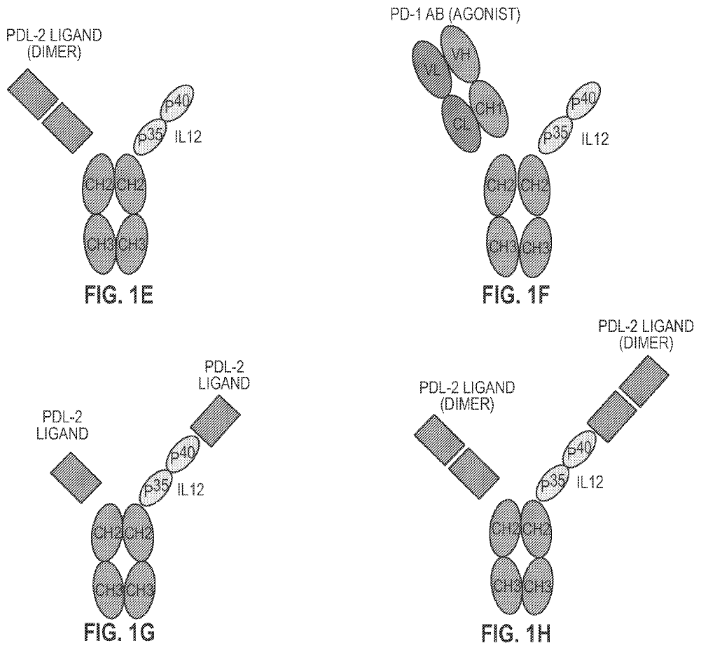

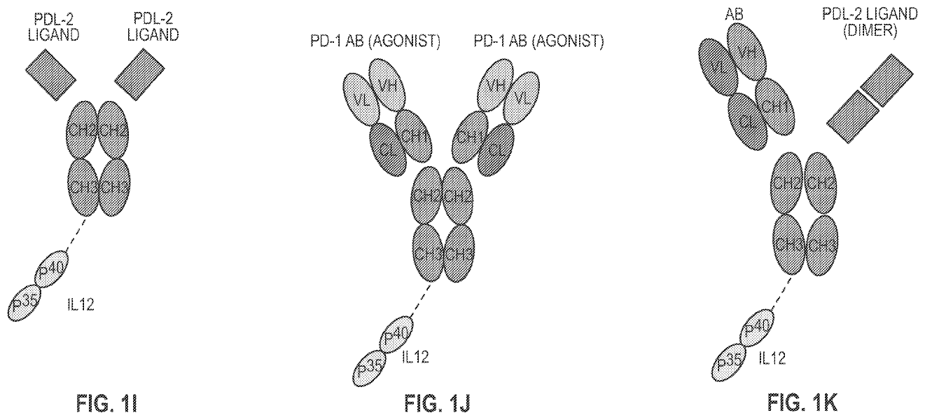

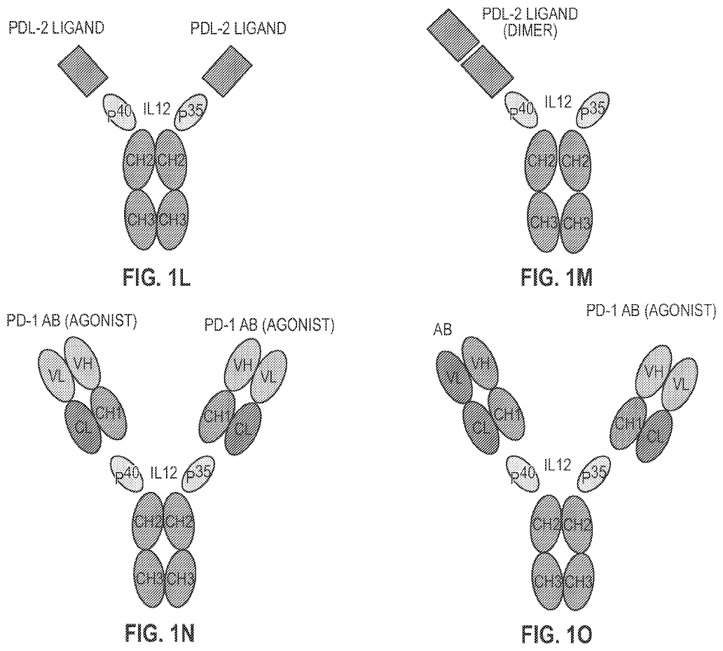

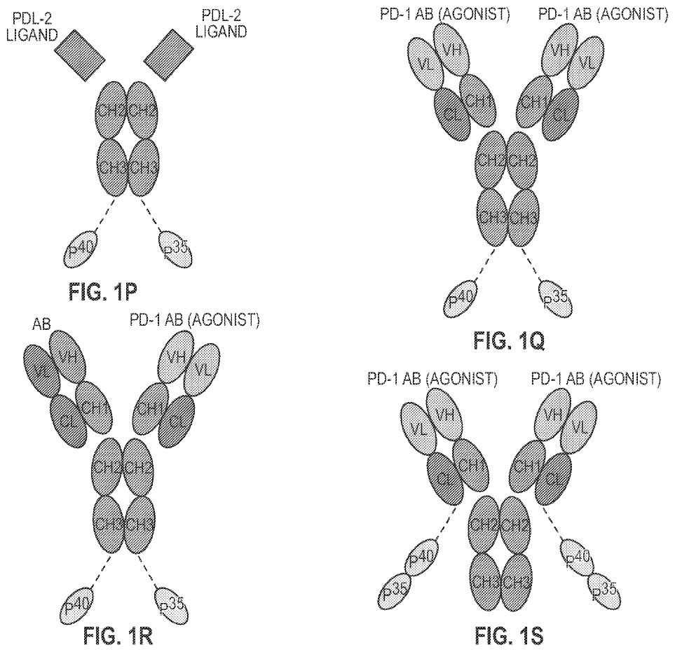

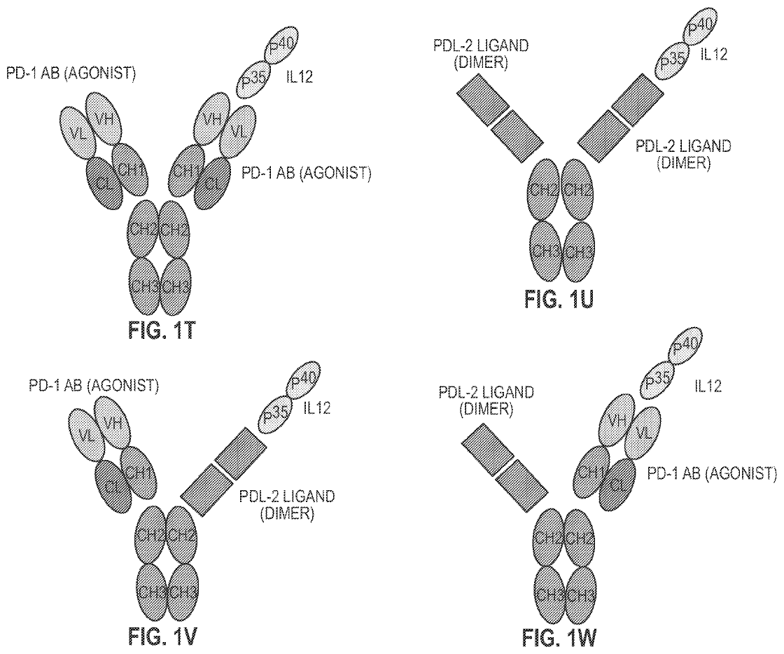

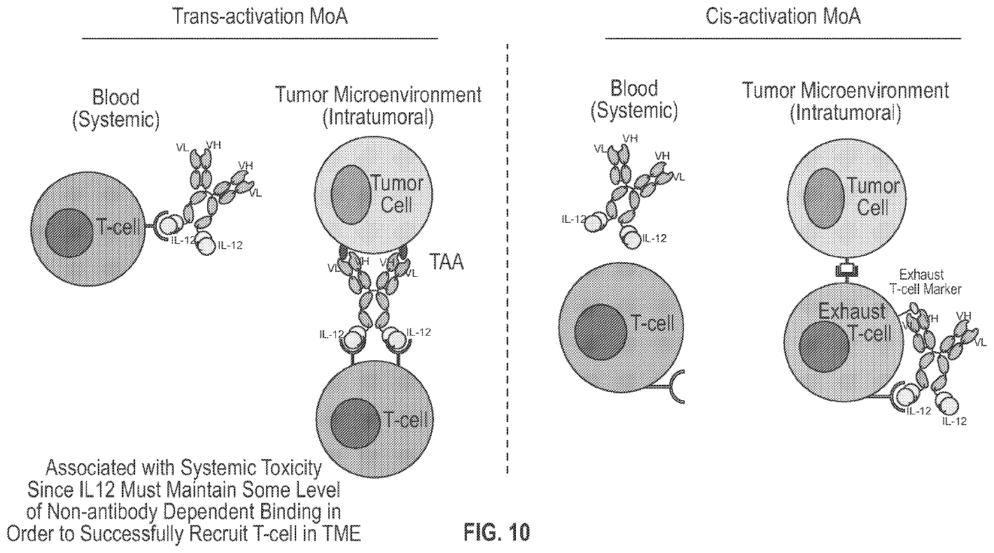

Current immunotherapy often triggers too much undesired immune response such as immune cell over-activation, cytokine storm, etc. For example, cytokine therapy (e.g., for treating cancer) have shown limited success due to severe toxicity, which limits the dosing far below therapeutically effective dose. Immunocytokines, which are constructs with cytokines fused to antibodies, antigen-binding fragments, ligand-Fc fusion protein, or receptor-Fc fusion protein (hereinafter collectively referred to as “ligand/receptor-Fc fusion protein” or “ligand/receptor-hinge-Fc fusion protein”) can deliver cytokines to target cells (e.g., tumor cells, or immune effector cells) or tissues with the recognition of target antigens by the antibodies or antigen-binding fragments (e.g., antibody fragments, ligands, or receptors) within immunomodulatory molecules, which can both reduce non-specific (off-target) cytokine activities and/or associated toxicities (e.g., toxicities on healthy cells or tissues), and concentrate cytokine therapeutic effects at target sites (e.g., disease sites). The activation of immunomodulatory molecules can occur via trans-activation, which requires specific binding of the antibody or antigen-binding fragment to target antigens on tumor cells; or cis-activation, which requires specific binding of the antibody or antigen-binding fragment to target antigens on immune cells (see FIG. 10). Most immunocytokines developed nowadays have the cytokine moiety fused to the N-terminus or the C-terminus of the heavy chain or the light chain of a full-length antibody (such as Hu14.8-IL2, NHS-IL2LT, NHS-IL12, BC1-IL12; see, e.g., FIGS. 1C-IE) or fused to the N-terminus or the C-terminus of an antigen-binding fragment (e.g., diabody, scFv, such as L19-IL2 or F16-IL2), so cytokine-receptor binding/activation can still occur even in the absence of antibody-antigen recognition, leading to off-target toxicities. Immune checkpoint inhibitors developed in recent years (e.g., anti-PD-1, anti-CTLA-4 Abs), although have shown some great clinical success in cancer patients, also focused on up-regulating immune response, which can worsen systemic toxicity if further used together with pro-inflammatory cytokines.

The present invention provides immunomodulatory molecules with opposing effects in regulating immune responses, demonstrated significantly better toxicity profile and therapeutic efficacy. The immunomodulatory molecules comprise a first binding domain (e.g., immunostimulatory cytokine or variant thereof, such as IL-12, IL-2, IFN-γ) specifically recognizing a first target molecule (e.g., receptor of immunostimulatory cytokine or variant thereof) and a second binding domain (e.g., ligand such as PD-L1, PD-L2, CD155 extracellular domain or variant thereof) specifically recognizing a second target molecule (e.g., PD-1 or TIGIT on immune effector cell), wherein the first binding domain upon binding to the first target molecule up-regulates an immune response, and wherein the second binding domain upon binding to the second target molecule down-regulates the immune response. For example, when positioning an IL-12 cytokine (pro-inflammatory) at the hinge region of a PD-L2 extracellular domain-hinge-Fc fusion protein, the resulting IL-12/PD-L2-Fc immunomodulatory molecule not only specifically targeted IL-12 activity (e.g., activity of binding to IL-12 receptor, and/or IL-12 pro-inflammatory activity) to PD-1+target cells, but also stimulated PD-1 inhibitory immune checkpoint signaling via PD-L2-PD-1 binding, thus creating an immunosuppression signal that “balances against” or “counteracts” the immunostimulating activity of IL-12. Any agonist antibodies or ligands (e.g., PD-L2, PD-L1, CD80, or CD86) that can activate or stimulate an immunosuppressive signaling pathway (e.g., by binding to an inhibitory immune checkpoint molecule such as PD-1 or CTLA-4), or any antagonist antibodies, ligands, or receptors that can reduce or block an immunostimulatory signaling pathway (e.g., by binding to a stimulatory immune checkpoint molecule such as CD27 or CD28 or an immunostimulatory receptor such as IL-2R) can be used in combination with an immunostimulating cytokine or variant thereof (e.g., IL-2, IL-12, IFN-γ, or IL-23) to construct an immunomodulatory molecule with any of the immunomodulatory molecule configurations described herein. Any antagonist antibodies, ligands, or receptors that can reduce or block an immunosuppressive signaling pathway (e.g., by binding to an inhibitory immune checkpoint molecule such as PD-1 or CTLA-4), or any agonist antibodies or ligands (e.g., CD70, CD80, CD86, or IL-2) that can activate or stimulate an immunostimulatory signaling pathway (e.g., by binding to a stimulatory immune checkpoint molecule such as CD27 or CD28 or an immunostimulatory receptor such as IL-2R) can be used in combination with an immunosuppressive cytokine or variant thereof (e.g., IL-10, IL-27, IL-35, TGF-β) to construct an immunomodulatory molecule with any of the immunomodulatory molecule configurations described herein. The immunomodulatory molecules described herein can comprise one or more of first binding domains, and/or one or more of second binding domains, in order to achieve multiple immune response regulation. The multiple first binding domains can be the same or different. The multiple second binding domains can be the same or different. See FIGS. 1A-1W and 11A-15D for examples.

The first binding domain can include molecules such as immunostimulatory cytokines, ligands, or agonist antibodies (e.g., ligand or agonist Ab that stimulate stimulatory checkpoint molecules such as OX40), that target immune cells such as T cells, NK cells, DC cells, macrophages, and B cells. The present invention in some embodiments provide first binding domains with reduced activities (e.g., reduced binding or reducing stimulating activity to its target), such as compared to unmodified parental first binding domain. For example, see cytokine variants described herein, which exhibit drastically reduced activity compared to wildtype cytokines. Reducing the binding affinity of the first binding domain can skew the mechanism of action towards target-dependent activation (cis-activation) and away from target-independent activation (trans-activation).

The second binding domain can include molecules such as immunosuppressive cytokines, ligands, or agonist antibodies (e.g., ligand (such as PD-L1, PD-L2, CD155) or agonist Ab that stimulate inhibitory checkpoint molecules such as PD-1 or TIGIT), for down-regulating immune response. The present invention in some embodiments provide anti-PD-1 antibody (antagonist Ab) with reduced binding affinity to PD-1, hence reducing the immune response that could have been induced by a wild-type anti-PD-1 antibody (antagonist Ab, such as nivolumab) (see Example 22). The present invention in some embodiments also provide ligands with increased binding affinity to inhibitory checkpoint molecules such as PD-1, which can further down-regulate immune response compared to wildtype ligands. For example, see mutant PD-L1 and PD-L2 molecules generated in Example 23. Immunomodulatory molecules comprising mutant PD-L1 or PD-L2 extracellular domain as the second binding domain reduced adverse events compared to those with wildtype PD-L1 or PD-L2 extracellular domain. The low-binding affinity of PD-L2(mut) or PD-L1(mut) to PD-1 (more than 10⁻⁸ M K_{d}) compared to wildtype ligand, or the low-binding affinity of the mutant anti-PD-1 antibody (antagonist Ab; more than 10⁻⁸ M K_{d}) compared to wildtype anti-PD-1 antibody (less than 10⁻⁹ M K_{d}), allow immunomodulatory molecules thereof to target cancer cells expressing much higher level of PD-1, such as exhausted T-cells and tumor microenvironments trying the bypass anti-tumor activity, rather than any PD-1 positive cells.

For example, IL-12(E59A/F60A)/PD-L2(S58V)-Fc immunomodulatory molecule described herein provides both positive (IL-12/IL-12R signaling) and negative signals (PD-1/PD-L2 signaling). Immunomodulatory molecules with opposing effects described herein allow mimicking the native T-cell activation process, regulating the T cell activation process, and overcoming over-activation of the immune system.

The immunomodulatory molecules comprising the first and second binding domains described herein can further comprise a third binding domain specifically recognizing a third target molecule. The third binding domain can help localize the immunomodulatory molecule to a target site (e.g., the tumor microenvironment) by binding to the third target molecule (e.g., marker of exhausted T-cells, T cell surface marker, or tumor antigens). The third binding domain upon binding to the third target molecule can i) up-regulate the above mentioned or other immune response, or ii) down-regulate the above mentioned or other immune response; or iii) does not regulate any immune response by its own binding. For example, the third binding domain can function solely as a tumor antigen-targeting domain to bring the immunomodulatory molecule to tumor site, or as an immune effector cell-targeting domain to bring the immunomodulatory molecule to immune effector cells or strengthen its binding to immune effector cells. The intratumoral microenvironment contains a relatively high level of the exhausted T cells expressing several markers, such as TIGIT, TIM3, LAG3, and PD-1. Since the expression pattern and level of exhausted markers in the tumor microenvironment (TME) vary greatly, the third binding domain can be used to target additional exhausted markers to broadly target the TME. Alternatively, the third binding domain can be used to target specific cancers against specific tumor antigen, including but not limited to Her2, CEACAM, Her3, EGFR, Trop2, CLDN18.2, prostate-specific antigen, MUC1, EpCAM, GPC3, mesothelin (MSLN), Nectin4, Folate receptor alpha, tissue factor, etc. The third binding domain may also target T cell markers, including but not limited to CD4, CD8, CD3, CD2, CD5, CD7, CD40L, CD25, CD137, CD69, CTLA4, CD127, ICOS, etc. The third binding domain may also target dendritic cell markers, including but not limited to CD1c, CD11c, CD141, CD123, BDCA-2, BDCA-4, CLEC9A, XCR1, CD80, CD86, PD-L1, PD-L2, etc. The third binding domain may also target monocyte/macrophage markers, including but not limited to CSF1R, CD80, Cd86, CD11, CD14, CD68, CD163, CD16, CD32, CD64, etc. The third binding domain may also target neutrophil cell markers, including but not limited to CD11, CD16, CD32, etc. The immunomodulatory molecules described herein can comprise one or more of third binding domains, in order to achieve multiple immune response regulation or for enhanced targeting. The multiple third binding domains can be the same or different.

Further, the present invention also provides immunomodulatory molecules with certain unique configurations that address the issues faced by current cytokine/immunocytokine therapy. Particularly, some immunomodulatory molecules of the present invention decrease non-specific activities (i.e., antibody or antigen-binding fragment-independent binding) and increase specific activities (i.e., antibody or antigen-binding fragment-dependent binding) of a first binding domain (e.g., immunostimulatory cytokines) by positioning the first binding domain (e.g., cytokine or variant thereof) at a hinge region in between a second binding domain (e.g., ligand, receptor, VHH, scFv, or Fab) and an Fc domain subunit or portion thereof (e.g., CH2-CH3 fragment, or CH2 only, or CH3 only), for example, at a hinge region in between an scFv and an Fc domain subunit (e.g., an antigen-binding polypeptide comprising VH-VL-cytokine-Fc subunit, or VL-VH-cytokine-Fc subunit), at a hinge region in between the Fab and the Fc domain of a full-length antibody (e.g., an antigen-binding polypeptide comprising VH-CH1-cytokine-Fc subunit), or at a hinge region in between a ligand (or a receptor) and an Fc domain subunit (e.g., an antigen-binding polypeptide comprising ligand-cytokine-Fc subunit, or receptor-cytokine-Fc subunit). Without being bound by theory, it is believed that steric hindrance of the second binding domain (e.g., ligand, receptor, VHH, scFv, Fab) and the Fc domain or portion thereof reduces accessibility of the first binding domain (e.g., immunomodulatory cytokine or variant thereof) to its target molecule (e.g., receptor of immunomodulatory cytokine), or “masks” the first binding domain from binding to its first target molecule, in the absence of binding by the second binding domain to the second target molecule. Upon binding of the second binding domain to the second target molecule, on the other hand, the first binding domain becomes activated. Surprisingly, unlike other immunocytokine designs which “expose” the cytokine moiety at its N-terminus or C-terminus, the unique immunomodulatory molecule configuration of the present invention requires binding of the second binding domain (e.g., ligand, receptor, VHH, scFv, or Fab) to its second target molecule first before binding of the first binding domain (e.g., immunomodulatory cytokine moiety) to its first target molecule (e.g., receptor) can occur, thus ensuring that the up-regulation of the immune response (e.g., cytokine signaling activation) is entirely second binding domain-binding dependent (on-target). With this enhanced targeting specificity design, and optionally further in combination with reduced activities of the first binding domain discussed above (e.g., cytokine variants described herein), a desired immune response (e.g., cytokine signaling activation) can be safely delivered to target sites (e.g., tumor cells, or immune cells) to achieve therapeutic effects. Such unique targeting specificity design adds an additional regulatory layer to the current “balancing” or “counteracting” of immune response design, further fine-tuning the bioactivity and toxicity of immunomodulatory molecules described herein.

Accordingly, one aspect of the present application provides an immunomodulatory molecule comprising a first binding domain (e.g., ligand, VHH, scFv, or VH, for example immunostimulatory cytokine such as IL-2 or IL-12) specifically recognizing a first target molecule (e.g., cell surface antigen or receptor, such as receptor of immunostimulatory cytokine) and a second binding domain (e.g., ligand, VHH, scFv, or VH, for example agonist ligand such as PD-L1 or PD-L2, or agonist antigen-binding fragment such as anti-PD-1 agonist Fab, scFv, VH, VHH, or full-length antibody) specifically recognizing a second target molecule (e.g., cell surface antigen or receptor, for example inhibitory checkpoint molecule such as PD-1), wherein the first binding domain upon binding to the first target molecule up-regulates an immune response, and wherein the second binding domain upon binding to the second target molecule down-regulates the immune response.

Also provided are isolated nucleic acids encoding such immunomodulatory molecules, vectors comprising such nucleic acids, host cells comprising such nucleic acids or vectors, methods of producing such immunomodulatory molecules, pharmaceutical compositions and articles of manufacture comprising such immunomodulatory molecules, methods of modulating an immune response with such immunomodulatory molecules or pharmaceutical compositions thereof, and methods of treating diseases (e.g., cancer, viral infection, autoimmune diseases) with such immunomodulatory molecules or pharmaceutical compositions thereof.

### I. Definitions

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., John Wiley & Sons, 1995; Sambrook and Russell, Molecular Cloning: A Laboratory Manual (3rd Edition, 2001); Maniatis et al., Molecular Cloning: A Laboratory Manual (1982); DNA Cloning: A Practical Approach, vol. I&II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R Freshney, ed., 1986); Perbal, A Practical Guide to Molecular Cloning (1984) and other like references.

The term “immunocytokine”, as used herein refers to an antigen-binding protein (e.g., antibody, or antigen-binding fragment (e.g., ligand, receptor, or antibody fragment)) format, which is fused to a cytokine molecule. The antigen-binding protein (e.g., antibody, or antigen-binding fragment (e.g., ligand, receptor, or antibody fragment)) format may be any of those described herein, and the cytokine may be fused directly, or by means of a linker or chemical conjugation to the antigen-binding protein format.

The term “cytokine storm,” also known as a “cytokine cascade” or “hypercytokinemia,” is a potentially fatal immune reaction typically consisting of a positive feedback loop between cytokines and immune cells, with highly elevated levels of various cytokines (e.g., INF-γ, IL-10, IL-6, CCL2, etc.).

As used herein, when a binding domain (e.g., antibody, antigen-binding fragment, or ligand) is referred to as an “antagonist” of a target molecule (e.g., a receptor, or an immune checkpoint molecule), it means that upon target antigen binding, the binding domain (e.g., antibody, antigen-binding fragment, or ligand) blocks, suppresses, or reduces (e.g., reduces at least about any of 10%, 20%, 30%, 40%, 50%, 60%, 70%, 80%, 90%, or 100%) the biological activity of the target molecule (e.g., blocks receptor signaling). For example, an anti-PD-1 antagonist antibody is an antibody that reduces or blocks PD-1 signaling; an antagonist ligand of IL-12 receptor reduces or blocks IL-12 receptor signaling. When a binding domain (e.g., antibody, antigen-binding fragment, or ligand) is referred to as an “agonist” of a target molecule (e.g., a receptor, or an immune checkpoint molecule), it means that upon target molecule binding, the binding domain (e.g., antibody, antigen-binding fragment, or ligand) stimulates, activates, or enhances (e.g., enhances at least about any of 10%, 20%, 30%, 40%, 50%, 60%, 70%, 80%, 90%, 100%, or more) the biological activity of the target molecule (e.g., activates receptor signaling). For example, a wildtype PD-L2 ligand (e.g., extracellular domain) is an agonist that activates PD-1 signaling. For example, an anti-PD-1 agonist antibody is an antibody that induces or enhances PD-1 signaling.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the invention contemplate any one or more of these aspects of treatment. For example, an individual is successfully “treated” if one or more symptoms associated with viral infection are mitigated or eliminated, including, but are not limited to, reducing the proliferation of (or destroying) infectious virus, decreasing symptoms resulting from the disease (e.g., cytokine storm), increasing the quality of life of those suffering from the disease, decreasing the dose of other medications required to treat the disease, and/or prolonging survival of individuals.

The term “prevent,” and similar words such as “prevented,” “preventing” etc., indicate an approach for preventing, inhibiting, or reducing the likelihood of the recurrence of, a disease or condition, e.g., cancer. It also refers to delaying the recurrence of a disease or condition or delaying the recurrence of the symptoms of a disease or condition. As used herein, “prevention” and similar words also includes reducing the intensity, effect, symptoms and/or burden of a disease or condition prior to recurrence of the disease or condition.

As used herein, “delaying” the development of a disease means to defer, hinder, slow, retard, stabilize, and/or postpone development of the disease. This delay can be of varying lengths of time, depending on the history of the disease and/or individual being treated. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to disease (e.g., cancer) progression that may be initially undetectable and includes occurrence, recurrence, and onset.

The term “effective amount” used herein refers to an amount of an agent or a combination of agents, sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) relieve to some extent one or more of the symptoms associated with the cancer; (viii) stimulate or activate immune cells (e.g., immune effector cells), e.g. for immune response, such as to produce cytokine(s), or for immune cell proliferation and/or differentiation; and/or (ix) prevent, reduce, or eliminate inflammation or autoimmune response, such as inhibiting pro-inflammatory cytokine secretion. In the case of viral infection, the effective amount of the agent may inhibit (i.e., reduce to some extent and preferably abolish) virus activity; control and/or attenuate and/or inhibit inflammation or a cytokine storm induced by said viral pathogen; prevent worsening, arrest and/or ameliorate at least one symptom of said viral infection or damage to said subject or an organ or tissue of said subject, emanating from or associated with said viral infection; control, reduce, and/or inhibit cell necrosis in infected and/or non-infected tissue and/or organ; control, ameliorate, and/or prevent the infiltration of inflammatory cells (e.g., NK cells, cytotoxic T cells, neutrophils) in infected or non-infected tissues and/or organs; and/or stimulate or activate immune cells (e.g., immune effector cells), e.g., for immune response, such as to produce cytokine(s), or for immune cell proliferation and/or differentiation.

As used herein, an “individual” or a “subject” refers to a mammal, including, but not limited to, human, bovine, horse, feline, canine, rodent, or primate. In some embodiments, the individual is a human.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody” includes conventional 4-chain antibodies, single-domain antibodies, and antigen-binding fragments thereof.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen-binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four CH domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2.

An “isolated” antibody (or construct) is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator; or (3) to homogeneity by SDS-PAGE under non-reducing or reducing conditions using Coomassie Blue or, preferably, silver stain. Isolated antibody (or construct) includes the antibody in situ within recombinant cells since at least one component of the antibody's natural environment will not be present. Ordinarily, however, an isolated polypeptide, antibody, or construct will be prepared by at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. Heavy-chain only antibodies from the Camelid species have a single heavy chain variable region, which is referred to as “V_{H}H”. V_{H}H is thus a special type of V_{H}.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called complementary determining regions (CDRs) or hypervariable regions (HVRs) both in the heavy chain and light chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975): Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991). Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The terms “full-length antibody”, “intact antibody”, or “whole antibody” are used interchangeably to refer to an antibody in its substantially intact form, as opposed to an antibody fragment. Specifically, full-length 4-chain antibodies include those with heavy and light chains including an Fc region. Full-length heavy-chain only antibodies include the heavy chain variable domain (such as V_{H}H) and an Fc region. The constant domains may be native sequence constant domains (e.g., human native sequence constant domains) or amino acid sequence variants thereof. In some cases, the intact antibody may have one or more effector functions. It is to be understood that for the present invention, reference to a “full-length antibody” also includes a full-length antibody backbone or parental full-length antibody (e.g., full-length 4-chain antibody, or full-length heavy-chain only antibody) whose hinge region has a first binding domain (e.g., cytokine moiety) positioned therein (see, e.g., FIGS. 1C, 1D, 1N, 1O).

An “antibody fragment”, “antigen-binding domain”, or “antigen-binding fragment” comprises a portion of an intact antibody, preferably the antigen binding and/or the variable region of the intact antibody. Examples of antibody fragments include, but are not limited to Fab, Fab′, F(ab′)₂ and Fv fragments; diabodies; linear antibodies (see U.S. Pat. No. 5,641,870, Example 2; Zapata et al., Protein Eng. 8(10): 1057-1062 (1995)); single-chain antibody (scFv) molecules; single-domain antibodies (such as V_{H}H), and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy-terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known. It is to be understood that for the present invention, reference to an “antigen-binding domain” or “antigen-binding fragment” also includes a ligand that can specifically recognizes a target receptor, or a receptor that can specifically recognizes a target ligand.

The term “constant domain” refers to the portion of an immunoglobulin molecule having a more conserved amino acid sequence relative to the other portion of the immunoglobulin, the variable domain, which contains the antigen-binding site. The constant domain contains the CH1, C_{H}2 and C_{H}3 domains (collectively, C_{H}) of the heavy chain and the CHL (or C_{L}) domain of the light chain.

The “heavy chain” of antibodies (immunoglobulins) can be divided into three functional regions: the Fd region, the hinge region, and the Fc region (fragment crystallizable). The Fd region comprises the V_{H} and CH1 domains and, in combination with the light chain, forms Fab—the antigen-binding fragment. The Fc fragment is responsible for the immunoglobulin effector functions, which include, for example, complement fixation and binding to cognate Fc receptors of effector cells. The hinge region, found in IgG, IgA, and IgD immunoglobulin classes, acts as a flexible spacer that allows the Fab portion to move freely in space relative to the Fc region. In contrast to the constant regions, the hinge domains are structurally diverse, varying in both sequence and length among immunoglobulin classes and subclasses. For heavy-chain only antibody, “heavy chain” includes the heavy chain variable domain (such as V_{H}H), a hinge region, and an Fc region. It is to be understood that for the present invention, reference to a “heavy chain” also includes a heavy chain comprising a VH domain, a hinge region, and an Fc domain or portion thereof (e.g., VL-VH-hinge-Fc domain subunit, or VH-VL-hinge-Fc domain subunit), and a heavy chain (e.g., heavy chain of a full-length 4-chain antibody, an VH-hinge-Fc-containing antibody, or heavy chain of a heavy-chain only antibody) comprising a first binding domain (e.g., cytokine moiety) positioned at the hinge region (see, e.g., FIGS. 1C, 1D, 1N, 1O).

The “light chains” of antibodies (immunoglobulins) from any mammalian species can be assigned to one of two clearly distinct types, called kappa (“κ”) and lambda (“λ”), based on the amino acid sequences of their constant domains.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the scFv polypeptide further comprises a polypeptide linker between the V_{H} and V_{L} domains which enables the scFv to form the desired structure for antigen binding. For a review of the scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). “Humanized antibody” is used as a subset of “chimeric antibodies”.

“Humanized” forms of non-human (e.g., llama or camelid) antibodies are chimeric antibodies that contain minimal sequence derived from non-human immunoglobulin. In some embodiments, a humanized antibody is a human immunoglobulin (recipient antibody) in which residues from an CDR (hereinafter defined) of the recipient are replaced by residues from an CDR of a non-human species (donor antibody) such as mouse, rat, rabbit, camel, llama, alpaca, or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995): Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, single-domain antibodies comprise three HVRs (or CDRs): HVR1 (or CDR1), HVR2 (or CDR2), and HVR3 (or CDR3). HVR3 (or CDR3) displays the most diversity of the three HVRs and is believed to play a unique role in conferring fine specificity to antibodies. See, e.g., Hamers-Casterman et al., Nature 363:446-448 (1993); Sheriff et al., Nature Struct. Biol. 3:733-736 (1996).

The term “Complementarity Determining Region” or “CDR” are used to refer to hypervariable regions as defined by the Kabat system. See Kabat et al., Sequences of Proteins of Immunological Interest, 5th Ed. Public Health Service, National Institutes of Health, Bethesda, Md. (1991).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below in Table A.

TABLE A
HVR delineations

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the V_{L} and 26-35 (H1), 50-65 or 49-65 (H2) and 93-102, 94-102, or 95-102 (H3) in the V_{H}. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the V_{L}, the subgroup may be subgroup kappa I, kappa II, kappa III or kappa IV as in Kabat et al., supra. Additionally, for the VH, the subgroup may be subgroup I, subgroup II, or subgroup III as in Kabat et al. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

An “affinity-matured” antibody is one with one or more alterations in one or more CDRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by V_{H}- and V_{L}-domain shuffling. Random mutagenesis of CDR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term “epitope” means a protein determinant capable of specific binding to an antibody or antigen-binding fragment (e.g., ligand, receptor, VHH, scFv, Fab, etc.). Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

As used herein, the term “specifically binds”, “specifically recognizes”, or is “specific for” refers to measurable and reproducible interactions such as binding between a target molecule and a binding domain (or cytokine and cytokine receptor), which is determinative of the presence of the target molecule (or cytokine) in the presence of a heterogeneous population of molecules including biological molecules. For example, an antigen binding protein (such as a Fab) that specifically binds a target molecule (which can be an epitope) is an antigen binding protein that binds this target with greater affinity, avidity, more readily, and/or with greater duration than it binds other target molecules. A cytokine that specifically binds a cytokine receptor is a cytokine that binds this cytokine receptor with greater affinity, avidity, more readily, and/or with greater duration than it binds other cytokine receptors. In some embodiments, the extent of binding of a binding domain (or cytokine) to an unrelated target molecule (or unrelated cytokine receptor) is less than about 10% of the binding of the binding domain (or cytokine) to the target molecule as measured (or the cytokine receptor as measured), e.g., by a radioimmunoassay (RIA). In some embodiments, an antigen binding protein that specifically binds a target (or a cytokine that specifically binds a cytokine receptor) has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antigen binding protein (or cytokine receptor) specifically binds an epitope on a protein (or cytokine) that is conserved among the protein from different species. In some embodiments, specific binding can include, but does not require exclusive binding. Binding specificity of the antigen-binding protein or binding domain (or cytokine and cytokine receptor) can be determined experimentally by any protein binding methods known in the art. Such methods comprise, but are not limited to Western blots, ELISA-, RIA-, ECL-, IRMA-, EIA-, BIACORE™-tests and peptide scans.

The term “specificity” refers to selective recognition of a binding domain for a particular epitope of a target molecule. Natural antibodies, for example, are monospecific. The term “multispecific” as used herein denotes that an antigen binding protein has polyepitopic specificity (i.e., is capable of specifically binding to two, three, or more, different epitopes on one biological molecule or is capable of specifically binding to epitopes on two, three, or more, different biological molecules). “Bispecific” as used herein denotes that an antigen binding protein has two different antigen-binding specificities. Unless otherwise indicated, the order in which the antigens bound by a bispecific antibody listed is arbitrary. That is, for example, the terms “anti-CD3/HER2,” “anti-HER2/CD3,” “CD3×HER2” and “HER2×CD3” may be used interchangeably to refer to bispecific antibodies that specifically bind to both CD3 and HER2. The term “monospecific” as used herein denotes an antigen binding protein that has one or more binding sites each of which bind the same epitope of the same antigen.

The term “valent” as used herein denotes the presence of a specified number of binding sites in an antigen binding protein. A natural antibody for example or a full-length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

“Antibody effector functions” refer to those biological activities attributable to the Fc region (a native sequence Fc region or amino acid sequence variant Fc region) of an antibody and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation. “Reduced or minimized” antibody effector function means that which is reduced by at least 50% (alternatively 60%, 65%, 70%, 75%, 80%, 85%, 90%, 95%, 96%, 97%, 98%, or 99%) from the wild type or unmodified antibody. The determination of antibody effector function is readily determinable and measurable by one of ordinary skill in the art. In a preferred embodiment, the antibody effector functions of complement binding, complement dependent cytotoxicity and antibody dependent cytotoxicity are affected. In some embodiments, effector function is eliminated through a mutation in the constant region that eliminated glycosylation, e.g., “effectorless mutation.” In some embodiments, the effectorless mutation is an N297A or DANA mutation (D265A+N297A) in the C_{H}2 region. Shields et al., J. Biol. Chem. 276 (9): 6591-6604 (2001). Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli.) or in which result in an altered glycosylation pattern that is ineffective or less effective at promoting effector function (e.g., Shinkawa et al., J. Biol. Chem. 278(5): 3466-3473 (2003).

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII, and FcγRIII. Fc expression on hematopoietic cells is summarized in Table 2 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed. Antibody variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

The term “Fc region,” “fragment crystallizable region,” “Fc fragment,” or “Fc domain” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody or Fc-fusion protein, or by recombinantly engineering the nucleic acid encoding a heavy chain of the antibody or Fc-fusion protein. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the immunomodulatory molecules described herein include human IgG1, IgG2 (IgG2A, IgG2B), IgG3 and IgG4.

The term IgG “isotype” or “subclass” as used herein is meant any of the subclasses of immunoglobulins defined by the chemical and antigenic characteristics of their constant regions. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000).

“Fc receptor” or “FcR” describes a receptor that binds the Fc region of an antibody or Fc-fusion protein. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

“Binding affinity” generally refers to the strength of the sum total of non-covalent interactions between a single binding site of a molecule (e.g., an antibody, antigen-binding fragment (such as ligand, receptor, VHH, scFv, etc.), or cytokine) and its binding partner (e.g., an antigen (such as cell surface molecule, receptor, ligand, etc.), or cytokine receptor). Unless indicated otherwise, as used herein, “binding affinity” refers to intrinsic binding affinity that reflects a 1:1 interaction between members of a binding pair. Binding affinity can be indicated by K_{d}, K_{off}, Kₒₙ, or Kₐ. The term “K_{off}”, as used herein, is intended to refer to the off-rate constant for dissociation of an antibody (or antigen-binding fragment) from the antibody (or antigen-binding fragment)/antigen complex (e.g., ligand-receptor complex), or the off rate constant for dissociation of a cytokine from the cytokine/cytokine receptor complex, as determined from a kinetic selection set up, expressed in units of s⁻¹. The term “Kₒₙ”, as used herein, is intended to refer to the on-rate constant for association of an antibody (or antigen-binding fragment) to the antigen to form the antibody (or antigen-binding fragment)/antigen complex, or the on rate constant for association of a cytokine to the cytokine receptor to form the cytokine/cytokine receptor complex, expressed in units of M⁻¹s⁻¹. The term equilibrium dissociation constant “K_{D}” or “K_{d}”, as used herein, refers to the dissociation constant of a particular antibody (or antigen-binding fragment)-antigen interaction (or cytokine-cytokine receptor interaction), and describes the concentration of antigen (or cytokine) required to occupy one half of all of the antibody-binding domains (or antigen-binding fragment) present in a solution of antibody (or antigen-binding fragment) molecules (or cytokine receptor) at equilibrium, and is equal to K_{off}/Kₒₙ, expressed in units of M. The measurement of K_{d} presupposes that all binding agents are in solution. In the case where the antibody (or antigen-binding fragment) is tethered to a cell wall, e.g., in a yeast expression system, the corresponding equilibrium rate constant is expressed as EC50, which gives a good approximation of K_{d}. The affinity constant, Kₐ, is the inverse of the dissociation constant, K_{d}, expressed in units of M⁻¹. The dissociation constant (K_{D} or K_{d}) is used as an indicator showing affinity of antibodies (or antigen-binding fragments) to antigens (or cytokines to cytokine receptors). For example, easy analysis is possible by the Scatchard method using antibodies (or antigen-binding fragments) marked with a variety of marker agents, as well as by using BIACORE™ X (made by Amersham Biosciences), which is an over-the-counter, measuring kit, or similar kit, according to the user's manual and experiment operation method attached with the kit. The K_{D} value that can be derived using these methods is expressed in units of M (Mols). An antibody or antigen-binding fragment thereof (or cytokine) that specifically binds to a target (or cytokine receptor) may have a dissociation constant (K_{d}) of, for example, ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M.

Half maximal inhibitory concentration (IC₅₀) is a measure of the effectiveness of a substance (such as an antibody or antigen-binding fragment) in inhibiting a specific biological or biochemical function. It indicates how much of a particular drug or other substance (inhibitor, such as an antibody or antigen-binding fragment) is needed to inhibit a given biological process by half. The values are typically expressed as molar concentration. IC₅₀ is comparable to an “EC₅₀” for agonist drug or other substance (such as an antibody, antigen-binding fragment, or a cytokine). EC₅₀ also represents the plasma concentration required for obtaining 50% of a maximum effect in vivo. As used herein, an “IC₅₀” is used to indicate the effective concentration of an antibody or antigen-binding fragment needed to neutralize 50% of the antigen bioactivity in vitro. IC₅₀ or EC₅₀ can be measured by bioassays such as inhibition of ligand binding by FACS analysis (competition binding assay), cell-based cytokine release assay, or amplified luminescent proximity homogeneous assay (AlphaLISA).

“Covalent bond” as used herein refers to a stable bond between two atoms sharing one or more electrons. Examples of covalent bonds include, but are not limited to, peptide bonds and disulfide bonds. As used herein, “peptide bond” refers to a covalent bond formed between a carboxyl group of an amino acid and an amine group of an adjacent amino acid. A “disulfide bond” as used herein refers to a covalent bond formed between two sulfur atoms, such as a combination of two Fc fragments (or cytokine subunits) by one or more disulfide bonds. One or more disulfide bonds may be formed between the two fragments by linking the thiol groups in the two fragments. In some embodiments, one or more disulfide bonds can be formed between one or more cysteines of two Fc fragments. Disulfide bonds can be formed by oxidation of two thiol groups. In some embodiments, the covalent linkage is directly linked by a covalent bond. In some embodiments, the covalent linkage is directly linked by a peptide bond or a disulfide bond.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full-length of the sequences being compared.

As used herein, the “C terminus” of a polypeptide refers to the last amino acid residue of the polypeptide which donates its amine group to form a peptide bond with the carboxyl group of its adjacent amino acid residue. “N terminus” of a polypeptide as used herein refers to the first amino acid of the polypeptide which donates its carboxyl group to form a peptide bond with the amine group of its adjacent amino acid residue.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the constructs, polypeptides, and antibodies described herein is in a form other than in the form or setting in which it is found in nature. Isolated nucleic acid molecules therefore are distinguished from nucleic acid encoding the constructs, polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “pharmaceutical formulation” of “pharmaceutical composition” refers to a preparation that is in such form as to permit the biological activity of the active ingredient to be effective, and that contains no additional components that are unacceptably toxic to a subject to which the formulation would be administered. Such formulations are sterile. A “sterile” formulation is aseptic or free from all living microorganisms and their spores.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Immunomodulatory Molecules

The present invention in one aspect provides an immunomodulatory molecule comprising a first binding domain (e.g., immunostimulatory cytokine such as IL-2 or IL-12 or variant thereof) specifically recognizing a first target molecule (e.g., receptor of immunostimulatory cytokine) and a second binding domain (e.g., agonist ligand such as PD-L1 or PD-L2 or variant thereof, or agonist antigen-binding fragment such as anti-PD-1 agonist Fab, scFv, V_{H}H, or full-length antibody) specifically recognizing a second target molecule (e.g., inhibitory checkpoint molecule such as PD-1), wherein the first binding domain upon binding to the first target molecule up-regulates an immune response, and wherein the second binding domain upon binding to the second target molecule down-regulates the immune response. In some embodiments, the immunomodulatory molecule further comprises a third binding domain (e.g., antigen-binding fragment) specifically recognizing a third target molecule, such as a cell surface antigen on an immune effector cell (e.g., CD3, PD-1, CTLA-4) or a cancer cell (e.g., tumor antigen). In some embodiments, the third binding domain upon binding to the third target molecule up-regulate or down-regulate the immune response. In some embodiments, the third binding domain upon binding to the third target molecule does not regulate the immune response.

In some embodiments, the first binding domain and/or the second binding domain and/or the third binding domain is a VHH. In some embodiments, the first binding domain and/or the second binding domain and/or the third binding domain is an scFv. In some embodiments, the first binding domain and/or the second binding domain and/or the third binding domain is a Fab. In some embodiments, the first binding domain and/or the second binding domain and/or the third binding domain is a single chain ligand (e.g., PD-L2 extracellular domain, or cytokine) or receptor. For example, the first domain can be a dimeric cytokine moiety formed by a first cytokine subunit recombinantly linked to a second cytokine subunit via an optional linker. In some embodiments, the first binding domain and/or the second binding domain and/or the third binding domain is a ligand or a receptor formed by two polypeptide chains. For example, the first domain can be a dimeric cytokine moiety formed by a first cytokine subunit in one polypeptide chain and a second cytokine subunit in another polypeptide chain. In some embodiments, the first binding domain or portion thereof is fused to the N-terminus of the second binding domain or portion thereof. In some embodiments, the first binding domain or portion thereof is fused to the C-terminus of the second binding domain or portion thereof. In some embodiments, the first binding domain or portion thereof is fused to the N-terminus of the third binding domain or portion thereof. In some embodiments, the first binding domain or portion thereof is fused to the C-terminus of the third binding domain or portion thereof. In some embodiments, the third binding domain or portion thereof is fused to the N-terminus of the second binding domain or portion thereof. In some embodiments, the third binding domain or portion thereof is fused to the C-terminus of the second binding domain or portion thereof. The immunomodulatory molecules can have any configuration/components exemplified in FIGS. 1A-1W and 11A-15D, and described in any Example and Sequence Listing herein.

In some embodiments, the first binding domain is a VHH. In some embodiments, the first binding domain is an scFv. In some embodiments, the first binding domain is a single chain ligand (e.g., PD-L2, or cytokine) or receptor. In some embodiments, the second binding domain is a Fab. In some embodiments, the first binding domain is fused to the N-terminus of the VH of the Fab. In some embodiments, the first binding domain is fused to the N-terminus of the VL of the Fab. In some embodiments, the first binding domain is fused to the C-terminus of the CH of the Fab. In some embodiments, the first binding domain is fused to the C-terminus of the CL of the Fab. In some embodiments, the first binding domain is a Fab.

In some embodiments, the second binding domain is a VHH. In some embodiments, the second binding domain is an scFv. In some embodiments, the second binding domain is a single chain ligand (e.g., PD-L2, or cytokine) or receptor. In some embodiments, the first binding domain is a Fab. In some embodiments, the second binding domain is fused to the N-terminus of the VH of the Fab. In some embodiments, the second binding domain is fused to the N-terminus of the VL of the Fab. In some embodiments, the second binding domain is fused to the C-terminus of the CH of the Fab. In some embodiments, the second binding domain is fused to the C-terminus of the CL of the Fab. In some embodiments, the second binding domain is a Fab.

In some embodiments, the third binding domain is a VHH. In some embodiments, the third binding domain is an scFv. In some embodiments, the third binding domain is a Fab. In some embodiments, the third binding domain is a ligand or a receptor (e.g., extracellular domain of a ligand or a receptor).

In some embodiments, the first binding domain is positioned at a hinge region of the immunomodulatory molecule, such as at a hinge region between the second binding domain and an Fc domain subunit or portion thereof. In some embodiments, the first binding domain is not positioned at a hinge region of the immunomodulatory molecule, such as is positioned at C′ of one or both Fc subunits of a parental Fc-fusion protein or an Fc-containing parental antibody.

In some embodiments, the immunomodulatory molecule comprises: i) an antigen-binding protein comprising an antigen-binding polypeptide; and ii) the first binding domain (e.g., immunostimulatory cytokine such as IL-2 or IL-12 or variant thereof), wherein the antigen-binding polypeptide comprises from N-terminus to C-terminus: the second binding domain or portion thereof (e.g., agonist ligand such as PD-L1 or PD-L2 or variant thereof, or agonist antigen-binding fragment such as anti-PD-1 agonist Fab, scFv, VHH), a hinge region, and an Fc domain subunit or portion thereof, and wherein the first binding domain is positioned at the hinge region. Thus in some embodiments, there is provided an immunomodulatory molecule comprising i) an antigen-binding protein comprising an antigen-binding polypeptide; and ii) a first binding domain (e.g., immunostimulatory cytokine such as IL-2 or IL-12 or variant thereof) specifically recognizing a first target molecule (e.g., receptor of immunostimulatory cytokine), wherein the antigen-binding polypeptide comprises from N-terminus to C-terminus: a second binding domain or portion thereof (e.g., agonist ligand such as PD-L1 or PD-L2 or variant thereof, or agonist antigen-binding fragment such as anti-PD-1 agonist Fab, scFv, VHH) specifically recognizing a second target molecule (e.g., inhibitory checkpoint molecule such as PD-1), a hinge region, and an Fc domain subunit or portion thereof, wherein the first binding domain is positioned at the hinge region, wherein the first binding domain upon binding to the first target molecule up-regulates an immune response, and wherein the second binding domain upon binding to the second target molecule down-regulates the immune response. In some embodiments, in the presence of binding of the second binding domain to the second target molecule, the activity of the first binding domain increases at least about 20% (such as at least about any of 30%, 40%, 50%, 60%, 70%, 80%, 900%, 100%, 200%, 300%, 400%, 500%, or more) compared to that in the absence of binding of the second binding domain to the second target molecule. In some embodiments, in the absence of binding of the second binding domain to the second target molecule, the activity of the first binding domain positioned at the hinge region is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding first binding domain in a free state. In some embodiments, the antigen-binding protein comprises two antigen-binding polypeptides each comprising a hinge region, and wherein only one antigen-binding polypeptide comprises the first binding domain positioned at the hinge region. In some embodiments, the antigen-binding protein comprises two antigen-binding polypeptides each comprising a hinge region, and wherein each antigen-binding polypeptide comprises a first binding domain positioned at the hinge region. In some embodiments, the immunomodulatory molecule comprises two or more first binding domains, wherein the two or more first binding domains are positioned in tandem at the hinge region of the antigen-binding polypeptide. In some embodiments, the first binding domain is an immunostimulatory cytokine or variant thereof. In some embodiments, the immunostimulatory cytokine is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF. In some embodiments, the first binding domain is an immunostimulatory cytokine variant, and wherein the activity of the immunostimulatory cytokine variant in a free state is no more than about 80% (such as no more than about any of 70%, 60%, 50%, 40%, 30%, 20%, 10%, or 5%) of that of a corresponding wildtype immunostimulatory cytokine in a free state. In some embodiments, the immunostimulatory cytokine or variant thereof is a monomeric immunostimulatory cytokine or variant thereof. In some embodiments, the immunostimulatory cytokine or variant thereof is a dimeric immunostimulatory cytokine or variant thereof. In some embodiments, both subunits of the dimeric immunostimulatory cytokine or variant thereof are positioned in tandem at the hinge region of the antigen-binding polypeptide. In some embodiments, the antigen-binding protein comprises two antigen-binding polypeptides each comprising a hinge region, wherein one subunit of the dimeric immunostimulatory cytokine or variant thereof is positioned at the hinge region of one antigen-binding polypeptide, and wherein the other subunit of the dimeric immunostimulatory cytokine or variant thereof is positioned at the hinge region of the other antigen-binding polypeptide. In some embodiments, the immunostimulatory cytokine or variant thereof is IL-2 or variant thereof. In some embodiments, the TL-2 variant comprises one or more mutations at a position selected from the group consisting of F24, K35, R38, F42, K43, E61, and P65 relative to a wildtype IL-2. In some embodiments, the IL-2 variant comprises one or more mutations selected from the group consisting of F24A, R38D, K43E, E61R, and P65L relative to a wildtype IL-2. In some embodiments, the IL-2 variant comprises an R38D/K43E/E61R mutation relative to a wildtype IL-2. In some embodiments, the immunostimulatory cytokine or variant thereof is IL-12 or variant thereof. In some embodiments, the IL-12 variant comprises one or more mutations within the p40 subunit at a position selected from the group consisting of E45, Q56, V57, K58, E59, F60, G61, D62, A63, G64, Q65, and C177 relative to a wildtype p40 subunit. In some embodiments, the IL-12 variant comprises one or more mutations within the p40 subunit selected from the group consisting of Q56A, V57A, K58A, E59A, F60A, G61A, D62A, A63S, G64A, and Q65A relative to a wildtype p40 subunit. In some embodiments, the IL-12 variant comprises an E59A/F60A mutation within the p40 subunit relative to a wildtype p40 subunit. In some embodiments, the IL-12 variant comprises an F60A mutation within the p40 subunit relative to a wildtype p40 subunit. In some embodiments, the p40 subunit and the p35 subunit of the IL-12 or variant thereof are connected by a linker. In some embodiments, the two or more first binding domains are the same. In some embodiments, the two or more first binding domains are different. In some embodiments, the second binding domain is an agonist ligand or variant thereof of an inhibitory checkpoint molecule. In some embodiments, the inhibitory checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, Siglec7, Siglec9, and VISTA. In some embodiments, the second binding domain is PD-L1 or variant thereof. In some embodiments, the PD-L1 variant has increased binding affinity to PD-1 compared to a wildtype PD-L1. In some embodiments, the PD-L1 variant comprises one or more mutations at a position selected from the group consisting of 154, Y56, E58, R113, M115, S117, and G119 relative to a wildtype PD-L1. In some embodiments, the PD-L1 variant comprises one or more mutations selected from the group consisting of I54Q, Y56F, E58M, R 113T, M115L, S117A, and G119K relative to a wildtype PD-L1. In some embodiments, the PD-L1 variant comprises an I54Q/Y56F/E58M/R113T/M115L/S117A/G119K mutation relative to a wildtype PD-L1. In some embodiments, the second binding domain is PD-L2 or variant thereof. In some embodiments, the PD-L2 variant has increased binding affinity to PD-1 compared to a wildtype PD-L2. In some embodiments, the second binding domain is an agonist antibody or antigen-binding fragment thereof of an inhibitory checkpoint molecule. In some embodiments, the inhibitory checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, Siglec7, Siglec9, and VISTA. In some embodiments, the agonist antibody or antigen-binding fragment thereof specifically recognizes PD-1 (“anti-PD-1 agonist antibody or antigen-binding fragment thereof”). In some embodiments, the agonist antibody or antigen-binding fragment thereof is a Fab. In some embodiments, the agonist antibody or antigen-binding fragment thereof is an scFv. In some embodiments, the antigen-binding protein comprises two or more second binding domains. In some embodiments, two or more second binding domains or portions thereof are positioned in tandem at the N-terminus of the antigen-binding polypeptide. In some embodiments, the antigen-binding protein comprises two antigen-binding polypeptides each comprising a hinge region, and wherein only one antigen-binding polypeptide comprises the two or more second binding domains or portions thereof positioned in tandem at the N-terminus of the antigen-binding polypeptide. In some embodiments, the antigen-binding protein comprises two antigen-binding polypeptides each comprising a hinge region, and wherein each antigen-binding polypeptide comprises one or more second binding domains or portions thereof at the N-terminus of each antigen-binding polypeptide. In some embodiments, the antigen-binding protein comprises two antigen-binding polypeptides each comprising a hinge region, wherein the first antigen-binding polypeptide comprises one or more second binding domains or portions thereof at the N-terminus of the first antigen-binding polypeptide, wherein the second antigen-binding polypeptide comprises a third binding domain or portion thereof at the N-terminus of the second antigen-binding polypeptide, and wherein the third binding domain specifically recognizing a third target molecule. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different.

In some embodiments, there is provided an immunomodulatory molecule comprising: i) a first antigen-binding polypeptide comprising from N-terminus to C-terminus: a first second binding domain (e.g., PD-L2 or PD-L1 or variant thereof), a second second binding domain (e.g., PD-L2 or PD-L1 or variant thereof), a first binding domain (e.g., a p35 subunit and a p40 subunit of an IL-12 or variant thereof (e.g., E59A/F60A or F60A in p40) connected in tandem) positioned at a first hinge region, and a first subunit of an Fc domain or portion thereof; ii) a second antigen-binding polypeptide comprising from N-terminus to C-terminus: a VH, an optional CH1, a second hinge region, and a second subunit of the Fc domain or portion thereof; and iii) a third antigen-binding polypeptide comprising from N-terminus to C-terminus: a VL, and an optional CL; wherein the VH and the VL and optionally the CH1 and the CL form a third binding domain specifically recognizing a third target molecule, wherein the first binding domain specifically recognizes a first target molecule, wherein the second binding domain specifically recognizes a second target molecule (e.g., PD-1), wherein the first binding domain upon binding to the first target molecule (e.g., IL-12 receptor) up-regulates an immune response, and wherein the first and/or second second binding domain (e.g., PD-L2 or PD-L1 or variant thereof) upon binding to the second target molecule down-regulates the immune response. See, e.g., FIG. 1B. In some embodiments, the third binding domain is an agonist antigen-binding fragment specifically recognizing PD-1. See, e.g., FIG. 1A. In some embodiments, the first and second second binding domains are the same. In some embodiments, the first and second second binding domains are different. In some embodiments, the first and second second binding domain specifically recognize the same epitope. In some embodiments, the first and second second binding domain specifically recognize different epitopes.

In some embodiments, there is provided an immunomodulatory molecule comprising: i) a first antigen-binding polypeptide comprising from N-terminus to C-terminus: a first VH, an optional first CH1, a first binding domain (e.g., a p35 subunit and a p40 subunit of an IL-12 or variant thereof connected in tandem) positioned at a first hinge region, and a first subunit of an Fc domain or portion thereof; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form a second binding domain (e.g., an agonist antigen-binding fragment specifically recognizing PD-1) specifically recognizing a second target molecule, wherein the first binding domain specifically recognizes a first target molecule (e.g., IL-12 receptor), wherein the second VH and the second VL and optionally the second CH1 and the second CL form a third binding domain specifically recognizing a third target molecule, wherein the first binding domain upon binding to the first target molecule (e.g., IL-12 receptor) up-regulates an immune response, and wherein the second binding domain upon binding to the second target molecule (e.g., PD-1) down-regulates the immune response. See, e.g., FIG. 1D. In some embodiments, the third binding domain is an agonist antigen-binding fragment specifically recognizing PD-1. Thus in some embodiments, there is provided an immunomodulatory molecule comprising: i) a first antigen-binding polypeptide comprising from N-terminus to C-terminus: a first VH, an optional first CH1, a p35 subunit and a p40 subunit of an IL-12 or variant thereof positioned in tandem at a first hinge region, and a first subunit of an Fc domain or portion thereof; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain which is an agonist antigen-binding fragment specifically recognizing PD-1, wherein the IL-12 or variant upon binding to IL-12 receptor up-regulates an immune response, and wherein the second binding domain upon and/or the third binding domain upon binding to PD-1 down-regulates the immune response. See, e.g., FIG. 1C. In some embodiments, the third binding domain and the second binding domain are the same. In some embodiments, the third binding domain and the second binding domain are different. In some embodiments, the third binding domain and the second binding domain specifically recognize the same epitope. In some embodiments, the third binding domain and the second binding domain specifically recognize different epitopes.

In some embodiments, there is provided an immunomodulatory molecule comprising: i) a first antigen-binding polypeptide comprising from N-terminus to C-terminus: a first second binding domain (e.g., PD-L2 or PD-L1 or variant thereof), a first binding domain (e.g., a p35 subunit and a p40 subunit of an IL-12 or variant thereof connected in tandem) positioned at a first hinge region, and a first subunit of an Fc domain or portion thereof, and ii) a second antigen-binding polypeptide comprising from N-terminus to C-terminus: a second second binding domain (e.g., PD-L2 or PD-L1 or variant thereof), a second hinge region, and a second subunit of an Fc domain or portion thereof, wherein the first binding domain specifically recognizes a first target molecule (e.g., IL-12 receptor), wherein the first binding domain upon binding to the first target molecule (e.g., IL-12 receptor) up-regulates an immune response, and wherein the second binding domain upon binding to the second target molecule (e.g., PD-1) down-regulates the immune response. See, e.g., FIG. 1G. In some embodiments, the first and second second binding domains are the same. In some embodiments, the first and second second binding domains are different. In some embodiments, the first and second second binding domain specifically recognize the same epitope. In some embodiments, the first and second second binding domain specifically recognize different epitopes.

In some embodiments, there is provided an immunomodulatory molecule comprising: i) i) a first antigen-binding polypeptide comprising from N-terminus to C-terminus: a first second binding domain (e.g., PD-L2 or PD-L1 or variant thereof), a second second binding domain (e.g., PD-L2 or PD-L1 or variant thereof), a first binding domain (e.g., a p35 subunit and a p40 subunit of an IL-12 or variant thereof connected in tandem) positioned at a first hinge region, and a first subunit of an Fc domain or portion thereof; and ii) a second antigen-binding polypeptide comprising from N-terminus to C-terminus: a third second binding domain (e.g., PD-L2 or PD-L1 or variant thereof), a fourth second binding domain (e.g., PD-L2 or PD-L1 or variant thereof), a second hinge region, and a second subunit of the Fc domain or portion thereof, wherein the first binding domain specifically recognizes a first target molecule (e.g., IL-12 receptor), wherein the first, second, third, and/or fourth second binding domain specifically recognizes a second target molecule (e.g., PD-1), wherein the first binding domain upon binding to the first target molecule (e.g., IL-12 receptor) up-regulates an immune response, and wherein the first, second, third, and/or fourth second binding domain upon binding to the second target molecule (e.g., PD-1) down-regulates the immune response. See, e.g., FIG. 1H. In some embodiments, the first, second, third, and/or fourth second binding domains are the same. In some embodiments, the first, second, third, and/or fourth second binding domains are different. In some embodiments, the first, second, third, and/or fourth second binding domain specifically recognize the same epitope. In some embodiments, the first, second, third, and/or fourth second binding domain specifically recognize different epitopes.

In some embodiments, there is provided an immunomodulatory molecule comprising: i) a first antigen-binding polypeptide comprising from N-terminus to C-terminus: a first second binding domain (e.g., PD-L2 or PD-L1 or variant thereof), a portion of a first binding domain (e.g., a p35 subunit of an IL-12 or variant thereof) positioned at a first hinge region, and a first subunit of an Fc domain or portion thereof; and ii) a second antigen-binding polypeptide comprising from N-terminus to C-terminus: a second second binding domain (e.g., PD-L2 or PD-L1 or variant thereof), another portion of the first binding domain (e.g., a p40 subunit of an IL-12 or variant thereof) positioned at a second hinge region, and a second subunit of the Fc domain or portion thereof, wherein the first binding domain specifically recognizes a first target molecule (e.g., IL-12 receptor), wherein the first and second second binding domain specifically recognize a second target molecule (e.g., PD-1), wherein the first binding domain upon binding to the first target molecule (e.g., IL-12 receptor) up-regulates an immune response, and wherein the first and/or second second binding domain upon binding to the second target molecule (e.g., PD-1) down-regulates the immune response. See, e.g., FIG. 1L. In some embodiments, the first and second second binding domains are the same. In some embodiments, the first and second second binding domains are different. In some embodiments, the first and second second binding domain specifically recognize the same epitope. In some embodiments, the first and second second binding domain specifically recognize different epitopes.

In some embodiments, there is provided an immunomodulatory molecule comprising: i) a first antigen-binding polypeptide comprising from N-terminus to C-terminus: a portion of a first binding domain (e.g., a p35 subunit or a p40 subunit of an IL-12 or variant thereof) positioned at a first hinge region, and a first subunit of an Fc domain or portion thereof; and ii) a second antigen-binding polypeptide comprising from N-terminus to C-terminus: a first second binding domain (e.g., PD-L2 or PD-L1 or variant thereof), a second second binding domain (e.g., PD-L2 or PD-L1 or variant thereof), another portion of a first binding domain (e.g., a p40 subunit or a p35 subunit of an IL-12 or variant thereof) positioned at a second hinge region, and a second subunit of the Fc domain or portion thereof, wherein the first binding domain specifically recognizes a first target molecule (e.g., IL-12 receptor), wherein the first and second second binding domain specifically recognize a second target molecule (e.g., PD-1), wherein the first binding domain upon binding to the first target molecule (e.g., IL-12 receptor) up-regulates an immune response, and wherein the first and/or second second binding domain upon binding to the second target molecule (e.g., PD-1) down-regulates the immune response. See, e.g., FIG. 1M. In some embodiments, the first and second second binding domains are the same. In some embodiments, the first and second second binding domains are different. In some embodiments, the first and second second binding domain specifically recognize the same epitope. In some embodiments, the first and second second binding domain specifically recognize different epitopes.

In some embodiments, there is provided an immunomodulatory molecule comprising: i) a first antigen-binding polypeptide comprising from N-terminus to C-terminus: a first VH, an optional first CH1, a portion of a first binding domain (e.g., a p35 subunit or a p40 subunit of an IL-12 or variant thereof) positioned at a first hinge region, and a first subunit of an Fc domain or portion thereof; ii) a second antigen-binding polypeptide comprising from N-terminus to C-terminus: a second VH, an optional second CH1, another portion of the first binding domain (e.g., a p40 subunit or a p35 subunit of an IL-12 or variant thereof) positioned at a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form the second binding domain specifically recognizes a second target molecule (e.g., an agonist antigen-binding fragment specifically recognizing PD-1), wherein the second VH and the second VL and optionally the second CH1 and the second CL form a third binding domain specifically recognizing a third target molecule, wherein the first binding domain specifically recognizes a first target molecule (e.g., IL-12 receptor), wherein the first binding domain upon binding to the first target molecule (e.g., IL-12 receptor) up-regulates an immune response, and wherein the second binding domain upon binding to the second target molecule (e.g., PD-1) down-regulates the immune response. See, e.g., FIG. 1O. In some embodiments, the third binding domain is an agonist antigen-binding fragment specifically recognizing PD-1. See, e.g., FIG. 1N. In some embodiments, the third binding domain and the second binding domain are the same. In some embodiments, the third binding domain and the second binding domain are different. In some embodiments, the third binding domain and the second binding domain specifically recognize the same epitope. In some embodiments, the third binding domain and the second binding domain specifically recognize different epitopes.

In some embodiments, the immunomodulatory molecule comprises an antigen-binding protein comprising an antigen-binding polypeptide, wherein the antigen-binding polypeptide comprises from N′ to C′: the first binding domain or portion thereof, the second binding domain or portion thereof, an optional hinge region, and an Fc domain subunit or portion thereof. Thus in some embodiments, there is provided an immunomodulatory molecule comprising an antigen-binding protein comprising an antigen-binding polypeptide, wherein the antigen-binding polypeptide comprises from N′ to C′: the first binding domain or portion thereof (e.g., immunostimulatory cytokine such as IL-2 or IL-12 or variant thereof), the second binding domain or portion thereof (e.g., agonist ligand such as PD-L1 or PD-L2 or variant thereof, or agonist antigen-binding fragment such as anti-PD-1 agonist Fab, scFv, VHH), an optional hinge region, and an Fc domain subunit or portion thereof, wherein the first binding domain specifically recognizes a first target molecule (e.g., IL-12 receptor), wherein the second binding domain specifically recognizes a second target molecule (e.g., PD-1), wherein the first binding domain upon binding to the first target molecule (e.g., IL-12 receptor) up-regulates an immune response, and wherein the second binding domain upon binding to the second target molecule (e.g., PD-1) down-regulates the immune response. In some embodiments, the second binding domain is an agonist Fab or an agonist scFv that specifically recognizes an inhibitory checkpoint molecule. In some embodiments, the second binding domain is an agonist ligand or variant thereof of an inhibitory checkpoint molecule. In some embodiments, the second binding domain is PD-L1 or PD-L2 or variant thereof. In some embodiments, the first binding domain is an immunostimulatory cytokine or variant thereof. In some embodiments, the immunostimulatory cytokine or variant thereof is IL-2 or IL-12 or variant thereof. In some embodiments, wherein the antigen-binding protein comprises two antigen-binding polypeptides each comprising a hinge region, wherein the first antigen-binding polypeptide comprises from N′ to C′: the first binding domain or portion thereof, the second binding domain or portion thereof, a first hinge region, and a first subunit of an Fc domain or portion thereof; wherein the second antigen-binding polypeptide comprises from N′ to C′: a third binding domain or portion thereof, a second hinge region, and a second subunit of the Fc domain or portion thereof; and wherein the third binding domain specifically recognizing a third target molecule. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different.

In some embodiments, there is provided an immunomodulatory molecule comprising: i) a first antigen-binding polypeptide comprising from N-terminus to C-terminus: a first binding domain (e.g., a p35 subunit and a p40 subunit of an IL-12 or variant thereof fused in tandem) specifically recognizing a first target molecule, a first VH, an optional first CH1, a first hinge region, and a first subunit of an Fc domain or portion thereof; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form a second binding domain specifically recognizing a second target molecule (e.g., an agonist antigen-binding fragment specifically recognizing PD-1), wherein the second VH and the second VL and optionally the second CH1 and the second CL form a third binding domain specifically recognizing a third target molecule, wherein the first binding domain upon binding to the first target molecule (e.g., IL-12 receptor) up-regulates an immune response, and wherein the second binding domain upon binding to the second target molecule (e.g., PD-1) down-regulates the immune response. In some embodiments, the third binding domain is an agonist antigen-binding fragment specifically recognizing PD-1. See, e.g., FIG. 1T. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different.

In some embodiments, there is provided an immunomodulatory molecule comprising: i) a first antigen-binding polypeptide comprising from N-terminus to C-terminus: a first binding domain (e.g., a p35 subunit and a p40 subunit of an IL-12 or variant thereof fused in tandem) specifically recognizing a first target molecule, a first second binding domain (e.g., PD-L2 or PD-L1 or variant thereof), a second second binding domain (e.g., PD-L2 or PD-L1 or variant thereof), a first hinge region, and a first subunit of an Fc domain or portion thereof; and ii) a second antigen-binding polypeptide comprising from N-terminus to C-terminus: a third second binding domain (e.g., PD-L2 or PD-L1 or variant thereof), a fourth second binding domain (e.g., PD-L2 or PD-L1 or variant thereof), a second hinge region, and a second subunit of the Fc domain or portion thereof, wherein the first, second, third, and/or fourth second binding domain specifically recognizes a second target molecule (e.g., PD-1), wherein the first binding domain upon binding to the first target molecule (e.g., IL-12 receptor) up-regulates an immune response, and wherein the first, second, third, and/or fourth second binding domain upon binding to the second target molecule (e.g., PD-1) down-regulates the immune response. See, e.g., FIG. 1U. In some embodiments, the first, second, third, and/or fourth second binding domains are the same. In some embodiments, the first, second, third, and/or fourth second binding domains are different. In some embodiments, the first, second, third, and/or fourth second binding domain specifically recognize the same epitope. In some embodiments, the first, second, third, and/or fourth second binding domain specifically recognize different epitopes.

In some embodiments, there is provided an immunomodulatory molecule comprising: i) a first antigen-binding polypeptide comprising from N-terminus to C-terminus: a first binding domain (e.g., a p35 subunit and a p40 subunit of an IL-12 or variant thereof fused in tandem) specifically recognizing a first target molecule, a first second binding domain (e.g., PD-L2 or PD-L1 or variant thereof), a second second binding domain (e.g., PD-L2 or PD-L1 or variant thereof), a first hinge region, and a first subunit of an Fc domain or portion thereof; ii) a second antigen-binding polypeptide comprising from N-terminus to C-terminus: a VH, an optional CH1, a second hinge region, and a second subunit of the Fc domain or portion thereof; and iii) a third antigen-binding polypeptide comprising from N-terminus to C-terminus: a VL, and an optional CL, wherein the VH and the VL and optionally the CH1 and the CL form a third binding domain specifically recognizing a third target molecule, wherein the first and/or second second binding domain specifically recognizes a second target molecule (e.g., PD-1), wherein the first binding domain upon binding to the first target molecule (e.g., IL-12 receptor) up-regulates an immune response, and wherein the first and/or second second binding domain upon binding to the second target molecule (e.g., PD-1) down-regulates the immune response. In some embodiments, the third binding domain is an agonist antigen-binding fragment specifically recognizing PD-1. See, e.g., FIG. 1V. In some embodiments, the first and second second binding domains are the same. In some embodiments, the first and second second binding domains are different. In some embodiments, the first and second second binding domain specifically recognize the same epitope. In some embodiments, the first and second second binding domain specifically recognize different epitopes.

In some embodiments, there is provided an immunomodulatory molecule comprising: i) a first antigen-binding polypeptide comprising from N-terminus to C-terminus: a first binding domain (e.g., a p35 subunit and a p40 subunit of an IL-12 or variant thereof fused in tandem) specifically recognizing a first target molecule, a V_{H}, an optional CH1, a first hinge region, and a first subunit of an Fc domain or portion thereof; ii) a second antigen-binding polypeptide comprising from N-terminus to C-terminus: a first third binding domain (e.g., PD-L2 or PD-L1 or variant thereof), a second third binding domain (e.g., PD-L2 or PD-L1 or variant thereof), a second hinge region, and a second subunit of the Fc domain or portion thereof; and iii) a third antigen-binding polypeptide comprising from N-terminus to C-terminus: a V_{L}, and an optional C_{L}, wherein the V_{H} and the V_{L} and optionally the CH1 and the C_{L} form a second binding domain specifically recognizing a second target molecule (e.g., an agonist antigen-binding fragment specifically recognizing PD-1), wherein the first and/or second third binding domain specifically recognizes a third target molecule (e.g., PD-1), wherein the first binding domain upon binding to the first target molecule (e.g., IL-12 receptor) up-regulates an immune response, and wherein the second binding domain upon binding to the second target molecule (e.g., PD-1) down-regulates the immune response. See, e.g., FIG. 1W. In some embodiments, the first and second third binding domains are the same. In some embodiments, the first and second third binding domains are different. In some embodiments, the first and second third binding domain specifically recognize the same epitope. In some embodiments, the first and second third binding domain specifically recognize different epitopes.

In some embodiments, there is provided an immunomodulatory molecule comprising: i) a first antigen-binding polypeptide comprising from N-terminus to C-terminus: a first VH, an optional first CH1, a first hinge region, and a first subunit of an Fc domain or portion thereof; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a first binding domain (e.g., a p35 subunit and a p40 subunit of an IL-12 or variant thereof fused in tandem) specifically recognizes a first target molecule, a first VL, and an optional first CL; and iv) a fourth antigen-binding polypeptide comprising from N-terminus to C-terminus: a second VL, and an optional second CL, wherein the first VH and the first VL and optionally the first CH1 and the first CL form a second binding domain specifically recognizes a second target molecule (e.g., an agonist antigen-binding fragment specifically recognizing PD-1), and wherein the second VH and the second VL and optionally the second CH1 and the second CL form a third binding domain specifically recognizing a third target molecule, wherein the first binding domain upon binding to the first target molecule (e.g., IL-12 receptor) up-regulates an immune response, and wherein the second binding domain upon binding to the second target molecule (e.g., PD-1) down-regulates the immune response. In some embodiments, the third binding domain is an agonist antigen-binding fragment specifically recognizing PD-1. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different.

In some embodiments, there is provided an immunomodulatory molecule comprising: i) a first antigen-binding polypeptide comprising from N-terminus to C-terminus: a VH, an optional CH1, a first hinge region, and a first subunit of an Fc domain or portion thereof; ii) a second antigen-binding polypeptide comprising from N-terminus to C-terminus: a first third binding domain (e.g., PD-L2 or PD-L1 or variant thereof), a second third binding domain (e.g., PD-L2 or PD-L1 or variant thereof), a second hinge region, and a second subunit of the Fc domain or portion thereof; and iii) a third antigen-binding polypeptide comprising from N-terminus to C-terminus: a first binding domain (e.g., a p35 subunit and a p40 subunit of an IL-12 or variant thereof fused in tandem) specifically recognizing a first target molecule, a VL, and an optional CL, wherein the VH and the VL and optionally the CH1 and the CL form a second binding domain specifically recognizing a second target molecule (e.g., an agonist antigen-binding fragment specifically recognizing PD-1), wherein the first and/or second third binding domain specifically recognizes a third target molecule (e.g., PD-1), wherein the first binding domain upon binding to the first target molecule (e.g., IL-12 receptor) up-regulates an immune response, and wherein the second binding domain upon binding to the second target molecule (e.g., PD-1) down-regulates the immune response. In some embodiments, the first and second third binding domains are the same. In some embodiments, the first and second third binding domains are different. In some embodiments, the first and second third binding domain specifically recognize the same epitope. In some embodiments, the first and second third binding domain specifically recognize different epitopes.

In some embodiments, the immunomodulatory molecule comprises an antigen-binding protein comprising a first antigen-binding polypeptide and a second antigen-binding polypeptide, wherein the first antigen-binding polypeptide comprises from N-terminus to C-terminus: the second antigen binding domain or portion thereof, a first hinge domain, and a first subunit of an Fc domain or portion thereof; wherein the second antigen-binding polypeptide comprises from N-terminus to C-terminus: the first antigen binding domain or portion thereof, a second hinge domain, and a second subunit of the Fc domain or portion thereof. Thus in some embodiments, there is provided an immunomodulatory molecule comprising an antigen-binding protein comprising a first antigen-binding polypeptide and a second antigen-binding polypeptide, wherein the first antigen-binding polypeptide comprises from N-terminus to C-terminus: the second antigen binding domain or portion thereof, a first hinge domain, and a first subunit of an Fc domain or portion thereof; wherein the second antigen-binding polypeptide comprises from N-terminus to C-terminus: the first antigen binding domain or portion thereof, a second hinge domain, and a second subunit of the Fc domain or portion thereof, wherein the first binding domain specifically recognizes a first target molecule, wherein the second binding domain specifically recognizes a second target molecule, wherein the first binding domain upon binding to the first target molecule (e.g., IL-12 receptor) up-regulates an immune response, and wherein the second binding domain upon binding to the second target molecule (e.g., PD-1) down-regulates the immune response. In some embodiments, the second binding domain is an agonist Fab or an agonist scFv that specifically recognizes an inhibitory checkpoint molecule. In some embodiments, the second binding domain is an agonist ligand or variant thereof of an inhibitory checkpoint molecule. In some embodiments, the second binding domain is PD-L1 or PD-L2 or variant thereof. In some embodiments, the first binding domain is an immunostimulatory cytokine or variant thereof. In some embodiments, the immunostimulatory cytokine or variant thereof is IL-2 or IL-12 or variant thereof.

In some embodiments, there is provided an immunomodulatory molecule comprising: i) a first antigen-binding polypeptide comprising from N-terminus to C-terminus: a VH, an optional CH1, a first hinge region, and a first subunit of an Fc domain or portion thereof; ii) a second antigen-binding polypeptide comprising from N-terminus to C-terminus: a first binding domain (e.g., a p35 subunit and a p40 subunit of an IL-12 or variant thereof fused in tandem) specifically recognizes a first target molecule, a second hinge region, and a second subunit of the Fc domain or portion thereof; and iii) a third antigen-binding polypeptide comprising from N-terminus to C-terminus: a VL, and an optional CL, wherein the VH and the VL and optionally the CH1 and the CL form a second binding domain specifically recognizing a second target molecule (e.g., an agonist antigen-binding fragment specifically recognizing PD-1), wherein the first binding domain upon binding to the first target molecule (e.g., IL-12 receptor) up-regulates an immune response, and wherein the second binding domain upon binding to the second target molecule (e.g., PD-1) down-regulates the immune response. See, e.g., FIG. 1F.

In some embodiments, there is provided an immunomodulatory molecule comprising: i) a first antigen-binding polypeptide comprising from N-terminus to C-terminus: a first second binding domain (e.g., PD-L2 or PD-L1 or variant thereof), a second second binding domain (e.g., PD-L2 or PD-L1 or variant thereof), a first hinge region, and a first subunit of an Fc domain or portion thereof; and ii) a second antigen-binding polypeptide comprising from N-terminus to C-terminus: a first binding domain (e.g., a p35 subunit and a p40 subunit of an IL-12 or variant thereof fused in tandem) specifically recognizing a first target molecule, a second hinge region, and a second subunit of the Fc domain or portion thereof, wherein the first and/or second second binding domain specifically recognizes a second target molecule (e.g., PD-1), wherein the first binding domain upon binding to the first target molecule (e.g., IL-12 receptor) up-regulates an immune response, and wherein the first and/or second second binding domain upon binding to the second target molecule (e.g., PD-1) down-regulates the immune response. See, e.g., FIG. 1E. In some embodiments, the first and second second binding domains are the same. In some embodiments, the first and second second binding domains are different. In some embodiments, the first and second second binding domain specifically recognize the same epitope. In some embodiments, the first and second second binding domain specifically recognize different epitopes.

In some embodiments, the immunomodulatory molecule comprises an antigen-binding protein comprising an antigen-binding polypeptide, wherein the antigen-binding polypeptide comprises from N-terminus to C-terminus: the second binding domain or portion thereof, an optional hinge region, an Fc domain subunit or portion thereof, and the first binding domain or portion thereof. Thus in some embodiments, there is provided an immunomodulatory molecule comprising an antigen-binding protein comprising an antigen-binding polypeptide, wherein the antigen-binding polypeptide comprises from N-terminus to C-terminus: a second binding domain or portion thereof, an optional hinge region, an Fc domain subunit or portion thereof, and a first binding domain or portion thereof, wherein the first binding domain specifically recognizes a first target molecule, wherein the second binding domain specifically recognizes a second target molecule (e.g., PD-1), wherein the first binding domain upon binding to the first target molecule (e.g., IL-12 receptor) up-regulates an immune response, and wherein the second binding domain upon binding to the second target molecule (e.g., PD-1) down-regulates the immune response. In some embodiments, the second binding domain is an agonist Fab or an agonist scFv that specifically recognizes an inhibitory checkpoint molecule. In some embodiments, the second binding domain is an agonist ligand or variant thereof of an inhibitory checkpoint molecule. In some embodiments, the second binding domain is PD-L1 or PD-L2 or variant thereof. In some embodiments, the first binding domain is an immunostimulatory cytokine or variant thereof. In some embodiments, the immunostimulatory cytokine or variant thereof is IL-2 or IL-12 or variant thereof. In some embodiments, the immunostimulatory cytokine or variant thereof is a monomeric immunostimulatory cytokine or variant thereof. In some embodiments, the immunostimulatory cytokine or variant thereof is a dimeric immunostimulatory cytokine or variant thereof. In some embodiments, both subunits of the dimeric immunostimulatory cytokine or variant thereof are positioned in tandem at the C-terminus of the antigen-binding polypeptide. In some embodiments, the antigen-binding protein comprises two antigen-binding polypeptides each comprising a hinge region and an Fc domain subunit or portion thereof, wherein one subunit of the dimeric immunostimulatory cytokine or variant thereof is fused to the C-terminus of the Fc domain subunit or portion thereof of one antigen-binding polypeptide, and wherein the other subunit of the dimeric immunostimulatory cytokine or variant thereof is fused to the C-terminus of the Fc domain subunit or portion thereof of the other antigen-binding polypeptide. In some embodiments, wherein the antigen-binding polypeptide not comprising the second binding domain or portion thereof comprises from N-terminus to C-terminus: a third binding domain or portion thereof specifically recognizing a third target molecule, the hinge region, the subunit of the Fc domain or portion thereof, and the subunit of the dimeric immunostimulatory cytokine or variant thereof. In some embodiments, the antigen-binding protein comprises a first antigen-binding polypeptide and a second antigen-binding polypeptide, wherein the first antigen-binding polypeptide comprises from N-terminus to C-terminus: the second binding domain or portion thereof, a first hinge region, a first subunit of an Fc domain or portion thereof, and the first binding domain or portion thereof; wherein the second antigen-binding polypeptide comprises from N′ to C′: a third binding domain or portion thereof specifically recognizing a third target molecule, a second hinge region, and a second subunit of the Fc domain or portion thereof. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different.

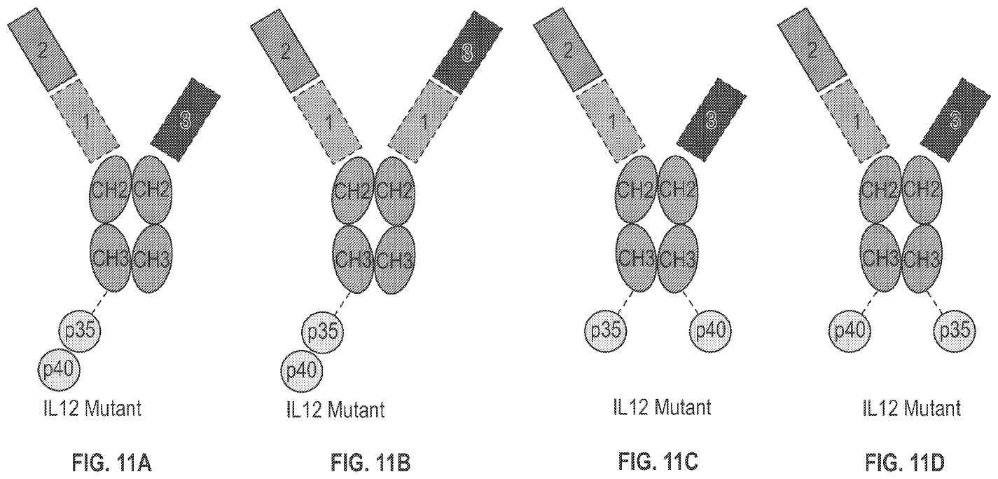

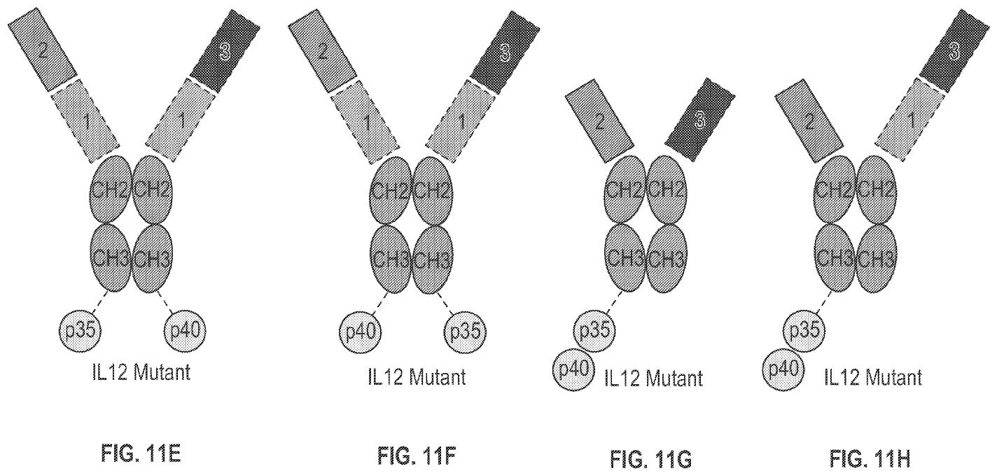

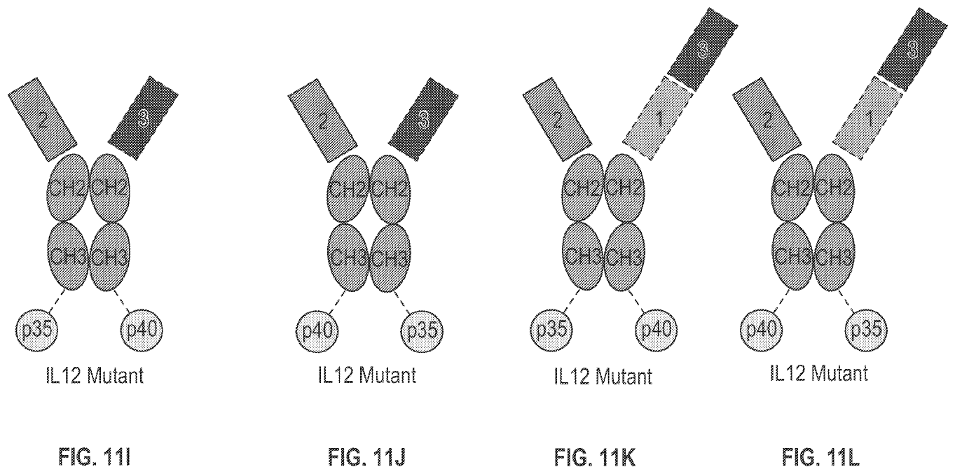

In some embodiments, there is provided an immunomodulatory molecule comprising: i) a first antigen-binding polypeptide comprising from N-terminus to C-terminus: a first second binding domain (e.g., PD-L2 or PD-L1 or variant thereof), a first hinge region, a first subunit of an Fc domain or portion thereof, and a first binding domain (e.g., a p35 subunit and a p40 subunit of an IL-12 or variant thereof fused in tandem) specifically recognizing a first target molecule; and ii) a second antigen-binding polypeptide comprising from N-terminus to C-terminus; a second second binding domain (e.g., PD-L2 or PD-L1 or variant thereof), a second hinge region, and a second subunit of the Fc domain or portion thereof, wherein the first and/or second second binding domain specifically recognizes a second target molecule (e.g., PD-1), wherein the first binding domain upon binding to the first target molecule (e.g., IL-12 receptor) up-regulates an immune response, and wherein the first and/or second second binding domain upon binding to the second target molecule (e.g., PD-1) down-regulates the immune response. See, e.g., FIG. 11.

In some embodiments, there is provided an immunomodulatory molecule comprising: i) a first antigen-binding polypeptide comprising from N-terminus to C-terminus: a first VH, an optional first CH1, a first hinge region, a first subunit of an Fc domain or portion thereof, and a first binding domain (e.g., a p35 subunit and a p40 subunit of an IL-12 or variant thereof fused in tandem) specifically recognizing a first target molecule; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form a second binding domain specifically recognizing a second target molecule (e.g., an agonist antigen-binding fragment specifically recognizing PD-1), wherein the second VH and the second VL and optionally the second CH1 and the second CL form a third binding domain specifically recognizing a third target molecule, wherein the first binding domain upon binding to the first target molecule (e.g., IL-12 receptor) up-regulates an immune response, and wherein the second second binding domain upon binding to the second target molecule (e.g., PD-1) down-regulates the immune response. In some embodiments, the third binding domain is an agonist antigen-binding fragment specifically recognizing PD-1. See, e.g., FIG. 1J. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different.

In some embodiments, there is provided an immunomodulatory molecule comprising: i) a first antigen-binding polypeptide comprising from N-terminus to C-terminus: a VH, an optional CH1, a first hinge region, a first subunit of an Fc domain or portion thereof, and a first binding domain (e.g., a p35 subunit and a p40 subunit of an IL-12 or variant thereof fused in tandem) specifically recognizing a first target molecule; ii) a second antigen-binding polypeptide comprising from N-terminus to C-terminus: a first third binding domain (e.g., PD-L2 or PD-L1 or variant thereof), a second third binding domain (e.g., PD-L2 or PD-L1 or variant thereof), a second hinge region, and a second subunit of the Fc domain or portion thereof; and iii) a third antigen-binding polypeptide comprising from N-terminus to C-terminus: a VL, and an optional CL, wherein the VH and the VL and optionally the CH1 and the CL form a second binding domain specifically recognizing a second target molecule (e.g., an agonist antigen-binding fragment specifically recognizing PD-1), wherein the first binding domain upon binding to the first target molecule (e.g., IL-12 receptor) up-regulates an immune response, and wherein the second second binding domain upon binding to the second target molecule (e.g., PD-1) down-regulates the immune response. See, e.g., FIG. 1K. In some embodiments, the first and second third binding domains are the same. In some embodiments, the first and second third binding domains are different. In some embodiments, the first and second third binding domain specifically recognize the same epitope. In some embodiments, the first and second third binding domain specifically recognize different epitopes.

In some embodiments, there is provided an immunomodulatory molecule comprising: i) a first antigen-binding polypeptide comprising from N-terminus to C-terminus: a first second binding domain (e.g., PD-L2 or PD-L1 or variant thereof), a first hinge region, a first subunit of an Fc domain or portion thereof, and a portion of a first binding domain (e.g., a p35 subunit or a p40 subunit of an IL-12 or variant thereof); and ii) a second antigen-binding polypeptide comprising from N-terminus to C-terminus; a second second binding domain (e.g., PD-L2 or PD-L1 or variant thereof), a second hinge region, and a second subunit of the Fc domain or portion thereof, and another portion of the first binding domain (e.g., a p40 subunit or a p35 subunit of an IL-12 or variant thereof), wherein the first binding domain specifically recognizes a first target molecule (e.g., IL-12 receptor), wherein the first and/or second second binding domain specifically recognizes a second target molecule, wherein the first binding domain upon binding to the first target molecule (e.g., IL-12 receptor) up-regulates an immune response, and wherein the first and/or second second second binding domain upon binding to the second target molecule (e.g., PD-1) down-regulates the immune response. See, e.g., FIG. 1P. In some embodiments, the first and second second binding domains are the same. In some embodiments, the first and second second binding domains are different. In some embodiments, the first and second second binding domain specifically recognize the same epitope. In some embodiments, the first and second second binding domain specifically recognize different epitopes.

In some embodiments, there is provided an immunomodulatory molecule comprising: i) a first antigen-binding polypeptide comprising from N-terminus to C-terminus: a first VH, an optional first CH1, a first hinge region, a first subunit of an Fc domain or portion thereof, and a portion of a first binding domain (e.g., a p35 subunit or a p40 subunit of an IL-12 or variant thereof); ii) a second antigen-binding polypeptide comprising from N-terminus to C-terminus: a second VH, an optional second CH1, a second hinge region, a second subunit of the Fc domain or portion thereof, and another portion of the first binding domain (e.g., a p40 subunit or a p35 subunit of an IL-12 or variant thereof); iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form a second binding domain specifically recognizing a second target molecule (e.g., an agonist antigen-binding fragment specifically recognizing PD-1), and wherein the second VH and the second VL and optionally the second CH1 and the second CL form a third binding domain specifically recognizing a third target molecule, wherein the first binding domain specifically recognizes a first target molecule (e.g., IL-12 receptor), wherein the first binding domain upon binding to the first target molecule (e.g., IL-12 receptor) up-regulates an immune response, and wherein the first and/or second second second binding domain upon binding to the second target molecule (e.g., PD-1) down-regulates the immune response. See, e.g., FIG. 1R. In some embodiments, the third binding domain is an agonist antigen-binding fragment specifically recognizing PD-1. See, e.g., FIG. 1Q. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different.

In some embodiments, the immunomodulatory molecule comprises an antigen-binding protein comprising a first antigen-binding polypeptide and a second antigen-binding polypeptide, wherein the first antigen-binding polypeptide comprises from N-terminus to C-terminus: a VH, a CH1, an optional hinge region, an Fc domain subunit or portion thereof; wherein the second antigen-binding polypeptide comprises from N-terminus to C-terminus: a VL, a CL, and the first binding domain or portion thereof; and wherein the VH and the VL and optionally the CH1 and the CL form the second binding domain. Thus in some embodiments, there is provided an immunomodulatory molecule comprising: i) a first antigen-binding polypeptide and a second antigen-binding polypeptide, wherein the first antigen-binding polypeptide comprises from N-terminus to C-terminus: a VH, a CH1, an optional hinge region, an Fc domain subunit or portion thereof; wherein the second antigen-binding polypeptide comprises from N-terminus to C-terminus: a VL, a CL, and a first binding domain or portion thereof; and wherein the VH and the VL and optionally the CH1 and the CL form a second binding domain specifically recognizes a second target molecule, wherein the first binding domain specifically recognizes a first target molecule (e.g., IL-12 receptor), wherein the first binding domain upon binding to the first target molecule (e.g., IL-12 receptor) up-regulates an immune response, and wherein the second binding domain upon binding to the second target molecule (e.g., PD-1) down-regulates the immune response. In some embodiments, the first antigen-binding polypeptide comprises from N-terminus to C-terminus: a VH, a CH1, a first hinge region, a first subunit of an Fc domain or portion thereof; wherein the antigen-binding protein further comprises a third antigen-binding polypeptide comprising from N-terminus to C-terminus: a third binding domain or portion thereof specifically recognizing a third target molecule, a second hinge region, and a second subunit of the Fc domain or portion thereof. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different. In some embodiments, the immunomodulatory molecule comprises an antigen-binding protein comprising four antigen-binding polypeptides, wherein the first antigen-binding polypeptide comprises from N-terminus to C-terminus: a first VH, a first CH1, a first hinge region, a first subunit of an Fc domain or portion thereof; wherein the second antigen-binding polypeptide comprises from N-terminus to C-terminus: a first VL, a first CL, and the first binding domain or portion thereof, wherein the third antigen-binding polypeptide comprising from N-terminus to C-terminus: a second VH, a second CH1, a second hinge region, and a second subunit of the Fc domain or portion thereof; wherein the fourth antigen-binding polypeptide comprises from N-terminus to C-terminus: a second VL, and a second CL; wherein the first VH and the first VL and the first CH1 and the first CL form the second binding domain; and wherein the second VH and the second VL and the second CH1 and the second CL form a third binding domain specifically recognizing a third target molecule. In some embodiments, the first binding domain is an immunostimulatory cytokine or variant thereof. In some embodiments, the immunostimulatory cytokine or variant thereof is IL-2 or IL-12 or variant thereof. In some embodiments, the immunostimulatory cytokine or variant thereof is a monomeric immunostimulatory cytokine or variant thereof. In some embodiments, the immunostimulatory cytokine or variant thereof is a dimeric immunostimulatory cytokine or variant thereof. In some embodiments, the dimeric immunostimulatory cytokine or variant thereof are positioned in tandem at the C-terminus of the second antigen-binding polypeptide and/or the fourth antigen-binding polypeptide. In some embodiments, one subunit of the dimeric immunostimulatory cytokine or variant thereof is fused to the C-terminus of the first CL of the second antigen-binding polypeptide, and wherein the other subunit of the dimeric immunostimulatory cytokine or variant thereof is fused to the second CL of the fourth antigen-binding polypeptide.

In some embodiments, there is provided an immunomodulatory molecule comprising: i) a first antigen-binding polypeptide comprising from N-terminus to C-terminus: a first VH, a first CH1, a first hinge region, and a first subunit of an Fc domain or portion thereof; ii) a second antigen-binding polypeptide comprising from N-terminus to C-terminus: a first VL, a first CL, and a first first binding domain (e.g., a p35 subunit and a p40 subunit of an IL-12 or variant thereof fused in tandem); iii) a third antigen-binding polypeptide comprising from N-terminus to C-terminus: a second VH, a second CH1, a second hinge region, and a second subunit of the Fc domain or portion thereof; and iv) a fourth antigen-binding polypeptide comprising from N-terminus to C-terminus: a second VL, a second CL, and a second first binding domain (e.g., a p35 subunit and a p40 subunit of an IL-12 or variant thereof fused in tandem); wherein the first VH and the first VL and the first CH1 and the first CL form a second binding domain specifically recognizing a second target molecule (e.g., an agonist antigen-binding fragment specifically recognizing PD-1), and wherein the second VH and the second VL and the second CH1 and the second CL form a third binding domain specifically recognizing a third target molecule, wherein the first and/or second first binding domain specifically recognizes a first target molecule (e.g., IL-12 receptor), wherein the first and/or second first binding domain upon binding to the first target molecule (e.g., IL-12 receptor) up-regulates an immune response, and wherein the second binding domain upon binding to the second target molecule (e.g., PD-1) down-regulates the immune response. In some embodiments, the third binding domain is an agonist antigen-binding fragment specifically recognizing PD-1. See, e.g., FIG. 1S. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different. In some embodiments, the first and second first binding domains are the same. In some embodiments, the first and second first binding domains are different. In some embodiments, the first and second first binding domains specifically recognize the same epitope. In some embodiments, the first and second first binding domains specifically recognize different epitopes.

In some embodiments, there is provided an immunomodulatory molecule comprising: i) a first antigen-binding polypeptide comprising from N-terminus to C-terminus: a second binding domain, a first first binding domain, a first hinge region, a first subunit of an Fc domain or portion thereof, and a second first binding domain (e.g., a p35 subunit and a p40 subunit of an IL-12 or variant thereof connected in tandem; and ii) a second antigen-binding polypeptide comprising from N-terminus to C-terminus: a third binding domain, optionally a third first binding domain, a second hinge region, and a second subunit of the Fc domain or portion thereof, wherein the first first binding domain specifically recognizes a first target molecule, wherein the second first binding domain specifically recognizes a second target molecule (e.g., IL-12 receptor), wherein the second binding domain specifically recognizes a third target molecule, wherein the third binding domain specifically recognizes a fourth target molecule, optionally wherein the optional third first binding domain recognizes a fifth target molecule, wherein the first first binding domain upon binding to the first target molecule up-regulates an immune response, wherein the second first binding domain upon binding to the second target molecule up-regulates an immune response, wherein the second binding domain upon binding to the third target molecule down-regulates the immune response, wherein the third binding domain upon binding to the fourth target molecule localize the immunomodulatory molecule to a tumor microenvironment, and optionally wherein the third first binding domain upon binding to the fifth target molecule up-regulates an immune response. See, e.g., FIG. 11A-11B. In some embodiments, the first, second, and/or third first binding domains are different. In some embodiments, the first, second, and/or third first binding domain specifically recognize the same epitope. In some embodiments, the first, second, and/or third first binding domain specifically recognize different epitopes.

In some embodiments, there is provided an immunomodulatory molecule comprising: i) a first antigen-binding polypeptide comprising from N-terminus to C-terminus: a second binding domain, a first first binding domain, a first hinge region, a first subunit of an Fc domain or portion thereof, and a second first binding domain subunit (e.g., a p35 subunit or a p40 subunit of an IL-12 or variant thereof); and ii) a second antigen-binding polypeptide comprising from N-terminus to C-terminus: a third binding domain, optionally a third first binding domain, a second hinge region, a second subunit of the Fc domain or portion thereof, and a second first binding domain subunit (e.g., a p35 subunit or a p40 subunit of an IL-12 or variant thereof), wherein the first first binding domain specifically recognizes a first target molecule, wherein the second first binding domain specifically recognizes a second target molecule (e.g., IL-12 receptor), wherein the second binding domain specifically recognizes a third target molecule, wherein the third binding domain specifically recognizes a fourth target molecule, optionally wherein the optional third first binding domain recognizes a fifth target molecule, wherein the first first binding domain upon binding to the first target molecule up-regulates an immune response, wherein the second first binding domain upon binding to the second target molecule up-regulates an immune response, wherein the second binding domain upon binding to the third target molecule down-regulates the immune response, wherein the third binding domain upon binding to the fourth target molecule localize the immunomodulatory molecule to a tumor microenvironment, and optionally wherein the third first binding domain upon binding to the fifth target molecule up-regulates an immune response. See, e.g., FIG. 11C-11F. In some embodiments, the first, second, and/or third first binding domains are different. In some embodiments, the first, second, and/or third first binding domain specifically recognize the same epitope. In some embodiments, the first, second, and/or third first binding domain specifically recognize different epitopes.

In some embodiments, there is provided an immunomodulatory molecule comprising: i) a first antigen-binding polypeptide comprising from N-terminus to C-terminus: a second binding domain, a first hinge region, a first subunit of an Fc domain or portion thereof, and a first first binding domain subunit (e.g., a p35 subunit or a p40 subunit of an IL-12 or variant thereof); and ii) a second antigen-binding polypeptide comprising from N-terminus to C-terminus: a third binding domain, optionally a second first binding domain, a second hinge region, a second subunit of the Fc domain or portion thereof, and a first first binding domain subunit (e.g., a p35 subunit or a p40 subunit of an IL-12 or variant thereof), wherein the first first binding domain specifically recognizes a first target molecule (e.g., IL-12 receptor), wherein the second binding domain specifically recognizes a second target molecule, wherein the third binding domain specifically recognizes a third target molecule, optionally wherein the optional second first binding domain recognizes a fourth target molecule, wherein the first first binding domain upon binding to the first target molecule up-regulates an immune response, wherein the second binding domain upon binding to the second target molecule down-regulates the immune response, wherein the third binding domain upon binding to the third target molecule localize the immunomodulatory molecule to a tumor microenvironment, and optionally wherein the second first binding domain upon binding to the fourth target molecule up-regulates an immune response. See, e.g., FIG. 11I-11L. In some embodiments, the first and/or second first binding domains are different. In some embodiments, the first and/or second first binding domain specifically recognize the same epitope. In some embodiments, the first and/or second first binding domain specifically recognize different epitopes.

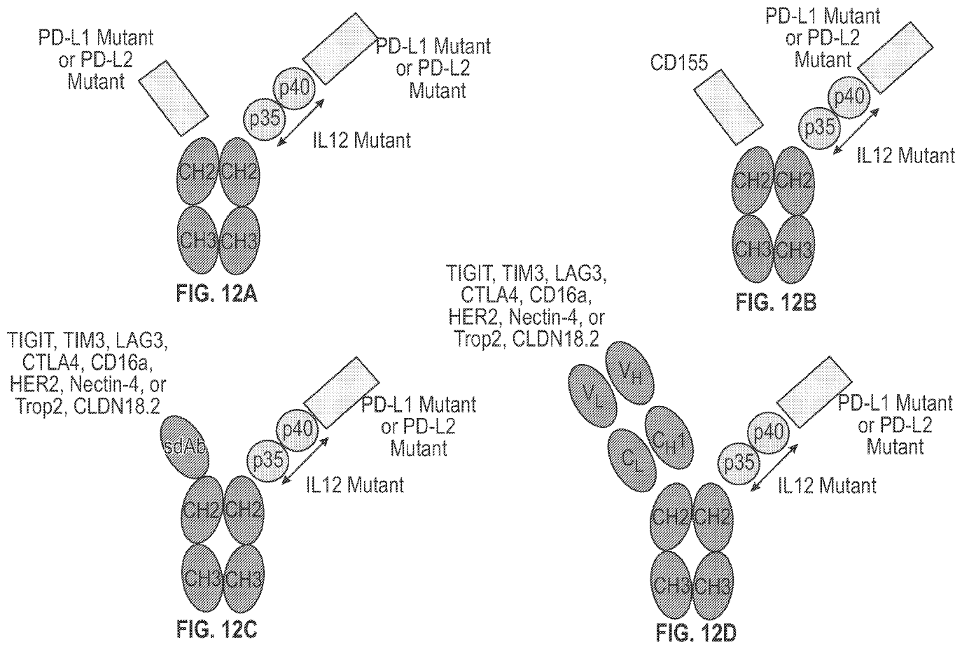

In some embodiments, there is provided an immunomodulatory molecule comprising: i) a first antigen-binding polypeptide comprising from N-terminus to C-terminus: a first second binding domain (e.g., PD-L2 or PD-L1 or variant thereof) positioned at the first hinge region, and a first subunit of an Fc domain or portion thereof, and ii) a second antigen-binding polypeptide comprising from N-terminus to C-terminus: a second second binding domain (e.g., PD-L2 or PD-L1 or variant thereof), a first binding domain (e.g., a p35 subunit and a p40 subunit of an IL-12 or variant thereof connected in tandem), a second hinge region, and a second subunit of the Fc domain or portion thereof, wherein the first binding domain specifically recognizes a first target molecule (e.g., IL-12 receptor), wherein the first and/or second second binding domain specifically recognizes a second target molecule (e.g., PD-1), wherein the first binding domain upon binding to the first target molecule (e.g., IL-12 receptor) up-regulates an immune response, and wherein the first and/or second second binding domain upon binding to the second target molecule (e.g., PD-1) down-regulates the immune response. See, e.g., FIG. 12A. In some embodiments, the first and/or second second binding domains are the same. In some embodiments, the first and/or second second binding domains are different. In some embodiments, the first and/or second second binding domain specifically recognize the same epitope. In some embodiments, the first and/or second second binding domain specifically recognize different epitopes.

In some embodiments, there is provided an immunomodulatory molecule comprising: i) a first antigen-binding polypeptide comprising from N-terminus to C-terminus: a first second binding domain (e.g., CD155 or variant thereof) positioned at the first hinge region, and a first subunit of an Fc domain or portion thereof; and ii) a second antigen-binding polypeptide comprising from N-terminus to C-terminus: a second second binding domain (e.g., PD-L2 or PD-L1 or variant thereof), a first binding domain (e.g., a p35 subunit and a p40 subunit of an IL-12 or variant thereof connected in tandem), a second hinge region, and a second subunit of the Fc domain or portion thereof, wherein the first binding domain specifically recognizes a first target molecule (e.g., IL-12 receptor), wherein the first second binding domain specifically recognizes a second target molecule (e.g., TIGIT), wherein the second second binding domain specifically recognizes a third target molecule (e.g. PD-1), wherein the first binding domain upon binding to the first target molecule (e.g., IL-12 receptor) up-regulates an immune response, and wherein the first and/or second second binding domain upon binding to the second target molecule (e.g., TIGIT and/or PD-1) down-regulates the immune response. See, e.g., FIG. 12B.

In some embodiments, there is provided an immunomodulatory molecule comprising: i) a first antigen-binding polypeptide comprising from N-terminus to C-terminus: a third binding domain (e.g. sdAb), a first hinge region, and a first subunit of an Fc domain or portion thereof, and ii) a second antigen-binding polypeptide comprising from N-terminus to C-terminus: a second binding domain (e.g., PD-L2 or PD-L1 or variant thereof), a first binding domain (e.g., a p35 subunit and a p40 subunit of an IL-12 or variant thereof connected in tandem), a second hinge region, and a second subunit of the Fc domain or portion thereof, wherein the first binding domain upon binding to the first target molecule (e.g., IL-12 receptor) up-regulates an immune response, wherein the second binding domain specifically recognizes a second target molecule (e.g., PD-1), wherein the third binding domain specifically recognizes a third target molecule (e.g. TIGIT, TIM3, LAG3, CTLA4, CD16A, HER2, Nectin-4, Trop2, or CLDN18.2, or variants thereof), wherein the first binding domain upon binding to the first target molecule (e.g., IL-12 receptor) up-regulates an immune response, and wherein the second binding domain upon binding to the second target molecule (e.g., PD-1) down-regulates the immune response, and wherein the third binding domain upon binding to the third target molecule (e.g., TIGIT, TIM3, LAG3, CTLA4, CD16A, HER2, Nectin-4, Trop2, or CLDN18.2, or variants thereof) localize the immunomodulatory molecule to a tumor microenvironment. See, e.g., FIG. 12C.

In some embodiments, there is provided an immunomodulatory molecule comprising: i) a first antigen-binding polypeptide comprising from N-terminus to C-terminus: a third binding domain (e.g., Fab comprising a VH and an optional CH1), a first hinge region, and a first subunit of an Fc domain or portion thereof, and ii) a second antigen-binding polypeptide comprising from N-terminus to C-terminus: a second binding domain (e.g., PD-L2 or PD-L1 or variant thereof), a first binding domain (e.g., a p35 subunit and a p40 subunit of an IL-12 or variant thereof connected in tandem), a second hinge region, and a second subunit of the Fc domain or portion thereof, wherein the first binding domain upon binding to the first target molecule (e.g., IL-12 receptor) up-regulates an immune response, wherein the second binding domain specifically recognizes a second target molecule (e.g., PD-1), wherein the third binding domain specifically recognizes a third target molecule (e.g. TIGIT, TIM3, LAG3, CTLA4, CD16A, HER2, Nectin-4, Trop2, or CLDN18.2, or variants thereof), wherein the first binding domain upon binding to the first target molecule (e.g., IL-12 receptor) up-regulates an immune response, and wherein the second binding domain upon binding to the second target molecule (e.g., PD-1) down-regulates the immune response, and wherein the third binding domain upon binding to the third target molecule (e.g., TIGIT, TIM3, LAG3, CTLA4, CD16A, HER2, Nectin-4, Trop2, or CLDN18.2) localize the immunomodulatory molecule to a tumor microenvironment. See, e.g., FIG. 12D.

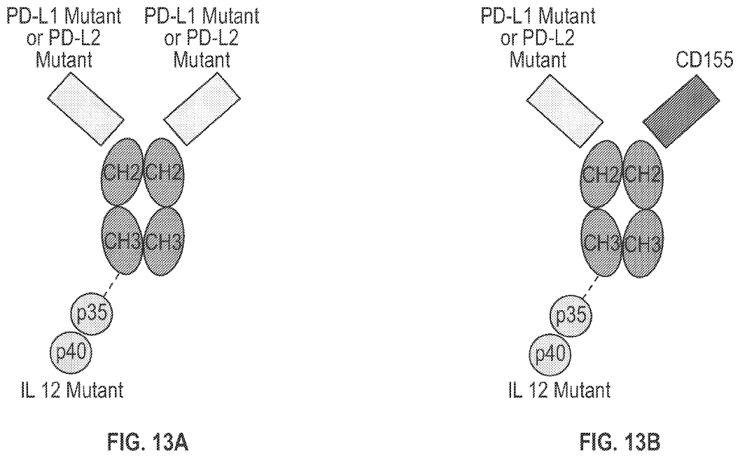

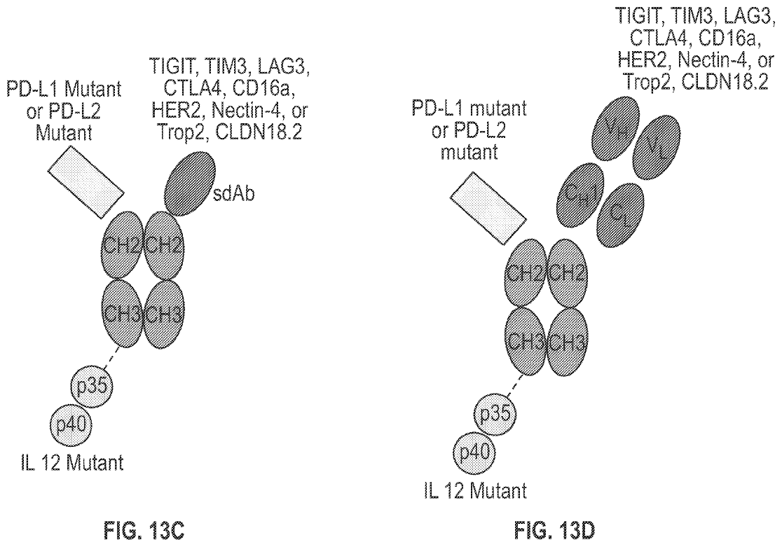

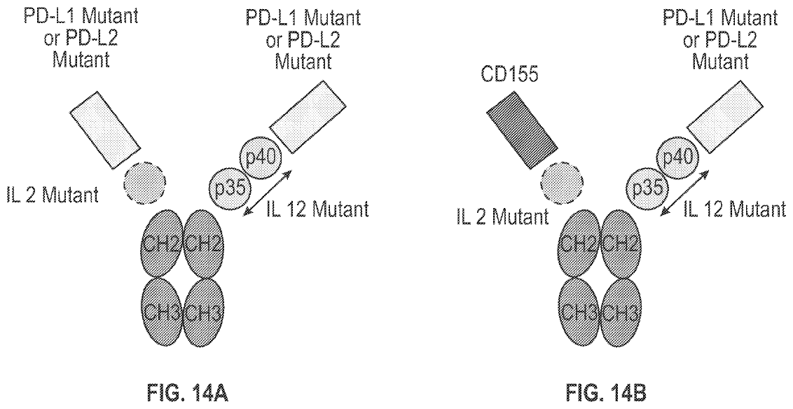

In some embodiments, there is provided an immunomodulatory molecule comprising: i) a first antigen-binding polypeptide comprising from N-terminus to C-terminus: a first second binding domain (e.g., PD-L2 or PD-L1 or variant thereof) positioned at the first hinge region, and a first subunit of an Fc domain or portion thereof, and a first binding domain (e.g., a p35 subunit and a p40 subunit of an IL-12 or variant thereof connected in tandem); and ii) a second antigen-binding polypeptide comprising from N-terminus to C-terminus: a second second binding domain (e.g., PD-L2 or PD-L1 or variant thereof), a second hinge region, and a second subunit of the Fc domain or portion thereof, wherein the first binding domain specifically recognizes a first target molecule (e.g., IL-12 receptor), wherein the first and second second binding domain specifically recognizes a second target molecule (e.g., PD-1), wherein the first binding domain upon binding to the first target molecule (e.g., IL-12 receptor) up-regulates an immune response, and wherein the first and/or second second binding domain upon binding to the second target molecule (e.g., PD-1) down-regulates the immune response. See, e.g., FIG. 13A. In some embodiments, the first and/or second second binding domains are the same. In some embodiments, the first and/or second second binding domains are different. In some embodiments, the first and/or second second binding domain specifically recognize the same epitope. In some embodiments, the first and/or second second binding domain specifically recognize different epitopes.

In some embodiments, there is provided an immunomodulatory molecule comprising: i) a first antigen-binding polypeptide comprising from N-terminus to C-terminus: a first second binding domain (e.g., CD155 or variant thereof) positioned at the first hinge region, and a first subunit of an Fc domain or portion thereof; and ii) a second antigen-binding polypeptide comprising from N-terminus to C-terminus: a second second binding domain (e.g., PD-L2 or PD-L1 or variant thereof), a second hinge region, and a second subunit of the Fc domain or portion thereof, and a first binding domain (e.g., a p35 subunit and a p40 subunit of an IL-12 or variant thereof connected in tandem), wherein the first binding domain specifically recognizes a first target molecule (e.g., IL-12 receptor), wherein the first second binding domain specifically recognizes a second target molecule (e.g., TIGIT), wherein the second second binding domain specifically recognizes a third target molecule (e.g. PD-1), wherein the first binding domain upon binding to the first target molecule (e.g., IL-12 receptor) up-regulates an immune response, and wherein the first and/or second second binding domain upon binding to the second target molecule (e.g., TIGIT and/or PD-1) down-regulates the immune response. See, e.g., FIG. 13B.

In some embodiments, there is provided an immunomodulatory molecule comprising: i) a first antigen-binding polypeptide comprising from N-terminus to C-terminus: a second binding domain (e.g., PD-L2 or PD-L1 or variant thereof) positioned at the first hinge region, and a first subunit of an Fc domain or portion thereof, and a first binding domain (e.g., a p35 subunit and a p40 subunit of an IL-12 or variant thereof connected in tandem); and ii) a second antigen-binding polypeptide comprising from N-terminus to C-terminus: a third binding domain (e.g. a sdAb), a second hinge region, and a second subunit of an Fc domain or portion thereof, wherein the first binding domain upon binding to the first target molecule (e.g., IL-12 receptor) up-regulates an immune response, wherein the second binding domain specifically recognizes a second target molecule (e.g., PD-1), wherein the third binding domain specifically recognizes a third target molecule (e.g. TIGIT, TIM3, LAG3, CTLA4, CD16A, HER2, Nectin-4, Trop2, or CLDN18.2, or variants thereof), wherein the first binding domain upon binding to the first target molecule (e.g., IL-12 receptor) up-regulates an immune response, and wherein the second binding domain upon binding to the second target molecule (e.g., PD-1) down-regulates the immune response, and wherein the third binding domain upon binding to the third target molecule (e.g., TIGIT, TIM3, LAG3, CTLA4, CD16A, HER2, Nectin-4, Trop2, or CLDN18.2) localize the immunomodulatory molecule to a tumor microenvironment. See, e.g., FIG. 13C.

In some embodiments, there is provided an immunomodulatory molecule comprising: i) a first antigen-binding polypeptide comprising from N-terminus to C-terminus: a second binding domain (e.g., PD-L2 or PD-L1 or variant thereof) positioned at the first hinge region, and a first subunit of an Fc domain or portion thereof, and a first binding domain (e.g., a p35 subunit and a p40 subunit of an IL-12 or variant thereof connected in tandem); and ii) a second antigen-binding polypeptide comprising from N-terminus to C-terminus: a third binding domain (e.g., Fab comprising a V_{H} and an optional CH1), a second hinge region, and a second subunit of an Fc domain or portion thereof, wherein the first binding domain upon binding to the first target molecule (e.g., IL-12 receptor) up-regulates an immune response, wherein the second binding domain specifically recognizes a second target molecule (e.g., PD-1), wherein the third binding domain specifically recognizes a third target molecule (e.g. TIGIT, TIM3, LAG3, CTLA4, CD16A, HER2, Nectin-4, Trop2, or CLDN18.2, or variants thereof), wherein the first binding domain upon binding to the first target molecule (e.g., IL-12 receptor) up-regulates an immune response, and wherein the second binding domain upon binding to the second target molecule (e.g., PD-1) down-regulates the immune response, and wherein the third binding domain upon binding to the third target molecule (e.g., TIGIT, TIM3, LAG3, CTLA4, CD16A, HER2, Nectin-4, Trop2, or CLDN18.2) localize the immunomodulatory molecule to a tumor microenvironment. See, e.g., FIG. 13D.

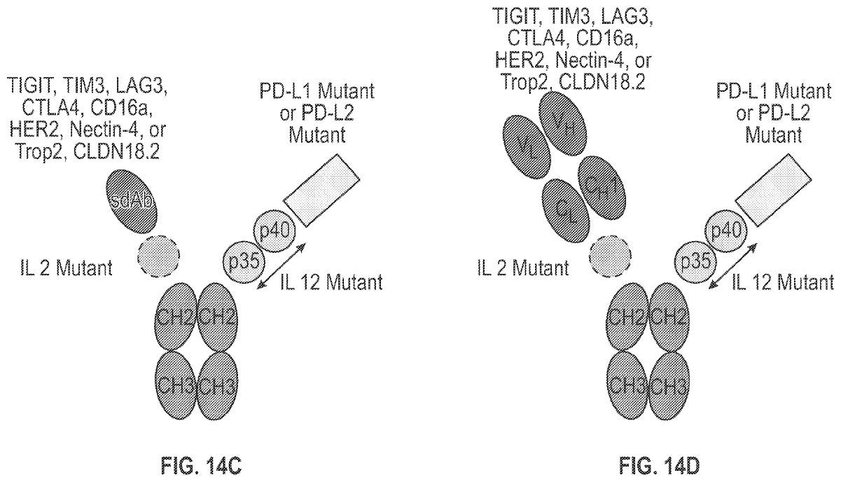

In some embodiments, there is provided an immunomodulatory molecule comprising: i) a first antigen-binding polypeptide comprising from N-terminus to C-terminus: a first second binding domain (e.g., PD-L2 or PD-L1 or variant thereof), a first first binding domain (e.g. IL-2 or variant thereof), a first hinge region, a first subunit of an Fc domain or portion thereof; and ii) a second antigen-binding polypeptide comprising from N-terminus to C-terminus: a second second binding domain (e.g., PD-L2 or PD-L1 or variant thereof), a second first binding domain (e.g., a p35 subunit and a p40 subunit of an IL-12 or variant thereof connected in tandem), a second hinge region, and a second subunit of the Fc domain or portion thereof, wherein the first first binding domain specifically recognizes a first target molecule (e.g., IL-2 receptor), wherein the second first binding domain specifically recognizes a second target molecule (e.g. IL-12 receptor), wherein the first and second second binding domain specifically recognizes a third target molecule (e.g., PD-1), wherein the first first binding domain upon binding to the first or target molecule (e.g., IL-2 receptor) up-regulates an immune response, wherein the second first binding domain upon binding to the second target molecule (e.g., IL-12 receptor) up-regulates an immune response, and wherein the first and/or second second binding domain upon binding to the third target molecule (e.g., PD-1) down-regulates the immune response. See, e.g., FIG. 14A. In some embodiments, the first and/or second first binding domains are the same. In some embodiments, the first and/or second first binding domains are different. In some embodiments, the first and/or second first binding domain specifically recognize the same epitope. In some embodiments, the first and/or second first binding domain specifically recognize different epitopes. In some embodiments, the first and/or second second binding domains are the same. In some embodiments, the first and/or second second binding domains are different. In some embodiments, the first and/or second second binding domain specifically recognize the same epitope. In some embodiments, the first and/or second second binding domain specifically recognize different epitopes.

In some embodiments, there is provided an immunomodulatory molecule comprising: i) a first antigen-binding polypeptide comprising from N-terminus to C-terminus: a first second binding domain (e.g., CD155 or variant thereof), a first first binding domain (e.g. TL-2 or variant thereof), a first hinge region, a first subunit of an Fc domain or portion thereof; and ii) a second antigen-binding polypeptide comprising from N-terminus to C-terminus: a second second binding domain (e.g., PD-L2 or PD-L1 or variant thereof), a second first binding domain (e.g., a p35 subunit and a p40 subunit of an IL-12 or variant thereof connected in tandem), a second hinge region, and a second subunit of the Fc domain or portion thereof, wherein the first first binding domain specifically recognizes a first target molecule (e.g., IL-2 receptor), wherein the second first binding domain specifically recognizes a second target molecule (e.g. IL-12 receptor), wherein the first second binding domain specifically recognizes a third target molecule (e.g., TIGIT), wherein the second second binding domain specifically recognizes a fourth target molecule (e.g. PD-1), wherein the first first binding domain upon binding to the first or target molecule (e.g., IL-2 receptor) up-regulates an immune response, wherein the second first binding domain upon binding to the second target molecule (e.g., IL-12 receptor) up-regulates an immune response, wherein the first second binding domain upon binding to the third target molecule (e.g. TIGIT) down-regulates the immune response, and wherein the second second binding domain upon binding to the fourth target molecule (e.g., PD-1) down-regulates the immune response. See, e.g., FIG. 14B.

In some embodiments, there is provided an immunomodulatory molecule comprising: i) a first antigen-binding polypeptide comprising from N-terminus to C-terminus: a third binding domain (e.g., a sdAb), a first first binding domain (e.g. IL-2 or variant thereof), a first hinge region, a first subunit of an Fc domain or portion thereof; and ii) a second antigen-binding polypeptide comprising from N-terminus to C-terminus: a second binding domain (e.g., PD-L2 or PD-L1 or variant thereof), a second first binding domain (e.g., a p35 subunit and a p40 subunit of an IL-12 or variant thereof connected in tandem), a second hinge region, and a second subunit of the Fc domain or portion thereof, wherein the first first binding domain specifically recognizes a first target molecule (e.g., IL-2 receptor), wherein the second first binding domain specifically recognizes a second target molecule (e.g. IL-12 receptor), wherein the second binding domain specifically recognizes a third target molecule (e.g., PD-1), wherein the third binding domain specifically recognizes a fourth target molecule (e.g., TIGIT, TIM3, LAG3, CTLA4, CD16A, HER2, Nectin-4, Trop2, or CLDN18.2) wherein the first first binding domain upon binding to the first or target molecule (e.g., IL-2 receptor) up-regulates an immune response, wherein the second first binding domain upon binding to the second target molecule (e.g., IL-12 receptor) up-regulates an immune response, wherein the second binding domain upon binding to the third target molecule (e.g., PD-1) down-regulates the immune response, and wherein the third binding domain upon binding to the fourth target molecule (e.g., TIGIT, TIM3, LAG3, CTLA4, CD16A, HER2, Nectin-4, Trop2, or CLDN18.2) localize the immunomodulatory molecule to a tumor microenvironment. See, e.g., FIG. 14C.

In some embodiments, there is provided an immunomodulatory molecule comprising: i) a first antigen-binding polypeptide comprising from N-terminus to C-terminus: a third binding domain (e.g., a Fab comprising a VH and an optional CH1), a first first binding domain (e.g. IL-2 or variant thereof), a first hinge region, a first subunit of an Fc domain or portion thereof; and ii) a second antigen-binding polypeptide comprising from N-terminus to C-terminus: a second binding domain (e.g., PD-L2 or PD-L1 or variant thereof), a second first binding domain (e.g., a p35 subunit and a p40 subunit of an IL-12 or variant thereof connected in tandem), a second hinge region, and a second subunit of the Fc domain or portion thereof, wherein the first first binding domain specifically recognizes a first target molecule (e.g., IL-2 receptor), wherein the second first binding domain specifically recognizes a second target molecule (e.g. IL-12 receptor), wherein the second binding domain specifically recognizes a third target molecule (e.g., PD-1), wherein the third binding domain specifically recognizes a fourth target molecule (e.g., TIGIT, TIM3, LAG3, CTLA4, CD16A, HER2, Nectin-4, Trop2, or CLDN18.2) wherein the first first binding domain upon binding to the first or target molecule (e.g., IL-2 receptor) up-regulates an immune response, wherein the second first binding domain upon binding to the second target molecule (e.g., IL-12 receptor) up-regulates an immune response, wherein the second binding domain upon binding to the third target molecule (e.g., PD-1) down-regulates the immune response, and wherein the third binding domain upon binding to the fourth target molecule (e.g., TIGIT, TIM3, LAG3, CTLA4, CD16A, HER2, Nectin-4, Trop2, or CLDN18.2) localize the immunomodulatory molecule to a tumor microenvironment. See, e.g., FIG. 14D.

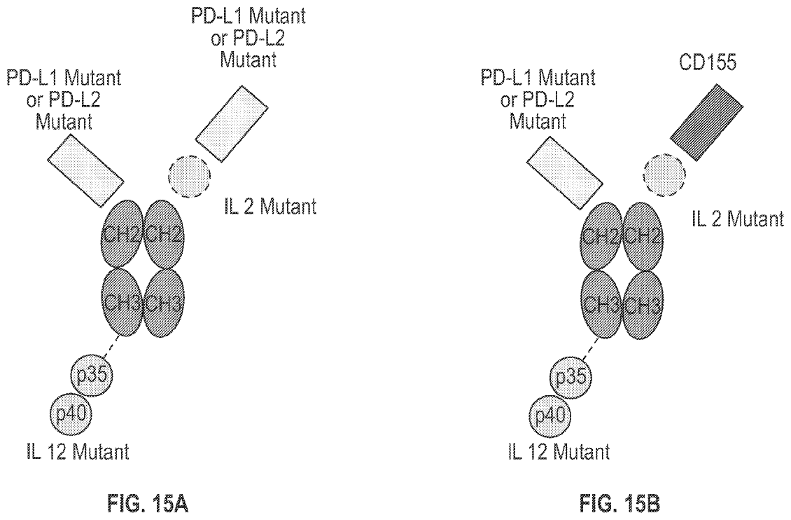

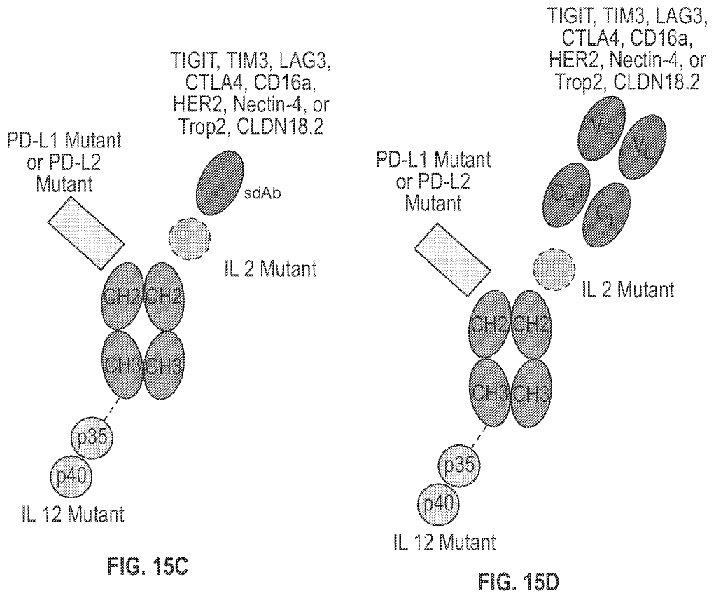

In some embodiments, there is provided an immunomodulatory molecule comprising: i) a first antigen-binding polypeptide comprising from N-terminus to C-terminus: a first second binding domain (e.g., PD-L2 or PD-L1 or variant thereof) positioned at the first hinge region, and a first subunit of an Fc domain or portion thereof, and a first first binding domain (e.g., a p35 subunit and a p40 subunit of an IL-12 or variant thereof connected in tandem); and ii) a second antigen-binding polypeptide comprising from N-terminus to C-terminus: a second second binding domain (e.g., PD-L2 or PD-L1 or variant thereof), a second first binding domain (e.g., IL-2 or variant thereof), a second hinge region, and a second subunit of the Fc domain or portion thereof, wherein the first first binding domain specifically recognizes a first target molecule (e.g., IL-12 receptor), wherein the second first binding domain specifically recognizes a second target molecule (e.g. IL-2 receptor), wherein the first and second second binding domain specifically recognizes a third target molecule (e.g., PD-1), wherein the first first binding domain upon binding to the first target molecule (e.g., IL-12 receptor) up-regulates an immune response, wherein the second first binding domain upon binding to the second target molecule (e.g., IL-2 receptor) up-regulates an immune response, and wherein the first and/or second second binding domain upon binding to the third target molecule (e.g., PD-1) down-regulates the immune response. See, e.g., FIG. 15A. In some embodiments, the first and/or second first binding domains are the same. In some embodiments, the first and/or second first binding domains are different. In some embodiments, the first and/or second first binding domain specifically recognize the same epitope. In some embodiments, the first and/or second first binding domain specifically recognize different epitopes. In some embodiments, the first and/or second second binding domains are the same. In some embodiments, the first and/or second second binding domains are different. In some embodiments, the first and/or second second binding domain specifically recognize the same epitope. In some embodiments, the first and/or second second binding domain specifically recognize different epitopes.

In some embodiments, there is provided an immunomodulatory molecule comprising: i) a first antigen-binding polypeptide comprising from N-terminus to C-terminus: a first second binding domain (e.g., PD-L2 or PD-L1 or variant thereof) positioned at the first hinge region, and a first subunit of an Fc domain or portion thereof, and a first first binding domain (e.g., a p35 subunit and a p40 subunit of an IL-12 or variant thereof connected in tandem); and ii) a second antigen-binding polypeptide comprising from N-terminus to C-terminus: a second second binding domain (e.g., CD155 or variant thereof), a second first binding domain (e.g., IL-2 or variant thereof), a second hinge region, and a second subunit of the Fc domain or portion thereof, wherein the first first binding domain specifically recognizes a first target molecule (e.g., IL-12 receptor), wherein the second first binding domain specifically recognizes a second target molecule (e.g. IL-2 receptor), wherein the first second binding domain specifically recognizes a third target molecule (e.g., PD-1), wherein the second second binding domain recognizes a fourth target molecule (e.g., TIGIT), wherein the first first binding domain upon binding to the first target molecule (e.g., IL-12 receptor) up-regulates an immune response, wherein the second first binding domain upon binding to the second target molecule (e.g., IL-2 receptor) up-regulates an immune response, wherein the first second binding domain upon binding to the third target molecule (e.g., PD-1) down-regulates the immune response, and wherein the second second binding domain upon binding to the third target molecule (e.g., TIGIT) down regulates the immune response. See, e.g., FIG. 15B.

In some embodiments, there is provided an immunomodulatory molecule comprising: i) a first antigen-binding polypeptide comprising from N-terminus to C-terminus: a first second binding domain (e.g., PD-L2 or PD-L1 or variant thereof) positioned at the first hinge region, and a first subunit of an Fc domain or portion thereof, and a first first binding domain (e.g., a p35 subunit and a p40 subunit of an IL-12 or variant thereof connected in tandem); and ii) a second antigen-binding polypeptide comprising from N-terminus to C-terminus: a third binding domain (e.g., a sdAb), a second first binding domain (e.g., IL-2 or variant thereof), a second hinge region, and a second subunit of an Fc domain or portion thereof, wherein the first first binding domain specifically recognizes a first target molecule (e.g., IL-12 receptor), wherein the second first binding domain specifically recognizes a second target molecule (e.g. IL-2 receptor), wherein the second binding domain specifically recognizes a third target molecule (e.g., PD-1), wherein the third binding domain specifically recognizes a fourth target molecule (e.g., TIGIT, TIM3, LAG3, CTLA4, CD16A, HER2, Nectin-4, Trop2, or CLDN18.2) wherein the first first binding domain upon binding to the first or target molecule (e.g., IL-12 receptor) up-regulates an immune response, wherein the second first binding domain upon binding to the second target molecule (e.g., IL-2 receptor) up-regulates an immune response, wherein the second binding domain upon binding to the third target molecule (e.g., PD-1) down-regulates the immune response, and wherein the third binding domain upon binding to the fourth target molecule (e.g., TIGIT, TIM3, LAG3, CTLA4, CD16A, HER2, Nectin-4, Trop2, or CLDN18.2) localize the immunomodulatory molecule to a tumor microenvironment. See, e.g., FIG. 15C.

In some embodiments, there is provided an immunomodulatory molecule comprising: i) a first antigen-binding polypeptide comprising from N-terminus to C-terminus: a first second binding domain (e.g., PD-L2 or PD-L1 or variant thereof) positioned at the first hinge region, and a first subunit of an Fc domain or portion thereof, and a first first binding domain (e.g., a p35 subunit and a p40 subunit of an IL-12 or variant thereof connected in tandem); and ii) a second antigen-binding polypeptide comprising from N-terminus to C-terminus: a third binding domain (e.g., a Fab comprising a VH and an optional CH1), a second first binding domain (e.g., IL-2 or variant thereof), a second hinge region, and a second subunit of an Fc domain or portion thereof, wherein the first first binding domain specifically recognizes a first target molecule (e.g., IL-12 receptor), wherein the second first binding domain specifically recognizes a second target molecule (e.g. IL-2 receptor), wherein the second binding domain specifically recognizes a third target molecule (e.g., PD-1), wherein the third binding domain specifically recognizes a fourth target molecule (e.g., TIGIT, TIM3, LAG3, CTLA4, CD16A, HER2, Nectin-4, Trop2, or CLDN18.2) wherein the first first binding domain upon binding to the first or target molecule (e.g., IL-12 receptor) up-regulates an immune response, wherein the second first binding domain upon binding to the second target molecule (e.g., IL-2 receptor) up-regulates an immune response, wherein the second binding domain upon binding to the third target molecule (e.g., PD-1) down-regulates the immune response, and wherein the third binding domain upon binding to the fourth target molecule (e.g., TIGIT, TIM3, LAG3, CTLA4, CD16A, HER2, Nectin-4, Trop2, or CLDN18.2) localize the immunomodulatory molecule to a tumor microenvironment. See, e.g., FIG. 15D.

In some embodiments, there is provided an immunomodulatory molecule as described in any of FIGS. 1A-1W and 11A-15D, Examples, and Sequence Listing herein.

### Binding Domains Specifically Recognizing Target Molecules

The immunomodulatory molecules described herein comprise a first binding domain specifically recognizing a first target molecule and a second binding domain specifically recognizing a second target molecule, wherein the first binding domain upon binding to the first target molecule up-regulates an immune response, and wherein the second binding domain upon binding to the second target molecule down-regulates the immune response.

In some embodiments, the first binding domain upon binding to the first target molecule up-regulates the immune response by an activity (“up-regulated activity”) selected from one or more of up-regulating release of an immunostimulatory cytokine, down-regulating release of an immunosuppressive cytokine, up-regulating immune cell proliferation, up-regulating immune cell differentiation, up-regulating immune cell activation, up-regulating cytotoxicity against a tumor cell, and up-regulating elimination of an infectious agent.

In some embodiments, the second binding domain upon binding to the second target molecule down-regulates the immune response by an activity (“down-regulated activity”) selected from one or more of down-regulating release of an immunostimulatory cytokine, up-regulating release of an immunosuppressive cytokine, down-regulating immune cell proliferation, down-regulating immune cell differentiation, down-regulating immune cell activation, down-regulating cytotoxicity against a tumor cell, and down-regulating elimination of an infectious agent.

In some embodiments, the first binding domain upon binding to the first target molecule, and the second binding domain upon binding to the second target molecule, modulate (e.g., modulate at least about any of 10%, 20%, 30%, 40%, 50%, 60%, 70%, 80%, 90%, 100%, or more) the immune response by an activity independently selected from one or more of cytokine release, immune cell proliferation, immune cell differentiation, immune cell activation, cytotoxicity against a tumor cell, and up-regulating elimination of an infectious agent. For example, in some embodiments, the first binding domain upon binding to the first target molecule up-regulates (e.g., up-regulates (or down-regulates in the case of release of an immunosuppressive cytokine) at least about any of 10%, 20%, 30%, 40%, 50%, 60%, 70%, 80%, 90%, 100%, or more) the immune response by an activity (“up-regulated activity”) selected from one or more of up-regulating release of an immunostimulatory cytokine, down-regulating release of an immunosuppressive cytokine, up-regulating immune cell proliferation, up-regulating immune cell differentiation, up-regulating immune cell activation, up-regulating cytotoxicity against a tumor cell, and up-regulating elimination of an infectious agent. In some embodiments, the second binding domain upon binding to the second target molecule down-regulates (e.g., down-regulates (or up-regulates in the case of release of an immunosuppressive cytokine) at least about any of 10%, 20%, 30%, 40%, 50%, 60%, 70%, 80%, 90%, or 100%) the immune response by an activity (“down-regulated activity”) selected from one or more of down-regulating release of an immunostimulatory cytokine, up-regulating release of an immunosuppressive cytokine, down-regulating immune cell proliferation, down-regulating immune cell differentiation, down-regulating immune cell activation, down-regulating cytotoxicity against a tumor cell, and down-regulating elimination of an infectious agent. In some embodiments, the immunostimulatory cytokine is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF. In some embodiments, the immunosuppressive cytokine is selected from the group consisting of IL-1Ra, IL-4, IL-5, IL-6, IL-10, IL-11, IL-13, IL-27, IL-33, IL-35, IL-37, IL-39, IFN-α, LIF, and TGF-β.

In some embodiments, the first target molecule and/or the second target molecule is a stimulatory checkpoint molecule. In some embodiments, the stimulatory checkpoint molecule is selected from the group consisting of CD27, CD28, CD40, CD122, CD137, OX40, GITR, and ICOS. In some embodiments, the first binding domain is an agonist antibody or antigen-binding fragment thereof. In some embodiments, the agonist ligand is selected from the group consisting of CD27L (TNFSF7, CD70), CD40L (CD154), CD80, CD86, CD137L, OX40L (CD252), GITRL, and ICOSLG (CD275). In some embodiments, the first binding domain is a variant of an agonist ligand, and wherein the variant of the agonist ligand has increased (e.g., increase at least about any of 10%, 20%, 30%, 40%, 50%, 60%, 70%, 80%, 90%, 100% or more) activity (e.g., binding affinity and/or biological activity) to the first target molecule compared to the agonist ligand. In some embodiments, the first binding domain is a variant of an agonist ligand, and wherein the variant of the agonist ligand has decreased (e.g., decrease at least about any of 10%, 20%, 30%, 40%, 50%, 60%, 70%, 80%, 90%, or 100%) activity (e.g., binding affinity and/or biological activity) to the first target molecule compared to the agonist ligand. In some embodiments, the second binding domain is an antagonist antibody or antigen-binding fragment thereof (e.g., VH, VHH, scFv, Fab, full-length Ab). In some embodiments, the second binding domain is an antagonist ligand or variant thereof. In some embodiments, the second binding domain is a variant of an antagonist ligand, and wherein the variant of the antagonist ligand has increased (e.g., increase at least about any of 10%, 20%, 30%, 40%, 50%, 60%, 70%, 80%, 90%, 100% or more) activity (e.g., binding affinity and/or biological activity) to the second target molecule compared to the antagonist ligand. In some embodiments, the second binding domain is a variant of an antagonist ligand, and wherein the variant of the antagonist ligand has decreased (e.g., decrease at least about any of 10%, 20%, 30%, 40%, 500%, 60%, 70%, 80%, 90%, or 100%) activity (e.g., binding affinity and/or biological activity) to the second target molecule compared to the antagonist ligand.

In some embodiments, the first target molecule and/or the second target molecule is a receptor of an immunostimulatory cytokine. In some embodiments, the immunostimulatory cytokine is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF. In some embodiments, the first binding domain is the immunostimulatory cytokine or variant thereof. In some embodiments, the first binding domain is a variant of an immunostimulatory cytokine, and wherein the variant of the immunostimulatory cytokine has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the first target molecule compared to the immunostimulatory cytokine. In some embodiments, the first binding domain is IL-2 or variant thereof. In some embodiments, the first binding domain is an IL-2 variant that has decreased (e.g., decrease at least about any of 10%, 20%, 30%, 40%, 50%, 60%, 70%, 80%, 90%, or 100%) activity (e.g., binding affinity and/or biological activity) to IL-2 receptor compared to a wild-type IL-2. In some embodiments, the first binding domain is IL-12 or variant thereof. In some embodiments, the first binding domain is an IL-12 variant that has decreased (e.g., decrease at least about any of 10%, 20%, 30%, 40%, 50%, 60%, 70%, 80%, 90%, or 100%) activity (e.g., binding affinity and/or biological activity) to IL-12 receptor compared to a wild-type IL-12. In some embodiments, the first binding domain is an agonist antibody or antigen-binding fragment thereof (e.g., VH, VHH, scFv, Fab, full-length Ab, such as an agonist of IL-12 receptor signaling). In some embodiments, the second binding domain is an antagonist antibody or antigen-binding fragment thereof (e.g., VII, V_{H}H, scFv, Fab, full-length Ab). In some embodiments, the second binding domain is antagonist ligand or variant thereof (e.g., blocks or reduces IL-12 receptor signaling). In some embodiments, the second binding domain is a variant of an antagonist ligand, and wherein the variant of the antagonist ligand has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the second target molecule compared to the antagonist ligand.

The receptor of IL-2, interleukin-2 receptor (IL-2R), is a heterotrimeric protein expressed on the surface of certain immune cells, such as lymphocytes. IL-2R has three forms generated by different combinations of a chain (IL-2Rα, CD25, Tac antigen), β chain (IL-2Rβ, CD122), and γ chain (IL-2Rγ, γ_{c}, common gamma chain, or CD132). IL-2Rα binds IL-2 with low affinity, and the complex of IL-2Rβ and IL-2Rγ binds IL-2 with intermediate affinity, primarily on memory T cells and NK cells. The complex of all α, β, and γ chains bind IL-2 with high affinity on activated T cells and regulatory T cells (Tregs). CD25 (IL-2Rα) plays a critical role in the development and maintenance of Tregs, and may play a role in Treg expression of CD62L, which is required for the entry of Tregs into lymph nodes (Malek and Bayer, 2004). CD25 is a marker for activated T cells and Treg. Experimental data suggested an immunosuppressive capacity of antagonist anti-CD25 that significantly delayed rejection of heart allografts in the mouse (Kirkman et al., 1985) and of renal allografts in nonhuman primates (Reed et al., 1989). Exemplary antagonist anti-CD25 antibodies include, but are not limited to basiliximab (e.g., Simulect®), daclizumab (e.g., Zinbryta®).

In some embodiments, the first target molecule and/or the second target molecule is an activating immune cell surface receptor. In some embodiments, the activating immune cell surface receptor is selected from the group consisting of CD2, CD3, CD4, CD8, CD16, CD56, CD96, CD161, CD226, NKG2C, NKG2D, NKG2E, NKG2F, NKG2H, NKp30, NKp44, NKp46, CD11c, CD11b, CD13, CD45RO, CD33, CD123, CD62L, CD45RA, CD36, CD163, and CD206. In some embodiments, the first binding domain is an agonist antibody or antigen-binding fragment thereof (e.g., VH, VHH, scFv, Fab, full-length Ab). In some embodiments, the first binding domain is an agonist ligand or variant thereof. In some embodiments, the first binding domain is a variant of an agonist ligand, and wherein the variant of the agonist ligand has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the first target molecule compared to the agonist ligand. In some embodiments, the second binding domain is an antagonist antibody or antigen-binding fragment thereof (e.g., VH, VHH, scFv, Fab, full-length Ab). In some embodiments, the second binding domain is an antagonist ligand or variant thereof. In some embodiments, the second binding domain is a variant of an antagonist ligand, and wherein the variant of the antagonist ligand has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the second target molecule compared to the antagonist ligand.

In some embodiments, the first target molecule and/or the second target molecule is an inhibitory checkpoint molecule. In some embodiments, the inhibitory checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, Siglec7, Siglec9, and VISTA. In some embodiments, the first binding domain is an antagonist ligand or variant thereof (e.g., blocks or reduces PD-1 signaling). In some embodiments, the first binding domain is an antagonist ligand or variant thereof of PD-1. In some embodiments, the first binding domain is a variant of an antagonist ligand, and wherein the variant of the antagonist ligand has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the first target molecule compared to the antagonist ligand. In some embodiments, the first binding domain is an antagonist antibody or antigen-binding fragment thereof (e.g., VH, VHH, scFv, Fab, full-length Ab). In some embodiments, the first binding domain is an antagonist anti-PD-1 antibody or antigen-binding fragment thereof. In some embodiments, the second binding domain is an agonist antibody or antigen-binding fragment thereof (e.g., VH, VHH, scFv, Fab, full-length Ab). In some embodiments, the agonist antibody or antigen-binding fragment thereof specifically recognizes PD-1, TIGIT, LAG-3, TIM-3, or CTLA-4. In some embodiments, the second binding domain is an agonist ligand or variant thereof. In some embodiments, the second target molecule is PD-1, and the second binding domain is PD-L1, PD-L2, or variant thereof. In some embodiments, the second target molecule is TIGIT, and the second binding domain is CD112 (PVRL2, nectin-2), CD155 (PVR), or variant thereof. In some embodiments, the second target molecule is LAG-3, and wherein the second binding domain is MHC II, LSECtin, or variant thereof. In some embodiments, the second target molecule is TIM-3, and wherein the second binding domain is Galectin-9, Caecam-1, HMGB-1, phosphatidylserine, or variant thereof. In some embodiments, the second target molecule is CTLA-4, and wherein the second binding domain is CD80, CD86, or variant thereof. In some embodiments, the second binding domain is a variant of an agonist ligand, and wherein the variant of the agonist ligand has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the second target molecule compared to the agonist ligand. In some embodiments, the second binding domain is a variant of PD-L1 (or PD-L2), that has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to PD-1 compared to the wild-type PD-L1 (or PD-L2). In some embodiments, the second binding domain comprises an extracellular domain of the agonist ligand or variant thereof.

PD-1 (programmed cell death protein 1) is a part of the B7/CD28 family of co-stimulatory molecules that regulate T-cell activation and tolerance, and thus antagonistic anti-PD-1 antibodies or PD-1 ligand-Fc fusion protein can be useful for overcoming tolerance. PD-1 has been defined as a receptor for B7-4. B7-4 can inhibit immune cell activation upon binding to an inhibitory receptor on an immune cell. Engagement of the PD-1/PD-L1 pathway results in inhibition of T-cell effector function, cytokine secretion and proliferation. (Turnis et al., OncoImmunology 1(7):1172-1174, 2012). High levels of PD-1 are associated with exhausted or chronically stimulated T cells. Moreover, increased PD-1 expression correlates with reduced survival in cancer patients. Agents for down modulating PD-1, B7-4, and the interaction between B7-4 and PD-1 inhibitory signal in an immune cell can result in enhancement of the immune response. Exemplary antagonist anti-PD-1 antibodies include, but are not limited to, pembrolizumab (e.g., Keytruda®), cemiplimab (Libtayo®), and nivolumab (e.g., Opdivo®).

In some embodiments, the second binding domain comprises an anti-PD-1 antibody fragment derived from nivolumab (antagonist). In some embodiments, the anti-PD-1 antibody fragment comprises VH-CDR1 and VH-CDR2, and VH-CDR3 of a VH comprising the sequence of SEQ ID NO: 48, and VL-CDR1, VL-CDR2, and VL-CDR3 of a VL comprising the sequence of SEQ ID NO: 49. In some embodiments, VH-CDR3 further comprises any one of the following mutations relative to SEQ ID NO: 48: D100N, D100G, D100R, N99G, N99A, or N99M. In some embodiments, the anti-PD-1 antibody fragment comprising such VH-CDR3 mutations have reduced binding affinity (e.g., reduced at least about 2, 3, 4, 5, 6, 7, 8, 9, 10, 20, 50, 100, 1000, or more fold) to PD-1 compared to nivolumab.

In some embodiments, the second binding domain is an agonist antibody or antigen-binding fragment thereof specifically recognizes PD-1 (“anti-PD-1 agonist antibody or antigen-binding fragment thereof”).

PD-L1 (programmed cell death-ligand 1) is also known as cluster of differentiation 274 (CD274) or B7 homolog 1 (B7-H1). PD-L1 serves as a ligand for PD-1 to play a major role in suppressing the immune system during particular events such as pregnancy, tissue allographs, autoimmune disease and other disease states such as hepatitis and cancer. The formation of PD-1 receptor/PD-L1 ligand complex transmits an inhibitory signal, which reduces the proliferation of CD8⁺ T cells at the lymph nodes. Exemplary antagonist anti-PD-L1 antibodies include, but are not limited to, atezolizumab (e.g., Tecentriq®), avelumab (e.g., Bavencio®), and durvalumab (e.g., IMFINZI™).

In some embodiments, the second binding domain is PD-L1 or variant thereof. In some embodiments, the wt PD-L1 extracellular domain comprises the sequence of SEQ ID NO: 121. In some embodiments, the second binding domain is a PD-L1 variant, and the PD-L1 variant has increased (e.g., increase at least about any of 10%, 20%, 30%, 40%, 50%, 60%, 70%, 80%, 90%, 100% or more) activity (e.g., binding affinity and/or biological activity) to PD-1 compared to a wildtype PD-L1. In some embodiments, the PD-L1 variant comprises one or more mutations at a position selected from the group consisting of 154, Y56, E58, R113, M115, S117, and G119 relative to a wildtype PD-L1 (SEQ ID NO: 120). In some embodiments, the PD-L1 variant comprises one or more mutations selected from the group consisting of I54Q, Y56F, E58M, R113T, M115L, S117A, and G119K relative to a wildtype PD-L1 (SEQ ID NO: 120). In some embodiments, the PD-L1 variant comprises an I54Q/Y56F/E58M/R 113T/MI 15L/S117A/G119K mutation relative to a wildtype PD-L1 (SEQ ID NO: 120). In some embodiments, the mutant PD-L1 extracellular domain comprises the sequence of any one of SEQ ID NOs: 122-129.

PD-L2 (programmed cell death 1 ligand 2, B7-DC, CD273) is another immune checkpoint receptor ligand of PD-1. PD-L2 plays a role in negative regulation of the adaptive immune response. Engagement of PD-1 by PD-L2 dramatically inhibits T cell receptor (TCR)-mediated proliferation and cytokine production by T cells. At low antigen concentrations, PD-L2-PD-1 interactions inhibit strong B7-CD28 signals. In contrast, at high antigen concentrations, PD-L2-PD-1 interactions reduce cytokine production but do not inhibit T cell proliferation.

In some embodiments, the second binding domain is PD-L2 or variant thereof. In some embodiments, the second binding domain is a PD-L2 variant, and the PD-L2 variant has increased (e.g., increase at least about any of 10%, 20%, 30%, 40%, 50%, 60%, 70%, 80%, 90%, 100% or more) activity (e.g., binding affinity and/or biological activity) to PD-1 compared to a wildtype PD-L2.

In some embodiments, the PD-L2 extracellular domain comprises the sequence of SEQ ID NO: 106. In some embodiments, the PD-L2 extracellular domain or portion thereof is derived from wildtype (e.g., wildtype human) PD-L2. In some embodiments, the PD-L2 extracellular domain or portion thereof comprises one or more mutations (e.g., deletion, insertion, or replacement). In some embodiments, the PD-L2 variant comprises one or more mutations at a position selected from the group consisting of T56, S58, and Q60 (e.g., T56V, S58V, Q60L/T56V, S58V/Q60L) relative to a wildtype PD-L2 (SEQ ID NO; 105). In some embodiments, the mutated PD-L2 extracellular domain or portion thereof has increased (such as any of about 2, 3, 4, 5, 10, 50, 100, 100-fold higher) binding affinity to PD-1 compared to wildtype PD-L2 extracellular domain or portion thereof. In some embodiments, the mutant PD-L2 extracellular domain comprises the sequence of any one of SEQ ID NOs: 107-110. In some embodiments, the mutated PD-L2 extracellular domain or portion thereof has reduced (such as any of about 2, 3, 4, 5, 10, 50, 100, 100-fold lower) binding affinity to PD-1 compared to wildtype PD-L2 extracellular domain or portion thereof.

Cytotoxic T-Lymphocyte-Associated protein 4 (CTLA-4, or CD152) is a homolog of CD28, and is known as an inhibitory immune checkpoint molecule upregulated on activated T-cells. CTLA-4 also binds to B7-1 and B7-2, but with greater affinity than CD28. The interaction between B7 and CTLA-4 dampens T cell activation, which constitutes an important mechanism of tumor immune escape. Antagonist anti-CTLA-4 antibody therapy has shown promise in a number of cancers, such as melanoma. Exemplary antagonist anti-CTLA-4 antibodies include, but are not limited to, ipilimumab (e.g., Yervoy®).

In some embodiments, the second binding domain is CD155 (e.g., extracellular domain) or variant thereof. In some embodiments, the extracellular domain of wildtype human CD155 comprises the sequence of SEQ ID NO: 137. CD155 can bind to TIGIT, and down-regulates immune response.

In some embodiments, the first target molecule and/or the second target molecule is a receptor of an immunosuppressive cytokine. In some embodiments, the immunosuppressive cytokine is selected from the group consisting of IL-1 Ra, IL-4, IL-5, TL-6, IL-10, IL-11, IL-13, TL-27, IL-33, IL-35, IFN-α, LIF, and TGF-β. In some embodiments, the second binding domain is the immunosuppressive cytokine or variant thereof. In some embodiments, the second binding domain is a variant of the immunosuppressive cytokine, and the variant of the immunosuppressive cytokine has increased (e.g., increase at least about any of 10%, 20%, 30%/0, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the second target molecule compared to the immunosuppressive cytokine. In some embodiments, the second binding domain is IL-10 or variant thereof. In some embodiments, the second binding domain is TGF-β or variant thereof. In some embodiments, the second binding domain is an agonist antibody or antigen-binding fragment thereof (e.g., VH, VHH, scFv, Fab, full-length Ab). In some embodiments, the first binding domain is an antagonist antibody or antigen-binding fragment thereof (e.g., V_{H}, V_{H}H, scFv, Fab, full-length Ab). In some embodiments, the first binding domain is antagonist ligand or variant thereof. In some embodiments, the first binding domain is a variant of an antagonist ligand, and wherein the variant of the antagonist ligand has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the first target molecule compared to the antagonist ligand.

In some embodiments, the first target molecule and/or the second target molecule is an inhibitory immune cell surface receptor. In some embodiments, the inhibitory immune cell surface receptor is selected from the group consisting of CD5, NKG2A, NKG2B, KLRG1, FCRL4, Siglec2, CD72, CD244, GP49B, Lair-1, PirB, PECAM-1, CD200R, ILT2, and KIR2DL. In some embodiments, the second binding domain is an agonist antibody or antigen-binding fragment thereof (e.g., VH, VHH, scFv, Fab, full-length Ab). In some embodiments, the second binding domain is an agonist ligand or variant thereof. In some embodiments, the second binding domain is a variant of an agonist ligand, wherein the variant of the agonist ligand has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the second target molecule compared to the agonist ligand. In some embodiments, the first binding domain is an antagonist antibody or antigen-binding fragment thereof (e.g., VH, VHH, scFv, Fab, full-length Ab, such as blocks or reduces NKG2B signaling). In some embodiments, the first binding domain is an antagonist ligand or variant thereof. In some embodiments, the first binding domain is a variant of an antagonist ligand, and wherein the variant of the antagonist ligand has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to the first target molecule compared to the antagonist ligand.

In some embodiments, the first binding domain is IL-12 or variant thereof, and the second binding domain is an agonist antibody or antigen-binding fragment thereof (e.g., VH, VHH, scFv, Fab, full-length Ab) specifically recognizing PD-1. Such immunomodulatory molecule is hereinafter also referred to as “IL-12/anti-PD-1 agonist Ab.” In some embodiments, the first binding domain is IL-12 or variant thereof, and wherein the second binding domain is PD-L1 (or extracellular domain thereof) or variant thereof. Such immunomodulatory molecule is hereinafter also referred to as “IL-12/PD-L1 immunomodulatory molecule” or “IL-12/PD-L1 immunocytokine.” In some embodiments, the second binding domain is a variant of PD-L1, and wherein the variant of PD-L1 has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to PD-1 compared to a wild-type PD-L1. In some embodiments, the first binding domain is IL-12 or variant thereof, and wherein the second binding domain is PD-L2 (or extracellular domain thereof) or variant thereof. Such immunomodulatory molecule is hereinafter also referred to as “IL-12/PD-L2 immunomodulatory molecule” or “IL-12/PD-L2 immunocytokine.” In some embodiments, the second binding domain is a variant of PD-L2, and wherein the variant of PD-L2 has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to PD-1 compared to a wild-type PD-L2. In some embodiments, the first binding domain is an IL-12 variant, wherein the TL-12 variant has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to IL-12 receptor compared to a wild-type IL-12.

In some embodiments, the first binding domain is IL-2 or variant thereof, and the second binding domain is an agonist antibody or antigen-binding fragment thereof (e.g., VH, VHH, scFv, Fab, full-length Ab) specifically recognizing PD-1. Such immunomodulatory molecule is hereinafter also referred to as “IL-2/anti-PD-1 agonist Ab.” In some embodiments, the first binding domain is IL-2 or variant thereof, and wherein the second binding domain is PD-L1 (or extracellular domain thereof) or variant thereof. Such immunomodulatory molecule is hereinafter also referred to as “IL-2/PD-L1 immunomodulatory molecule” or “IL-2/PD-L1 immunocytokine.” In some embodiments, the second binding domain is a variant of PD-L1, and wherein the variant of PD-L1 has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to PD-1 compared to a wild-type PD-L1. In some embodiments, the first binding domain is IL-2 or variant thereof, and wherein the second binding domain is PD-L2 (or extracellular domain thereof) or variant thereof. Such immunomodulatory molecule is hereinafter also referred to as “IL-2/PD-L2 immunomodulatory molecule” or “IL-2/PD-L2 immunocytokine.” In some embodiments, the second binding domain is a variant of PD-L2, and wherein the variant of PD-L2 has increased (e.g., increase at least about any of 10%, 20%, 30%, 40%, 50%, 60%, 70%, 80%, 90%, 10% or more) or decreased (e.g., decrease at least about any of 10%, 20%, 30%, 40%, 50%, 60%, 70%, 80%, 90%, or 100%) activity (e.g., binding affinity and/or biological activity) to PD-1 compared to a wild-type PD-L2. In some embodiments, the first binding domain is an IL-2 variant, wherein the IL-2 variant has increased (e.g., increase at least about any of 10%, 20%, 30%, 40%, 50%, 60%, 70%, 80%, 90%, 100% or more) or decreased (e.g., decrease at least about any of 10%, 20%, 30%, 40%, 50%, 60%, 70%, 80%, 90%, or 100%) activity (e.g., binding affinity and/or biological activity) to IL-2 receptor compared to a wild-type IL-2.

In some embodiments, the immunomodulatory molecule further comprises a third binding domain specifically recognizing a third target molecule. In some embodiments, the third binding domain and the second binding domain are the same. In some embodiments, the third binding domain and the second binding domain are different. In some embodiments, the third target molecule and the second target molecule are the same. In some embodiments, the third target molecule and the second target molecule are different.

In some embodiments, the target molecule is a cell surface molecule (e.g., extracellular domain of a receptor/ligand). In some embodiments, the target molecule acts as a cell surface marker on a target cell (e.g., immune cell) associated with a special disease state. The target molecules specifically recognized by the binding domains may be directly or indirectly involved in the diseases.

The binding domains described herein can be of any format known in the art or derived from any suitable antibodies or molecules. In some embodiments, the first binding domain is positioned at a hinge region between a second binding domain and an Fc domain subunit or portion thereof of the immunomodulatory molecule, and the second binding domain is in a format which ensures that the binding of the first binding domain (e.g., immunostimulatory cytokine moiety) to its first target molecule (e.g., cytokine receptor) is reduced in the absence of second target molecule binding of the second binding domain, for example, without second target molecule binding, reducing the activity (e.g., binding affinity to cytokine receptor and/or biological activity) of the first binding domain positioned at the hinge region to be no more than about 70% of that of a corresponding first binding domain (e.g., cytokine or variant thereof) in a free state. For example, the binding domain can be an antigen-binding fragment selected from an scFv, a VH, a VL, an scFv-scFv, an Fv, a Fab, a Fab′, a (Fab′)2, a minibody, a diabody, a domain antibody variant (dAb), a single domain antibody (sdAb) such as a camelid antibody (VHH) or a V_{NAR}, a fibronectin 3 domain variant, an ankyrin repeat variant, and other target molecule-specific binding domains derived from other protein scaffolds. In some embodiments, the antigen-binding fragment is an scFv. In some embodiments, the antigen-binding fragment is a Fab. In some embodiments, the antigen-binding fragment is formed by a V_{H} from a first polypeptide chain and a V_{L} from a second polypeptide chain. In some embodiments, the antigen-binding fragment is human. In some embodiments, the antigen-binding fragment is humanized. In some embodiments, the antigen-binding fragment is chimeric. In some embodiments, the antigen-binding fragment is derived from a monoclonal antibody of mouse, rat, monkey, or rabbit, etc.

In some embodiments, the immunomodulatory molecule comprises two or more first binding domains (e.g., immunostimulatory cytokine moiety). In some embodiments, the immunomodulatory molecule comprises two or more second binding domains (e.g., PD-L1 or PD-L2 extracellular domain, or anti-PD-1 agonist Fab, scFv, sdAb, etc.). In some embodiments, the immunomodulatory molecule further comprises one or more third binding domains. In some embodiments, two or more first binding domains (e.g., antigen-binding fragments or cytokine moieties) are connected in tandem via optional linker(s). In some embodiments, the two or more first binding domains are on different antigen-binding polypeptides. In some embodiments, two or more second binding domains (e.g., antigen-binding fragments or cytokine moieties) are connected in tandem via optional linker(s). In some embodiments, the two or more second binding domains are on different antigen-binding polypeptides. In some embodiments, two or more third binding domains (e.g., antigen-binding fragments or cytokine moieties) are connected in tandem via optional linker(s). In some embodiments, the two or more third binding domains are on different antigen-binding polypeptides. In some embodiments, the two or more first binding domains are the same. In some embodiments, the two or more first binding domains are different. In some embodiments, the target molecule epitopes specifically recognized by the two or more first binding domains are the same. In some embodiments, the target molecule epitopes specifically recognized by the two or more first binding domains are different. In some embodiments, the two or more second binding domains are the same. In some embodiments, the two or more second binding domains are different. In some embodiments, the target molecule epitopes specifically recognized by the two or more second binding domains are the same. In some embodiments, the target molecule epitopes specifically recognized by the two or more second binding domains are different. In some embodiments, the two or more third binding domains are the same. In some embodiments, the two or more third binding domains are different. In some embodiments, the target molecule epitopes specifically recognized by the two or more third binding domains are the same. In some embodiments, the target molecule epitopes specifically recognized by the two or more third binding domains are different. For example, in some embodiments, the immunomodulatory molecule comprises from N′ to C′: Fab1-optional linker1-Fab2-optional linker2-(optional hinge or portion thereof-first binding domain (e.g., immunostimulatory cytokine moiety)-optional hinge or portion thereof)-Fc subunit. For example, CH1 or CL of Fab1 is linked to VH or VL of Fab2 via the optional linker 1. In some embodiments, the immunomodulatory molecule comprises from N′ to C′: scFv1 (or sdAb1)-optional linker1-scFv2 (or sdAb2)-optional linker 2-(optional hinge or portion thereof-first binding domain (e.g., immunostimulatory cytokine moiety)-optional hinge or portion thereof)-Fc subunit. In some embodiments, the immunomodulatory molecule comprises from N′ to C′: ligand1 (e.g., PD-L2)-optional linker1-ligand2 (e.g., PD-L2)-optional linker 2-(optional hinge or portion thereof-first binding domain (e.g., immunostimulatory cytokine moiety)-optional hinge or portion thereof)-Fc subunit. The first binding domain (e.g., immunostimulatory cytokine moiety) in parenthesis can be absent for the other pairing immunomodulatory molecule chain. For example, the immunomodulatory molecule can comprise a first polypeptide chain comprising from N′ to C′: scFv1 (or sdAb1)-optional linker1-scFv2 (or sdAb2)-optional linker2-first binding domain (e.g., immunostimulatory cytokine moiety)-hinge or portion thereof-Fc subunit1; and a second polypeptide chain from N′ to C′: scFv3 (or sdAb3)-optional linker3-scFv4 (or sdAb4)-optional linker4-hinge or portion thereof-Fc subunit2.

Binding affinity of a binding domain (e.g., scFv, Fab, VHH, ligand, or receptor) and its target molecule can be determined experimentally by any suitable antibody/antigen binding assays or other protein binding assays (e.g., ligand-receptor binding) known in the art, e.g., Western blots, ELISA, MSD electrochemiluminescence, bead based MIA, RIA, SPR, ECL, IRMA, EIA, Biacore assay, Octet analysis, peptide scans, FACS, etc. Also see “binding affinity” subsection below for exemplary methods. In some embodiments, the Kd of the binding between the antibody or antigen-binding fragment and its target molecule is about any of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M.

Amino acid sequence variants of an antigen-binding protein or binding domain (e.g., antigen-binding fragment) may be prepared by introducing appropriate modifications into the nucleic acid sequence encoding the antigen-binding protein or binding domain, or by peptide synthesis. Such modifications include, for example, deletions from, and/or insertions into and/or substitutions of residues within the amino acid sequences of the antigen-binding protein or binding domain. Any combination of deletion, insertion, and substitution can be made to arrive at the final construct, provided that the final construct possesses the desired characteristics, e.g., target molecule-binding.

In some embodiments, the antigen-binding protein (e.g., antibody or ligand/receptor-hinge-Fc fusion protein) or binding domain (e.g., scFv, Fab, VHH, ligand, or receptor) has one or more amino acid substitutions. Sites of interest for substitutional mutagenesis include the HVRs (or CDRs) and FRs of antibodies or antigen-binding fragments. Conservative substitutions are shown in Table B under the heading of “Preferred substitutions.” More substantial changes are provided in Table B under the heading of “exemplary substitutions,” and as further described below in reference to amino acid side chain classes. Amino acid substitutions may be introduced into a binding domain of interest and the products screened for a desired activity, e.g., retained/improved target molecule binding, decreased immunogenicity.

TABLE B
Amino acid substitutions

| Original | Exemplary | Preferred | Original | | Preferred |
|---|---|---|---|---|---|
| Residue | Substitutions | Substitutions | Residue | Exemplary Substitutions | Substitutions |
| Ala (A) | Val; Leu; Ile | Val | Leu (L) | Norleucine; Ile; Val; Met; | Ile |
| | | | | Ala; Phe | |
| Arg (R) | Lys; Gln; Asn | Lys | Lys (K) | Arg; Gln; Asn | Arg |
| Asn (N) | Gln; His; Asp, | Gln | Met (M) | Leu; Phe; Ile | Leu |
| | Lys; Arg | | | | |
| Asp (D) | Glu; Asn | Glu | Phe (F) | Trp; Leu; Val; Ile; Ala; Tyr | Tyr |
| Cys (C) | Ser; Ala | Ser | Pro (P) | Ala | Ala |
| Gln (Q) | Asn; Glu | Asn | Ser (S) | Thr | Thr |
| Glu (E) | Asp: Gln | Asp | Thr (T) | Val; Ser | Ser |
| Gly (G) | Ala | Ala | Trp (W) | Tyr; Phe | Tyr |
| His (H) | Asn; Gln; Lys; | Arg | Tyr (Y) | Trp; Phe; Thr; Ser | Phe |
| | Arg | | | | |
| Ile (I) | Leu; Val; Met; | Leu | Val (V) | Ile; Leu; Met; Phe; Ala; | Leu |
| | Ala; Phe; | | | Norleucine | |
| | Norleucine | | | | |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes for another class.

One type of substitutional variant involves substituting one or more HVR residues of a parent antibody or antigen-binding fragment thereof. Generally, the resulting variant(s) selected for further study will have modifications (e.g., improvements) in certain biological properties (e.g., increased affinity, reduced immunogenicity) relative to the parent antibody or antigen-binding fragment thereof, and/or will have substantially retained certain biological properties of the parent antibody or antigen-binding fragment thereof. An exemplary substitutional variant is an affinity-matured antibody, which may be conveniently generated, e.g., using phage display-based affinity maturation techniques such as those described herein. Briefly, one or more HVR residues are mutated and the variant antibodies displayed on phage and screened for a particular biological activity (e.g., binding affinity).

In some embodiments, substitutions, insertions, or deletions may occur within one or more HVRs so long as such alterations do not substantially reduce the ability of the antibody or antigen-binding fragment thereof to bind antigen. For example, conservative alterations (e.g., conservative substitutions as provided herein) that do not substantially reduce binding affinity may be made in HVRs. Such alterations may be outside of HVR “hotspots” or CDRs.

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

In some embodiments, the first binding domain is an immunostimulatory cytokine moiety or variant thereof, such as any of the cytokine moieties described herein (for example, any of SEQ ID NOs: 26-30, 41, 63-65, and 140). In some embodiments, the immunostimulatory cytokine moiety or variant thereof is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α G-CSF, M-CSF, SCF, and GM-CSF. In some embodiments, the first binding domain is an agonist antibody against T cell surface antigen, including but not limited to, CD3s, CD3δ, or CD3γ; or CD2, CD4, CD8, CD27, CD28, CD40, CD134, CD137, and CD278. In some embodiments, the first binding domain is an agonist antibody against NK cell surface antigen, including but not limited to, CD16a, CD56 (NCAM), NKp46, NKp44, CD244, CD226, TIGIT, CD96, LAG3, TIM3, PD-1, KLRG1, CD161, CD94/NKG2, KIR, NKG2D, and NKp30. In some embodiments, the first binding domain is an agonist antibody against any of CD27, CD28, CD137, OX40, GITR, and HVEM. In some embodiments, the first binding domain is an agonist ligand, such as CD80, CD86, or 4-1BB.

In some embodiments, the second binding domain is an agonist antibody against an inhibitory checkpoint molecule, such as PD-1, TIGIT, or CTLT-4. In some embodiments, the second binding domain is a ligand of an inhibitory checkpoint molecule, such as PD-L1, PD-L2, CD155, or variant thereof. In some embodiments, the second binding domain comprises the sequence of any of SEQ ID NOs: 106-110, 121-128, and 137.

In some embodiments, the third binding domain is an antibody (agonist, antagonist, or neutral) against T cell surface antigen, including but not limited to, CD3e, CD36, or CD37; or CD2, CD4, CD8, CD27, CD28, CD40, CD134, CD137, and CD278. In some embodiments, the third binding domain is an antibody (agonist, antagonist, or neutral) against NK cell surface antigen, including but not limited to, CD16a, CD56 (NCAM), NKp46, NKp44, CD244, CD226, TIGIT, CD96, LAG3, TIM3, PD-1, KLRG1, CD161, CD94/NKG2, KIR, NKG2D, and NKp30. In some embodiments, the third binding domain is an antibody (agonist, antagonist, or neutral) against T cell exhausted marker, including but not limited to PD-1, TIGIT, CTLA-4, LAG3, and TIM3. In some embodiments, the third binding domain is an antibody (agonist, antagonist, or neutral) against tumor antigen, including but not limited to Her2, Her3, CEA, Trop2, CLDN18.2. In some embodiments, the third binding domain is a ligand to an immune cell surface antigen (e.g., PD-1 or TIGIT as the antigen), such as PD-L1, PD-L2, CD155, or variant thereof. In some embodiments, the third binding domain comprises the sequence of any of SEQ ID NOs. 106-110, 121-128, and 137.

### Cytokines or Variants Thereof

Cytokines (also referred to as “cytokine molecule” or “cytokine protein” interchangeably) are secreted proteins that modulate the activity of cells of the immune system. Examples of cytokines include the interleukins, interferons, chemokines, lymphokines, tumor necrosis factors, colony-stimulating factors for immune cell precursors, and so on. In some embodiments, the cytokine is a wildtype cytokine. In some embodiments, the cytokine is a naturally existing cytokine species variant. In some embodiments, the cytokine is a naturally existing cytokine subtype. A “cytokine variant” herein refers to any cytokine molecule that is not naturally existing, such as a cytokine active fragment (e.g., a cytokine fragment that retains at least about 10% biological activity or cytokine receptor binding activity of a full-length cytokine), a mutant, or a derivative thereof. A “cytokine or variant thereof” is also interchangeably referred to herein as a “cytokine moiety,” which can be a cytokine molecule, or a species variant, subtype, active fragment, mutant, or derivative thereof.

As used herein, “heterodimeric cytokine” or “cytokine heterodimer” refers to a cytokine consisting of two distinct protein subunits. At present, IL-12 family (includes IL-12, IL-23, IL-27, and IL-35) is the only naturally occurring heterodimeric cytokine family that is known. However, artificial heterodimeric cytokines can be constructed. For example, IL-6 and a soluble fragment of IL-6R can be combined to form a heterodimeric cytokine, as can CNTF and CNTF-R alpha (Trinchieri (1994) Blood 84:4008). “Homodimeric cytokine” or “cytokine homodimer” herein refers to a cytokine consisting of two identical protein subunits, such as IFN-γ or IL-10. “Monomeric cytokine” or “cytokine monomer” refers to a cytokine that consists of one unit of cytokine molecule. In some embodiments, the cytokine or variant thereof is a monomeric cytokine or variant thereof. In some embodiments, the cytokine or variant thereof is a homodimeric cytokine or variant thereof. In some embodiments, the cytokine or variant thereof is a heterodimeric cytokine or variant thereof.

In some embodiments, the cytokine moiety is a full-length cytokine molecule. In some embodiments, the cytokine moiety is a functional fragment of the cytokine molecule that is capable of producing some (e.g., at least about 10%, 20%, 30%, 40%, 50%, 60%, 70%, 80%, 90%, or 95%) or full biological activity and/or cytokine receptor binding activity of a full-length cytokine molecule. In some embodiments, the cytokine moiety is a precursor cytokine molecule. In some embodiments, the cytokine moiety is a mature cytokine molecule (e.g., no signal peptide). In some embodiments, the cytokine moiety is a wild-type cytokine. In some embodiments, the cytokine moiety is a naturally existing cytokine species variant. In some embodiments, the cytokine moiety is a naturally existing cytokine subtype. In some embodiments, the cytokine moiety is a cytokine variant, such as a mutant cytokine capable of producing some (e.g., at least about 10%, 20%, 30%, 40%, 50%, 60%, 70%, 80%, 90%, or 95%) or full biological activity and/or cytokine receptor binding activity of a wild-type cytokine. In some embodiments, the cytokine variant is a modified cytokine, such as glycosylated cytokine. The cytokine or variant thereof described herein can be a cytokine isolated from a variety of sources, such as from human tissue types or from another source, or prepared by recombinant or synthetic methods. In some embodiments, the cytokine moiety is a recombinant cytokine. In some embodiments, the cytokine moiety described herein can be a cytokine derived from any organism, such as mammals, including, but are not limited to, livestock animals (e.g., cows, sheep, goats, cats, dogs, donkeys, and horses), primates (e.g., human and non-human primates such as monkeys or chimpanzees), rabbits, and rodents (e.g., mice, rats, gerbils, and hamsters). In some embodiments, the cytokine moiety is a human cytokine, such as recombinant human cytokine. In some embodiments, the cytokine moiety is a murine cytokine, such as recombinant murine cytokine. In some embodiments, the cytokine moiety is a mature human cytokine. In some embodiments, the cytokine moiety comprises a signal peptide at the N-terminus of the cytokine molecule, the signal peptide is either from a different molecule or from the same cytokine molecule.

Cytokine variants can be of truncated versions, post-translationally modified versions, hybrid variants, peptide mimetics, biologically active fragments, deletion variants, substitution variants, or addition variants that maintain at least some degree (e.g., at least about 100%) of the parental cytokine activity (cytokine receptor binding activity and/or biological activity). “Parental cytokine” or “parent cytokine” described herein refers to the cytokine reference sequence from which the cytokine variant is engineered, modified, or derived from.

When immunomodulatory molecule of the subject invention is described to contain two or more different cytokines (and optionally including additional protein moieties), it means that the immunomodulatory molecule contains two or more different cytokine molecules (rather than two or more different cytokine subunits). For example, a homodimeric cytokine (e.g. IFN-α, IFN-β, IFN-γ, IL-5, IL-8, or the like) is referred to herein a single cytokine molecule. For example, an immunomodulatory molecule comprising two IL-5 monomers/subunits (either on the same polypeptide chain as a single-chain fusion or on different polypeptide chains), is considered to contain only one cytokine molecule, i.e., IL-5. Similarly, a heterodimeric cytokine such as IL-12, although it contains different subunits, is a single cytokine. For example, an immunomodulatory molecule comprising a p35 subunit and a p40 subunit (either on the same polypeptide chain as a single-chain fusion or on different polypeptide chains), is considered to contain only one cytokine molecule, i.e., IL-12. Furthermore, a heterodimeric form of normally homodimeric cytokines, such as a MCP-1/MCP-2 heterodimer, or of two alleles of a normally homodimeric cytokine (e.g., Zhang, J. Biol. Chem. [1994] 269:15918-24) is a single cytokine. In some embodiments, the cytokine subunit (e.g., p35 of IL-12) on one polypeptide chain of an immunomodulatory molecule can dimerize with the pairing cytokine subunit (e.g., p40) either on the same polypeptide chain or on a different polypeptide chain within the same immunomodulatory molecule. In some embodiments, the cytokine subunit (e.g., p35 of IL-12) of an immunomodulatory molecule can dimerize with the pairing cytokine subunit (e.g., p40) of a nearby immunomodulatory molecule.

In some embodiments, the cytokine variant comprises a mutation or modification (e.g., post-translational modification) that results in selectivity against a first type of receptor (e.g., trimeric receptor, or higher affinity receptor) versus a second type of receptor of the corresponding cytokine molecule (e.g., dimeric receptor, or weaker affinity receptor), measured as a ratio of activation of cells expressing the first type of receptor relative to activation of cells expressing the second type of receptor. For example, in some embodiments, the cytokine variant is a mutant IL-2 (or post-translationally modified IL-2), which binds IL-2Rβγ with stronger affinity (e.g., at least about any of 2, 3, 4, 5, 6, 7, 8, 9, or 10-fold stronger affinity) compared to IL-2Rαβγ, or activates cells expressing IL-2Rβγ more than (e.g., at least about any of 2, 3, 4, 5, 6, 7, 8, 9, or 10-fold activation) those expressing IL-2Rαβγ; or vice versa. In some embodiments, depending on the disease types to be treated, the preferred mutations or alterations increase cytokine moiety's activation of immune effector cells (e.g., CD8+ T cells for treating cancer). For example, in some embodiments, the IL-2 variant has a mutation (or post-translationally modification) that reduces the IL-2 variant's activation of cells expressing IL-2Rβγ receptor relative to the IL-2 variant's activation of cells expressing IL-2Rαβγ receptor.

In some embodiments, the mutation or modification of the cytokine variant leads to a differential effect (e.g., such as reduced binding or cell activation), compared to an immunomodulatory molecule without mutation or modification to such cytokine moiety. In one aspect, the differential effect is measured by the proliferative response of cells or cell lines that depend on the cytokine (e.g., IL-2) for growth. This response to the immunomodulatory molecule is expressed as an EC50 value, which is obtained from plotting a dose response curve and determining the protein concentration that results in a half-maximal response. In some embodiments, the ratio of the EC50 values obtained for cells expressing the first receptor type (e.g., IL-2Rβγ receptor) to cells expressing the second receptor type (e.g., IL-2Rαβγ receptor) for an immunomodulatory molecule of the invention (e.g., IL-2 variant immunomodulatory molecule) relative to the ratio of EC50 values for a reference immunomodulatory molecule (e.g., IL-2 wildtype immunomodulatory molecule of the same configuration) gives a measure of the differential effect for the immunomodulatory molecule. In some embodiments, the EC50 value obtained for an immunomodulatory molecule of the invention (e.g., IL-2 variant immunomodulatory molecule) relative to the EC50 value for a reference immunomodulatory molecule (e.g., IL-2 wildtype immunomodulatory molecule of the same configuration) gives a measure of the differential effect for the immunomodulatory molecule.

In some embodiments, the cytokine variant includes a mutation in one or more amino acids of the parental cytokine molecule (e.g., mature wildtype cytokine). In one embodiment, the cytokine variant includes an amino acid substitution at one or more amino acid positions in the cytokine. In another embodiment, the cytokine variant includes deletions or insertions of amino acids at one or more amino acid positions in the cytokine. In some embodiments, the cytokine variant includes modifications of one or more amino acids in the cytokine.

In some embodiments, the cytokine or variant thereof is selected from the group consisting of IL-1, IL-2, IL-3, IL-4, IL-5, IL-6, IL-7, IL-8, IL-9, IL-10, IL-12, IL-15, IL-17, IL-18, IL-21, IL-22, IL-23, IL-27, IL-35, IFN-α, IFN-β, IFN-γ, TNF-α, TGF-β, VEGF, erythropoietin, thrombopoietin, G-CSF, M-CSF, SCF, and GM-CSF, or natural variants or subtypes thereof. In some embodiments, the cytokine or variant thereof is an anti-inflammatory or immunosuppressive cytokine or variant thereof, such as IL-1Ra, IL-4, IL-5, IL-6, IL-10, IL-11, IL-13, IL-27, IL-33, IL-35, IL-37, IL-39, IFN-α, LIF, or TGF-β. In some embodiments, the cytokine or variant thereof is a pro-inflammatory or immunostimulatory cytokine or variant thereof, such as IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, or GM-CSF, or variant or subtype thereof. In some embodiments, the cytokine or variant thereof is selected from the group consisting of IL-2, IL-10, IL-12, IL-23, IFN-α (e.g., IFN-α2 or IFN-α2b), IFN-β, and IFN-γ. In some embodiments, the immunostimulatory cytokine is IL-12, and the cytokine subunits are p35 and p40. In some embodiments, the immunostimulatory cytokine is IL-23, and the cytokine subunits are p40 and p19. In some embodiments, the cytokine is LL-27, and the cytokine subunits are Epstein-Barr virus-induced gene 3 (EB13) and IL-27p28. In some embodiments, the immunosuppressive cytokine is IL-35, and the cytokine subunits are IL-12α (p35) and IL-27β. In some embodiments, the cytokine variant is a single chain fusion of two or more subunits from different cytokines.

### IL-2

In some embodiments, the immunostimulatory cytokine or variant thereof is IL-2 or variant thereof. Interleukin-2 (IL-2), also known as T cell growth factor (TCGF), is a 15.5 kDa monomeric protein that plays a key role in lymphocyte generation, survival, and homeostasis. It is involved in body's natural response to microbial infection and discriminating between “self” and “non-self.” IL-2 is an interleukin, it belongs to a cytokine family that includes IL-4, IL-7, IL-9, IL-15, and IL-21. IL-2 mediates its effects by binding to IL-2 receptors (IL-2R) expressed on lymphocytes. Activated CD4⁺ T cells and activated CD8⁺ T cells are the major sources of IL-2. Its ability to expand lymphocyte populations and increase effector functions of these cells makes IL-2 an attractive therapy against cancer. IL-2 has been suggested for treating acute myeloid leukemia (AML), non-Hodgkin's lymphoma (NHL), cutaneous T-cell lymphoma (CTCL), breast cancer, and bladder cancer.

IL-2 receptor (IL-2R) is a complex consisting of three chains, α (CD25, p55), β (CD122, p75), and γ (CD132, p65). The γ chain is shared by all IL-2 cytokine family members. IL-2 binding to either intermediate-affinity dimeric CD122/CD132 IL-2R (IL-2Rβγ, Kd˜10⁻⁹ M) or high-affinity trimeric CD25/CD122/CD132 IL-2R (IL-2Rαβγ, Kd˜10⁻¹¹ M) can lead to signal transduction, while binding to CD25 alone cannot. The β chain is complexed with Janus kinase 1 (JAK1). The γ chain is complexed with JAK3. Upon IL-2 binding to IL-2R, JAK1 and JAK3 are activated and capable of adding phosphate groups to molecules, thus initiating three intracellular signaling pathways: the MAP kinase pathway, the Phosphoinositide 3-kinase (PI3K) pathway, and the JAK-STAT pathway. Dimeric IL-2Rβγ is expressed by memory CD8⁺ T cells, NK cells, and B cells, whereas high levels of trimeric IL-2Rαβγ is expressed by regulatory T cells (Tregs) and activated T cells.

Aldesleukin (Proleukin®), recombinant human IL-2, was the first cancer immunotherapy, and one of the first recombinant proteins, approved by the FDA in 1992. Currently, Aldesleukin is used for the treatment of metastatic renal cell carcinoma (mRCC) and metastatic melanoma (mM) by IV infusion. Due to the requirement of frequent intravenous infusion over multiple doses, administration of Aldesleukin occurs within a clinical setting. Aldesleukin has demonstrated complete cancer regression in about 10% of patients treated for metastatic melanoma and renal cancer (Kapper et al., Cancer, 2008; Rosenberg, Sci Transl Med., 2012; Smith et al., Clin Cancer Res., 2008). Approximately 70% of patients with complete responses have been cured, maintaining complete regression for more than 25 years after initial treatment (Atkins et al., J Clin Oncol., 1999; Klapper et al., Cancer, 2008; Rosenberg, Sci Transl Med., 2012; Rosenberg et al., Ann Surg., 1998; Smith et al., Clin Cancer Res., 2008). However, high doses of IL-2 can induce vascular leak syndrome (VLS), tumor tolerance caused by activation-induced cell death (AICD), and immunosuppression caused by the activation of Tregs. An additional concern of systemic IL-2 treatment is related to severe side effects upon intravenous administration, which include severe cardiovascular, pulmonary edema, hepatic, gastrointestinal (GI), neurological, and hematological events (Proleukin (aldesleukin) Summary of Product Characteristics [SmPC]: http://www.medicines.org.uk/emc/medicine/19322/SPC). The severe side effects often restrict optimal IIL-2 dosing, which limits the number of patients who successfully respond to therapy. For more prevalent application in the future, toxicity and short half-life concerns of IL-2 need to be addressed.

Native human IL-2 precursor polypeptide consists of 153 amino acid residues (amino acids 1-20 are signal peptide), while the mature polypeptide consists of 133 amino acid residues (SEQ ID NO: 25). In some embodiments, the IL-2 moiety is a human mature IL-2. In some embodiments, the IL-2 moiety is a polypeptide substantially homologous to amino acid sequence of a wild-type human IL-2 (SEQ ID NO: 25), e.g., having at least about 85% (such as at least about any of 90°/%, 91%, 92%, 93%, 94%, 95%, 96%, 97%, 98%, 99%, or 100%) amino acid sequence identity to a wild-type human IL-2 (SEQ ID NO: 25). In some embodiments, the IL-2 moiety is not glycosylated. In some embodiments, the IL-2 moiety is glycosylated.

In some embodiments, the IL-2 moiety is (or consists essentially of) Aldesleukin (e.g., Proleukin®; see, e.g., https://www.drugbank.ca/drugs/DB00041). Aldesleukin (desalanyl-1, serine-125 human interleukin-2) is an antineoplastic (anticancer) biologic response modifier approved by the FDA. It has a molecular weight of approximately 15.3 kDa, and synonym recombinant interleukin-2 human, Interleukin-2 aldesleukin, 125-L-serine-2-133-interleukin 2 (human reduced), or Interleukin-2(2-133), 125-ser. Aldesleukin is a recombinant IL-2, it differs from native IL-2 in the following ways: a) Aldesleukin is not glycosylated because it is produced from E. coli; b) Aldesleukin has no N-terminal alanine (A); c) Aldesleukin has a cysteine to serine substitution at position 125 (C125S); and d) the aggregation state of Aldesleukin is likely different from that of native IL-2. Thus in some embodiments, the IL-2 variant comprises a cysteine to serine substitution at position 125 (C125S) from the human IL-2 mature form.

K. Sauvé et al. (Proc Natl Acad Sci USA. 1991: 88(11):4636-4640) found that amino acid residues K35, R38, F42, and K43 of wildtype IL-2 were found to be crucial for IL-2 receptor binding (IL-2Rα, low affinity form), and R38A and F24A mutations retained substantial IL-2 biological activity. R. Vazquez-Lombardi et al. (Nat Commun. 2017; 8:15373) discovered that R38D, K43E, and E61R mutations in IL-2 drove strong expansion of CD25⁻ cytotoxic subsets with minimal expansion of Tregs compared to wildtype IL-2. P65L mutation in IL-2 was found to have reduced systemic toxicity and greater antitumor efficacy compared to wildtype IL-2 (Chen et al., Cell Death Dis. 2018; 9(10):989).

In some embodiments, the IL-2 variant comprises one or more mutations at a position selected from the group consisting of L18, Q22, F24, K35, R38, F42, K43, E61, P65, Q126, and S130 relative to a wildtype IL-2 (SEQ ID NO: 25). In some embodiments, the IL-2 variant comprises one or more mutations selected from the group consisting of L18R, Q22E, F24A, R38D, K43E, E61R, P65L, Q126T, and S130R relative to a wildtype IL-2 (SEQ ID NO: 25). In some embodiments, the IL-2 variant comprises an R38D/K43E/E61R mutation relative to a wildtype IL-2 (SEQ ID NO: 25). In some embodiments, the IL-2 variant comprises the sequence of SEQ ID NO: 26. In some embodiments, the IL-2 variant comprises an L18R/Q22E/R38D/K43E/E61R mutation relative to SEQ ID NO: 25. In some embodiments, the IL-2 variant comprises the sequence of SEQ ID NO: 27. In some embodiments, the IL-2 variant comprises an R38D/K43E/E61R/Q126T mutation relative to SEQ ID NO: 25. In some embodiments, the IL-2 variant comprises the sequence of SEQ ID NO: 28. In some embodiments, the IL-2 variant comprises an L18R/Q22E/R38D/K43E/E61R/Q126T mutation relative to SEQ ID NO: 25. In some embodiments, the TL-2 variant comprises the sequence of SEQ ID NO: 29. In some embodiments, the IL-2 variant comprises an L18R/Q22E/R38D/K43E/E61R/Q126T/S130R mutation relative to SEQ ID NO: 25. In some embodiments, the IL-2 variant comprises the sequence of SEQ ID NO: 30.

### IFN-α

In some embodiments, the immunostimulatory cytokine or variant thereof is IFN-α or variant thereof, such as IFN-α2 or variant thereof, or IFN-α2b or variant thereof. Human type 1 interferons (IFNs) are a large group of IFNs that help regulate the activity of the immune system. They bind to a specific cell surface receptor complex known as the IFN-α receptor (IFNAR) consisting of IFNAR1 and IFNAR2 chains. Mammalian type I IFNs contain IFN-α, IFN-β, IFN-κ, IFN-δ, IFN-ε, IFN-τ, IFN-ω, and IFN-ζ (a.k.a. limitin).

IFN-α proteins are mainly produced by plasmacytoid dendritic cells (pDCs), and mainly involved in innate immunity against viral infection. IFN-α proteins are 19-26 kDa monomeric proteins that have been extensively used for the treatment of cancer and viral diseases, such as Hepatitis B and C. There are 13 genes responsible for synthesis of 13 IFN-α subtypes: IFNA1, IFNA2, IFNA4, IFNA5, IFNA6, IFNA7, IFNA8, IFNA10, IFNA13, IFNA14, IFNA16, IFNA17, IFNA21.

Human IFN-α2a, IFN-α2b, and IFN-α2c represent allelic variants of the same gene. IFN-α2a and IFN-α2b have a lysine and an arginine at position 23 of the mature protein, respectively. Human IFN-α2a and IFN-α2b are the only IFN-α subtypes with an O-glycosylation site (on Thr106). Interferon alfa-2a (IFN-α2a; marketed by Hoffmann-La Roche as Roferon-A®) and interferon alfa-2b (IFN-α2b, recombinant form of IFN-α2; marketed by Schering-Plough as Intron-A®) have been approved for the treatment of hairy cell leukemia, melanoma, follicular lymphoma, renal cell carcinoma, AIDS-related Kaposi's sarcoma, and chronic myelogenous leukemia (M. Ferrantini et al., Biochimie. June-July 2007; 89(6-7):884-893). Recent studies have underscored new immunomodulatory effects of IFN-α, including activities on T cells and dendritic cells, which may lead to generation of a durable antitumor response. However, the use of IFN-α in clinical oncology is still generally based on exploiting the anti-proliferative and anti-angiogenic activities of these cytokines. Full exploit of the role of IFN-α as a regulator of immune response and tumor immunity would require novel approaches in the use of these cytokines.

hIFN-α2b is a glycoprotein consisting of 166 amino acids with O-glycosylated threonine at position 106. Each rhIFN-2b consists of five a helices (called helix A to E) connected by a loop AB, BC, CD, and DE. Residues that are important in receptor binding are the AB loop (Arg22, Leu26, Phe27, Leu30, Lys31, Arg33, and His34), helix B (Ser68), helix C (Thr79, Lys83, Tyr85, and Tyr89), D helix (Arg120, lys121, Gln124, Lys131, and Glu132), and helix E (Arg144 and Glu146). Amino acid residues that are important in the biological activity are Leu30, Lys31, Arg33, His34, Phe36, Arg120, Lys121, Gln124, Tyr122, Tyr129, Lys131, Glu132, Arg144, and Glu146 (Ratih Asmana Ningrum, Scientifica (Cairo). 2014; 2014:970315).

In some embodiments, the IFN-α moiety is IFN-α2. In some embodiments, the IFN-α moiety is IFN-α2a. In some embodiments, the IFN-α moiety is IFN-α2b. In some embodiments, the IFN-α moiety is IFN-α2c. In some embodiments, the IFN-α moiety is a mature IFN-α. Native human IFN-α2b precursor polypeptide consists of 188 amino acid residues (amino acids 1-23 are signal peptide), while the mature polypeptide consists of 165 amino acid residues (SEQ ID NO: 31). In some embodiments, the IFN-α moiety is a polypeptide substantially homologous to a wild-type IFN-α (SEQ ID NO: 31), e.g., having at least about 85% (such as at least about any of 90%, 91%, 92%, 93%, 94%, 95%, 96%, 97%, 98%, 99%, or 100%) amino acid sequence identity to a wild-type IFN-α (SEQ ID NO: 31). In some embodiments, the IFN-α moiety is not glycosylated. In some embodiments, the IFN-α moiety is glycosylated.

In some embodiments, the IFN-α variant (e.g., IFN-α2b variant) comprises one or more mutations at a position selected from the group consisting of R22, L26, F27, L30, K31, D32, R33, H34, D35, F36, S68, T79, K83, Y85, Y89, R120, K121, Y122, Q124, Y129, K131, E132, R144, and E146 relative to an IFN-α (e.g., IFN-α2b; SEQ ID NO: 31). In some embodiments, the IFN-α variant (e.g., IFN-α2b variant) comprises one or more mutations selected from the group consisting of L30A, K31A, D32A, R33A, H34A, and D35A relative to an IFN-α (e.g., IFN-α2b; SEQ ID NO: 31). In some embodiments, the IFN-α variant (e.g., IFN-α2b variant) comprises an L30A mutation relative to an IFN-α (e.g., IFN-α2b; SEQ ID NO: 31). In some embodiments, the IFN-α variant comprises an amino acid sequence of SEQ ID NO: 32. In some embodiments, the IFN-α variant (e.g., IFN-α2b variant) comprises the sequence of any of SEQ ID NOs: 32-37.

### IFN-β

Two types of IFN-β have been described, IFN-β1 and IFN-β3. In some embodiments, the immunostimulatory cytokine or variant thereof is IFN-β or variant thereof, such as IFN-β1, IFN-β3, or variant thereof. In some embodiments, the immunostimulatory cytokine or variant thereof is IFN-β1a or variant thereof. In some embodiments, the IFN-γ moiety is a mature IFN-β. In some embodiments, the IFN-β moiety is a wildtype (e.g., wildtype human) IFN-β. In some embodiments, the IFN-β moiety is a mutant (e.g., mutant human) IFN-β. In some embodiments, the IFN-β moiety is not glycosylated. In some embodiments, the IFN-β moiety is glycosylated.

### IFN-γ

In some embodiments, the immunostimulatory cytokine or variant thereof is IFN-γ or variant thereof. Interferon gamma (IFNγ) is a disulfide-linked dimerized soluble cytokine that is the only member of the type II class of interferons. IFN-γ is a homodimer of −25 kDa with a tertiary fold built around an unusual pattern of interdigitating a helices. It is produced predominantly by T cells and NK cells in response to a variety of inflammatory or immune stimuli. IFN-γ can serve both as an immune system activator and suppressor. Studies showed that cancer immunotherapy (checkpoint inhibitors) acts partially through an increase of IFN-γ expression, leading to the elimination of cancer cells. Resistance to immunotherapy is attributed to defects in IFN-γ signaling. However, IFN-γ can also contribute to cancer evasion by promoting tumorigenesis and angiogenesis, eliciting expression of tolerant molecules such as PD-L1, and inducing homeostasis program. Due to its opposite and competing effects on the immune system, IFN-γ has not been approved by FDA to treat cancer patients except in the case of malignant osteoporosis (L. Ni and J. Lu, Cancer Med. 2018; 7(9):4509-4516).

Monomeric native human IFN-γ (hIFN-γ) pre-pro-polypeptide consists of 166 amino acid residues (amino acids 1-23 are signal peptide); the monomeric mature polypeptide consists of 138 amino acid residues (SEQ ID NO: 38), corresponding to amino acids 24-161 of the pre-pro-polypeptide; amino acids 162-166 are propeptide sequence of the pre-pro-polypeptide. In some embodiments, the monomeric IFN-γ moiety is a monomeric mature IFN-γ. In some embodiments, the monomeric IFN-γ moiety is a polypeptide substantially homologous to a wild-type IFN-γ (SEQ ID NO: 38), e.g., having at least about 85% (such as at least about any of 90%, 91%, 92%, 93%, 94%, 95%, 96%, 97%, 98%, 99%, or 100%) amino acid sequence identity to a wild-type IFN-γ (SEQ ID NO: 38). In some embodiments, the IFN-γ moiety (or subunit) is not glycosylated. In some embodiments, the IFN-γ moiety (or subunit) is glycosylated. In some embodiments, the IFN-γ moiety comprises two identical IFN-γ monomers/subunits. In some embodiments, the IFN-γ moiety comprises two different IFN-γ monomers/subunits. For example, in some embodiments, the IFN-γ moiety comprises one wildtype IFN-γ monomer and one IFN-γ variant monomer. In some embodiments, the IFN-γ moiety comprises two IFN-γ monomers (e.g., two IFN-γ variant or wildtype monomers) linked together, such as via a peptide linker (e.g., any of SEQ ID NOs: 227-229, 245, and 246) or a chemical linker.

IFN-γ amino acid residues S20, A23, H111, and Q115 are important for receptor binding; amino acid residues V5, S20, A23, G26, and H111 are important for IFN-γ biological activity (M. Randal and A. A. Kossiakoff, Structure. 2001; 9(2):155-63). Lander et al. (J Mol Biol. 2000; 299(1):169-79) developed a biologically active single chain variant of hIFN-γ (IFN-γSC1), by linking two monomeric IFN-γ with a 7-amino acid residue linker and changing His111 in the first IFN-γ monomer to an aspartic acid residue. Due to the H1 ID mutation, IFN-γSC1 can only bind one IFN-γRα but can fully retain its biological activity in cell proliferation, MHC class 1 induction, and anti-viral assays.

In some embodiments, the monomeric IFN-γ comprises the sequence of SEQ ID NO: 38. In some embodiments, the IFN-γ variant comprises one or more mutations within one or both IFN-γ subunits at a position selected from the group consisting of V5, S20, D21, V22, A23, D24, N25, G26, H111, and Q115 relative to a wildtype IFN-γ subunit (SEQ ID NO: 38). In some embodiments, the IFN-γ variant comprises one or more mutations within one or both IFN-γ subunits selected from the group consisting of S20A, D21A, D21K, V22A, A23S, A23E, A23Q, A23V, D24A, D24E, N25A, N25K, and H111ID relative to a wildtype IFN-γ subunit (SEQ ID NO: 38). In some embodiments, the IFN-γ variant comprises one or more mutations within one or both IFN-γ subunits selected from the group consisting of S20A/D21A, D21K, V22A/A23S, D24A/N25A, A23E/D24E/N25K, A23Q, and A23V relative to a wildtype IFN-γ subunit (SEQ ID NO: 38). In some embodiments, one or both subunits of the IFN-γ variant comprises the sequence of any of SEQ ID NOs: 39-45. In some embodiments, the IFN-γ variant comprises an A23V mutation within one or both IFN-γ subunits relative to a wildtype IFN-γ subunit (SEQ ID NO: 38). In some embodiments, the one or both subunits of the IFN-γ variant comprises the sequence of SEQ ID NO: 41. In some embodiments, the two subunits of the IFN-γ or variant thereof are connected by a linker (e.g., any of SEQ ID NOs: 227-229, 245, and 246). In some embodiments, the IFN-γ variant comprises the sequence of SEQ ID NO: 47 or 252. In some embodiments, both subunits of the IFN-γ comprises the sequence of SEQ ID NO: 38. In some embodiments, the IFN-γ moiety is a recombinant “wildtype” IFN-γ comprising two wildtype IFN-γ subunits connected by a linker (e.g., any of SEQ ID NOs: 227-229, 245, and 246), such as comprising the sequence of SEQ ID NO: 46 or 251.

### IL-10

In some embodiments, the immunosuppressive cytokine or variant thereof is IL-10 or variant thereof. Interleukin 10 (IL-10) is an α-helical cytokine that is expressed as a non-covalently linked homodimer of −37 kDa, also known as human cytokine synthesis inhibitory factor (CSIF). It plays a key role in the induction and maintenance of tolerance. IL-10 signals through a JAK-STAT complex. The IL-10 receptor (IL-10R) has two subunits, an a subunit that is primarily expressed on immune cells, particularly monocytes and macrophages with the highest expression, and an ubiquitously expressed β subunit. IL-10 is mainly produced by monocytes and, to a lesser extent, lymphocytes, including type-II T helper cells (TH2), mast cells, CD4⁺CD25⁺Foxp3-regulatory T cells, and subsets of activated T cells and B cells. Dendritic cells and NK cells can also produce IL-10. IL-10 suppresses the secretion of pro-inflammatory cytokines like TNFα, IL-1, IL-6, IL-12 as well as Th1 cytokines such as IL-2 and IFN-γ and controls differentiation and proliferation of macrophages, B-cells and T-cells (Glocker, E. O. et al., Ann. N.Y. Acad. Sci. 1246, 102-107 (2011); Moore, K. W. et al., Annu. Rev. Immunol. 19, 683-765 (2001); R. de Waal Malefyt et al., J. Erp. Med. 174, 915-924 (1991); Williams, L. M. et al., Immunology 113, 281-292 (2004)). Moreover, it is a potent inhibitor of antigen presentation, inhibiting MHC II expression as well as upregulation of co-stimulatory molecules CD80 and CD86 (Mosser, D. M. & Zhang, X. Immunological Reviews 226, 205-218 (2008)). If IL-10 is not present or not functional, inflammation cannot be controlled. This makes IL-10 an attractive therapeutic candidate for autoimmune diseases. However, clinical trials using IL-10 and the development of a recombinant IL-10 (ilodecakin, TENOVIL®, Schering-Plough Research Institute, Kenilworth, N.J.) have been discontinued due to lack of efficacy. Recent studies have shed light on IL-10's potential role in tumor treatment (Fujii et al., (October 2001). “Interleukin-10 promotes the maintenance of antitumor CD8(+) T-cell effector function in situ”. Blood. 98(7):2143-51).

Monomeric native human IL-10 precursor polypeptide consists of 178 amino acid residues (amino acids 1-18 are signal peptide), while the monomeric mature IL-10 polypeptide consists of 160 amino acid residues (SEQ ID NO: 52). In some embodiments, the monomeric IL-10 moiety is a monomeric mature IL-10. In some embodiments, the monomeric IL-10 moiety is a polypeptide substantially homologous to a wild-type IL-10 (SEQ ID NO: 52), e.g., having at least about 85% (such as at least about any of 90%, 91%, 92%, 93%, 94%, 95%, 96%, 97%, 98%, 99%, or 100%) amino acid sequence identity to a wild-type IL-10 (SEQ ID NO: 52). In some embodiments, the IL-10 moiety (or subunit) is not glycosylated. In some embodiments, the IL-10 moiety (or subunit) is glycosylated. In some embodiments, the IL-10 moiety comprises two identical IL-10 monomers/subunits. In some embodiments, the IL-10 moiety comprises two different IL-10 monomers/subunits. For example, in some embodiments, the IL-10 moiety comprises one wildtype IL-10 monomer and one IL-10 variant monomer. In some embodiments, the IL-10 moiety comprises two IL-10 monomers (e.g., two IL-10 variant or wildtype monomers) linked together, such as via a peptide linker (e.g., any of SEQ ID NOs: 227-229, 245, and 246) or a chemical linker, see e.g., a biologically active single chain IL-10 in US20130316404, the content of which is incorporated herein by reference in its entirety.

IL-10 amino acid residues N21, M22, R24, R32, H90, S31, and S93 are important in IL-10 receptor binding; residue R24 is crucial for IL-10 biological activity (Yoon et al., J Biol Chem. 2006; 281(46):35088-35096; E. S. Acuner-Ozbabacan et al. BMC Genomics. 2014; 15 Suppl 4(Suppl 4):S2).

In some embodiments, the monomeric IL-10 comprises the sequence of SEQ ID NO: 52. In some embodiments, the IL-10 variant comprises one or more mutations within one or both IL-10 subunits at a position selected from the group consisting of N21, M22, R24, D25, L26, R27, D28, A29, F30, S31, R32, H90, and S93 relative to a wildtype IL-10 subunit (SEQ ID NO: 52). In some embodiments, the IL-10 variant comprises one or more mutations within one or both IL-10 subunits selected from the group consisting of R24A, D25A, L26A, R27A, D28A, A29S, F30A, S31A, and R32A relative to a wildtype TL-10 subunit (SEQ ID NO: 52). In some embodiments, the IL-10 variant comprises one or more mutations within one or both IL-10 subunits selected from the group consisting of R24A, D25A/L26A, R27A, D28A/A29S, F30A/S31A, and R32A relative to a wildtype IL-10 subunit (SEQ ID NO: 52). In some embodiments, the one or both subunits of the IL-10 variant comprises the sequence of any of SEQ ID NOs: 53-58. In some embodiments, the IL-10 variant comprises an R27A mutation within one or both IL-10 subunits relative to a wildtype IL-10 subunit (SEQ ID NO: 52). In some embodiments, the one or both subunits of the IL-10 variant comprises the sequence of SEQ ID NO: 55. In some embodiments, the IL-10 variant comprises the sequence of SEQ ID NO: 60. In some embodiments, both subunits of IL-10 comprises the sequence of SEQ ID NO: 52. In some embodiments, the two subunits of the IL-10 or variant thereof are connected by a linker. In some embodiments, the IL-10 moiety is a recombinant “wildtype” IL-10 comprising two wildtype IL-10 monomers connected by a linker (e.g., any of SEQ ID NOs: 227-229, 245, and 246), such as comprising the sequence of SEQ ID NO: 59.

### IL-12

In some embodiments, the immunostimulatory cytokine or variant thereof is IL-12 or variant thereof. IL-12 is a 70 kDa heterodimeric protein consisting of two covalently (disulfide bond) linked p35 (IL-12A) and p40 (IL-12B) subunits. P40 subunit is shared between IL-12 and IL-23. The active heterodimer (referred to as “p70”), and a homodimer of p40 are formed following protein synthesis. IL-12 is an interleukin belonging to the IL-12 family, which is the only family comprising heterodimeric cytokines, including IL-12, IL-23, IL-27, and IL-35. IL-12 is produced by dendritic cells, macrophages, neutrophils, and human B-lymphoblastoid cells (NC-37) in response to antigenic stimulation. IL-12 functions by binding to the IL-12 receptor (IL-12R), which is a heterodimeric receptor formed by IL-12Rβ1 and IL-12Rβ2, and in turn leading to JAK-STAT pathway activation. IL-12 promotes the development of Th1 responses and greatly induces IFNγ production by T and NK cells. IL-12's ability to activate both innate (NK cells) and adaptive (cytotoxic T lymphocytes) immunities has made it a promising candidate for cancer immunotherapy. Despite positive results from animal trials, IL-12 has only showed modest anti-tumor responses in clinical trials and was often accompanied by significant issues with toxicity (Lasek et al., Cancer Immunol Immunother., 2014). Treatment with IL-12 was associated with systemic flu-like symptoms (fever, chills, fatigue, erythromelalgia, or headache) and toxic effects on the bone marrow and liver. Dosing studies showed that patients could only tolerate doses under 1 μg/kg, far below the therapeutic dose. The result is that clinical trials with IL-12—used either as monotherapy or combined with other agents—failed to demonstrate potent sustained therapeutic efficacy ((Lasek et al., Cancer Immunol Immunother., 2014).

Native human p35 (IL-12A) precursor polypeptide consists of 219 amino acid residues (amino acids 1-22 are signal peptide), while the mature polypeptide consists of 197 amino acid residues (SEQ ID NO: 61). Native human p40 (IL-12B) precursor polypeptide consists of 328 amino acid residues (amino acids 1-22 are signal peptide), while the mature polypeptide consists of 306 amino acid residues (SEQ ID NO: 62). In some embodiments, the IL-12 moiety (or IL-12 subunit) is a mature IL-12 (or mature subunit). In some embodiments, the IL-12A (p35) subunit or variant thereof is a polypeptide substantially homologous to amino acid sequence of a wild-type IL-12A (p35) (SEQ ID NO: 61), e.g., having at least about 85% (such as at least about any of 90°/%, 91%, 92%, 93%, 94%, 95%, 96%, 97%, 98%, 99%, or 100%) amino acid sequence identity to a wild-type IL-12A (p35) (SEQ ID NO: 61). In some embodiments, the IL-12B (p40) subunit or variant thereof is a polypeptide substantially homologous to amino acid sequence of a wild-type IL-12B (p40) subunit (SEQ ID NO: 62), e.g., having at least about 85% (such as at least about any of 90%, 91%, 92%, 93%, 94%, 95%, 96%, 97%, 98%, 99%, or 100%) amino acid sequence identity to a wild-type IL-12B (p40) subunit (SEQ ID NO: 62). In some embodiments, the IL-12 (or subunit) or variant thereof is not glycosylated. In some embodiments, the IL-12 (or subunit) or variant thereof is glycosylated. In some embodiments, the IL-12 variant comprises one wildtype subunit (e.g., wt p35) and one mutant subunit (e.g., variant p40). In some embodiments, the IL-12 variant comprises two variant subunits (p35 variant and p40 variant). In some embodiments, the IL-12 variant comprises two wildtype subunits (e.g., wt p35 and p40) that are linked together via a synthetic peptide linker (e.g., any of SEQ ID NOs: 227-229, 245, and 246) or a chemical linker.

Within the p40 subunit, amino acid residues that are important for IL-2 receptor binding are C177, E45, E59, and D62(Luo et al. J Mol Biol. 2010; 402(5):797-812). Studies suggested that an accessible N terminus of the p40 subunit is important for IL-12 bioactivity. Lieschke et al. constructed a single chain IL-12 (scIL-12) and noted that the order of the subunits affected IL-12 biologic activity: when the p35 subunit was at the N-terminus of p40 subunit, IL-12 activity greatly decreased; when p40 subunit was at the N-terminus of the p35 subunit, scIL-12 had biological activity comparable to rIL-12 (Lieschke et al. Nat Biotechnol. 1997; 15(1):35-40).

In some embodiments, the IL-12 moiety comprises a wildtype p35 subunit (SEQ ID NO: 61). In some embodiments, the IL-12 moiety comprises a variant p35 subunit. In some embodiments, the IL-12 moiety comprises a wildtype p40 subunit (SEQ ID NO: 62). In some embodiments, the IL-12 moiety comprises a variant p40 subunit. In some embodiments, the IL-12 moiety comprises a wildtype or variant p35 subunit and a wildtype or variant p40 subunit connected by a peptide linker (e.g., any of SEQ ID NOs: 227-229, 245, and 246). In some embodiments, the IL-12 moiety comprises from N-terminus to C-terminus: wildtype or variant p40 subunit-linker (e.g., any of SEQ ID NOs: 227-229, 245, and 246)-wildtype or variant p35 subunit. In some embodiments, the IL-12 moiety comprises from N-terminus to C-terminus: wildtype or variant p35 subunit-linker (e.g., any of SEQ ID NOs: 227-229, 245, and 246)-wildtype or variant p40 subunit. In some embodiments, the IL-12 variant comprises one or more mutations within the p40 subunit at a position selected from the group consisting of E45, Q56, V57, K58, E59, F60, G61, D62, A63, G64, Q65, and C177 relative to a wildtype p40 subunit (SEQ ID NO: 62). In some embodiments, the IL-12 variant comprises one or more mutations within the p40 subunit selected from the group consisting of Q56A, V57A, K58A, E59A, F60A, F60D, G61A, D62A, A63S, G64A, and Q65A relative to a wildtype p40 subunit (SEQ ID NO: 62). In some embodiments, the p40 subunit of the IL-12 variant comprises the sequence of any of SEQ ID NOs: 63-66 and 140. In some embodiments, the IL-12 variant comprises an E59A/F60A mutation within the p40 subunit relative to a wildtype p40 subunit (SEQ ID NO: 62). In some embodiments, the p40 subunit of the IL-12 variant comprises the sequence of SEQ ID NO: 63. In some embodiments, the IL-12 variant comprises an F60A mutation within the p40 subunit relative to a wildtype p40 subunit (SEQ ID NO: 62). In some embodiments, the p40 subunit of the IL-12 variant comprises the sequence of SEQ ID NO: 65. In some embodiments, the IL-12 variant comprises an F60D mutation within the p40 subunit relative to a wildtype p40 subunit (SEQ ID NO: 62). In some embodiments, the p40 subunit of the IL-12 variant comprises the sequence of SEQ ID NO: 140. In some embodiments, the p40 subunit and the p35 subunit of the IL-12 or variant thereof are connected by a linker (e.g., any of SEQ ID NOs: 227-229, 245, and 246). In some embodiments, the IL-12 variant comprises the sequence of any one of SEQ ID NOs: 68-71 and 254. In some embodiments, the IL-12 moiety is a recombinant “wildtype” IL-12 comprising a wildtype p35 subunit and a wildtype p40 subunit connected by a linker (e.g., any of SEQ ID NOs: 227-229, 245, and 246), such as comprising the sequence of SEQ ID NO: 67 or 253.

In some embodiments, the IL-12 moiety is derived from mouse IL-12. The mouse p35 subunit and/or the p40 subunit can be wildtype or variant. In some embodiments, the mouse IL-12 variant comprises one or two mutations within the p40 subunit at one or both positions of E59 and F60 relative to a mouse wildtype p40 subunit. In some embodiments, the p40 subunit and the p35 subunit of the mouse IL-12 or variant thereof are connected by a linker (e.g., any of SEQ ID NOs: 227-229, 245, and 246). In some embodiments, the mouse IL-12 variant comprises the sequence of SEQ ID NO: 72.

### IL-23

In some embodiments, the immunostimulatory cytokine or variant thereof is IL-23 or variant thereof. Interleukin-23 (IL-23) belongs to the IL-12 cytokine family, is a heterodimeric cytokine consisting of an IL12B (p40) subunit (shared with IL-12) and the IL23A (p19) subunit. IL-23 functions through binding to IL-23 receptor composed of IL-12R p1 and IL-23R (p19 subunit binds IL-23R while p40 subunit binds IL-12Rβ1), resulting in Janus kinase 2 and Tyrosine kinase 2 kinases recruitment and phosphorylation of STAT3 and STAT4, leading to gene activation. STAT3 is responsible for key Th17 development characteristics such as RORγt expression, or transcription of Th17 cytokines such as IL-17, IL-21, IL-22, and GM-CSF which mediate protection against fungi and bacteria and participate in barrier immunity. IL-23 is mainly secreted by activated dendritic cells, macrophages or monocytes stimulated by antigen stimulus. IL-23 receptor is expressed on Th17 and NK cells. It was found that autoimmune and cancerous diseases are associated with IL-23 imbalance and increase. The most important function of IL-23 is its role in the development and differentiation of effector Th17 cells. In the context of chronic inflammation, activated DCs and macrophages produce IL-23, which promotes the development of Th17 cells. Autoimmune diseases such as psoriasis, Crohn's disease, rheumatoid arthritis, or multiple sclerosis have recently been found to be associated with IL-23-mediated signaling promoted by IL-23 receptor-expressing TH-17 and other lymphocyte subsets.

Native human p19 (IL-23A) precursor polypeptide consists of 189 amino acid residues (amino acids 1-19 are signal peptide), while the mature polypeptide consists of 170 amino acid residues (SEQ ID NO: 73). Native human p40 (IL-12B) precursor polypeptide consists of 328 amino acid residues (amino acids 1-22 are signal peptide), while the mature polypeptide consists of 306 amino acid residues (SEQ ID NO: 62). In some embodiments, the IL-23 moiety (or IL-23 subunit) is a mature IL-23 (or IL-23 mature subunit). In some embodiments, the IL-23A (p19) or variant thereof is a polypeptide substantially homologous to amino acid sequence of a wild-type IL-23A (p19) (SEQ ID NO: 73), e.g., having at least about 85% (such as at least about any of 90%, 91%, 92%, 93%, 94%, 95%, 96%, 97%, 98%, 99%, or 100%) amino acid sequence identity to a wild-type IL-23A (p19) (SEQ ID NO: 73). In some embodiments, the IL-12B (p40) subunit or variant thereof is a polypeptide substantially homologous to amino acid sequence of a wild-type of IL-12B (p40) (SEQ ID NO: 62), e.g., having at least about 85% (such as at least about any of 90%, 91%, 92%, 93%, 94%, 95%, 96%, 97%, 98%, 99%, or 100%) amino acid sequence identity to a wild-type of IL-12B (p40) (SEQ ID NO: 62). In some embodiments, the IL-23 (or subunit) or variant thereof is not glycosylated. In some embodiments, the IL-23 (or subunit) or variant thereof is glycosylated. In some embodiments, the IL-23 variant comprises one wildtype subunit (e.g., wt p19) and one mutant subunit (e.g., variant p40). In some embodiments, the IL-23 variant comprises two variant subunits (p19 variant and p40 variant). In some embodiments, the IL-23 variant comprises two wildtype subunits (e.g., wt p19 and p40) that are linked together via a synthetic peptide linker (e.g., any of SEQ ID NOs: 227-229, 245, and 246) or a chemical linker. Within the p40 subunit, amino acid residues that are important for IL-23 receptor binding are C177, E45, E59, and D62 (Luo et al. J Mol Biol. 2010:402(5):797-812).

In some embodiments, the IL-23 moiety comprises a wildtype p19 subunit (SEQ ID NO: 73). In some embodiments, the IL-23 moiety comprises a variant p19 subunit. In some embodiments, the IL-23 moiety comprises a wildtype p40 subunit (SEQ ID NO: 62). In some embodiments, the IL-23 moiety comprises a variant p40 subunit. In some embodiments, the IL-23 moiety comprises a wildtype or variant p19 subunit and a wildtype or variant p40 subunit connected by a peptide linker (e.g., any of SEQ ID NOs: 227-229, 245, and 246). In some embodiments, the IL-23 moiety comprises from N-terminus to C-terminus: wildtype or variant p40 subunit-linker (e.g., any of SEQ ID NOs: 227-229, 245, and 246)-wildtype or variant p19 subunit. In some embodiments, the IL-23 moiety comprises from N-terminus to C-terminus: wildtype or variant p19 subunit-linker (e.g., any of SEQ ID NOs: 227-229, 245, and 246)-wildtype or variant p40 subunit. In some embodiments, the IL-23 variant comprises one or more mutations within the p40 subunit at a position selected from the group consisting of E45, Q56, V57, K58, E59, F60, G61, D62, A63, G64, Q65, and C177 relative to a wildtype p40 subunit (SEQ ID NO: 62). In some embodiments, the IL-23 variant comprises one or more mutations within the p40 subunit selected from the group consisting of Q56A, V57A, K58A, E59A, F60A, F60D, G61A, D62A, A63S, G64A, and Q65A relative to a wildtype p40 subunit (SEQ ID NO: 62). In some embodiments, the p40 subunit of the IL-23 variant comprises the sequence of any of SEQ ID NOs: 63-66 and 140. In some embodiments, the IL-23 variant comprises an E59A/F60A mutation within the p40 subunit relative to a wildtype p40 subunit (SEQ ID NO: 62). In some embodiments, the p40 subunit of the IL-23 variant comprises the sequence of SEQ ID NO: 63. In some embodiments, the IL-23 variant comprises an F60A mutation within the p40 subunit relative to a wildtype p40 subunit. In some embodiments, the p40 subunit of the IL-23 variant comprises the sequence of SEQ ID NO: 65. In some embodiments, the IL-23 variant comprises an F60D mutation within the p40 subunit relative to a wildtype p40 subunit (SEQ ID NO: 62). In some embodiments, the p40 subunit of the IL-23 variant comprises the sequence of SEQ ID NO: 140. In some embodiments, the p40 subunit and the p19 subunit of the IL-23 or variant thereof are connected by a linker (e.g., any of SEQ ID NOs: 227-229, 245, and 246). In some embodiments, the IL-23 variant comprises the sequence of SEQ ID NO: 75. In some embodiments, the IL-23 moiety is a recombinant “wildtype” IL-23 comprising a wildtype p35 subunit and a wildtype p40 subunit connected by a linker (e.g., any of SEQ ID NOs: 227-229, 245, and 246), such as comprising the sequence of SEQ ID NO: 74.

### IL-17

In some embodiments, the immunostimulatory cytokine or variant thereof is IL-17 or variant thereof. The IL-17 family comprises IL17A, IL-17B, IL-17C, IL-17D, IL-17E (a.k.a. IL-25) and IL-17F. Interleukin 17A (IL-17A or IL-17) is a disulfide-linked, homodimeric, secreted glycoprotein with a molecular mass of about 35 kDa. Each subunit of the homodimer is approximately 15-20 KDa. IL-17A is a pro-inflammatory cytokine produced by T helper 17 (Th17) cells in response to their stimulation with IL-23. IL-17 interacts with IL-17R and activates several signaling cascades that, in turn, lead to the induction of chemokines. These chemokines act as chemoattractant to recruit immune cells, such as monocytes and neutrophils to the site of inflammation.

### Target Molecules or Target Antigens

“Target antigen” or “target epitope” used herein can refer to any protein or polypeptide that can be specifically recognized by the antigen-binding protein, antigen-binding polypeptide, or antigen-binding fragment/domain described herein (can be used interchangeably), such as tumor antigen or epitope, pathogen antigen or epitope, antigen or epitope involved in autoimmune diseases, allergy, and/or graft rejection, ligand or receptor or portion thereof (e.g., extracellular domain of a ligand/receptor), immune cell surface antigen or epitope, etc. In some embodiments, the antigen-binding protein is monovalent and monospecific. In some embodiments, the antigen-binding protein is multivalent (e.g., bivalent) and monospecific. In some embodiments, the antigen-binding protein is multivalent (e.g., bivalent) and multispecific (e.g., bispecific). The valency and specificity of the antigen-binding protein herein is referring to valency and specificity of the antigen-binding fragment(s) (e.g., ligand, receptor, VHH, scFv, or Fab) of the immunocytokine, not including valency or specificity of the cytokine or variant thereof.

In some embodiments, the target antigen is a cell surface molecule (e.g., extracellular domain of a receptor/ligand). In some embodiments, the target antigen acts as a cell surface marker on a target cell (e.g., tumor cell, immune cell) associated with a special disease state. The target antigens (e.g., tumor antigen, extracellular domain of a receptor/ligand) specifically recognized by the antigen-binding domain may be antigens on a single diseased cell or antigens that are expressed on different cells that each contribute to the disease. The target antigens specifically recognized by the antigen-binding domain(s) may be directly or indirectly involved in the diseases.

### Tumor Antigen

In some embodiments, the target antigen or epitope (such as the third target molecule) is a tumor antigen or epitope.

Tumor antigens are proteins that are produced by tumor cells that can elicit an immune response, particularly T cell mediated immune responses. The selection of the targeted antigen of the invention will depend on the particular type of cancer to be treated. Exemplary tumor antigens include, for example, a glioma-associated antigen, BCMA (B-cell maturation antigen), carcinoembryonic antigen (CEA), β-human chorionic gonadotropin, alpha-fetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, and mesothelin. In some embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens is onco-fetal antigens such as carcinoembryonic antigen (CEA). In B-cell lymphoma, the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells. Non-limiting examples of TSA or TAA antigens include the following: differentiation antigens such as MART-1/MelanA (MART-1), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4 (TPBG), 791Tgp72, alpha-fetoprotein, beta-HCG, BCA225, BTAA, CA 125, CA 15-3\CA 27.29\BCAA, CA 195, CA 242, CA-50, CAM43, CD68\Pl, CO-029, FGF-5, G250, Ga733\EpCAM, HTgp-175, M344, MA-50, MG7-Ag, MOV18, NB/70K, NY-CO-1, RCAS 1, SDCCAGI6, TA-90\Mac-2 binding protein\cyclophilin C-associated protein, TAAL6, TAG72, TLP, and TPS.

In some embodiments, the tumor antigen is selected from the group consisting of FIXa, FX, DLL3, DLL4, Ang-2, Nectin-4, FOLRa, GPNMB, CD56 (NCAM), TACSTD2 (TROP-2), tissue factor, ENPP3, P-cadherin, STEAP1, CEACAM5, Mucin 1 (Sialoglycotope CA6), Guanylyl cyclase C (GCC), SLC44A4, LIV1 (ZiP6), NaPi2b, SLITRK6, SC-16, fibronectin, extra-domain B (EDB), Endothelium receptor ETB, ROBO4, Collagen IV, Periostin, Tenascin c, CD74, CD98, Mesothelin, TSHR, CD19, CD123, CD22, CD30, CD171, CS-1, CLL-1, CD33, EGFRvIII, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2 (CD309), LewisY, CD24, platelet-derived growth factor receptor-beta (PDGFR-beta), SSEA-4, CD20, Folate receptor alpha, ERBB2 (HER2/neu), MUC1, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEMI/CD248, TEM7R, CLDN6, CLDN18.2, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL 1. In some embodiments, the tumor antigen is selected from the group consisting of BCMA, EphA2, HER2, GD2, Glypican-3, 5T4, 8H9, αᵥβ₆ integrin, B7-H3, B7-H6, CAIX, CA9, CD19, CD20, CD22, CD30, CD33, CD38, CD44, CD44v6, CD44v7/8, CD70 (TNFSF7), CD123, CD138, CD171, CEA, CSPG4, EGFR, EGFRvIII, EGP2, EGP40, EpCAM, ERBB3, ERBB4, ErbB3/4, FAP, FAR, FBP, fetal AchR, Folate Receptor a, GD2, GD3, HLA-AI MAGE A1, HLA-A2, IL11Ra, IL13Ra2, KDR, Lewis-Y, MCSP, Mesothelin, Muc1, Muc16, NCAM, NKG2D ligands, NY-ESO-1, PRAME, PSCA, PSCl, PSMA, ROR1, SURVIVIN, TAG72, TEMI, TEMS, VEGFR2, carcinoembryonic antigen, and HMW-MAA. Also see exemplary tumor antigens described in Shim H. (Biomolecules. 2020 March; 10(3): 360), and Diamantis N. and Banerji U. Br J Cancer. 2016; 114(4): 362-367, the contents of which are incorporated herein by reference in their entirety.

In some embodiments, the tumor antigen is HER2. In some embodiments, the third binding domain specifically recognizing HER2 is derived from trastuzumab (e.g., Herceptin®), pertuzumab (e.g., Perjeta®), margetuximab, or 7C2. In some embodiments, the third binding domain specifically recognizing HER2 comprises heavy chain CDRs, light chain CDRs, or all 6 CDRs of any of trastuzumab, pertuzumab, margetuximab, or 7C2. In some embodiments, the third binding domain specifically recognizing HER2 comprises VH and/or VL of trastuzumab, pertuzumab, margetuximab, or 7C2. In some embodiments, the immunocytokine comprises a parental anti-HER2 antibody (e.g., full-length antibody).

### Pathogen Antigen

In some embodiments, the target antigen or epitope (e.g., the third target molecule) is a pathogen antigen or epitope, such as a fungal, viral, bacterial, protozoal or other parasitic antigen or epitope.

In some embodiments, the fungal antigen is from Aspergillus or Candida. Fungal antigens for use with compositions and methods of the invention include, but are not limited to, e.g., Candida fungal antigen components; Aspergillus fungal antigens; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Bacterial antigens for use with the immunocytokine disclosed herein include, but are not limited to, e.g., bacterial antigens such as pertussis toxin, filamentous hemagglutinin, pertactin, FIM2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens. Partial or whole pathogens may also be: Haemophilus influenza; Plasmodium falciparum; Neisseria meningitidis; Streptococcus pneumoniae; Neisseria gonorrhoeae; Salmonella serotype typhi; Shigella; Vibrio cholerae; Dengue Fever; Encephalitides; Japanese Encephalitis; lyme disease; Yersinia pestis; west nile virus; yellow fever; tularemia; hepatitis (viral; bacterial); RSV (respiratory syncytial virus); HPIV 1 and HPIV 3; adenovirus; smallpox; allergies and cancers.

Examples of protozoal and other parasitic antigens include, but are not limited to, e.g., Plasmodium falciparum antigens such as merozoite surface antigens, sporozoite surface antigens, circumsporozoite antigens, gametocyte/gamete surface antigens, blood-stage antigen pf 155/RESA and other plasmodial antigen components; Toxoplasma antigens such as SAG-1, p30 and other toxoplasmal antigen components; schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen and other trypanosomal antigen components.

In some embodiments, the viral antigen is from Herpes simplex virus (HSV), respiratory syncytial virus (RSV), metapneumovirus (hMPV), rhinovirus, parainfluenza (PIV), Epstein-Barr virus (EBV), Cytomegalovirus (CMV), JC virus (John Cunningham virus), BK virus, HIV, Zika virus, human coronavirus, norovirus, encephalitis virus, or Ebola. In some embodiments, the virus is an Orthomyxoviridae virus selected from the group consisting of Influenza A virus, Influenza B virus, Influenza C virus, and any subtype or reassortant thereof. In some embodiments, the virus is an Influenza A virus or any subtype or reassortant thereof, such as Influenza A virus subtype H1N1 (H1N1) or Influenza A virus subtype H5N1 (H5N1). In some embodiments, the virus is a Coronaviridae virus selected from the group consisting of alpha coronaviruses 229E (HCoV-229E), New Haven coronavirus NL63 (HCoV-NL63), beta coronaviruses OC43 (HCoV-OC43), coronavirus HKU1 (HCoV-HKU1), Severe Acute Respiratory Syndrome coronavirus (SARS-CoV), Middle East Respiratory Syndrome coronavirus (MERS-CoV), and Severe Acute Respiratory Syndrome Coronavirus 2 (SARS-CoV-2). In some embodiments, the virus is SARS-CoV, MERS-CoV, or SARS-CoV-2. In some embodiments, the virus is a Filoviridae virus selected from Ebola virus (EBOV) and Marburg virus (MARV). In some embodiments, the virus is a Flaviviridae virus selected from the group consisting of Zika virus (ZIKV), West Nile virus (WNV), Dengue virus (DENV), and Yellow Fever virus (YFV).

### Antigens Involved in Autoimmune Diseases, Allergy, and Graft Rejection

In some embodiments, the target antigen or epitope (e.g., the third target molecule) is an antigen or epitope involved in autoimmune diseases, allergy, and/or graft rejection. For example, an antigen involved in any one or more of the following autoimmune diseases or disorders can be used in the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, inflammatory bowel disease (IBD), Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatibility antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease. In some embodiments, the target antigen is CD3, CD4, CD123, or CD8.

### Immune Checkpoint Molecule

In some embodiments, the target antigen or epitope (e.g., the first, second, and/or third target molecule) is an immune checkpoint molecule. Immune checkpoints are regulators of the immune system.

In some embodiments, the immune checkpoint molecule is a stimulatory immune checkpoint molecule. In some embodiments, the stimulatory immune checkpoint molecule is selected from the group consisting of CD27, CD28, OX40, ICOS, GITR, 4-1BB, CD27, CD40, CD3, and HVEM. Thus, in some embodiments, the first binding domain described herein is an activator of a stimulatory immune checkpoint molecule, which can stimulate, activate, or increase the intensity of an immune response mediated by a stimulatory immune checkpoint molecule. The antibody or antigen-binding fragment described herein can be derived from any antibody known in the art that activates a stimulatory immune checkpoint molecule. In some embodiments, the first binding domain is a ligand or receptor of a stimulatory immune checkpoint molecule, e.g., can activate stimulatory immune checkpoint signaling. In some embodiments, the second binding domain (e.g., antibody, antigen-binding domain, or ligand/receptor-Fc fusion protein) described herein is an antagonist of a stimulatory immune checkpoint molecule, which can reduce or block the intensity of an immune response mediated by a stimulatory immune checkpoint molecule.

In some embodiments, the immune checkpoint molecule is an inhibitory immune checkpoint molecule. In some embodiments, the inhibitory immune checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, and VISTA. In some embodiments, the inhibitory immune checkpoint molecule is PD-1, PD-L2, or PD-L1. In some embodiments, the inhibitory immune checkpoint molecule is CTLA-4. In some embodiments, the inhibitory immune checkpoint molecule is TIGIT. Thus, in some embodiments, the antigen-binding protein (e.g., antibody, antigen-binding domain, or ligand/receptor-Fc fusion protein) described herein is an immune checkpoint inhibitor, which totally or partially reduces, inhibits, or interferes with one or more inhibitory immune checkpoint molecules. The antibody or antigen-binding domain described herein can be derived from any antibody known in the art that serves as an immune checkpoint inhibitor. In some embodiments, the antigen-binding fragment is a ligand (e.g., CD155, PD-L2 or PD-L1) or receptor of an inhibitory immune checkpoint molecule (e.g., TIGIT or PD-1), e.g., can activate or stimulate an inhibitory immune checkpoint signaling (e.g., TIGIT or PD-1 signaling). In some embodiments, the antigen-binding protein (e.g., antibody, antigen-binding domain, or ligand/receptor-Fc fusion protein) described herein is an agonist of an inhibitory immune checkpoint molecule, which can stimulate, activate, or increase the intensity of an immune response mediated by an inhibitory immune checkpoint molecule.

### Cell Surface Ligand or Receptor

In some embodiments, the target antigen or epitope (e.g., the third target molecule) is a ligand or receptor or portion thereof, such as extracellular domain of a ligand/receptor. In some embodiments, the ligand or receptor is derived from a molecule selected from the group consisting of IL-2, IL-2Rα (CD25), IL-3Rα (CD123), PD-1, PD-L1, PD-L2, CD155, NKG2A, NKG2C, NKG2F, NKG2D, BCMA, APRIL, BAFF, IL-3, IL-13, LLT1, AICL, DNAM-1, and NKp80. In some embodiments, the ligand is derived from APRIL and/or BAFF, which can bind to BCMA. In some embodiments, the receptor is an FcR and the ligand is an Fc-containing molecule. In some embodiments, the FcR is an Fcγ receptor (FcγR). In some embodiments, the FcγR is selected from the group consisting of FcγRIA (CD64A), FcγRIB (CD64B), FcγRIC (CD64C), FcγRIIA (CD32A), FcγRIIB (CD32B), FcγRIIIA (CD16a), and FcγRIIIB (CD16b).

The receptor of IL-2, interleukin-2 receptor (IL-2R), is a heterotrimeric protein expressed on the surface of certain immune cells, such as lymphocytes. TL-2R has three forms generated by different combinations of α chain (IL-2Rα, CD25, Tac antigen), β chain (IL-2Rβ, CD122), and γ chain (IL-2Rγ, γ_{c}, common gamma chain, or CD132). IL-2Rα binds IL-2 with low affinity, and the complex of IL-2Rβ and IL-2Rγ binds IL-2 with intermediate affinity, primarily on memory T cells and NK cells. The complex of all α, β, and γ chains bind IL-2 with high affinity on activated T cells and regulatory T cells (Tregs). CD25 (IL-2Rα) plays a critical role in the development and maintenance of Tregs, and may play a role in Treg expression of CD62L, which is required for the entry of Tregs into lymph nodes (Malek and Bayer, 2004). CD25 is a marker for activated T cells and Treg.

### Immune Cell Surface Antigen

In some embodiments, the target antigen or epitope (e.g., the third target molecule) is an immune cell surface antigen or epitope. Immune cells have different cell surface molecules. For example CD3 is a cell surface molecule on T-cells, whereas CD16, NKG2D, or NKp30 are cell surface molecules on NK cells, and CD3 or an invariant T-cell receptor (TCR) are the cell surface molecules on NKT-cells. In some embodiments, wherein the immune cell is a T-cell, the activation molecule is one or more of CD3, e.g., CD3ε, CD3δ, or CD3γ; or CD2, CD4, CD8, CD27, CD28, CD40, CD134, CD137, CD278, inhibitory immune checkpoint molecules (e.g., CTLA-4, PD-1, TIM3, BTLA, VISTA, LARG-3, or TIGIT), and stimulatory immune checkpoint molecules (CD27, CD28, CD137, OX40, GITR, or HVEM). In some embodiments, wherein the immune cell is a B cell, the cell surface molecule is CD19, CD20, or CD138. In other some embodiments, wherein the immune cell is a NK cell, the cell surface molecule is CD16, CD56 (NCAM), NKp46, NKp44, CD244, CD226, TIGIT, CD96, LAG3, TIM3, PD-1, KLRG1, CD161, CD94/NKG2, KIR, NKG2D, or NKp30. In some embodiments, wherein the immune cell is a NKT-cell, the cell surface molecule is CD3 or an invariant TCR. In some embodiments, wherein the immune cell is a myeloid dendritic cell (mDC), the cell surface molecule is CD11c, CD11b, CD13, CD45RO, or CD33. In some embodiments, wherein the immune cell is a plasma dendritic cell (pDC), the cell surface molecule is CD123, CD62L, CD45RA, or CD36. In some embodiments, wherein the immune cell is a macrophage, the cell surface molecule is CD163 or CD206. In some embodiments, the immune cell is selected from the group consisting of a monocyte, a dendritic cell, a macrophage, a B cell, a killer T cell (T_{c}, cytotoxic T lymphocyte, or CTL), a helper T cell (Tₕ), a regulatory T cells (Treg), a γδ T cell, a natural killer T (NKT) cell, and a natural killer (NK) cell.

In some embodiments, the immune cell surface antigen is selected from the group consisting of CD3 (e.g., CD3ε, CD3δ, CD3γ), CD4, CD5, CD8, CD16, CD27, CD28, CD40, CD64, CD89, CD134, CD137, CD278, NKp46, NKp30, NKG2D, TCRα, TCRβ, TCRγ, and TCRδ. In some embodiments, the immune cell surface antigen is CD3, CD4, or CD8.

Exemplary anti-CD4 antibodies include, but are not limited to, Ibalizumab (e.g., Trogarzo®), MAX.16H5, and IT1208. Exemplary anti-CD3 antibodies include, but are not limited to OKT3. Exemplary anti-CD8 antibodies include, but are not limited to, G10-1, OKT8, YTC182.20, 4B 11, and DK25.

### Activities of Binding Domains or Cytokines or Variants Thereof

The “activity” of a binding domain (e.g., to its target molecule) described herein comprises the binding affinity of the binding domain to corresponding target molecule; and/or the biological activity (or bioactivity) of the binding domain (e.g., cytokine or a variant thereof), such as inducing or inhibiting signal transduction, inducing or inhibiting cell proliferation, differentiation, and/or activation, inducing or inhibiting the secretion of effecting cytokine(s) (e.g., pro-inflammatory cytokines), inducing or inhibiting cytotoxicity against a tumor cell, inducing or inhibiting infectious agent elimination etc., upon binding domain/its target molecule binding. These biological activities are also referred to herein as direct biological activities. In some embodiments, the biological activity of a binding domain (e.g., to its target molecule) also comprises indirect biological activities, such as any biological activity resulting from the direct biological activities.

The “activity” of a cytokine or a variant thereof described herein comprises the binding affinity of the cytokine or a variant thereof to corresponding cytokine receptor; and/or the biological activity (or bioactivity) of the cytokine or a variant thereof, such as inducing or inhibiting signal transduction, inducing or inhibiting cell proliferation, differentiation, and/or activation, inducing or inhibiting the secretion of effecting cytokine(s) (e.g., pro-inflammatory cytokines), etc., upon cytokine/cytokine receptor binding. These biological activities are also referred to herein as direct biological activities. In some embodiments, the biological activity of a cytokine or a variant thereof also comprises indirect biological activities, such as any biological activity resulting from the direct biological activities. For example, in some embodiments, the biological activity also comprises cancer cell killing by immune cells attracted to the tumor site due to the secreted effecting cytokines, such as inflammatory markers IL-6, MIP-2 (GRO-β)/CXCL2, G-CSF/CSF3, TIMP-1, KC (GRO-α)/CXCL1, etc.

In some embodiments, the first binding domain or portion thereof is positioned at a hinge region (at N′ of hinge, C′ of hinge, or within hinge) between the second binding domain or portion thereof and an Fc domain subunit or portion thereof of the immunomodulatory molecule. In some embodiments, in the presence of binding of the second binding domain (e.g., ligand, receptor, VHH, scFv, or Fab) of the immunomodulatory molecule described herein to the second target antigen, the activity (binding affinity to first target molecule such as cytokine receptor, and/or biological activity) of the first binding domain (e.g., immunostimulatory cytokine or variant thereof) increases at least about 20% (such as at least about any of 30%, 40%, 50%, 60%, 70%, 80%, 90%, 100%, 200%, 300%, 400%, 500%, or more) compared to that in the absence of binding of the second binding domain to the second target molecule. In some embodiments, in the presence of binding of the second binding domain (e.g., ligand, receptor, VHH, scFv, or Fab) of the immunomodulatory molecule described herein to the second target molecule, the activity (binding affinity to the first target molecule such as cytokine receptor, and/or biological activity) of the first binding domain (e.g., immunostimulatory cytokine or variant thereof) increases to at least about 2-fold (such as at least about any of 3, 4, 5, 6, 7, 8, 9, 10, 20, 30, 40, 50, 60, 70, 80, 90, or 100-fold) of that in the absence of binding of the second binding domain to the second target molecule.

In some embodiments, in the absence of binding of the second binding domain (e.g., ligand, receptor, VHH, scFv, or Fab) of the immunomodulatory molecule described herein to the second target antigen, the activity (binding affinity to the first target molecule such as cytokine receptor, and/or biological activity) of the first binding domain (e.g., immunostimulatory cytokine or variant thereof) positioned at the hinge region of the antigen-binding polypeptide (such as positioned at the hinge region of a heavy chain of an antibody (e.g., full-length antibody), or positioned at the hinge region between the second binding domain (e.g., ligand, receptor, VHH, scFv, or Fab) and an Fc domain subunit (or portion thereof), see FIGS. 1A-1D, 1G, 1H, 1L-1O) is no more than about 70% (such as no more than about any of 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding first binding domain (e.g., immunostimulatory cytokine or variant thereof) in a free state.

In some embodiments, the “corresponding first binding domain” (e.g., “corresponding cytokine or variant thereof”) is the same as the first binding domain (e.g., cytokine or variant thereof) positioned at the hinge region, but expressed under a different state or at a different position. A first binding domain (e.g., cytokine or variant thereof) “in a free state” herein refers to a first binding domain (e.g., cytokine or variant thereof) in a soluble form, without attaching to any moiety such as cell membrane or another molecule (e.g., Fc fragment, or N-terminus or C-terminus of a full-length antibody or antigen binding fragment (e.g., ligand, receptor, VHH, scFv, or Fab)).

In some embodiments, in the absence of binding of the second binding domain of a full-length antibody to the second target antigen, the activity (binding affinity to first target molecule such as cytokine receptor or subunit thereof, and/or biological activity) of the first binding domain (e.g., cytokine or variant thereof) positioned at the hinge region of a heavy chain of the full-length antibody is no more than about 50% (such as no more than about any of 40%, 30%, 20%, 10%, 9/, 8%, 7%, 6%, 5%, 4%, 3%, 2%, 1%, 0.9%,0.8%, 0.7%, 0.6%, 0.5%, 0.4%, 0.3%, 0.2%, 0.1%, or 0%) of that of a corresponding first binding domain (e.g., cytokine or variant thereof) expressed at any of: i) the N-terminus of a VH of the full-length antibody, ii) the N-terminus of a VL of the full-length antibody, iii) the C-terminus of a heavy chain of the full-length antibody, iv) the C-terminus of a CL of the full-length antibody, and v) the N-terminus of an Fc domain subunit of the full-length antibody. In some embodiments, in the absence of binding of the second binding domain (e.g., scFv or Fab) to the second target molecule, the activity (binding affinity to first target molecule such as cytokine receptor, and/or biological activity) of the first binding domain (e.g., cytokine or variant thereof) positioned at the hinge region between the second binding domain (e.g., scFv or Fab) and an Fc domain subunit (or portion thereof) is no more than about 50% (such as no more than about any of 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding first binding domain (e.g., cytokine or variant thereof) expressed at any of: i) the N-terminus of a VH of the second binding domain (e.g., scFv or Fab), ii) the N-terminus of a VL of the second binding domain (e.g., scFv or Fab), iii) the C-terminus of the Fc domain subunit (or portion thereof), iv) the C-terminus of a CL of the second binding domain (Fab), and v) the N-terminus of the Fc domain subunit. In some embodiments, in the absence of binding of the second binding domain (e.g., VHH, ligand, or receptor) to the second target antigen, the activity (binding affinity to the first target molecule such as cytokine receptor or subunit thereof, and/or biological activity) of the first binding domain (e.g., cytokine or variant thereof) positioned at the hinge region between the second binding domain (e.g., VHH, ligand, or receptor) and an Fc domain subunit (or portion thereof) is no more than about 50% (such as no more than about any of 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0%) of that of a corresponding first binding domain (e.g., cytokine or variant thereof) expressed at any of: i) the N-terminus of the second binding domain (e.g., VHH, ligand, or receptor), ii) the C-terminus of the Fc domain subunit (or portion thereof), and iii) the N-terminus of the Fc domain subunit.

In some embodiments, in the presence of binding of the second binding domain of a full-length antibody to the second target molecule, the activity (binding affinity to first target molecule such as cytokine receptor or subunit thereof, and/or biological activity) of the first binding domain (e.g., cytokine or variant thereof) positioned at the hinge region of a heavy chain of the full-length antibody is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding first binding domain (e.g., cytokine or variant thereof) expressed at any of: i) the N-terminus of a VH of the full-length antibody, ii) the N-terminus of a VL of the full-length antibody, iii) the C-terminus of a heavy chain of the full-length antibody, iv) the C-terminus of a CL of the full-length antibody, and v) the N-terminus of an Fc subunit of the full-length antibody. In some embodiments, in the presence of binding of the second binding domain (e.g., scFv or Fab) to the second target molecule, the activity (binding affinity to first target molecule such as cytokine receptor or subunit thereof, and/or biological activity) of the first binding domain (e.g., cytokine or variant thereof) positioned at the hinge region between the second binding domain (e.g., scFv or Fab) and an Fc domain subunit (or portion thereof) is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding first binding domain (e.g., cytokine or variant thereof) expressed at any of: i) the N-terminus of a VH of the second binding domain (e.g., scFv or Fab), ii) the N-terminus of a VL of the second binding domain (e.g., scFv or Fab), iii) the C-terminus of the Fc domain subunit (or portion thereof), iv) the C-terminus of a CL of the second binding domain (Fab), and v) the N-terminus of the Fc domain subunit. In some embodiments, in the presence of binding of the second binding domain (e.g., VHH, ligand, or receptor) to the second target molecule, the activity (binding affinity to first target molecule such as cytokine receptor or subunit thereof, and/or biological activity) of the first binding domain (e.g., cytokine or variant thereof) positioned at the hinge region between the second binding domain (e.g., VHH, ligand, or receptor) and an Fc domain subunit (or portion thereof) is at least about 70% (such as at least about any of 80%, 90%, 100%, 110%, 120%, 130%, 140%, 150%, 160%, 170%, 180%, 190%, 200%, 300%, 400%, 500%, or more) of that of a corresponding first binding domain (e.g., cytokine or variant thereof) expressed at any of; i) the N-terminus of the second binding domain (e.g., VHH, ligand, or receptor), ii) the C-terminus of the Fc domain subunit (or portion thereof), and iii) the N-terminus of the Fc domain subunit.

In some embodiments, the first binding domain is a ligand or variand thereof. In some embodiments, the first binding domain is a cytokine (e.g., immunostimulatory cytokine) or variand thereof. In some embodiments, the immunostimulatory cytokine is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF. In some embodiments, the activity (binding affinity to corresponding cytokine receptor or subunit thereof, and/or biological activity) of the cytokine variant in a free state is no more than about 80% (such as no more than about any of 70%, 60%, 50%, 40%, 30%, 20%, 10%, or 5%) of that of a corresponding wildtype cytokine in a free state. In some embodiments, the activity (binding affinity to corresponding cytokine receptor or subunit thereof, and/or biological activity) of the cytokine variant in a free state is the same or similar (such as within about 20% difference) of that of a corresponding wildtype cytokine in a free state. In some embodiments, the cytokine or variant thereof is a cytokine variant. In some embodiments, the first binding domain is an immunostimulatory cytokine variant, and wherein the activity (binding affinity to first target molecule such as corresponding cytokine receptor or subunit thereof, and/or biological activity) of the immunostimulatory cytokine variant in a free state is no more than about 80% (such as no more than about any of 70%, 60%, 50%, 40%, 30%, 20%, 10%, or 5%) of that of a corresponding wildtype immunostimulatory cytokine in a free state.

In some embodiments, in the absence of binding of the second binding domain (e.g., ligand, receptor, VHH, scFv, or Fab) of the immunomodulatory molecule described herein to the second target molecule, the activity (binding affinity to first target molecule such as corresponding cytokine receptor or subunit thereof, and/or biological activity) of the first binding domain (e.g., cytokine variant) positioned at the hinge region of the antigen-binding polypeptide (such as positioned at the hinge region of a heavy chain of the antibody (e.g., full-length antibody), or positioned at the hinge region between an second binding domain (e.g., ligand, receptor, VHH, scFv, or Fab) and an Fc domain subunit (or portion thereof)) is no more than about 80% (such as no more than about any of 70%, 60%, 50%, 40%, 30%, 20%, 10%, 9%, 8%, 7%, 6%, 5%, 4%, 3%, 2%, 1%, 0.9%,0.8%, 0.7%, 0.6%, 0.5%, 0.4%, 0.3%, 0.2%, 0.1%, or 0%) of that of a corresponding wildtype or non-variant first binding domain (e.g., wildtype cytokine, or a corresponding recombinant “wildtype” cytokine expressed in the same format but comprising wildtype subunits) positioned at the same region. For example, in some embodiments, the IL-12 variant comprises from N-terminus to C-terminus: variant p40 subunit-linker-wildtype p35 subunit, and the corresponding recombinant “wildtype” IL-12 comprises from N-terminus to C-terminus: wildtype p40 subunit-linker-wildtype p35 subunit. In some embodiments, the cytokine variant is an IL-2 variant, and the corresponding wildtype cytokine is a “wildtype” IL-2.

In some embodiments, in the presence of binding of the second binding domain (e.g., ligand, receptor, VHH, scFv, or Fab) of the immunomodulatory molecule described herein to the second target molecule, the activity (binding affinity to first target molecule such as corresponding cytokine receptor or subunit thereof, and/or biological activity) of the first binding domain (e.g., cytokine variant) positioned at the hinge region of the antigen-binding polypeptide (such as positioned at the hinge region a heavy chain of an antibody (e.g., full-length antibody), or positioned at the hinge region between an second binding domain (e.g., ligand, receptor, VHH, scFv, or Fab) and an Fc domain subunit (or portion thereof)) is at least about 1% (such as at least about any of 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, 200%, or more) of that of a corresponding wildtype or non-variant first binding domain (e.g., wildtype cytokine, or a corresponding recombinant “wildtype” cytokine expressed in the same format but comprising wildtype subunits) positioned at the same region.

In some embodiments, in the absence of binding of the second binding domain (e.g., ligand, receptor, VHH, scFv, or Fab) of the immunomodulatory molecule described herein to the second target molecule, the activity (binding affinity to first target molecule such as corresponding cytokine receptor or subunit thereof, and/or biological activity) of the first binding domain (e.g., cytokine variant) positioned at the hinge region of the antigen-binding polypeptide (such as positioned at the hinge region of a heavy chain of an antibody (e.g., full-length antibody), or positioned at the hinge region between an second binding domain (e.g., ligand, receptor, VHH, scFv, or Fab) and an Fc domain subunit (or portion thereof)) is no more than about 80% (such as no more than about any of 70%, 60%, 50%, 40%, 30%, 20%, 10%, 9%, 8%, 7%, 6%, 5%, 4%, 3%, 2%, 1%, 0.9%,0.8%, 0.7%, 0.6%, 0.5%, 0.4%, 0.3%, 0.2%, 0.1%, or 0%) of that of a corresponding wildtype or non-variant first binding domain (e.g., wildtype cytokine, or a corresponding recombinant “wildtype” cytokine in the same format but comprising wildtype subunits) positioned at the same region; and in the presence of binding of the second binding domain (e.g., ligand, receptor, VHH, scFv, or Fab) of the immunomodulatory molecule described herein to the second target molecule, the activity (binding affinity to first target molecule such as corresponding cytokine receptor or subunit thereof, and/or biological activity) of the first binding domain (e.g., cytokine variant) positioned at the hinge region of the antigen-binding polypeptide (such as positioned at the hinge region of a heavy chain of an antibody (e.g., full-length antibody), or positioned at the hinge region between an second binding domain (e.g., ligand, receptor, VHH, scFv, or Fab) and an Fc domain subunit (or portion thereof)) is at least about 1% (such as at least about any of 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, 200%, or more) of that of a corresponding wildtype or non-variant first binding domain (e.g., wildtype cytokine, or a corresponding recombinant “wildtype” cytokine in the same format but comprising wildtype subunits) positioned at the same region.

### Binding Affinity

Binding affinity of a molecule (e.g., cytokine moiety, immunomodulatory molecule comprising a cytokine moiety, or binding domain) and its binding partner (e.g., cytokine receptor or subunits thereof, or target molecule) can be determined experimentally by any suitable ligand binding assays or antibody/antigen binding assays known in the art, e.g., Western blots, sandwich enzyme-linked immunosorbent assay (ELISA), Meso Scale Discovery (MSD) electrochemiluminescence, bead based multiplex immunoassays (MIA), RIA, Surface Plasma Resonance (SPR), ECL, IRMA, FACS, EIA, Biacore assay, Octet analysis, peptide scans, etc. For example, easy analysis is possible by using the cytokine or variant thereof, immunomodulatory molecule comprising the cytokine or variant thereof, or its corresponding receptor or subunits thereof marked with a variety of marker agents, as well as by using BiacoreX (made by Amersham Biosciences), which is an over-the-counter, measuring kit, or similar kit, according to the user's manual and experiment operation method attached with the kit.

In some embodiments, protein microarray is used for analyzing the interaction, function and activity of the binding domain (e.g., cytokine moiety) described herein to its corresponding target molecule (e.g., cytokine receptor), on a large scale. The protein chip has a support surface bound with a range of capture proteins (e.g., cytokine receptor or subunits thereof). Fluorescently labeled probe molecules (e.g., cytokine moiety or immunomodulatory molecule described herein) are then added to the array and upon interaction with the bound capture protein, a fluorescent signal is released and read by a laser scanner.

In some embodiments, the binding affinity of a binding domain (e.g., cytokine moiety) or immunomodulatory molecule described herein and its corresponding target molecule (e.g., cytokine receptor or subunit thereof) is measured using SPR (Biacore T-200). For example, anti-human antibody is coupled to the surface of a CM-5 sensor chip (e.g., using EDC/NHS chemistry). Then a human cytokine receptor-Fc fusion protein (e.g., IL-2Rα-Fc, IL-2Rβ-Fc, IL-2Rγ-Fc) is used as the captured ligand over this surface. Serial dilutions of immunomodulatory molecule comprising a cytokine moiety (e.g., IL-2 variant) are allowed to bind to the captured ligands (free state IL-2 variant serves as control), and the response units (RU) can be plotted against immunomodulatory molecule concentration to determine EC50 values, or plotted against time to monitor the binding and dissociation of immunomodulatory molecule to cytokine receptor-Fc in real time. Equilibrium dissociation constant (K_{D}) and dissociation rate constant can be determined by performing kinetic analysis using Biacore evaluation software. The binding affinity of each test immunomodulatory molecule to the cytokine receptor can be calculated as percentage relative to that of a corresponding free state cytokine moiety. In some embodiments, a cell line expressing a cytokine receptor (e.g., IL-2R) on the cell surface is incubated with an immunomodulatory molecule comprising a cytokine moiety (e.g., IL-2 variant) described herein, after incubation, the cells are washed, then an anti-IgG-conjugated with fluorescent protein (e.g., APC) is added to detect binding affinity of the immunomodulatory molecule to the cells, such as by FACS.

In some embodiments, the K_{D} of the binding between the binding domain (e.g., cytokine or variant thereof) in free state and its corresponding target molecule (e.g., cytokine receptor or subunits thereof) is about any of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, in the absence of binding of the second binding domain (e.g., ligand, receptor, VHH, scFv, or Fab) of the immunomodulatory molecule described herein to the second target molecule, the K_{D} of the binding between the first binding domain (e.g., cytokine or variant thereof) positioned at the hinge region of the antigen-binding polypeptide (such as positioned at the hinge region of a heavy chain of the antibody (e.g., full-length antibody), or positioned at the hinge region between an second binding domain (e.g., ligand, receptor, VHH, scFv, or Fab) and an Fc domain subunit (or portion thereof)) and its corresponding first target molecule (e.g., cytokine receptor or subunits thereof) is undetectable (e.g., no binding), or the KD is higher than (i.e., binds weaker than) that in the presence of binding of the second binding domain (e.g., ligand, receptor, VHH, scFv, or Fab) of the immunomodulatory molecule described herein to the second target molecule.

### Biological Activity

Various methods for determining the biological activities (or bioactivities) of binding domains (e.g., cytokines or variants thereof), or immunomodulatory molecules described herein are described in the art, such as bioassays. Any antigen/antibody binding, ligand/receptor binding, or cytokine assays known in the art can be adapted to test bioactivities of binding domains (e.g., cytokine moieties) or immunomodulatory molecules described herein.

For example, a bioassay focuses on biological activity of cytokines or ligands/receptors and using it as a read out. In a bioassay, the activity of a sample is tested on a sensitive cell line (e.g., primary cell cultures or in vitro adapted cell lines that are dependent and/or responsive to the test sample) and the results of this activity (e.g., cellular proliferation) are compared to a standard cytokine preparation. Other aspects of biological activity of cytokines include induction of further cytokine secretion, induction of killing, antiviral activity, degranulation, cytotoxicity, chemotaxis, and promotion of colony formation. In vitro assays to measure all of these activities are available. See, e.g., “Cytokine Bioassays” of Bioassays—BestProtocols®, from eBioscience® (http://tools.thermofisher.com/content/sfs/manuals/cytokine-bioassays.pdf), the content of which is incorporated herein by reference in its entirety.

For example, in a cytokine-induced proliferation assay, samples (e.g., IL-2 moiety or IL-2 immunomodulatory molecule) and standard (e.g., IL-2 in free state) are diluted via serial dilution in an assay plate filled with culture medium, indicator cells (e.g., CTLL-2, or PBMC stimulated with anti-CD3 Ab) are washed and resuspend in culture medium then added into each well. The cells are incubated for sufficient time (e.g., 24 hours or longer) at 37° C., 5% CO₂ in a humidified incubator. Then cell viability test agents (e.g., resazurin, MTT assay agents) can be added to the plate and allow for sufficient incubation, then read with spectrophotometer. The EC50 values (concentration of test sample required to exhibit 50% of maximal response) for cell proliferation can then be obtained from non-linear regression analysis of dose-response curves. Cell number can also be counted under microscope, and compare to that treated with standard or control. For another example, in a cytokine-induced cytokine production assay, samples (e.g., IL-12 or IL-23 moiety, or IL-12 or IL-23 immunomodulatory molecule) and standard (e.g., IL-12 or IL-23 in free state) are diluted via serial dilution in an assay plate filled with culture medium, indicator cells (e.g., splenocytes, activated CD4+ T cells, or activated CD8+ T cells) are washed and resuspend in culture medium then added into each well. Cells are incubated for sufficient time (e.g., 24-48 hours) at 37° C., 5% CO₂ in a humidified incubator, then supernatants are harvest for determination of cytokine expression by ELISA, following ELISA protocol for target cytokine of interest (e.g., IFN-γ). For another example, in a cytokine-induced cell surface marker expression assay, samples (e.g., IFN-γ moiety, or IFN-γ immunomodulatory molecule) and standard (e.g., IFN-γ in free state) are diluted via serial dilution in an assay plate filled with culture medium, indicator cells (e.g., HEK-Blue™ IFN-γ cells) are washed and resuspend in culture medium then added into each well. Cells are incubated for sufficient time (e.g., 24-48 hours) at 37° C., 5% CO₂ in a humidified incubator, then cell surface expression of biomarker (e.g., PD-L1) can be detected (e.g., using anti-human PD-L1 APC-conjugated antibody) and measured by ELISA or FACS. Also see Example 1 for exemplary method.

Bioactivities of binding domains (e.g., cytokine moieties) or immunomodulatory molecules described herein can also be reflected by in vivo or ex vivo experiments, for example, by measuring the proliferation of indicator cells (e.g., after administering IL-2 moieties or IL-2 immunomodulatory molecules, the proliferation of CD8+ cells, NK cells, or Tregs); by measuring the induction or inhibition of cytokine secretion; by measuring tumor volume reduction in tumor xenograft mice after injecting the test cytokine moieties or immunomodulatory molecules described herein; or by measuring autoimmune score.

Cell signaling assays can also be used to test bioactivities of binding domains (e.g., cytokine moieties) or immunomodulatory molecules described herein. Various cell signaling assay kits are commercially available, for example, to detect analytes produced during enzymatic reactions involved in signaling such as ADP, AMP, UDP, GDP, and growth factors, or phosphatase assays, to quantify both total and phosphorylated forms of signaling proteins. For example, after incubating the cells with cytokine moieties or immunomodulatory molecules described herein, to determine whether a particular kinase is active, the cell lysate is exposed to a known substrate for the enzyme in the presence of radioactive phosphate. The products are separated by electrophoresis (with or without immunoprecipitation), then the gel is exposed to x-ray film to determine whether the proteins incorporated the isotope. In some embodiments, the bioactivities of binding domains (e.g., cytokine moieties) or immunomodulatory molecules described herein on cells are measured by immunohistochemistry to locate signaling proteins. For example, antibodies to the signal proteins themselves or signal proteins in their activated state can be used. These antibodies have recognition epitopes that include the phosphate or other activating conformation. In some embodiments, movement of specific signaling proteins (e.g., nuclear translocation of signaling molecules) can be tracked by incorporating a fluorescent protein gene, e.g., green fluorescent protein (GFP), into genetic vectors encoding the protein to be studied. In some embodiments, bioactivities of binding domains (e.g., cytokine moieties) or immunomodulatory molecules described herein on cells are tested by western blots. For example, all tyrosine-phosphorylated proteins (or other phosphorylated amino acids, e.g., serine or threonine) can be detected with an anti-phosphotyrosine antibody (or antibodies against other phosphorylated amino acids) on a Western blot of cell lysates obtained after stimulation in a temporal sequence. In some embodiments, the bioactivities of binding domains (e.g., cytokine moieties) or immunomodulatory molecules described herein on cells can be measured by immunoprecipitation. For example, primary antibodies to a specific signaling protein or all tyrosine-phosphorylated proteins are cross-linked to the beads. The cells after incubating with cytokine moieties or immunomodulatory molecules described herein are lysed in buffer containing protease inhibitors and then incubated with the antibody-coated beads. The proteins are separated by using SDS electrophoresis, and then the proteins are identified by using the procedures described for Western blots. In some embodiments, glutathione S-transferase (GST) binding, or “pull-down” assay, can also be used, which determines direct protein-protein (e.g., signaling protein) interactions. Cell-based signal transduction assays can also be used. Briefly, a reporter cell line (e.g., HEK-Blue™) stably expressing the corresponding receptor of the test cytokine moiety or immunomodulatory molecule, corresponding signaling factors of the cytokine signaling pathway (e.g., STAT, JAK), and cytokine signaling pathway-inducible reporter (e.g., fluorescent protein, or secreted embryonic alkaline phosphatase) can be cultured in the presence of the test cytokine moiety or immunomodulatory molecule at 37° C. in a CO₂ incubator for sufficient time (e.g., 24-48 hours), then the reporter can be detected, such as using microscopy or FACS for fluorescent protein, or to detect secreted embryonic alkaline phosphatase in cell culture medium using colorimetric enzyme assay for alkaline phosphatase activity (e.g., QUANTI-Blue™).

Using IL-2 as an example of the first binding domain, STAT5 and ERK1/2 signaling can be measured to reflect IL-2 moiety or immunomodulatory molecule bioactivity, for example, by measuring phosphorylation of STAT5 and ERK1/2 using any suitable method known in the art. For example, STAT5 and ERK1/2 phosphorylation can be measured using antibodies specific for the phosphorylated version of these molecules in combination with flow cytometry analysis. For example, freshly isolated PBMCs are incubated at 37° C. with IL-2 or variant thereof, or IL-2 immunomodulatory molecule. After incubation, cells are immediately fixed (e.g., with Cytofix buffer) to preserve the phosphorylation status and permeabilized (e.g., with Phosflow Perm buffer III). The cells are stained with fluorophore-labeled antibodies against phosphorylated STAT5 or ERK1/2, and analyzed by flow cytometry. Alternatively, test samples (e.g., IL-2 cytokine moieties or IL-2 immunomodulatory molecules described herein) can be injected i.p. into mice, then total splenocytes can be isolated, immediately fixed (e.g., Phosphoflow™ Lyse/Fix buffer), washed with ice cold PBS, stained using anti-CD4 and anti-CD25 antibodies, and then permeabilized (e.g., PhosFlow Perm Buffer III). Cells are then washed with ice-cold FACS buffer, stained with anti-FoxP3, washed with ice-cold FACS buffer, and stained with fluorophore-labeled anti-phospho-STAT5 at room temperature. Cells are washed with FACS buffer, then data can be acquired on a FACS cytometer and analyzed. PI 3-kinase signaling can be measured using any suitable method known in the art to reflect IL-2 bioactivity, too. For example, PI 3-kinase signaling can be measured using antibodies that are specific for phospho-S6 ribosomal protein in conjunction with flow cytometry analysis.

In some embodiments, the first binding domain (e.g., immunostimulatory cytokine moieties) or immunomodulatory molecules described herein is capable of activating an immune cell, such as inducing test cytokine (e.g., IL-2 moiety or IL-2 immunomodulatory molecule described herein) dependent immune cell (e.g., PBMC, NK cell, CD8+ T cell, Th17 cell) proliferation, differentiation, and/or activation, cytokine secretion, activating signaling transduction (e.g., inducing STAT5 phosphorylation, ERK1/2 phosphorylation, or stimulating PI 3-kinase signaling), and/or inducing immune cells to kill tumor cells or infected cells. In some embodiments, the second binding domain (e.g., immunosuppressive cytokine moieties) or immunomodulatory molecules described herein is capable of inhibiting an immune cell, such as inhibiting cytokine (e.g., pro-inflammatory cytokine) production, antigen presentation, or MHC molecule expression from the immune cell, or inhibiting or ameliorating signaling transduction. In some embodiments, the immune cell is selected from the group consisting of a monocyte, a dendritic cell, a macrophage, a B cell, a killer T cell (T_{c}, cytotoxic T lymphocyte, or CTL), a helper T cell (Th), a regulatory T cells (Treg), a γδ T cell, a natural killer T (NKT) cell, and a natural killer (NK) cell.

In some embodiments, the activity in activating/inhibiting (or up-regulating/down-regulating) an immune response of the variant binding domain (e.g., cytokine variant) in a free state is the same or similar (such as within about ±20% difference) of that of a corresponding wildtype or non-variant binding domain (e.g., wildtype cytokine) in a free state. In some embodiments, the variant binding domain (e.g., cytokine variant) comprises a mutation or a modification (e.g., post-translational modification), which reduces its activity in activating/inhibiting (or up-regulating/down-regulating) an immune response compared to the wildtype or non-variant binding domain (e.g., wildtype cytokine) (e.g., no more than about any of 80%, 70%, 60%, 50%, 40%, 30%, 20%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0% of the bioactivity of wildtype or non-variant binding domain (e.g., wildtype cytokine)), when in a free state or in the absence of second target molecule-second binding domain binding of the immunomodulatory molecule described herein. In some embodiments, in the presence of second target molecule-second binding domain binding of the immunomodulatory molecule described herein, the activity in activating/inhibiting (or up-regulating/down-regulating) an immune response of the variant binding domain (e.g., cytokine variant) is at least about 1% (such as at least about any of 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, or 200%) of that of a corresponding wildtype or non-variant binding domain (e.g., wildtype cytokine).

### Hinge

Hinge connects the Fd region (V_{H} and CH1 domains) and the Fc region of a heavy chain of an immunoglobulin. In some embodiments, a hinge region connects a binding domain (e.g., ligand, receptor, VHH, scFv, or Fab) and an Fc domain subunit or portion thereof (e.g., CH2+CH3, or CH2 only). The hinge region, found in IgG, IgA, and IgD immunoglobulin classes, acts as a flexible spacer that allows the Fab portion of an immunoglobulin to move freely in space relative to the Fc region. The hinge domains are structurally diverse, varying in both sequence and length among immunoglobulin classes and subclasses. The heavy chains are inter-connected via disulfide bonds in the hinge region. According to crystallographic studies, the immunoglobulin hinge region can be further subdivided structurally and functionally into three regions: the upper hinge, the core, and the lower hinge. See Shin et al., Immunological Reviews 130:87 (1992). The upper hinge includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges. The lower hinge region joins the amino terminal end of, and includes residues in, the CH2 domain. Id. The hinge region of a human IgG1 antibody corresponds to amino acids 216-230 according to the EU numbering as set forth in Kabat. The core hinge region of human IgG₁ contains the sequence Cys-Pro-Pro-Cys that, when dimerized by disulfide bond formation, results in a cyclic octapeptide believed to act as a pivot, thus conferring flexibility. Conformational changes permitted by the structure and flexibility of the immunoglobulin hinge region polypeptide sequence may affect the effector functions of the Fc portion of the antibody.

In some embodiments, the hinge region may contain one or more glycosylation site(s), which include a number of structurally distinct types of sites for carbohydrate attachment. For example, IgA₁ contains five glycosylation sites within a 17 amino acid segment of the hinge region, conferring exceptional resistance of the hinge region polypeptide to intestinal proteases, considered an advantageous property for a secretory immunoglobulin.

In some embodiments, the immunomodulatory molecule comprises a hinge region that is present in a naturally occurring parental antibody. For example, the parental antibody is an IgG1 antibody, and the hinge region of the antibody or antigen-binding fragment within the immunomodulatory molecule described herein is an IgG1-type hinge region. In some embodiments, the immunomodulatory molecule contains a modification of the antibody heavy chain hinge region. For example, the hinge region or a portion thereof has been modified, e.g., by deletion, insertion, or replacement, e.g., with a hinge region or a portion thereof which differs from the hinge region present in a naturally occurring antibody of the same class (e.g., IgG, IgA, or IgE) and subclass (e.g., IgG₁, IgG₂, IgG₃, and IgG₄, etc.). For example, an IgG1, IgG2, or IgG3 antibody may contain an IgG4-type hinge region. In some embodiments, the hinge region or a portion thereof comprises a mutation, e.g., deletion, insertion, or replacement, at one or more of the upper hinge, the core, and the lower hinge of the hinge region, as long as inter-chain disulfide bond(s) can still be formed, the immunomodulatory molecule has flexibility to ensure target antigen-antigen binding fragment binding, masking cytokine activity in the absence of target antigen-antibody binding, while unmasking cytokine activity in the presence of target antigen-antibody binding, providing flexibility and/or sufficient space between two cytokine subunits or two cytokine moieties to ensure proper cytokine activity (binding affinity and/or bioactivity), and/or optionally does not abolish effector function(s) of the Fc portion. In some embodiments, the hinge region is or is derived from a human IgG1, IgG2, IgG3, or IgG4 hinge. In some embodiments, the hinge region is a mutated human IgG1, IgG2, IgG3, or IgG4 hinge. In some embodiments, one or more mutations, e.g., deletion, insertion, or replacement, are introduced at one or more of the upper hinge, the core, and the lower hinge of the hinge region in order to reduce or eliminate effector function (e.g., ADCC, and/or CDC) of the Fc domain, such as L234 and/or L235 mutations in the IgG1 lower hinge region, e.g., one or two of L234A, L234K, L234D, L235E, L235K, and L235A mutations. In some embodiments, the hinge region comprises L234K and L235K mutations. In some embodiments, the hinge region comprises L234D and L235E mutations. In some embodiments, the hinge region is truncated or mutated with less cysteines in order to reduce disulfide bond mis-pairing during dimerization of the Fc domain. In some embodiments, one or more asymmetric charged mutation(s) is introduced into the lower hinge to facilitate heterodimer formation, e.g., one polypeptide comprises L234K+L235K in the IgG1 lower hinge region, while the pairing polypeptide comprises L234D+L235E in the IgG1 lower hinge region. In some embodiments, the hinge region comprises the amino acid sequence of EPKSCDKTHTCPPCPAPELLGGP (SEQ ID NO: 76). In some embodiments, the hinge region is an IgG1 hinge comprising L234K and L235K mutations. In some embodiments, the hinge region comprises the amino acid sequence of EPKSCDKTHTCPPCPAPEKKGGP (SEQ ID NO: 77). In some embodiments, the hinge region is an IgG1 hinge comprising L234D and L235E mutations. In some embodiments, the hinge region comprises the amino acid sequence of EPKSCDKTHTCPPCPAPEDEGGP (SEQ ID NO: 78). In some embodiments, the hinge region comprises the amino acid sequence of ERKCCVECPPCPAPPVAGP (SEQ ID NO: 82). In some embodiments, the hinge region comprises the amino acid sequence of ESKYGPPCPSCPAPEFLGGP (SEQ ID NO: 83). In some embodiments, the hinge region comprises the amino acid sequence of ESKYGPPCPPCPAPEFLGGP (SEQ ID NO: 94). In some embodiments, the hinge region comprises the amino acid sequence of any of EPKSCDKDKTHTCPPCPAPELLGGP (SEQ ID NO: 79), EPKSCDKDKTHTCPPCPAPEKKGGP (SEQ ID NO: 80), or EPKSCDKDKTHTCPPCPAPEDEGGP (SEQ ID NO: 81). In some embodiments, the hinge region comprises the amino acid sequence of any of EPKSCDKPDKTHTCPPCPAPELLGGP (SEQ ID NO: 91), EPKSCDKPDKTHTCPPCPAPEKKGGP (SEQ ID NO: 92), EPKSCDKPDKTHTCPPCPAPEDEGGP (SEQ ID NO: 93), or EPPKSCDKTHTCPPCPAPELLGGP (SEQ ID NO: 95). In some embodiments, the hinge region, such as the hinge N′ portion, comprises the amino acid sequence of any of EPKSCDKP (SEQ ID NO: 90), EPKSCDK (SEQ ID NO: 84), or EPKSC (SEQ ID NO: 85). In some embodiments, the hinge region comprises the amino acid sequence of DKTHT (SEQ ID NO: 89). In some embodiments, the hinge region, such as the hinge C′ portion, comprises the amino acid sequence of any of DKTHTCPPCPAPELLGGP (SEQ ID NO: 86), DKTHTCPPCPAPEKKGGP (SEQ ID NO: 87), or DKTHTCPPCPAPEDEGGP (SEQ ID NO: 88). In some embodiments, the hinge comprises the sequence of any of SEQ ID NO: 76-95.

In some embodiments, the first binding domain (e.g., cytokine or variant thereof) described herein is positioned at the N-terminus of the hinge region of a heavy chain of a full-length antibody comprising the second binding domain, i.e., positioned between the C-terminus of the CH1 and the N-terminus of the hinge region of the heavy chain of the full-length antibody. In some embodiments, the heavy chain fusion polypeptide comprises from N′ to C′: VH-CH1—first binding domain (e.g., cytokine moiety)-hinge-CH2-CH3. In some embodiments, the first binding domain (e.g., cytokine or variant thereof) is positioned at the N-terminus of the hinge region between a second binding domain (e.g., ligand, receptor, VHH, scFv, or Fab) and an Fc domain subunit or portion thereof (e.g., CH2-CH3, or CH2). For example, in some embodiments, the immunomodulatory molecule comprises a polypeptide of any of from N′ to C′: (1) VH-first binding domain (e.g., cytokine moiety)-hinge-CH2-CH3; (2) VL-first binding domain (e.g., cytokine moiety)-hinge-CH2-CH3; (3) VH-optional linker-VL-first binding domain (e.g., cytokine moiety)-hinge-CH2-CH3; (4) VL-optional linker-VH-first binding domain (e.g., cytokine moiety)-hinge-CH2-CH3; (5) VH-CH1-first binding domain (e.g., cytokine moiety)-hinge-CH2-CH3; (6) VH-first binding domain (e.g., cytokine moiety)-hinge-CH2; (7) VL-cytokine moiety-hinge-CH2; (8) VH-optional linker-VL-first binding domain (e.g., cytokine moiety)-hinge-CH2; (9) VL-optional linker-VH-first binding domain (e.g., cytokine moiety)-hinge-CH2; (10) VH-CH1-first binding domain (e.g., cytokine moiety)-hinge-CH2; (11) ligand-optional linker-first binding domain (e.g., cytokine moiety)-hinge-CH2-CH3; (12) ligand-optional linker-first binding domain (e.g., cytokine moiety)-hinge-CH2; (13) receptor-optional linker-first binding domain (e.g., cytokine moiety)-hinge-CH2-CH3; or (14) receptor-optional linker-first binding domain (e.g., cytokine moiety)-hinge-CH2.

In some embodiments, the first binding domain (e.g., cytokine or variant thereof) described herein is positioned at the C-terminus of the hinge region of a heavy chain of a full-length antibody comprising the second binding domain, i.e., the heavy chain fusion polypeptide comprises from N′ to C′: VH-CH1-hinge-first binding domain (e.g., cytokine moiety)-CH2-CH3. In some embodiments, the first binding domain (e.g., cytokine or variant thereof) is positioned at the C-terminus of the hinge region between a second binding domain (e.g., ligand, receptor, VHH, scFv, or Fab) and an Fc domain subunit or portion thereof (e.g., CH2). For example, in some embodiments, the immunomodulatory molecule comprises a polypeptide of any of from N′ to C′: (1) VH-hinge-first binding domain (e.g., cytokine moiety)-CH2-CH3; (2) VL-hinge-first binding domain (e.g., cytokine moiety)-CH2-CH3; (3) VH-optional linker-VL-hinge-first binding domain (e.g., cytokine moiety)-CH2-CH3; (4) VL-optional linker-VH-hinge-first binding domain (e.g., cytokine moiety)-CH2-CH3; (5) VH-CH1-hinge-first binding domain (e.g., cytokine moiety)-CH2-CH3; (6) VH-hinge-first binding domain (e.g., cytokine moiety)-CH2; (7) VL-hinge-first binding domain (e.g., cytokine moiety)-CH2; (8) VH-optional linker-VL-hinge-first binding domain (e.g., cytokine moiety)-CH2; (9) VL-optional linker-VH-hinge-first binding domain (e.g., cytokine moiety)-CH2; (10) VH-CH1-hinge-first binding domain (e.g., cytokine moiety)-CH2; (11) ligand-hinge-first binding domain (e.g., cytokine moiety)-CH2-CH3; (12) ligand-hinge-first binding domain (e.g., cytokine moiety)-CH2; (13) receptor-hinge-first binding domain (e.g., cytokine moiety)-CH2-CH3; or (14) receptor-hinge-first binding domain (e.g., cytokine moiety)-CH2.

In some embodiments, the first binding domain (e.g., cytokine or variant thereof) described herein is positioned within the hinge region of a heavy chain of a full-length antibody comprising the second binding domain, i.e., the heavy chain fusion polypeptide comprises from N′ to C′: VH-CH1-hinge N′ portion-first binding domain (e.g., cytokine moiety)-hinge C′ portion-CH2-CH3. In some embodiments, the cytokine or variant thereof replaces a portion of the hinge region. In some embodiments, the cytokine or variant thereof is inserted within the hinge region, without deleting any hinge amino acid. In some embodiments, the cytokine or variant thereof with a peptide linker fused to the N′ of the cytokine or variant thereof is inserted within the hinge region. In some embodiments, the cytokine or variant thereof with a peptide linker fused to the C′ of the cytokine or variant thereof is inserted within the hinge region. For example, in some embodiments, the hinge-cytokine portion comprises a structure of from N′ to C′: hinge N′ portion-optional N′ peptide linker-first binding domain (e.g., cytokine moiety)-optional C′ peptide linker-hinge C′ portion. In some embodiments, the hinge region is an IgG1 hinge, and the cytokine or variant thereof is inserted between “EPKSC” (SEQ ID NO: 85) and “DKTHT” (SEQ ID NO: 89). In some embodiments, the N′ peptide linker comprises the amino acid sequence of DKP (SEQ ID NO: 231) or P (SEQ ID NO: 242). Hence in some embodiments, the cytokine or variant thereof is inserted between an additionally introduced “DKP” and the “DKTHT” sequence. In some embodiments, the N′ peptide linker comprises the amino acid sequence of DKPGS (SEQ ID NO: 232), PGS (SEQ ID NO: 233), or GS (SEQ ID NO: 234). In some embodiments, the N′ peptide linker comprises the amino acid sequence of DKPGSG (SEQ ID NO: 235), PGSG (SEQ ID NO: 236), or GSG (SEQ ID NO: 203). In some embodiments, the N′ peptide linker comprises the amino acid sequence of DKPGSGS (SEQ ID NO: 237), PGSGS (SEQ ID NO: 238), or GSGS (SEQ ID NO: 239). In some embodiments, the N′ peptide linker comprises the amino acid sequence of DKPGSGGGGG (SEQ ID NO: 240), PGSGGGGG (SEQ ID NO: 241), GSGGGGG (SEQ ID NO: 206).In some embodiments, the cytokine or variant thereof is positioned within the hinge region between an antigen-binding fragment (e.g., ligand, receptor, VHH, scFv, or Fab) and an Fc domain subunit or portion thereof (e.g., CH2). For example, in some embodiments, the immunomodulatory molecule comprises a polypeptide of any of from N′ to C′: (1) VH-hinge N′ portion-optional N′ peptide linker-first binding domain (e.g., cytokine moiety)-optional C′ peptide linker-hinge C′ portion-CH2-CH3; (2) VL-hinge N′ portion-optional N′ peptide linker-first binding domain (e.g., cytokine moiety)-optional C′ peptide linker-hinge C′ portion-CH2-CH3; (3) VH-optional linker-VL-hinge N′ portion-optional N′ peptide linker-first binding domain (e.g., cytokine moiety)-optional C′ peptide linker-hinge C′ portion-CH2-CH3; (4) VL-optional linker-VH-hinge N′ portion-optional N′ peptide linker-first binding domain (e.g., cytokine moiety)-optional C′ peptide linker-hinge C′ portion-CH2-CH3; (5) VH-CH1-hinge N′ portion-optional N′ peptide linker-first binding domain (e.g., cytokine moiety)-optional C′ peptide linker-hinge C′ portion-CH2-CH3; (6) VH-hinge N′ portion-optional N′ peptide linker-first binding domain (e.g., cytokine moiety)-optional C′ peptide linker-hinge C′ portion-CH2; (7) VL-hinge N′ portion-optional N′ peptide linker-first binding domain (e.g., cytokine moiety)-optional C′ peptide linker-hinge C′ portion-CH2; (8) VH-optional linker-VL-hinge N′ portion-optional N′ peptide linker-first binding domain (e.g., cytokine moiety)-optional C′ peptide linker-hinge C′ portion-CH2; (9) VL-optional linker-VH-hinge N′ portion-optional N′ peptide linker-first binding domain (e.g., cytokine moiety)-optional C′ peptide linker-hinge C′ portion-CH2; (10) VH-CH1-hinge N′ portion-optional N′ peptide linker-first binding domain (e.g., cytokine moiety)-optional C′ peptide linker-hinge C′ portion-CH2; (11) ligand-optional linker-hinge N′ portion-optional N′ peptide linker-first binding domain (e.g., cytokine moiety)-optional C′ peptide linker-hinge C′ portion-CH2-CH3; (12) ligand-optional linker-hinge N′ portion-optional N′ peptide linker-first binding domain (e.g., cytokine moiety)-optional C′ peptide linker-hinge C′ portion-CH2; (13) receptor-optional linker-hinge N′ portion-optional N′ peptide linker-first binding domain (e.g., cytokine moiety)-optional C′ peptide linker-hinge C′ portion-CH2-CH3; or (14) receptor-optional linker-hinge N′ portion-optional N′ peptide linker-first binding domain (e.g., cytokine moiety)-optional C′ peptide linker-hinge C′ portion-CH2.

### Fc Domains

In some embodiments, the immunomodulatory molecule descried herein comprises an Fc domain or portion thereof. Fc domain comprises a CH2 domain and a CH3 domain. In some embodiments, the Fc domain portion comprises (consists essentially of or consists of) a CH2 domain. In some embodiments, the Fc domain portion comprises (consists essentially of or consists of) a CH3 domain.

In some embodiments, the Fc domain is derived from any of IgA, IgD, IgE, IgG, and IgM, and subtypes thereof. In some embodiments, the Fc domain comprises CH2 and CH3. In some embodiments, the Fc domain is derived from an IgG (e.g., IgG1, IgG2, IgG3, or IgG4). In some embodiments, the Fc domain is derived from a human IgG. In some embodiments, the Fc domain is derived from a human IgG1 or human IgG4. In some embodiments, the two subunits of the Fc domain dimerize via one or more (e.g., 1, 2, 3, 4, or more) disulfide bonds. In some embodiments, each subunit of the Fc domain comprises a full-length Fc sequence. In some embodiments, each subunit of the Fc domain comprises an N-terminus truncated Fc sequence. In some embodiments, the Fc domain is truncated at the N-terminus, e.g., lacks the first 1, 2, 3, 4, 5, 6, 7, 8, 9, or 10 amino acids of a complete immunoglobulin Fc domain. In some embodiments, the Fc domain comprises the amino acid sequence of any of SEQ ID NOs: 96-102.

Via the Fc domain, immunomodulatory molecules can activate complement and interact with Fc receptors. This inherent immunoglobulin feature has been viewed unfavorably because immunomodulatory molecules may be targeted to cells expressing Fc receptors rather than the preferred antigen-bearing cells. Moreover, the simultaneous activation of cytokine receptors and Fc receptor signaling pathways leading to cytokine release, especially in combination with the long half-life of immunoglobulin fusion proteins, make their application in a therapeutic setting difficult due to systemic toxicity. Thus in some embodiments, the Fc domain is engineered to have altered binding to an Fc receptor (FcR), specifically altered binding to an Fcγ receptor, and/or altered effector function, such as altered (e.g., reduced or eliminated) antibody-dependent cell-mediated cytotoxicity (ADCC), Antibody-dependent Cellular Phagocytosis (ADCP), and/or Complement-dependent cytotoxicity (CDC).

Although the presence of an Fc domain is essential for prolonging the half-life of the immunomodulatory molecule, in some situations it will be beneficial to eliminate effector functions associated with engagement of Fc receptors by the Fc domain. Hence, in some embodiments the altered binding to an Fc receptor and/or effector function is reduced binding and/or effector function. In some embodiments, the Fc domain comprises one or more amino acid mutation that reduces the binding of the Fc domain to an Fc receptor, particularly an Fcγ receptor (responsible for ADCC). Preferably, such an amino acid mutation does not reduce binding to FcRn receptors (responsible for half-life). In some embodiments, the Fc domain is derived from human IgG1 and comprises the amino acid substitution N295A. In some embodiments, the Fc domain is derived from human IgG4 and comprises the amino acid substitutions S228P and L235E at the hinge region. In some embodiments, the Fc domain is derived from human IgG1 and comprises the amino acid substitutions L234A and L235A (“LALA”) at the hinge region. In some embodiments, the Fc domain is derived from human IgG1 and comprises the amino acid substitutions L234A and L235A at the hinge region, and P329G, e.g., in each of its subunits. See, e.g., Lo M. et al. J Biol Chem. 2017 Mar. 3; 292(9):3900-3908. Schlothauer T. et al. Protein Eng Des Sel. 2016 October; 29(10):457-466.

In some embodiments, the Fc domain (e.g., human IgG1) is mutated to remove one or more effector functions such as ADCC, ADCP, or CDC, namely, an “effectorless” or “almost effectorless” Fc domain. For example, in some embodiments, the Fc domain is an effectorless IgG1 Fc comprising one or more of the following mutations (such as in each of its subunits): L234A, L235E, G237A, A330S, and P331S. The combinations of K322A, L234A, and L235A in IgG1 are sufficient to almost completely abolish FcγR and C1q binding (Hezareh et al. J Virol 75, 12161-12168, 2001). MedImmune identified that a set of three mutations L234F/L235E/P331S have a very similar effect (Oganesyan et al., Acta Crystallographica 64, 700-704, 2008). In some embodiments, the Fc domain comprises a modification of the glycosylation on N297 of the IgG1 Fc domain, which is known to be required for optimal FcR interaction. The Fc domain modification can be any suitable IgG Fc engineering mentioned in Wang et al. (“IgG Fc engineering to modulate antibody effector functions,” Protein Cell. 2018 January; 9(1): 63-73), the content of which is incorporated herein by reference in its entirety.

In some embodiments, the Fc domain comprises two identical polypeptide chains (identical Fc subunits). Such Fc domains are herein also referred to as “homodimeric Fc domains.” In some embodiments, each subunit of the homodimeric Fc domain comprises the amino acid sequence of any of SEQ ID NOs: 96, and 99-102.

In some embodiments, the Fc domain comprises a modification promoting heterodimerization of two non-identical polypeptide chains. Such Fc domains are herein also referred to as “heterodimeric Fc domains.” In some embodiments, the Fc domain comprises a knob-into-hole (KIH) modification, comprising a knob modification in one of the subunits of the Fc domain and a hole modification in the other one of the two subunits of the Fc domain. Any suitable knob-into-hole modifications can be applied to the immunomodulatory molecule described herein, such as amino acid changes of T22>Y (creating the knob) in strand B of the first CH3 domain and Y86>T (creating the hole) in strand E of the partner CH3 domain. Also see US20200087414, the content of which is incorporated herein by reference in its entirety. In some embodiments, one subunit of the Fc domain comprises one or more of T350V, L351Y, S400E, F405A, and Y407V mutations relative to a wildtype human IgG1 Fc, and the other subunit of the Fc domain comprises one or more of T350V, T366L, N390R, K392M, T394W mutations relative to a wildtype human IgG1 Fc. In some embodiments, one subunit of the Fc domain comprises the sequence of SEQ ID NO: 97, and the other subunit of the Fc domain comprises the sequence of SEQ ID NO: 98.

In some embodiments, the Fc domain is a single chain Fc domain as described in WO2017134140, the content of which is incorporated herein by reference in its entirety.

### Linkers

In some embodiments, within the immunomodulatory molecule described herein, between the two or more binding domains connected in tandem, the second binding domain (e.g., ligand, receptor, VHH, scFv, or Fab) and the first binding domain (e.g., cytokine moiety), the first binding domain (e.g., cytokine moiety) and CL, the first binding domain (e.g., cytokine moiety) and VH, the first binding domain (e.g., cytokine moiety) and VL, the CH1 domain and the first binding domain (e.g., cytokine moiety), the two or more first binding domains (e.g., cytokine moiety) connected in tandem, the two or more subunits of a cytokine or variant thereof connected in tandem, the first binding domain (e.g., cytokine moiety) and the Fc domain subunit or portion thereof, the hinge region and the CH1 domain, the hinge region and the CH2 domain, the hinge region and the first binding domain (e.g., cytokine moiety), the Fc domain subunit or portion thereof and the antigen-binding fragment, and/or the CH1 domain and the Fc domain subunit or portion thereof, are connected via one or more optional linkers (e.g., peptide linker, non-peptide linker). In some embodiments, the one or more linkers are the same. In some embodiments, the one or more linkers are different (e.g., different from each other). In some embodiments, the one or more linkers are flexible linkers. In some embodiments, the one or more linkers are stable linkers. In some embodiments, some of the linkers are flexible, while others are stable. In general, a linker does not affect or significantly affect the proper fold and conformation formed by the configuration of the immunomodulatory molecule. In some embodiments, the linker confers flexibility and spatial space for each portion of the immunomodulatory molecule, such as allows target antigen-antigen binding fragment binding, allows ligand-receptor binding, masking first binding domain (e.g., cytokine) activity in the absence of second target molecule-second binding domain binding, while unmasking first binding domain (e.g., cytokine) activity in the presence of second target molecule-second binding domain binding, providing flexibility and/or sufficient space between two binding domains or domain subunits (e.g., cytokine subunits or two cytokine moieties) to ensure proper binding domain (e.g., cytokine) activity (binding affinity and/or bioactivity), etc.

The linkers can be peptide linkers of any length. In some embodiments, the peptide linker is from about 1 amino acid (aa) to about 10 aa long, from about 2 aa to about 15 aa long, from about 3 aa to about 12 aa long, from about 4 aa to about 10 aa long, from about 5 aa to about 9 aa long, from about 6 aa to about 8 aa long, from about 1 amino acid to about 20 aa long, from about 21 aa to about 30 aa long, from about 1 amino acid to about 30 aa long, from about 2 aa to about 20 aa long, from about 10 aa to about 30 as long, from about 1 amino acid to about 50 aa long, from about 2 aa to about 19 aa long, from about 2 aa to about 18 aa long, from about 2 aa to about 17 aa long, from about 2 aa to about 16 as long, from about 2 aa to about 10 aa long, from about 2 aa to about 14 aa long, from about 2 aa to about 13 aa long, from about 2 aa to about 12 as long, from about 2 aa to about 11 aa long, from about 2 aa to about 9 aa long, from about 2 aa to about 8 aa long, from about 2 aa to about 7 aa long, from about 2 aa to about 6 aa long, from about 2 as to about 5 aa long, or from about 6 aa to about 30 aa long. In some embodiments, the peptide linker is about any of 1, 2, 3, 4, 5, 6, 7, 8, 9, 10, 11, 12, 13, 14, 15, 16, 17, 18, 19 or 20 amino acids long. In some embodiments, the peptide linker is about any of 21, 22, 23, 24, 25, 26, 27, 28, 29, or 30 amino acids long. In some embodiments, the peptide linker is about any of 31, 32, 33, 34, 35, 36, 37, 38, 39, 40, 41, 42, 43, 44, 45, 46, 47, 48, 49, or 50 amino acids long. In some embodiments, the linker is about 10 to about 20 amino acids in length.

A peptide linker can have a naturally occurring sequence or a non-naturally occurring sequence. For example, a sequence derived from the hinge region of a heavy chain only antibody can be used as a linker. See, for example, WO1996/34103. In some embodiments, the peptide linker is a human IgG1, IgG2, IgG3, or IgG4 hinge or portion thereof. In some embodiments, the peptide linker is a mutated human IgG1, IgG2, IgG3, or IgG4 hinge or portion thereof. In some embodiments, the linker is a flexible linker. Exemplary flexible linkers include, but are not limited to, glycine polymers (G)ₙ (SEQ ID NO: 194), glycine-serine polymers (including, for example, (GS)ₙ (SEQ ID NO: 195), (GGS)ₙ (SEQ ID NO: 196), (GGGS)ₙ (SEQ ID NO: 197), (GGS)ₙ(GGGS)ₙ (SEQ ID NO: 198), (GSGGS)ₙ (SEQ ID NO: 199), (GGSGS)ₙ (SEQ ID NO: 200), or (GGGGS)ₙ (SEQ ID NO: 201), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine and is much less restricted than residues with longer side chains (see Scheraga, Rev. Computational Chem. 11 173-142 (1992)). Exemplary flexible linkers include, but are not limited to GG (SEQ ID NO. 202), GSG (SEQ ID NO: 203), GGSG (SEQ ID NO: 204), GGSGG (SEQ ID NO: 205), GSGGGGG (SEQ ID NO: 206), GSGSG (SEQ ID NO: 207), GSGGG (SEQ ID NO. 208), GGGSG (SEQ ID NO: 209), GSSSG (SEQ ID NO: 210), GGSGGS (SEQ ID NO: 211), SGGGGS (SEQ ID NO: 212), GGGGS (SEQ ID NO: 213), (GA)ₙ (SEQ ID NO: 214, n is an integer of at least 1), GRAGGGGAGGGG (SEQ ID NO: 215), GRAGGG (SEQ ID NO: 216), GSGGGSGGGGSGGGGS (SEQ ID NO: 217), GGGSGGGGSGGGGS (SEQ ID NO: 218), GGGSGGSGGS (SEQ ID NO: 219), GGSGGSGGSGGSGGG (SEQ ID NO: 220), GGSGGSGGGGSGGGGS (SEQ ID NO: 221), GGSGGSGGSGGSGGSGGS (SEQ ID NO: 222), GGGGSGGGGSGGGGS (SEQ ID NO: 229), GGGGGGSGGGGSGGGGSA (SEQ ID NO: 223), GSGGGSGGGGSGGGGSGGGGS (SEQ ID NO: 224), KTGGGSGGGS (SEQ ID NO: 225), GGPGGGGSGGGSGGGGS (SEQ ID NO: 226), GGGSGGGGSGGGGSGGGGS (SEQ ID NO: 227), GGGGSGGGGSGGGGSGGGGSG (SEQ ID NO: 228), and the like. In some embodiments, the linker comprises the sequence of ASTKGP (SEQ ID NO: 230). In some embodiments, the linker comprises the sequence of any one of SEQ ID NOs: 194-246. The ordinarily skilled artisan will recognize that design of an immunomodulatory molecule can include linkers that are all or partially flexible, such that the linker can include a flexible linker portion as well as one or more portions that confer less flexible structure to provide a desired immunomodulatory molecule structure and function (e.g., masking cytokine activity in the absence of target antigen-antibody binding, while unmasking cytokine activity in the presence of target antigen-antibody binding; or providing flexibility and/or sufficient space between two cytokine subunits to ensure proper cytokine activity (binding affinity and/or bioactivity)). In some embodiments, the peptide linker is enriched in serine-glycine. In some embodiments, the cytokine moiety described herein comprises two cytokine subunits (wildtype or mutant) connected by a linker, such as a peptide linker comprising any of SEQ ID NOs: 227-229, 245, and 246.

In some embodiments, the linker is a stable linker (e.g., not cleavable by protease, especially MMPs).

Any one or all of the linkers described herein can be accomplished by any chemical reaction that will connect the two or more binding domains connected in tandem, between the second binding domain (e.g., ligand, receptor, VHH, scFv, or Fab) and the first binding domain (e.g., cytokine moiety), between the first binding domain (e.g., cytokine moiety) and CL, the first binding domain (e.g., cytokine moiety) and VH, the first binding domain (e.g., cytokine moiety) and VL, the CH1 domain and the first binding domain (e.g., cytokine moiety), the two or more first binding domains (e.g., cytokine moiety) connected in tandem, the two or more subunits of a cytokine or variant thereof connected in tandem, the first binding domain (e.g., cytokine moiety) and the Fc domain subunit or portion thereof, the hinge region and the CH1 domain, the hinge region and the CH2 domain, the hinge region and the first binding domain (e.g., cytokine moiety), the Fc domain subunit or portion thereof and the antigen-binding fragment, and/or the CH1 domain and the Fc domain subunit or portion thereof, so long as the components or fragments retain their respective activities, i.e. binding to cytokine receptor, binding to target antigen(s), binding to ligand or receptor, binding to FcR, or ADCC. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents are useful in coupling protein molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester)). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pyridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above can contain components that have different attributes, thus leading to immunomodulatory molecules with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form fusion protein with increased stability. Disulfide linkages, are in general, less stable than other linkages because the disulfide linkage is cleaved in vitro, resulting in less fusion protein available. Sulfo-NHS, in particular, can enhance the stability of carbodiimide couplings. Carbodiimide couplings (such as EDC) when used in conjunction with sulfo-NHS, forms esters that are more resistant to hydrolysis than the carbodiimide coupling reaction alone.

Other linker considerations include the effect on physical or pharmacokinetic properties of the resulting immunomodulatory molecule, such as solubility, lipophilicity, hydrophilicity, hydrophobicity, stability (more or less stable as well as planned degradation), rigidity, flexibility, immunogenicity, modulation of cytokine moiety/cytokine receptor binding, modulation of antigen-binding domain/target antigen binding, modulation of ligand-receptor binding, the ability to be incorporated into a micelle or liposome, and the like.

### Immunomodulatory Molecule Variants

### Glycosylation Variants

In some embodiments, the immunomodulatory molecule is altered to increase or decrease the extent to which the construct is glycosylated. Addition or deletion of glycosylation sites to an Fc domain may be conveniently accomplished by altering the amino acid sequence such that one or more glycosylation sites is created or removed.

Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an Fc domain may be made in order to create certain improved properties.

In some embodiments, the immunomodulatory molecule described herein is provided having a carbohydrate structure that lacks fucose attached (directly or indirectly) to the Fc domain. For example, the amount of fucose in such immunomodulatory molecule may be from 1% to 80%, from 1% to 65%, from 5% to 65% or from 20% to 40/c. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc domain (EU numbering of Fc region residues); however, Asn297 may also be located about f 3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

### Effector Function Variants

In some embodiments, the present application contemplates an immunomodulatory molecule that possesses some but not all Fc effector functions, which makes it a desirable candidate for applications in which the half-life of the immunomodulatory molecule in vivo is important yet certain effector functions (such as CDC and ADCC) are unnecessary or deleterious. Some of the Fc domain variants have been discussed above. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Erp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Fc domains with reduced effector function include those with substitution of one or more of Fc region residues 238, 265, 269, 270, 297, 327 and 329 (U.S. Pat. No. 6,737,056). Such Fc mutants include substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described (see, e.g., U.S. Pat. No. 6,737,056; WO 2004/056312, and Shields et al., J. Biol. Chem. 9(2): 6591-6604 (2001)). In some embodiments, alterations are made in the Fc domain that result in altered (i.e., either improved or diminished) C1q binding and/or CDC, e.g., as described in U.S. Pat. No. 6,194,551, WO 99/51642, and Idusogie et al. J. Immunol. 164: 4178-4184 (2000).

In some embodiments, the Fc domain comprises one or more amino acid substitutions, which increase half-life and/or improve binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc domain with one or more substitutions therein which improve binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc domain variants.

### Cysteine Engineered Variants

In some embodiments, it may be desirable to create cysteine-engineered immunomodulatory molecules, e.g., “thioMAbs,” in which one or more residues of an immunomodulatory molecule are substituted with cysteine residues. In particular, embodiments, the substituted residues occur at accessible sites of the immunomodulatory molecule. By substituting those residues with cysteine, reactive thiol groups are thereby positioned at accessible sites of the immunomodulatory molecule and may be used to conjugate the immunomodulatory molecule to other moieties, such as drug moieties or linker-drug moieties, to create an immunomodulatory molecule-conjugate. In some embodiments, any one or more of the following residues may be substituted with cysteine: Al 18 (EU numbering) of the heavy chain; and S400 (EU numbering) of the heavy chain Fc domain. Cysteine engineered immunomodulatory molecules may be generated as described, e.g., in U.S. Pat. No. 7,521,541.

### Immunomodulatory Molecule Derivatives

In some embodiments, immunomodulatory molecules provided herein may further comprise an additional therapeutic compound, such as any therapeutic compounds known in the art. For example, the parental antibody in some embodiments can be an antibody drug conjugate (ADC). See, e.g., any ADC described in Shim H. (Biomolecules. 2020 March; 10(3): 360), and Diamantis N. and Banerji U. Br J Cancer. 2016; 114(4): 362-367, the contents of which are incorporated herein by reference in their entirety. In some embodiments, the therapeutic compound is conjugated to the Fc domain or portion thereof. In some embodiments, the therapeutic compound is a cytotoxic agent, a chemotherapeutic agent, a growth inhibitory agent, or an antibiotic.

In some embodiments, the immunomodulatory molecule further comprises a label selected from the group consisting of a chromophore, a fluorophore (e.g., coumarin, a xanthene, a cyanine, a pyrene, a borapolyazaindacene, an oxazine, and derivatives thereof), a fluorescent protein (e.g., GFP, phycobiliproteins, and derivatives thereof), a phosphorescent dye (e.g., dioxetanes, xanthene, or carbocyanine dyes, lanthanide chelates), a tandem dye (e.g., cyanine-phycobiliprotein derivative and xanthene-phycobiliprotein derivative), a particle (e.g., gold clusters, colloidal gold, microspheres, quantum dots), a hapten, an enzyme (e.g., peroxidase, a phosphatase, a glycosidase, a luciferase), and a radioisotope (e.g., ¹²⁵I, ³H, ¹⁴C, ³²P).

### III. Vectors Encoding Immunomodulatory Molecules

The present invention also provides isolated nucleic acids encoding any of the immunomodulatory molecules described herein (such as described in any of FIGS. 1A-1W and 11A-15D, Examples, and Sequence Listing herein, e.g., IL-2/anti-PD-1 agonist Ab immunomodulatory molecule, IL-12/anti-PD-1 agonist Ab immunomodulatory molecule, IL-2/PD-L1 immunomodulatory molecule, IL-12/PD-L1 immunomodulatory molecule, IL-2/PD-L2 immunomodulatory molecule, IL-12/PD-L2 immunomodulatory molecule), vectors comprising nucleic acids encoding any of the immunomodulatory molecules described herein. Also provided are isolated host cells (e.g., CHO cells, HEK 293 cells, Hela cells, COS cells) comprising nucleic acids encoding any of the immunomodulatory molecules described herein, or vectors comprising nucleic acids encoding any of the immunomodulatory molecules described herein.

In some embodiments, the vector comprising a nucleic acid encoding any of the immunomodulatory molecules described herein is suitable for replication and integration in eukaryotic cells, such as mammalian cells (e.g., CHO cells, HEK 293 cells, Hela cells, COS cells). In some embodiments, the vector is a viral vector. Examples of viral vectors include, but are not limited to, adenoviral vectors, adeno-associated virus vectors, lentiviral vector, retroviral vectors, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulatory molecule coding sequence(s) can be packaged with protocols known in the art. The resulting lentiviral vectors can be used to transduce a mammalian cell using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector is a non-viral vector. In some embodiments, the vector is a transposon, such as a Sleeping Beauty (SB) transposon system, or a PiggyBac transposon system. In some embodiments, the vector is a polymer-based non-viral vector, including for example, poly (lactic-co-glycolic acid) (PLGA) and poly lactic acid (PLA), poly (ethylene imine) (PEI), and dendrimers. In some embodiments, the vector is a cationic-lipid based non-viral vector, such as cationic liposome, lipid nanoemulsion, and solid lipid nanoparticle (SLN). In some embodiments, the vector is a peptide-based gene non-viral vector, such as poly-L-lysine. Any of the known non-viral vectors suitable for genome editing can be used for introducing the immunomodulatory molecule-encoding nucleic acid(s) to the host cells. See, for example, Yin H. et al. Nature Rev. Genetics (2014) 15:521-555; Aronovich E L et al. “The Sleeping Beauty transposon system: a non-viral vector for gene therapy.” Hum. Mol. Genet. (2011) R1: R14-20; and Zhao S. et al. “PiggyBac transposon vectors: the tools of the human gene editing.” Transl. Lung Cancer Res. (2016) 5(1): 120-125, which are incorporated herein by reference. In some embodiments, any one or more of the nucleic acids or vectors encoding the immunomodulatory molecules described herein is introduced to the host cells (e.g., CHO, HEK 293, Hela, or COS) by a physical method, including, but not limited to electroporation, sonoporation, photoporation, magnetofection, hydroporation.

In some embodiments, the vector contains a selectable marker gene or a reporter gene to select cells expressing the immunomodulatory molecules described herein from the population of host cells transfected through vectors (e.g., lentiviral vectors). Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

In some embodiments, the vector (e.g., viral vector) comprises any one of the nucleic acids encoding the immunomodulatory molecules described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present invention. Promoters may be roughly categorized as constitutive promoters or regulated promoters, such as inducible promoters.

In some embodiments, the nucleic acid encoding the immunomodulatory molecules described herein is operably linked to a constitutive promoter. Constitutive promoters allow heterologous genes (also referred to as transgenes) to be expressed constitutively in the host cells. Exemplary promoters contemplated herein include, but are not limited to, cytomegalovirus immediate-early promoter (CMV), human elongation factors-1alpha (hEF1α), ubiquitin C promoter (UbiC), phosphoglycerokinase promoter (PGK), simian virus 40 early promoter (SV40), chicken β-Actin promoter coupled with CMV early enhancer (CAGG), a Rous Sarcoma Virus (RSV) promoter, a polyoma enhancer/herpes simplex thymidine kinase (MC1) promoter, a beta actin (β-ACT) promoter, a “myeloproliferative sarcoma virus enhancer, negative control region deleted, d1587rev primer-binding site substituted (MND)” promoter. The efficiencies of such constitutive promoters on driving transgene expression have been widely compared in a huge number of studies. In some embodiments, the nucleic acid encoding the immunomodulatory molecules described herein is operably linked to a CMV promoter.

In some embodiments, the nucleic acid encoding the immunomodulatory molecules described herein is operably linked to an inducible promoter. Inducible promoters belong to the category of regulated promoters. The inducible promoter can be induced by one or more conditions, such as a physical condition, microenvironment of the host cells, or the physiological state of the host cells, an inducer (i.e., an inducing agent), or a combination thereof. In some embodiments, the inducing condition does not induce the expression of endogenous genes in the host cell. In some embodiments, the inducing condition is selected from the group consisting of: inducer, irradiation (such as ionizing radiation, light), temperature (such as heat), redox state, and the activation state of the host cell. In some embodiments, the inducible promoter can be an NFAT promoter, a TETON® promoter, or an NFκB promoter. In some embodiments, the inducible promoter is a tet-inducible promoter.

In some embodiments, the vector comprises more than one nucleic acids encoding the immunomodulatory molecules described herein, e.g., different polypeptides of the immunomodulatory molecule. In some embodiments, each vector comprises 2 nucleic acids encoding 2 polypeptides of the immunomodulatory molecules described herein.

In some embodiments, the two or more nucleic acids encoding the immunomodulatory molecules described herein are operably regulated under the same promoter in the vector. In some embodiments, the two or more nucleic acids are linked in tandem via a linking sequence (e.g., IRES) or a nucleic acid sequence encoding a self-cleaving 2A peptide, such as P2A, T2A, E2A, F2A, BmCPV 2A, BmIFV 2A. In some embodiments, the nucleic acid encoding two or more polypeptides of the immunomodulatory molecules comprises linking sequence(s) (e.g., IRES) or nucleic acid sequence(s) encoding self-cleaving 2A peptide(s) (such as P2A, T2A, E2A, F2A, BmCPV 2A, BmIFV 2A) between the polypeptide encoding sequences. In some embodiments, the two or more nucleic acids encoding the immunomodulatory molecules described herein are operably regulated under separate promoters in the vector. In some embodiments, the promoters operably linked to each nucleic acid are different. In some embodiments, the promoters operably linked to each nucleic acid are the same. In some embodiments, the immunomodulatory molecule described herein is encoded by two or more vectors, e.g., each vector encodes one heavy chain (or one polypeptide comprising VH and cytokine moiety) and one pairing light chain, or each vector encodes one polypeptide of the immunomodulatory molecule.

### IV. Methods of Preparation

Also provided are methods of preparing any of the immunomodulatory molecules described herein (such as described in any of FIGS. 1A-1W and 11A-15D, Examples, and Sequence Listing herein, e.g., IL-2/anti-PD-1 agonist Ab immunomodulatory molecule, IL-12/anti-PD-1 agonist Ab immunomodulatory molecule, IL-2/PD-L1 immunomodulatory molecule, IL-12/PD-L1 immunomodulatory molecule, IL-2/PD-L2 immunomodulatory molecule, IL-12/PD-L2 immunomodulatory molecule). Thus, in some embodiments, there is provided a method of producing an immunomodulatory molecule, comprising: (a) culturing a host cell (e.g., CHO cell, HEK 293 cell, Hela cell, or COS cell) comprising any of the nucleic acids or vectors encoding the immunomodulatory molecules described herein under a condition effective to express the encoded immunomodulatory molecule; and (b) obtaining the expressed immunomodulatory molecule from said host cell. In some embodiments, the method of step (a) further comprises producing a host cell comprising the nucleic acid or vector encoding the immunomodulatory molecule described herein. The immunomodulatory molecule described herein may be prepared using any methods known in the art or as described herein. Also see Examples 1, 4, 5, 7, 9, 10, and 12 for exemplary methods. In some embodiments, the immunomodulatory molecules is expressed with eukaryotic cells, such as mammalian cells. In some embodiments, the immunomodulatory molecules is expressed with prokaryotic cells.

### 1. Recombinant Production in Prokaryotic Cells

### a) Vector Construction

Polynucleic acid sequences encoding the immunomodulatory molecules of the present application can be obtained using standard recombinant techniques. Desired polynucleic acid sequences may be isolated and sequenced from antibody or immunomodulatory molecule producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector, which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g., the presence or absence of a nutrient or a change in temperature.

A large number of promoters recognized by a variety of potential host cells are well known. The selected promoter can be operably linked to cistron DNA encoding the polypeptide by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the—galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target light and heavy chains (Siebenlist et al. (1980) Cell 20: 269) using linkers or adaptors to supply any required restriction sites.

In some embodiments, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e., cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STH) leaders, LamB, PhoE, PelB, OmpA and MBP. In some embodiments of the present application, the signal sequences used in both cistrons of the expression system are STII signal sequences or variants thereof.

In some embodiments, the production of the immunomodulatory molecule according to the present application can occur in the cytoplasm of the host cell, and therefore does not require the presence of secretion signal sequences within each cistron. In some embodiments, polypeptide components are expressed, folded, and assembled to form an immunomodulatory molecule (or portion of the immunomodulatory molecule) within the cytoplasm. Certain host strains (e.g., the E. coli trxB⁻ strains) provide cytoplasm conditions that are favorable for disulfide bond formation, thereby permitting proper folding and assembly of expressed protein subunits. See Proba and Pluckthun, Gene, 159:203 (1995).

The present invention provides an expression system in which the quantitative ratio of expressed polypeptide components can be modulated in order to maximize the yield of secreted and properly assembled immunomodulatory molecules of the present application. Such modulation is accomplished at least in part by simultaneously modulating translational strengths for the polypeptide components. One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleic acid sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired protein products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the present application.

### b) Prokaryotic Host Cells

Prokaryotic host cells suitable for expressing the immunomodulatory molecules of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kan^{R}(U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon.

Typically, the host cell should secrete minimal amounts of proteolytic enzymes, and additional protease inhibitors may desirably be incorporated in the cell culture.

### c) Protein Production

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the immunomodulatory molecules of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

The expressed immunomodulatory molecules of the present application are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small-scale fermentation refers generally to fermentation in a fermentor that is no more than approximately 100 liters in volumetric capacity, and can range from about 1 liter to about 100 liters.

During the fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the immunomodulatory molecules of the present application, various fermentation conditions can be modified. For example, to improve the proper assembly and folding of the secreted polypeptides, additional vectors overexpressing chaperone proteins, such as Dsb proteins (DsbA, DsbB, DsbC, DsbD, or DsbG) or FkpA (a peptidylprolyl cis, trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

E. coli strains deficient for proteolytic enzymes and transformed with plasmids overexpressing one or more chaperone proteins may be used as host cells in the expression system encoding the immunomodulatory molecules of the present application.

### d) Protein Purification

The immunomodulatory molecules produced herein are further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In some embodiments, Protein A immobilized on a solid phase is used for immunoaffinity purification of the immunomodulatory molecules comprising an Fc region of the present application. Protein A is a 42 kDa surface protein from Staphylococcus aureus which binds with a high affinity to Fc-containing constructs, e.g., antigen-binding fragment-hinge-Fc fusion proteins, antibodies, or immunomodulatory molecules described herein. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants. The solid phase is then washed to remove contaminants non-specifically bound to the solid phase. Finally, the immunomodulatory molecules of interest are recovered from the solid phase by elution.

### 2. Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

### a) Signal Sequence Component

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region is ligated in reading frame to DNA encoding the immunomodulatory molecules of the present application.

### b) Origin of Replication

Generally, the origin of replication component is not needed for mammalian expression vectors (the SV40 origin may typically be used only because it contains the early promoter).

### c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the immunomodulatory molecules of the present application, such as DHFR, thymidine kinase, metallothionein-I and —II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

### d) Promoter Component

Expression and cloning vectors usually contain a promoter that is recognized by the host organism and is operably linked to the nucleic acid encoding the desired polypeptide sequences. Virtually all eukaryotic genes have an AT-rich region located approximately 25 to 30 based upstream from the site where transcription is initiated. Another sequence found 70 to 80 bases upstream from the start of the transcription of many genes is a CNCAAT region where N may be any nucleotide. At the 3′ end of most eukaryotic is an AATAAA sequence that may be the signal for addition of the poly A tail to the 3′ end of the coding sequence. All of these sequences may be inserted into eukaryotic expression vectors. Also see “Promoters” subsection under “III. Vectors encoding immunomodulatory molecules” above.

Polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human-interferon cDNA in mouse cells under the control of a thymidine kinase promoter from herpes simplex virus. Alternatively, the Rous Sarcoma Virus long terminal repeat can be used as the promoter.

### e) Enhancer Element Component

Transcription of a DNA encoding the immunomodulatory molecules of the present application by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (100-270 bp), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

### f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region. See WO94/11026 and the expression vector disclosed therein.

### g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for immunomodulatory molecule production and cultured in conventional nutrient media modified as appropriate for inducing promoters, selecting transformants, or amplifying the genes encoding the desired sequences.

### h) Culturing the Host Cells

The host cells used to produce the immunomodulatory molecules of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### i) Protein Purification

When using recombinant techniques, the immunomodulatory molecule can be produced intracellularly, in the periplasmic space, or directly secreted into the medium. If the immunomodulatory molecule is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the immunomodulatory molecule is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the immunomodulatory molecule. Protein A can be used to purify the immunomodulatory molecules, antigen-binding fragment-Fc fusion proteins, or antibodies that are based on human immunoglobulins containing 1, 2, or 4 heavy chains (Lindmark et al., J. Immunol. Meth. 62:1-13 (1983)). Protein G is recommended for all mouse isotypes and for human 3 (Guss et al., EMBO J. 5:15671575 (1986)). The matrix to which the affinity ligand is attached is most often agarose, but other matrices are available. Mechanically stable matrices such as controlled pore glass or poly(styrene-divinyl) benzene allow for faster flow rates and shorter processing times than can be achieved with agarose. Where the immunomodulatory molecule comprises a C_{H}3 domain, the Bakerbond ABXTMresin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the immunomodulatory molecule to be recovered.

Following any preliminary purification step(s), the mixture comprising the immunomodulatory molecule of interest and contaminants may be subjected to low pH hydrophobic interaction chromatography using an elution buffer at a pH between about 2.54.5, preferably performed at low salt concentrations (e.g., from about 0-0.25M salt).

### V. Pharmaceutical Compositions

Further provided are pharmaceutical compositions comprising any of the immunomodulatory molecules described herein (such as described in any of FIGS. 1A-1W and 11A-15D, Examples, and Sequence Listing herein, e.g., IL-2/anti-PD-1 agonist Ab immunomodulatory molecule, IL-12/anti-PD-1 agonist Ab immunomodulatory molecule, IL-2/PD-L1 immunomodulatory molecule, IL-12/PD-L1 immunomodulatory molecule, IL-2/PD-L2 immunomodulatory molecule, IL-12/PD-L2 immunomodulatory molecule), and optionally a pharmaceutically acceptable carrier. Pharmaceutical compositions can be prepared by mixing an immunomodulatory molecule described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

A reconstituted formulation can be prepared by dissolving a lyophilized immunomodulatory molecule described herein in a diluent such that the protein is dispersed throughout. Exemplary pharmaceutically acceptable (safe and non-toxic for administration to a human) diluents suitable for use in the present application include, but are not limited to, sterile water, bacteriostatic water for injection (BWFI), a pH buffered solution (e.g., phosphate-buffered saline), sterile saline solution, Ringer's solution or dextrose solution, or aqueous solutions of salts and/or buffers.

In some embodiments, the pharmaceutical composition comprises a homogeneous population of immunomodulatory molecules described herein. A homogeneous population means the immunomodulatory molecules are exactly the same to each other, e.g., same immunomodulatory molecule configuration, same first binding domain (e.g., cytokine moiety), same second binding domain (e.g., ligand, receptor, VHH, scFv, or Fab), same linker if any, same hinge region, and same Fc domain. In some embodiments, at least about 70% (such as at least about any of 75%, 80%, 85%, 90%, 95%, 96%, 97%, 98%, or 99%) of the immunomodulatory molecules in the pharmaceutical composition are homogeneous.

The pharmaceutical composition is preferably to be stable, in which the immunomodulatory molecule here essentially retains its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For rapid screening, the formulation may be kept at 40° C. for 2 weeks to 1 month, at which time stability is measured. Where the formulation is to be stored at 2-8° C., generally the formulation should be stable at 30° C. or 40° C. for at least 1 month, and/or stable at 2-8° C. for at least 2 years. Where the formulation is to be stored at 30° C., generally the formulation should be stable for at least 2 years at 30° C., and/or stable at 40° C. for at least 6 months. For example, the extent of aggregation during storage can be used as an indicator of protein stability. In some embodiments, the stable formulation of immunomodulatory molecules described herein may comprise less than about 10% (preferably less than about 5%) of the immunomodulatory molecules present as an aggregate in the formulation.

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g., sodium chloride), stabilizers, metal complexes (e.g., Zn-protein complexes); chelating agents such as EDTA and/or non-ionic surfactants.

Examples of physiologically acceptable carriers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or nonionic surfactants such as TWEEN™, polyethylene glycol (PEG), and PLURONICS™ or polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use in the present application include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.00% (w/v). The addition of a preservative may, for example, facilitate the production of a multi-use (multiple dose) formulation. Suitable preservatives for use in the present application include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between 0.1% to 25% by weight, preferably 1% to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) are present to help solubilize the immunomodulatory molecules as well as to protect the immunomodulatory molecules against agitation-induced aggregation, which also permits the formulation to be exposed to shear surface stress without causing denaturation of the active immunomodulatory molecules. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they must be sterile. The pharmaceutical composition may be rendered sterile by filtration through sterile filtration membranes. The pharmaceutical compositions herein generally are placed into a container having a sterile access port, for example, an intravenous solution bag or vial having a stopper pierceable by a hypodermic injection needle.

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The pharmaceutical compositions herein may also contain more than one active compound as necessary for the particular indication being treated, preferably those with complementary activities that do not adversely affect each other. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 18th edition.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

### VI. Methods of Treating Diseases or Directing Cytokine Activity

The immunomodulatory molecules described herein (such as described in any of FIGS. 1A-1W and 11A-15D, Examples, and Sequence Listing herein, e.g., IL-2/anti-PD-1 agonist Ab immunomodulatory molecule, IL-12/anti-PD-1 agonist Ab immunomodulatory molecule, IL-2/PD-L1 immunomodulatory molecule, IL-12/PD-L1 immunomodulatory molecule, IL-2/PD-L2 immunomodulatory molecule, IL-12/PD-L2 immunomodulatory molecule) and compositions (e.g., pharmaceutical compositions) thereof are useful for a variety of applications, such as in diagnosis, molecular assays, and therapy. In some embodiments, there is provided a method of treating a disease (e.g., cancer (e.g., PD-L1+ and/or PD-L2+cancer), infection such as viral infection, autoimmune disease, allergy, graft rejection, or GvHD) in an individual (e.g., human), comprising administering to the individual an effective amount of any of the immunomodulatory molecules described herein or pharmaceutical compositions thereof. In some embodiments, there is also provided a method of modulating an immune response in an individual (e.g., human), comprising administering to the individual an effective amount of any of the immunomodulatory molecules described herein or pharmaceutical compositions thereof. In some embodiments, the activity of the first binding domain (e.g., cytokine or variant thereof) is selectively activated upon binding of the immunomodulatory molecule to the second target molecule, when the first binding domain is positioned at the hinge region between the second binding domain and an Fc domain or portion thereof. In some embodiments, the immunomodulatory molecule or pharmaceutical composition thereof is administered intravenously, subcutaneously, or intratumorally. In some embodiments, the immunomodulatory molecule or pharmaceutical composition thereof is administered in an amount of about 1 μg/kg to about 10 mg/kg. In some embodiments, the immunomodulatory molecule or pharmaceutical composition thereof is administered once every three weeks. In some embodiments, the cancer is selected from the group consisting of lung cancer, liver cancer, renal cancer, colorectal cancer, ovarian cancer, breast cancer, pancreatic cancer, gastric carcinoma, bile duct cancer, squamous cell carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphoma, myeloma, mycoses fungoides, and merkel cell cancer.

In some embodiments, the method of treating cancer has one or more of the following biological activities: (1) killing cancer cells; (2) inhibiting proliferation of cancer cells; (3) inducing immune response in a tumor (e.g., inducing infiltration of immune effector cells to tumor site, inducing immune cell proliferation, differentiation and/or activation, and/or inducing pro-inflammatory cytokine secretion by immune cells); (4) reducing tumor size; (5) alleviating one or more symptoms in an individual having cancer; (6) inhibiting tumor metastasis; (7) prolonging survival; (8) prolonging time to cancer progression; and (9) preventing, inhibiting, or reducing the likelihood of the recurrence of a cancer. In some embodiments, the method of killing cancer cells mediated by the immunomodulatory molecule or pharmaceutical composition described herein can achieve a tumor cell death rate of at least about any of 10%, 20%, 30%, 40%, 50%, 60%, 70%, 80%, 90%, 95%, or more. In some embodiments, the method of reducing tumor size mediated by the immunomodulatory molecule or pharmaceutical composition described herein can reduce at least about 10% (including for example at least about any of 20%, 30%, 40%, 60%, 70%, 80%, 90%, or 100%) of the tumor size. In some embodiments, the method of inhibiting tumor metastasis mediated by the immunomodulatory molecule or pharmaceutical composition described herein can inhibit at least about 10% (including for example at least about any of 20%, 30%, 40%, 60%, 700%6, 80%, 90%, or 100%) of the metastasis. In some embodiments, the method of prolonging survival of an individual (e.g., human) mediated by the immunomodulatory molecule or pharmaceutical composition described herein can prolongs the survival of the individual by at least any of 1, 2, 3, 4, 5, 6, 7, 8, 9, 10, 11, 12, 18, or 24 months. In some embodiments, the method of prolonging time to cancer progression mediated by the immunomodulatory molecule or pharmaceutical composition described herein can prolong the time to cancer progression by at least any of 1, 2, 3, 4, 5, 6, 7, 8, 9, 10, 11, or 12 weeks. In some embodiments, the method of inducing immune response to a tumor can increase, enhance, or stimulate an immune response or function in a subject. In some embodiments, the immune response or function is increased, enhanced, and/or stimulated by activating effector cells (e.g., T cells, e.g., CD8+ and/or CD4+ T cells), expanding (increasing) an effector cell population, and/or killing target cells (e.g., target tumor cells) in the subject. In some embodiments, the CD4 and/or CD8 T cells in the individual have increased or enhanced priming, activation, proliferation, cytokine release and/or cytolytic activity relative to prior to the administration of the immunomodulatory molecule or pharmaceutical composition described herein.

The methods described herein are suitable for treating a variety of cancers, including both solid cancer and liquid cancer. The methods are applicable to cancers of all stages, including early stage cancer, non-metastatic cancer, primary cancer, advanced cancer, locally advanced cancer, metastatic cancer, or cancer in remission. The methods described herein may be used as a first therapy, second therapy, third therapy, or combination therapy with other types of cancer therapies known in the art, such as surgery, radiation, chemotherapy, immunotherapy, hormone therapy, or a combination thereof. In some embodiments, the method is used to treat an individual who has previously been treated. In some embodiments, the cancer has been refractory to prior therapy. In some embodiments, the method is used to treat an individual who has not previously been treated. In some embodiments, the cancer is partially resistant to immune checkpoint inhibitor monotherapy (e.g., partially resistant to anti-PD-1 or anti-PD-L1 antibody monotherapy treatment).

In some embodiments, the cancer is a PD-L1 expressing cancer. In some embodiments, the method is suitable for treating cancers with aberrant PD-1 or PD-L1/PD-L2 expression (e.g., HER2+cancer), activity and/or signaling include, by way of non-limiting example, hematological cancer and/or solid tumors. Some cancers whose growth may be inhibited using the immunomodulatory molecules of the invention include cancers typically responsive to immunotherapy. Non-limiting examples of other cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, the invention includes refractory or recurrent malignancies whose growth may be inhibited using the immunomodulatory molecules of the invention. The present invention is also useful for treatment of metastatic cancers, especially metastatic cancers that express PD-L1 (Iwai et al. (2005) Int. Immunol. 17:133-144). In some embodiments, the cancer with aberrant PD-1 or PD-L1/PD-L2 expression, activity and/or signaling is partially resistant to PD-1 or PD-L1 blockade (e.g., partially resistant to anti-PD-1 antibody or anti-PD-L1 antibody treatment).

In some embodiments, the methods described herein are suitable for treating a solid cancer selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), cutaneous T-cell lymphoma (CTCL), cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In some embodiments, the methods described herein are suitable for treating a hematologic cancer chosen from one or more of acute myeloid leukemia (AML), chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the methods described herein are for treating infection, e.g., fungal, viral, bacterial, protozoal, or other parasitic infection. In some embodiments, the method of treating infection described herein prevent worsening of, arrest and/or ameliorate at least one symptom of a pathogen infection in an individual in need thereof, reduce or eliminate pathogen, prevent damage to said individual or an organ or tissue of said individual, and/or prevent death. In some embodiments, the methods described herein can achieve one or more of the following: (a) controlling, ameliorating, and/or preventing tissue and/or organ injury or failure, such as induced by virus infection; (b) controlling, reducing, and/or inhibiting cell necrosis (such as reducing at least about 10% (including for example at least about any of 20%, 30%, 40%, 60%, 70%, 80%, 90%, or 100%) cell necrosis), such as necrosis in infected and/or non-infected tissue and/or organ; (c) controlling, and/or increasing the infiltration of inflammatory cells (e.g., NK cells, cytotoxic T cells, neutrophils) in infected tissues and/or organs, such as increasing at least about 10% (including for example at least about any of 20%, 30%, 40%, 60%, 70%, 80%, 90%, or 100%) inflammatory cell infiltration; (d) controlling, ameliorating and/or preventing inflammation in non-infected tissue and/or organ, systemic inflammation, and/or cytokine storm, such as downregulating at least about 10% (including for example at least about any of 20%, 30%, 40%, 60%, 70%, 80%, 90%, or 100%); (e) reducing mortality rate associated with pathogen infection, and/or preventing death, such as reducing at least about 10% (including for example at least about any of 20%, 30%, 40%, 60%, 70%, 80%, 90%, or 100%) death rate; and (f) reducing or eliminating at least about 10% (including for example at least about any of 20%, 30%, 40%, 60%, 70%, 80%, 90%, or 100%) pathogen.

In some embodiments, the methods described herein are for treating an immune disease, such as an autoimmune disease, or an immune suppression.

In some embodiments, the methods described herein are for treating immune suppression. Immunosuppression is a reduction or entirely absent of the activation or efficacy of the immune system, resulting in immune system's inability to fight diseases, for example infectious diseases or cancer. Immunosuppression can either be the result of diseases, or be produced by pharmaceuticals or an infection, resulting in an increased susceptibility to secondary infections by pathogens such as bacteria and viruses. Many diseases are characterized by the development of progressive immunosuppression in the patient. The presence of an impaired immune response in patients with malignancies (e.g. leukemia, lymphoma, multiple myeloma) is well documented. Progressive immunosuppression has also been observed in certain chronic infection such as AIDS, sepsis, leprosy, cytomegalovirus infections, malaria, lupus, and the like. Immunodeficiency is also a potential adverse effect of many therapeutic treatments (radiotherapy or chemotherapy for example). By means of example and not limitation, diseases and conditions associated with immunodeficiency or immunosuppression comprise: human immunodeficiency virus (HIV) infection and acquired immune deficiency syndrome (AIDS), hypogammaglobulinemia, hematologic cancers such as leukaemia and lymphoma, lymphocytopenia (lymphopenia) of any origin, lupus erythematosus, cachexia, opioids abuse, mastocytosis, rheumatic fever, trypanosomiasis, and alcohol abuse. In some embodiments, immunosuppression is associated with immune checkpoint signaling (e.g., PD-1 or CTLA-4 signaling). In such non-deliberate immunosuppression situations, patients are usually treated with immunostimulants (e.g. cytokines) to boost immune system. However, due to the lack of specificity, such immunostimulants activate the immune system in general and may trigger an overactivation of the immune system.

In some embodiments, the methods of treating an immune suppression described herein activate or enhance immune response, increase CD8 to CD4 ratio, promote immune cell proliferation and/or differentiation, induce or enhance cytokine release (e.g., IL-2, IL-6, IFN-γ), prevent worsening of, arrest and/or ameliorate at least one symptom of an immune suppression in an individual in need thereof, and/or prevent death.

In some embodiments, the methods described herein are for treating autoimmune diseases. Autoimmune disease is a disease resulting from an immune response against a self-tissue or tissue component, including both self-antibody responses and cell-mediated responses. The term “autoimmune disease,” as used herein, encompasses organ-specific autoimmune diseases, in which an autoimmune response is directed against a single tissue, such as type I diabetes mellitus (T1D), Crohn's disease, ulcerative colitis, myasthenia gravis, vitiligo, Graves' disease, Hashimoto's disease, Addison's disease and autoimmune gastritis and autoimmune hepatitis. The term “autoimmune disease” also encompasses non-organ specific autoimmune diseases, in which an autoimmune response is directed against a component present in several or many organs throughout the body. Such autoimmune diseases include, for example, rheumatoid disease, systemic lupus erythematosus, progressive systemic sclerosis and variants, polymyositis and dermatomyositis. Additional autoimmune diseases include pernicious anemia including some of autoimmune gastritis, primary biliary cirrhosis, autoimmune thrombocytopenia, Sjogren's syndrome, multiple sclerosis and psoriasis. In some embodiments, the autoimmune disease is selected from the group consisting of diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, inflammatory bowel disease (IBD), Crohn's disease, Graves ophthalmopathy, sarcoidosis, primary biliary cirrhosis, uveitis posterior, and interstitial lung fibrosis. One skilled in the art understands that the methods of the invention can be applied to these or other autoimmune diseases, as desired.

In some embodiments, the methods of treating an autoimmune disease described herein prevent worsening of, arrest and/or ameliorate at least one symptom of an autoimmune disease in an individual in need thereof, prevent damage to healthy self tissues or organ, control, ameliorate and/or prevent infiltration of immune cells to healthy self tissue and/or organ, systemic inflammation, and/or cytokine storm, and/or prevent death.

In some embodiments, the methods of treating a graft rejection described herein prevent worsening of, arrest and/or ameliorate at least one symptom of a graft rejection in an individual in need thereof; prevent damage to donor/foreign tissues or organ; control, ameliorate and/or prevent infiltration of immune cells to donor/foreign tissues or organ, systemic inflammation, and/or cytokine storm; reduce Th17 cell activation; improve graft survival; prolong survival, increase survival rate, and/or prevent death. In some embodiments, the methods of treating a GvHD described herein prevent worsening of, arrest and/or ameliorate at least one symptom of a GvHD in an individual in need thereof; reduce Th17 cell activation; prevent damage to self/healthy tissues or organ; control, ameliorate and/or prevent infiltration of immune cells to self/healthy tissues or organ, systemic inflammation, and/or cytokine storm; improve graft survival; prolong survival, increase survival rate, and/or prevent death; and/or improve disease activity score (see, e.g., P. J. Martin, Biol Blood Marrow Transplant. 2009 July; 15(7):777-784).

In some embodiments, there is provided a method of selectively activating the activity (binding affinity to corresponding cytokine receptor or subunit thereof, and/or biological activity) of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD123, CD25, HER2, PD-1, CD3, CD4, or CD8) in an individual (e.g., human), comprising administering to the individual an effective amount of an immunomodulatory molecule (or pharmaceutical compositions thereof), wherein the immunomodulatory molecule comprises: a) an antigen-binding protein (e.g., antibody such as full-length antibody, or antigen-binding fragment-hinge-Fc fusion protein such as ligand/receptor-hinge-Fc fusion protein) specifically recognizing a target antigen (e.g., CTLA-4, PD-L1, PD-L2, CD123, CD25, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antigen-binding protein comprises an antigen-binding polypeptide (e.g., antibody heavy chain, or antigen-binding fragment-hinge-Fc fusion polypeptide such as ligand/receptor-hinge-Fc fusion polypeptide) comprising from N′ to C′: an antigen-binding fragment (e.g., ligand, receptor, VHH, scFv, or VH), a hinge region, and an Fc domain subunit or portion thereof (e.g., CH2+CH3, or CH2 only), wherein the cytokine or variant thereof is positioned at (e.g., at the N′ of, at the C′ of, or within) the hinge region; and wherein the activity of the cytokine or variant thereof is selectively activated upon binding of the antigen-binding protein to the target antigen. In some embodiments, there is provided a method of selectively activating the activity (binding affinity to corresponding cytokine receptor or subunit thereof, and/or biological activity) of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD25, CD123, HER2, PD-1, CD3, CD4, or CD8) in an individual (e.g., human), comprising administering to the individual an effective amount of an immunomodulatory molecule (or pharmaceutical compositions thereof), wherein the immunomodulatory molecule comprises: a) an antibody (e.g., full-length antibody, heavy chain only antibody, or antigen-binding fragment fused to an Fc domain subunit or portion thereof via a hinge region) specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising a hinge region, and wherein the cytokine or variant thereof is positioned at the hinge region (e.g., within the hinge region, or between the C-terminus of CH1 and the N-terminus of the hinge region) of the heavy chain; and wherein the activity of the cytokine or variant thereof is selectively activated upon binding of the antibody to the target antigen. In some embodiments, there is provided a method of selectively activating the activity (binding affinity to corresponding cytokine receptor or subunit thereof, and/or biological activity) of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD25, CD123, HER2, PD-1, CD3, CD4, or CD8) in an individual (e.g., human), comprising administering to the individual an effective amount of an immunomodulatory molecule (or pharmaceutical compositions thereof), wherein the immunomodulatory molecule comprises: a) an antibody (e.g., full-length antibody, or antigen-binding fragment fused to an Fc domain subunit or portion thereof via a hinge region) specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the antibody comprises a heavy chain comprising from N-terminus to C-terminus: a VH domain, optionally a CH1 domain, the cytokine or variant thereof at a hinge region, a CH2 domain, and optionally a CH3 domain; and wherein the activity of the cytokine or variant thereof is selectively activated upon binding of the antibody to the target antigen. In some embodiments, there is provided a method of selectively activating the activity (binding affinity to corresponding cytokine receptor or subunit thereof, and/or biological activity) of a cytokine or variant thereof (e.g., IL-2, IFN-α (e.g., IFN-α2b), IFN-γ, IL-10, IL-12, or IL-23) to a cell expressing a target antigen (e.g., CTLA-4, PD-L1, PD-L2, CD25, CD123, HER2, PD-1, CD3, CD4, or CD8) in an individual (e.g., human), comprising administering to the individual an effective amount of an immunomodulatory molecule (or pharmaceutical compositions thereof), wherein the immunomodulatory molecule comprises: a) a full-length antibody specifically recognizing a target antigen (e.g., CTLA-4, PD-L1, PD-L2, CD25, CD123, HER2, PD-1, CD3, CD4, or CD8); and b) a cytokine (e.g., IL-2, IFN-α (e.g., IFN-α2b), IFN-γ, IL-10, IL-12, or IL-23) or variant thereof, wherein the cytokine or variant thereof is positioned at the hinge region (e.g., within the hinge region, or between the C-terminus of CH1 and the N-terminus of the hinge region) of a heavy chain of the full-length antibody; and wherein the activity of the cytokine or variant thereof is selectively activated upon binding of the full-length antibody to the target antigen. In some embodiments, in the presence of binding of the antigen-binding protein (e.g., antibody such as full-length antibody, or antigen-binding fragment-hinge-Fc fusion protein such as ligand/receptor-hinge-Fc fusion protein) or antigen-binding fragment (e.g., ligand, receptor, V_{H}H, scFv, Fab) to the target antigen, the activity (binding affinity to corresponding cytokine receptor or subunit thereof, and/or biological activity) of the cytokine or variant thereof increases at least about 20% (such as at least about any of 30%, 40%, 50%, 60%, 70%, 80%, 90%, 100%, 200%, 300%, 400%, 500%, or more) compared to that in the absence of binding of the antigen-binding protein (e.g., antibody such as full-length antibody, or antigen-binding fragment-hinge-Fc fusion protein such as ligand/receptor-hinge-Fc fusion protein) or antigen-binding fragment (e.g., ligand, receptor, VHH, scFv, Fab) to the target antigen. In some embodiments, in the absence of binding of the antigen-binding protein (e.g., antibody such as full-length antibody, or antigen-binding fragment-hinge-Fc fusion protein such as ligand/receptor-hinge-Fc fusion protein or antigen-binding fragment (e.g., ligand, receptor, VHH, scFv, Fab) to the target antigen, the activity (binding affinity to corresponding cytokine receptor or subunit thereof, and/or biological activity) of the cytokine or variant thereof positioned at the hinge region of the heavy chain is no more than about 70% (such as no more than about any of 60%, 50%, 40%, 30%, 20%, 10%, 9%, 8%, 7%, 6%, 5%, 4%, 3%, 2%, 1%, 0.9%,0.8%, 0.7%, 0.6%, 0.5%, 0.4%, 0.3%, 0.2%, 0.1%, or 0%) of that of a corresponding cytokine or variant thereof in a free state. In some embodiments, the cytokine or variant thereof is a cytokine variant, and wherein the activity (binding affinity to corresponding cytokine receptor or subunit thereof, and/or biological activity) of the cytokine variant in a free state is no more than about 80% (such as no more than about any of 70%, 60%, 500/, 40%, 30%, 20%, 10%, or 5%) of that of a corresponding wildtype cytokine in a free state.

Administration of the immunomodulatory molecules described herein or pharmaceutical compositions thereof may be carried out in any convenient manner, including by injection or transfusion. The route of administration is in accordance with known and accepted methods, such as by single or multiple bolus or infusion over a long period of time in a suitable manner. The immunomodulatory molecules or pharmaceutical compositions thereof may be administered to a patient transarterially, subcutaneously, intradermally, intratumorally, intranodally, intramedullary, intramuscularly, intravenously, or intraperitoneally. In some embodiments, the immunomodulatory molecule or pharmaceutical composition thereof is administered systemically. In some embodiments, the immunomodulatory molecule or pharmaceutical composition thereof is administered to an individual by infusion, such as intravenous infusion. Infusion techniques for immunotherapy are known in the art (see, e.g., Rosenberg el al., New Eng. J. of Med. 319: 1676 (1988)). In some embodiments, the immunomodulatory molecule or pharmaceutical composition thereof is administered to an individual by intradermal or subcutaneous (i.e., beneath the skin) injection. For subcutaneous injections, the immunomodulatory molecules or pharmaceutical compositions may be injected using a syringe. However, other devices for administration of the immunomodulatory molecules or pharmaceutical compositions are available such as injection devices; injector pens; auto-injector devices, needleless devices; and subcutaneous patch delivery systems. In some embodiments, the immunomodulatory molecule or pharmaceutical composition thereof is administered by intravenous injection. In some embodiments, the immunomodulatory molecule or pharmaceutical composition thereof is injected directly into a tumor, or a lymph node. In some embodiments, the immunomodulatory molecule or pharmaceutical composition thereof is administered locally to a site of tumor, such as directly into tumor cells, or to a tissue having tumor cells. In some embodiments, the immunomodulatory molecule or pharmaceutical composition thereof is administered by sustained release or extended-release means.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46. It is within the scope of the present application that different formulations will be effective for different treatments and different disorders, and that administration intended to treat a specific organ or tissue may necessitate delivery in a manner different from that to another organ or tissue.

When in vivo administration of the immunomodulatory molecules described herein or pharmaceutical compositions thereof are used, normal dosage amounts may vary from about 1 μg/kg to about 10 mg/kg of mammal body weight depending upon the route of administration and mammal type. It is within the scope of the present application that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays. In some embodiments, the immunomodulatory molecule described herein or pharmaceutical composition thereof is administered in an amount of about 1 μg/kg to about 10 mg/kg, such as any of about 1 μg/kg to about 500 μg/kg, about 500 μg/kg to about 1 mg/kg, about 1 mg/kg to about 10 mg/kg, about 1 μg/kg to about 1 mg/kg, about 1 μg/kg to about 200 μg/kg, about 100 μg/kg to about 500 μg/kg, about 100 μg/kg to about 1 mg/kg, or about 500 μg/kg to about 1 mg/kg.

In some embodiments, the immunomodulatory molecule described herein or pharmaceutical composition thereof is administered (e.g., infused) to the individual (e.g., human) over a period of time no more than about any of 24 hours, 20 hours, 15 hours, 10 hours, 8 hours, 6 hours, 3 hours, 2 hours, 1 hours, 30 minutes, or less. In some embodiments, the immunomodulatory molecule described herein or pharmaceutical composition thereof is administered (e.g., infused) to the individual (e.g., human) over a period of time of any one of about 30 minutes to about 1 hour, about 1 hour to about 2 hours, about 2 hours to about 4 hours, about 4 hours to about 6 hours, about 6 hours to about 8 hours, about 8 hours to about 10 hours, about 10 hours to about 12 hours, about 12 hours to about 18 hours, about 18 hours to about 24 hours, about 30 minutes to about 2 hours, about 2 hours to about 5 hours, about 5 hours to about 10 hours, about 10 hours to about 20 hours, about 30 minutes to about 10 hours, or about 30 minutes to about 24 hours.

In some embodiments, the immunomodulatory molecule described herein or pharmaceutical composition thereof is administered for a single time (e.g., bolus injection). In some embodiments, the immunomodulatory molecule described herein or pharmaceutical composition thereof is administered for multiple times (such as any of 2, 3, 4, 5, 6, or more times). If multiple administrations, they may be performed by the same or different routes and may take place at the same site or at alternative sites. The immunomodulatory molecule described herein or pharmaceutical composition thereof may be administered daily to once per year. The interval between administrations can be about any one of 24 hours to a year. Intervals can also be irregular (e.g., following tumor progression). In some embodiments, there is no break in the dosing schedule. The optimal dosage and treatment regime for a particular patient can readily be determined by one skilled in the art of medicine by monitoring the patient for signs of disease and adjusting the treatment accordingly. In some embodiments, the immunomodulatory molecule described herein or pharmaceutical composition thereof is administered once per day (daily), once per 2 days, once per 3 days, once per 4 days, once per 5 days, once per 6 days, once per week, once per 10 days, once every 2 weeks, once every 3 weeks, once every 4 weeks, once per month, once per 2 months. once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. In some embodiments, the immunomodulatory molecule described herein or pharmaceutical composition thereof is administered once every three weeks.

In some embodiments, the pharmaceutical composition is administered in split doses, such as about any one of 2, 3, 4, 5, or more doses. In some embodiments, the split doses are administered over about a week, a month, 2 months, 3 months, or longer. In some embodiments, the dose is equally split. In some embodiments, the split doses are about 20%, about 30% and about 50% of the total dose. In some embodiments, the interval between consecutive split doses is about 1 day, 2 days, 3 days, 1 week, 2 weeks, 3 weeks, a month, or longer. For repeated administrations over several days or longer, depending on the condition, the treatment is sustained until a desired suppression of disease symptoms occurs. However, other dosage regimens may be useful. The progress of this therapy is easily monitored by conventional techniques and assays.

### VII. Articles of Manufacture and Kits

Further provided are kits, unit dosages, and articles of manufacture comprising any of the immunomodulatory molecules described herein (such as described in any of FIGS. 1A-1W and 11A-15D, Examples, and Sequence Listing herein). In some embodiments, a kit is provided which contains any one of the pharmaceutical compositions described herein and preferably provides instructions for its use, such as for use in the treatment of the disorders described herein (e.g., cancer, infection, or autoimmune disease).

Kits of the invention include one or more containers comprising an immunomodulatory molecule described herein for treating a disease. For example, the instructions comprise a description of administration of the immunomodulatory molecule to treat a disease, such as cancer. The kit may further comprise a description of selecting an individual (e.g., human) suitable for treatment based on identifying whether that individual has the disease and the stage of the disease. The instructions relating to the use of the immunomodulatory molecule generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The kits of the present application are in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. A kit may have a sterile access port (for example, the container may be an intravenous solution bag or a vial having a stopper pierceable by a hypodermic injection needle). At least one active agent in the composition is an immunomodulatory molecule as described herein. The container may further comprise a second pharmaceutically active agent. The kits may optionally provide additional components such as buffers and interpretive information. Normally, the kit comprises a container and a label or package insert(s) on or associated with the container.

The present application thus also provides articles of manufacture, which include vials (such as sealed vials), bottles, jars, flexible packaging, and the like. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example, the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

### EXEMPLARY EMBODIMENTS

Embodiment 1. An immunomodulatory molecule comprising a first binding domain specifically recognizing a first target molecule and a second binding domain specifically recognizing a second target molecule, wherein the first binding domain upon binding to the first target molecule up-regulates an immune response, and wherein the second binding domain upon binding to the second target molecule down-regulates the immune response.

Embodiment 2. The immunomodulatory molecule of embodiment 1, wherein the first binding domain upon binding to the first target molecule up-regulates the immune response by an activity (“up-regulated activity”) selected from one or more of up-regulating release of an immunostimulatory cytokine, down-regulating release of an immunosuppressive cytokine, up-regulating immune cell proliferation, up-regulating immune cell differentiation, up-regulating immune cell activation, up-regulating cytotoxicity against a tumor cell, and up-regulating elimination of an infectious agent.

Embodiment 3. The immunomodulatory molecule of embodiment 1 or 2, wherein the second binding domain upon binding to the second target molecule down-regulates the immune response by an activity (“down-regulated activity”) selected from one or more of down-regulating release of an immunostimulatory cytokine, up-regulating release of an immunosuppressive cytokine, down-regulating immune cell proliferation, down-regulating immune cell differentiation, down-regulating immune cell activation, down-regulating cytotoxicity against a tumor cell, and down-regulating elimination of an infectious agent.

Embodiment 4. The immunomodulatory molecule of any one of embodiments 1-3, wherein the immunostimulatory cytokine is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF.

Embodiment 5. The immunomodulatory molecule of any one of embodiments 1-4, wherein the immunosuppressive cytokine is selected from the group consisting of IL-1Ra, IL-4, IL-5, IL-6, IL-10, IL-11, IL-13, IL-27, IL-33, IL-35, IL-37, IL-39, IFN-α, LIF, and TGF-β.

Embodiment 6. The immunomodulatory molecule of any one of embodiments 1-5, wherein the first target molecule and/or the second target molecule is a stimulatory checkpoint molecule.

Embodiment 7. The immunomodulatory molecule of embodiment 6, wherein the stimulatory checkpoint molecule is selected from the group consisting of CD27, CD28, CD40, CD122, CD137, OX40, GITR, and ICOS.

Embodiment 8. The immunomodulatory molecule of embodiment 6 or 7, wherein the first binding domain is an agonist antibody or antigen-binding fragment thereof.

Embodiment 9. The immunomodulatory molecule of embodiment 6 or 7, wherein the first binding domain is an agonist ligand or variant thereof.

Embodiment 10. The immunomodulatory molecule of embodiment 9, wherein the agonist ligand is selected from the group consisting of CD27L (TNFSF7, CD70), CD40L (CD154), CD80, CD86, CD137L, OX40L (CD252), GITRL, and ICOSLG (CD275).

Embodiment 11. The immunomodulatory molecule of embodiment 9 or 10, wherein the first binding domain is a variant of an agonist ligand, and wherein the variant of the agonist ligand has increased or decreased binding affinity to the first target molecule compared to the agonist ligand.

Embodiment 12. The immunomodulatory molecule of any one of embodiments 6-11, wherein the second binding domain is an antagonist antibody or antigen-binding fragment thereof.

Embodiment 13. The immunomodulatory molecule of any one of embodiments 6-11, wherein the second binding domain is an antagonist ligand or variant thereof.

Embodiment 14. The immunomodulatory molecule of embodiment 13, wherein the second binding domain is a variant of an antagonist ligand, and wherein the variant of the antagonist ligand has increased or decreased binding affinity to the second target molecule compared to the antagonist ligand.

Embodiment 15. The immunomodulatory molecule of any one of embodiments 1-5, wherein the first target molecule and/or the second target molecule is a receptor of an immunostimulatory cytokine.

Embodiment 16. The immunomodulatory molecule of embodiment 15, wherein the immunostimulatory cytokine is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF.

Embodiment 17. The immunomodulatory molecule of embodiment 15 or 16, wherein the first binding domain is the immunostimulatory cytokine or variant thereof.

Embodiment 18. The immunomodulatory molecule of embodiment 17, wherein the first binding domain is a variant of an immunostimulatory cytokine, and wherein the variant of the immunostimulatory cytokine has increased or decreased binding affinity to the first target molecule compared to the immunostimulatory cytokine.

Embodiment 19. The immunomodulatory molecule of embodiment 17 or 18, wherein the first binding domain is IL-12, IL-2, or variant thereof.

Embodiment 20. The immunomodulatory molecule of embodiment 15 or 16, wherein the first binding domain is an agonist antibody or antigen-binding fragment thereof.

Embodiment 21. The immunomodulatory molecule of any one of embodiments 15-20, wherein the second binding domain is an antagonist antibody or antigen-binding fragment thereof.

Embodiment 22. The immunomodulatory molecule of any one of embodiments 15-20, wherein the second binding domain is antagonist ligand or variant thereof.

Embodiment 23. The immunomodulatory molecule of embodiment 22, wherein the second binding domain is a variant of an antagonist ligand, and wherein the variant of the antagonist ligand has increased or decreased binding affinity to the second target molecule compared to the antagonist ligand.

Embodiment 24. The immunomodulatory molecule of any one of embodiments 1-5, wherein the first target molecule and/or the second target molecule is an activating immune cell surface receptor.

Embodiment 25. The immunomodulatory molecule of embodiment 24, wherein the activating immune cell surface receptor is selected from the group consisting of CD2, CD3, CD4, CD8, CD16, CD56, CD96, CD161, CD226, NKG2C, NKG2D, NKG2E, NKG2F, NKG2H, NKp30, NKp44, NKp46, CD11c, CD11b, CD13, CD45RO, CD33, CD123, CD62L, CD45RA, CD3δ, CD163, and CD206.

Embodiment 26. The immunomodulatory molecule of embodiment 24 or 25, wherein the first binding domain is an agonist antibody or antigen-binding fragment thereof.

Embodiment 27. The immunomodulatory molecule of embodiment 24 or 25, wherein the first binding domain is an agonist ligand or variant thereof.

Embodiment 28. The immunomodulatory molecule of embodiment 27, wherein the first binding domain is a variant of an agonist ligand, and wherein the variant of the agonist ligand has increased or decreased binding affinity to the first target molecule compared to the agonist ligand.

Embodiment 29. The immunomodulatory molecule of any one of embodiments 24-28, wherein the second binding domain is an antagonist antibody or antigen-binding fragment thereof.

Embodiment 30. The immunomodulatory molecule of any one of embodiments 24-28, wherein the second binding domain is an antagonist ligand or variant thereof.

Embodiment 31. The immunomodulatory molecule of embodiment 30, wherein the second binding domain is a variant of an antagonist ligand, and wherein the variant of the antagonist ligand has increased or decreased binding affinity to the second target molecule compared to the antagonist ligand.

Embodiment 32. The immunomodulatory molecule of any one of embodiments 1-5, wherein the first target molecule and/or the second target molecule is an inhibitory checkpoint molecule.

Embodiment 33. The immunomodulatory molecule of embodiment 32, wherein the inhibitory checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, Siglec7, Siglec9, and VISTA.

Embodiment 34. The immunomodulatory molecule of embodiment 32 or 33, wherein the first binding domain is an antagonist ligand or variant thereof.

Embodiment 35. The immunomodulatory molecule of embodiment 34, wherein the first binding domain is a variant of an antagonist ligand, and wherein the variant of the antagonist ligand has increased or decreased binding affinity to the first target molecule compared to the antagonist ligand.

Embodiment 36. The immunomodulatory molecule of embodiment 32 or 33, wherein the first binding domain is an antagonist antibody or antigen-binding fragment thereof.

Embodiment 37. The immunomodulatory molecule of any one of embodiments 32-36, wherein the second binding domain is an agonist antibody or antigen-binding fragment thereof.

Embodiment 38. The immunomodulatory molecule of embodiment 37, wherein the agonist antibody or antigen-binding fragment thereof specifically recognizes PD-1, TIGIT, LAG-3, TIM-3, or CTLA-4.

Embodiment 39. The immunomodulatory molecule of any one of embodiments 32-36, wherein the second binding domain is an agonist ligand or variant thereof.

Embodiment 40. The immunomodulatory molecule of embodiment 39, (i) wherein the second target molecule is PD-1, and wherein the second binding domain is PD-L1, PD-L2, or variant thereof; (ii) wherein the second target molecule is TIGIT, and wherein the second binding domain is CD112, CD155, or variant thereof; (iii) wherein the second target molecule is LAG-3, and wherein the second binding domain is MHC II, LSECtin, or variant thereof; (iv) wherein the second target molecule is TIM-3, and wherein the second binding domain is Galectin-9, Caecam-1, HMGB-1, phosphatidylserine, or variant thereof; or (v) wherein the second target molecule is CTLA-4, and wherein the second binding domain is CD80, CD86, or variant thereof.

Embodiment 41. The immunomodulatory molecule of embodiment 40, wherein the second binding domain is a variant of an agonist ligand, and wherein the variant of the agonist ligand has increased or decreased binding affinity to the second target molecule compared to the agonist ligand.

Embodiment 42. The immunomodulatory molecule of any one of embodiments 39-41, wherein the second binding domain comprises an extracellular domain of the agonist ligand or variant thereof.

Embodiment 43. The immunomodulatory molecule of any one of embodiments 1-5, wherein the first target molecule and/or the second target molecule is a receptor of an immunosuppressive cytokine.

Embodiment 44. The immunomodulatory molecule of embodiment 43, wherein the immunosuppressive cytokine is selected from the group consisting of IL-1Ra, IL-4, IL-5, IL-6, IL-10, IL-11, IL-13, IL-27, IL-33, IL-35, IFN-α, LIF, and TGF-β.

Embodiment 45. The immunomodulatory molecule of embodiment 43 or 44, wherein the second binding domain is the immunosuppressive cytokine or variant thereof.

Embodiment 46. The immunomodulatory molecule of embodiment 45, wherein the second binding domain is a variant of the immunosuppressive cytokine, wherein the variant of the immunosuppressive cytokine has increased or decreased binding affinity to the second target molecule compared to the immunosuppressive cytokine.

Embodiment 47. The immunomodulatory molecule of embodiment 45 or 46, wherein the second binding domain is IL-10 or variant thereof.

Embodiment 48. The immunomodulatory molecule of embodiment 45 or 46, wherein the second binding domain is TGF-β or variant thereof.

Embodiment 49. The immunomodulatory molecule of embodiment 43 or 44, wherein the second binding domain is an agonist antibody or antigen-binding fragment thereof.

Embodiment 50. The immunomodulatory molecule of any one of embodiments 43-49, wherein the first binding domain is an antagonist antibody or antigen-binding fragment thereof.

Embodiment 51. The immunomodulatory molecule of any one of embodiments 43-49, wherein the first binding domain is antagonist ligand or variant thereof.

Embodiment 52. The immunomodulatory molecule of embodiment 51, wherein the first binding domain is a variant of an antagonist ligand, and wherein the variant of the antagonist ligand has increased or decreased binding affinity to the first target molecule compared to the antagonist ligand.

Embodiment 53. The immunomodulatory molecule of any one of embodiments 1-5, wherein the first target molecule and/or the second target molecule is an inhibitory immune cell surface receptor.

Embodiment 54. The immunomodulatory molecule of embodiment 53, wherein the inhibitory immune cell surface receptor is selected from the group consisting of CD5, NKG2A, NKG2B, KLRG1, FCRL4, Siglec2, CD72, CD244, GP49B, Lair-1, PirB, PECAM-1, CD200R, ILT2, and KIR2DL.

Embodiment 55. The immunomodulatory molecule of embodiment 53 or 54, wherein the second binding domain is an agonist antibody or antigen-binding fragment thereof.

Embodiment 56. The immunomodulatory molecule of embodiment 53 or 54, wherein the second binding domain is an agonist ligand or variant thereof.

Embodiment 57. The immunomodulatory molecule of embodiment 56, wherein the second binding domain is a variant of an agonist ligand, wherein the variant of the agonist ligand has increased or decreased binding affinity to the second target molecule compared to the agonist ligand.

Embodiment 58. The immunomodulatory molecule of any one of embodiments 53-57, wherein the first binding domain is an antagonist antibody or antigen-binding fragment thereof.

Embodiment 59. The immunomodulatory molecule of any one of embodiments 53-57, wherein the first binding domain is an antagonist ligand or variant thereof.

Embodiment 60. The immunomodulatory molecule of embodiment 59, wherein the first binding domain is a variant of an antagonist ligand, and wherein the variant of the antagonist ligand has increased or decreased binding affinity to the first target molecule compared to the antagonist ligand.

Embodiment 61. The immunomodulatory molecule of any one of embodiments 1-5, wherein the first binding domain is IL-12 or variant thereof, and wherein the second binding domain is an agonist antibody or antigen-binding fragment thereof specifically recognizing PD-1.

Embodiment 62. The immunomodulatory molecule of any one of embodiments 1-5, wherein the first binding domain is IL-12 or variant thereof, and wherein the second binding domain is PD-L1 or variant thereof.

Embodiment 63. The immunomodulatory molecule of embodiment 62, wherein the second binding domain is a variant of PD-L1, and wherein the variant of PD-L1 has increased or decreased binding affinity to the second target molecule compared to PD-L1.

Embodiment 64. The immunomodulatory molecule of any one of embodiments 1-5, wherein the first binding domain is IL-12 or variant thereof, and wherein the second binding domain is PD-L2 or variant thereof.

Embodiment 65. The immunomodulatory molecule of embodiment 64, wherein the second binding domain is a variant of PD-L2, and wherein the variant of PD-L2 has increased or decreased binding affinity to the second target molecule compared to PD-L2.

Embodiment 66. The immunomodulatory molecule of any one of embodiments 61-65, wherein the first binding domain is a variant of IL-12, and wherein the variant of IL-12 has increased or decreased binding affinity to the first target molecule compared to IL-12.

Embodiment 67. The immunomodulatory molecule of any one of embodiments 1-5, wherein the first binding domain is IL-2 or variant thereof, and wherein the second binding domain is an agonist antibody or antigen-binding fragment thereof specifically recognizing PD-1.

Embodiment 68. The immunomodulatory molecule of any one of embodiments 1-5, wherein the first binding domain is IL-2 or variant thereof, and wherein the second binding domain is PD-L1 or variant thereof.

Embodiment 69. The immunomodulatory molecule of embodiment 68, wherein the second binding domain is a variant of PD-L1, and wherein the variant of PD-L1 has increased or decreased binding affinity to the second target molecule compared to PD-L1.

Embodiment 70. The immunomodulatory molecule of any one of embodiments 1-5, wherein the first binding domain is IL-2 or variant thereof, and wherein the second binding domain is PD-L2 or variant thereof.

Embodiment 71. The immunomodulatory molecule of embodiment 70, wherein the second binding domain is a variant of PD-L2, and wherein the variant of PD-L2 has increased or decreased binding affinity to the second target molecule compared to PD-L2.

Embodiment 72. The immunomodulatory molecule of any one of embodiments 67-71, wherein the first binding domain is a variant of IL-2, and wherein the variant of IL-2 has increased or decreased binding affinity to the first target molecule compared to IL-2.

Embodiment 73. The immunomodulatory molecule of any one of embodiments 1-72, wherein the immunomodulatory molecule comprises: i) an antigen-binding protein comprising an antigen-binding polypeptide; and ii) the first binding domain, wherein the antigen-binding polypeptide comprises from N-terminus to C-terminus: the second binding domain or portion thereof, a hinge region, and an Fc domain subunit or portion thereof, and wherein the first binding domain is positioned at the hinge region.

Embodiment 74. The immunomodulatory molecule of embodiment 73, wherein in the presence of binding of the second binding domain to the second target molecule, the activity of the first binding domain increases at least about 20% compared to that in the absence of binding of the second binding domain to the second target molecule.

Embodiment 75. The immunomodulatory molecule of embodiment 73 or 74, wherein in the absence of binding of the second binding domain to the second target molecule, the activity of the first binding domain positioned at the hinge region is no more than about 70% of that of a corresponding first binding domain in a free state.

Embodiment 76. The immunomodulatory molecule of any one of embodiments 73-75, wherein the antigen-binding protein comprises two antigen-binding polypeptides each comprising a hinge region, and wherein only one antigen-binding polypeptide comprises the first binding domain positioned at the hinge region.

Embodiment 77. The immunomodulatory molecule of any one of embodiments 73-75, wherein the antigen-binding protein comprises two antigen-binding polypeptides each comprising a hinge region, and wherein each antigen-binding polypeptide comprises a first binding domain positioned at the hinge region.

Embodiment 78. The immunomodulatory molecule of any one of embodiments 73-77, wherein the immunomodulatory molecule comprises two or more first binding domains, wherein the two or more first binding domains are positioned in tandem at the hinge region of the antigen-binding polypeptide.

Embodiment 79. The immunomodulatory molecule of any one of embodiments 73-78, wherein the first binding domain is an immunostimulatory cytokine or variant thereof.

Embodiment 80. The immunomodulatory molecule of embodiment 79, wherein the immunostimulatory cytokine is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF.

Embodiment 81. The immunomodulatory molecule of embodiment 79 or 80, wherein the first binding domain is an immunostimulatory cytokine variant, and wherein the activity of the immunostimulatory cytokine variant in a free state is no more than about 80% of that of a corresponding wildtype immunostimulatory cytokine in a free state.

Embodiment 82. The immunomodulatory molecule of any one of embodiments 79-81, wherein the immunostimulatory cytokine or variant thereof is a monomeric immunostimulatory cytokine or variant thereof.

Embodiment 83. The immunomodulatory molecule of any one of embodiments 79-81, wherein the immunostimulatory cytokine or variant thereof is a dimeric immunostimulatory cytokine or variant thereof.

Embodiment 84. The immunomodulatory molecule of embodiment 83, wherein both subunits of the dimeric immunostimulatory cytokine or variant thereof are positioned in tandem at the hinge region of the antigen-binding polypeptide.

Embodiment 85. The immunomodulatory molecule of embodiment 83, wherein the antigen-binding protein comprises two antigen-binding polypeptides each comprising a hinge region, wherein one subunit of the dimeric immunostimulatory cytokine or variant thereof is positioned at the hinge region of one antigen-binding polypeptide, and wherein the other subunit of the dimeric immunostimulatory cytokine or variant thereof is positioned at the hinge region of the other antigen-binding polypeptide.

Embodiment 86. The immunomodulatory molecule of any one of embodiments 79-82, wherein the immunostimulatory cytokine or variant thereof is IL-2 or variant thereof.

Embodiment 87. The immunomodulatory molecule of embodiment 86, wherein the IL-2 variant comprises one or more mutations at a position selected from the group consisting of F24, K35, R38, F42, K43, E61, and P65 relative to a wildtype IL-2.

Embodiment 88. The immunomodulatory molecule of embodiment 86 or 87, wherein the IL-2 variant comprises one or more mutations selected from the group consisting of F24A, R38D, K43E, E61R, and P65L relative to a wildtype IL-2.

Embodiment 89. The immunomodulatory molecule of any one of embodiments 86-88, wherein the IL-2 variant comprises an R38D/K43E/E61R mutation relative to a wildtype IL-2.

Embodiment 90. The immunomodulatory molecule of any one of embodiments 79-81 and 83-85, wherein the immunostimulatory cytokine or variant thereof is IL-12 or variant thereof.

Embodiment 91. The immunomodulatory molecule of embodiment 90, wherein the IL-12 variant comprises one or more mutations within the p40 subunit at a position selected from the group consisting of E45, Q56, V57, K58, E59, F60, G61, D62, A63, G64, Q65, and C177 relative to a wildtype p40 subunit.

Embodiment 92. The immunomodulatory molecule of embodiment 90 or 91, wherein the IL-12 variant comprises one or more mutations within the p40 subunit selected from the group consisting of Q56A, V57A, K58A, E59A, F60A, G61A, D62A, A63S, G64A, and Q65A relative to a wildtype p40 subunit.

Embodiment 93. The immunomodulatory molecule of any one of embodiments 90-92, wherein the IL-12 variant comprises an E59A/F60A mutation within the p40 subunit relative to a wildtype p40 subunit.

Embodiment 94. The immunomodulatory molecule of any one of embodiments 90-92, wherein the IL-12 variant comprises an F60A mutation within the p40 subunit relative to a wildtype p40 subunit.

Embodiment 95. The immunomodulatory molecule of any one of embodiments 90-94, wherein the p40 subunit and the p35 subunit of the IL-12 or variant thereof are connected by a linker.

Embodiment 96. The immunomodulatory molecule of any one of embodiments 77-95, wherein the two or more first binding domains are the same.

Embodiment 97. The immunomodulatory molecule of any one of embodiments 77-95, wherein the two or more first binding domains are different.

Embodiment 98. The immunomodulatory molecule of any one of embodiments 73-97, wherein the second binding domain is an agonist ligand or variant thereof of an inhibitory checkpoint molecule.

Embodiment 99. The immunomodulatory molecule of embodiment 98, wherein the inhibitory checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, Siglec7, Siglec9, and VISTA.

Embodiment 100. The immunomodulatory molecule of embodiment 98 or 99, wherein the second binding domain is PD-L1 or variant thereof.

Embodiment 101. The immunomodulatory molecule of embodiment 100, wherein the PD-L1 variant has increased binding affinity to PD-1 compared to a wildtype PD-L1.

Embodiment 102. The immunomodulatory molecule of embodiment 100 or 101, wherein the PD-L1 variant comprises one or more mutations at a position selected from the group consisting of 154, Y56, E58, R113, M115, S117, and G119 relative to a wildtype PD-L1.

Embodiment 103. The immunomodulatory molecule of any one of embodiments 100-102, wherein the PD-L1 variant comprises one or more mutations selected from the group consisting of I54Q, Y56F, E58M, R 113T, M115L, S117A, and G119K relative to a wildtype PD-L1.

Embodiment 104. The immunomodulatory molecule of any one of embodiments 100-103, wherein the PD-L1 variant comprises an I54Q/Y56F/E58M/R113T/M115L/S117A/G119K mutation relative to a wildtype PD-L1.

Embodiment 105. The immunomodulatory molecule of embodiment 98 or 99, wherein the second binding domain is PD-L2 or variant thereof.

Embodiment 106. The immunomodulatory molecule of embodiment 105, wherein the PD-L2 variant has increased binding affinity to PD-1 compared to a wildtype PD-L2.

Embodiment 107. The immunomodulatory molecule of any one of embodiments 73-97, wherein the second binding domain is an agonist antibody or antigen-binding fragment thereof of an inhibitory checkpoint molecule.

Embodiment 108. The immunomodulatory molecule of embodiment 107, wherein the inhibitory checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, Siglec7, Siglec9, and VISTA.

Embodiment 109. The immunomodulatory molecule of embodiment 107 or 108, wherein the agonist antibody or antigen-binding fragment thereof specifically recognizes PD-1 (“anti-PD-1 agonist antibody or antigen-binding fragment thereof”).

Embodiment 110. The immunomodulatory molecule of any one of embodiments 107-109, wherein the agonist antibody or antigen-binding fragment thereof is a Fab.

Embodiment 111. The immunomodulatory molecule of any one of embodiments 107-109, wherein the agonist antibody or antigen-binding fragment thereof is an scFv.

Embodiment 112. The immunomodulatory molecule of any one of embodiments 73-111, wherein the antigen-binding protein comprises two or more second binding domains.

Embodiment 113. The immunomodulatory molecule of embodiment 112, wherein the two or more second binding domains or portions thereof are positioned in tandem at the N-terminus of the antigen-binding polypeptide.

Embodiment 114. The immunomodulatory molecule of embodiment 112 or 113, wherein the antigen-binding protein comprises two antigen-binding polypeptides each comprising a hinge region, and wherein only one antigen-binding polypeptide comprises the two or more second binding domains or portions thereof positioned in tandem at the N-terminus of the antigen-binding polypeptide.

Embodiment 115. The immunomodulatory molecule of embodiment 112 or 113, wherein the antigen-binding protein comprises two antigen-binding polypeptides each comprising a hinge region, and wherein each antigen-binding polypeptide comprises one or more second binding domains or portions thereof at the N-terminus of each antigen-binding polypeptide.

Embodiment 116. The immunomodulatory molecule of any one of embodiments 73-114, wherein the antigen-binding protein comprises two antigen-binding polypeptides each comprising a hinge region, wherein the first antigen-binding polypeptide comprises one or more second binding domains or portions thereof at the N-terminus of the first antigen-binding polypeptide, wherein the second antigen-binding polypeptide comprises a third binding domain or portion thereof at the N-terminus of the second antigen-binding polypeptide, and wherein the third binding domain specifically recognizing a third target molecule.

Embodiment 117. The immunomodulatory molecule of embodiment 116, wherein the third binding domain and the second binding domain are the same.

Embodiment 118. The immunomodulatory molecule of embodiment 116, wherein the third binding domain and the second binding domain are different.

Embodiment 119. The immunomodulatory molecule of any one of embodiments 116-118, wherein the third target molecule and the second target molecule are the same.

Embodiment 120. The immunomodulatory molecule of embodiment 116 or 118, wherein the third target molecule and the second target molecule are different.

Embodiment 121. The immunomodulatory molecule of any one of embodiments 73-120, comprising: i) a first antigen-binding polypeptide comprising from N-terminus to C-terminus: a first PD-L2 or PD-L1 or variant thereof, a second PD-L2 or PD-L1 or variant thereof, a p35 subunit and a p40 subunit of an IL-12 or variant thereof positioned in tandem at a first hinge region, and a first subunit of an Fc domain or portion thereof; ii) a second antigen-binding polypeptide comprising from N-terminus to C-terminus: a VH, an optional CH1, a second hinge region, and a second subunit of the Fc domain or portion thereof; and iii) a third antigen-binding polypeptide comprising from N-terminus to C-terminus: a VL, and an optional CL; wherein the VH and the VL and optionally the CH1 and the CL form a third binding domain specifically recognizing a third target molecule.

Embodiment 122. The immunomodulatory molecule of embodiment 121, wherein the third binding domain is an agonist antigen-binding fragment specifically recognizing PD-1.

Embodiment 123. The immunomodulatory molecule of any one of embodiments 73-120, comprising: i) a first antigen-binding polypeptide comprising from N-terminus to C-terminus: a first VH, an optional first CH1, a p35 subunit and a p40 subunit of an IL-12 or variant thereof positioned in tandem at a first hinge region, and a first subunit of an Fc domain or portion thereof; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule.

Embodiment 124. The immunomodulatory molecule of embodiment 123, wherein the third binding domain is an agonist antigen-binding fragment specifically recognizing PD-1.

Embodiment 125. The immunomodulatory molecule of any one of embodiments 73-120, comprising: i) a first antigen-binding polypeptide comprising from N-terminus to C-terminus: a first PD-L2 or PD-L1 or variant thereof, a p35 subunit and a p40 subunit of an IL-12 or variant thereof positioned in tandem at a first hinge region, and a first subunit of an Fc domain or portion thereof; and ii) a second antigen-binding polypeptide comprising from N-terminus to C-terminus: a second PD-L2 or PD-L1 or variant thereof, a second hinge region, and a second subunit of an Fc domain or portion thereof.

Embodiment 126. The immunomodulatory molecule of any one of embodiments 73-120, comprising: i) a first antigen-binding polypeptide comprising from N-terminus to C-terminus: a first PD-L2 or PD-L1 or variant thereof, a second PD-L2 or PD-L1 or variant thereof, a p35 subunit and a p40 subunit of an IL-12 or variant thereof positioned in tandem at a first hinge region, and a first subunit of an Fc domain or portion thereof; and ii) a second antigen-binding polypeptide comprising from N-terminus to C-terminus: a third PD-L2 or PD-L1 or variant thereof, a fourth PD-L2 or PD-L1 or variant thereof, a second hinge region, and a second subunit of the Fc domain or portion thereof.

Embodiment 127. The immunomodulatory molecule of any one of embodiments 73-120, comprising: i) a first antigen-binding polypeptide comprising from N-terminus to C-terminus: a first PD-L2 or PD-L1 or variant thereof, a p35 subunit of an IL-12 or variant thereof positioned at a first hinge region, and a first subunit of an Fc domain or portion thereof; and ii) a second antigen-binding polypeptide comprising from N-terminus to C-terminus: a second PD-L2 or PD-L1 or variant thereof, a p40 subunit of an IL-12 or variant thereof positioned at a second hinge region, and a second subunit of the Fc domain or portion thereof.

Embodiment 128. The immunomodulatory molecule of any one of embodiments 73-120, comprising: i) a first antigen-binding polypeptide comprising from N-terminus to C-terminus: a p35 subunit or a p40 subunit of an IL-12 or variant thereof positioned at a first hinge region, and a first subunit of an Fc domain or portion thereof; and ii) a second antigen-binding polypeptide comprising from N-terminus to C-terminus: a first PD-L2 or PD-L1 or variant thereof, a second PD-L2 or PD-L1 or variant thereof, a p40 subunit or a p35 subunit of an IL-12 or variant thereof positioned at a second hinge region, and a second subunit of the Fc domain or portion thereof.

Embodiment 129. The immunomodulatory molecule of any one of embodiments 73-120, comprising: i) a first antigen-binding polypeptide comprising from N-terminus to C-terminus: a first VH, an optional first CH1, a p35 subunit or a p40 subunit of an IL-12 or variant thereof positioned at a first hinge region, and a first subunit of an Fc domain or portion thereof; ii) a second antigen-binding polypeptide comprising from N-terminus to C-terminus: a second VH, an optional second CH1, a p40 subunit or a p35 subunit of an IL-12 or variant thereof positioned at a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule.

Embodiment 130. The immunomodulatory molecule of embodiment 129, wherein the third binding domain is an agonist antigen-binding fragment specifically recognizing PD-1.

Embodiment 131. The immunomodulatory molecule of any one of embodiments 1-72, wherein the immunomodulatory molecule comprises an antigen-binding protein comprising an antigen-binding polypeptide, wherein the antigen-binding polypeptide comprises from N′ to C′: the first binding domain or portion thereof, the second binding domain or portion thereof, an optional hinge region, and an Fc domain subunit or portion thereof.

Embodiment 132. The immunomodulatory molecule of embodiment 131, wherein the second binding domain is an agonist Fab or an agonist scFv that specifically recognizes an inhibitory checkpoint molecule.

Embodiment 133. The immunomodulatory molecule of embodiment 131, wherein the second binding domain is an agonist ligand or variant thereof of an inhibitory checkpoint molecule.

Embodiment 134. The immunomodulatory molecule of embodiment 133, wherein the second binding domain is PD-L1 or PD-L2 or variant thereof.

Embodiment 135. The immunomodulatory molecule of any one of embodiments 131-134, wherein the first binding domain is an immunostimulatory cytokine or variant thereof.

Embodiment 136. The immunomodulatory molecule of embodiment 135, wherein the immunostimulatory cytokine or variant thereof is IL-2 or IL-12 or variant thereof.

Embodiment 137. The immunomodulatory molecule of any one of embodiments 131-136, wherein the antigen-binding protein comprises two antigen-binding polypeptides each comprising a hinge region, wherein the first antigen-binding polypeptide comprises from N′ to C′: the first binding domain or portion thereof, the second binding domain or portion thereof, a first hinge region, and a first subunit of an Fc domain or portion thereof; wherein the second antigen-binding polypeptide comprises from N′ to C′: a third binding domain or portion thereof, a second hinge region, and a second subunit of the Fc domain or portion thereof; and wherein the third binding domain specifically recognizing a third target molecule.

Embodiment 138. The immunomodulatory molecule of embodiment 137, wherein the third binding domain and the second binding domain are the same.

Embodiment 139. The immunomodulatory molecule of embodiment 137, wherein the third binding domain and the second binding domain are different.

Embodiment 140. The immunomodulatory molecule of any one of embodiments 137-139, wherein the third target molecule and the second target molecule are the same.

Embodiment 141. The immunomodulatory molecule of embodiment 137 or 139, wherein the third target molecule and the second target molecule are different.

Embodiment 142. The immunomodulatory molecule of any one of embodiments 131-141, comprising: i) a first antigen-binding polypeptide comprising from N-terminus to C-terminus: a p35 subunit and a p40 subunit of an IL-12 or variant thereof fused in tandem, a first VH, an optional first CH1, a first hinge region, and a first subunit of an Fc domain or portion thereof; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule.

Embodiment 143. The immunomodulatory molecule of embodiment 142, wherein the third binding domain is an agonist antigen-binding fragment specifically recognizing PD-1.

Embodiment 144. The immunomodulatory molecule of any one of embodiments 131-141, comprising: i) a first antigen-binding polypeptide comprising from N-terminus to C-terminus: a p35 subunit and a p40 subunit of an IL-12 or variant thereof fused in tandem, a first PD-L2 or PD-L1 or variant thereof, a second PD-L2 or PD-L1 or variant thereof, a first hinge region, and a first subunit of an Fc domain or portion thereof; and ii) a second antigen-binding polypeptide comprising from N-terminus to C-terminus: a third PD-L2 or PD-L1 or variant thereof, a fourth PD-L2 or PD-L1 or variant thereof, a second hinge region, and a second subunit of the Fc domain or portion thereof.

Embodiment 145. The immunomodulatory molecule of any one of embodiments 131-141, comprising: i) a first antigen-binding polypeptide comprising from N-terminus to C-terminus: a p35 subunit and a p40 subunit of an IL-12 or variant thereof fused in tandem, a first PD-L2 or PD-L1 or variant thereof, a second PD-L2 or PD-L1 or variant thereof, a first hinge region, and a first subunit of an Fc domain or portion thereof; ii) a second antigen-binding polypeptide comprising from N-terminus to C-terminus: a VH, an optional CH1, a second hinge region, and a second subunit of the Fc domain or portion thereof; and iii) a third antigen-binding polypeptide comprising from N-terminus to C-terminus: a VL, and an optional CL, wherein the VH and the VL and optionally the CH1 and the CL form a third binding domain specifically recognizing a third target molecule.

Embodiment 146. The immunomodulatory molecule of embodiment 145, wherein the third binding domain is an agonist antigen-binding fragment specifically recognizing PD-1.

Embodiment 147. The immunomodulatory molecule of any one of embodiments 131-141, comprising: i) a first antigen-binding polypeptide comprising from N-terminus to C-terminus: a p35 subunit and a p40 subunit of an IL-12 or variant thereof fused in tandem, a VH, an optional CH1, a first hinge region, and a first subunit of an Fc domain or portion thereof; ii) a second antigen-binding polypeptide comprising from N-terminus to C-terminus: a first PD-L2 or PD-L1 or variant thereof, a second PD-L2 or PD-L1 or variant thereof, a second hinge region, and a second subunit of the Fc domain or portion thereof; and iii) a third antigen-binding polypeptide comprising from N-terminus to C-terminus: a VL, and an optional CL, wherein the VH and the VL and optionally the CH1 and the CL form the second binding domain which is an agonist antigen-binding fragment specifically recognizing PD-1.

Embodiment 148. The immunomodulatory molecule of any one of embodiments 1-72, comprising: i) a first antigen-binding polypeptide comprising from N-terminus to C-terminus: a first VH, an optional first CH1, a first hinge region, and a first subunit of an Fc domain or portion thereof; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a p35 subunit and a p40 subunit of an IL-12 or variant thereof fused in tandem, a first VL, and an optional first CL; and iv) a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule.

Embodiment 149. The immunomodulatory molecule of embodiment 148, wherein the third binding domain is an agonist antigen-binding fragment specifically recognizing PD-1.

Embodiment 150. The immunomodulatory molecule of any one of embodiments 1-72, comprising: i) a first antigen-binding polypeptide comprising from N-terminus to C-terminus: a VH, an optional CH1, a first hinge region, and a first subunit of an Fc domain or portion thereof; ii) a second antigen-binding polypeptide comprising from N-terminus to C-terminus: a first PD-L2 or PD-L1 or variant thereof, a second PD-L2 or PD-L1 or variant thereof, a second hinge region, and a second subunit of the Fc domain or portion thereof; and iii) a third antigen-binding polypeptide comprising from N-terminus to C-terminus: a p35 subunit and a p40 subunit of an IL-12 or variant thereof fused in tandem, a VL, and an optional CL, wherein the VH and the VL and optionally the CH1 and the CL form the second binding domain which is an agonist antigen-binding fragment specifically recognizing PD-1.

Embodiment 151. The immunomodulatory molecule of any one of embodiments 1-72, wherein the immunomodulatory molecule comprises an antigen-binding protein comprising a first antigen-binding polypeptide and a second antigen-binding polypeptide, wherein the first antigen-binding polypeptide comprises from N-terminus to C-terminus: the second antigen binding domain or portion thereof, a first hinge domain, and a first subunit of an Fc domain or portion thereof; wherein the second antigen-binding polypeptide comprises from N-terminus to C-terminus: the first antigen binding domain or portion thereof, a second hinge domain, and a second subunit of the Fc domain or portion thereof.

Embodiment 152. The immunomodulatory molecule of embodiment 151, wherein the second binding domain is an agonist Fab or an agonist scFv that specifically recognizes an inhibitory checkpoint molecule.

Embodiment 153. The immunomodulatory molecule of embodiment 151, wherein the second binding domain is an agonist ligand or variant thereof of an inhibitory checkpoint molecule.

Embodiment 154. The immunomodulatory molecule of embodiment 153, wherein the second binding domain is PD-L1 or PD-L2 or variant thereof.

Embodiment 155. The immunomodulatory molecule of any one of embodiments 151-154, wherein the first binding domain is an immunostimulatory cytokine or variant thereof.

Embodiment 156. The immunomodulatory molecule of embodiment 155, wherein the immunostimulatory cytokine or variant thereof is IL-2 or IL-12 or variant thereof.

Embodiment 157. The immunomodulatory molecule of any one of embodiments 151-156, comprising: i) a first antigen-binding polypeptide comprising from N-terminus to C-terminus: a VH, an optional CH1, a first hinge region, and a first subunit of an Fc domain or portion thereof; ii) a second antigen-binding polypeptide comprising from N-terminus to C-terminus: a p35 subunit and a p40 subunit of an IL-12 or variant thereof fused in tandem, a second hinge region, and a second subunit of the Fc domain or portion thereof; and iii) a third antigen-binding polypeptide comprising from N-terminus to C-terminus: a VL, and an optional CL, wherein the VH and the VL and optionally the CH1 and the CL form the second binding domain which is an agonist antigen-binding fragment specifically recognizing PD-1.

Embodiment 158. The immunomodulatory molecule of any one of embodiments 151-156, comprising: i) a first antigen-binding polypeptide comprising from N-terminus to C-terminus: a first PD-L2 or PD-L1 or variant thereof, a second PD-L2 or PD-L1 or variant thereof, a first hinge region, and a first subunit of an Fc domain or portion thereof; and ii) a second antigen-binding polypeptide comprising from N-terminus to C-terminus: a p35 subunit and a p40 subunit of an IL-12 or variant thereof fused in tandem, a second hinge region, and a second subunit of the Fc domain or portion thereof.

Embodiment 159. The immunomodulatory molecule of any one of embodiments 1-72, wherein the immunomodulatory molecule comprises an antigen-binding protein comprising an antigen-binding polypeptide, wherein the antigen-binding polypeptide comprises from N-terminus to C-terminus: the second binding domain or portion thereof, an optional hinge region, an Fc domain subunit or portion thereof, and the first binding domain or portion thereof.

Embodiment 160. The immunomodulatory molecule of embodiment 159, wherein the second binding domain is an agonist Fab or an agonist scFv that specifically recognizes an inhibitory checkpoint molecule.

Embodiment 161. The immunomodulatory molecule of embodiment 159, wherein the second binding domain is an agonist ligand or variant thereof of an inhibitory checkpoint molecule.

Embodiment 162. The immunomodulatory molecule of embodiment 161, wherein the second binding domain is PD-L1 or PD-L2 or variant thereof.

Embodiment 163. The immunomodulatory molecule of any one of embodiments 159-162, wherein the first binding domain is an immunostimulatory cytokine or variant thereof.

Embodiment 164. The immunomodulatory molecule of embodiment 163, wherein the immunostimulatory cytokine or variant thereof is IL-2 or IL-12 or variant thereof.

Embodiment 165. The immunomodulatory molecule of embodiment 163 or 164, wherein the immunostimulatory cytokine or variant thereof is a monomeric immunostimulatory cytokine or variant thereof.

Embodiment 166. The immunomodulatory molecule of embodiment 163 or 164, wherein the immunostimulatory cytokine or variant thereof is a dimeric immunostimulatory cytokine or variant thereof.

Embodiment 167. The immunomodulatory molecule of embodiment 166, wherein both subunits of the dimeric immunostimulatory cytokine or variant thereof are positioned in tandem at the C-terminus of the antigen-binding polypeptide.

Embodiment 168. The immunomodulatory molecule of embodiment 166, wherein the antigen-binding protein comprises two antigen-binding polypeptides each comprising a hinge region and an Fc domain subunit or portion thereof, wherein one subunit of the dimeric immunostimulatory cytokine or variant thereof is fused to the C-terminus of the Fc domain subunit or portion thereof of one antigen-binding polypeptide, and wherein the other subunit of the dimeric immunostimulatory cytokine or variant thereof is fused to the C-terminus of the Fc domain subunit or portion thereof of the other antigen-binding polypeptide.

Embodiment 169. The immunomodulatory molecule of embodiment 168, wherein the antigen-binding polypeptide not comprising the second binding domain or portion thereof comprises from N-terminus to C-terminus: a third binding domain or portion thereof specifically recognizing a third target molecule, the hinge region, the subunit of the Fc domain or portion thereof, and the subunit of the dimeric immunostimulatory cytokine or variant thereof.

Embodiment 170. The immunomodulatory molecule of any one of embodiments 159-168, wherein the antigen-binding protein comprises a first antigen-binding polypeptide and a second antigen-binding polypeptide, wherein the first antigen-binding polypeptide comprises from N-terminus to C-terminus: the second binding domain or portion thereof, a first hinge region, a first subunit of an Fc domain or portion thereof, and the first binding domain or portion thereof; wherein the second antigen-binding polypeptide comprises from N′ to C′: a third binding domain or portion thereof specifically recognizing a third target molecule, a second hinge region, and a second subunit of the Fc domain or portion thereof.

Embodiment 171. The immunomodulatory molecule of embodiment 169 or 170, wherein the third binding domain and the second binding domain are the same.

Embodiment 172. The immunomodulatory molecule of embodiment 169 or 170, wherein the third binding domain and the second binding domain are different.

Embodiment 173. The immunomodulatory molecule of any one of embodiments 169-172, wherein the third target molecule and the second target molecule are the same.

Embodiment 174. The immunomodulatory molecule of any one of embodiments 169, 170, and 172, wherein the third target molecule and the second target molecule are different.

Embodiment 175. The immunomodulatory molecule of any one of embodiments 159-174, comprising: i) a first antigen-binding polypeptide comprising from N-terminus to C-terminus: a first PD-L2 or PD-L1 or variant thereof, a first hinge region, a first subunit of an Fc domain or portion thereof, and a p35 subunit and a p40 subunit of an IL-12 or variant thereof fused in tandem; and ii) a second antigen-binding polypeptide comprising from N-terminus to C-terminus: a second PD-L2 or PD-L1 or variant thereof, a second hinge region, and a second subunit of the Fc domain or portion thereof.

Embodiment 176. The immunomodulatory molecule of any one of embodiments 159-174, comprising: i) a first antigen-binding polypeptide comprising from N-terminus to C-terminus: a first VH, an optional first CH1, a first hinge region, a first subunit of an Fc domain or portion thereof, and a p35 subunit and a p40 subunit of an IL-12 or variant thereof fused in tandem; ii) a second antigen-binding polypeptide comprising from N-terminus to C-terminus: a second VH, an optional second CH1, a second hinge region, and a second subunit of the Fc domain or portion thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule.

Embodiment 177. The immunomodulatory molecule of embodiment 176, wherein the third binding domain is an agonist antigen-binding fragment specifically recognizing PD-1.

Embodiment 178. The immunomodulatory molecule of any one of embodiments 159-174, comprising: i) a first antigen-binding polypeptide comprising from N-terminus to C-terminus: a VH, an optional CH1, a first hinge region, a first subunit of an Fc domain or portion thereof, and a p35 subunit and a p40 subunit of an IL-12 or variant thereof fused in tandem; ii) a second antigen-binding polypeptide comprising from N-terminus to C-terminus: a first PD-L2 or PD-L1 or variant thereof, a second PD-L2 or PD-L1 or variant thereof, a second hinge region, and a second subunit of the Fc domain or portion thereof; and iii) a third antigen-binding polypeptide comprising from N-terminus to C-terminus: a VL, and an optional CL, wherein the VH and the VL and optionally the CH1 and the CL form the second binding domain which is an agonist antigen-binding fragment specifically recognizing PD-1.

Embodiment 179. The immunomodulatory molecule of any one of embodiments 159-174, comprising: i) a first antigen-binding polypeptide comprising from N-terminus to C-terminus: a first PD-L2 or PD-L1 or variant thereof, a first hinge region, a first subunit of an Fc domain or portion thereof, and a p35 subunit or a p40 subunit of an IL-12 or variant thereof; and ii) a second antigen-binding polypeptide comprising from N-terminus to C-terminus: a second PD-L2 or PD-L1 or variant thereof, a second hinge region, and a second subunit of the Fc domain or portion thereof, and a p40 subunit or a p35 subunit of an IL-12 or variant thereof.

Embodiment 180. The immunomodulatory molecule of any one of embodiments 159-174, comprising: i) a first antigen-binding polypeptide comprising from N-terminus to C-terminus: a first VH, an optional first CH1, a first hinge region, a first subunit of an Fc domain or portion thereof, and a p35 subunit or a p40 subunit of an IL-12 or variant thereof; ii) a second antigen-binding polypeptide comprising from N-terminus to C-terminus: a second VH, an optional second CH1, a second hinge region, a second subunit of the Fc domain or portion thereof, and a p40 subunit or a p35 subunit of an IL-12 or variant thereof; iii) a third antigen-binding polypeptide comprising from N-terminus to C-terminus: a first VL, and an optional first CL; and iv) a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule.

Embodiment 181. The immunomodulatory molecule of embodiment 180, wherein the third binding domain is an agonist antigen-binding fragment specifically recognizing PD-1.

Embodiment 182. The immunomodulatory molecule of any one of embodiments 1-72, wherein the immunomodulatory molecule comprises an antigen-binding protein comprising a first antigen-binding polypeptide and a second antigen-binding polypeptide, wherein the first antigen-binding polypeptide comprises from N-terminus to C-terminus: a VH, a CH1, an optional hinge region, an Fc domain subunit or portion thereof; wherein the second antigen-binding polypeptide comprises from N-terminus to C-terminus: a VL, a CL, and the first binding domain or portion thereof; and wherein the VH and the VL and optionally the CH1 and the CL form the second binding domain.

Embodiment 183. The immunomodulatory molecule of embodiment 182, wherein the first antigen-binding polypeptide comprises from N-terminus to C-terminus: a VH, a CH1, a first hinge region, a first subunit of an Fc domain or portion thereof; wherein the antigen-binding protein further comprises a third antigen-binding polypeptide comprising from N-terminus to C-terminus: a third binding domain or portion thereof specifically recognizing a third target molecule, a second hinge region, and a second subunit of the Fc domain or portion thereof.

Embodiment 184. The immunomodulatory molecule of embodiment 183, wherein the third binding domain and the second binding domain are the same.

Embodiment 185. The immunomodulatory molecule of embodiment 183, wherein the third binding domain and the second binding domain are different.

Embodiment 186. The immunomodulatory molecule of any one of embodiments 183-185, wherein the third target molecule and the second target molecule are the same.

Embodiment 187. The immunomodulatory molecule of embodiment 183 or 185, wherein the third target molecule and the second target molecule are different.

Embodiment 188. The immunomodulatory molecule of any one of embodiments 183-187, wherein the immunomodulatory molecule comprises an antigen-binding protein comprising four antigen-binding polypeptides, wherein the first antigen-binding polypeptide comprises from N-terminus to C-terminus: a first VH, a first CH1, a first hinge region, a first subunit of an Fc domain or portion thereof; wherein the second antigen-binding polypeptide comprises from N-terminus to C-terminus: a first VL, a first CL, and the first binding domain or portion thereof; wherein the third antigen-binding polypeptide comprising from N-terminus to C-terminus: a second VH, a second CH1, a second hinge region, and a second subunit of the Fc domain or portion thereof; wherein the fourth antigen-binding polypeptide comprises from N-terminus to C-terminus: a second VL, and a second CL; wherein the first VH and the first VL and the first CH1 and the first CL form the second binding domain; and wherein the second VH and the second VL and the second CH1 and the second CL form a third binding domain specifically recognizing a third target molecule.

Embodiment 189. The immunomodulatory molecule of any one of embodiments 182-188, wherein the first binding domain is an immunostimulatory cytokine or variant thereof.

Embodiment 190. The immunomodulatory molecule of embodiment 189, wherein the immunostimulatory cytokine or variant thereof is IL-2 or IL-12 or variant thereof.

Embodiment 191. The immunomodulatory molecule of embodiment 189 or 190, wherein the immunostimulatory cytokine or variant thereof is a monomeric immunostimulatory cytokine or variant thereof.

Embodiment 192. The immunomodulatory molecule of embodiment 189 or 190, wherein the immunostimulatory cytokine or variant thereof is a dimeric immunostimulatory cytokine or variant thereof.

Embodiment 193. The immunomodulatory molecule of embodiment 192, wherein both subunits of the dimeric immunostimulatory cytokine or variant thereof are positioned in tandem at the C-terminus of the second antigen-binding polypeptide and/or the fourth antigen-binding polypeptide.

Embodiment 194. The immunomodulatory molecule of embodiment 192, wherein one subunit of the dimeric immunostimulatory cytokine or variant thereof is fused to the C-terminus of the first CL of the second antigen-binding polypeptide, and wherein the other subunit of the dimeric immunostimulatory cytokine or variant thereof is fused to the second CL of the fourth antigen-binding polypeptide.

Embodiment 195. The immunomodulatory molecule of any one of embodiments 182-194, comprising: i) a first antigen-binding polypeptide comprising from N-terminus to C-terminus: a first VH, a first CH1, a first hinge region, and a first subunit of an Fc domain or portion thereof; ii) a second antigen-binding polypeptide comprising from N-terminus to C-terminus: a first VL, a first CL, and a p35 subunit and a p40 subunit of an IL-12 or variant thereof fused in tandem; iii) a third antigen-binding polypeptide comprising from N-terminus to C-terminus: a second VH, a second CH1, a second hinge region, and a second subunit of the Fc domain or portion thereof; and iv) a fourth antigen-binding polypeptide comprising from N-terminus to C-terminus: a second VL, a second CL, and a p35 subunit and a p40 subunit of an IL-12 or variant thereof fused in tandem; wherein the first VH and the first VL and the first CH1 and the first CL form the second binding domain which is an agonist antigen-binding fragment specifically recognizing PD-1, and wherein the second VH and the second VL and the second CH1 and the second CL form a third binding domain specifically recognizing a third target molecule.

Embodiment 196. The immunomodulatory molecule of embodiment 195, wherein the third binding domain is an agonist antigen-binding fragment specifically recognizing PD-1.

Embodiment 197. An isolated nucleic acid encoding the immunomodulatory molecule of any one of embodiments 1-196.

Embodiment 198. A vector comprising the nucleic acid of embodiment 197.

Embodiment 199. An isolated host cell comprising the nucleic acid of embodiment 197 or the vector of embodiment 198.

Embodiment 200. The host cell of embodiment 199, which is a Chinese hamster ovary (CHO) cell.

Embodiment 201. A method of producing an immunomodulatory molecule, comprising: (a) culturing a host cell comprising the nucleic acid of embodiment 197 or the vector of embodiment 198, or a host cell of embodiment 199 or 200, under a condition effective to express the encoded immunomodulatory molecule; and (b) obtaining the expressed immunomodulatory molecule from said host cell.

Embodiment 202. A pharmaceutical composition comprising the immunomodulatory molecule of any one of embodiments 1-196, and optionally a pharmaceutical acceptable carrier.

Embodiment 203. A method of treating a disease or disorder in an individual, comprising administering to the individual an effective amount of the immunomodulatory molecule of any one of embodiments 1-196, or the pharmaceutical composition of embodiment 202.

Embodiment 204. The method of embodiment 203, wherein the immunomodulatory molecule or pharmaceutical composition is administered intravenously or subcutaneously.

Embodiment 205. The method of embodiment 203 or 204, wherein the immunomodulatory molecule or pharmaceutical composition is administered in an amount of about 1 μg/kg to about 10 mg/kg.

Embodiment 206. The method of any one of embodiments 203-205, wherein the immunomodulatory molecule or pharmaceutical composition is administered once every three weeks.

Embodiment 207. The method of any one of embodiments 203-206, wherein the disease or disorder is a cancer.

Embodiment 208. The method of embodiment 207, wherein the cancer is selected from the group consisting of lung cancer, liver cancer, renal cancer, colorectal cancer, ovarian cancer, breast cancer, pancreatic cancer, gastric carcinoma, bile duct cancer, squamous cell carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphoma, myeloma, mycoses fungoides, and merkel cell cancer.

Embodiment 209. The method of any one of embodiments 203-206, wherein the disease or disorder is an infection, an autoimmune disease, an allergy, a graft rejection, or a graft-versus-host disease (GvHD).

### EXAMPLES

The examples below are intended to be purely exemplary of the invention and should therefore not be considered to limit the invention in any way. The following examples and detailed description are offered by way of illustration and not by way of limitation.

### Example 1: In Vitro Analysis of IL-12 Biological Activities in IL-12/PD-L1-Fc, PD-L1-Fc/IL-12, IL-12/Anti-PD-1, CTLA-4-Fc/IL-12, and IL-12/CTLA-4-Fc Immunomodulatory Molecules

### Construction of IL-12/PD-L1-Fc, PD-L1-Fc/IL-12, IL-12/Anti-PD-1, CTLA-4-Fc/IL-12, and IL-12/CTLA-4-Fc Immunomodulatory Molecules

IL-12 is a heterodimeric cytokine composed of covalently linked p35 and p40 subunits. IL-12 variants comprising amino acid substitution in the p40 subunit were constructed by replacing amino acids from position 56 to 65 of the p40 subunit with Alanine or Serine (see Table 1), and a single chain IL-12 variant was made, from N′ to C′: p40 variant subunit-linker (SEQ ID NO: 228)-p35 wildtype subunit (SEQ ID NO: 61). A single chain “wildtype” IL-12 was also constructed as a control (SEQ ID NO: 67), from N′ to C′: p40 wildtype subunit (SEQ ID NO: 62)-linker (SEQ ID NO: 228)-p35 wildtype subunit, referred to as “WT” in Table 1. The linker can also be changed to SEQ ID NO: 226, and the single chain “wildtype” IL-12 can also comprise SEQ ID NO: 253.

### IL-12/Anti-PD-1 (Hinge) Immunomodulatory Molecules

An anti-human PD-1 antibody comprising nivolumab (Opdivo®) VH (SEQ ID NO: 48) and VL (SEQ ID NO: 49) sequences was used as the parental full-length antibody, comprising two light chains each comprising the amino acid sequence of SEQ ID NO: 50. To construct heterodimer, one heavy chain comprises a hinge region comprising SEQ ID NO: 78, and an Fc domain subunit comprising SEQ ID NO: 97; the other heavy chain comprises a hinge region comprising SEQ ID NO: 77, and an Fc domain subunit comprising SEQ ID NO; 98. Various single chain IL-12 variants (or single chain “wildtype” IL-12 control) were positioned within the hinge region of a heavy chain of the anti-PD-1 antibody (see FIG. 1C for exemplary structure, anti-PD-1 nivolumab is antagonist antibody), to construct IL-12/anti-PD-1 immunomodulatory molecule “Fab-IL-12-Fc-PD-1 Ab.” For example, Fab-IL-12(E59A/F60A)-Fc-PD-1 Ab immunomodulatory molecule (or “IL-12(E59A/F60A)/anti-PD-1 immunomodulatory molecule”, “IW-#48”, or “construct #48”) comprising a single-chain IL-12 variant IL-12B (p40 E59A/F60A)-linker-IL-12A (wt p35) positioned at the hinge region comprises two light chains each comprising the amino acid sequence of SEQ ID NO: 50, one heavy chain comprising the amino acid sequence of SEQ ID NO: 21, and one heavy chain with the single-chain IL-12(E59A/F60A) variant (SEQ ID NO: 68) positioned at the hinge region comprising the amino acid sequence of SEQ ID NO: 22. IL-12(G64A)/anti-PD-1 immunomodulatory molecule (“construct #47”, or “IW-#47”) comprising a single-chain IL-12 variant IL-12B (p40 G64A)-linker-IL-12A (wt p35) positioned at the hinge region comprises two light chains each comprising the amino acid sequence of SEQ ID NO: 50, one heavy chain comprising the amino acid sequence of SEQ ID NO: 21, and one heavy chain with the single-chain IL-12(G64A) variant (SEQ ID NO: 70) positioned at the hinge region. IL-12(E59A)/anti-PD-1 immunomodulatory molecule comprising a single-chain IL-12 variant IL-12B (p40 E59A)-linker-IL-12A (wt p35) positioned at the hinge region comprises two light chains each comprising the amino acid sequence of SEQ ID NO: 50, one heavy chain comprising the amino acid sequence of SEQ ID NO: 21, and one heavy chain with the single-chain IL-12(E59A) variant (SEQ ID NO: 69) positioned at the hinge region. IL-12(F60A)/anti-PD-1 immunomodulatory molecule (“construct #46”, or “IW-#46”) comprising a single-chain IL-12 variant IL-12B (p40 F60A)-linker-IL-12A (wt p35) positioned at the hinge region comprises two light chains each comprising the amino acid sequence of SEQ ID NO: 50, one heavy chain comprising the amino acid sequence of SEQ ID NO: 21, and one heavy chain with the single-chain IL-12(F60A) variant (SEQ ID NO: 71) positioned at the hinge region comprising the amino acid sequence of SEQ ID NO: 23. The heavy chain comprising the amino acid sequence of SEQ ID NO: 21 can also be replaced with a heavy chain comprising the amino acid sequence of SEQ ID NO: 51. The linker within the single-chain IL-12 variant (e.g., single-chain IL-12(E59A/F60A) variant) can also be changed to SEQ ID NO: 246, and the single-chain IL-12(E59A/F60A) variant can comprise SEQ ID NO: 254.

### IL-12/PD-L1-Fc (Hinge) and PD-L1-Fc/IL-12 (C-Terminal) Immunomodulatory Molecules

A PD-L1-hinge-Fc fusion protein (two PD-L1 extracellular domain-hinge-Fc polypeptides) was used as parental antigen-binding protein to construct immunomodulatory molecules that bind to PD-1. To construct heterodimeric PD-L1-hinge-Fc fusion protein, one PD-L1-hinge-Fc fusion polypeptide comprises a hinge region comprising SEQ ID NO: 88, and an Fc domain subunit comprising SEQ ID NO: 97; the other PD-L2-Fc fusion polypeptide comprises a hinge region comprising SEQ ID NO: 87, and an Fc domain subunit comprising SEQ ID NO: 98. Single chain IL-12 variant described above was either positioned at the hinge region of one PD-L1-hinge-Fc polypeptide (hereinafter referred to as “IL-12/PD-L1-Fc immunomodulatory molecule”), or fused to the C-terminus of one PD-L1-hinge-Fc polypeptide (hereinafter referred to as “PD-L1-Fc/IL-12 immunomodulatory molecule”).

For example, IL-12(E59A/F60A)/PD-L1(wt)-Fc immunomodulatory molecule comprises one IL-12 fusion polypeptide from N′ to C′: PD-L1(wt) extracellular domain (SEQ ID NO: 121)-GGGGSGGG linker (SEQ ID NO: 244)-single chain IL-12(E59A/F60A) variant-GGGGSGGG linker (SEQ ID NO: 244)-hinge (SEQ ID NO: 88)-Fc domain subunit (SEQ ID NO: 97); and one pairing polypeptide from N′ to C′: PD-L1(wt) extracellular domain (SEQ ID NO: 121)-GGGGSGGG linker (SEQ ID NO: 244)-hinge (SEQ ID NO: 87)-Fc domain subunit (SEQ ID NO: 98). IL-12(E59A/F60A)/PD-L1(mut)-Fc immunomodulatory molecule comprises one IL-12 fusion polypeptide from N′ to C′: PD-L1(mut) extracellular domain (e.g., SEQ ID NO: 129)-GGGGSGGG linker (SEQ ID NO: 244)-single chain IL-12(E59A/F60A) variant-GGGGSGGG linker (SEQ ID NO: 244)-hinge (SEQ ID NO: 88)-Fc domain subunit (SEQ ID NO: 97); and one pairing polypeptide from N′ to C′: PD-L1(mut) extracellular domain-GGGGSGGG linker (SEQ ID NO: 244)-hinge (SEQ ID NO: 87)-Fc domain subunit (SEQ ID NO: 98). PD-L1(wt)-Fc/IL-12(E59A/F60A) (C-terminal) immunomodulatory molecule comprises one IL-12 fusion polypeptide comprising from N′ to C′: PD-L1 (wt) extracellular domain (SEQ ID NO: 121)-GSG linker (SEQ ID NO: 203)-hinge (SEQ ID NO: 88)-Fc domain subunit (SEQ ID NO: 97)-GGGGSGGGGSGGGGS linker (SEQ ID NO: 229)-single chain IL-12(E59A/F60A) variant (SEQ ID NO: 68 or 254); and one pairing polypeptide comprising from N′ to C′: PD-L1 (wt) extracellular domain (SEQ ID NO: 121)-GSG linker (SEQ ID NO: 203)-hinge (SEQ ID NO: 87)-Fc domain subunit (SEQ ID NO: 98)). The linkers can be changed to other linkers (e.g., GSG linker; SEQ ID NO: 203) or can be optional.

IL-12/CTLA-4-Fc (hinge) and CTLA-4-Fc/IL-12 (C-terminal) immunomodulatory molecules were similarly constructed. See Table 2 for sequences.

Nucleic acids encoding various formats of IL-12/PD-L1-Fc, IL-12/anti-PD-1, and IL-12/CTLA-4-Fc immunomodulatory molecules were chemically synthesized (see Table 2 for amino acid sequences of each polypeptide chain), cloned into a lentiviral vector, and transfected into CHO cells for expression. Expressed immunomodulatory molecules were collected from supernatant, purified by protein A chromatography, and verified on SDS-PAGE for purity.

### IL-12 Signal Transduction Assay

HEK-Blue™ IL-12 Cells (InvivoGen Cat. #hkb-il12) and HEK-PD-1-IL-12 cells (generated in-house by overexpressing human PD-1 in HEK-Blue™ IL-12 Cells using a lentiviral vector) were used to assess IL-12 signal activation activity of the various IL-12 immunomodulatory molecules comprising different IL-12 moieties, following the InvivoGen user manual (InvivoGen Cat. #hkb-il12), hereinafter also referred to as “HEK-IL-12 reporter assay” or “HEK-PD-1-IL-12 reporter assay.” HEK-Blue™ IL-12 reporter cells and HEK-PD-1-IL-12 reporter cells stably express the human IL-12 receptor complex consisting of the IL-12 receptor 01 (IL-12Rβ1) and IL-12Rβ2, along with the human STAT4 gene to obtain a fully functional IL-12 signaling pathway (TyK2/JAK2/STAT4). In addition, these reporter cells also carry a STAT4-inducible SEAP reporter gene. Upon IL-12 stimulation, HEK-Blue™ IL-12 reporter cells and HEK-PD-1-IL-12 reporter cells trigger the activation of STAT4 and the subsequent secretion of SEAP, the levels of which can be monitored using QUANTT-Blue™ (InvivoGen Cat. #rep-qbs) colorimetric enzyme assay for alkaline phosphatase activity.

Briefly, HEK-Blue™ IL-12 cells were added to various IL-12 containing immunomodulatory molecules in each plate well (or recombinant human IL-12 (rIL-12) in a well as positive control), and incubated at 37° C. in a C02 incubator for 20-24 hours or overnight. After incubation, supernatant was transferred to fresh plate wells, added QUANTI-Blue™ solution, and incubated at 37° C. incubator for 30 minutes-3 hours. Then SEAP levels were determined using a spectrophotometer at 620-655 nm. The activity of recombinant human IL-12 (positive control) in activating IL-12 signaling pathway was measured as 10 unit/ng and served as a reference. Percent IL-12 signal transduction for various IL-12 immunomodulatory molecules was calculated by dividing the immunomodulatory molecules readout by the recombinant human IL-12 readout.

In HEK-PD-1-IL-12 reporter assay, IL-12/anti-PD-1 immunomodulatory molecules were only able to bind to HEK-IL-12 cells via binding PD-1 or IL-12 moiety/IL-12 receptor interaction. Positioning IL-12 comprising wildtype p40 subunit at the hinge region of the anti-PD-1 antibody (“IL-12(WT)/anti-PD-1”) reduced IL-12 activity to 50.0%, in the absence of PD-1 binding. As can be seen from Table 1, positions 59 and 60 of p40 subunit are crucial for IL-12 biological activity. IL-12/anti-PD-1 immunomodulatory molecule comprising E59A/F60A double mutations in the IL-12 p40 subunit (“IL-12(E59A/F60A)/anti-PD-1”, “IW-#48”) showed almost completely aborted IL-12 activity as measured by IL-12 signal transduction (0.1%).

In HEK-PD-1-IL-12 reporter assay, IL-12/anti-PD-1 immunomodulatory molecules were able to bind to HEK-PD-1-IL-12 cells via both IL-12 moiety/IL-12 receptor interaction, and anti-PD-1 antigen-binding fragment/PD-1 interaction. As can be seen from Table 1, the biological activity of all IL-12 variants (and “WT” IL-12) in IL-12/anti-PD-1 immunomodulatory molecules increased with the presence of PD-1 binding. Especially, the IL-12 activity of IL-12(E59A/F60A)/anti-PD-1 immunomodulatory molecule (IW-#48) was rescued by PD-1/anti-PD-1 antibody binding to 5.9%, which was 59-fold of that in the absence of PD-1/anti-PD-1 antibody binding (0.1%).

E59A/F60A double mutations in the IL-12 p40 subunit demonstrated superior effect compared to other mutations in IL-12 p40 subunit. By positioning this IL-12(E59A/F60A) variant at the hinge region of a heavy chain of the anti-PD-1 full-length antibody, the obtained IL-12(E59A/F60A)/anti-PD-1 immunomodulatory molecule (IW-#48) only exhibited IL-12 biological activity in the presence of target antigen (PD-1)-antibody binding, but not in the absence of target antigen (PD-1)-antibody binding, demonstrating targeted specificity.

TABLE 1
IL-12 biological activity of IL-12/anti-PD-1 immunomodulatory
molecules comprising different IL-12 moieties

| | rIL-12 | “WT” | Q56A | V57A | K58A | E59A | F60A |
|---|---|---|---|---|---|---|---|
| HEK-IL-12 cells | 100.0% | 50.0% | 55.0% | 48.0% | 39.0% | 10.0% | 9.0% |
| HEK-PD-1-IL-12 cells | 100.0% | 120.0% | 150.0% | 91.0% | 98.0% | 45.0% | 56.0% |

| | G61A | D62A | A63S | G64A | Q65A | E59A/F60A |
|---|---|---|---|---|---|---|
| HEK-IL-12 cells | 56.0% | 57.0% | 62.0% | 69.0% | 62.0% | 0.1% |
| HEK-PD-1-IL-12 cells | 130.0% | 93.0% | 89.0% | 150.0% | 130.0% | 5.9% |

In HEK-PD-1-IL-12 reporter assay, IL-12 immunomodulatory molecules were only able to bind to HEK-IL-12 cells via binding PD-1 or IL-12 moiety/IL-12 receptor interaction. The CTLA-4/IL-12 immunomodulatory molecules were unable to bind to the PD-1+ HEK cells, which do not express receptors such as CD80 or CD86. The biological activity of IL-12 in the absence of receptor (CD80 or CD86)-ligand (CTLA-4) binding from these CLTA-4 immunomodulatory molecules (Table 2; SEQ ID NOs: 1-5) was non-existent: 0.2-2.3%. Notably, the single mutant IL-12 (F60A) at the C-terminus without the receptor (CD80 or CD86)-ligand (CTLA-4) binding shown some activity (2.3%) while the single mutant IL-12 (F60A) at the hinge completely lost any activity (0.2%). The wild-type PD-L1 ligand (wt extracellular domain SEQ ID NO: 121) binding to PD-1 is low affinity (K_{d} of ˜8.2 μM). The mutations in PD-L1 ligand (I54Q/Y56F/E58M/R113T/M115L/S117A/G119K; mutant 8 extracellular domain SEQ ID NO: 129) increased binding affinity by about 200 fold (K_{d} of ˜0.041 μM). The wild-type PD-L1 ligand binding to PD-1 from these wild type constructs (Table 2; SEQ ID NOs: 6-10) rescued the biological activity of mutant IL-12 (greater 100%) with the exception of the double mutant IL12 (E59A/F60A) located in the hinge region (8.2%). In contrast, the high affinity PD-L1 ligand from these mutant PD-L1 constructs (Table 2; SEQ ID NOs: 11-15) rescued all of the biological activity of mutant IL-12 including in the mutant IL-12(E59A/F60A) location at the hinge (125.6%). These data suggested that the activity of the mutant IL-12 (E59A/F60A) could be rescued by increasing the binding affinity of the PD-L1 ligand in the same construct. The rescue of biological activity depends on the affinity of PD-L1 ligand to PD-1. Furthermore, the activity of IL-12 in the presence of PD-L1/PD-1 and IL-12/IL-12R binding was greater than the positive control (rIL-12 alone). This indicates that the presence of a second domain binding to the target cell (e.g., PD-L1/PD-1 binding on T cell) facilitates IL-12 immunomodulatory molecules binding to the same target cell (e.g., IL-12/IL-12R binding on T cell).

TABLE 2
IL-12 biological activity of various IL-12 immunomodulatory molecules

| Second | Second | First binding | | IL-12B | Biological | |
|---|---|---|---|---|---|---|
| binding | binding | domain (IL- | | subunit | activity of | Construct |
| domain | domain | 12) location | Structure | mutations | IL-12 | sequences |
| CTLA-4 | Wildtype | C-terminus of | FIG. 1I | E59A/F60A | 0.6% | 1^{st} polypeptide chain SEQ |
| extracellular | | heavy chain | | | | ID NO: 1; 2^{nd} polypeptide |
| domain | | | | | | chain SEQ ID NO: 2 |
| (SEQ ID | | | | F60A | 2.3% | 1^{st} polypeptide chain SEQ |
| NO: 141) | | | | | | ID NO: 1; 2^{nd} polypeptide |
| | | | | | | chain SEQ ID NO: 3 |
| | | Hinge | FIG. 1G | E59A/F60A | 0.3% | 1^{st} polypeptide chain SEQ |
| | | | | | | ID NO: 1; 2^{nd} polypeptide |
| | | | | | | chain SEQ ID NO: 4 |
| | | | | F60A | 0.2% | 1^{st} polypeptide chain SEQ |
| | | | | | | ID NO: 1; 2^{nd} polypeptide |
| | | | | | | chain SEQ ID NO: 5 |
| PD-L1 | I54Q/Y56F/ | C-terminus | FIG. 1I | E59A/F60A | 189.0% | 1^{st} polypeptide chain SEQ |
| extracellular | E58M/ | of Fc | | | | ID NO: 11; 2^{nd} polypeptide |
| domain | R113T/ | | | | | chain SEQ ID NO: 12 |
| (high affinity) | M115L/ | | | F60A | 234.1% | 1^{st} polypeptide chain SEQ |
| (SEQ ID | S117A/ | | | | | ID NO: 11; 2^{nd} polypeptide |
| NO: 129) | G119K | | | | | chain SEQ ID NO: 13 |
| | | Hinge | FIG. 1G | E59A/F60A | 125.6% | 1^{st} polypeptide chain SEQ |
| | | | | | | ID NO: 11; 2^{nd} polypeptide |
| | | | | | | chain SEQ ID NO: 14 |
| | | | | F60A | 233.5% | 1^{st} polypeptide chain SEQ |
| | | | | | | ID NO: 11; 2^{nd} polypeptide |
| | | | | | | chain SEQ ID NO: 15 |
| PD-L1 | Wildtype | C-terminus of | FIG. 1I | E59A/F60A | 200.2% | 1^{st} polypeptide chain SEQ |
| extracellular | | Fc | | | | ID NO: 6; 2^{nd} polypeptide |
| domain | | | | | | chain SEQ ID NO: 7 |
| (low affinity) | | | | F60A | 227.5% | 1^{st} polypeptide chain SEQ |
| (SEQ ID | | | | | | ID NO: 6; 2^{nd} polypeptide |
| NO: 121) | | | | | | chain SEQ ID NO: 8 |
| | | Hinge | FIG. 1G | E59A/F60A | 8.2% | 1^{st} polypeptide chain SEQ |
| | | | | | | ID NO: 6; 2^{nd} polypeptide |
| | | | | | | chain SEQ ID NO: 9 |
| | | | | F60A | 221.0% | 1^{st} polypeptide chain SEQ |
| | | | | | | ID NO: 6; 2^{nd} polypeptide |
| | | | | | | chain SEQ ID NO: 10 |

### Example 2: In Vivo Efficacy of IL-12/PD-L2-Fc and PD-L2-Fc/IL-12 Immunomodulatory Molecules in Established CT26 Syngeneic Tumor Mice Model

### Construction of IL-12/PD-L2-Fc (Hinge) and PD-L2-Fc/IL-12 (C-Terminal) Immunomodulatory Molecules

A PD-L2-hinge-Fc fusion protein (two PD-L2-hinge-Fc polypeptides each comprising SEQ ID NO: I11) was used as parental antigen-binding protein to construct immunomodulatory molecules that bind to PD-1. To construct heterodimeric PD-L2-hinge-Fc fusion protein, one PD-L2-hinge-Fc fusion polypeptide comprises a hinge region comprising SEQ ID NO: 88, and an Fc domain subunit comprising SEQ ID NO: 97, the other PD-L2-Fc fusion polypeptide comprises a hinge region comprising SEQ ID NO: 87, and an Fc domain subunit comprising SEQ ID NO: 98. Single chain IL-12 variant described above was either positioned at the hinge region of one PD-L2-hinge-Fc polypeptide (hereinafter referred to as “IL-12/PD-L2-Fc immunomodulatory molecule”), or fused to the C-terminus of one PD-L2-hinge-Fc polypeptide (hereinafter referred to as “PD-L2-Fc/IL-12 immunomodulatory molecule”). For example, IL-12(E59A/F60A)/PD-L2-Fc immunomodulatory molecule (“construct #29” or “IW-#29”) comprises one IL-12 fusion polypeptide comprising SEQ ID NO: 17 (from N′ to C′: PD-L2 extracellular domain (SEQ ID NO: 106)-GSG linker (SEQ ID NO: 203)-single chain IL-12(E59A/F60A) variant (SEQ ID NO: 68)-hinge (SEQ ID NO: 88)-Fc domain subunit (SEQ ID NO: 97)); and one pairing polypeptide comprising SEQ ID NO: 16 (from N′ to C′: PD-L2 extracellular domain (SEQ ID NO: 106)-GSG linker (SEQ ID NO: 203)-hinge (SEQ ID NO: 87)-Fc domain subunit (SEQ ID NO: 98)). The single chain IL-12(E59A/F60A) variant within IL-12(E59A/F60A)/PD-L2-Fc immunomodulatory molecule was replaced with either single-chain IL-12 (F60A) variant (SEQ ID NO: 71) or single-chain IL-12 (G64A) variant (SEQ ID NO: 70) to construct IL-12(F60A)/PD-L2-Fc immunomodulatory molecule (“construct #30” or “IW-#30”) and IL-12(G64A)/PD-L2-Fc immunomodulatory molecule, respectively. For example, IL-12(F60A)/PD-L2-Fc immunomodulatory molecule comprises one IL-12 fusion polypeptide comprising SEQ ID NO: 18 or 142 (from N′ to C′: PD-L2 extracellular domain (SEQ ID NO: 106)-GSG linker (SEQ ID NO: 203)-single chain IL-12(F60A) variant (SEQ ID NO: 71)-hinge (SEQ ID NO: 88)-Fc domain subunit (SEQ ID NO: 97)); and one pairing polypeptide comprising SEQ ID NO: 16 or 115 (from N′ to C′: PD-L2 extracellular domain (SEQ ID NO: 106)-GSG linker (SEQ ID NO: 203)-hinge (SEQ ID NO: 87)-Fc domain subunit (SEQ ID NO: 98)). PD-L2-Fc/IL-12(F60A) immunomodulatory molecule (“construct #34” or “IW-#34”) comprises one IL-12 fusion polypeptide comprising SEQ ID NO: 20 or 143 (from N′ to C′: PD-L2 extracellular domain (SEQ ID NO: 106)-GSG linker (SEQ ID NO: 203)-hinge (SEQ ID NO: 88)-Fc domain subunit (SEQ ID NO: 97)-GGGGSGGGGSGGGGS linker (SEQ ID NO: 229)-single chain IL-12(F60A) variant (SEQ ID NO: 71)); and one pairing polypeptide comprising SEQ ID NO: 16 or 115 (from N′ to C′: PD-L2 extracellular domain (SEQ ID NO: 106)-GSG linker (SEQ ID NO: 203)-hinge (SEQ ID NO: 87)-Fc domain subunit (SEQ ID NO: 98)). The linker within the single-chain IL-12 variant (e.g., single-chain IL-12(E59A/F60A) variant) can also be changed to SEQ ID NO: 246, for example, the single-chain IL-12(E59A/F60A) variant can comprise SEQ ID NO: 254.

Nucleic acids encoding immunomodulatory molecules were chemically synthesized, cloned into a lentiviral vector, and transfected into CHO cells for expression. Expressed immunomodulatory molecules were collected from supernatant, purified by protein A chromatography, and verified on SDS-PAGE for purity.

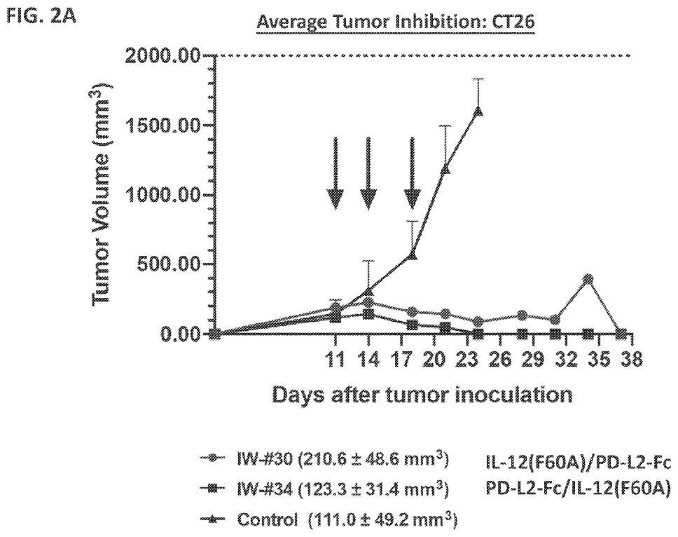

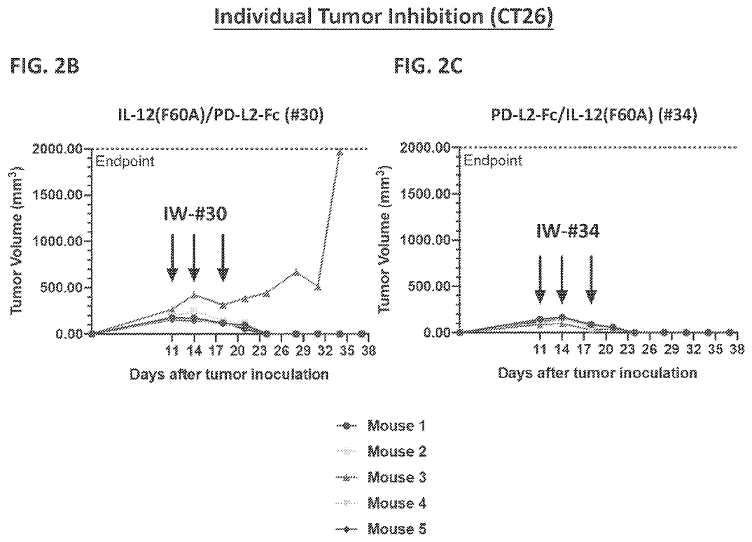

Mice (˜20g body weight) were inoculated with 0.25×10⁶ CT26 murine colon cancer cells. Eleven days after tumor inoculation, tumor size was measured to be about 100-200 mm³. After measuring tumor size, mice were injected with 200 μg (10 mg/kg) IL-12(F60A)/PD-L2-Fc, hinge (IW-#30) immunomodulatory molecule (see FIG. 1G for structure (first polypeptide chain SEQ ID NO: 16 or 115, second polypeptide chain SEQ ID NO: 18 or 142), wherein the cytokine is a variant IL-12 F60A), 200 μg (10 mg/kg) IL-12(F60A)/PD-L2-Fc, C-terminus of HC (IW-#34) immunomodulatory molecule (see FIG. 11 for structure (first polypeptide chain SEQ ID NO: 16 or 115, second polypeptide chain SEQ ID NO: 20 or 143), wherein the cytokine is a variant IL-12 F60A; also referred to herein as PD-L2-Fc/IL-12(F60A)), or PBS (negative control). Each group had five mice. A total of three injections were given on days 11, 14, and 18 post-inoculation (indicated by black arrows in FIGS. 2A-2C). Tumor size was measured every 3 days since the first injection. The average initial (before injection) tumor volume plus or minus one standard deviation is given in parenthesis in the figure legend. Mice were sacrificed once tumor size reached over 2000 mm³. FIG. 2A depicts the average tumor volume (±standard deviation) in each treatment group. Individual mice plots for each group were also provided in FIG. 2B showing IL-12(F60A)/PD-L2-Fc, hinge (IW-#30) immunomodulatory molecule and FIG. 2C showing IL-12(F60A)/PD-L2-Fc, C-terminus of HC (IW-#34) immunomodulatory molecule. IL-12(F60A)/PD-L2-Fc, hinge (IW-#30) immunomodulatory molecule cured 4/5 mice (80% cure rate) and IL-12(F60A)/PD-L2-Fc, C-terminus of HC (IW-#34) immunomodulatory molecule cured 5/5 mice (100% cure rate). Among cured mice, the tumor inhibition efficacy of both IL-12/PD-L2-Fc immunomodulatory molecules was similar.

CT26 mice model is highly responsive to current immunotherapies, including anti-PD-1, anti-CTLA-4, and combination treatment with anti-PD-1 and anti-CTLA-4 antibodies. Data here showed that both IL-12/PD-L2-Fc immunomodulatory molecules were capable of regressing CT26 tumors in 80-100% of mice, demonstrating promising in vivo efficacy.

### Example 3: IL-12/PD-L2-Fc and PD-L2-Fc/IL-12 Immunomodulatory Molecules are Capable of Induce Specific, Anti-Tumor Memory in Cured CT26 Syngeneic Tumor Mice Models

To investigate if immunomodulatory molecules described herein can function as cancer vaccine, or prevent cancer recurrence, a tumor re-challenge was conducted on all cured mice from Example 2. Thirty days after the final immunomodulatory molecule injection, cured CT26 mice were inoculated with 0.25-106 CT26 murine colon cancer cells on the right flank and 0.25′106 EMT6 murine breast cancer cells on the left flank (as control). Tumor sizes were recorded every 4 days following re-challenge tumor inoculation. Mice were sacrificed once tumor size reached over 1000 mm³.

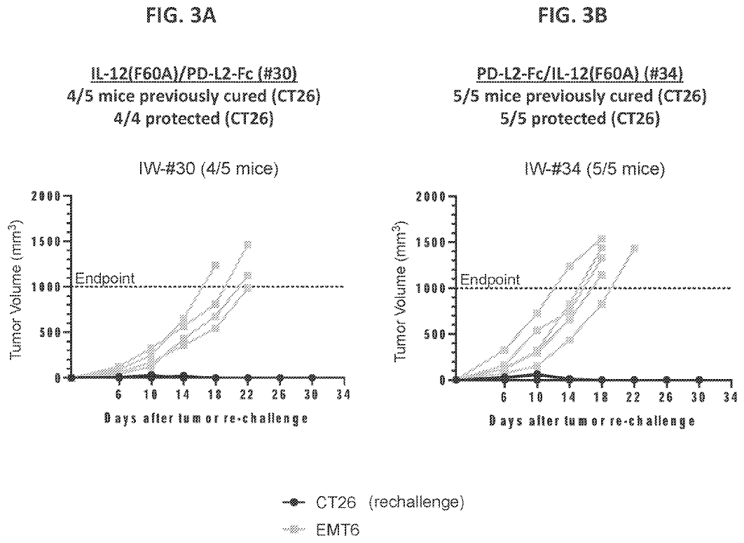

As shown in FIGS. 3A-3B, all cured mice previously treated with immunomodulatory molecules against CD26 tumor were protected from CT26 tumor re-challenge but not from EMT6. IL-12(F60A)/PD-L2-Fc immunomodulatory molecule (IW-#30; hinge) and PD-L2-Fc/IL-12(F60A) immunomodulatory molecule (IW-#34; C-terminus of HC) demonstrated similar protection efficacy against CT26 tumor re-challenge. These results indicate successful generation of anti-CT26 tumor memory, suggesting that immunomodulatory molecules described herein, such as both PD-L2-Fc/IL-12(F60A) immunomodulatory molecule (IW-#34; C-terminus of HC) and IL-12(F60A)/PD-L2-Fc immunomodulatory molecule (IW-#30; hinge), can serve as a cancer vaccine (e.g., against CT26 colon cancer) in mice, and/or can prevent cancer recurrence, capable of inducing induce specific, anti-tumor memory.

### Example 4: IL-12/PD-L2-Fc Immunomodulatory Molecules are Capable of Regressing Very Large CT26 Tumors (>250 mm³) or Late-Stage CT26 Tumors

Successful therapies for late-stage cancers remain a huge unmet clinical need. To study if immunomodulatory molecules described herein are effective in treating late-stage cancers, mice were inoculated with cancer cells, tumor was allowed to grow to bigger than 250 mm³, which is considered untreatable with immunotherapy in mice. Such murine tumor volume may mimic tumor burdens in advanced, late-stage human cancer patients.

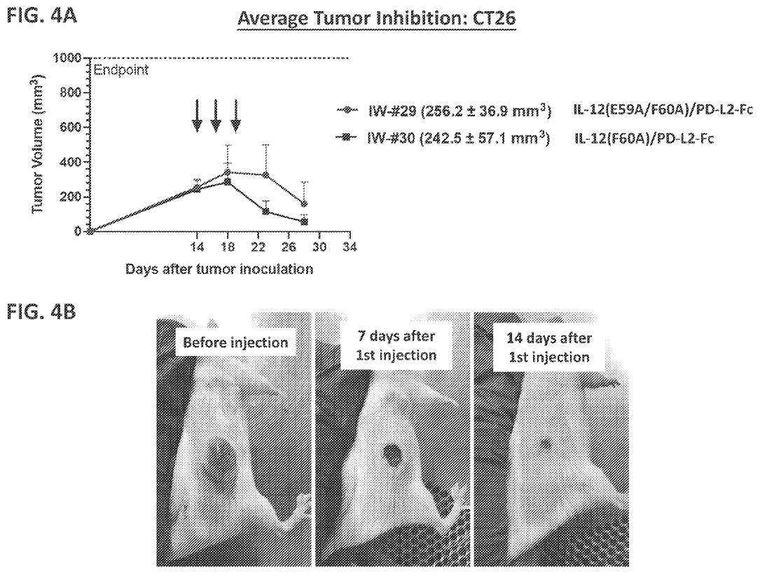

Briefly, mice (˜20g body weight) were inoculated with 0.25×10⁶ CT26 murine colon cancer cells. Fourteen days after tumor inoculation, tumor size was measured to be greater than 250 mm³. The average initial tumor volume plus or minus one standard deviation is given in parenthesis in the figure legend of FIG. 4A. After measuring tumor size, mice were injected with 200 μg (10 mg/kg) IL-12(E59A/F60A)/PD-L2-Fc, hinge (constructed in Example 2, IW-#29) immunomodulatory molecule (see FIG. 1G for structure (first polypeptide chain SEQ ID NO: 16, second polypeptide chain SEQ ID NO: 17), wherein the cytokine is a variant IL-12 E59A/F60A), 200 μg (10 mg/kg) IL-12(F60A)/PD-L2-Fc, hinge (constructed in Example 2, IW-#30) immunomodulatory molecule (see FIG. 1G for structure (first polypeptide chain SEQ ID NO: 16, second polypeptide chain SEQ ID NO: 18), wherein the cytokine is a variant IL-12 F60A). Each group had seven mice. A total of three injections were given on days 14, 17, and 21 post-inoculation (indicated by black arrows). Tumor size was measured every 4 days since the first injection. Mice were sacrificed once tumor size reached over 1000 mm³. FIG. 4A depicts the average tumor volume in each treatment group.

As seen in FIG. 4A, the tumor regression efficacy difference seen between IL-12(E59A/F60A)/PD-L2-Fc, hinge (IW-#29) immunomodulatory molecule and IL-12(F60A)/PD-L2-Fc, hinge (IW-#30) immunomodulatory molecule was likely due to lower potency (e.g., receptor binding and/or signal activation ability) of double mutation IL-12(E59A/F60A) compared to single mutation IL-12(F60A). Such efficacy difference may be compensated by higher dosing per injection (e.g., 20 mg/kg vs. 10 mg/kg), or more injections (e.g., increase from 3 to 5 injections) of IL-12(E59A/F60A)-based immunomodulatory molecules.

FIG. 4B depicts pictures of a mouse over the course of treatment with IL-12(F60A)/PD-L2-Fc, hinge (IW-#30) immunomodulatory molecule. The initial tumor volume was 290.4 mm³. The structural integrity of the tumor quickly degraded within a week following first injection and formed a scab. Two weeks after initial injection, the tumor has completely regressed.

Immunotherapy (monotherapy or combination) usually fails to respond in syngeneic tumor volumes greater than 150 mm-. These conditions in murine models may equate to the tumor burden in late-stage cancer patients. Our data indicates that our IL-12/PD-L2-Fc immunomodulatory molecules can successfully treat very large syngeneic tumors (equivalent to advanced, late-stage human cancer), suggesting promising applications in clinical settings.

### Example 5: In Vivo Efficacy of IL-12/PD-L2-Fc and IL-12/Anti-PD-1 Immunomodulatory Molecules in Established EMT6 Syngeneic Tumor Mice Model

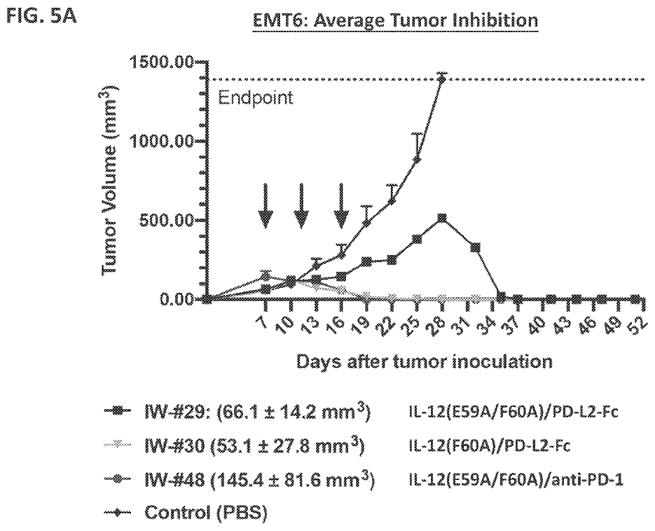

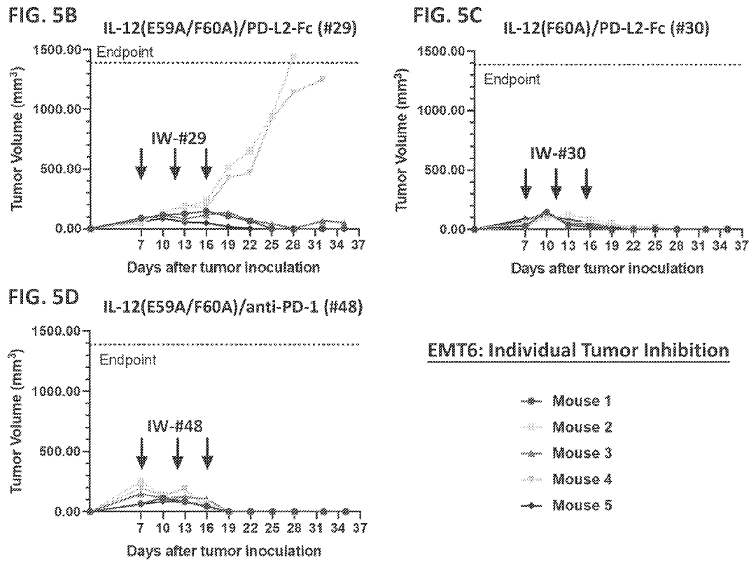

Mice (˜20g body weight) were inoculated with 0.25-:106 EMT6 murine breast cancer cells. Eleven days after tumor inoculation, tumor size was measured to be about 100-150 mm³. The average initial tumor volume plus or minus one standard deviation is given in parenthesis in the figure legend (FIG. 5A). After measuring tumor size, mice were injected with 200 μg (10 mg/kg) IL-12(E59A/F60A)/PD-L2-Fc, hinge (constructed in Example 2, IW-#29) immunomodulatory molecule (see FIG. 1G for structure (first polypeptide chain SEQ ID NO: 16, second polypeptide chain SEQ ID NO: 17)), 200 μg (10 mg/kg) IL-12(F60A)/PD-L2-Fc, hinge (constructed in Example 2, IW-#30) immunomodulatory molecule (see FIG. 1G for structure (first polypeptide chain SEQ ID NO: 16, second polypeptide chain SEQ ID NO: 18)), 200 μg (10 mg/kg) IL-12(E59A/F60A)/anti-PD-1, hinge (constructed in Example 1, IW-#48) immunomodulatory molecule (see FIG. 1C for structure (two light chains each comprising the amino acid sequence of SEQ ID NO: 50, one heavy chain comprising the amino acid sequence of SEQ ID NO: 21, and one heavy chain with the single-chain IL-12(E59A/F60A) variant positioned at the hinge region comprising the amino acid sequence of SEQ ID NO: 22), where in the Fab binds PD-1 but is not an agonist), or PBS (negative control). Each group had five mice. A total of three injections were given on days 7, 12, and 16 post-inoculation (indicated by black arrows). Tumor size was measured every 3 days since the first injection. Mice were sacrificed once tumor size reached over 1500 mm³. FIG. 5A depicts the average tumor volume in each treatment group. Individual mice plots for each group were also provided in FIG. 5B showing IL-12(E59A/F60A)/PD-L2-Fc, hinge (IW-#29) immunomodulatory molecule, FIG. 5C showing IL-12(F60A)/PD-L2-Fc, hinge (IW-430) immunomodulatory molecule, and FIG. 5D showing IL-12(E59A/F60A)/anti-PD-1, hinge (IW-#48) immunomodulatory molecule. IL-12(E59A/F60A)/PD-L2-Fc, hinge (IW-429) immunomodulatory molecule successfully inhibited tumor growth in 3/5 mice (60% cure rate) while IL-12(F60A)/PD-L2-Fc, hinge (IW-#30) immunomodulatory molecule and IL-12(E59A/F60A)/anti-PD-1, hinge (IW-#48) immunomodulatory molecule both successfully inhibited tumor growth in 5/5 mice (100% rate).

The initial average tumor size of the mouse group treated with IL-12(E59A/F60A)/anti-PD-1 immunomodulatory molecule (IW-#48) was more than twice of that of the other two test groups. These results showed that all three IL-12 immunomodulatory molecules tested could completely regress EMT6 syngeneic breast tumors in mice, with anti-PD-1 based immunomodulatory molecule having the best efficacy.

As seen in FIG. 5A, the tumor regression efficacy difference seen between IL-12(E59A/F60A)/PD-L2-Fc, hinge (IW-#29) immunomodulatory molecule and IL-12(F60A)/PD-L2-Fc, hinge (IW-#30) immunomodulatory molecule was likely due to lower potency (e.g., receptor binding and/or signal activation ability) of double mutation IL-12(E59A/F60A) compared to single mutation IL-12(F60A). Such efficacy difference may be compensated by higher dosing per injection (e.g., 20 mg/kg vs. 10 mg/kg), or more injections (e.g., increase from 3 to 5 injections) of IL-12(E59A/F60A)-based immunomodulatory molecules.

EMT6 mice model is moderately responsive to current immunotherapies. Combination treatment with anti-PD-1 and anti-CTLA-4 antibodies can significantly inhibit tumor growth but cannot completely regress the tumors. As can be seen from FIG. 5A, all IL-12/PD-L2-Fc and IL-12/anti-PD-1 immunomodulatory molecules tested were capable of regressing EMT6 tumors in 60-100% of mice, demonstrating promising in vivo efficacy.

### Example 6: IL-12/PD-L2-Fc and IL-12/Anti-PD-1 Immunomodulatory Molecules are Capable of Induce Specific, Anti-Tumor Memory in Cured EMT6 Syngeneic Tumor Mice Models

To investigate if immunomodulatory molecules described herein can function as cancer vaccine, or prevent cancer recurrence, a tumor re-challenge was conducted on all cured mice from Example 5. Thirty days after the final immunomodulatory molecule injection, cured mice were inoculated with 0.25×10⁶ EMT6 murine breast cancer cells on the right flank and 0.25×10⁶ CT26 murine colon cancer cells on the left flank (as control). Tumor sizes were recorded every 4 days following re-challenge tumor inoculation. Mice were sacrificed once tumor size reached over 1000 mm-.

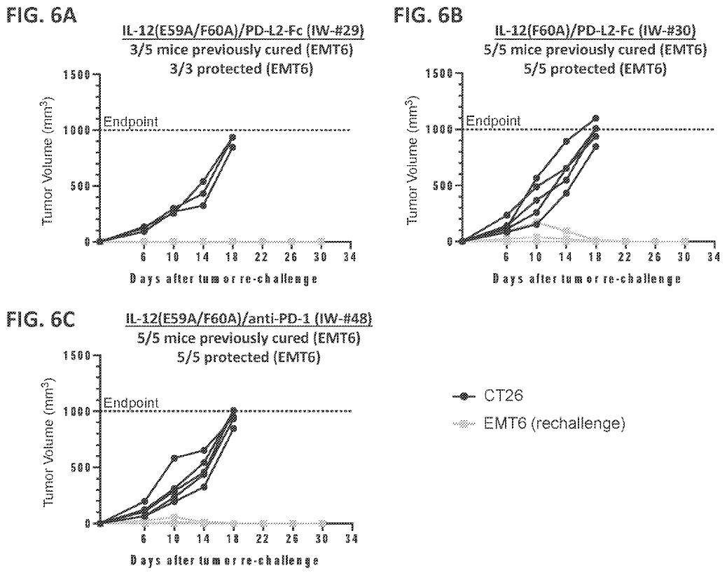

As seen in FIGS. 6A-6C, all cured mice previously treated with immunomodulatory molecules against EMT6 tumor were protected from EMT6 tumor re-challenge but not from CT26. Further, all three IL-12 immunomodulatory molecules demonstrated similar protection efficacy against EMT6 tumor re-challenge. These results indicate successful generation of anti-EMT6 tumor memory, suggesting that immunomodulatory molecules described herein, such as IL-12(E59A/F60A)/PD-L2 Fc immunomodulatory molecule (IW-#29), IL-12(F60A)/PD-L2-Fc immunomodulatory molecule (IW-#30), and IL-12(E59A/F60A)/anti-PD-1 immunomodulatory molecule (IW-#48), can serve as a cancer vaccine (e.g., against breast cancer (such as EMT6) tumors) in mice, and/or can prevent cancer recurrence, capable of inducing induce specific, anti-tumor memory.

### Example 7: In Vivo Efficacy of IL-12/PD-L2-Fc and IL-12/Anti-PD-1 Immunomodulatory Molecules Show that they can Significantly Inhibit Tumor Growth in Established 4T1 Triple Negative Breast Cancer (TNBC) Syngeneic Tumor Models

4T1 is a standard murine mammary tumor model used in preclinical studies on breast cancer metastasis. 4T1 is a refractory model for immunotherapy and does not respond to anti-PD-1, anti-CTLA-4, or combination of anti-PD-1 and anti-CTLA-4 antibody therapy.

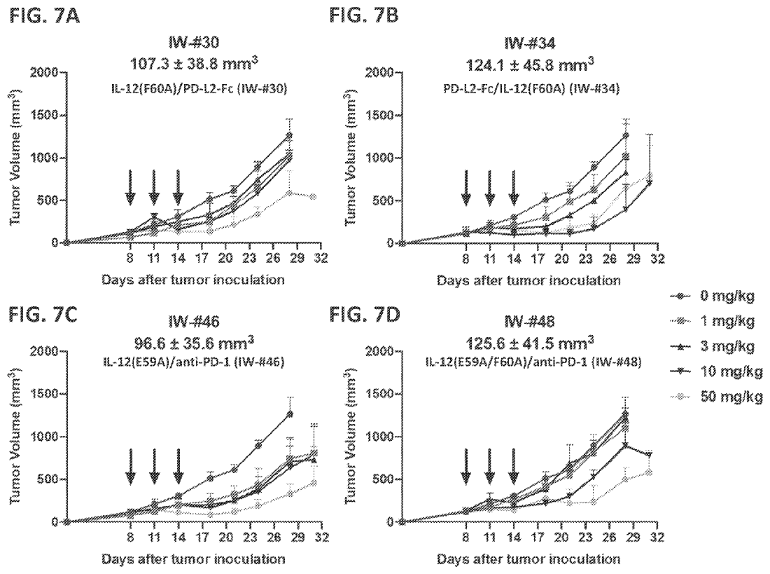

To test the therapeutic efficacy of immunomodulatory molecules described herein on immunotherapy-resistant cancer types, mice (˜20g body weight) were inoculated with 0.25×10⁶ 4T1 murine breast cancer cells. Seven days after tumor inoculation, tumor size was measured to be about 100 mm³. The average initial tumor volume plus or minus one standard deviation is given blow the figure title (FIG. 7A-7D). After measuring tumor size, mice were injected with increasing concentrations of IL-12(F60A)/PD-L2-Fc, hinge (constructed in Example 2, IW-#30) (first polypeptide chain SEQ ID NO: 16, second polypeptide chain SEQ ID NO: 18) immunomodulatory molecule (see FIG. 1G for structure), IL-12(E59A/F60A)/PD-L2-Fc, hinge (constructed in Example 2, TW-429) (first polypeptide chain SEQ ID NO: 16, second polypeptide chain SEQ ID NO: 17) immunomodulatory molecule (see FIG. 1G for structure), IL-12(F60A)/anti-PD-1, hinge (constructed in Example 1, TW-446) immunomodulatory molecule (see FIG. 1C for structure (two light chains each comprising the amino acid sequence of SEQ ID NO: 50, one heavy chain comprising the amino acid sequence of SEQ ID NO: 21, and one heavy chain with the single-chain IL-12(F60A) variant (SEQ ID NO: 71) positioned at the hinge region comprising the amino acid sequence of SEQ ID NO: 23), where in the Fab binds PD-1 but is not an agonist), and IL-12(E59A/F60A)/anti-PD-1, hinge (constructed in Example 1, IW-#48) (two light chains each comprising the amino acid sequence of SEQ ID NO: 50, one heavy chain comprising the amino acid sequence of SEQ ID NO: 21, and one heavy chain with the single-chain IL-12(E59A/F60A) variant (SEQ ID NO: 68) positioned at the hinge region comprising the amino acid sequence of SEQ ID NO: 22) immunomodulatory molecule (see FIG. 1C for structure, where in the Fab binds PD-1 but is not an agonist): 0, 1, 3, 10, and 50 mg/kg. Each group had five mice. The range was based on maximum tolerated doses of muIL12 (0.5 mg/kg) and reported IL-12 immunomodulatory molecules (<2.5 mg/kg). Our IL-12/PD-L2-Fc immunomodulatory molecules can reach doses of up to 50 mg/kg without seeing significant toxicity symptoms. A total of three injections were given on days 8, 11, and 14 post-inoculation (indicated by black arrows). Tumor size was measured every 3 days since the first injection. Mice were sacrificed once tumor size reached over 1500 mm³.

As can be seen from FIGS. 7A-7D, all IL-12(mut)/PD-L2-Fc, PD-L2-Fc/IL-12(mut), and IL-12(mut)/anti-PD-1 immunomodulatory molecules significantly inhibited 4T1 tumor growth, following a dose-dependent response. This demonstrating promising in vivo efficacy, especially considering that 4T1 is a refractory model that does not respond to anti-PD-1, anti-CTLA-4, or combination of anti-PD-1 and anti-CTLA-4 antibody therapy.

### Example 8: In Vivo Efficacy of IL-12/PD-L2-Fc Immunomodulatory Molecules Show that it can Significantly Inhibit Tumor Growth in Established B16-F10 Syngeneic Tumor Models

B16 a murine melanoma tumor cell line used for research as a model for human skin cancers. B16 is a refractory model for immunotherapy and does not respond to anti-PD-1, anti-CTLA-4, or combination of anti-PD-1 and anti-CTLA-4 antibody therapy.

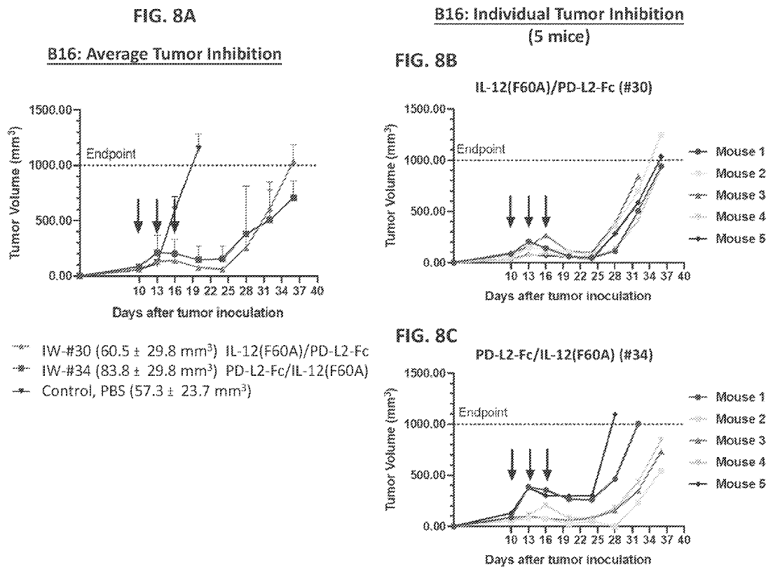

To test the therapeutic efficacy of immunomodulatory molecules described herein on more immunotherapy-resistant cancer types, mice (˜20g body weight) were inoculated with 0.25×10⁶ B16 murine melanoma cells. When tumor size reached about 50-100 mm³, mice were injected with 200 μg (10 mg/kg) IL-12(F60A)/PD-L2-Fc immunomodulatory molecule (constructed in Example 2; construct IW-#30; see FIG. 1G for structure), 200 μg (10 mg/kg) PD-L2-Fc/IL-12(F60A) immunomodulatory molecule (constructed in Example 2, construct IW-#34), or PBS (negative control). A total of three injections (10 mg/kg per injection) were given on days 10, 13, and 16 post-inoculation (indicated by black arrows in FIGS. 8A-8C). Tumor size was recorded over time. Mice were sacrificed once tumor size reached over 1000 mm³. Tumor size in parenthesis of FIG. 8A indicates average tumor size (±standard deviation) of each group when the first treatment was administered.

As seen in FIGS. 8A-8C, compared to PBS treatment group in which B16 tumor grew drastically since day 13 post-inoculation, PD-L2-Fc/IL-12(F60A) immunomodulatory molecule (IW-#34) and IL-12(F60A)/PD-L2-Fc immunomodulatory molecule (IW-#30) both significantly inhibited B16 tumor growth until after day 24 post-inoculation, indicating that both IL-12 immunomodulatory molecules can slow down tumor progression and/or extend life-span of individuals with immunotherapy-resistant cancers (e.g., melanoma), demonstrating promising in vivo efficacy.

### Example 9: In Vivo Efficacy of IL-12/PD-L2-Fc and PD-L2-Fc/IL-12 Immunomodulatory Molecules Show that it can Significantly Inhibit Tumor Growth in Established LL2 Syngeneic Tumor Models

LL2 murine lung carcinoma model is a refractory model for immunotherapy that does not respond to anti-PD-1, anti-CTLA-4, or combination of anti-PD-1 and anti-CTLA-4 antibody therapy.

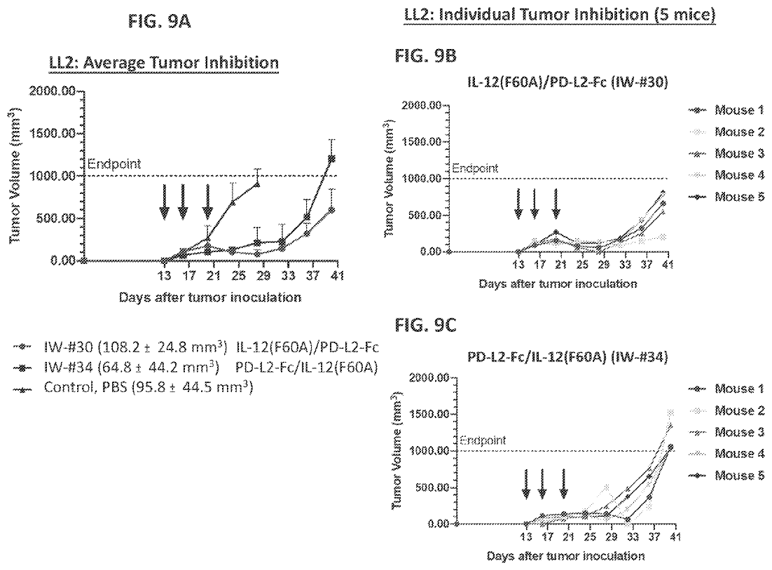

To test the therapeutic efficacy of immunomodulatory molecules described herein on more immunotherapy-resistant cancer types, mice (˜20g body weight) were inoculated with 0.25×10⁶ LL2 murine lung cancer cells. About 16 days after tumor inoculation, tumor size was measured to be about 50-100 mm³. The average initial tumor volume plus or minus one standard deviation is given in parenthesis (FIG. 9A). After measuring tumor size, mice were injected with 200 μg (10 mg/kg) IL-12(F60A)/PD-L2-Fc, hinge (constructed in Example 2, IW-#30) immunomodulatory molecule (see FIG. 1G for structure) or 200 μg (10 mg/kg) IL-12(F60A)/PD-L2-Fc, C-terminus of HC (constructed in Example 2, IW-#34) immunomodulatory molecule (see FIG. 1I for structure). PBS injection served as negative control. A total of three injections (10 mg/kg per injection) were given on days 13, 16, and 20 post-inoculation (indicated by black arrows). Tumor size was measured every 4 days since the first injection. Mice were sacrificed once tumor size reached over 1000 mm³.

As seen in FIGS. 9A-9C, compared to PBS treatment group in which LL2 tumor grew drastically since about day 20 post-inoculation, PD-L2-Fc/IL-12(F60A) immunomodulatory molecule (#IW-34) and IL-12(F60A)/PD-L2-Fc immunomodulatory molecule (IW-430) significantly inhibited LL2 tumor growth until after day 32-35 post-inoculation, indicating that both IL-12 immunomodulatory molecules can slow down tumor progression and/or extend life-span of individuals with immunotherapy-resistant cancers (e.g., lung cancer), demonstrating promising in vivo efficacy, especially considering that does not respond to anti-PD-1, anti-CTLA-4, or combination of anti-PD-1 and anti-CTLA-4 antibody therapy.

To summarize, data described herein (e.g., see Examples 7, 8, and 11) demonstrate promising in vivo efficacy of immunomodulatory molecules described herein (e.g., IL-12/PD-L2-Fc based immunomodulatory molecules) in treating various advanced and/or hard-to-treat cancer types (e.g., TNBC, melanoma, lung cancer), inhibiting cancer metastasis, treating or delaying tumor progression of cancer types that are resistant to current immunotherapies (e.g., anti-PD-1 therapy, anti-CTLA-4 therapy, or a combination therapy thereof), and/or extending life-span of such patients.

### Example 10: Replacing Anti-PD-1 Parental Antibody with PD-L2-Hinge-Fc Fusion Protein Significantly Reduces Toxicity of IL-12 Immunomodulatory Molecules

40 BALB/c mice were randomly divided into 16 groups (5 mice each group), and intraperitoneally injected with 200 μg or 1000 μg of: i) IL-12(F60A)/PD-L2-Fc immunomodulatory molecule (IW-#30), ii) IL-12(G64A)/PD-L2-Fc immunomodulatory molecule (constructed in Example 2), iii) IL-12(E59A/F60A)/PD-L2-Fc immunomodulatory molecule (IW-#29), iv) PD-L2-Fc/IL-12(F60A) immunocytokine (IL-12(F60A) moiety positioned at C′ of one Fc fragment; IW-#34), v) PD-L2-Fc/IL-12(E59A/F60A) immunocytokine (IL-12(E59A/F60A) moiety positioned at C′ of one Fc fragment), vi) IL-12(F60A)/anti-PD-1 immunomodulatory molecule (IW-#46), vii) IL-12(F60A)/anti-PD-1 immunomodulatory molecule (IW-#48), and viii) IL-12(G64A)/anti-PD-1 immunomodulatory molecule (“IW-#47”). These were constructed in Examples 1 and 2. Each group received intraperitoneal injections on Day I and Day 5. Mice were monitored daily for four parameters: i) fur texture, ii) reduced activity, iii) morbidity, and iv) weight loss greater than 10%.

As can be seen from Table 3, IL-12/anti-PD-1 immunomodulatory molecule comprising IL-12 (G64A) variant (IW-#47) showed the highest toxicity, as indicated by the death of 4/5 mice in low dose group and the death of 5/5 mice in high dose group. In contrast, treatment with IL-12/anti-PD-1 immunomodulatory molecule comprising IL-12 (F60A) variant (IW-#46) only induced one death in high dose group (1000 μg) and no death in low dose group (200 μg); treatment with IL-12/anti-PD-1 immunomodulatory molecule comprising IL-12 (E59A/F60A) variant (IW-#48) did not induce death in either dose. IL-12/anti-PD-1 immunomodulatory molecule comprising IL-12 double mutation E59A/F60A (IW-#48) also demonstrated less toxicity compared to that comprising IL-12 single F60A mutation (IW-#46), as indicated by the differences in severity of toxicity symptoms.

Among immunomodulatory molecules with IL-12 variant positioned at the hinge region, IL-12/PD-L2-Fc immunomodulatory molecule comprising IL-12 (G64A) variant showed the highest toxicity, as indicated by the death of 3/5 mice in low dose group and the death of 5/5 mice in high dose group. This is consistent with the highest toxicity results of IL-12 (G64A) among all IL-12 variants in IL-12/anti-PD-1 immunomodulatory molecule, and IL-12 (G64A) bioactivity shown in Example 1. When placing IL-12 (F60A) variant at the C-terminus of the PD-L2-hinge-Fc polypeptide (IW-#34), 2 out of 5 mice died in high dose group. In contrast, when IL-12 (F60A) variant was positioned at the hinge region of PD-L2-hinge-Fc polypeptide (IW-#30), all mice survived, even administered with high dose of immunomodulatory molecules (1000 μg).

IL-12/PD-L2-Fc immunomodulatory molecule comprising IL-12 double mutation E59A/F60A demonstrated less toxicity compared to that comprising IL-12 single F60A mutation, no matter IL-12(E59A/F60A) variant was positioned at the hinge region or at the C-terminus of Fc, as indicated by the differences in severity of toxicity symptoms. Dose-dependent toxicity was observed for most immunomodulatory molecules, as indicated by increased severity of toxicity symptoms such as worse fur texture, increased weight loss, and/or greater reduced activity when dose was increased from 200 μg to 1000 μg. IL-12(E59A/F60A)/PD-L2-Fc immunomodulatory molecule comprising IL-12 variant positioned at the hinge region (IW-#29) actually demonstrated the least toxicity in vivo among all IL-12/PD-L2-Fc, IL-12/anti-PD-1, and PD-L2-Fc/IL-12 immunomodulatory molecules, with 0 death rate and no toxicity symptom even when administered at high dose.

As can be seen from Table 3, our results indicate that replacing anti-PD-1 antigen-binding fragment with PD-L2 ligand in the IL-12-based immunomodulatory molecules can further reduce overall toxicity. For example, compare 0 death rate in high dose group of IL-12(F60A)/PD-L2-Fc (IW-#30) immunomodulatory molecule vs. 1/5 death rate in high dose group of IL-12(F60A)/anti-PD-1 (IW-#46) immunomodulatory molecule; compare 3/5 death rate in low dose group of IL-12(G64A)/PD-L2-Fc immunomodulatory molecule vs. 4/5 death rate in low dose group of IL-12(G64A)/anti-PD-1 immunomodulatory molecule (IW-#47). When comparing toxicity symptoms between the respective IL-12 variant immunomodulatory molecules, the lower toxicity of PD-L2-Fc based immunomodulatory molecules is even more obvious. For example, IL-12(E59A/F60A)/PD-L2-Fc (IW-#29) immunomodulatory molecule completely eliminated toxicity symptom compared to IL-12(E59A/F60A)/anti-PD-1 (IW-#48) immunomodulatory molecule, administered with either low or high dose; IL-12(F60A)/PD-L2-Fc (IW-#30) immunomodulatory molecule showed fewer toxicity symptoms (fur texture only) compared to those of IL-12(F60A)/anti-PD-1 (IW-#46) immunomodulatory molecule, either in low or high dose group. The reduced toxicity seen in PD-L2-Fc based IL-12 immunomodulatory molecules was likely due to stimulated PD-1 inhibitory immune checkpoint signaling upon PD-L2/PD-1 binding, which created an immunosuppression signal that “balances” against the immunostimulating/pro-inflammatory activity of IL-12. On the contrary, anti-PD-1 antibody (non-agonist Ab)-based IL-12 immunomodulatory molecules lack such immunosuppression signal, because they just bind to PD-1 but are not an agonist.

TABLE 3
In vivo toxicity of IL-12/PD-L2-Fc immunomodulatory molecules

| | | Second | | | | |
|---|---|---|---|---|---|---|
| | | binding | IL-12B | Dose | | |
| Name | Structure | domain | Mutation | (μg) | Deaths | Toxicity Symptoms |
| IL-12(F60A)/PD-L2-Fc | FIG. 1G | PD-L2 | F60A | 200 | None | Fur texture (moderate) |
| (IW-#30; hinge) | | Ligand | | 1000 | None | Far texture |
| IL-12(F60A)/anti-PD-1 | FIG. 1C | Anti-PD- | | 200 | None | Fur texture, reduced activity |
| (IW-#46; hinge) | | 1 Ab (not | | 1000 | 1/5 | Fur texture, reduced activity, |
| | | agonist) | | | | weight loss, morbidity |
| PD-L2-Fc/IL-12(F60A) | FIG. 1I | PD-L2 | | 200 | none | Fur texture, reduced activity, |
| (IW-#34; C-terminus) | | Ligand | | | | weight loss |
| | | | | 1000 | 2/5 | Fur texture, reduced activity, |
| | | | | | | weight loss, morbidity |
| IL-12(E59A/F60A)/PD- | FIG. 1G | PD-L2 | E59A/ | 200 | None | None |
| L2-Fc | | Ligand | F60A | 1000 | None | None |
| (IW-#29; hinge) | | | | | | |
| IL-12(E59A/F60A)/ | FIG. 1C | Anti-PD- | | 200 | None | Fur texture (moderate) |
| anti-PD-1 | | 1 Ab (not | | 1000 | None | Fur texture, reduced activity |
| (IW-#48; hinge) | | agonist) | | | | |
| PD-L2-Fc/IL- | FIG. 1I | PD-L2 | | 200 | None | Fur texture |
| 12(E59A/F60A) | | Ligand | | 1000 | None | Fur texture, reduced activity, |
| (C-terminus) | | | | | | weight loss |
| IL-12(G64A)/PD- | FIG. 1G | PD-L2 | G64A | 200 | 3/5 | Fur texture, reduced activity, |
| L2-Fc | | Ligand | | | | weight loss, morbidity |
| (hinge) | | | | 1000 | 5/5 | Fur texture, reduced activity, |
| | | | | | | weight loss, morbidity |
| IL-12(G64A)/anti-PD-1 | FIG. 1C | Anti-PD- | | 200 | 4/5 | Fur texture, reduced activity, |
| (IW-#47; hinge) | | 1 Ab (not | | | | weight loss, morbidity |
| | | agonist) | | 1000 | 5/5 | Fur texture, reduced activity, |
| | | | | | | weight loss, morbidity |

### Example 11: In Vivo Efficacy of IL-12 Based Immunomodulatory Molecules in 4T1 Triple Negative Breast Cancer (TNBC) Orthotopic Tumor Mice Model

4T1 is a standard murine mammary tumor model used in preclinical studies on breast cancer metastasis. 4T1 is a refractory model for immunotherapy and does not respond to anti-PD-1, anti-CTLA-4, or combination of anti-PD-1 and anti-CTLA-4 antibody therapy. Mammary fat pad injection of 4T1 can reproducibly generate 4T1 breast-cancer-derived lung metastases.

To test the therapeutic efficacy of immunomodulatory molecules described herein on immunotherapy-resistant cancer types as well as cancer metastasis, mice (˜20 g body weight) were inoculated with 0.25×10⁶ 4T1 murine breast cancer cells in the #3 mammary gland fat pad. Tumor development was monitored for approximately 21-30 days. Four days after tumor inoculation, mice were injected with 20 mg/kg (per injection) IL-12(E59A/F60A)/PD-L2-Fc immunomodulatory molecule (constructed in Example 2; construct IW-#29), 20 mg/kg (per injection) IL-12(F60A)/PD-L2-Fc immunomodulatory molecule (constructed in Example 2; construct IW-#30), a combination of 10 mg/kg anti-PD-1 antibody and 10 mg/kg anti-CTLA-4 antibody (per injection), or PBS (negative control). A total of five injections were given every four days. Mice were sacrificed after four weeks and primary tumor was extracted from the mammary fat pad.

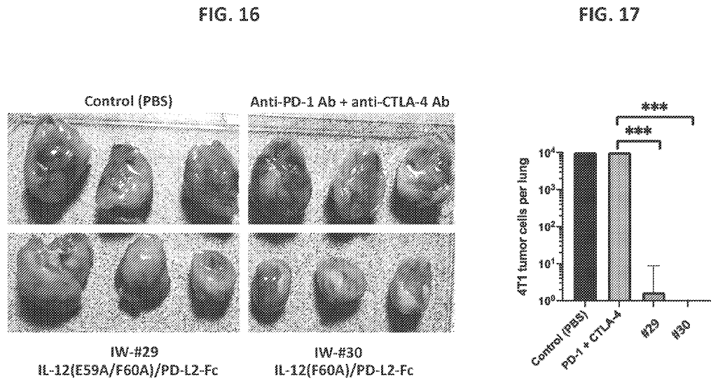

As seen in FIG. 16, compared to PBS control, IL-12(F60A)/PD-L2-Fc immunomodulatory molecule (IW-#30) inhibited 4T1 growth in mammary gland in all mice tested, IL-12(E59A/F60A)/PD-L2-Fc immunomodulatory molecule (IW-#29) inhibited 4T1 growth in mammary gland in 1 out of 3 mice tested, while anti-PD-1+anti-CTLA-4 combination treatment failed inhibiting 4T1 growth in mammary gland in all 3 mice tested. As discussed above, the efficacy difference was likely due to lower potency (e.g., receptor binding and/or signal activation ability) of double mutation IL-12(E59A/F60A) compared to single mutation IL-12(F60A). Such efficacy difference may be compensated by higher dosing per injection (e.g., 40 mg/kg vs. 20 mg/kg), or more injections (e.g., increase from 5 to 7 injections) of IL-12(E59A/F60A)-based immunomodulatory molecules.

To investigate the therapeutic efficacy on cancer metastasis, lungs were retrieved from sacrificed mice. Lung tissue was resuspended in collagenase/DNase solution and filtered through a 70 μm cell strainer. Cells were washed with PBS and resuspended in media. Four 1:10 serial dilutions were made. Cells were cultured in a 7% CO₂ incubator at 37° C. for 14 days to allow the formation of 4T1 cell colonies.

As seen in FIG. 17, IL-12(E59A/F60A)/PD-L2-Fc immunomodulatory molecule (IW-#29) and IL-12(F60A)/PD-L2-Fc immunomodulatory molecule (IW-#30) both significantly inhibited 4T1 metastasis in the lungs compared to the combination of anti-PD-1 and anti-CTLA-4 antibodies, or PBS (negative control). These findings were statistically significantly different (p-value <0.001).

These data demonstrating promising in vivo efficacy of IL-12 immunomodulatory molecules in treating advanced and/or hard-to-treat breast cancer (e.g., TNBC), inhibiting cancer metastasis, and possibly in treating other cancer types that are resistant to current immunotherapies.

### Example 12: Immunomodulatory Molecules with Cytokine Positioned at the Hinge Region Favor Target Antigen-Antibody (or Ligand-Receptor) Binding First, then Cytokine-Cytokine Receptor Binding Second

### Construction of IL-2 Variants and Immunomodulatory Molecules Thereof

IL-2/anti-PD-1 immunomodulatory molecule (“Fab-IL-2 mutant-Fc-PD-1 Ab”) was constructed similarly as in Example 1. An anti-human PD-1 antibody comprising nivolumab (Opdivo®) VH (SEQ ID NO: 48) and VL (SEQ ID NO: 49) sequences was used as the parental full-length antibody. The IL-2 variant comprising R38D/K43E/E61R triple mutations (SEQ ID NO: 26) was positioned within the hinge region of a heavy chain of the anti-PD-1 antibody (see FIG. 1C for exemplary structure, anti-PD-1 is antagonist Ab). The Fab-IL-2 mutant-Fc-PD-1 Ab immunomodulatory molecule (or “IL-2(R38D/K43E/E61R)/anti-PD-1 immunomodulatory molecule”) comprises two light chains each comprising the amino acid sequence of SEQ ID NO: 50, one heavy chain comprising the amino acid sequence of SEQ ID NO: 51, and one heavy chain with the IL-2 variant (SEQ ID NO: 26) positioned at the hinge region comprising the amino acid sequence of SEQ ID NO: 144.

The IL-2/PD-L2-Fc immunomodulatory molecule “ligand-IL-2 mutant-PD-L2-Fc” or “IL-2(R38D/K43E/E61R)/PD-L2-Fc immunomodulatory molecule” (“IW-#11” or “construct #11”) was constructed similarly as in Example 2. It comprises one fusion polypeptide (SEQ ID NO: 24) from N′ to C′: PD-L2 extracellular domain-GGGGS linker (SEQ ID NO: 213)-IL-2 variant (SEQ ID NO: 26)-N′ truncated IgG1 hinge (SEQ ID NO: 88)-an Fc fragment (SEQ ID NO: 97), and one pairing polypeptide (SEQ ID NO: 113) comprising from N′ to C′: PD-L2 extracellular domain-GGGGS linker (SEQ ID NO: 213)-N′ truncated IgG1 hinge (SEQ ID NO: 87)-a pairing Fc fragment (SEQ ID NO: 98).

Fab-IL-2 mutant-Fc-PD-1 Ab and ligand-IL-2 mutant-PD-L2-Fc were constructed, expressed, and purified as described in Example 1. FACS was used to confirm that both Fab-IL-2 mutant-Fc-PD-1 Ab and ligand-IL-2 mutant-PD-L2-Fc bind to HEK-PD-1-IL-2 cells (see below), but not to HEK-Blue™ IL-2 Cells (InvivoGen Cat #hkb-il2).

HEK-Blue™ IL-2 Cells and HEK-PD-1-IL-2 cells were used to assess IL-2 signal activation activity of Fab-IL-2 mutant-Fc-PD-1 Ab and ligand-IL-2 mutant-PD-L2-Fc, see below. Recombinant human IL-2 (rIL-2) served as positive control. Anti-PD-1 antibody nivolumab (Opdivo®) and parental PD-L2-Fc fusion protein (each polypeptide chain comprises SEQ ID NO: 111) served as negative controls. IL-2 signal transduction assay

HEK-Blue™ IL-2 Cells (InvivoGen Cat. #hkb-il2) and HEK-PD-1-IL-2 cells (generated in-house by overexpressing human PD-1 in HEK-Blue™ IL-2 Cells using a lentiviral vector) were used to assess IL-2 signal activation activity of the various IL-2 based immunomodulatory molecules, following the InvivoGen user manual (InvivoGen Cat. #hkb-il2), hereinafter also referred to as “HEK-IL-2 reporter assay” or “HEK-PD-1-IL-2 reporter assay.” HEK-Blue™ IL-2 reporter cells and HEK-PD-1-IL-2 reporter cells stably express the human TL-2 receptor (human IL-2Rα, TL-2Rβ, and IL-2Rγ), along with the human JAK3 and STAT5 genes to obtain a fully functional IL-2 signaling pathway. In addition, these reporter cells also carry a STAT5-inducible secreted embryonic alkaline phosphatase (SEAP) reporter gene. Upon IL-2 stimulation, HEK-Blue™ TL-2 reporter cells and HEK-PD-1-IL-2 reporter cells trigger the activation of STAT5 and the subsequent secretion of SEAP, the levels of which can be monitored using QUANTI-Blue™ (InvivoGen Cat. #rep-qbs) colorimetric enzyme assay for alkaline phosphatase activity.

Briefly, HEK-Blue™ IL-2 cells were added to various IL-2 based immunomodulatory molecules in each plate well (or recombinant human IL-2 in a well as positive control, anti-PD-1 antibody nivolumab (Opdivo®) in a well as negative control), and incubated at 37° C. in a CO₂ incubator for 20-24 hours or overnight. After incubation, supernatant was transferred to fresh plate wells, added QUANTI-Blue™ solution, and incubated at 37° C. incubator for 30 minutes-3 hours. Then SEAP levels were determined using a spectrophotometer at 620-655 nm. The activity of recombinant human IL-2 (positive control) in activating IL-2 signaling pathway was measured as 10 unit/ng and served as a reference. Percent IL-2 signal transduction for various IL-2 based immunomodulatory molecules was calculated by dividing the IL-2 based immunomodulatory molecule readout by the recombinant human IL-2 readout.

TABLE 4
IL-2 biological activity of Fab-IL-2 mutant-Fc-PD-1 Ab and ligand-IL-
2 mutant-PD-L2-Fc

| | Percent IL-2 signal | Percent IL-2 signal |
|---|---|---|
| | transduction (HEK-IL-2 | transduction (HEK-PD-1- |
| | reporter assay) | IL-2 reporter assay) |
| Recombinant human IL-2 (free state) | 100.0% | 100.0% |
| Anti-PD-1 antibody (nivolumab (Opdivo ®)) | 0.0% | 0.0% |
| Parental PD-L2-Fc fusion protein | 0.0% | 0.0% |
| Fab-IL-2 mutant-Fc-PD-1 Ab | 2.1% | 35.2% |
| (IL-2(R38D/K43E/E61R)/anti-PD-1) | | |
| ligand-IL-2 mutant-PD-L2-Fc | 2.4% | 43.5% |
| (IL-2(R38D/K43E/E61R)/PD-L2-Fc; IW-#11) | | |

Consistent with data shown above, in the absence of target antigen (PD-1) binding, IL-2 positioned at the hinge region of the immunomodulatory molecule showed little biological activity (2.1% or 2.4%) compared to free state rIL-2 (100.0%), as measured by HEK-IL-2 reporter assay. Comparing HEK-IL-2 reporter assay and HEK-PD-1-IL-2 reporter assay results in Table 4, binding of anti-PD-1 antigen-binding fragment or PD-L2 extracellular domain to PD-1 on cell surface greatly facilitated the engagement of L1-2 variant with IL-2 receptor. In other words, immunomodulatory molecules with cytokine positioned at the hinge region favored target antigen-antibody binding (Fab-LL-2 mutant-Fc-PD-1 Ab, 35.2% vs. 2.1%) or ligand-receptor binding (ligand-IL-2 mutant-PD-L2-Fc, 43.5% vs. 2.4%) first, then cytokine-cytokine receptor binding second.

IL-2/anti-HER2 and IL-2/anti-CD3 immunomodulatory molecules were constructed similarly, by positioning a mutant IL-2 moiety at one hinge region of an anti-HER2 full-length antibody comprising trastuzumab (Herceptin®) VH and VL, or an anti-CD3ε full-length antibody (made in-house), respectively. Anti-HER2/IL-2 and anti-CD3/IL-2 immunomodulatory molecules were constructed by positioning the same mutant IL-2 moiety at C-terminus of one heavy chain of the anti-HER2 full-length antibody or the anti-CD3$ full-length antibody. IL-2 mutant-Fc-Her2 Ab and IL-2 mutant-Fc-CD3 Ab were constructed by fusing the same IL-2 moiety to the N-terminus of one subunit of the Fc fragment of the anti-HER2 antibody or the anti-CD3ε antibody.

The immunomodulatory molecules were tested for activity using IL-2 signal transduction assay (HEK-Blue™ IL-2 cells, does not express CD3 or HER2) or PBMC proliferation assay. Results (data not shown) showed that cytokine (e.g., IL-2 variant) positioned at the hinge region of a heavy chain of a full-length antibody (e.g., anti-HER2 or anti-CD3 antibody) in the absence of binding of the antibody to the target antigen (e.g., HER2 or CD3) showed more restricted biological activity compared to when such cytokine was positioned at the N-terminus of a subunit of the Fc fragment, or at the C-terminus of a heavy chain of the full-length antibody. IL-2/anti-CD3 immunomodulatory molecule was able to bind to T cells via CD3, revealed the biological activity of cytokine positioned at the hinge region of one heavy chain of the anti-CD3 full-length antibody.

### PBMC Proliferation Assay

Biological activity of IL-2 can also be tested by peripheral blood mononuclear cell (PBMC) proliferation/survival assay. IL-2 is essential for the proliferation and survival of activated T-cells. Human PBMCs (80,000 cells/well) were stimulated by an anti-CD3 antibody (OKT3, 0.5 μg/mL) in the presence of increasing concentrations of recombinant human IL-2 (“rIL-2”; 0, 0.04, 0.2, 1.0, or 5.0 ng/mL). 1 ng/mL of rIL-2 was determined to be the minimal concentration required for T-cell proliferation, based on PBMC cell number (<80,000 cells/well) and viability after a 6-day culture. To determine the minimal concentration of IL-2 based immunomodulatory molecules required for T-cell proliferation, PBMCs (80,000 cells/well) were stimulated by an anti-CD3 antibody (OKT3, 0.5 μg/mL) in the presence of increasing concentrations of various formats of IL-2 based immunomodulatory molecules (0, 0.32, 1.6, 8, 40, 200, or 1000 ng/mL). Free state rIL-2 served as positive control (0.2 or 1 ng/mL). Percent T-cell proliferation of IL-2 based immunomodulatory molecule relative to rIL-2 was calculated by normalizing to corresponding molecular weights. For example, the molecular weights of IL-2/anti-HER2 immunomodulatory molecule and rIL-2 are about 162 kDa and 12 kDa, respectively, hence about 13 ng of IL-2/anti-HER2 immunomodulatory molecule is equivalent to about 1 ng of rIL-2 for the same IL-2 molar concentration.

### Example 13: Generation of IL-23/Anti-PD-1 Immunomodulatory Molecule (Fab-IL-23-Fc-PD-1 Ab) with IL-23 Biological Activity Directed to PD-1-Positive Cells

### Construction of IL-23 Variants and Immunomodulatory Molecules Thereof

IL-23 is a heterodimeric cytokine composed of p19 subunit and p40 subunit. The p40 subunit is shared with IL-12. IL-23 variants were constructed similarly as described in Example 1, by generating amino acid substitutions in the shared p40 subunit (see Table 5). A single chain IL-23 variant was made, from N′ to C′: p40 variant subunit (SEQ ID NOs: 63-66 and 140)-linker (SEQ ID NO: 229)-p19 wildtype subunit (SEQ ID NO: 73). A single chain “wildtype” IL-23 was also constructed as a control (SEQ ID NO: 74), from N′ to C′: p40 wildtype subunit-linker (SEQ ID NO: 229)-p19 wildtype subunit, referred to as “WT” in Table 5.

IL-23/anti-PD-1 immunomodulatory molecule (“Fab-IL-23(mut)-Fc-PD-1 Ab” or “Fab-IL-23(wt)-Fc-PD-1 Ab”) was constructed similarly as in Example 1. An anti-human PD-1 antibody comprising nivolumab (Opdivo®) VH (SEQ ID NO: 48) and VL (SEQ ID NO: 49) sequences was used as the parental full-length antibody. Various single chain IL-23 variants (or single chain “wildtype” IL-23 control) were positioned within the hinge region of a heavy chain of the anti-PD-1 antibody (see FIG. 1C for exemplary structure, anti-PD-1 is antagonist Ab). For example, Fab-IL-23(E59A/F60A)-Fc-PD-1 Ab immunomodulatory molecule comprising a single-chain IL-23 variant IL-12B (p40 E59A/F60A)-linker-IL-23A (wt p19) (SEQ ID NO: 75) positioned at the hinge region comprises two light chains each comprising the amino acid sequence of SEQ ID NO: 50, one heavy chain comprising the amino acid sequence of SEQ ID NO: 51, and one heavy chain with the single-chain IL-23 variant (SEQ ID NO: 75) positioned at the hinge region comprising the amino acid sequence of SEQ ID NO: 145. Immunomodulatory molecules were constructed, expressed, and purified as described in Example 1. The heavy chain comprising the amino acid sequence of SEQ ID NO: 51 can also be replaced with a heavy chain comprising the amino acid sequence of SEQ ID NO: 21.

### IL-23 Signal Transduction Assay

HEK-Blue™ IL-23 Cells (InvivoGen Cat. #hkb-il23) and HEK-PD-1-IL-23 cells (generated in-house by overexpressing human PD-1 in HEK-Blue™ IL-23 Cells using a lentiviral vector) were used to assess IL-23 signal activation activity of the various Fab-IL-23-Fc-PD-1 Ab immunomodulatory molecules comprising different IL-23 moieties, following the InvivoGen user manual (InvivoGen Cat. #hkb-il23), hereinafter also referred to as “HEK-IL-23 reporter assay” or “HEK-PD-1-IL-23 reporter assay.” HEK-Blue™ IL-23 reporter cells and HEK-PD-1-IL-23 reporter cells stably express the receptor complex consisting of IL-12Rβ1 and the IL-23 receptor (IL-23R), along with the human STAT3 gene to obtain a fully functional IL-23 signaling pathway (TyK2/JAK2/STAT3). In addition, these reporter cells also carry a STAT3-inducible SEAP reporter gene. Upon IL-2 stimulation, HEK-Blue™ 11-23 reporter cells and HEK-PD-1-IL-23 reporter cells trigger the activation of STAT3 and the subsequent secretion of SEAP, the levels of which can be monitored using QUANTI-Blue™ (InvivoGen Cat. #rep-qbs) colorimetric enzyme assay for alkaline phosphatase activity. Experimental procedure was similar as described in Example 12 for IL-2 signal transduction assay. Recombinant human IL-23 (rIL-23) in free state served as positive control and reference for percent activity calculation.

TABLE 5
IL-23 biological activity of Fab-IL-23-Fc-PD-1 Abs comprising different IL-23 moieties

| | rIL-23 | “WT” | G64A | E59A | F60A | E59A/F60A |
|---|---|---|---|---|---|---|
| HEK-IL-23 cells | 100.0% | 70.0% | 56.0% | 6.9% | 8.2% | 0.0% |
| HEK-PD-1-IL-23 cells | 100.0% | 150.0% | 180.0% | 39.0% | 46.0% | 4.8% |

As can be seen from Table 5, positioning IL-23 comprising a wildtype p40 subunit at the hinge region retained IL-23 activity of about 70.0%, even in the absence of target antigen (PD-1)-antibody binding in HEK-IL-23 reporter cells. E59A and F60A mutations in p40 subunit significantly reduced IL-23 activity to about 6.9% or 8.2% in the absence of PD-1/anti-PD-1 antibody binding, which was rescued to about 39.0% or 46.0% in the presence of PD-1/anti-PD-1 antibody binding in HEK-PD-1-IL-23 cells. Fab-IL-23-Fc-PD-1 Ab comprising E59A/F60A double mutations in IL-23 p40 subunit (“Fab-IL-23(E59A/F60A)-Fc-PD-1 Ab”) demonstrated PD-1-positive cell specific IL-23 biological activity (4.8%), with no cross reactivity with PD-1-negative cells (0.0%). These data demonstrate successful generation of anti-PD-1 antibody-based immunomodulatory molecules that can specifically target cytokine (e.g., IL-23) biological activity towards PD-1+ cells.

IL-23/anti-CD4 immunomodulatory molecules were similarly generated using an anti-CD4 antibody comprising Ibalizumab (Trogarzo®) V_{H} and V_{L} as parental Ab, and placing IL-23 moiety at one hinge region of the full-length anti-CD4 antibody. The IL-23 biological activity of various Fab-IL-23-Fc-CD4 Abs was measured using the IFN-γ release assay. rIL-23 served as positive control and percent activity reference. Positioning IL-23 comprising a wildtype p40 subunit at the hinge region still retained IL-23 activity of about 21.0%, even in the absence of target antigen (CD4)-antibody binding in CD8+ T cells; the activity was 34.0% with the presence of CD4/anti-CD4 antibody binding. E59A and F60A mutations in p40 subunit significantly reduced IL-23 activity to about 2.0/6 or 1.5% in the absence of CD4/anti-CD4 antibody binding, which was rescued to about 24.0% or 29.0% in the presence of CD4+ T cells/anti-CD4 antibody binding. Fab-IL-23-Fc-CD4 Ab comprising E59A/F60A double mutations in IL-23 p40 subunit (“Fab-IL-23(E59A/F60A)-Fc-CD4 Ab”) demonstrated CD4+ T cells specific IL-23 biological activity (6.8%), with no cross reactivity with CD8+ T cells (0.0%). These demonstrate successful generation of anti-CD4 antibody-based immunomodulatory molecules that can specifically target cytokine (e.g., IL-23) biological activity towards CD4-positive cells.

### Interferon-Gamma Release Assay (IGRA) for Measuring IL-12 or IL-23 Biological Activity

IL-23 and IL-12 can stimulate activated CD4+ or CD8+ T cells to release IFN-γ. The biological activity of IL-12 or IL-23 can be measured by the amount of IFN-γ released from activated T cells. Binding of IL-12 to its receptor (heterodimeric receptor composed of IL-12R-01 and IL-12R-β2 subunits) triggers a signaling pathway involving TyK2 (tyrosine kinase 2), JAK2 (Janus kinase 2) and STAT4 (signal transducer and activator of transcription 4) which results in the production of IFN-γ. CD4+ T cells or CD8+ T cells were isolated from PBMC, and stimulated by anti-CD3 antibody (1 μg/mL OKT3) in the presence of recombinant human IL-2 (30 units/mL) for 5 days. After 5 days, the activated CD4+ or CD8+ T cells (80,000 cells/well) were cultured in the presence of increasing concentrations of recombinant human IL-12 (rIL-12) or recombinant human IL-23 (rIL-23) (0, 0.62, 1.25, 2.5, 5, 10, or 20 ng/mL). The following day, the amount of IFN-γ released into the cell culture medium was measured by an ELISA assay. Percent IL-12 or IL-23 biological activity was calculated by dividing the readout of IL-12-based immunomodulatory molecules or IL-23-based immunomodulatory molecules by the readout of rIL-12 or rIL-23.

The minimal concentration of rIL-12 required to stimulate the release of IFN-γ from activated T cells was 2.5 ng/ml, while for rIL-23 it was 5 ng/ml. To determine the minimal concentration of the IL-12-based immunomodulatory molecules or IL-23-based immunomodulatory molecules to observe a positive biological response, activated CD4+ or CD8+ T cells (80,000 cells/well) were cultured overnight in the presence of increasing concentrations of IL-12-based immunomodulatory molecules or IL-23-based immunomodulatory molecules (0, 0.32, 1.6, 8, 40, 200, or 1000 ng/mL). rIL-12 (2.5 ng/mL) or rIL-23 (5 ng/mL) served as positive control. The percent biological activity of the IL-12-based immunomodulatory molecule or IL-23-based immunomodulatory molecule relative to corresponding free state cytokine (rIL-12 or rIL-23) was calculated by normalizing to corresponding molecular weights. The molecular weights of IL-12-based immunomodulatory molecule and rIL-12 are about 220 kDa and about 70 kDa, respectively. The molecular weights of IL-23-based immunomodulatory molecules and rIL-23 are about 215 kDa and about 65 kDa, respectively. Hence, about 3 ng of IL-12-based immunomodulatory molecule or TL-23-based immunomodulatory molecule is equivalent to about 1 ng of rIL-12 or rIL-23 for the same IL-12 or IL-23 molar concentration.

### Example 14: Generation of IL-10/Anti-PD-1 Immunomodulatory Molecule (Fab-IL-10-Fe-PD-1 Ab) with IL-10 Biological Activity Directed to PD-1-Positive Cells

### Construction of IL-10 Variants and Immunomodulatory Molecules Thereof

IL-10 is naturally expressed as a non-covalently linked homodimer. IL-10 variants were constructed by replacing amino acids from position 24 to 32 with Alanine or Serine (see Table 6), and a single chain IL-10 variant was made, from N′ to C′: IL-10 variant subunit (SEQ ID NOs: 53-58)-linker (SEQ ID NO: 227)-IL-10 variant subunit (SEQ ID NOs: 53-58). A single chain “wildtype” IL-10 was also constructed as a control (SEQ ID NO: 59), from N′ to C′: IL-10 wildtype subunit (SEQ ID NO: 52)-linker (SEQ ID NO: 227)-IL-10 wildtype subunit, referred to as “WT” in Table 6.

IL-10/anti-PD-1 immunomodulatory molecule (“Fab-IL-10(mut)-Fc-PD-1 Ab” or “Fab-IL-10(wt)-Fc-PD-1 Ab”) was constructed similarly as in Example 1. An anti-human PD-1 antibody comprising nivolumab (Opdivo®) VH (SEQ ID NO: 48) and VL (SEQ ID NO: 49) sequences was used as the parental full-length antibody. Various single chain IL-10 variants (or single chain “wildtype” IL-10 control) were positioned within the hinge region of a heavy chain of the anti-PD-1 antibody (see FIG. 1C for exemplary structure, anti-PD-1 is antagonist Ab). For example, Fab-IL-10(R27A)-Fc-PD-1 Ab immunomodulatory molecule (“IL-10(R27A)/anti-PD-1”) comprising a single-chain IL-10 variant IL-10(R27A)-linker-IL-10(R27A) (SEQ ID NO: 60) positioned at the hinge region comprises two light chains each comprising the amino acid sequence of SEQ ID NO: 50, one heavy chain comprising the amino acid sequence of SEQ ID NO: 51, and one heavy chain with the single-chain IL-10 variant (SEQ ID NO: 60) positioned at the hinge region comprising the amino acid sequence of SEQ ID NO: 146. The heavy chain comprising the amino acid sequence of SEQ ID NO: 51 can also be replaced with a heavy chain comprising the amino acid sequence of SEQ ID NO: 21. Immunomodulatory molecules were constructed, expressed, and purified as described in Example 1. IL-10 signal transduction assay

HEK-Blue™ IL-10 Cells (InvivoGen Cat. #hkb-il10) and HEK-PD-1-IL-10 cells (generated in-house by overexpressing human PD-1 in HEK-Blue™ IL-10 Cells using a lentiviral vector) were used to assess IL-10 signal activation activity of the various Fab-IL-10-Fc-PD-1 Abs comprising different IL-10 moieties, following the InvivoGen user manual (InvivoGen Cat. #hkb-il10), hereinafter also referred to as “HEK-IL-10 reporter assay” or “HEK-PD-1-IL-10 reporter assay.” HEK-Blue™ IL-10 reporter cells and HEK-PD-1-IL-10 reporter cells stably express IL-10 receptor hIL-10Rα. and hIL-10Rβ chains, human STAT3, and STAT3-inducible SEAP. Binding of IL-10 to its receptor on the surface of HEK-Blue™ IL-10 cells or HEK-PD-1-IL-23 reporter cells triggers JAK1/STAT3 signaling and the subsequent production of SEAP, the level of which in the cell culture supernatant can be monitored using QUANTI-Blue™ (InvivoGen Cat. #rep-qbs). Experimental procedure was similar as described in Example 12 for IL-2 signal transduction assay. Recombinant human IL-10 (rIL-10) in free state served as positive control and reference for percent activity calculation.

TABLE 6
IL-10 biological activity of Fab-IL-10-Fc-PD-1 Abs comprising different IL-10 moieties

| | rIL-10 | “WT” | R24A | D25A/L26A |
|---|---|---|---|---|
| HEK-IL-10 cells | 100.0% | 26.0% | 15.0% | 4.0% |
| HEK-PD-1-IL-10 cells | 100.0% | 200.0% | 150.0% | 56.0% |
| | R27A | D28A/A29S | F30A/S31A | R32A |
| HEK-IL-10 cells | <0.1% | 17.0% | 13.0% | 10.0% |
| HEK-PD-1-IL-10 cells | 21.0% | 70.0% | 67.0% | 42.0% |

As can be seen from Table 6, positioning IL-10 comprising wildtype IL-10 subunit at the hinge region still retained IL-10 activity of about 26.0%, even in the absence of target antigen (PD-1)-antibody binding in HEK-IL-10 cells. All IL-10 variants tested reduced IL-10 activity in the absence of PD-1/anti-PD-1 antibody binding compared to that of “wildtype” IL-10, and their IL-10 activity was rescued in the presence of PD-1/anti-PD-1 antibody binding in HEK-PD-1-IL-10 cells. Fab-IL-10-Fc-PD-1 Ab comprising R27A mutation in IL-10 (“Fab-IL-10(R27A)-Fc-PD-1 Ab”) demonstrated PD-1-positive cell specific IL-10 biological activity (21.0%), with minimal cross reactivity with PD-1-negative cells (<0.1%). These data demonstrate successful generation of anti-PD-1 antibody-based immunomodulatory molecules that can specifically target cytokine (e.g., IL-10) biological activity towards PD-1-positive cells.

### Example 15: Generation of IFN-γ/Anti-PD-1 Immunomodulatory Molecule (Fab-IFN-γ-Fc-PD-1 Ab) with IFN-γ Biological Activity Directed to PD-1-Positive Cells

### Construction of IFN-γ Variants and Immunomodulatory Molecules Thereof

IFN-γ is naturally expressed as a symmetric homodimer. IFN-γ variants were constructed by replacing amino acids from position 20 to 25 with A, K, S, E, Q, or V (see Table 7), and a single chain IFN-γ variant was made, from N′ to C′: IFN-γ variant subunit (SEQ ID NOs: 39-45)-linker (SEQ ID NO: 227)-IFN-γ variant subunit (SEQ ID NOs: 39-45). A single chain “wildtype” IFN-γ was also constructed as a control (SEQ ID NO: 46), from N′ to C′: IFN-γ wildtype subunit (SEQ ID NO: 38)-linker (SEQ ID NO: 227)-IFN-γ wildtype subunit (SEQ ID NO: 38), referred to as “WT” in Table 7.

IFN-γ/anti-PD-1 immunomodulatory molecule (“Fab-IFN-γ(mut)-Fc-PD-1 Ab” or “Fab-IFN-γ(wt)-Fc-PD-1 Ab”) was constructed similarly as in Example 1. An anti-human PD-1 antibody comprising nivolumab (Opdivo®) VH (SEQ ID NO: 48) and VL (SEQ ID NO: 49) sequences was used as the parental full-length antibody. Various single chain IFN-γ variants (or single chain “wildtype” IFN-γ control) were positioned within the hinge region of a heavy chain of the anti-PD-1 antibody (see FIG. 1C for exemplary structure, anti-PD-1 is antagonist Ab). For example, Fab-IFN-γ(A23V)-Fc-PD-1 Ab immunomodulatory molecule (“IFN-γ(A23V)/anti-PD-1”) comprising a single-chain IFN-γ variant IFN-γ(A23V)-linker-IFN-γ(A23V) (SEQ ID NO: 47) positioned at the hinge region comprises two light chains each comprising the amino acid sequence of SEQ ID NO: 50, one heavy chain comprising the amino acid sequence of SEQ ID NO: 51, and one heavy chain with the single-chain IFN-γ variant (SEQ ID NO: 47) positioned at the hinge region comprising the amino acid sequence of SEQ ID NO: 147. The heavy chain comprising the amino acid sequence of SEQ ID NO: 51 can also be replaced with a heavy chain comprising the amino acid sequence of SEQ ID NO: 21. The single-chain homodimer IFN-γ (A23V/A23V) variant can comprise sequence of SEQ ID NO: 47 or 252. Immunomodulatory molecules were constructed, expressed, and purified as described in Example 1.

### IFN-γ Signal Transduction Assay

HEK-Blue™ IFN-γ Cells (InvivoGen Cat. #hkb-ifng) and HEK-PD-1-IFN-γ cells (generated in-house by overexpressing human PD-1 in HEK-Blue™ IFN-γ Cells using a lentiviral vector) were used to assess IFN-γ signal activation activity of the various Fab-IFN-γ-Fc-PD-1 Abs comprising different IFN-γ moieties, following the InvivoGen user manual (InvivoGen Cat. #hkb-ifng), hereinafter also referred to as “HEK-IFN-γ reporter assay” or “HEK-PD-1-IFN-γ reporter assay.” HEK-Blue™ IFN-γ reporter cells and HEK-PD-1-IFN-γ reporter cells stably express human STAT1 gene, and STAT1-inducible SEAP. The other genes of the pathway are naturally expressed in sufficient amounts in the reporter cells. Binding of IFN-γ to its heterodimeric receptor consisting of IFNGR1 and IFNGR2 chains on the surface of HEK-Blue™ IFN-γ cells or HEK-PD-1-IFN-γ reporter cells triggers JAK1/JAK2/STAT1 signaling and the subsequent production of SEAP, the level of which in the cell culture supernatant can be monitored using QUANTI-Blue™ (InvivoGen Cat #rep-qbs). Experimental procedure was similar as described in Example 12 for IL-2 signal transduction assay. Recombinant human IFN-γ (rIFN-γ) in free state served as positive control and reference for percent activity calculation.

TABLE 7
IFN-γ biological activity of Fab-IFN-γ-Fc-PD-1 Abs comprising different IFN-γ moieties

| | rIFN-γ | “WT” | S20A/D21A | D21K | V22A/A23S |
|---|---|---|---|---|---|
| HEK-IFN-γ cells | 100.0% | 36.0% | 30.0% | 35.0% | 1.2% |
| HEK-PD-1-IFN-γ cells | 100.0% | 130.0% | 89.0% | 110.0% | 24.0% |

| | D24A/N25A | A23E/D24E/N25K | A23Q | A23V |
|---|---|---|---|---|
| HEK-IFN-γ cells | 21.0% | 0.2% | 0.8% | 0.6% |
| HEK-PD-1-IFN-γ cells | 70.0% | 23.0% | 31.0% | 27.0% |

As can be seen from Table 7, positioning IFN-γ comprising wildtype IFN-γ subunit at the hinge region retained IFN-γ activity of about 36.0%, even in the absence of target antigen (PD-1)-antibody binding in HEK-IFN-γ cells. A23 residue appears critical for IFN-γ biological activity, as all IFN-γ variants comprising A23 mutation greatly reduced IFN-γ activity in the absence of PD-1/anti-PD-1 antibody binding compared to that of “wildtype” IFN-γ, and their IFN-γ activity was rescued in the presence of PD-1/anti-PD-1 antibody binding in HEK-PD-1-IFN-γ cells. Fab-IFN-γ-Fc-PD-1 Ab comprising A23V mutation in IFN-γ (“Fab-IFN-γ(A23V)-Fc-PD-1 Ab”) demonstrated PD-1-positive cell specific IFN-γ biological activity (27.0%), with minimal cross reactivity with PD-1-negative cells (0.6%). Fab-IFN-γ-Fc-PD-1 Ab comprising A23E/D24E/N25K triple mutations in IFN-γ (“Fab-IFN-γ(A23E/D24E/N25K)-Fc-PD-1 Ab”) demonstrated PD-1-positive cell specific IFN-γ biological activity (23.0%), with minimal cross reactivity with PD-1-negative cells (0.2%). These data demonstrate successful generation of anti-PD-1 antibody-based immunomodulatory molecules that can specifically target cytokine (e.g., IFN-γ) biological activity towards PD-1-positive cells.

IFN-γ/anti-CD4 immunomodulatory molecules were similarly generated, and showed similar IFN-γ activities as IFN-r/anti-PD-1 immunomodulatory molecules (data now shown). IFN-γ can induce PD-L1 expression on cell surface. All IFN-γ variants comprising A23 mutation greatly reduced IFN-γ activity close to baseline level in the absence of CD4/anti-CD4 antibody binding compared to that of “WT” IFN-γ, and their IFN-γ activity was rescued in the presence of CD4/anti-CD4 antibody binding. Fab-IFN-γ-Fc-CD4 Ab comprising A23E/D24E/N25K triple mutations in IFN-γ (“Fab-IFN-γ(A23E/D24E/N25K)-Fc-CD4 Ab”) or A23V mutation (“Fab-IFN-γ(A23V)-Fc-CD4 Ab”) demonstrated CD4+ cell specific IFN-γ biological activity, with no or little cross reactivity with CD4-negative cells. These demonstrate successful generation of anti-CD4 antibody-based immunomodulatory molecules that can specifically target cytokine (e.g., IFN-γ) biological activity towards CD4-positive cells.

### Example 16: Generation of IFN-α2b/Anti-PD-1 Immunomodulatory Molecule (Fab-IFN-α2b-Fc-PD-1 Ab) with IFN-α2b Biological Activity Directed to PD-1-Positive Cells

### Construction of IFN-α2b Variants and Immunomodulatory Molecules Thereof

IFN-α2b (Intron-A®) is an antiviral or antineoplastic drug. It is a recombinant form of IFN-α2. IFN-α2b variants were constructed by replacing amino acids at positions 30 and 32-34 with Alanine (SEQ ID NOs: 32, 34, 35, and 36; see Table 8).

IFN-α2b/anti-PD-1 immunomodulatory molecule (“Fab-IFN-α2b(mut)-Fc-PD-1 Ab” or “Fab-IFN-α2b(wt)-Fc-PD-1 Ab”) was constructed similarly as in Example 1. An anti-human PD-1 antibody comprising nivolumab (Opdivo®) V_{H} (SEQ ID NO: 48) and V_{L} (SEQ ID NO. 49) sequences was used as the parental full-length antibody. Various IFN-α2b variants (or wildtype IFN-α2b control “WT”) were positioned within the hinge region of a heavy chain of the anti-PD-1 antibody (see FIG. 1C for exemplary structure, anti-PD-1 is antagonist Ab). For example, the Fab-IFN-α2b(L30A)-Fc-PD-1 Ab immunomodulatory molecule (“IFN-α2b(L30A)/anti-PD-1”) comprises two light chains each comprising the amino acid sequence of SEQ ID NO: 50, one heavy chain comprising the amino acid sequence of SEQ ID NO: 51, and one heavy chain with the IFN-α2b(L30A) variant (SEQ ID NO: 32) positioned at the hinge region comprising the amino acid sequence of SEQ ID NO: 148. The heavy chain comprising the amino acid sequence of SEQ ID NO: 51 can also be replaced with a heavy chain comprising the amino acid sequence of SEQ ID NO: 21 or a heavy chain comprising a different linker (e.g., GSGGGGG; SEQ ID NO: 206) at the hinge region. Immunomodulatory molecule were constructed, expressed, and purified as described in Example 1.

### IFN-α/β Signal Transduction Assay

HEK-Blue™ IFN-α/D cells (InvivoGen Cat. #hkb-ifnab) and HEK-PD-1-IFN-α/β cells (generated in-house by overexpressing human PD-1 in HEK-Blue™ IFN-α/0 cells using a lentiviral vector) were used to assess IFN-α2b signal activation activity of the various Fab-IFN-α2b-Fc-PD-1 Abs comprising different IFN-α2b moieties, following the InvivoGen user manual (InvivoGen Cat. #hkb-ifnab), hereinafter also referred to as “HEK-IFN-α/β reporter assay” or “HEK-PD-1-IFN-α/β reporter assay.” HEK-Blue™ IFN-α/β reporter cells and HEK-PD-1-IFN-α/β reporter cells were generated by stable transfection of HEK293 cells with the human STAT2 and IRF9 genes to obtain a fully active type I IFN signaling pathway, and inducible SEAP under the control of IFN-α/s inducible ISG54 promoter. The other genes of the pathway (IFNAR1, IFNAR2, JAK1, TyK2, and STAT1) are naturally expressed by these cells. Binding of IFN-α or IFN-β to its heterodimeric receptor consisting of IFNAR1 and IFNAR2 chains triggers JAK/STAT/ISGF3 signaling and subsequent production of SEAP, the level of which in the cell culture supernatant can be monitored using QUANTI-Blue™ (InvivoGen Cat. #rep-qbs). Experimental procedure was similar as described in Example 12 for IL-2 signal transduction assay. IFN-α2b in free state served as positive control and reference for percent activity calculation.

TABLE 8
IFN-α2b biological activity of Fab-IFN-α2b-Fc-PD-1
Abs comprising different IFN-α2b moieties

| | IFN-α2b | | | | | |
|---|---|---|---|---|---|---|
| | (free state) | “WT” | L30A | D32A | R33A | H34A |
| HEK-IFN-α/β cells | 100.0% | 54.0% | 15.0% | 9.0% | 5.0% | 20.0% |
| HEK-PD-1-IFN-α/β cells | 100.0% | 96.0% | 110.0% | 50.0% | 25.0% | 56.0% |

As can be seen from Table 8, positioning wildtype IFN-α2b at the hinge region of the anti-PD-1 antibody retained IFN-α2b activity of about 54.0%, even in the absence of target antigen (PD-1)-antibody binding in HEK-IFN-α/0 cells. L30, D32, R33, and H34 residues all appear critical for IFN-α2b biological activity, as all IFN-α2b variants greatly reduced IFN-α2b activity in the absence of PD-1/anti-PD-1 antibody binding compared to that of wildtype IFN-α2b, and their IFN-α2b activity was rescued in the presence of PD-1/anti-PD-1 antibody binding in HEK-PD-1-IFN-α/β cells. Fab-IFN-α2b-Fc-PD-1 Ab comprising L30A mutation in IFN-α2b (“Fab-IFN-α2b(L30A)-Fc-PD-1 Ab”) demonstrated PD-1-positive cell specific IFN-α2b biological activity (110.0%), with greatly reduced cross reactivity with PD-1-negative cells (15.0%). Fab-IFN-α2b-Fc-PD-1 Ab comprising R33A mutation in IFN-α2b (“Fab-IFN-α2b(R33A)-Fc-PD-1 Ab”) demonstrated PD-1-positive cell specific IFN-α2b biological activity (25.00%6), with greatly reduced cross reactivity with PD-1-negative cells (5.0%). These data demonstrate successful generation of anti-PD-1 antibody-based immunomodulatory molecules that can specifically target cytokine (e.g., IFN-α2b) biological activity towards PD-1-positive cells, with reduced cytokine biological activity towards PD-1-negative cells.

### Example 17: Placing IL-2 Variant at the Hinge Region of the IL-2/PD-L2-Fc Immunomodulatory Molecules Significantly Reduces Toxicity in Mice

IL-2/PD-L2-Fc (hinge) and PD-L2-Fc/IL-2 (C-terminal) immunomodulatory molecules were similarly constructed as in Examples 2 and 12.

25 BALB/c mice were randomly divided into 5 groups (5 mice each group), and intraperitoneally injected with 200 μg or 1000 μg of IL-2(R38D/K43E/E61R)/PD-L2-Fc immunomodulatory molecule (“IW-#11” or “construct #11”; constructed in Example 12), PD-L2-Fc/IL-2(R38D/K43E/E61R) immunomodulatory molecule (IL-2(R38D/K43E/E61R) moiety (SEQ ID NO: 26) fused to the C′ of one Fc fragment of a parental PD-L2-hinge-Fc fusion protein), or parental PD-L2-hinge-Fc fusion protein (two PD-L2-hinge-Fc polypeptides each comprising SEQ ID NO: 111) as control. Each group received intraperitoneal injections on Day I and Day 5. Mice were monitored daily for four parameters: i) fur texture, ii) reduced activity, iii) morbidity, and iv) weight loss greater than 10%.

As can be seen from Table 9, when placing IL-2 variant at the C-terminus of the PD-L2-hinge-Fc polypeptide, five out of five mice died 4-6 days post injection, in both high and low dose groups. In contrast, when IL-2 variant was positioned at the hinge region, all mice survived, even administered with high dose of immunomodulatory molecules (1000 μg). Same survival rate was observed in the control group (PD-L2-Fc without IL-2 fusion). For immunomodulatory molecules with IL-2 variant positioned at the hinge region, toxicity appeared to be dose-dependent, as indicated by increased weight loss and greater reduced activity when dose was increased from 200 μg to 1000 μg.

TABLE 9
In vivo toxicity of IL-2/PD-L2-Fc immunocytokines of different formats

| Construct | Dose (μg) | Deaths in group | Toxicity symptoms |
|---|---|---|---|
| IL-2(R38D/K43E/E61R)/PD-L2-Fc | 200 | None | Fur texture, reduced activity |
| immunocytokine | 1000 | None | Fur texture, reduced activity, |
| (IL-2 positioned at hinge region; | | | weight loss |
| construct #11) | | | |
| PD-L2-Fc/IL-2(R38D/K43E/E61R) | 200 | 5/5 | Fur texture, reduced activity, |
| immunocytokine | | | weight loss, morbidity |
| (IL-2 fused to the C′ of Fc) | 1000 | 5/5 | Fur texture, reduced activity, |
| | | | weight loss, morbidity |
| Parental PD-L2-hinge-Fc fusion | 1000 | None | None |
| protein (control) | | | |

### Example 18: In Vivo Efficacy of IL-12 Immunomodulatory Molecules in 4T1 Syngeneic Tumor Mice Model

### Construction of IL-12(E59A/F60A)/IL-2(R38D/K43E/E61R)/Anti-PD-1 Immunomodulatory Molecule

As described in Example 1, an anti-human PD-1 antibody comprising nivolumab (Opdivo®)) VH and VL was used as the parental full-length antibody. The IL-2 R38D/K43E/E61R variant (SEQ ID NO: 26) was positioned within the hinge region of one heavy chain of the heterodimeric anti-PD-1 antibody, and the single-chain IL-12 E59A/F60A variant (SEQ ID NO: 68) was positioned within the hinge region of the other heavy chain of the heterodimeric anti-PD-1 antibody, to construct the “IL-12(E59A/F60A)/IL-2(R38D/K43E/E61R)/anti-PD-1 immunomodulatory molecule” (“IW-#54” or “construct #54”). The linker within the single-chain IL-12(E59A/F60A) variant can also be changed to SEQ ID NO: 246, and the single-chain IL-12(E59A/F60A) variant can comprise SEQ ID NO: 254. The construct was expressed and purified as described in Example 1.

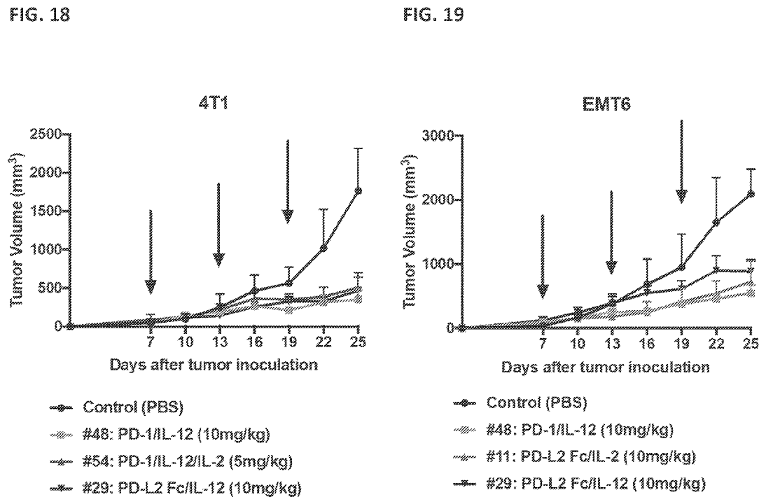

Mice (˜20g body weight) were inoculated with 0.25×10⁶ 4T1 murine breast cancer cells. Seven days after tumor inoculation, tumor size was measured to be about 50-150 mm³. After measuring tumor size, mice were injected with 10 mg/kg (˜200 μg) IL-12(E59A/F60A)/anti-PD-1 immunomodulatory molecule (constructed in Example 1; IW-#48), 10 mg/kg (˜200 μg) IL-12(E59A/F60A)/PD-L2-Fc immunomodulatory molecule (constructed in Example 2; IW-#29), 5 mg/kg (˜100 μg) IL-12(E59A/F60A)/IL-2(R38D/K43E/E61R)/anti-PD-1 immunomodulatory molecule (IW-#54), or PBS (negative control). A total of three injections (10 mg/kg or 5 mg/kg per injection, respectively) were given on days 7, 13, and 19 post-inoculation (indicated by black arrows in FIG. 18). Tumor size was measured every 3 days since the first injection. Mice were sacrificed once tumor size reached over 2000 mm³.

Breast cancer as reflected by 4T1 mice model is highly resistant to current immunotherapies, including anti-PD-1, anti-CTLA-4, and combination treatment with anti-PD-1 and anti-CTLA-4 antibodies. As can be seen from FIG. 18, all three IL-12-based immunomodulatory molecules significantly inhibited 4T1 tumor growth, demonstrating promising in vivo efficacy.

Further, IL-12(E59A/F60A)/anti-PD-1 (IW-#48) and IL-12(E59A/F60A)/PD-L2-Fc (IW-#29) showed similar cytotoxicity against 4T1 tumor when administered at the same dose (FIG. 18). In combination with results from Example 10, these data further demonstrate that compared to anti-PD-1 antigen-binding domain (non-agonist Ab) which blocks (or does not induce) PD-1 immunosuppression signal, using PD-L2 extracellular domain in the immunomodulatory molecule construct can not only achieve similar anti-tumor effect, but also reduce unwanted toxicity, likely by balancing the immunostimulating/pro-inflammatory activity of cytokines (e.g., IL-12) with an immunosuppression signal from PD-L2/PD-1 signaling.

### Example 19: In Vivo Efficacy of IL-12 Immunomodulatory Molecules in EMT6 Syngeneic Tumor Mice Model

Mice (˜20g body weight) were inoculated with 0.25×10⁶ EMT6 murine breast cancer cells. Seven days after tumor inoculation, tumor size was measured to be about 50-150 mm³. After measuring tumor size, mice were injected with 10 mg/kg (˜200 μg) IL-12(E59A/F60A)/anti-PD-1 immunomodulatory molecule (constructed in Example 1; IW-#48), 10 mg/kg (˜200 μg) IL-12(E59A/F60A)/PD-L2-Fc immunocytokine (constructed in Example 2; IW-#29), 10 mg/kg (˜200 μg) IL-2(R38D/K43E/E61R)/PD-L2-Fc immunocytokine (constructed in Example 11; IW-#11), or PBS (negative control). A total of three injections (10 mg/kg per injection) were given on days 7, 13, and 19 post-inoculation (indicated by black arrows in FIG. 19). Tumor size was measured every 3 days since the first injection. Mice were sacrificed once tumor size reached over 2000 mm³.

EMT6 tumor growth is resistant to anti-PD-1 immunotherapy. As can be seen from FIG. 19, all immunomodulatory molecules significantly inhibited EMT6 tumor growth, of which IL-12(E59A/F60A)/anti-PD-1 (IW-#48) and IL-2(R38D/K43E/E61R)/PD-L2-Fc (IW-#11) immunomodulatory molecules demonstrated better efficacy compared to IL-12(E59A/F60A)/PD-L2-Fc (IW-#29) immunomodulatory molecule. The slightly lower efficacy seen in PD-L2-Fc based IL-12 immunomodulatory molecule was likely due to stimulated PD-1 inhibitory immune checkpoint signaling upon PD-L2-PD-1 binding, which created an immunosuppression signal that “balances” against the immunostimulating/pro-inflammatory activity of IL-12.

### Example 20: Position of Cytokine or Variant Thereof within the Immunocytokine Affects Non-Specific Activities of the Immunocytokine

Two immunomodulatory molecule designs were generated to test whether placement of the cytokine or variant thereof at the hinge region (between antigen-binding domain and Fc fragment; hidden format) or at the C-terminus of the Fc fragment (e.g., C′ of antibody heavy chain; exposed format) could affect the targeted activity of the cytokine or variant thereof. The first design incorporated the cytokine at the hinge region of one heavy chain of an anti-PD-1 antibody (non-agonist): within the hinge region between CH1 and CH2 (the immunomodulatory molecules were named in the format of “IL-12/anti-PD-1”). The second design fused the cytokine to the C-terminus of one heavy chain of an anti-PD-1 antibody (non-agonist) through a linker (the immunomodulatory molecules were named in the format of “anti-PD-1/IL-12”), which is a common design among current immunocytokines.

IL-12(E59A/F60A)/anti-PD-1 (IW-#48 or construct #48), IL-12(E59A)/anti-PD-1 immunocytokine (IW-#46 or construct #46), and IL-12(G64A)/anti-PD-1 (IW-#47 or construct #47) with IL-12 variant (single-chain N′ to C′ IL-12B (p40 variant)-linker-IL-12A (wt p35)) positioned within the hinge region of one heavy chain of the heterodimeric anti-PD-1 antibody (nivolumab) were constructed as described in Example 1.

To make the heavy chain C′ cytokine fusion constructs, single-chain IL-12(E59A) variant (SEQ ID NO: 69), single-chain IL-12(G64A) variant (SEQ ID NO: 70), or single-chain IL-12(E59A/F60A) variant (SEQ ID NO: 68) was fused to the C′ of one heavy chain of the heterodimeric anti-PD-1 antibody (nivolumab) via a G/S containing peptide linker. The constructs are hereinafter referred to as anti-PD-1/IL-12(E59A) (construct #46HC′), anti-PD-1/IL-12(G64A) (construct #47HC′), and anti-PD-1/IL-12(E59A/F60A) (construct #48HC′), respectively. The heavy chain non-fusion polypeptide of the heterodimeric anti-PD-1 antibody has sequence of SEQ ID NO: 51. The linker within the single-chain IL-12 variant (e.g., single-chain IL-12(E59A/F60A) variant) can also be changed to SEQ ID NO: 246, for example, the single-chain IL-12(E59A/F60A) variant can comprise SEQ ID NO: 254.

IL-12 signal transduction assays using HEK-Blue™ IL-12 and HEK-PD-1-IL-12 (generated in-house by overexpressing human PD-1 in HEK-Blue™ IL-12 Cells using a lentiviral vector) cells were similarly conducted as described in Example 1 with two configurations of immunocytokines described above and rIL-12 (positive control).

TABLE 10
IL-12 biological activity of different IL-12immunocytokine formats

| | Location of | IL-12 mutation | | |
|---|---|---|---|---|
| Construct | cytokine | (in p40 subunit) | HEK-IL-12 | HEK-PD-1-IL-12 |
| rIL12 (control) | / | / | 100% | 100% |
| IL-12(G64A)/anti-PD-1 | Hinge of one | G64A | 90% | 230% |
| (construct #47) | heavy chain | | | |
| IL-12(E59A)/anti-PD-1 | | E59A | 5% | 78% |
| (construct #46) | | | | |
| IL-12(E59A/F60A)/anti-PD-1 | | E59A/F60A | <0.2% | 32% |
| (construct #48) | | | | |
| anti-PD-1/IL-12(G64A) | C-terminus | G64A | 85% | 210% |
| (construct #47HC′) | of one heavy | | | |
| anti-PD-1/IL-12(E59A) | chain | E59A | 15% | 86% |
| (construct #46HC′) | | | | |
| anti-PD-1/IL-12(E59A/F60A) | | E59A/F60A | 4% | 37% |
| (construct #48HC′) | | | | |

In HEK-IL-12 reporter assay, both IL-12 immunomodulatory molecule formats were only able to bind to HEK-IL-12 cells via IL-12 moiety/IL-12 receptor interaction, if the IL-12 moiety was accessible (e.g., heavy chain C′ fusion format). In HEK-PD-1-IL-12 reporter assay, both IL-12 immunomodulatory molecule formats were able to bind to HEK-PD-1-IL-12 cells via both IL-12 moiety/IL-12 receptor interaction, and anti-PD-1 antigen-binding fragment/PD-1 interaction.

As shown in Table 10, the hinge fusion design had significantly decreased non-specific activity (i.e., cytokine activity in the absence of PD-1 binding) compared to the heavy chain C-terminus fusion design. In PD-1 negative cells (HEK-IL-12), construct #48 showed almost undetectable levels of IL-12 activity (<0.2%), compared to 4% for construct #48HC′. Similar results were observed for construct #47 and construct #47HC′ (5% compared to 15%, respectively). The IL-12 double mutation E59A/F60A also significantly reduced non-specific activity compared to single mutation E59A or G64A. In PD-1 positive cells (HEK-PD-1-IL-12), IL-12 targeted activity was similar between the corresponding hinge fusion format and heavy chain C-terminus fusion format, suggesting that the hinge fusion design does not significantly inhibit IL-12 activity in the presence of antigen-positive cells (or antigen-binding). Taken together, the hinge placement of cytokine (especially certain cytokine variants) can greatly reduce non-specific IL-12 activity in the absence of binding of the antigen-binding domain.

### Example 21: Generation of IL-12/Anti-PD-1 Immunomodulatory Molecules with Reduced Affinity for PD-1

Generation of Anti-PD-1 Antibody Variants with Reduced PD-1 Binding Affinity

Due to nivolumab's high binding affinity for PD-1, IL-12/anti-PD-1 immunomodulatory molecules using wildtype nivolumab as parental antibody may direct IL-12 activity to all PD-1 positive cells, regardless of PD-1 expression levels. Targeting of such a large population of PD-1 positive cells could result in a cytokine storm or other adverse side effects, from activating any PD-1 positive immune cells (e.g., T cells) by the immunostimulatory cytokine moiety.

To generate anti-PD-1 mutants (non-agonist Ab) with reduced binding affinity for PD-1, so that it only targets high expressing PD-1 cells (e.g., T cells), mutations were introduced to HC-CDR3 at D100 or N99 positions of nivolumab: HC-CDR3(D100N), HC-CDR3(D100G), HC-CDR3(D100R), HC-CDR3(N99G), HC-CDR3(N99A) and HC-CDR3(N99M). The affinity of these anti-PD-1 antibodies (non-agonist Ab) were measured by Biacore and cell-based assays, calibrated by wildtype nivolumab binding affinity (see Table 11). “N/A” indicates non-detected PD-1 binding.

To construct anti-PD-1 heterodimer, one heavy chain comprises a hinge region comprising SEQ ID NO: 78, and an Fc domain subunit comprising SEQ ID NO: 97; the other heavy chain comprises a hinge region comprising SEQ ID NO: 77, and an Fc domain subunit comprising SEQ ID NO: 98. The two light chains each comprises the amino acid sequence of SEQ ID NO: 50.

TABLE 11
PD-1 binding affinities of various anti-PD-1 heavy chain
mutants (non-agonist)

| Heavy chain mutation | Affinity to PD-1 (Kd) |
|---|---|
| WT | 2.6 nM |
| D100N | 25 nM |
| D100G | 130 nM |
| D100R | 910 nM |
| N99G | 2300 nM |
| N99A | N/A |
| N99M | N/A |

Construction of IL-12/Anti-PD-1 Immunomodulatory Molecules with Reduced Affinity for PD-1

Various IL-12/anti-PD-1 immunomodulatory molecules were generated as described in Example 1 by placing single-chain IL-12(E59A/F60A) variant (SEQ ID NO: 68 or 254) within the hinge region of one heavy chain of the various heterodimeric anti-PD-1 mutants (non-agonist) described above. The sequence of the heavy chain cytokine fusion polypeptide is provided in Table 12 for each construct. The corresponding pairing non-fusion heavy chain comprises from N′ to C′ VH (with corresponding HC-CDR3 mutation)-CH1-hinge (SEQ ID NO: 77)-Fc domain subunit (SEQ ID NO: 98).

### IL-12 Signal Transduction Assay

IL-12 signal transduction assays were similarly conducted as described in Example 1 using IL-12/anti-PD-1(mut) immunomodulatory molecules with reduced PD-1 binding affinity (IL-12(E59A/F60A)/anti-PD-1(wt) and rIL-12 served as control), on HEK-Blue™ IL-12 cells and HEK-PD-1-IL-12 cells. Two variations of HEK-PD-1-IL-12 cells were used: one with high PD-1 expression “HEK-IL-12-PD-1(high)” (as described in Example 1, over-expressing PD-1), and one with 30-fold lower PD-1 expression “HEK-IL-12-PD-1(low)” (generated in-house by expressing lower amount of human PD-1 in HEK-Blue™ IL-12 Cells using a lentiviral vector). Cells were incubated with 20 ng/mL of the various IL-12/anti-PD-1 immunomodulatory molecules (or control) for 24 hours.

### IFN-γ Release Assay

IFN-γ release assays were similarly conducted as described in Example 13 using the IL-12(E59A/F60A)/anti-PD-1(mut) immunomodulatory molecules with reduced PD-1 binding affinity. IL-12(E59A/F60A)/anti-PD-1(wt) and rIL-12 served as control. Briefly, T cells were activated by incubating PBMCs with an anti-CD3 antibody (OKT3, 100 ng/mL) for three days. PBMCs were washed to remove the anti-CD3 antibody and incubated with 200 ng/mL of the various IL-12(E59A/F60A)/anti-PD-1(mut) immunomodulatory molecules (or control) for 24 hours. After one day, the amount of IFN-γ released into the cell culture medium was measured.

TABLE 12
IL-12 biological activity of IL-12(E59A/F60A)/anti-PD-1 immunomodulatory molecules
comprising various anti-PD-1 heavy chain mutations (reduced PD-1 binding affinity)

| Construct | Affinity | HEK | HEK-IL-12- | HEK-IL-12- | PBMC |
|---|---|---|---|---|---|
| (heavy chain fusion sequence) | PD-1 (Kd) | IL-12 | PD-1 (high) | PD-1 (low) | (IFN-γ ng/ml) |
| rIL-12 | | 100% | 100% | 100% | 2300 |
| IL-12(E59A/F60A)/anti-PD-1(WT) | 2.6 nM | <0.2% | 36% | 38% | 1400 |
| (construct IW-#48; SEQ ID NO: 22) | | | | | |
| IL-12(E59A/F60A)/anti-PD-1(D100N) | 25 nM | <0.2% | 38% | 33% | 820 |
| (SEQ ID NO: 149) | | | | | |
| IL-12(E59A/F60A)/anti-PD- | 130 nM | <0.2% | 33% | 17% | 120 |
| 1(D100G) | | | | | |
| (SEQ ID NO: 150) | | | | | |
| IL-12(E59A/F60A)/anti-PD- | 910 nM | <0.2% | 10% | 3% | 69 |
| 1(D100R) | | | | | |
| (SEQ ID NO: 151) | | | | | |
| IL-12(E59A/F60A)/anti-PD-1(N99G) | 2300 nM | <0.2% | 5% | <0.2% | 34 |
| (SEQ ID NO: 152) | | | | | |
| IL-12(E59A/F60A)/anti-PD-1(N99A) | N/A | <0.2% | 3% | <0.2% | 12 |
| (SEQ ID NO: 153) | | | | | |
| IL-12(E59A/F60A)/anti-PD-1(N99M) | N/A | <0.2% | <0.2% | <0.2% | 13 |
| (SEQ ID NO: 154) | | | | | |

As can be seen from Table 12, for all immunomodulatory molecules tested, no non-specific IL-12 activity was observed in the absence of anti-PD-1 binding (see HEK-IL-12 column). Their ability of transducing IL-12 signal in the presence of PD-1 binding, as well as their ability in inducing IFN-γ release, decreases as anti-PD-1 binding affinity decreases, demonstrating antigen-binding dependent cytokine activity of the hinge fusion design. IL-12(E59A/F60A)/anti-PD-1 immunomodulatory molecules with D100G, D100R, or N99G mutations in anti-PD-1 heavy chain showed notable differences in binding between high and low PD-1 expressing cells. These results indicate that cells expressing a higher level of PD-1 can be specifically targeted using IL-12(E59A/F60A)/anti-PD-1(mut) immunomodulatory molecules with reduced affinity for PD-1. IFN-γ secretion induced by these constructs were also much lower compared to IL-12/anti-PD-1(wt) immunomodulatory molecule and rIL-12 control.

Hence, IL-12(E59A/F60A)/anti-PD-1(mut) immunomodulatory molecules described herein, and maybe other immunomodulatory molecules constructed based on antigen-binding domain with reduced antigen binding affinity, may be used to specifically target cells of interest with high-antigen expression, with reduced off-target effect and/or cytokine storm.

### Example 22: Reducing PD-1 Binding Affinity in IL-12/Anti-PD-1 Immunomodulatory Molecules Reduces Toxicity in Mice

Humanized PD-1 mice (by inserting, within the mouse PD-1 locus, a chimeric PD-1 with a human extracellular domain, a murine transmembrane domain and a murine intracellular domain) derived from the C57 strain (5-6 weeks age, 20 g females) were injected with 10 mg/kg or 50 mg/kg (per injection) of various IL-12(E59A/F60A)/anti-PD-1(mut) immunomodulatory molecules described in Example 21. IL-12(E59A/F60A)/anti-PD-1(wt) immunomodulatory molecule (IW-#48 or construct #48) served as control. A total of four injections were given on Days 0, 4, 8, and 12. Mice were monitored daily for mortality and four toxicity symptoms: i) fur texture, ii) reduced activity, iii) morbidity, and iv) weight loss.

Mice injected with the IL-12(E59A/F60A)/anti-PD-1(wt) immunomodulatory molecule (IW-#48) comprising wildtype nivolumab showed the greatest toxicity, with all mice in the group dying after receiving either the second or third injection, even for lower dosing. In contrast, mice injected with IL-12(E59A/F60A)/anti-PD-1(mut) immunomodulatory molecules comprising anti-PD-1 with reduced PD-1 binding affinity showed reduced toxicity, with death observed only in the group treated with IL-12(E59A/F60A)/anti-PD-1(D100N). As can be seen from Table 13, the severity of toxicity symptom reduces as PD-1 binding affinity decreases, and/or as the dose decrease, among the constructs.

TABLE 13
In vivo toxicity of IL-12/anti-PD-1 immunomodulatory molecules

| | Affinity | Dose | | |
|---|---|---|---|---|
| Construct | PD-1 (Kd) | (mg/kg) | Toxicity Symptoms | Deaths |
| IL-12(E59A/F60A)/anti-PD-1(WT) | 2.6 nM | 10 | Fur texture, reduced activity, | 5/5 |
| (construct IW-#48) | | | weight loss, morbidity | |
| | | 50 | Fur texture, reduced activity, | 5/5 |
| | | | weight loss, morbidity | |
| IL-12(E59A/F60A)/anti-PD- | 25 nM | 10 | Fur texture, reduced activity, | 3/5 |
| 1(D100N) | | | weight loss, morbidity | |
| | | 50 | Fur texture, reduced activity, | 5/5 |
| | | | weight loss, morbidity | |
| IL-12(E59A/F60A)/anti-PD- | 130 nM | 10 | Fur texture, reduced activity | None |
| 1(D100G) | | 50 | Fur texture, reduced activity | None |
| IL-12(E59A/F60A)/anti-PD-1(D100R) | 910 nM | 10 | Fur texture (moderate) | None |
| | | 50 | Fur texture, reduced activity | None |
| IL-12(E59A/F60A)/anti-PD-1(N99G) | 2300 nM | 10 | None | None |
| | | 50 | Fur texture (moderate) | None |
| IL-12(E59A/F60A)/anti-PD-1(N99M) | N/A | 10 | None | None |
| | | 50 | Fur texture (moderate) | None |

Due to wildtype nivolumab's (non-agonist) high binding affinity to PD-1 (K_{d}≈10⁻⁸-10⁻⁹ M), IL-12(E59/F60A)/anti-PD-1(WT) most likely binds and stimulates (via the cytokine activity) any PD-1 positive cell. This would include activated T-cells and NK cells, which would result in cytokine release syndrome. In contrast, IL-12/anti-PD-1 based immunomodulatory molecules with reduced binding affinity to hPD-1 can only bind a smaller population of PD-1 positive cells, particularly cells with very high PD-1 expression levels, such as exhausted T-cells. The data shown here is consistent with the data from the in vitro PBMC IFN-γ release assay in Example 21. These findings indicate that reducing the PD-1 binding affinity of anti-PD-1 antigen-binding domain (non-agonist anti-PD-1) to a K_{d} of between about 10⁻⁶-10⁻⁷ M (see, e.g., D100G, D100R, N99G in anti-PD-1 heavy chain) can greatly improve the safety of IL-12/anti-PD-1 immunomodulatory molecules, while retaining therapeutic efficacy.

### Example 23: Increasing the Binding Affinity of PD-L1 and PD-L2 does not Increase Toxicity of IL-12/PD-L1-Fc and IL-12/PD-L2-Fc Immunomodulatory Molecules

As shown in Examples 10, 18, and 19, replacing anti-PD-1 antigen-binding fragment (not agonist Ab) with PD-L2 extracellular domain in IL-12-based immunomodulatory molecules can significantly reduce toxicity, likely due to stimulated PD-1 inhibitory immune checkpoint signaling upon PD-L2-PD-1 binding, which created an immunosuppression signal that “balances” against the immunostimulating/pro-inflammatory activity of IL-12. To investigate if safety profiles of these “balancing” constructs can be further improved, IL-12 immunomodulatory molecules comprising PD-L1 or PD-L2 extracellular domain with increased PD-1 binding affinity were constructed, in order to enhance PD-1 immunosuppression signal.

Generation of PD-L1 Variants with Increased PD-1 Binding Affinity

Wildtype PD-L1 has a binding affinity for PD-1 of about 10⁻⁵-10⁻⁶ M, which is lower than that of nivolumab (K_{d}≈10⁻⁸-10⁻⁹ M). To increase the affinity for PD-1, PD-L1 mutants were generated. Mutations were introduced into the extracellular domain of wildtype PD-L1 with amino acid positions relative to SEQ ID NO; 120. These mutant PD-L1 extracellular domains were then fused to an Fc fragment via a hinge region to construct parental PD-L I-Fc constructs. To construct PD-L1-Fc heterodimer, one polypeptide chain comprises a hinge region comprising SEQ ID NO: 88, and an Fc domain subunit comprising SEQ ID NO: 97; the other polypeptide chain comprises a hinge region comprising SEQ ID NO: 87, and an Fc domain subunit comprising SEQ ID NO: 98. Mutation constructs were named in the format of PD-L1(mut)-Fc.

A description of the mutations made and PD-1 binding affinities (measured in PD-L1-Fc format) are shown in Table 14. Binding affinity for each PD-L1(mut)-Fc was calibrated based on PD-L1(wt)-Fc binding affinity. N/A indicates non-detectable PD-1 binding. These results indicate that all PD-L1(mut) achieved about 4-60 fold increase in PD-1 binding affinity compared to wildtype PD-L1. Among these, PD-L1(I54Q/E58M/R113T/M115L/S117A/G119K) (PD-L1(mut2)), PD-L1(I54Q/E58M/R113T/M115L/G119K) (PD-L1(mut6)), and PD-L1(I54Q/E58M/R113T/M115L/S117A) (PD-L1(mut7)) showed the highest fold increase in affinity for PD-1 as compared to wildtype PD-L1.

TABLE 14
PD-1 binding affinities of various PD-L1 mutants

| | Affinity (Kd) | Affinity (Kd) |
|---|---|---|
| PD-L1 mutations | human PD-1 | mouse PD-1 |
| None | 7500 nM | 5100 nM |
| (PD-L1(WT); SEQ ID NO: 121) | | |
| E58M/R113T/M115L/S117A/G119K | N/A | N/A |
| (PD-L1(mut1); SEQ ID NO: 122) | | |
| I54Q/E58M/R113T/M115L/S117A/G119K | 150 nM | 120 nM |
| (PD-L1(mut2); SEQ ID NO: 123) | | |
| I54Q/R113T/M115L/S117A/G119K | 910 nM | 820 nM |
| (PD-L1(mut3); SEQ ID NO: 124) | | |
| I54Q/E58M/M115L/S117A/G119K | 1090 nM | 980 nM |
| (PD-L1(mut4); SEQ ID NO: 125) | | |
| I54Q/E58M/R113T/S117A/G119K | 1203 nM | 1100 nM |
| (PD-L1(mut5); SEQ ID NO: 126) | | |
| I54Q/E58M/R113T/M115L/G119K | 555 nM | 420 nM |
| (PD-L1(mut6); SEQ ID NO: 127) | | |
| 154Q/E58M/R113T/M115L/S117A | 98 nM | 110 nM |
| (PD-L1(mut7); SEQ ID NO: 128) | | |

Generation of PD-L2 Variants with Increased PD-1 Binding Affinity

PD-L2 has a binding affinity for PD-1 of about 10⁻⁶-10⁻⁷ M, which is lower than that of nivolumab (K_{d}≈10⁻⁸-10⁻⁹ M). To increase the affinity for PD-1, PD-L2 mutants were generated. Mutations were introduced into the extracellular domain of wildtype PD-L2 with amino acid positions relative to SEQ ID NO: 105. These mutant PD-L2 extracellular domains were then fused to an Fc fragment via a hinge region to construct parental PD-L2-Fc constructs. To construct PD-L2-Fc heterodimer, one polypeptide chain comprises a hinge region comprising SEQ ID NO: 88, and an Fc domain subunit comprising SEQ ID NO: 97; the other polypeptide chain comprises a hinge region comprising SEQ ID NO: 87, and an Fc domain subunit comprising SEQ ID NO: 98. Mutation constructs were named in the format of PD-L2(mut)-Fc.

A description of the mutations made and PD-1 binding affinities (measured in PD-L2-Fc format) are shown in Table 15. Binding affinity for each PD-L2(mut)-Fc was calibrated based on PD-L2(wt)-Fc binding affinity. These results indicate that all PD-L2(mut) achieved about 2-5 fold increase in PD-1 binding affinity compared to wildtype PD-L2. Among these, PD-L2(S58V) (PD-L2(mut2)) and PD-L2(T56V/S58V/Q60L) (PD-L2(mut4)) showed the highest fold increase in affinity for PD-1 as compared to wildtype PD-L2.

TABLE 15
PD-1 binding affinities of various PD-L2 mutants

| | Affinity (Kd) | Affinity (Kd) |
|---|---|---|
| PD-L2 Mutations | human PD-1 | mouse PD-1 |
| None | 1200 nM | 980 nM |
| (PD-L2(WT); SEQ ID NO: 106) | | |
| T56V | 520 nM | 430 nM |
| (PD-L2(mut1); SEQ ID NO: 107) | | |
| S58V | 350 nM | 230 nM |
| (PD-L2(mut2); SEQ ID NO: 108) | | |
| Q60L | 490 nM | 320 nM |
| (PD-L2(mut3); SEQ ID NO: 109) | | |
| T56V/S58V/Q60L | 255 nM | 220 nM |
| (PD-L2(mut4); SEQ ID NO: 110) | | |

Construction of IL-12/PD-L1-Fc and IL-12/PD-L2-Fc Immunomodulatory Molecules with Increased Affinity for PD-1

Similarly as described in Example 10, heterodimeric PD-L1(mut)-Fc or PD-L2(mut)-Fc generated herein were used as parental antigen-binding proteins to construct IL-12 immunomodulatory molecules that bind PD-1. Single chain IL-12(E59A/F60A) variant (e.g., SEQ ID NO: 68 or 254) was placed at the N′ of the hinge of one polypeptide chain within the parental PD-L1(mut)-Fc or PD-L2(mut)-Fc heterodimers.

IL-12(E59A/F60A)/PD-L2(wt)-Fc immunomodulatory molecule (“construct #29” or “IW-#29”) was constructed as in Example 2 with wildtype PD-L2 extracellular domain. IL-12(E59A/F60A)/PD-L2(mut)-Fc immunocytokine comprises one IL-12 fusion polypeptide (from N′ to C′: PD-L2(mut) extracellular domain-GGGGSGGG linker (SEQ ID NO: 244)-single chain IL-12(E59A/F60A) variant (e.g., SEQ ID NO: 68 or 254)-GGGGSGGG linker (SEQ ID NO: 244)-hinge (SEQ ID NO: 88)-Fc domain subunit (SEQ ID NO: 97)); and one pairing polypeptide (from N′ to C′: PD-L2(mut) extracellular domain-GGGGSGGG linker (SEQ ID NO: 244)-hinge (SEQ ID NO: 87)-Fc domain subunit (SEQ ID NO: 98)). Exemplary IL-12 cytokine fusion chain of IL-12(E59A/F60A)/PD-L2(mut)-Fc immunomodulatory molecules can comprise SEQ ID NO: 167 or 168.

IL-12(E59A/F60A)/PD-L1(wt)-Fc immunomodulatory molecule comprises one IL-12 fusion polypeptide (from N′ to C′: PD-L1(wt) extracellular domain (SEQ ID NO: 121)-GGGGSGGG linker (SEQ ID NO: 244)-single chain IL-12(E59A/F60A) variant-GGGGSGGG linker (SEQ ID NO: 244)-hinge (SEQ ID NO: 88)-Fc domain subunit (SEQ ID NO: 97)); and one pairing polypeptide (from N′ to C′: PD-L1(wt) extracellular domain (SEQ ID NO: 121)-GGGGSGGG linker (SEQ ID NO: 244)-hinge (SEQ ID NO: 87)-Fc domain subunit (SEQ ID NO: 98)). IL-12(E59A/F60A)/PD-L1(mut)-Fc immunomodulatory molecule comprises one IL-12 fusion polypeptide (from N′ to C′: PD-L1(mut) extracellular domain (e.g., SEQ ID NO: 129)-GGGGSGGG linker (SEQ ID NO: 244)-single chain IL-12(E59A/F60A) variant-GGGGSGGG linker (SEQ ID NO: 244)-hinge (SEQ ID NO: 88)-Fc domain subunit (SEQ ID NO: 97)); and one pairing polypeptide (from N′ to C′: PD-L1(mut) extracellular domain-GGGGSGGG linker (SEQ ID NO: 244)-hinge (SEQ ID NO: 87)-Fc domain subunit (SEQ ID NO: 98)). The linkers can be changed to other linkers (e.g., GSG linker; SEQ ID NO: 203) or can be optional. Exemplary IL-12 cytokine fusion chain of IL-12(E59A/F60A)/PD-L1(mut)-Fc immunomodulatory molecules can comprise SEQ ID NO: 155 or 156.

To test the safety profiles of the IL-12-based immunomodulatory molecules constructed with increased affinity to PD-1, wildtype C57 mice (5-6 weeks age, 20 g females) were injected with 10 mg/kg or 50 mg/kg (per injection) of IL-12(E59A/F60A)/PD-L1(mut2)-Fc immunomodulatory molecule or IL-12(E59A/F60A)/PD-L2(mut2)-Fc immunomodulatory molecule, as these two constructs showed similar PD-1 binding affinity to both human and mouse PD-1 (˜10⁻⁷ M). IL-12(E59A/F60A)/PD-L1(wt)-Fc immunomodulatory molecule or IL-12(E59A/F60A)/PD-L2(wt)-Fc immunomodulatory molecule served as control. A total of four injections were given on Days 0, 4, 8, and 12. Mice were monitored daily for mortality and four toxicity symptoms: i) fur texture, ii) reduced activity, iii) morbidity, and iv) weight loss.

As can be seen from Table 16, increasing binding affinity to PD-1 does not significantly affect the safety profiles of IL-12(E59A/F60A)/PD-L1-Fc immunomodulatory molecule or IL-12(E59A/F60A)/PD-L2-Fc immunomodulatory molecule. These results show that IL-12 immunomodulatory molecules comprising mutant versions of PD-L1 and PD-L2 with increased binding affinities to PD-1 retain the safety profile of wildtype IL-12/PD-L1-Fc and IL-12/PD-L2-Fc immunomodulatory molecules. This may be applied to other PD-L1-Fc or PD-L2-Fc based immunomodulatory molecules as well, to construct other immunomodulatory molecules (e.g., IL-2 immunomodulatory molecules).

TABLE 16
In vivo toxicity of IL-12/PD-L2-Fc immunomodulatory molecules

| Construct | Affinity | Dose | Toxicity | Deaths in |
|---|---|---|---|---|
| (cytokine fusion chain sequence) | (Kd) mPD-1 | (mg/kg) | Symptoms | group |
| IL-12(E59A/F60A)/PD-L1(WT)-Fc | 5100 nM | 10 | None | None |
| | | 50 | None | None |
| IL-12(E59A/F60A)/PD-L1 | 120 nM | 10 | None | None |
| (I54Q/E58M/R113T/M115L/S117A/G119K)-Fc | | 50 | Fur texture | None |
| (“IL-12(E59A/F60A)/PD-L1 | | | (moderate) | |
| (mut2)-Fc”; SEQ ID NO: 155) | | | | |
| IL-12(E59A/F60A)/PD-L2(WT)-Fc | 980 nM | 10 | None | None |
| (construct IW-#29; SEQ ID NO: 17) | | 50 | None | None |
| IL-12(E59A/F60A)/PD-L2(S58V)-Fc | 230 nM | 10 | None | None |
| (IL-12(E59A/F60A)/PD-L2(mut2)-Fc; SEQ | | 50 | None | None |
| ID NO: 167) | | | | |

### Example 24: Generation of IL-2/PD-L1-Fc Immunomodulatory Molecules with IL-2 Biological Activity Directed to PD-1-Positive Cells

Certain cytokines have synergistic action, such as IL-12 and IL-2, IL-12 and IFN-γ. To reduce toxicity of IL-2 and immunomodulatory molecules thereof, two sets of IL-2 mutations were generated: mutations within IL-2 domain that interacts with IL2Ra (CD25) (R38D/K43E/E61R; SEQ ID NO: 26), and mutations within IL-2 domain that interacts with IL2Ry (CD132) (L18R, Q22E, Q126T, S130R, or any combinations thereof). See Table 17.

Heterodimeric PD-L1(mut2)-Fc immunomodulatory molecule was used as the parental PD-1 binding protein. First polypeptide chain comprises SEQ ID NO: 132 (N′ to C′: PD-L1(mut2) extracellular domain (SEQ ID NO: 123)-GGGGSGGG linker (SEQ ID NO: 244)-hinge (SEQ ID NO: 88)-Fc domain subunit (SEQ ID NO: 97)), second polypeptide chain comprises SEQ ID NO: 134 (N′ to C′: PD-L1(mut2) extracellular domain (SEQ ID NO: 123)-GGGGSGGG linker (SEQ ID NO: 244)-hinge (SEQ ID NO: 87)-Fc domain subunit (SEQ ID NO: 98)). To construct IL-2 immunomodulatory molecules, IL-2 variant was placed between the PD-L1(mut2) extracellular domain and the hinge. Briefly, IL-2(mut)/PD-L1(mut2)-Fc immunocytokine comprises one IL-2 fusion polypeptide (from N′ to C′: PD-L1(mut2) extracellular domain (SEQ ID NO: 123)-GGGSG linker (SEQ ID NO: 209)-IL-2(mut) variant-GGGGSGGG linker (SEQ ID NO: 244)-hinge (SEQ ID NO: 87)-Fc domain subunit (SEQ TD NO: 98)); and one pairing polypeptide SEQ ID NO: 132.

PD-L1(mut)-Fc immunomodulatory molecules comprising other PD-L1(mut) extracellular domain and/or cytokine moiety (e.g., other IL-2 variants) can be similarly constructed. For example, PD-L1(mut7)-Fc immunomodulatory molecules can be constructed by replacing PD-L1(mut2) extracellular domain with PD-L1(mut7) extracellular domain (SEQ ID NO: 128). Parental heterodimeric PD-L1(mut7)-Fc immunomodulatory molecule can comprising one chain of SEQ ID NO: 133, and the other chain of SEQ ID NO: 135. Exemplary IL-2 cytokine fusion chain of IL-2(mut)/PD-L1(mut7)-Fc immunomodulatory molecules can comprise any of SEQ ID NOs: 163-166, the pairing non-cytokine fusion chain can comprise 133.

HEK-Blue™ IL-2 Cells and HEK-PD-1-IL-2 cells were used to assess TL-2 signal activation activity of the constructs, as described in Example 12. As can be seen from Table 17, IL-2(R38D/K43E/E61R)/PD-L1(mut2)-Fc with IL-2 mutations only in CD25 binding domain (R38D/K43E/E61R) (see construct comprising SEQ ID NO: 179 chain) still retained about 16% IL-2 activity based on HEK-IL-2 assay (no PD-1 binding), while IL-2 immunomodulatory molecules further carrying IL-2 mutations in the CD132 binding domain significantly decreased IL-2 activity based on HEK-IL-2 assay, in the absence of PD-1 binding. Notably, IL-2 activity of some of the IL-2 immunomodulatory molecules further carrying CD132 binding domain mutations (see constructs comprising SEQ ID NO: 159, SEQ ID NO: 160, or SEQ ID NO: 161 chain) can be partially rescued when binding to PD-1 (based on HEK-IL-2-PD-1 assay). S130 may be crucial for IL-2 activity, as IL-2 immunomodulatory molecule further carrying S130R mutation in CD132 binding domain (see construct comprising SEQ ID NO: 162 chain), in combination with other IL-2 mutations, failed to exhibit any IL-2 activity even in the presence of PD-1 binding.

TABLE 17
IL-2 biological activity of IL-2/PD-L1-Fc immunomodulatory molecules

| | Non-fusion | IL-2 fusion or | | | | | |
|---|---|---|---|---|---|---|---|
| | polypeptide | non-fusion | IL-2 | CD25 | CD132 | | |
| | PD- | polypeptide PD- | SEQ | binding | binding | | HEK- |
| | L1(mut2)-Fc | L1(mut2)-Fc | ID | site | site | HEK- | IL-2- |
| Construct | SEQ ID NO: | SEQ ID NO: | NO: | mutations | mutations | IL-2 | PD-1 |
| rIL2 | / | / | | / | / | 100% | 100% |
| PD-L1(mut2)-Fc | 132 | 134 | / | / | / | <0.1% | <0.1% |
| IL-2(R38D/ | 132 | 179 | 26 | R38D/ | / | 16% | 76% |
| K43E/E61R)/PD- | | | | K43E/ | | | |
| L1(mut2)-Fc | | | | E61R | | | |
| IL-2(L18R/Q22E/ | 132 | 159 | 27 | R38D/ | L18R/ | 4% | 35% |
| R38D/K43E/E61R)/ | | | | K43E/ | Q22E | | |
| PD-L1(mut2)-Fc | | | | E61R | | | |
| IL-2(R38D/K43E/ | 132 | 160 | 28 | R38D/ | Q126T | 2% | 20% |
| E61R/Q126T)/ | | | | K43E/ | | | |
| PD-L1(mut2)-Fc | | | | E61R | | | |
| IL-2(L18R/Q22E/ | 132 | 161 | 29 | R38D/ | L18R/ | <0.1% | 5% |
| R38D/K43E/E61R/ | | | | K43E/ | Q22E/ | | |
| Q126T)/PD- | | | | E61R | Q126T | | |
| L1(mut2)-Fc | | | | | | | |
| IL-2(L18R/Q22E/ | 132 | 162 | 30 | R38D/ | L18R/ | <0.1% | <0.1% |
| R38D/K43E/E61R/ | | | | K43E/ | Q22E/ | | |
| Q126T/S130R)/ | | | | E61R | Q126T/ | | |
| PD-L1(mut2)-Fc | | | | | S130R | | |

### Example 25: Generation of IL-2/IL-12/PD-L1 Immunomodulatory Molecules with IL-2 and IL-12 Biological Activity Directed to PD-1-Positive Cells

Certain cytokines have synergistic action, such as IL-12 and IL-2. As shown in Examples above, IL-12(E59A/F60A)/PD-L1-Fc immunomodulatory molecule (hinge region) showed PD-1 binding dependent IL-12 activity. To investigate whether immunomodulatory molecules can be constructed with synergistic IL-12 and IL-2 activity, while retaining PD-1 binding dependent cytokine activity, different configurations of immunomodulatory molecules were constructed. Heterodimeric PD-L1(mut2)-Fc was used as parental PD-1 binding fusion protein (constructed in Example 23). Set I: one polypeptide chain comprises single chain IL-12(E59A/F60A) polypeptide positioned at the hinge region of PD-L1(mut2)-Fc (see SEQ ID NO: 155 constructed in Example 23); the pairing polypeptide chain does not comprise IL-2 moiety (control; SEQ ID NO: 134 constructed in Examples 23 and 24), or comprises IL-2 variant (either with L18R/Q22E/R38D/K43E/E61R mutation (SEQ ID NO: 27), or with R38D/K43E/E61R/Q126T mutation (SEQ ID NO: 28)) positioned at the hinge region of PD-L1(mut2)-Fc. These immunomodulatory molecules are named in the format of IL-2/IL-12(E59A/F60A)/PD-L1(mut2)-Fc. See FIG. 14A for exemplary structure. Set II: one polypeptide chain comprises IL-12(E59A/F60A) fused to the C′ of PD-L1(mut2)-Fc via GGGGSGGG linker (see SEQ ID NO: 157); the pairing polypeptide chain does not comprise IL-2 moiety (control; SEQ ID NO: 134 constructed in Examples 23 and 24), or comprises TL-2 variant (either with L18R/Q22E/R38D/K43E/E61R mutation (SEQ ID NO: 27), or with R38D/K43E/E61R/Q126T mutation (SEQ ID NO: 28)) positioned at the hinge region of PD-L1(mut2)-Fc. These immunomodulatory molecules are named in the format of IL-2/PD-L1(mut2)-Fc/IL-12(E59A/F60A), indicating that IL-12 moiety is at the C′ of Fc. See FIG. 15A for exemplary structure. See Table 18 for construct sequences.

HEK-Blue™ IL-2 Cells and HEK-PD-1-IL-2 cells were used to assess IL-2 signal activation activity of the constructs, as described in Example 12. HEK-Blue™ IL-12 Cells and HEK-PD-1-IL-12 cells were used to assess IL-12 signal activation activity of the constructs, as described in Example 1.

As can be seen from Table 18, IL-12(E59A/F60A)/PD-L1(mut2)-Fc (hinge fusion) did not have detectable IL-12 activity in the absence of PD-1 binding, while PD-1 binding rescued the IL-12 activity to 24%. When IL-12(E59A/F60A) was placed at C′ of Fc as PD-L1(mut2)-Fc/IL-12(E59A/F60A), IL-12 activity was about 1%-2% in the absence of PD-1 binding, and IL-12 activity was further rescued by PD-1 binding (˜25%), to similar extent as the IL-12 hinge fusion.

By adding on L-2 in the pairing chain at the hinge region, for both IL-12 hinge fusion and C′ fusion formats, IL-2 activity was about 2% H4% in the absence of PD-1 binding, but was rescued to about 20%-35% by PD-1 binding.

These data indicate that IL-12 and IL-2 can both retain PD-1-binding dependent activity when constructed in trispecific immunomodulatory molecule format. Further, IL-12 and IL-2 moieties did not have significant negative impact on each other's activity.

TABLE 18
IL-2 and IL-12 biological activity of IL-2/IL-12/PD-L1-Fc immunomodulatory
molecules and IL-2/PD-L1-Fc/IL-12 immunomodulatory molecules

| | | IL-2 fusion or | | | | |
|---|---|---|---|---|---|---|
| | IL-12 fusion | non-fusion | | HEK- | | HEK- |
| | polypeptide | polypeptide | | IL-2- | HEK- | IL-12- |
| Construct | SEQ ID NO: | SEQ ID NO: | HEK-IL-2 | PD-1 | IL-12 | PD-1 |
| rIL-2 | / | / | 100% | 100% | / | / |
| rIL-12 | / | / | / | / | 100% | 100% |
| IL-12(E59A/F60A)/PD- | 155 | 134 | <0.1% | <0.1% | <0.2% | 24% |
| L1(mut2)-Fc | (IL-12 hinge) | (no IL-2) | | | | |
| IL-2(L18R/Q22E/ | 155 | 159 | 4% | 35% | <0.2% | 19% |
| R38D/K43E/E61R)/ | (IL-12 hinge) | | | | | |
| IL-12(E59A/F60A)/ | | | | | | |
| PD-L1(mut2)-Fc | | | | | | |
| IL-2(R38D/K43E/ | 155 | 160 | 2% | 20% | <0.2% | 25% |
| E61R/Q126T)/ | (IL-12 hinge) | | | | | |
| IL-12(E59A/F60A)/ | | | | | | |
| PD-L1(mut2)-Fc | | | | | | |
| PD-L1(mut2)-Fc/IL- | 157 | 134 | <0.1% | <0.1% | 1% | 25% |
| 12(E59A/F60A) | (IL-12 at C′) | (no IL-2) | | | | |
| IL-2(L18R/Q22E/ | 157 | 159 | 3% | 32% | 2% | 27% |
| R38D/K43E/E61R)/ | (IL-12 at C′) | | | | | |
| PD-L1(mut2)-Fc/ | | | | | | |
| IL-12(E59A/F60A) | | | | | | |
| IL-2(R38D/K43E/ | 157 | 160 | 3% | 31% | 1% | 30% |
| E61R/Q126T)/ | (IL-12 at C′) | | | | | |
| PD-L1(mut2)-Fc/IL- | | | | | | |
| 12(E59A/F60A) | | | | | | |

### Example 26: Placing IL-12 Moiety at the Hinge Region can Greatly Improve Safety Profiles of IL-12/PD-L1-Fc Immunomodulatory Molecules and IL-2/IL-12/PD-L1-Fc Immunomodulatory Molecules

PD-L1(mut2)-Fc constructed in Examples 23 and 24 was used as parental PD-1 binding fusion protein, as it showed similar PD-1 binding affinity in both human and mice.

To test safety profiles in vivo, a mouse single-chain IL-12 variant (SEQ ID NO: 72) with E59A/F60A mutations in the p40 subunit and a p35 wildtype subunit was similarly constructed as described herein: from N′ to C′ p40(E59A/F60A)-GGPGGGGSGGGSGGGG linker (SEQ ID NO: 245)-p35(wt). Two sets of IL-12 fusion polypeptides were constructed, similar to Example 25. Set I: one polypeptide chain comprises single chain mIL-12(E59A/F60A) polypeptide positioned at the hinge region of PD-L1(mut2)-Fc (see SEQ ID NO: 180); the pairing polypeptide chain does not comprise IL-2 moiety (control; SEQ ID NO: 134 constructed in Examples 23 and 24), or comprises IL-2 variant (with R38D/K43E/E61R mutation (SEQ ID NO: 26), L18R/Q22E/R38D/K43E/E61R mutation (SEQ ID NO: 27), or with R38D/K43E/E61R/Q126T mutation (SEQ ID NO: 28)) positioned at the hinge region of PD-L1(mut2)-Fc. These immunomodulatory molecules are named in the format of IL-2/IL-12(E59A/F60A)/PD-L1(mut2)-Fc. See FIG. 14A for exemplary structure. Set II: one polypeptide chain comprises single-chain mIL-12(E59A/F60A) fused to the C′ of PD-L1(mut2)-Fc via GGGGSGGG linker (see SEQ TD NO: 157); the pairing polypeptide chain does not comprise IL-2 moiety (control; SEQ ID NO: 134 constructed in Examples 23 and 24), or comprises IL-2 variant (with R38D/K43E/E61R mutation (SEQ ID NO: 26), with L18R/Q22E/R38D/K43E/E61R mutation (SEQ ID NO: 27), or with R38D/K43E/E61R/Q126T mutation (SEQ ID NO: 28)) positioned at the hinge region of PD-L1(mut2)-Fc. These immunomodulatory molecules are named in the format of IL-2/PD-L1(mut2)-Fc/IL-12(E59A/F60A), indicating that IL-12 moiety is at the C′ of Fc. See FIG. 15A for exemplary structure. See Table 7 for construct sequences. A Control Set did not have any IL-12 moiety fusion to PD-L1(mut2)-Fc (SEQ ID NO: 132).

To test safety profiles of these constructs, wild-type C57 mice (5-6 weeks age, weight 20g, female) were injected with PBS (control), or the immunomodulatory molecules (10 mg/kg per injection) described herein. A total of 4 injections were given every 4 days. Mice were monitored for death and toxicity symptoms, such as fur texture, reduced activity, and weight loss. 48 hours after the 2^{nd} injection, blood was collected and serum concentrations of IFN-γ was measured.

As shown in Table 19, immunomodulatory molecules comprising IL-2 additional mutations in CD132 binding domain (L18R/Q22E, or Q126T) in addition to R38D/K43E/E61R in CD25 binding domain, showed much greater safety profiles compared to those without CD132 binding domain mutations (see constructs comprising SEQ ID NO: 179 chain), irrespective of if the IL-12 moiety is at C′ or at hinge.

As shown in Table 19, immunomodulatory molecules with IL-12 at the C′ of Fc, IL-2(mut)/PD-L1(mut2)-Fc/mIL-12(E59A/F60A) showed higher toxicity compared to IL-12 positioned at hinge region (IL-2(mut)/mIL-12(E59A/F60A)/PD-L1(mut2)-Fc). IL-2(mut)/PD-L1(mut2)-Fc/mIL-12(E59A/F60A) also induced much higher (20-30 folds) cytokine release (see IFN-γ level) compared to IL-12 hinge fusion design.

Taken together, our in vivo and in vitro data presented herein suggested that immunomodulatory molecules with cytokine (e.g., immunostimulatory cytokines such as IL-12 or variant thereof) positioned at the hinge region can significantly improve the safety profile, even when more cytokines with synergistic actions are present in the same construct (e.g., IL-2/IL-12/PD-L1-Fc). Mutations in cytokines to reduce their immunostimulatory activities, and/or mutations in antigen-binding domain (e.g., anti-PD-1 or PD-L1, or PD-L2), can further improve safety and/or therapeutic efficacy of the constructs.

TABLE 19
IL-2 and IL-12 biological activity of IL-2/IL-12/PD-L1-Fc immunomodulatory molecules
and IL-2/PD-L1-Fc/IL-12 immunomodulatory molecules

| | IL-12 fusion | IL-2 fusion or | | | |
|---|---|---|---|---|---|
| | or non-fusion | non-fusion | | Deaths | |
| | polypeptide | polypeptide | Toxicity | in | Blood |
| Construct | SEQ ID NO: | SEQ ID NO: | Symptoms | group | IFN-γ |
| PBS | / | / | None | None | 5 pg/ml |
| PD-L1(mut2)-Fc | 132 | 134 | None | None | 6 pg/ml |
| | (no IL-12) | (no IL-2) | | | |
| IL-2(R38D/K43E/E61R)/PD- | | 179 | Fur texture, | None | 45 pg/ml |
| L1(mut2)-Fc | | | reduced activity | | |
| IL- | | 159 | None | None | 32 pg/ml |
| 2(L18R/Q22E/R38D/K43E/E61R)/ | | | | | |
| PD-L1(mut2)-Fc | | | | | |
| IL- | | 160 | None | None | 24 pg/ml |
| 2(R38D/K43E/E61R/Q126T)/PD- | | | | | |
| L1(mut2)-Fc | | | | | |
| mIL-12(E59A/F60A)/PD- | 180(mIL-12 at | 134 | None | None | 80 pg/ml |
| L1(mut2)-Fc | hinge) | (no IL-2) | | | |
| IL-2(R38D/K43E/E61R)/mIL- | | 179 | Fur texture, | None | 90 pg/ml |
| 12(E59A/F60A)/PD-L1(mut2)-Fc | | | reduced activity | | |
| IL- | | 159 | None | None | 64 pg/ml |
| 2(L18R/Q22E/R38D/K43E/E61R)/ | | | | | |
| mIL-12(E59A/F60A)/PD- | | | | | |
| L1(mut2)-Fc | | | | | |
| IL- | | 160 | None | None | 45 pg/ml |
| 2(R38D/K43E/E61R/Q126T)/mIL- | | | | | |
| 12(E59A/F60A)/PD-L1(mut2)-Fc | | | | | |
| PD-L1(mut2)-Fc/mIL- | 181 | 134 | Fur texture | None | 1400 pg/ml |
| 12(E59A/F60A) | (mIL-12 at C′) | (no IL-2) | (moderate) | | |
| IL-2(R38D/K43E/E61R)/PD- | | 179 | Fur texture, | 1/5 | 5600 pg/ml |
| L1(mut2)-Fc/mIL-12(E59A/F60A) | | | reduced activity | | |
| IL- | | 159 | Fur texture, | None | 1800 pg/ml |
| 2(L18R/Q22E/R38D/K43E/E61R)/ | | | reduced activity | | |
| PD-L1(mut2)-Fc/mIL- | | | | | |
| 12(E59A/F60A) | | | | | |
| IL- | | 160 | Fur texture | None | 2100 pg/ml |
| 2(R38D/K43E/E61R/Q126T)/PD- | | | (moderate) | | |
| L1(mut2)-Fc/mIL-12(E59A/F60A) | | | | | |

### Example 27: Generation of IL-12/IFN-γ/PD-L1-Fc Immunomodulatory Molecules with IL-12 and IFN-γ Biological Activity Directed to PD-1-Positive Cells

Certain cytokines have synergistic action, such as IL-12 and IFN-γ. To investigate whether immunomodulatory molecules can be constructed with synergistic IL-12 and IFN-γ activity while retaining PD-1 binding dependent cytokine activity, different configurations of immunomodulatory molecules were constructed using heterodimeric PD-L1-Fc or heterodimeric PD-L2-Fc as the parental PD-1 binding protein.

### Construction of IL-12/IFN-γ/PD-L1-Fc Immunomodulatory Molecules

Heterodimeric PD-L1(mut2)-Fc and PD-L1(mut7)-Fc immunomodulatory molecules were used as the parental PD-1 binding protein (constructed in Examples 23 and 24). Heterodimeric PD-L1(mut2)-Fc has a first polypeptide chain comprising SEQ ID NO: 132, and a second polypeptide chain comprising SEQ ID NO: 134. Heterodimeric PD-L1(mut7)-Fc has a first polypeptide chain comprising SEQ ID NO: 133, and a second polypeptide chain comprising SEQ ID NO: 135.

To construct IL-12/IFN-γ/PD-L1-Fc immunomodulatory molecules, one polypeptide chain comprises no IL-12 (as control; SEQ ID NO: 132), or a single chain IL-12(E59A/F60A) polypeptide positioned at the hinge region of PD-L1(mut)-Fc (see, e.g., SEQ ID NO: 155); the pairing polypeptide chain comprises a single chain IFN-γ(A23V/A23V) homodimer positioned at the hinge region of PD-L1(mut)-Fc (from N′ to C′: PD-L1(mut) extracellular domain-GGGSG linker (SEQ ID NO: 209)-single chain IFN-γ(A23V/A23V) homodimer (SEQ ID NO: 47 or 252)-GGGGSGGG linker (SEQ ID NO: 244)-hinge (SEQ ID NO: 87)-Fc domain subunit (SEQ ID NO: 98)).

### Construction of IL-12/IFN-γ/PD-L2-Fc Immunomodulatory Molecules

Heterodimeric PD-L2(mut2)-Fc and PD-L2(mut4)-Fc immunomodulatory molecules were used as the parental PD-1 binding protein (constructed in Example 23). Heterodimeric PD-L2(mut2)-Fc has a first polypeptide chain comprising SEQ ID NO: 116, and a second polypeptide chain comprising SEQ ID NO: 118. Heterodimeric PD-L2(mut4)-Fc has a first polypeptide chain comprising SEQ ID NO: 117, and a second polypeptide chain comprising SEQ ID NO: 119.

To construct IL-12/IFN-γ/PD-L2-Fc immunomodulatory molecules, one polypeptide chain comprises from N′ to C′: PD-L2(mut) extracellular domain (e.g., SEQ ID NO: 108 or 110)-GGGGSGGG linker (SEQ ID NO: 244)-single chain IL-12(E59A/F60A) variant (e.g., SEQ ID NO: 68 or 254)-GGGGSGGG linker (SEQ ID NO: 244)-hinge (SEQ ID NO: 88)-Fc domain subunit (SEQ ID NO: 97); and one pairing polypeptide chain comprises from N′ to C′: PD-L2(mut) extracellular domain (e.g., SEQ ID NO: 108 or 110)-GGGSG linker (SEQ ID NO: 209)-single chain IFN-r(A23V/A23V) homodimer variant (SEQ ID NO: 47 or 252)-GGGGSGGG linker (SEQ ID NO: 244)-hinge (SEQ ID NO: 87)-Fc domain subunit (SEQ ID NO: 98).

### IL-12 and IFN-Y Signal Transduction Assay

HEK-Blue™ IL-12 Cells and HEK-PD-1-IL-12 cells were used to assess IL-12 signal activation activity of the immunomodulatory molecules, as described in Example 1. To assess biological activity of various IFN-γ moieties within the immunomodulatory molecules, HEK-IFN-γ-PD-1 cells were generated in-house by overexpressing human PD-1 in HEK-Blue™ IFN-γ Cells. HEK-IFN-γ reporter assay and HEK-PD-1-IFN-γ reporter assay were conducted similarly as in Example 15.

As can be seen from Table 20, both IL-12/IFN-γ/PD-L1-Fc and IL-12/IFN-γ/PD-L2-Fc immunomodulatory molecules exhibited IL-12 and IFN-γ activity only in the presence of PD-1 binding. Further, IL-12 and IFN-γ activity did not seem to be strongly impacted by the type of PD-1 binding protein used, as immunomodulatory molecules comprising PD-L1 extracellular domain and PD-L2 extracellular domain performed approximately equally.

These data indicated that both IL-12 and IFN-γ moieties when positioned at hinge retained PD-1-binding dependent activity when constructed in a trispecific immunomodulatory molecule format. Further, the IL-12 and IFN-γ moieties did not have a significant negative impact on each other's activity (compare HEK-IFN-γ-PD-1 and HEK-IL-12-PD-1 columns).

TABLE 20
IL-12 and IFN-γ biological activity of IL-12/IFN-γ/PD-L1-Fc and IL-12/
IFN-γ/PD-L2-Fc immunomodulatory molecules

| | IL-12 fusion or non- | IFN-γ fusion | | HEK- | | HEK- |
|---|---|---|---|---|---|---|
| | fusion polypeptide | polypeptide | HEK- | IFN-γ- | HEK- | IL-12- |
| Construct | SEQ ID NO: | SEQ ID NO: | IFN-γ | PD-1 | IL-12 | PD-1 |
| IFN-γ | / | / | 100% | 100% | / | / |
| rIL-12 | / | / | / | / | 100% | 100% |
| IFN-γ(A23V/A23V)/ | 132 | 184 | <0.1% | 44% | <0.2% | <0.2% |
| PD-L1(mut2)-Fc | (no IL-12) | (IFN-γ at hinge) | | | | |
| IL-12(E59A/F60A)/IFN- | 155 | 184 | <0.1% | 39% | <0.2% | 32% |
| γ(A23V/A23V)/PD-L1(mut2)-Fc | (IL-12 at hinge) | (IFN-γ at hinge) | | | | |
| IL-12(E59A/F60A)/IFN- | 156 | 185 | <0.1% | 45% | <0.2% | 43% |
| γ(A23V/A23V)/PD-L1(mut7)-Fc | (IL-12 at hinge) | (IFN-γ at hinge) | | | | |
| IL-12(E59A/F60A)/IFN- | 167 | 188 | <0.1% | 35% | <0.2% | 45% |
| γ(A23V/A23V)/PD-L2(mut2)-Fc | (IL-12 at hinge) | (IFN-γ at hinge) | | | | |
| IL-12(E59A/F60A)/IFN- | 168 | 189 | <0.1% | 40% | <0.2% | 49% |
| γ(A23V/A23V)/PD-L2(mut4)-Fc | (IL-12 at hinge) | (IFN-γ at hinge) | | | | |

### Example 28: Generation of IL-2/IL-12/CD155-Fc and IL-12/IFN-γ/CD155-Fc Immunomodulatory Molecules with IL-2, IL-12, and IFN-γ Biological Activity Directed to TIGIT-Positive Cells

The TIGIT/CD155 pathway plays a similar role as PD-1/PD-L in the inhibition of T cell functions. Like PD-1, TIGIT is highly expressed in intratumoral T cells, such as exhausted T cells. To investigate whether immunomodulatory molecules can be constructed with IL-12, IL-2, and/or IFN-γ activity dependent on binding to TIGIT expressing cells (e.g., T cells), different configurations of immunomodulatory molecules were constructed using CD155 extracellular domain (SEQ ID NO: 137) as the TIGIT binding protein.

A heterodimeric CD155-Fc was used as the parental CD155-Fc protein, comprising a first polypeptide chain (SEQ ID NO: 138) from N′ to C′: CD155 extracellular domain (SEQ ID NO: 137)-GGGGSGGG linker (SEQ TD NO: 244)-hinge (SEQ ID NO: 88)-Fc domain subunit1 (SEQ ID NO: 97)); and a second polypeptide chain (SEQ ID NO: 139) from N′ to C′: CD155 extracellular domain (SEQ ID NO: 137)-GGGGSGGG linker (SEQ ID NO: 244)-hinge (SEQ ID NO: 87)-Fc domain subunit2 (SEQ ID NO: 98).

### Construction of IL-12/CD155-Fc (IL-12 Hinge) and CD155-Fc/IL-12 (IL-12 C′) Immunomodulatory Molecules

To generate IL-12/CD155-Fc and CD155-Fc/IL-12 immunomodulatory molecules, one polypeptide chain comprises i) no IL-12 fusion (as control), or ii) IL-12 positioned in the hinge region (SEQ ID NO: 190; from N′ to C′: CD155 extracellular domain (SEQ ID NO: 137)-linker (SEQ ID NO: 244)-single chain IL-12(E59A/F60A) variant (e.g., SEQ ID NO: 68 or 254)-linker (SEQ ID NO: 244)-hinge-(SEQ ID NO: 88)-Fc domain subunit1 (SEQ ID NO: 97)), or iii) IL-12 positioned at the C-terminus of one Fc domain subunit (SEQ ID NO: 191; from N′ to C′: CD155 extracellular domain (SEQ ID NO: 137)-linker (SEQ ID NO: 244)-hinge (SEQ ID NO: 88)-Fc domain subunit1 (SEQ ID NO: 97)-linker (SEQ ID NO: 244)-single chain IL-12(E59A./F60A) variant (e.g., SEQ ID NO: 68 or 254)). The pairing polypeptide chain comprises the sequence of SEQ ID NO: 139.

### Construction of IL-2/CD155-Fc Immunomodulatory Molecules

To generate IL-2/CD155-Fc immunomodulatory molecules, one polypeptide chain comprises IL-12 or a mutant variant positioned at the hinge region (from N′ to C′: CD155 extracellular domain (SEQ ID NO: 137)-GGGSG linker (SEQ ID NO: 209)-IL-2(mut) (e.g., any of SEQ ID NOs: 26-30)-GGGGSGGG linker (SEQ ID NO: 244)-hinge (SEQ ID NO: 87)-Fc domain subunit2 (SEQ ID NO: 98)). Hence, the polypeptide chain with IL-2 moiety positioned at hinge can comprise the sequence of any of SEQ ID NOs: 247-250. The pairing polypeptide chain without IL-2 fusion comprises the sequence of SEQ ID NO: 138.

### Construction of IFN-γ/CD155-Fc Immunomodulatory Molecules

To generate IFN-γ/CD155-Fc immunomodulatory molecules, one polypeptide chain comprises i) no IFN-γ (as control; SEQ ID NO: 139), or ii) single-chain homodimer IFN-γ(A23V/A23V) positioned in the hinge region (from N′ to C′: CD155 extracellular domain (SEQ ID NO: 137)-linker (SEQ ID NO: 244)-single chain IFN-γ(A23V/A23V) homodimer variant (SEQ ID NO: 47 or 252)-linker (SEQ ID NO: 244)-hinge (SEQ ID NO: 87)-Fc domain subunit2 (SEQ ID NO: 98)). Hence, the polypeptide chain with IFN-γ moiety positioned at hinge can comprise the sequence of SEQ ID NO: 193. The pairing polypeptide chain without IFN-γ fusion comprises the sequence of SEQ ID NO: 138.

### Construction of IL-12/IL-2/CD155-Fc (IL-12 Hinge) and IL-2/CD155-Fc/IL-12 (IL-12 at C′) Immunomodulatory Molecules

IL-2/CD155-Fc (IL-2 at hinge) heterodimeric immunomodulatory molecules constructed above can be used as parental construct for making IL-12′IL-2/CD155-Fc (IL-12 at hinge) or IL-2/CD155-Fc/IL-12 (IL-12 at C′ of one of Fc subunits) immunomodulatory molecules. The polypeptide chain with IL-2 moiety positioned at hinge can comprise the sequence of any of SEQ ID NOs: 247-250. The paring polypeptide with single-chain IL-12(E59A/F60A) variant positioned at the hinge region can comprise the sequence of 190; or The paring polypeptide with single-chain IL-12(E59A/F60A) variant positioned at the C′ of the Fc subunit can comprise the sequence of 191 (see above).

### Construction of IL-12/IFN-γ/CD155-Fc (IL-12 Hinge) and IFN-Y/CD155-Fc/IL-12 (IL-12 at C′) Immunomodulatory Molecules

IFN-γ/CD155-Fc (IFN-γ at hinge) heterodimeric immunomodulatory molecules constructed above can be used as parental construct for making IL-12/IFN-γ/CD155-Fc (IL-12 at hinge) or IFN-γ/CD155-Fc/IL-12 (IL-12 at C′ of one of Fc subunits) immunomodulatory molecules. The polypeptide chain with single-chain IFN-γ(A23V/A23V) homodimer positioned at hinge can comprise the sequence of SEQ ID NO: 193. The paring polypeptide with single-chain IL-12(E59A/F60A) variant positioned at the hinge region can comprise the sequence of 190; or The paring polypeptide with single-chain IL-12(E59A/F60A) variant positioned at the C′ of the Fc subunit can comprise the sequence of 191 (see above).

### IL-12. IL-2, and IFN-γ Signal Transduction Assay

To assess biological activity of the IL-12 moieties within IL-12 containing immunomodulatory molecules, HEK-Blue™ IL-12-TIGIT cells were generated in-house by overexpressing TIGIT in HEK-Blue™ IL-12 Cells (see Example 1). To assess biological activity of the IL-2 moieties within the IL-2 containing immunomodulatory molecules, HEK-Blue™ IL-2-TIGIT cells were generated in-house by overexpressing TIGIT in HEK-Blue™ IL-2 Cells (see Example 12). To assess biological activity of various IFN-γ moieties within the IFN-γ containing immunomodulatory molecules, HEK-IFN-γ-TIGIT cells were generated in-house by overexpressing human TIGIT in HEK-Blue™ IFN-γ Cells (see Example 15).

As can be seen from Table 21, bi- and trispecific immunomodulatory molecules comprising IL-12 moiety positioned at binge showed minimal IL-12 activity without CD155/TIGIT binding; in the presence of TIGIT binding, the activity of IL-12 was rescued. Similarly, bi- and trispecific immunomodulatory molecules comprising IL-2 or IFN-γ moiety positioned at hinge region showed minimal IL-2 or IFN-γ activity without CD155/TIGIT binding; in the presence of TIGIT binding, the activity of ˜2 or IFN-γ was rescued. These data indicate that IL-12, IL-2, and IFN-γ when positioned at hinge region all retain TIGIT-binding dependent activity when constructed as bi- or tri-specific immunomodulatory molecules. Further, these data indicate that SE-12, Q-2, and IFN-γ moieties do not have significant negative impact on each other's activity when constructed as bi- or tri-specific immunomodulatory molecules.

TABLE 21
IL-2, IL-12, and IFN-γ biological activity of immunomodulatory molecules
comprising IL-2, IL-12, and/or IFN-γ directed to TIGIT-positive cells

| | IL-12 | IL-2 | IFN-γ | | | | | | |
|---|---|---|---|---|---|---|---|---|---|
| | fusion or | fusion | fusion | | | | | | |
| | non- | poly- | or non- | | | | | | |
| | fusion | peptide | fusion | | | | | | |
| | polypep- | SEQ | polypep- | | HEK- | | HEK- | | HEK- |
| | tide SEQ | ID | tide SEQ | HEK- | IL-2- | HEK- | IFN-γ- | HEK- | IL-12- |
| Construct | ID NO: | NO: | ID NO: | IL-2 | TIGIT | IFN-γ | TIGIT | IL-12 | TIGIT |
| IL-2 | / | / | / | 100% | 100% | / | / | / | / |
| IFN-γ | / | / | / | / | / | 100% | 100% | / | / |
| rIL-12 | / | / | / | / | / | / | / | 100% | 100% |
| IL- | 190 | / | 139 | <0.1% | <0.1% | n/a | n/a | <0.2% | 21% |
| 12(E59A/F60A)/ | (IL-12 | | (no IFN- | | | | | | |
| CD155-Fc | hinge) | | γ) | | | | | | |
| IL-12(E59A/F60A)/IL- | | 247 | / | 12% | 78% | n/a | n/a | <0.2% | 21% |
| 2(L18R/Q22E/R38D/ | | (IL-2 | | | | | | | |
| K43E/E61R)/CD155- | | hinge) | | | | | | | |
| Fc | | | | | | | | | |
| IL-12(E59A/F60A)/IL- | | 248 | / | 4% | 35% | n/a | n/a | <0.2% | 17% |
| 2(R38D/K43E/E61R/ | | (IL-2 | | | | | | | |
| Q126T)/CD155-Fc | | hinge) | | | | | | | |
| IL-12(E59A/F60A)/IL- | | 249 | / | 2% | 20% | n/a | n/a | <0.2% | 19% |
| 2(L18R/Q22E/R38D/ | | (IL-2 | | | | | | | |
| K43E/E61R/Q126T)/ | | hinge) | | | | | | | |
| CD155-Fc | | | | | | | | | |
| IL-12(E59A/F60A)/IL- | | 250 | / | <0.1% | <0.1% | n/a | n/a | <0.2% | 15% |
| 2(L18R/Q22E/R38D/ | | (IL-2 | | | | | | | |
| K43E/E6IRQ126T/ | | hinge) | | | | | | | |
| S130R)/CD155-Fc | | | | | | | | | |
| IL- | | / | 193 | n/a | n/a | <0.2% | 49 | <0.2% | 40% |
| 12(E59A/F60A)/IFN- | | | (IFN-γ | | | | | | |
| γ(A23V/A23V)/ | | | hinge) | | | | | | |
| CD155-Fc | | | | | | | | | |
| IL-12(E59A/F60A)/ | 191 | / | 139 | <0.1% | <0.1% | n/a | n/a | 2% | 35% |
| CD155-Fc | (IL-12 at | | (no IFN- | | | | | | |
| | C′) | | γ) | | | | | | |
| IL-2(L18R/Q22E/ | | 247 | / | 15% | 81% | n/a | n/a | 1% | 42% |
| R38D/K43E/E61R)/ | | (IL-2 | | | | | | | |
| CD155-Fc/IL- | | hinge) | | | | | | | |
| 12(E59A/F60A) | | | | | | | | | |
| IL-2(R38D/K43E/ | | 248 | / | 3% | 28% | n/a | n/a | 2% | 32% |
| E61R/Q126T)/CD155- | | (IL-2 | | | | | | | |
| Fc/IL-12(E59A/F60A) | | hinge) | | | | | | | |
| IL-2(L18R/Q22E/ | | 249 | / | 5% | 18% | n/a | n/a | <0.2% | 29% |
| R38D/K43E/E61R/ | | (IL-2 | | | | | | | |
| Q126T)/CD155- | | hinge) | | | | | | | |
| Fc/IL-12(E59A/F60A) | | | | | | | | | |
| IL-2(L18R/Q22E/ | | 250 | / | <0.1% | <0.1% | n/a | n/a | 3% | 24% |
| R38D/K43E/E61R/ | | (IL-2 | | | | | | | |
| Q126T/S130R)/ | | hinge) | | | | | | | |
| CD155-Fc/IL- | | | | | | | | | |
| 12(E59A/F60A) | | | | | | | | | |
| IFN-γ(A23V/ | | / | 193 | n/a | n/a | <0.2% | 45% | 2% | 60% |
| A23V)/CD155-Fc/ | | | (IFN-γ | | | | | | |
| IL-12(E59A/F60A) | | | hinge) | | | | | | |
| CD155-Fc | 138 | / | 139 | <0.1% | <0.1% | n/a | n/a | n/a | n/a |
| (control) | (no IL-12) | | (no IFN- | | | | | | |
| | | | γ | | | | | | |
| IL-2(L18R/Q22E/ | | 247 | / | 9% | 56% | n/a | n/a | n/a | n/a |
| R38D/K43E/E61R)/ | | (IL-2 | | | | | | | |
| CD155-Fc | | hinge) | | | | | | | |
| IL-2(R38D/K43E/ | | 248 | / | 3% | 29% | n/a | n/a | n/a | n/a |
| E61R/Q126T)/ | | (IL-2 | | | | | | | |
| CD155-Fc | | hinge) | | | | | | | |
| IL-2(L18R/Q22E/ | | 249 | / | 2% | 15% | n/a | n/a | n/a | n/a |
| R38D/K43E/E61R/ | | (IL-2 | | | | | | | |
| Q126T)/CD155-Fc | | hinge) | | | | | | | |
| IL- | | 250 | / | <0.1% | <0.1% | n/a | n/a | n/a | n/a |
| 2(L18R/Q22E/R38D/ | | (IL-2 | | | | | | | |
| K43E/E61R/Q126T/ | | hinge) | | | | | | | |
| S130R)/CD 155-Fc | | | | | | | | | |
| IN-γ(A23V/A23V)/ | | / | 193 | n/a | n/a | <0.2% | 35% | n/a | n/a |
| CD155-Fc | | | (IFN-γ | | | | | | |
| | | | hinge) | | | | | | |

SEQUENCE LISTING
SEQ ID NO: 1 (wildtype human CTLA-4 extracellular domain-hinge-IgG1 Fc mutant2; CTLA-4
extracellular domain is underlined; hinge is bolded; linker is bolded and underlined)
KAMHVAQPAVVLASSRGIASFVCEYASPGKATEVRVTVLRQADSQVTEVCAATYMMGNELTFLDDSICTG
TSSGNQVNLTIQGLRAMDTGLYICKVELMYPPPYYLGIGNGTQIYVIDPEPCPDSDGSGDKTHTCPPCPAP

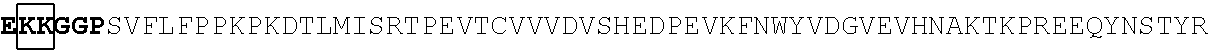

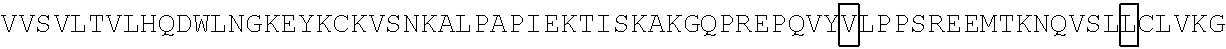

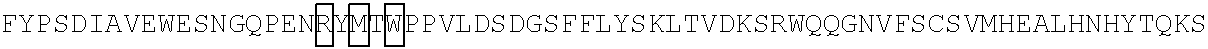

LSLSPGK
SEQ ID NO: 2 (wildtype human CTLA-4 extracellular domain-linker-hinge-IgG1 Fc mutant1-linker-single-
chain IL-12 mutant heterodimer IL-12B (p40 E59A/F60A)-linker-IL-12A (wt p35); CTLA-4 extracellular
domain is underlined; linker is bolded and underlined; hinge is bolded; IL-12 subunits are italicized)
KAMHVAQPAVVLASSRGIASFVCEYASPGKATEVRVTVLRQADSQVTEVCAATYMMGNELTFLDDSICTG
TSSGNQVNLTIQGLRAMDTGLYICKVELMYPPPYYLGIGNGTQIYVIDPEPCPDSDGSGDKTHTCPPCPAP

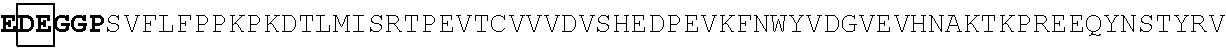

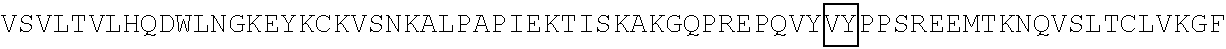

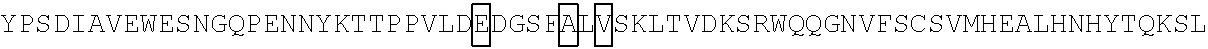

SLSPGKGGGGGGGGSGGGGSIWELKKDVYVVELDWYPDAPGEMVVLTCDTPEEDGITWTLDOSSEVLGSGK

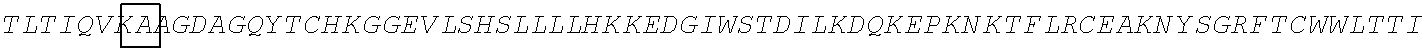

STDLTFSVKSSRGSSDPQGVTCGAATLSAERVRGDNKEYEYSVECQEDSACPAAEESLPIEVMVDAVHKLKYENYT
SSFFIRDIIKPDPPKNLQLKPLKNSRQVEVSWEYPDTWSTPHSYFSLTFCVQVQGKSKREKKDRVFTDKTSATVICR
KNASISVRAQDRYYSSSWSEWASVPCSGGGGSGGGGSGGGGSGGGGSGRNLPVATPDPGMFPCLHHSQNLLR
AVSNMLQKARQTLEFYPCTSEEIDHEDITKDKTSTVEACLPLELTKNESCLNSRETSFITNGSCLASRKTSFMMALC
LSSIYEDLKMYQVEFKTMNAKLLMDPKRQIFLDQNMLAVIDELMQALNENSETVPQKSSLEEPDFYKTKIKLCILL
HAFRIRAVTIDRVMSYLNAS
SEQ ID NO: 3 (wildtype human CTLA-4 extracellular domain-linker-hinge-IgG1 Fc mutant1-linker-single-
chain IL-12 mutant heterodimer IL-12B (p40 F60A)-linker-IL-12A (wt p35) ; CTLA-4 extracellular domain
is underlined; linker is bolded and underlined; hinge is bolded; IL-12 subunits are italicized)
KAMHVAQPAVVLASSRGIASFVCEYASPGKATEVRVTVLROADSQVTEVCAATYMMGNELTFLDDSICTG
TSSGNQVNLTIQGLRAMDTGLYICKVELMYPPPYYLGIGNGTQIYVIDPEPCPDSDGSGDKTHTCPPCPAP

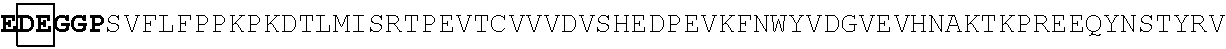

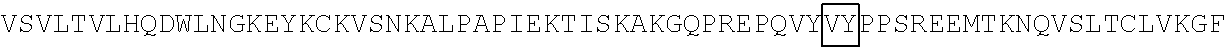

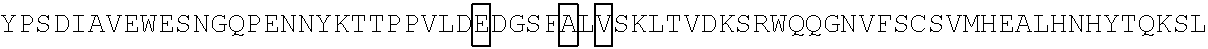

SLSPGKGGGGGGGGGGGGSIWELKKDVYVVELDWYPDAPGEMVVLTCDTPEEDGITWTLDQSSEVLGSGK

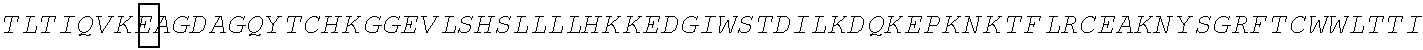

STDLTFSVKSSRGSSDPQGVTCGAATLSAERVRGDNKEYEYSVECQEDSACPAAEESLPIEVMVDAVHKLKYENYT
SSFFIRDIIKPDPPKNLQLKPLKNSROVEVSWEYPDTWSTPHSYFSLTFCVQVQGKSKREKKDRVFTDKTSATVICR
KNASISVRAQDRYYSSSWSEWASVPCSGGGGSGGGGSGGGGSGGGGSGRNLPVATPDPGMFPCLHHSQNLLR
AVSNMLQKARQTLEFYPCTSEEIDHEDITKDKTSTVEACLPLELTKNESCLNSRETSFIINGSCLASRKTSFMMALC
LSSIYEDLKMYOVEFKTMNAKLLMDPKRQIFLDQNMLAVIDELMQALNENSETVPQKSSLEEPDFYKTKIKLCILL
HAFRIRAVTIDRVMSYLNAS
SEQ ID NO: 4 (wildtype human CTLA-4 extracellular domain-linker-single-chain IL-12 mutant heterodimer
IL-12B (p40 E59A/F60A)-linker-IL-12A (wt p35)-hinge-IgG1 Fc mutant1; CTLA-4 extracellular domain is
underlined; linker is bolded and underlined; hinge is bolded; IL-12 subunits are italicized)
KAMHVAQPAVVLASSRGIASFVCEYASPGKATEVRVTVLRQADSQVTEVCAATYMMGNELTFLDDSICTG
TSSGNQVNLTIQGLRAMDTGLYICKVELMYPPPYYLGIGNGTQIYVIDPEPCPDSDGSGIWELKKDVYVVELD

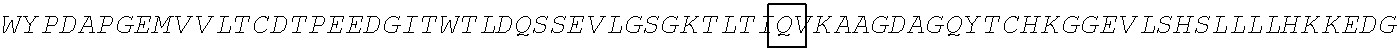

IWSTDILKDOKEPKNKTFLRCEAKNYSGRFTCWWLTTISTDLTFSVKSSRGSSDPQGVTCGAATLSAERVRGDNKE
YEYSVECQEDSACPAAEESLPIEVMVDAVHKLKYENYTSSFFIRDIIKPDPPKNLQLKPLKNSROVEVSWEYPDTWS
TPHSYFSLTFCVQVQGKSKREKKDRVFTDKTSATVICRKNASISVRAODRYYSSSWSEWASVPCSGGGGSGGGGS
GGGGSGGGGSGRNLPVATPDPGMFPCLHHSQNLLRAVSNMLQKARQTLEFYPCTSEEIDHEDITKDKTSTVEA
CLPLELTKNESCLNSRETSFITNGSCLASRKTSFMMALCLSSIYEDLKMYQVEFKTMNAKLLMDPKRQIFLDQNML

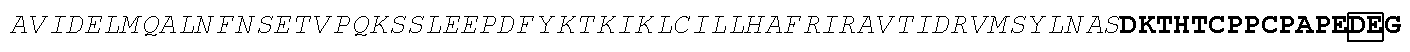

GPSVFLFPPKPKDTLMISRTPEVTCVVVDVSHEDPEVKFNWYVDGVEVHNAKTKPREEQYNSTYRVVSVL

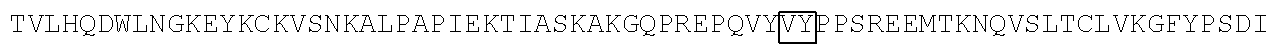

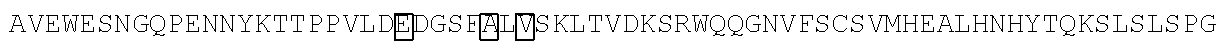

K
SEQ ID NO: 5 (wildtype human CTLA-4 extracellular domain-linker-single-chain IL-12 mutant heterodimer
IL-12B (p40 F60A)-linker-IL-12A (wt p35)-hinge-IgG1 Fe mutant1; CTLA-4 extracellular domain is
underlined; linker is bolded and underlined; hinge is bolded; IL-12 subunits are italicized)
KAMHVAQPAVVLASSRGIASFVCEYASPGKATEVRVTVLRQADSQVTEVCAATYMMGNELTELDDSICTG
TSSGNOVNLTIQGLRAMDTGLYICKVELMYPPPYYLGIGNGTQIYVIDPEPCPDSDGSGIWELKKDVYVVELD

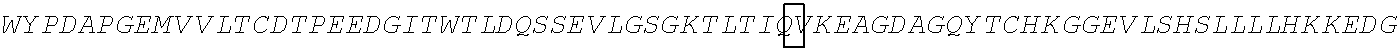

IWSTDILKDOKEPKNKTFLRCEAKNYSGRFTCWWLTTISTDLTFSVKSSRGSSDPQGVTCGAATLSAERVRGDNKE
YEYSVECOEDSACPAAEESLPIEVMVDAVHKLKYENYTSSFFIRDIIKPDPPKNLQLKPLKNSROVEVSWEYPDTWS
TPHSYFSLTFCVQVQGKSKREKKDRVFTDKTSATVICRKNASISVRAQDRYYSSSWSEWASVPCSGGGGSGGGGS
GGGGSGGGGSGRNLPVATPDPGMFPCLHHSONLLRAVSNMLQKARQTLEFYPCTSEEIDHEDITKDKTSTVEA
CLPLELTKNESCLNSRETSFITNGSCLASRKTSFMMALCLSSIYEDLKMYQVEFKTMNAKLLMDPKRQIFLDQNML

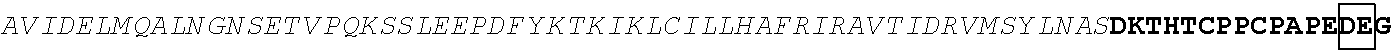

GPSVFLFPPKPKDTLMISRTPEVTCVVVDVSHEDPEVKFNWYVDGVEVHNAKTKPREEQYNSTYRVVSVL

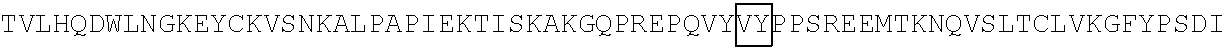

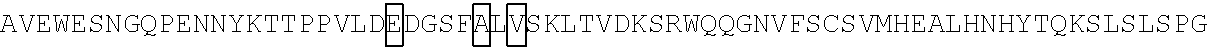

K
SEQ ID NO: 6 (wildtype human PD-L1 WT extracellular domain-hinge-IgG1 Fc mutant2; PD-L1
extracellular domain is underlined; hinge is bolded; linker is bolded and underlined)
FTVTVPKDLYVVEYGSNMTIECKFPVEKQLDLAALIVYWEMEDKNIIQFVHGEEDLKVOHSSYRORARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFYCTERRLDPEENHTAELVIPELPL

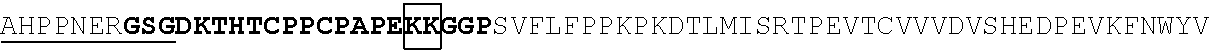

DGVEVHNAKTKPREEQYNSTYRVVSVLTVLHQDWLNGKEYKCKVSNKALPAPIEKTISKAKGQPREPQVY

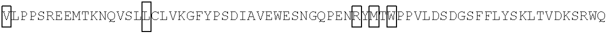

QGNVFSCSVMHEALHNHYTQKSLSLSPGK
SEQ ID NO: 7 (wildtype human PD-L1 WT extracellular domain-linker-hinge-IgG1 Fc mutant1-linker-
single-chain IL-12 mutant heterodimer IL-12B (p40 E59A/F60A)-linker-IL-12A (wt p35); PD-L1
extracellular domain is underlined; hinge is bolded; linker is bolded and underlined)
FTVTVPKDLYVVEYGSNMTIECKFPVEKQLDLAALIVYWEMEDKNIIQFVHGEEDLKVQHSSYRORARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFYCTFRRLDPEENHTAELVIPELPL

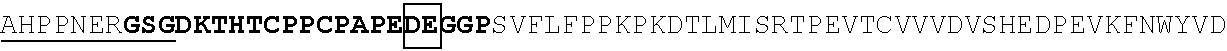

GVEVHNAKTKPREEQYNSTYRVVSVLTVLHQDWLNGKEYKCKVSNKALPAPIEKTISKAKGQPREPQVY

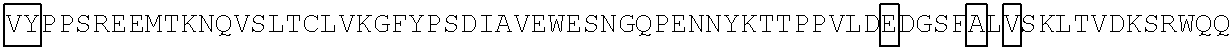

GNVFSCSVMHEALHNHYTQKSLSLSPGKGGGGSGGGGSGGGGSIWELKKDVYVVELDWYPDAPGEMVVLT

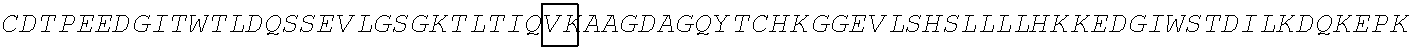

NKTFLRCEAKNYSGRFTCWWLTTISTDLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSRQVEVSWEYPDTWSTPHSYFSLTFCVQV
QGKSKREKKDRVFTDKTSATVICRKNASISVRAQDRYYSSSWSEWASVPCSGGGGSGGGGSGGGGSGGGGSG
RNLPVATPDPGMFPCLHHSONLLRAVSNMLQKARQTLEFYPCTSEEIDHEDITKDKTSTVEACLPLELTKNESCLN
SRETSFITNGSCLASRKTSFMMALCLSSIYEDLKMYQVEFKIMNAKLLMDPKRQIFLDQNMLAVIDELMOALNENS
ETVPQKSSLEEPDFYKTKIKLCILLHAFRIRAVTIDRVMSYLNAS
SEQ ID NO: 8 (wildtype human PD-L1 WT extracellular domain-linker-hinge-IgG1 Fc mutant1-linker-
single-chain IL-12 mutant heterodimer IL-12B (p40 F60A)-linker-IL-12A (wt p35); PD-L1 extracellular
domain is underlined; hinge is bolded; linker is bolded and underlined)
FTVTVPKDLYVVEYGSNMTIECKFPVEKQLDLAALIVYWEMEDKNIIQFVHGEEDLKVQHSSYRQRARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFYCTFRRLDPEENHTAELVIPELPL

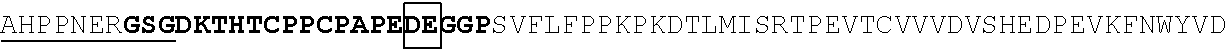

GVEVHNAKTKPREEQYNSTYRVVSVLTVLHQDWLNGKEYKCKVSNKALPAPIEKTISKAKGQPREPQVY

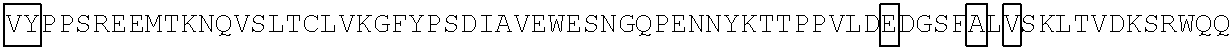

GNVFSCSVMHEALHNHYTOKSLSLSPGKGGGGSGGGGSGGGGSIWELKKDVYVVELDWYPDAPGEMVVLT

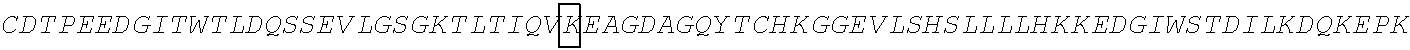

NKTFLRCEAKNYSGRFTCWWLTTISTDLTFSVKSSRGSSDPQGVTCGAATLSAERVRGDNKEYEYSVECOEDSACP
AAEESLPIEVMVDAVHKLKYENYTSSFFIRDIKPDPPKNLQLKPLKNSRQVEVSWEYPDTWSTPHSYFSLTFCVQV
QGKSKREKKDRVFTDKTSATVICRKNASISVRAQDRYYSSSWSEWASVPCSGGGGSGGGGSGGGGSGGGGSG
RNLPVATPDPGMFPCLHHSQNLLRAVSNMLQKARQTLEFYPCTSEEIDHEDITKDKTSTVEACLPLELTKNESCLN
SRETSFITNGSCLASRKTSFMMALCLSSIYEDLKMYQVEFKTMNAKLLMDPKRQIFLDQNMLAVIDELMQALNENS
ETVPQKSSLEEPDFYKTKIKLCILLHAFRIRAVTIDRVMSYLNAS
SEQ ID NO: 9 (wildtype human PD-L1 WT extracellular domain-linker-single-chain IL-12 mutant
heterodimer IL~12B (p40 E59A/F60A)-linker-IL-12A (wt p35)-hinge-IgG1 Fc mutant1; PD-L1 extracellular
domain is underlined; linker is bolded and underlined; hinge is bolded; IL-12 subunits are italicized)
FTVTVPKDLYVVEYGSNMTIECKFPVEKQLDLAALIVYWEMEDKNIIQFVHGEEDLKVQHSSYRORARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFYCTFRRLDPEENHTAELVIPELPL

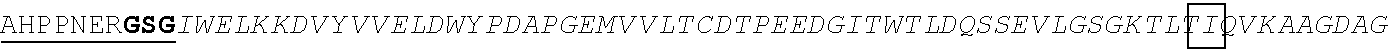

QYTCHKGGEVLSHSLLLLHKKEDGIWSTDILKDQKEPKNKTFLRCEAKNYSGRFTCWWLTTISTDLTESVKSSRGS
SDPQGVTCGAATLSAERVRGDNKEYEYSVECQEDSACPAAEESLPIEVMVDAVHKLKYENYTSSFFIRDIIKPDPPK
NLQLKPLKNSRQVEVSWEYPDTWSTPHSYFSLTFCVQVQGKSKREKKDRVFTDKTSATVICRKNASISVRAQDRYY
SSSWSEWASVPCSGGGGSGGGGSGGGGSGGGGSGRNLPVATPDPGMFPCLHHSQNLLRAVSNMLQKARQTL
EFYPCTSEEIDHEDITKDKTSTVEACLPLELTKNESCLNSRETSFITNGSCLASRKTSFMMALCLSSIYEDLKMYOVE
FKTMNAKLLMDPKROIFLDQNMLAVIDELMQALNENSETVPQKSSLEEPDFYKTKIKLCILLHAFRIRAVTIDRVM

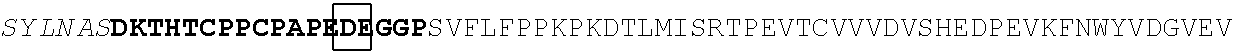

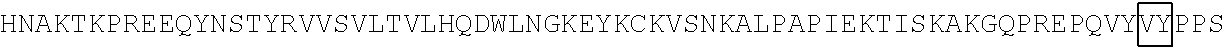

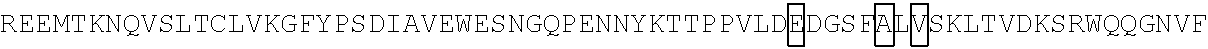

SCSVMHEALHNHYTQKSLSLSPGK
SEQ ID NO: 10 (wildtype human PD-L1 WT extracellular domain-linker-single-chain IL-12 mutant
heterodimer IL-12B (p40 F60A)-linker-IL-12A (wt p35)-hinge-IgG1 Fc mutant1; PD-L1 extracellular domain
is underlined; linker is bolded and underlined; hinge is bolded; IL-12 subunits are italicized)
FTVTVPKDLYVVEYGSNMTIECKFPVEKQLDLAALIVYWEMEDKNIIQFVHGEEDLKVOHSSYRORARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLENVISTLRINTTTNEIFYCTFRRLDPEENHTAELVIPELPL

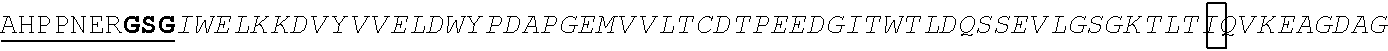

QYTCHKGGEVLSHSLLLLHKKEDGIWSTDILKDQKEPKNKTFLRCEAKNYSGRFTCWWLTTISTDLTFSVKSSRGS
SDPQGVTCGAATLSAERVRGDNKEYEYSVECQEDSACPAAEESLPIEVMVDAVHKLKYENYTSSFFIRDIIKPDPPK
NLQLKPLKNSRQVEVSWEYPDTWSTPHSYFSLTFCVQVQGKSKREKKDRVFTDKTSATVICRKNASISVRAODRYY
SSSWSEWASVPCSGGGGSGGGGSGGGGSGGGGSGRNLPVATPDPGMFPCLHHSQNLLRAVSNMLOKARQTL
EFYPCTSEEIDHEDITKDKTSTVEACLPLELTKNESCLNSRETSFITNGSCLASRKTSFMMALCLSSIYEDLKMYOVE
FKTMNAKLLMDPKRQIFLDQNMLAVIDELMQALNENSETVPQKSSLEEPDFYKTKIKLCILLHAFRIRAVTIDRVM

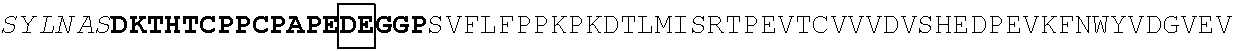

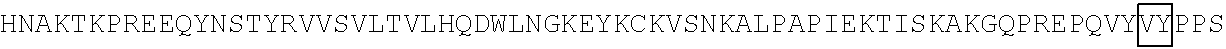

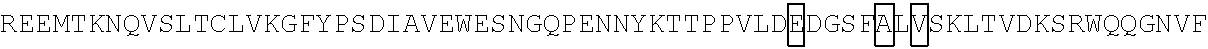

SCSVMHEALHNHYTQKSLSLSPGK.
SEQ ID NO: 11 (human PD-L1 mutant extracellular domain-hinge-IgG1 Fc mutant2; PD-L1 extracellular
domain is underlined; hinge is bolded; linker is bolded and underlined)

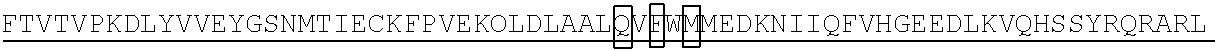

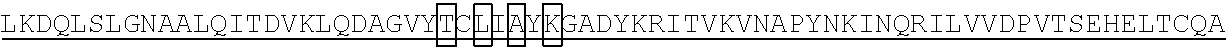

EGYPKAEVIWTSSDHQVLSGKTTTTNSKREEKLENVTSTLRINTTTNEIFYCTERRLDPEENHTAELVIPELPL

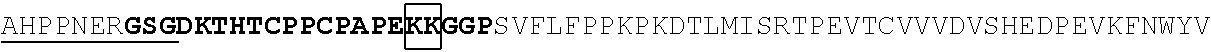

DGVEVHNAKTKPREEQYNSTYRVVSVLTVLHQDWLNGKEYKCKVSNKALPAPIEKTISKAKGQPREPQVY

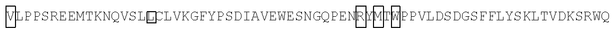

QGNVFSCSVMHEALHNHYTQKSLSLSPGK
SEQ ID NO: 12 (human PD-L1 mutant extracellular domain-linker-binge-IgG1 Fc mutant1-linker-single-
chain IL-12 mutant heterodimer IL-12B (p40 E59A/F60A)-linker-IL-12A (wt p35); PD-L1 extracellular
domain is underlined; linker is bolded and underlined; hinge is bolded; IL-12 subunits are italicized)

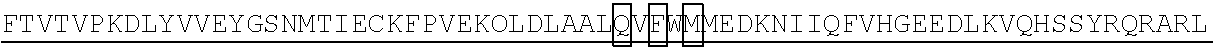

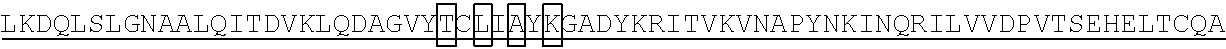

EGYPKAEVIWTSSDHQVLSGKTTTTNSKREEKLFNVTSTLRINTTTNEIFYCTFRRLDPEENHTAELVIPELPL

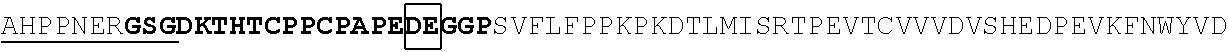

GVEVHNAKTKPREEQYNSTYRVVSVLTVLHQDWLNGKEYKCKVSNKALPAPIEKTISKAK GQPREPQVY

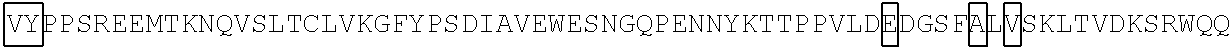

GNVFSCSVMHEALHNHYTQKSLSLSPGKGGGGSGGGGSGGGGSIWELKKDVYVVELDWYPDAPGEMVVLT

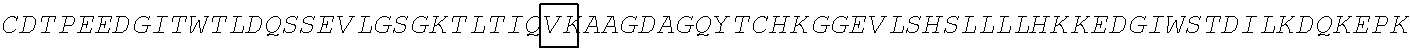

NKTFLRCEAKNYSGRFTCWWLTTISTDLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSROVEVSWEYPDTWSTPHSYFSLTFCVQV
QGKSKREKKDRVFTDKTSATVICRKNASISVRAQDRYYSSSWSEWASVPCSGGGGSGGGGSGGGGSGGGGSG
RNLPVATPDPGMFPCLHHSQNLLRAVSNMLQKARQTLEFYPCTSEEIDHEDITKDKTSTVEACLPLELTKNESCLN
SRETSFITNGSCLASRKTSFMMALCLSSIYEDLKMYQVEFKTMNAKLLMDPKRQIFLDQNMLAVIDELMQALNENS
ETVPQKSSLEEPDFYKTKIKLCILLHAFRIRAVTIDRVMSYLNAS
SEQ ID NO: 13 (human PD-L1 mutant extracellular domain-linker-hinge-IgG1 Fc mutant1-linker-single-
chain IL-12 mutant heterodimer IL-12B (p40 F60A)-linker-IL-12A (wt p35); PD-L1 extracellular domain is
underlined; linker is bolded and underlined; hinge is bolded; IL-12 subunits are italicized)

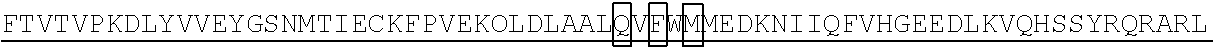

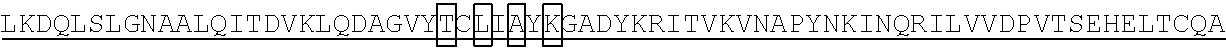

EGYPKAEVIWTSSDHQVLSGKTTTTNSKREEKLENVTSTLRINTTTNEIFYCTFRRLDPEENHTAELVIPELPL

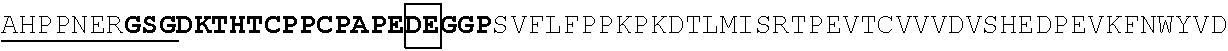

GVEVHNAKTKPREEQYNSTYRVVSVLTVLHQDWLNGKEYKCKVSNKALPAPIEKTISKAKGQPREPQVY

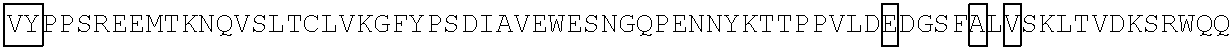

GNVFSCSVMHEALHNHYTQKSLSLSPGKGGGGSGGGGGGGGSIWELKKDVYVVELDWYPDAPGEMVVLT

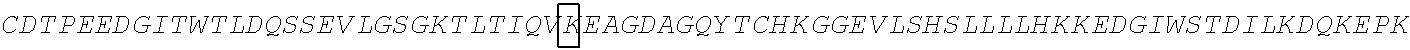

NKTFLRCEAKNYSGRFTCWWLTTISTDLTFSVKSSRGSSDPQGVTCGAATLSAERVRGDNKEYEYSVECOEDSACP
AAEESLPIEVMVDAVHKLKYENYTSSFFIRDIIKPDPPKNLQLKPLKNSRQVEVSWEYPDTWSTPHSYFSLTFCVQV
QGKSKREKKDRVFTDKTSATVICRKNASISVRAQDRYYSSSWSEWASVPCSGGGGSGGGGSGGGGSGGGGSG
RNLPVATPDPGMFPCLHHSQNLLRAVSNMLQKARQTLEFYPCTSEEIDHEDITKDKTSTVEACLPLELTKNESCLN
SRETSFITNGSCLASRKTSFMMALCLSSIYEDLKMYQVEFKTMNAKLLMDPKRQIFLDQNMLAVIDELMOALNENS
ETVPQKSSLEEPDFYKTKIKLCILLHAFRIRAVTIDRVMSYLNAS
SEQ ID NO: 14 (human PD-L1 mutant extracellular domain-linker-single-chain IL-12 mutant heterodimer
IL-12B (p40 E59A/F60A)-linker-IL-12A (wt p35)-hinge-IgG1 Fc mutant1; PD-L1 extracellular domain is
underlined; linker is bolded and underlined; hinge is bolded; IL-12 subunits are italicized)

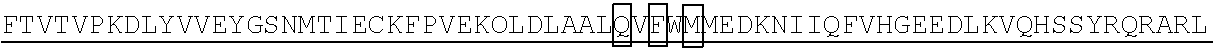

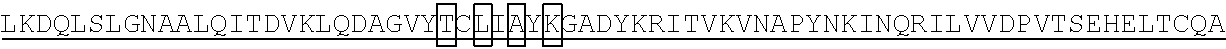

EGYPKAEVIWTSSDHQVLSGKTTTTNSKREEKLFNVTSTLRINTTTNEIFYCTFRRLDPEENHTAELVIPELPL

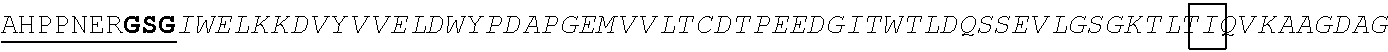

QYTCHKGGEVLSHSLLLLHKKEDGIWSTDILKDQKEPKNKTFLRCEAKNYSGRFTCWWLTTISTDLTFSVKSSRGS
SDPQGVTCGAATLSAERVRGDNKEYEYSVECQEDSACPAAEESLPIEVMVDAVHKLKYENYTSSFFIRDIIKPDPPK
NLQLKPLKNSRQVEVSWEYPDTWSTPHSYFSLTFCVQVQGKSKREKKDRVFTDKTSATVICRKNASISVRAQDRYY
SSSWSEWASVPCSGGGGSGGGGSGGGGSGGGGSGRNLPVATPDPGMFPCLHHSQNLLRAVSNMLQKARQTL
EFYPCTSEEIDHEDITKDKTSTVEACLPLELTKNESCLNSRETSFITNGSCLASRKTSFMMALCLSSIYEDLKMYQVE
FKTMNAKLLMDPKROIFLDQNMLAVIDELMQALNENSETVPQKSSLEEPDFYKTKIKLCILLHAFRIRAVTIDRVM

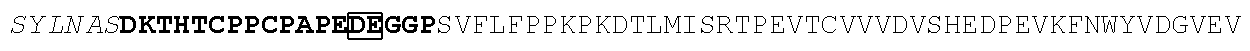

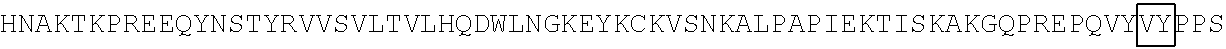

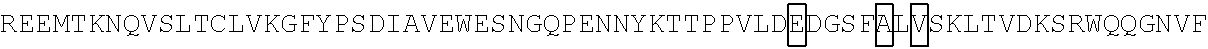

SCSVMHEALHNHYTQKSLSLSPGK
SEQ ID NO: 15 (human PD-L1 mutant extracellular domain-linker-single-chain IL-12 mutant heterodimer
IL-12B (p40 F60A)-linker-IL~12A (wt p35)-hinge-IgG1 Fc mutant1; PD-L1 extracellular domain is
underlined; linker is bolded and underlined; hinge is bolded; IL-12 subunits are italicized)

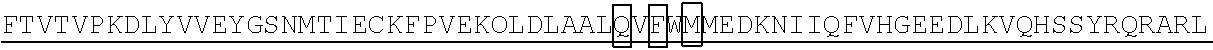

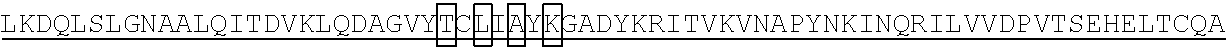

EGYPKAEVIWTSSDHQVLSGKTTTTNSKREEKLFNVTSTLRINTTTNEIFYCTFRRLDPEENHTAELVIPELPL

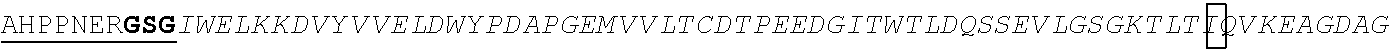

QYTCHKGGEVLSHSLLLLHKKEDGIWSTDILKDQKEPKNKTFLRCEAKNYSGRFTCWWLTTISTDLTFSVKSSRGS
SDPQGVTCGAATLSAERVRGDNKEYEYSVECQEDSACPAAEESLPIEVMVDAVHKLKYENYTSSFFIRDIIKPDPPK
NLQLKPLKNSRQVEVSWEYPDTWSTPHSYFSLTFCVQVQGKSKREKKDRVFTDKTSATVICRKNASISVRAQDRYY
SSSWSEWASVPCSGGGGSGGGGSGGGGSGGGGSGRNLPVATPDPGMFPCLHHSQNLLRAVSNMLQKARQTL
EFYPCTSEEIDHEDITKDKTSTVEACLPLELTKNESCLNSRETSFITNGSCLASRKTSEMMALCLSSIYEDLKMYQVE
FKTMNAKLLMDPKRQIFLDQNMLAVIDELMQALNENSETVPQKSSLEEPDFYKTKIKLCILLHAFRIRAVTIDRVM

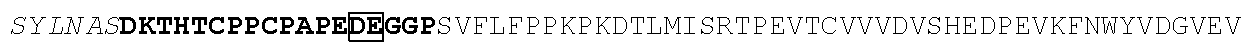

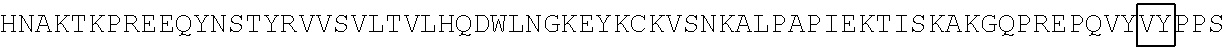

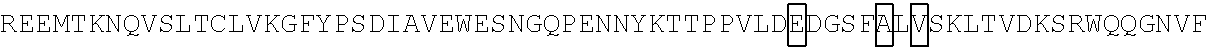

SCSVMHEALHNHYTQKSLSLSPGK
SEQ ID NO: 16 (wildtype human PD-L2 extracellular domain-hinge-IgG1 Fc mutant2; PD-L2 extracellular
domain is underlined; hinge is bolded; linker is bolded and underlined)
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRIPEGLYQVTSVLRLKPPPGRNFSCVFWNTHVRELTLASIDLQSQMEPRTHPTGSGDKTHTCPPCPA

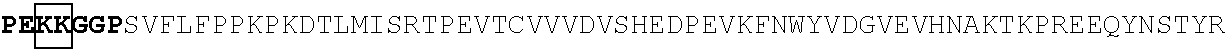

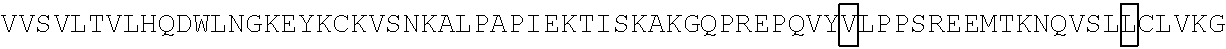

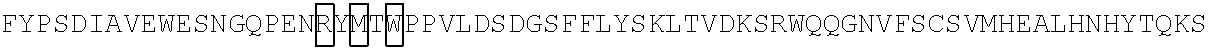

LSLSPGK
SEQ ID NO: 17 (wildtype human PD-L2 extracellular domain-linker-single-chain IL-12 mutant heterodimer
IL-12B (p40 E59A/F60A)-linker-IL~12A (wt p35)-hinge-IgG1 Fc mutant1; PD-L2 extracellular domain is
underlined; linker is bolded and underlined; hinge is bolded; IL-12 subunits are italicized)
LFTVTVPKELYIIEHGSNVTLECNEDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGIWELKKDVYVVEL

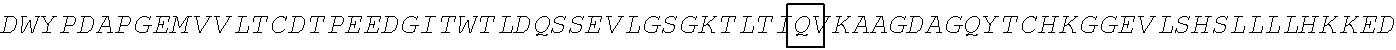

GIWSTDILKDOKEPKNKTFLRCEAKNYSGRFTCWWLTTISTDLTFSVKSSRGSSDPQGVTCGAATLSAERVRGDNK
EYEYSVECOEDSACPAAEESLPIEVMVDAVHKLKYENYTSSFFIRDIIKPDPPKNLQLKPLKNSRQVEVSWEYPDTW
STPHSYFSLIFCVQVQGKSKREKKDRVFTDKTSATVICRKNASISVRAQDRYYSSSWSEWASVPCSGGGGSGGGG
SGGGGSGGGGSGRNLPVATPDPGMFPCLHHSQNLLRAVSNMLQKARQTLEFYPCTSEEIDHEDITKDKTSTVE
ACLPLELTKNESCLNSRETSFITNGSCLASRKTSFMMALCLSSIYEDLKMYQVEFKTMNAKLLMDPKRQIFLDQNM

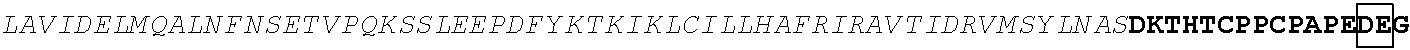

GPSVFLFPPKPKDTLMISRTPEVTCVVVDVSHEDPEVKFNWYVDGVEVHNAKTKPREEQYNSTYRVVSVL

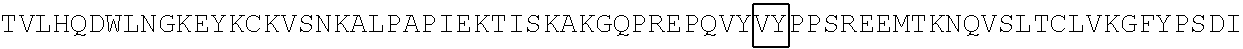

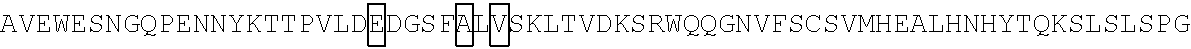

K
SEQ ID NO: 18 (wildtype human PD-L2 extracellular domain-linker-single-chain IL-12 mutant heterodimer
IL-12B (p40 F60A)-linker-IL-12A (wt p35)-hinge-IgG1 Fc mutant1; PD-L2 extracellular domain is
underlined; linker is bolded and underlined; hinge is bolded; IL-12 subanits are italicized)
LFTVTVPKELYIIEHGSNVTLECNFDTGSHVNLGAITASLQKVENDTSPHRERATLLEEQLPLGKASFHIPOV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGIWELKKDVYVVEL

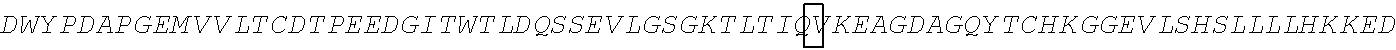

GIWSTDILKDOKEPKNKTFLRCEAKNYSGRFTCWWLTTISTDLTFSVKSSRGSSDPQGVTCGAATLSAERVRGDNK
EYEYSVECQEDSACPAAEESLPIEVMVDAVHKLKYENYTSSFFIRDIIKPDPPKNLQLKPLKNSRQVEVSWEYPDTW
STPHSYFSLTFCVQVQGKSKREKKDRVFTDKTSATVICRKNASISVRAQDRYYSSSWSEWASVPCSGGGGGGGG
SGGGGSGGGGSGRNLPVATPDPGMFPCLHHSONLLRAVSNMLQKARQTLEFYPCTSEEIDHEDITKDKTSTVE
ACLPLELTKNESCLNSRETSFITNGSCLASRKTSEMMALCLSSIYEDLKMYQVEFKTMNAKLLMDPKRQIFLDQNM

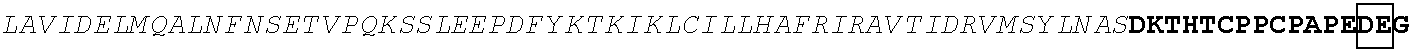

GPSVFLFPPKPKDTLMISRTPEVTCVVVDVSHEDPEVKFNWYVDGVEVHNAKTKPREEQYNSTYRVVSVL

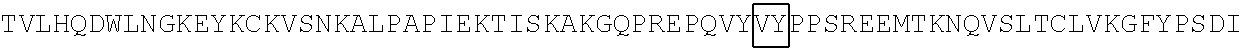

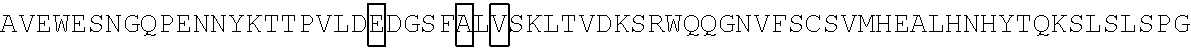

K
SEQ ID NO: 19 (wildtype human PD-L2 extracellular domain-linker-hinge-IgG1 Fc mutant1-linker-single-
chain IL-12 mutant heterodimer IL-12B (p40 E59A/F60A)-linker-IL-12A (wt p35); PD-L2 extracellular
domain is underlined; linker is bolded and underlined; hinge is bolded; IL-12 subunits are italicized)
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLILKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGDKTHTCPPCPA

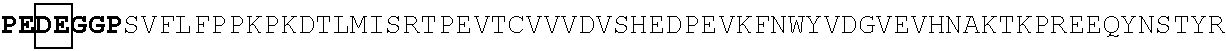

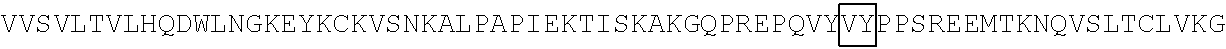

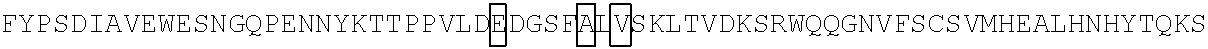

LSLSPGKGGGGSGGGGGGGGSIWELKKDVYVVELDWYPDAPGEMVVLTCDTPEEDGITWTLDQSSEVLGSG

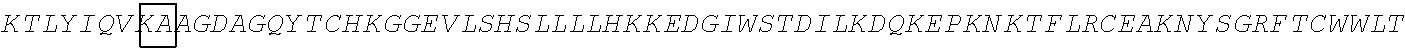

TISTDLTFSVKSSRGSSDPQGVTCGAATLSAERVRGDNKEYEYSVECQEDSACPAAEESLPIEVMVDAVHKLKYENY
TSSFFIRDIKPDPPKNLQLKPLKNSROVEVSWEYPDTWSTPHSYFSLTFCVQVQGKSKREKKDRVFTDKTSATVIC
RKNASISVRAQDRYYSSSWSEWASVPCSGGGGSGGGGSGGGGSGGGGSGRNLPVATPDPGMFPCLHHSQNLL
RAVSNMLOKARQTLEFYPCTSEEIDHEDITKDKTSTVEACLPLELTKNESCLNSRETSFITNGSCLASRKTSEMMAL
CLSSIYEDLKMYQVEFKTMNAKLLMDPKRQIFLDQNMLAVIDELMQALNENSETVPQKSSLEEPDFYKTKIKLCIL
LHAFRIRAVTIDRVMSYLNAS
SEQ ID NO: 20 (wildtype human PD-L2 extracellular domain-linker-hinge-IgG1 Fc mutant1-linker-single-
chain IL-12 mutant heterodimer IL-12B (p40 F60A)-linker-IL-12A (wt p35); PD-L2 extracellular domain is
underlined; linker is bolded and underlined; hinge is bolded; IL-12 subunits are italicized)
LFTVTVPKELYIIEHGSNVTLECNEDTGSHVNLGAITASLQKVENDTSPHRERATLLEEQLPLGKASFHIPQV
QVRDEGOYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGDKTHTCPPCPA

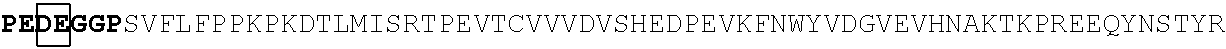

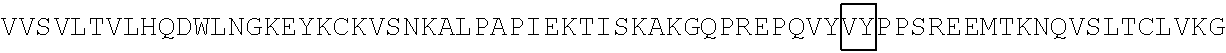

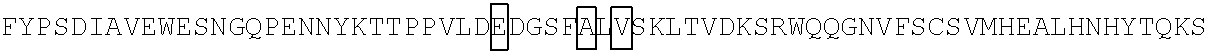

LSLSPGKGGGGSGGGGSGGGGSIWELKKDVYVVELDWYPDAPGEMVVLTCDTPEEDGITWTLDQSSEVLGSG

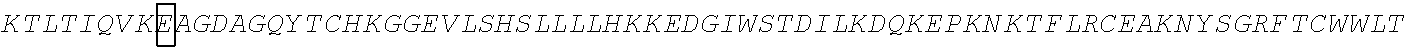

TSSFFIRDIKPDPPKNLQLKPLKNSRQVEVSWEYPDTWSTPHSYFSLTFCVQVQGKSKREKKDRVFTDKTSATVIC
RKNASISVRAQDRYYSSSWSEWASVPCSGGGGSGGGGSGGGGSGGGGSGRNLPVATPDPGMFPCLHHSQNLL
RAVSNMLQKARQTLEFYPCTSEEIDHEDITKDKTSTVEACLPLELTKNESCLNSRETSFITNGSCLASRKTSEMMAL
CLSSIYEDLKMYQVEFKTMNAKLLMDPKRQIFLDQNMLAVIDELMQALNENSETVPQKSSLEEPDFYKTKIKLCIL
LHAFRIRAVTIDRVMSYLNAS
SEQ ID NO: 21 (anti-PD-1 Ab VH-CH1-IgG1 Fc mutant2; VH is underlined; hinge is bolded; linker is
bolded and
underlined)QVQLVESGGGVVQPGRSLRLDCKASGITESNSGMHWVRQAPGKGLEWVAVIWYDGSKRYYA
DSVKGRFTISRDNSKNTLFLQMNSLRAEDTAVYYCATNDDYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS

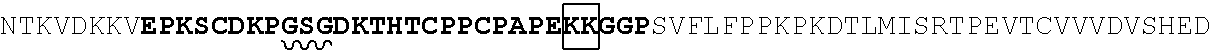

PEVKFNWYVDGVEVHNAKTKPREEQYNSTYRVVSVLTVLHQDWLNGKEYKCKVSNKALPAPIEKTISKA

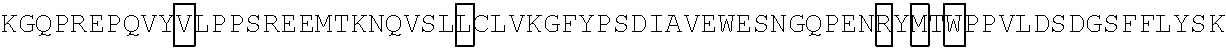

LTVDKSRWQQGNVFSCSVMHEALHNHYTQKSLSLSPGK
SEQ ID NO: 22 (anti-PD-1 Ab VH-CHI-N' hinge portion-linker-single-chain IL-12 mutant heterodimer IL-
12B (p40 ES9A/F60A)-linker-IL-12A (wt p35)-C' hinge portion-IgG1 Fc mutant1; VH is underlined; hinge is
bolded; linker is bolded and underlined; IL-12 subunits are italicized)
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

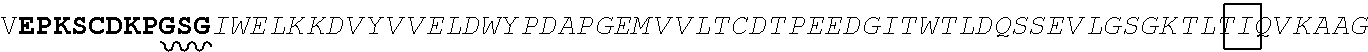

DAGQYTCHKGGEVLSHSLLLLHKKEDGIWSTDILKDQKEPKNKTFLRCEAKNYSGRFTCWWLTTISTDLTESVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSONLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNENSETVPQKSSLEEPDFYKTKIKLCILLHAFRIRAVTID

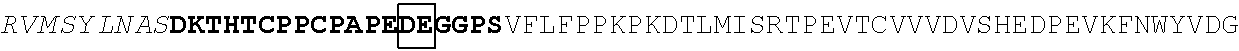

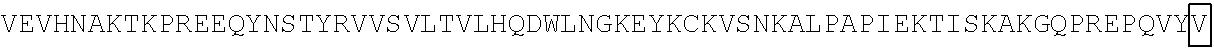

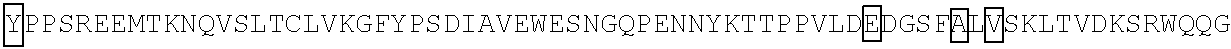

NVFSCSVMHEALHNHYTQKSLSLSPGK
SEQ ID NO: 23 (anti-PD-1 Ab VH-CH1-N' hinge portion-linker-single-chain IL-12 mutant heterodimer IL-
12B (p40 F60A)-linker-IL-12A (wt p35)-C' hinge portion-IgG1 Fc mutant1; VH is underlined; hinge is
bolded; linker is bolded and underlined; IL-12 subunits are italicized)
QVQLVESGGGVVOPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

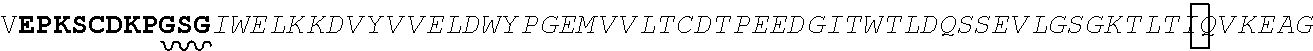

DAGQYTCHKGGEVLSHSLLLLHKKEDGIWSTDILKDQKEPKNKTFLRCEAKNYSGRFTCWWLTTISTDLTESVKSS
RGSSDPQGVTCGAATLSAERVRGDNKEYEYSVECQEDSACPAAEESLPIEVMVDAVHKLKYENYTSSFFIRDIIKPD
PPKNLOLKPLKNSRQVEVSWEYPDTWSTPHSYFSLTFCVQVQGKSKREKKDRVFTDKTSATVICRKNASISVRAOD
RYYSSSWSEWASVPCSGGGGSGGGGSGGGGSGGGGSGRNLPVATPDPGMFPCLHHSQNLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNENSETVPQKSSLEEPDFYKTKIKLCILLHAFRIRAVTID

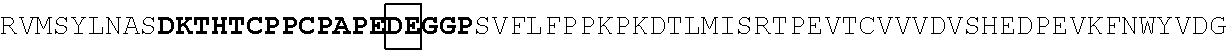

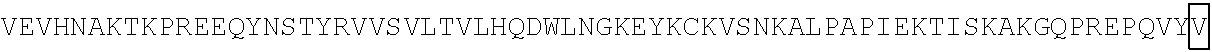

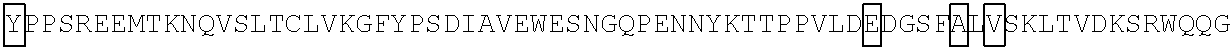

NVFSCSVMHEALHNHYTQKSLSLSPGK
SEQ ID NO: 24 (wildtype human PD-L2 extracellular domain-linker-IL-2 mutant R38D/K43E/E61R-hinge-
IgG1 Fc mutant1; PD-L2 is underlined; linker is bolded and underlined; hinge is bolded; IL-2 mutant is
italicized)
LFTVTVPKELYIIEHGSNVTLECNFDTGSHVNLGAITASLQKVENDTSPHRERATLLEEQLPLGKASHIPQV
QVRDEGQYQCIIIYGVAWDYKYLTLKVKASYRKINTHILKVPETDEVELTCQATGYPLAEVSWPNVSVPAN
TSHSRTPEGLYOVTSVLRLKPPPGRNFSCVFWNTHVRELTLASIDLQSQMEPRTHPTGGGGSAPTSSSTKKT

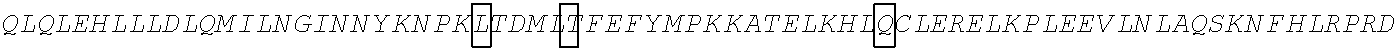

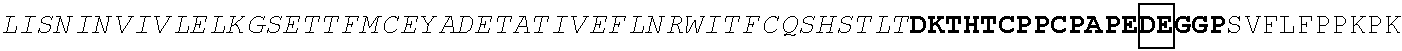

DTLMISRTPEVTCVVVDVSHEDPEVKFNWYVDGVEVHNAKTKPREEQYNSTYRVVSVLTVLHQDWLNGK

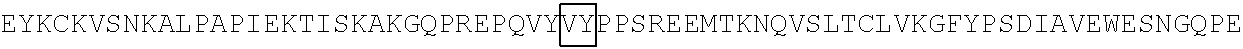

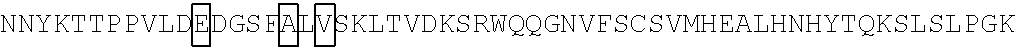

SEQ ID NO: 25 (wildtype mature human IL-2)
APTSSSTKKTQLQLEHLLLDLQMILNGINNYKNPKLTRMLTFKFYMPKKATELKHLQCLEEELKPLEEVLN
LAQSKNFHLRPRDLISNINVIVLELKGSETTFMCEYADETATIVEFLNRWITFCQSIISTLT
SEQ ID NO: 26 (IL-2 mutant1 R38D/K43E/E61R)

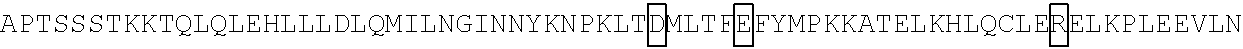

LAQSKNFHLRPRDLISNINVIVLELKGSETTFMCEYADETATIVEFLNRWITFCQSIISTLT
SEQ ID NO: 27 (IL-2 mutant2 L18R/Q22E/R38D/K43E/E61R)

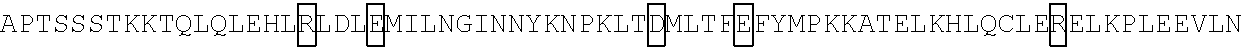

LAQSKNFHLRPRDLISNINVIVLELKGSETTFMCEYADETATIVEFLNRWITFCQSHISTLT
SEQ ID NO: 28 (IL-2 mutant3 R38D/K43E/E61R/Q126T)

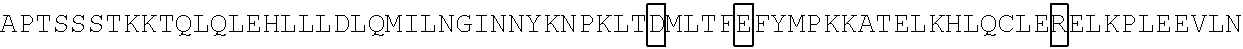

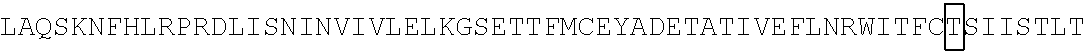

SEQ ID NO: 29 (IL-2 mutant4 L18R/Q22E/R38D/K43E/E61R/Q126T)

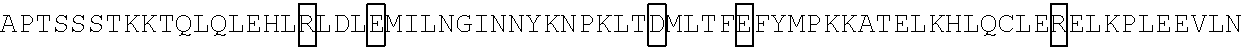

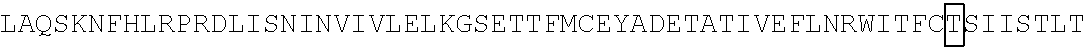

SEQ ID NO: 30 (IL-2 mutant5 L18R/Q22E/R38D/K43E/E61R/Q126T/S130R)

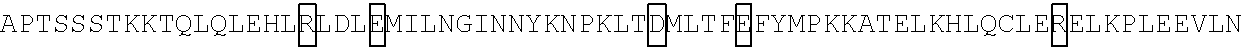

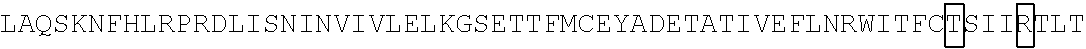

SEQ ID NO: 31 (wildtype mature human IFN-α2b)
CDLPQTHSLGSRRTLMLLAQMRKISLFSCLKDRHDFGFPQEEFGNQFQKAETIPVLHEMIQQIFNLFSTKDSS
AAWDETLLDKFYTELYQQLNDLEACVIQGVGVTETPLMKEDSILAVRKYFQRITLYLKEKKYSPCAWEVV
RAEIMRSFSLSTNLQESLRSKE
SEQ ID NO: 32 (IFN-α2b mutant L30A)

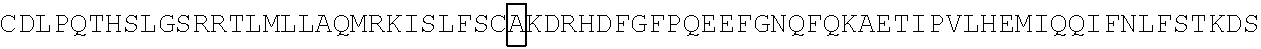

SAAWDETLLDKFYTELYQQLNDLEACVIQGVGVTETPLMKEDSILAVRKYFORITLYLKEKKYSPCAWEV
VRAEIMRSFSLSTNLQESLRSKE
SEQ ID NO: 33 (IFN-α2b mutant K31A)

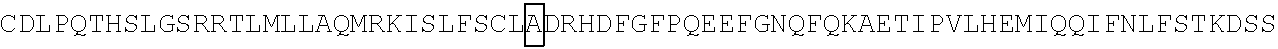

AAWDETLLDKFYTELYQQLNDLEACVIQGVGVTETPLMKEDSILAVRKYFQRITLYLKEKKYSPCAWEVV
RAEIMRSFSLSTNLQESLRSKE
SEQ ID NO: 34 (IFN-α2b mutant D32A)

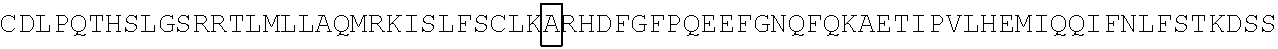

AAWDETLLDKFYTELYQQLNDLEACVIQGVGVTETPLMKEDSILAVRKYFORITLYLKEKKYSPCAWEVV
RAEIMRSFSLSTNLQESLRSKE
SEQ ID NO: 35 (IFN-α2b mutant R33A)

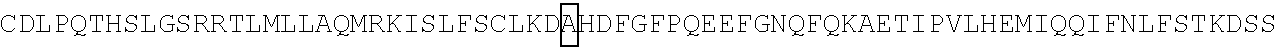

AAWDETLLDKFYTELYQQLNDLEACVIQGVGVTETPLMKEDSILAVRKYFQRITLYLKEKKYSPCAWEVV
RAEIMRSFSLSTNLQESLRSKE
SEQ ID NO: 36 (IFN-α2b mutant H34A)

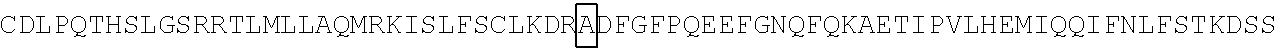

AAWDETLLDKFYTELYQQLNDLEACVIQGVGVTETPLMKEDSILAVRKYFQRITLYLKEKKYSPCAWEVV
RAEIMRSFSLSTNLQESLRSKE
SEQ ID NO: 37 (IFN-α2b mutant D35A)

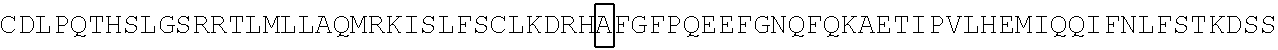

AAWDETLLDKFYTELYQQLNDLEACVIQGVGVTETPLMKEDSILAVRKYFORITLYLKEKKYSPCAWEVV
RAEIMRSFSLSTNLQESLRSKE
SEQ ID NO: 38 (wildtype mature human IFN-γ monomer)
QDPYVKEAENLKKYFNAGHSDVADNGTLFLGILKNWKEESDRKIMQSQIVSFYFKLFKNFKDDQSIQKSVE
TIKEDMNVKFFNSNKKKRDDFEKLTNYSVTDLNVQRKAIHELIQVMAELSPAAKTGKRKRSQMLFRG
SEQ ID NO: 39 (IFN-γ mutant S20A/D21A monomer)

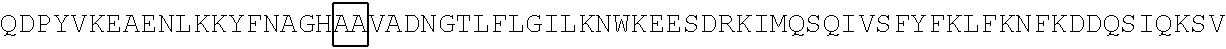

ETIKEDMNVKFFNSNKKKRDDFEKLTNYSVTDLNVQRKAIHELIQVMAELSPAAKTGKRKRSQMLFRG
SEQ ID NO: 40 (IFN-γ mutant V22A/A23S monomer)

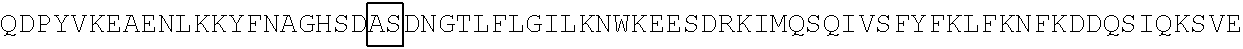

TIKEDMNVKFFNSNKKKRDDFEKLTNYSVTDLNVQRKAIHELIQVMAELSPAAKTGKRKRSQMLFRG
SEQ ID NO: 41 (IFN-γ mutant A23V monomer)

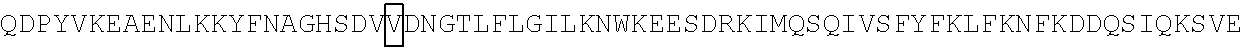

TIKEDMNVKFFNSNKKKRDDFEKLTNYSVTDLNVQRKAIHELIQVMAELSPAAKTGKRKRSQMLFRG
SEQ ID NO: 42 (IFN-γ mutant D24A/N25A monomer)

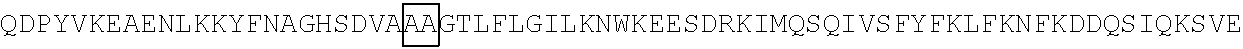

TIKEDMNVKFFNSNKKKRDDFEKLTNYSVTDLNVQRKAIHELIQVMAELSPAAKTGKRKRSQMLFRG
SEQ ID NO: 43 (IFN-γ mutant A23E/D24E/N25K monomer)

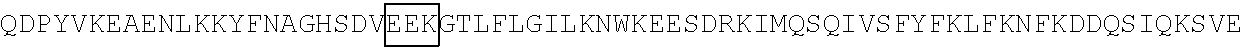

TIKEDMNVKFFNSNKKKRDDFEKLTNYSVTDLNVQRKAIHELIQVMAELSPAAKTGKRKRSQMLFRG
SEQ ID NO: 44 (IFN-γ mutant A23Q monomer)

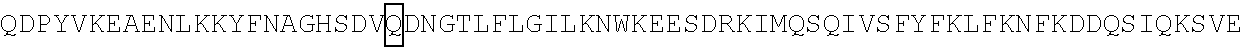

TIKEDMNVKFFNSNKKKRDDFEKLTNYSVTDLNVQRKAIHELIQVMAELSPAAKTGKRKRSQMLFRG
SEQ ID NO: 45 (IFN-γ mutant D21K monomer)

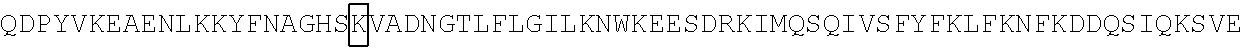

TIKEDMNVKFFNSNKKKRDDFEKLTNYSVTDLNVQRKAIHELIQVMAELSPAAKTGKRKRSQMLFRG
SEQ ID NO: 46 (single-chain ″wildtype″ IFN-7 homodimer; linker is bolded; wildtype IFN-γ monomer is
italicized)
QDPYVKEAENLKKYFNAGHSDVADNGTLFLGILKNWKEESDRKIMQSQIVSFYFKLFKNFKDDQSIQKSVETIKE
DMNVKFFNSNKKKRDDFEKLINYSVTDLNVQRKAIHELIQVMAELSPAAKTGKRKRSQMLERGFEGGGSGGGG
SGGGGSGGGGSQDPYVKEAENLKKYFNAGHSDVADNGTLFLGILKNWKEESDRKIMQSQIVSFYFKLFKNEKD
DQSIQKSVETIKEDMNVKFFNSNKKKRDDFEKLTNYSVTDLNVQRKAIHELIQVMAELSPAAKTGKRKRSQMLFR
G
SEQ ID NO: 47 (single-chain IFN-γ mutant A23V homodimer; linker is bolded; IFN-γ mutant monomer is
italicized)

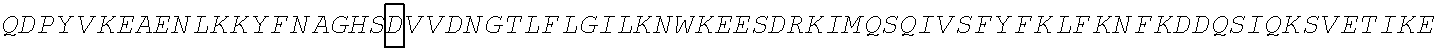

DMNVKFFNSNKKKRDDFEKLTNYSVTDLNVQRKAIHELIQVMAELSPAAKTGKRKRSQMLFRGFEGGGSGGGG

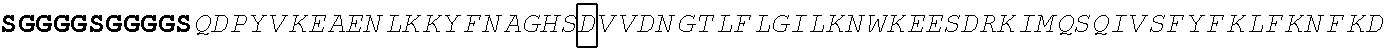

DQSIQKSVETIKEDMNVKFFNSNKKKRDDFEKLINYSVTDLNVQRKAIHELIQVMAELSPAAKTGKRKRSQMLFR
G
SEQ ID NO: 48 (nivolumab/Opdivo anti-PD-1 Ab VH)
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSAS
SEQ ID NO: 49 (nivolumab/Opdivo anti-PD-1 Ab VL)
EIVLTQSPATLSLSPGERATLSCRASQSVSSYLAWYQQKPGQAPRLLIYDASNRATGIPARFSGSGSGTDFTL
TISSLEPEDFAVYYCQQSSNWPRTFGQGTKVEI
SEQ ID NO: 50 (nivolumab/Opdivo anti-PD-1 Ab LC; VL is underlined)
EIVLTQSPATLSLSPGERATLSCRASOSVSSYLAWYQQKPGQAPRLLIYDASNRATGIPARFSGSGSGTDETL
TISSLEPEDFAVYYCQQSSNWPRTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
SEQ ID NO: 51 (anti-PD-1 Ab HC (IgG1 Fc mutant2); VH is underlined; hinge is bolded)
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

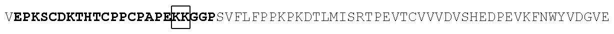

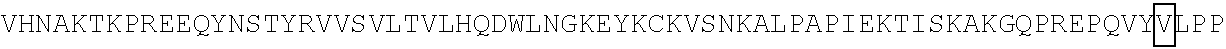

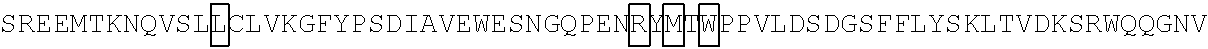

FSCSVMHEALHNHYTQKSLSLSPGK
SEQ ID NO: 52 (wildtype mature human IL-10 monomer)
SPGQGTQSENSCTHFPGNLPNMLRDLRDAFSRVKTFFQMKDQLDNLLLKESLLEDFKGYLGCQALSEMIQF
YLEEVMPQAENQDPDIKAHVNSLGENLKTLRLRLRRCHRFLPCENKSKAVEQVKNAFNKLQEKGIYKAMS
EFDIFINYIEAYMTMKIRN
SEQ ID NO: 53 (IL-10 mutant R24A monomer)

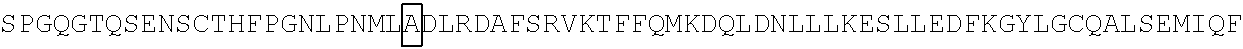

YLEEVMPQAENQDPDIKAHVNSLGENLKTLRLRLRRCHRFLPCENKSKAVEQVKNAFNKLQEKGIYKAMS
EFDIFINYIEAYMTMKIRN
SEQ ID NO: 54 (IL-10 mutant D25A/L26A monomer)

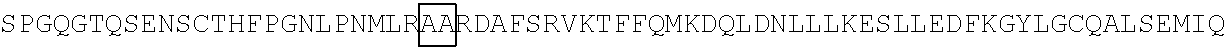

FYLEEVMPQAENQDPDIKAHVNSLGENLKTLRLRLRRCHRFLPCENKSKAVEQVKNAFNKLQEKGIYKAM
SEFDIFINYIEAYMTMKIRN
SEQ ID NO: 55 (IL-10 mutant R27A monomer)

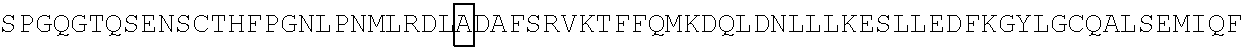

YLEEVMPQAENQDPDIKAHVNSLGENLKTLRLRLRRCHRFLPCENKSKAVEQVKNAFNKLQEKGIYKAMS
EFDIFINYIEAYMTMKIRN
SEQ ID NO: 56 (IL-10 mutant D28A/A29S monomer)

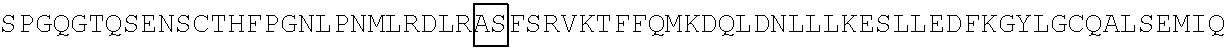

YLEEVMPQAENQDPDIKAHVNSLGENLKTLRLRLRRCHRFLPCENKSKAVEQVKNAFNKLQEKGIYKAMS
EFDIFINYJEAYMTMKIRN
SEQ ID NO: 57 (IL-10 mutant F30A/S31A monomer)

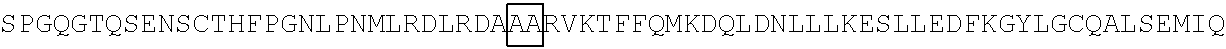

FYLEEVMPQAENQDPDIKAHVNSLGENLKTLRLRLRRCHRFLPCENKSKAVEQVKNAFNKLQEKGIYKAM
SEFDIFINYIEAYMTMKIRN
SEQ ID NO: 58 (IL-10 mutant R32A monomer)

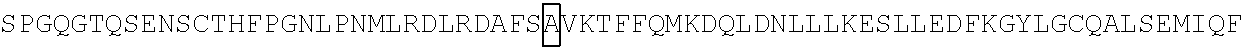

YLEEVMPQAENQDPDIKAHVNSLGENLKTLRLRLRRCHRFLPCENKSKAVEQVKNAFNKLQEKGIYKAMS
EFDIFINYIEAYMTMKIRN
SEQ ID NO: 59 (single-chain ″wildtype″ IL-10 homodimer; linker is bolded; wildtype IL-10 monomer is
italicized)
SPGQGTQSENSCTHFPGNLPNMLRDLRDAFSRVKTFFQMKDQLDNLLLKESLLEDFKGYLGCQALSEMIQFYLE
EVMPQAENQDPDIKAHVNSLGENLKTLRLRLRRCHRFLPCENKSKAVEQVKNAFNKLQEKGIYKAMSEFDIFINYI
EAYMTMKIRNFEGGGSGGGGSGGGGSGGGGSSPGQGTQSENSCTHFPGNLPNMLRDLRDAFSRVKTFFQMK
DQLDNLLLKESLLEDFKGYLGCQALSEMIQFYLEEVMPQAENQDPDIKAHVNSLGENLKTLRLRLRRCHRFLPCE
NKSKAVEQVKNAFNKLQEKGIYKAMSEFDIFINYIEAYMTMKIRN
SEQ ID NO: 60 (single-chain IL-10 mutant R27A homodimer; linker is bolded; IL-10 mutant monomer is
italicized)

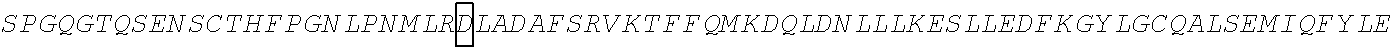

EVMPQAENQDPDIKAHVNSLGENLKTLRLRLRRCHRFLPCENKSKAVEQVKNAFNKLOEKGIYKAMSEFDIFINYI

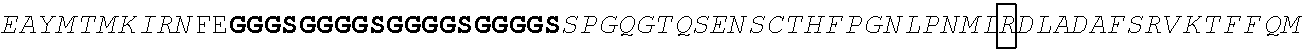

KDQLDNLLLKESLLEDFKGYLGCQALSEMIQFYLEEVMPQAENQDPDIKAHVNSLGENLKTLRLRLRRCHRFLPC
ENKSKAVEQVKNAFNKLQEKGIYKAMSEFDIFINYIEAYMTMKIRN
SEQ ID NO: 61 (wildtype mature human IL-12A (p35) subunit)
RNLPVATPDPGMFPCLHHSQNLLRAVSNMLQKARQTLEFYPCTSEEIDHEDITKDKTSTVEACLPLELTKNE
SCLNSRETSFITNGSCLASRKTSFMMALCLSSIYEDLKMYQVEFKTMNAKLLMDPKRQIFLDQNMLAVIDE
LMQALNFNSETVPQKSSLEEPDFYKTKIKLCILLHAFRIRAVTIDRVMSYLNAS
SEQ ID NO: 62 (wildtype mature human IL-12B (p40) subunit)
IWELKKDVYVVELDWYPDAPGEMVVLTCDTPEEDGITWTLDQSSEVLGSGKTLTIQVKEFGDAGQYTCHK
GGEVLSHSLLLLHKKEDGIWSTDILKDQKEPKNKTFLRCEAKNYSGRFTCWWLTTISTDLTFSVKSSRGSSD
PQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASIS
VRAQDRYYSSSWSEWASVPCS
SEQ ID NO: 63 (IL-12B (p40) mutant E59A/F60A sabunit)

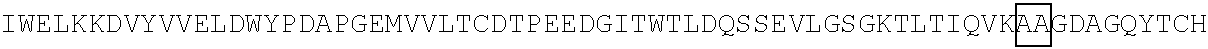

KGGEVLSHSLLLLHKKEDGIWSTDILKDQKEPKNKTFLRCEAKNYSGRFTCWWLTTISTDLTFSVKSSRGSS
DPQGVTCGAATLSAER VRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
SEQ ID NO: 64 (IL-12B (p40) mutant E59A subunit)

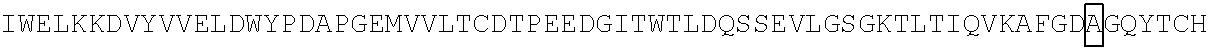

KGGEVLSHSLLLLHKKEDGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
SEQ ID NO: 65 (IL-12B (p40) mutant F60A subunit)

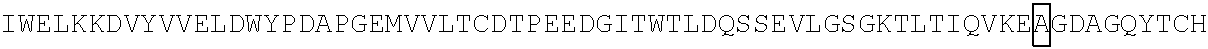

KGGEVLSHSLLLLHKKEDGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
SEQ ID NO: 66 (IL-12B (p40) mutant G64A subunit)

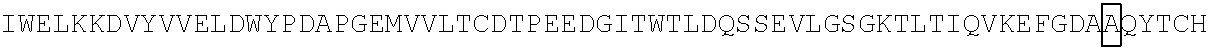

KGGEVLSHSLLLLHKKEDGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
SEQ ID NO: 67 (single-chain ″wildtype″ IL-12 heterodimer IL-12B (wt p40)-linker-IL-12A (wt p35); linker
is bolded)
IWELKKDVYVVELDWYPDAPGEMVVLTCDTPEEDGITWTLDQSSEVLGSGKTLTIQVKEFGDAGQYTCHKGGEV
LSHSLLLLHKKEDGIWSTDILKDQKEPKNKTFLRCEAKNYSGRFTCWWLTTISTDLTFSVKSSRGSSDPOGVTCGA
ATLSAERVRGDNKEYEYSVECQEDSACPAAEESLPIEVMVDAVHKLKYENYTSSFFIRDIIKPDPPKNLQLKPLKNS
RQVEVSWEYPDTWSTPHSYFSLTFQVQVQGKSKREKKDRVFTDKTSATVICRKNASISVRAQDRYYSSSWSEWASV
PCSGGGGSGGGGSGGGGSGGGGSGRNLPVATPDPGMFPCLHHSQNLLRAVSNMLQKARQTLEFYPCTSEEI
DHEDITKDKISTVEACLPLELTKNESCLNSRETSFITNGSCLASRKTSEMMALCLSSIYEDLKMYQVEFKIMNAKLL
MDPKRQIFLDQNMLAVIDELMQALNENSETVPQKSSLEEPDFYKTKIKLCILLHAFRIRAVTIDRVMSYLNAS
SEQ ID NO: 68 (single-chain IL-12 mutant heterodimer IL-12B (p40 E59A/F60A)-linker-IL-12A (wt p35);
linker is bolded; IL-12 subunits are italicized)

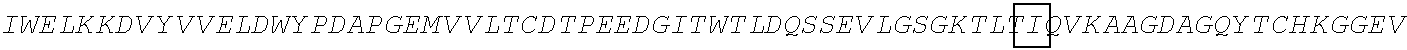

LSHSLLLLHKKEDGIWSTDILKDQKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLOLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGGSGGGGSGGGGSGRNLPVATPDPGMFPCLHHSQNLLRAVSNMLQKARQTLEFYPCTSEEI
DHEDITKDKTSTVEACLPLELTKNESCLNSRETSFITNGSCLASRKTSFMMALCLSSIYEDLKMYQVEFKTMNAKLL
MDPKROIFLDQNMLAVIDELMQALNENSETVPOKSSLEEPDFYKTKIKLCILLHAFRIRAVTIDRVMSYLNAS
SEQ ID NO: 69 (single-chain IL-12 mutant heterodimer IL-12B (p40 E59A)-linker-IL-12A (wt p35); linker is
bolded and underlined; IL-12 subunits are italicized)

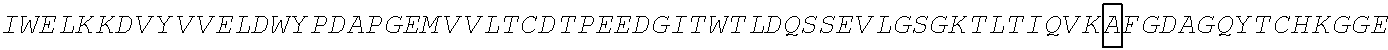

VLSHSLLLLHKKEDGIWSTDILKDQKEPKNKTFLRCEAKNYSGRFTCWWLTTISTDLTFSVKSSRGSSDPQGVTCG
AATLSAERVRGDNKEYEYSVECQEDSACPAAEESLPIEVMVDAVHKLKYENYTSSFFIRDIIKPDPPKNLQLKPLKN
SRQVEVSWEYPDTWSTPHSYFSLTFCVQVQGKSKREKKDRVFTDKTSATVICRKNASISVRAQDRYYSSSWSEWAS
VPCSGGGGSGGGGSGGGGSGGGGSGRNLPVATPDPGMFPCLHHSQNLLRAVSNMLQKARQTLEFYPCTSEE
IDHEDITKDKTSTVEACLPLELTKNESCLNSRETSFITNGSCLASRKTSFMMALCLSSIYEDLKMYOVEFKTMNAKL
LMDPKRQIFLDQNMLAVIDELMQALNENSETVPQKSSLEEPDFYKTKIKLCILLHAFRIRAVTIDRVMSYLNAS
SEQ ID NO: 70 (single-chain IL-12 mutant heterodimer IL-12B (p40 G64A)-linker-IL-12A (wt p35); linker
is bolded and underlined; IL-12 subunits are italicized)

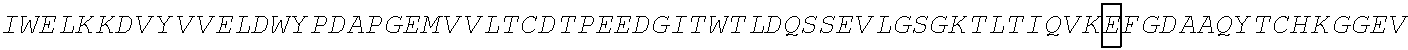

LSHSLLLLHKKEDGIWSTDILKDOKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLQLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGGSGGGGSGGGGSGRNLPVATPDPGMFPCLHHSQNLLRAVSNMLQKARQTLEFYPCTSEEI
DHEDITKDKTSTVEACLPLELTKNESCLNSRETSFITNGSCLASRKTSEMMALCLSSIYEDLKMYOVEFKTMNAKLL
MDPKRQIFLDQNMLAVIDELMQALNENSETVPOKSSLEEPDFYKTKIKLCILLHAFRIRAVTIDRVMSYLNAS
SEQ ID NO: 71 (single-chain IL-12 mutant heterodimer IL-12B (p40 F60A)-linker-IL-12A (wt p35); linker
is bolded; IL-12 subunits are italicized)

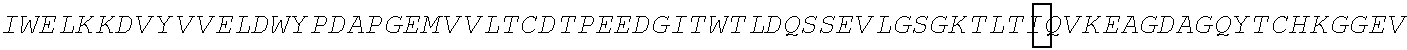

LSHSLLLLHKKEDGIWSTDILKDQKEPKNKTFLRCEAKNYSGRFTCWWLTTISTDLTFSVKSSRGSSDPOGVTCGA
ATLSAERVRGDNKEYEYSVECQEDSACPAAEESLPIEVMVDAVHKLKYENYTSSFFIRDIIKPDPPKNLQLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGGSGGGGSGGGGSGRNLPVATPDPGMFPCLHHSONLLRAVSNMLQKAROTLEFYPCTSEEI
DHEDITKDKTSTVEACLPLELTKNESCLNSRETSFITNGSCLASRKTSEMMALCLSSIYEDLKMYQVEFKTMNAKLL
MDPKRQIFLDQNMLAVIDELMQALNFNSETVPQKSSLEEPDFYKTKIKLCILLHAFRIRAVTIDRVMSYLNAS
SEQ ID NO: 72 (mouse single-chain mutant heterodimer IL-12B (E59A/F60A)-linker-IL-12A (wt p35); linker
is bolded and underlined; mouse IL-12 subunits are italicized)

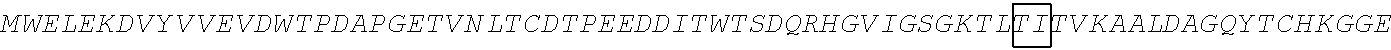

TLSHSHLLLHKKENGIWSTEILKNEKNKTFLKCEAPNYSGRFTCSWLVQRNMDLKFNIKSSSSSPDSRAVTCGMAS
LSAEKVTLDQRDYEKYSVSCQEDVTCPTAEETLPIELALEARQQNKYENYSTSFFIRDIIKPDPPKNLQMKPLKNSQ
VEVSWEYPDSWSTPHSYFSLKFFVRIQRKKEKMKETEEGCNQKGAFLVEKTSTEVQCKGGNVCVQAQDRYYNSS
CSKWACVPCRVRSGGPGGGGSGGGSGGGGSGRVIPVSGPARCLSQSRNLLKTTDDMVKTAREKLKHYSCTAE
DIDHEDITRDQTSTLKTCLPLELHKNESCLATRETSSTTRGSCLPPQKTSLMMTLCLGSIYEDLKMYQTEFQAINAA
LQNHNHQQIILDKGMLVAIDELMQSLNHNGETLRQKPPVGEADPYRVKMKLCILLHAFSTRVVTINRVMGYLSSA
SEQ ID NO: 73 (wildtype mature human IL-23A (p19) subunit)
RAVPGGSSPAWTQCQQLSQKLCTLAWSAHPLVGHMDLREEGDEETTNDVPHIQCGDGCDPQGLRDNSQF
CLQRIHQGLIFYEKLLGSDIFTGEPSLLPDSPVGQLHASLLGLSQLLQPEGHHWETQQIPSLSPSQPWQRLLL
RFKILRSLQAFVAVAARVFAHGAATLSP
SEQ ID NO: 74 (single-chain ″wildtype″ IL-23 heterodimer IL-12B (wt p40)-linker-IL-23A (wt p19); linker
is bolded; IL-23 subunits are italicized)
IWELKKDVYVVELDWYPDAPGEMVVLTCDTPEEDGITWTLDQSSEVLGSGKTLTIQVKEFGDAGQYTCHKGGEV
LSHSLLLLHKKEDGIWSTDILKDQKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLOLKPLKNS
RQVEVSWEYPDTWSTPHSYFSLTFCVQVQGKSKREKKDRVFTDKTSATVICRKNASISVRAODRYYSSSWSEWASV
PCSGGGGSGGGGSGGGGSGGGGSGRAVPGGSSPAWTQCQQLSQKLCTLAWSAHPLVGHMDLREEGDEETT
NDVPHIQCGDGCDPQGLRDNSQFCLQRIHQGLIFYEKLLGSDIFTGEPSLLPDSPVGQLHASLLGLSOLLOPEGH
HWETQQIPSLSPSQPWORLLLRFKILRSLQAFVAVAARVFAHGAATLSP
SEQ ID NO: 75 (single-chain IL-23 mutant heterodimer IL-12B (p40 E59A/F60A)-linker-IL-23A (wt p19);
linker is bolded; IL-23 subunits are italicized)

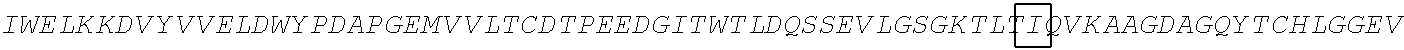

LSHSLLLLHKKEDGIWSTDILKDQKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLQLKPLKNS
RQVEVSWEYPDTWSTPHSYFSLIFCVQVQGKSKREKKDRVFTDKTSATVICRKNASISVRAQDRYYSSSWSEWASV
PCSGGGGSGGGGSGGGGSGGGGSGRAVPGGSSPAWTQCQQLSQKLCTLAWSAHPLVGHMDLREEGDEETT
NDVPHIQCGDGCDPQGLRDNSQFCLORIHQGLIFYEKLLGSDIFTGEPSLLPDSPVGQLHASLLGLSOLLQPEGH
HWETQQIPSLSPSQPWQRLLLRFKILRSLQAFVAVAARVFAHGAATLSP
SEQ ID NO: 76 (hinge)
EPKSCDKTHTCPPCPAPELLGGP
SEQ ID NO: 77 (hinge)

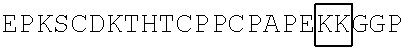

SEQ ID NO: 78 (hinge)

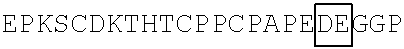

SEQ ID NO: 79 (hinge)

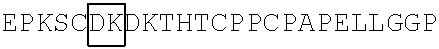

SEQ ID NO: 80 (hinge)

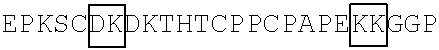

SEQ ID NO: 81 (hinge)

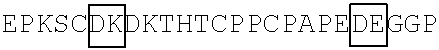

SEQ ID NO: 82 (hinge)
ERKCCVECPPCPAPPVAGP
SEQ ID NO: 83 (hinge)
ESKYGPPCPSCPAPEFLGGP
SEQ ID NO: 84 (hinge, e.g., hinge N' portion)
EPKSCDK
SEQ ID NO: 85 (hinge, e.g., hinge N' portion)
EPKSC
SEQ ID NO: 86 (hinge, e.g., hinge C' portion)
DKTHTCPPCPAPELLGGP
SEQ ID NO: 87 (hinge, e.g., hinge C' portion)

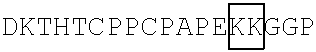

SEQ ID NO: 88 (hinge, e.g., hinge C' portion)

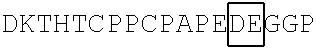

SEQ ID NO: 89 (hinge)
DKTHT
SEQ ID NO: 90 (hinge, e.g., hinge N' portion)

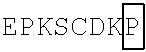

SEQ ID NO: 91 (hinge)

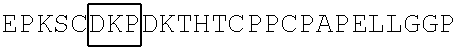

SEQ ID NO: 92 (hinge)

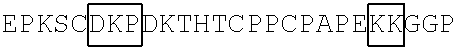

SEQ ID NO: 93 (hinge)

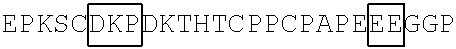

SEQ ID NO: 94 (hinge)
ESKYGPPCPPCPAPEFLGGP
SEQ ID NO: 95 (hinge)

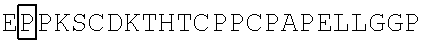

SEQ ID NO: 96 (wildtype human IgG1 Fc)
SVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGK
SEQ ID NO: 97 (IgG1 Fc mutant1 T350V/L351Y/S400E/F405A/Y407V)
SVFLFPPKPKDTLMISRTPEVTCVVVDVSHEDPEVKFNWYVDGVEVHNAKTKPREEQYNSTYRVVSVLTV

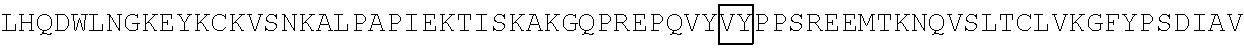

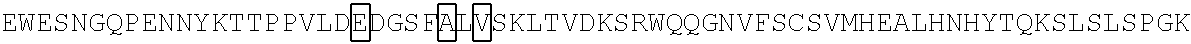

SEQ ID NO: 98 (IgG1 Fc mutant2 T350V/T366L/N390R/K392M/T394W)
SVFLFPPKPKDTLMISRTPEVTCVVVDVSHEDPEVKFNWYVDGVEVHNAKTKPREEQYNSTYRVVSVLTV

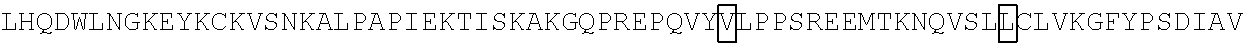

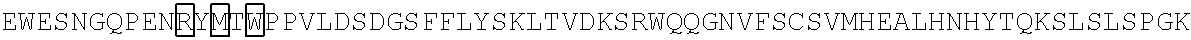

SEQ ID NO: 99 (wildtype human IgG4 Fc)
SVFLFPPKPKDTLMISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLGK
SEQ ID NO: 100 (IgG1 Fc mutant)

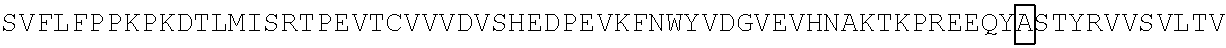

LHQDWLNGKEYKCKVSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALHNHYTQKSLSLSPGK
SEQ ID NO: 101 (IgG1 Fc mutant)

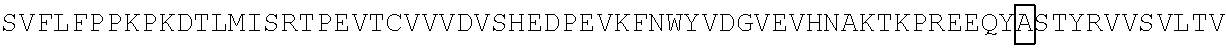

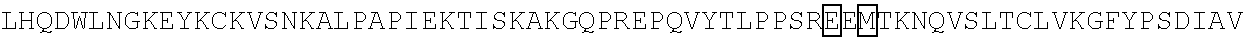

EWESNGQPENNYKTTPPVLDSDGSFFLYSKLTVDKSRWQQGNVFSCSVMHEALHNHYTQKSLSLSPGK
SEQ ID NO: 102 (IgG1 Fc mutant)
SVFLFPPKPKDTLMISRTPEVTCVVVDVSHEDPEVKFNWYVDGVEVHNAKTKPREEQYNSTYRVVSVLTV

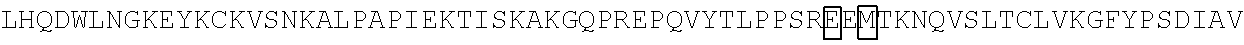

EWESNGQPENNYKTTPPVLDSDGSFFLYSKLTVDKSRWQQGNVFSCSVMHEALHNHYTQKSLSLSPGK
SEQ ID NO: 103 (anti-PD-1 Ab HC (IgG1 Fc mutant); VH is underlined)
QVQLVESGGGVVQPGRSLRLDCKASGITESNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEV
HNAKTKPREEQYASTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 104 (nivolumab/Opdivo anti-PD-1 Ab HC; VH is underlined; hinge is bolded)
QVQLVESGGGVVQPGRSLRLDCKASGITFSNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKR
VESKYGPPCPPCPAPEFLGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQEGNVFSCSVM
HEALHNHYTQKSLSLSLGK
SEQ ID NO: 105 (wildtype human PD-L2; signal peptide is italicized; extracellular domain is
underlined; cytoplasmic domain is bolded)
MIFLLLMLSLELQLHQIAALFTVTVPKELYIIEHGSNVTLECNEDTGSHVNLGAITASLQKVENDTSPHRERAT
LLEEQLPLGKASFHIPQVQVRDEGQYQCIIIGVAWDYKYLTLKVKASYRKINTHILKVPETDEVELTCQAT
GYPLAEVSWPNVSVPANTSHSRTPEGLYQVTSVLRLKPPPGRNFSCVFWNTHVRELTLASIDLQSQMEPRT
HPTWLLHIFIPFCHIAFIFIATVIALRKQLCQKLYSSKDTTKRPVTTTKREVNSAI
SEQ ID NO: 106 (wildtype human PD-L2 extracellular domain)
LFTVTVPKELYJEHGSNVTLECNFDTGSHVNLGAITASLOKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPT
SEQ ID NO: 107 (human PD-L2 extracellular domain mutant1 T56V)

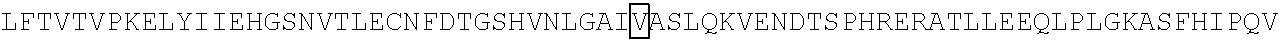

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPT
SEQ ID NO: 108 (human PD-L2 extracellular domain motant2 S58V)

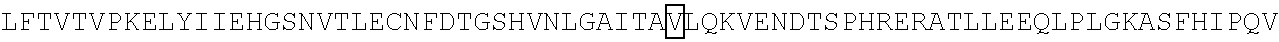

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPT
SEQ ID NO: 109 (human PD-L2 extracellular domain mutant3 Q60L)

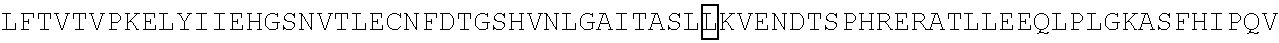

QVRDEGQYQCHIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPT
SEQ ID NO: 110 (human PD-L2 extracellular domain mutant4 T56V/S58V/Q60L)

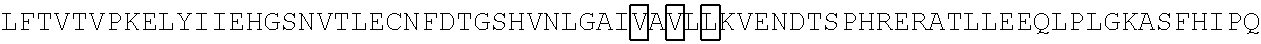

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPT
SEQ ID NO: 111 (wildtype human PD-L2 extracellular domain-hinge-IgG1 Fc mutant; PD-L2 extracellular
domain is underlined; hinge is bolded)
LFTVTVPKELYIIEHGSNVTLECNEDTGSHVNLGAITASLQKVENDTSPHRERATLLEEQLPLGKASFHIPOV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTDKTHTCPPCPAPEL
LGGPSVFLFPPKPKDTLMISRTPEVTCVVVDVSHEDPEVKFNWYVDGVEVHNAKTKPREEQYASTYRVVS
VLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSP
GK
SEQ ID NO: 112 (wildtype human PD-L2 extracellular domain-hinge-IgG1 Fc mutant2; PD-L2 extracellular
domain is underlined; hinge is bolded)
LFTVTVPKELYIIEHGSNVTLECNEDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN

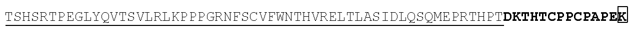

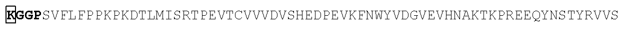

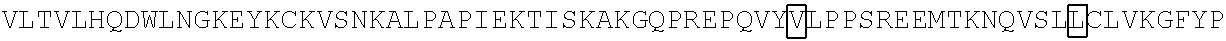

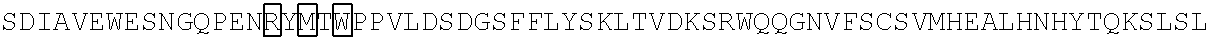

SPGK
SEQ ID NO: 113 (wildtype human PD-L2 extracellular domain-hinge-IgG1 Fc mutant2; PD-L2 extracellular
domain is underlined; linker is bolded and underlined; hinge is bolded)
LFTVTVPKELYIIEHGSNVTLECNEDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSDKTHTCPPC

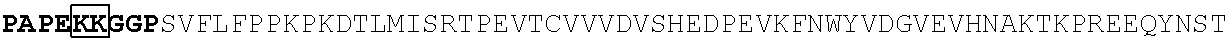

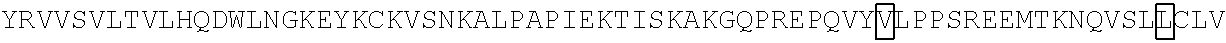

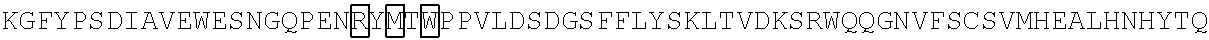

KSLSLSPGK
SEQ ID NO: 114 (wildtype human PD-L2 extracellular domain-linker-hinge-IgG1 Fc mutant2; PD-L2
extracellular domain is underlined; linker is bolded and underlined; hinge is bolded)
LFTVTVPKELYIIEHGSNVTLECNEDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGGGGGDKTHTC

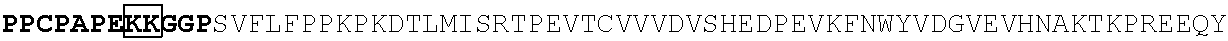

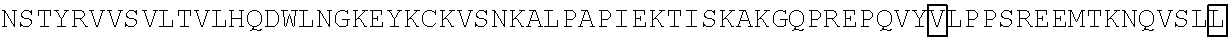

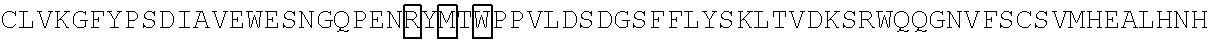

YTQKSLSLSPGK
SEQ ID NO: 115 (wildtype human PD-L2 extracellular domain-hinge-IgG1 Fe mutant2; PD-L2 extracellular
domain is underlined; hinge is bolded; linker is bolded and underlined)
LFTVTVPKELYIIEHGSNVTLECNFDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGDKTHTCPPCPA

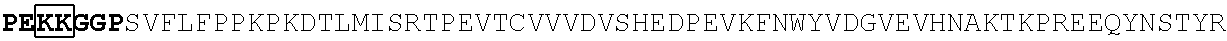

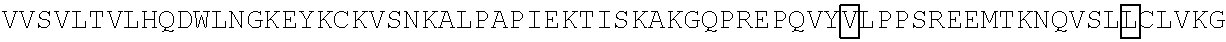

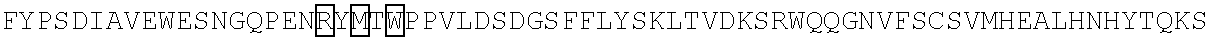

LSLSPGK
SEQ ID NO: 116 (human PD-L2 extracellular domain mutant2 S58V extracellular domain-linker-hinge-IgG1
Fc mutant1; PD-L2 extracellular domain is underlined; linker is bolded and underlined; hinge is bolded)

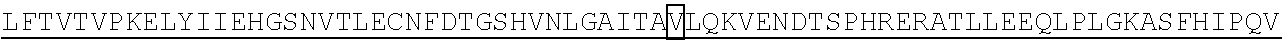

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNESCVFWNTHVRELTLASIDLQSQMEPRTHPTGGGGSGGGDKTHT

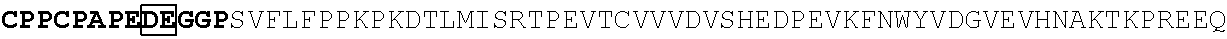

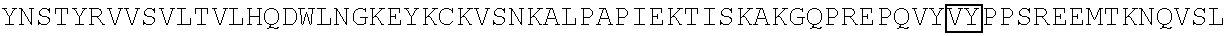

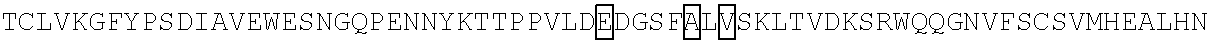

HYTQKSLSLSPGK
SEQ ID NO: 117 (buman PD-L2 extracellular domain mutant4 T56V/S58V/Q60L extracellular domain-
linker-hinge-IgG1 Fc mutant1; PD-L2 extracellular domain is underlined; linker is bolded and underlined;
hinge is bolded)

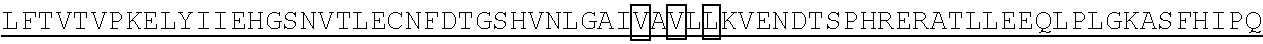

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGGSGGGDKTH

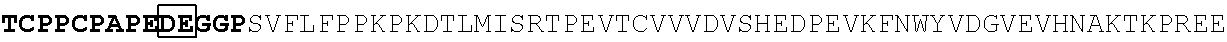

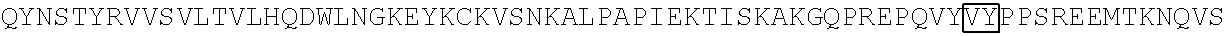

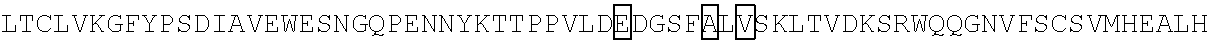

NHYTQKSLSLSPGK
SEQ ID NO: 118 (human PD-L2 extracellular domain mutant2 858V extracellular domain-linker-hinge-IgG1
Fc mutant2; PD-L2 extracellular domain is underlined; linker is bolded and underlined; hinge is bolded)

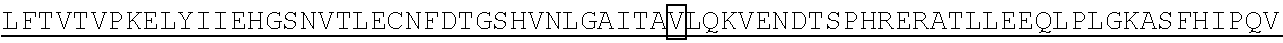

QVRDEGQYQCIIIYGVAWDYKYLTLKVKASYRKINTHILKVPETDEVELTCOATGYPLAEVSWPNVSVPAN
TSHSRTPEGLYQVTSVLRLKPPPGRNESCVFWNTHVRELTLASIDLQSQMEPRTHPTGGGGSGGGDKTHT

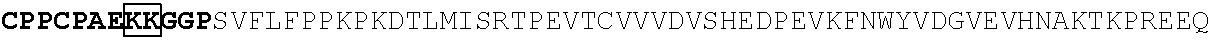

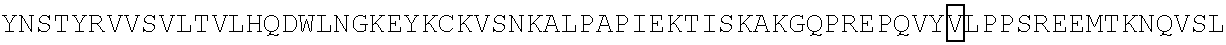

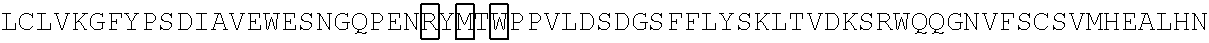

HYTQKSLSLSPGK
SEQ ID NO: 119 (human PD-L2 extracellular domain mutant4 TS6V/S58V/Q60L extracellular domain-
linker- hinge-IgG1 Fc mutant2; PD-L2 extracellular domain is underlined; linker is bolded and
underlined; hinge is bolded)

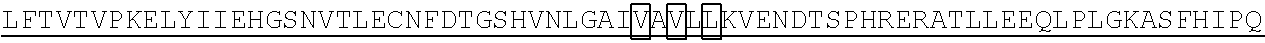

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLOSOMEPRTHPTGGGGSGGGDKTH

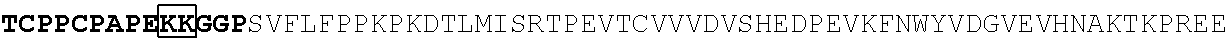

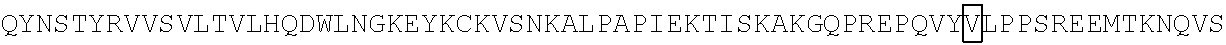

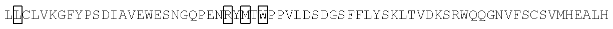

NHYTQKSLSLSPGK
SEQ ID NO: 120 (wildtype human PD-L1; signal peptide is italicized; extracellular domain is
underlined; cytoplasmic domain is bolded)
MRIFAVFIFMTYWHLLNAFTVTVPKDLYVVEYGSNMTIECKFPVEKOLDLAALIVYWEMEDKNIIQFVHGEE
DLKVQHSSYRQRARLLKDQLSLGNAALQITDVKLQDAGVYRCMISYGGADYKRITVKVNAPYNKINQRIL
VVDPVTSEHELTCQAEGYPKAEVIWTSSDHQVLSGKTTTTNSKREEKLFNVTSTLRINTTTNEIFYCTFRRL
DPEENHTAELVIPELPLAHPPNERTHLVILGAILLCLGVALTFIFRLRKGRMMDVKKCGIQDTNSKKQSDT
HLEET
SEQ ID NO: 121 (wildtype human PD-L1 extracellular domain)
FTVTVPKDLYVVEYGSNMTIECKFPVEKQLDLAALIVYWEMEDKNIIQFVHGEEDLKVQHSSYRQRARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFYCTFRRLDPEENHTAELVIPELPL
AHPPNER
SEQ ID NO: 122 (human PD-L1 extracellular domain mutant1 E58M/R113T/M115L/S117A/G119K)

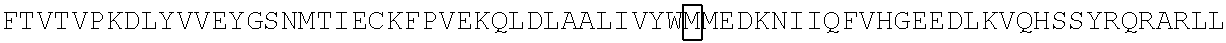

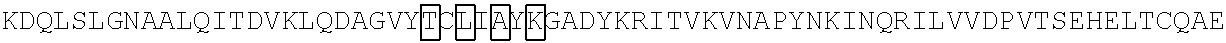

GYPKAEVIWTSSDHQVLSGKTTTTNSKREEKLFNVTSTLRINTTTNEIFYCTFRRLDPEENHTAELVIPELPL
AHPPNER
SEQ ID NO: 123 (human PD-L1 extracellular domain mutant2 154Q/E58M/R113T/M115L/S117A/G119K)

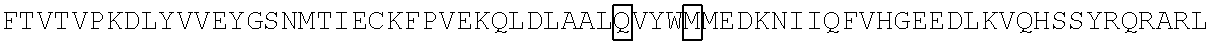

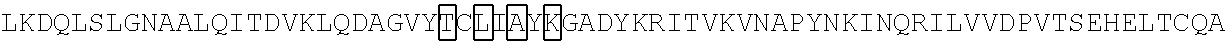

EGYPKAEVIWTSSDHQVLSGKTTTTNSKREEKLFNVTSTLRINTTTNEIFYCTFRRLDPEENHTAELVIPELPL
AHPPNER
SEQ ID NO: 124 (human PD-L1 extracellular domain mutant3 IS4Q/R113T/M115L/S117A/G119K)

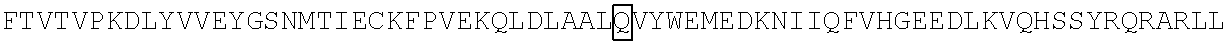

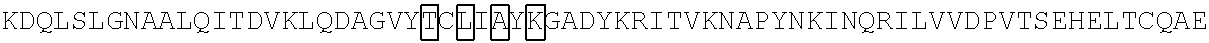

GYPKAEVIWTSSDHQVLSGKTTTTNSKREEKLFNVTSTLRINTTTNEIFYCTFRRLDPEENHTAEL VIPELPL
AHPPNER
SEQ ID NO: 125 (human PD-L1 extracellular domain mutant4 I54Q/E58M/M115L/S117A/G119K)

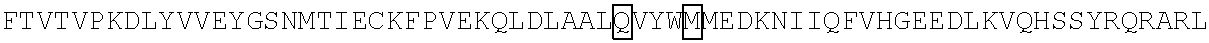

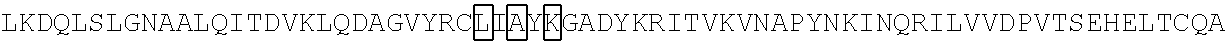

EGYPKAEVIWTSSDHQVLSGKTTTTNSKREEKLFNVTSTLRINTTTNEIFYCTFRRLDPEENHTAEL VIPELPL
AHPPNER
SEQ ID NO: 126 (human PD-L1 extracellular domain mutant5 IS4Q/E58M/R113T/S117A/G119K)

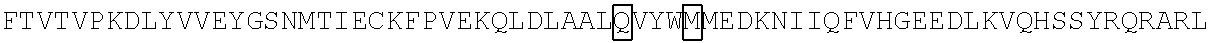

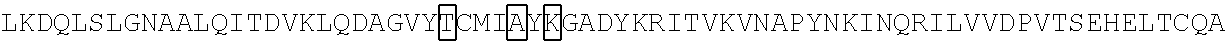

EGYPKAEVIWTSSDHQVLSGKTTTTNSKREEKLFNVTSTLRINTTTNEIFYCTFRRLDPEENHTAEL VIPELPL
AHPPNER
SEQ ID NO: 127 (human PD-L1 extracellular domain mutant6 154Q/E58M/R113T/M115L/G119K)

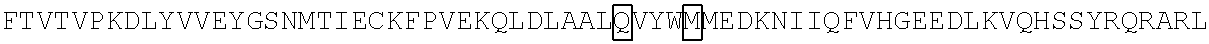

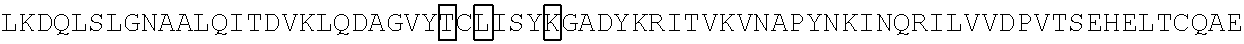

GYPKAEVIWTSSDHQVLSGKTTTTNSKREEKLFNVTSTLRINTTTNEIFYCTFRRLDPEENHTAELVIPELPL
AHPPNER
SEQ ID NO: 128 (human PD-L1 extracellular domain mutant7 154Q/E58M/R113T/M115L/S117A)

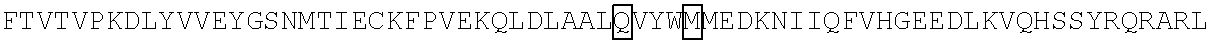

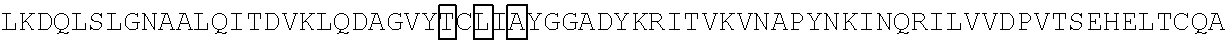

EGYPKAEVIWTSSDHQVLSGKTTTTNSKREEKLFNVTSTLRINTTTNEIFYCTFRRLDPEENHTAEL VIPELPL
AHPPNER
SEQ ID NO: 129 (human PD-L1 extracellular domain mutant8
154Q/Y56F/E58M/R113T/M115L/S117A/G119K)

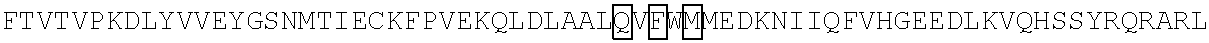

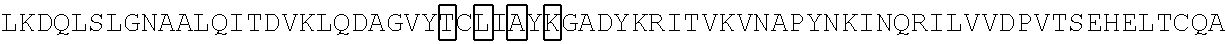

EGYPKAEVIWTSSDHQVLSGKTTTTNSKREEKLFNVTSTLRINTTTNEIFYCTFRRLDPEENHTAELVIPELPL
AHPPNER
SEQ ID NO: 130 (wildtype human PD-L1 extracellular domain-linker-hinge-IgG1 Fc mutant1; PD-L1
extracellular domain is underlined; linker is bolded and underlined; hinge is bolded)
FTVTVPKDLYVVEYGSNMTIECKFPVEKQLDLAALIVYWEMEDKNIIQFVHGEEDLKVQHSSYRQRARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFYCTFRRLDPEENHTAELVIPELPL

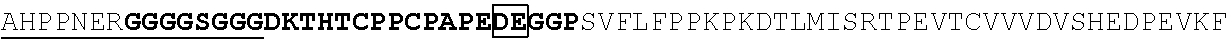

NWYVDGVEVHNAKTKPREEQYNSTYRVVSVLTVLHQDWLNGKEYKCKVSNKALPAPIEKTISKAKGQPR

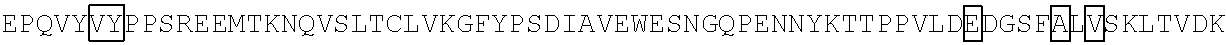

SRWQQGNVFSCSVMHEALHNHYTQKSLSLSPGK
SEQ ID NO: 131 (wildtype human PD-L1 extracellular domain-linker-hinge-IgG1 Fc mutant2; PD-L1
extracellular domain is underlined; linker is bolded and underlined; binge is bolded)
FTVTVPKDLYVVEYGSNMTIECKFPVEKQLDLAALIVYWEMEDKNIIQFVHGEEDLKVQHSSYRQRARLL
KDQLSLGNAALQITDVKLQDAGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFYCTFRRLDPEENHTAELVIPELPL

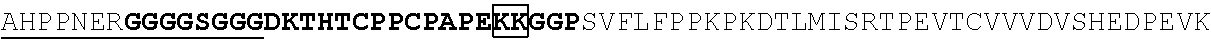

FNWYVDGVEVHNAKTKPREEQYNSTYRVVSVLTVLHQDWLNGKEYKCKVSNKALPAPIEKTISKAKGQP

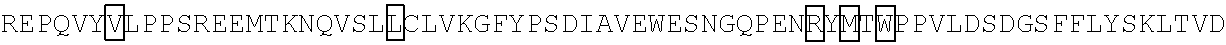

KSRWQQGNVFSCSVMHEALHNHYTQKSLSLSPGK
SEQ ID NO: 132 (human PD-L1 extracellular domain mutant2 I54Q/E58M/R113T/M115L/S117A/G119K
extracellular domain-linker-hinge-IgG1 Fc mutant1; PD-L1 extracellular domain is underlined; linker
is bolded and underlined; hinge is bolded)

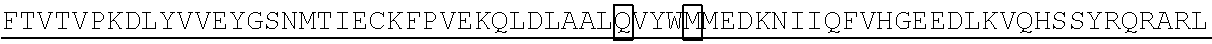

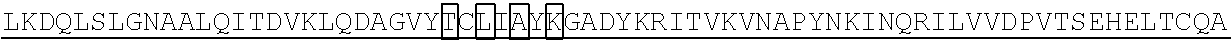

EGYPKAEVIWTSSDHQVLSGKTTTTNSKREEKLFNVTSTLRINTTTNEIFYCTFRRLDPEENHTAELVIPELPL

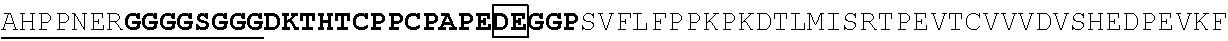

NWYVDGVEVHNAKTKPREEQYNSTYRVVSVLTVLHQDWLNGKEYKCKVSNKALPAPIEKTISKAKGQPR

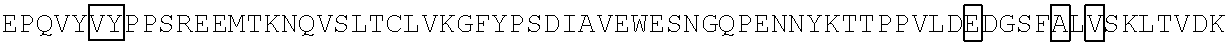

SRWQQGNVFSCSVMHEALHNHYTQKSLSLSPGK
SEQ ID NO: 133 (human PD-L1 extracellular domain matant7 I54Q/E58M/R113T/M115L/S117A
extracellular domain-linker-hinge-IgG1 Fc mutant1; PD-L1 extracellular domain is underlined; linker
is bolded and underlined; hinge is bolded)

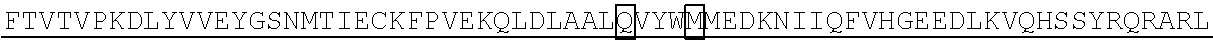

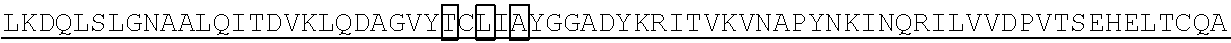

EGYPKAEVIWTSSDHQVLSGKTTTTNSKREEKLFNVTSTLRINTTTNEIFYCTFRRLDPEENHTAELVIPELPL

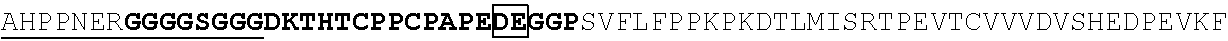

NWYVDGVEVHNAKTKPREEQYNSTYRVVSVLTVLHQDWLNGKEYKCKVSNKALPAPIEKTISKAKGQPR

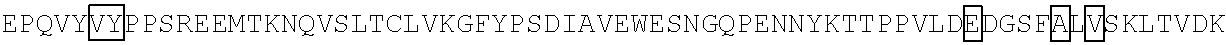

SRWQQGNVFSCSVMHEALHNHYTQKSLSLSPGK
SEQ ID NO: 134 (human PD-L1 extracellular domain mutant2 I54Q/E58M/R113T/M115L/S117A/G119K
extracellular domain-linker-hinge-IgG1 Fc mutant2; PD-L1 extracellular domain is underlined; linker
is bolded and underlined; hinge is bolded)

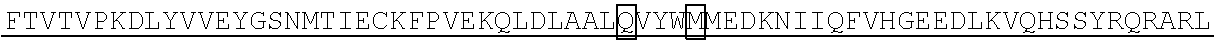

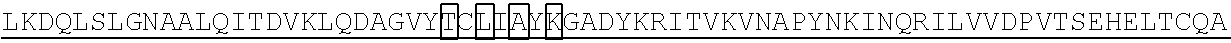

EGYPKAEVIWTSSDHQVLSGKTTTTNSKREEKLFNVTSTLRINTTTNEIFYCTFRRLDPEENHTAELVIPELPL

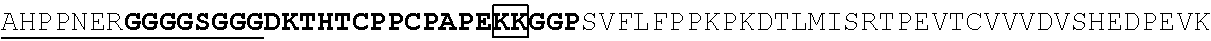

FNWYVDGVEVHNAKTKPREEQYNSTYRVVSVLTVLHQDWLNGKEYKCKVSNKALPAPIEKTISKAKGQP

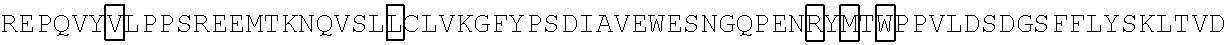

KSRWQQGNVFSCSVMHEALHNHYTQKSLSLSPGK
SEQ ID NO: 135 (human PD-L1 extracellular domain mutant7 I54Q/E58M/R113T/M115L/S117A
extracellular domain-linker- hinge-IgG1 Fc mutant2; PD-L1 extracellular domain is underlined; linker
is bolded and underlined; hinge is bolded)

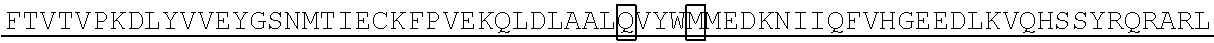

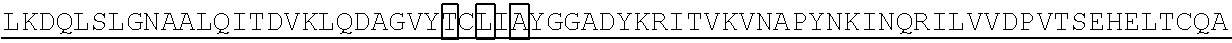

EGYPKAEVIWTSSDHQVLSGKTTTTNSKREEKLFNVTSTLRINTTTNEIFYCTFRRLDPEENHTAELVIPELPL

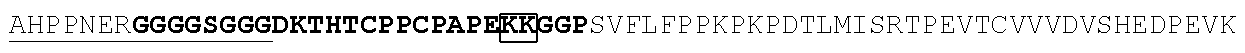

FNWYVDGVEVHNAKTKPREEQYNSTYRVVSVLTVLHQDWLNGKEYKCKVSNKALPAPIEKTISKAKGQP

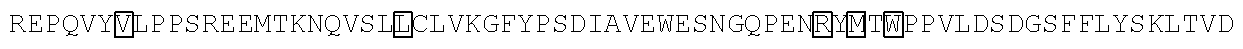

KSRWQQGNVFSCSVMHEALHNHYTQKSLSLSPGK
SEQ ID NO: 136 (Wild-type human CD155)
MARAMAAAWPLLLVALLVLSWPPPGTGDVVVQAPTQVPGFLGDSVTLPCYLQVPNMEVTHVSQLTWAR
HGESGSMAVFHQTQGPSYSESKRLEFVAARLGAELRNASLRMFGLRVEDEGNYTCLFVTFPQGSRSVDIWL
RVLAKPONTAEVQKVQLTGEPVPMARCVSTGGRPPAQITWHSDLGGMPNTSQVPGFLSGTVTVTSLWILV
PSSQVDGKNVTCKVEHESFEKPQLLTVNLTVYYPPEVSISGYDNNWYLGQNEATLTCDARSNPEPTGYNW
STTMGPLPPFAVAQGAQLLIRPVDKPINTTLICNVTNALGARQAELTVQVKEGPPSEHSGISRNAIIFLVLGIL
VFLILLGIGIYFYWSKCSREVLWHCHLCPSSTEHASASANGHVSYSAVSRENSSSQDPQTEGTR
SEQ ID NO: 137 (Wild-type human CD155 extracellular domain)
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPONTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP
VDKPINTTLICNVTNALGARQAELTVQVKEGPPSEHSGISRN
SEQ ID NO: 138 (human CD155 extracellular domain-linker-hinge-IgG1 Fc mutant1; CD155 extracellular
domain is underlined; linker is bolded and underlined; hinge is bolded)
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSOVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

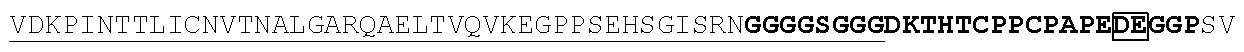

FLFPPKPKDTLMISRTPEVTCVVVDVSHEDPEVKFNWYVDGVEVHNAKTKPREEQYNSTYRVVSVLTVLH

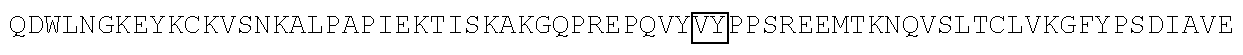

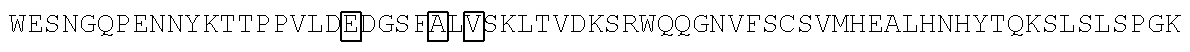

SEQ ID NO: 139 (human CD155 extracellular domain-linker-hinge-IgG1 Fc mutant2; CD155 extracellular
domain is underlined; linker is bolded and underlined; hinge is bolded)
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGELSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

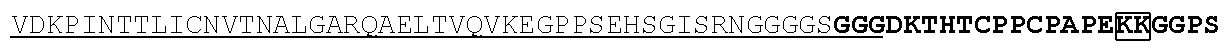

VFLFPPKPKDTLMISRTPEVTCVVVDVSHEDPEVKFNWYVDGVEVHNAKTKPREEQYNSTYRVVSVLTVL

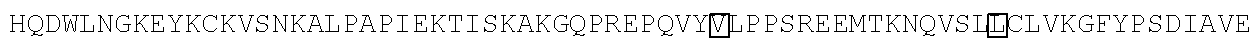

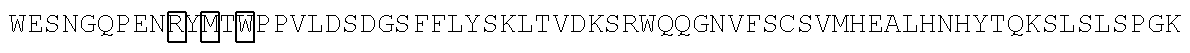

SEQ ID NO: 140 (IL-12B (p40) mutant F60D subunit)

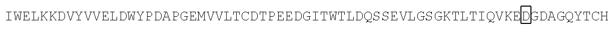

KGGEVLSHSLLLLHKKEDGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVHKLKYENYTSSFFIRDIIKP
DPPKNLQLKPLKNSRQVEVSWEYPDTWSTPHSYFSLTFCVQVQGKSKREKKDRVFTDKTSATVICRKNASI
SVRAQDRYYSSSWSEWASVPCS
SEQ ID NO: 141 (wildtype human CTLA-4 extracellular domain)
KAMHVAQPAVVLASSRGIASFVCEYASPGKATEVRVTVLRQADSQVTEVCAATYMMGNELTFLDDSICTG
TSSGNQVNLTIQGLRAMDTGLYICKVELMYPPPYYLGIGNGTQIYVIDPEPCPDSD
SEQ ID NO: 142 (human PD-L2 extracellular domain hinge portion-linker-single-chain IL-12 mutant
heterodimer IL-12B (p40 F60A)-linker IL-12A (wt p35)-C' hinge portion-IgG1 Fc mutant1; VH is
underlined; hinge is bolded; linker is bolded and underlined; IL-12 subunits are italicized)
LFTVTVPKELYIIEHGSNVTLECNEDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGIWELKKDVYVVEL

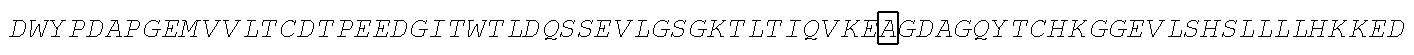

GIWSTDILKDQKEPKNKTFLRCEAKNYSGRFTCWWLTTISTDLTFSVKSSRGSSDPQGVTCGAATLSAERVRGDNK
EYEYSVECQEDSACPAAEESLPIEVMVDAVHKLKYENYTSSFFIRDIIKPDPPKNLQLKPLKNSROVEVSWEYPDTW
STPHSYFSLTFCVQVQGKSKREKKDRVFTDKTSATVICRKNASISVRAQDRYYSSSWSEWASVPCSGGGGSGGGG
SGGGGSGGGGSGRNLPVATPDPGMFPCLHHSQNLLRAVSNMLQKAROTLEFYPCTSEEIDHEDITKDKTSTVE
ACLPLELTKNESCLNSRETSFITNGSCLASRKTSEMMALCLSSIYEDLKMYQVEFKTMNAKLLMDPKRQIFLDQNM

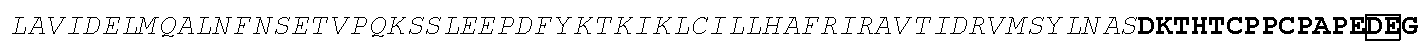

GPSVFLFPPKPKDTLMISRTPEVTCVVVDVSHEDPEVKFNWYVDGVEVHNAKTKPREEQYNSTYRVVSVL

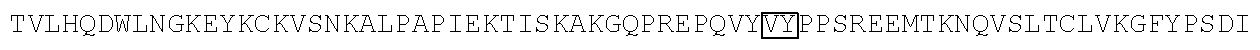

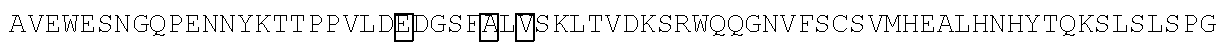

K
SEQ ID NO: 143 (human PD-L2 extracellular domain-linker-hinge portion-IgG1 Fe mutant1-linker-single-
chain IL-12 mutant heterodimer IL-12B (p40 F60A)-linker IL-12A (wt p35); VH is underlined; hinge is
bolded; linker is bolded and underlined; IL-12 subunits are italicized)
LFTVTVPKELYIIEHGSNVTLECNEDTGSHVNLGAITASLQKVENDTSPHRERATLLEEQLPLGKASFHIPQV
QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGSGDKTHTCPPCPA

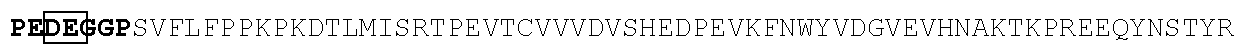

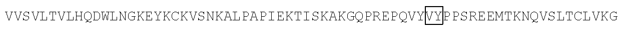

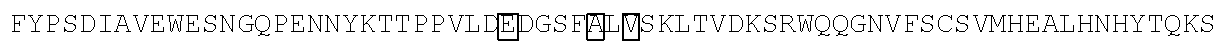

LSLSPGKGGGGSGGGGSGGGGSIWELKKDVYVVELDWYPDAPGEMVVLTCDTPEEDGITWTLDQSSEVLGSG

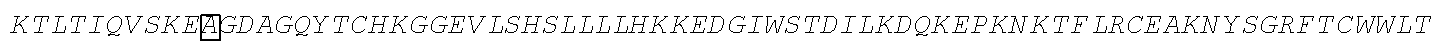

TISTDLTFSVKSSRGSSDPQGVTCGAATLSAERVRGDNKEYEYSVECQEDSACPAAEESLPIEVMVDAVHKLKYENY
TSSFFIRDIIKPDPPKNLOLKPLKNSROVEVSWEYPDTWSTPHSYFSLTFCVQVQGKSKREKKDRVFTDKTSATVIC
RKNASISVRAQDRYYSSSWSEWASVPCSGGGGSGGGGSGGGGSGGGGSGRNLPVATPDPGMFPCLHHSQNLL
RAVSNMLQKARQTLEFYPCTSEEIDHEDITKDKTSTVEACLPLELTKNESCLNSRETSFITNGSCLASRKTSFMMAL
CLSSIYEDLKMYQVEFKTMNAKLLMDPKRQIFLDQNMLAVIDELMQALNENSETVPQKSSLEEPDFYKTKIKLCIL
LHAFRIRAVTIDRVMSYLNAS
SEQ ID NO: 144 (anti-PD-1 Ab VH-CHI-N' hinge portion-IL-2 mutant R38D/K43E/E61R-C' hinge portion-
IgG1 Fc mutant1; VH is underlined; hinge is bolded; IL-2 mutant is italicized)
QVQLVESGGGVVOPGRSLRLDCKASGITESNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

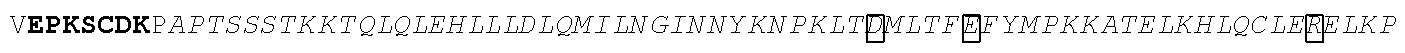

LEEVLNLAQSKNFHLRPRDLISNINVIVLELKGSETTEMCEYADETATIVEFLNRWITFCOSIISTLTDKTHTCPPCP

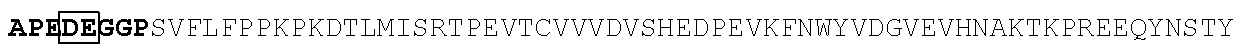

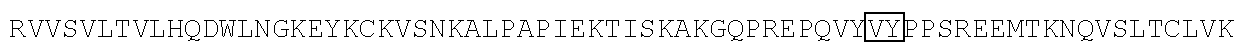

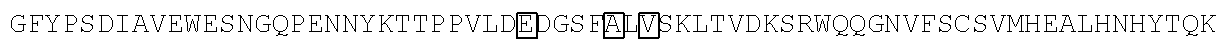

SLSLSPGK
SEQ ID NO: 145 (anti-PD-1 Ab VH-CH1-N' hinge portion-linker-single-chain IL-23 mutant heterodimer IL-
12B (p40 E59A/F60A)-linker-IL-23A (wt p19)-C' hinge portion-IgG1 Fc mutant1; VH is underlined; hinge
is bolded; linker is bolded and underlined; IL-23 subunits are italicized)
QVQLVESGGGVVQPGRSLRLDCKASGITESNSGMHWVRQAPGKGLEWVAVIWYDGSKRYYADSVKGREF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

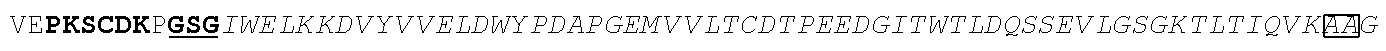

DAGQYTCHKGGEVLSHSLLLLHKKEDGIWSTDILKDQKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRAVPGGSSPAWTQCQQLSOKLCTLAWSAHPLVGH
MDLREEGDEETTNDVPHIQCGDGCDPQGLRDNSQFCLQRIHQGLIFYEKLLGSDIFTGEPSLLPDSPVGQLHASL
LGLSQLLQPEGHHWETQQIPSLSPSQPWQRLLLRFKILRSLOAFVAVAARVFAHGAATLSPDKTHTCPPCPAPE

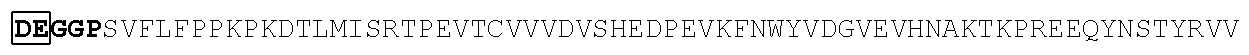

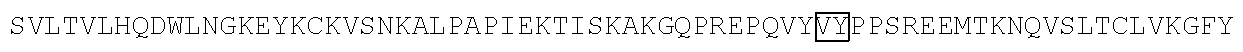

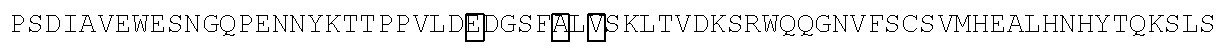

LSPGK
SEQ ID NO: 146 (anti-PD-1 Ab VH-CHI-N' hinge portion-linker-single-chain IL-10 mutant R27A
homodimer-C' hinge portion-IgG1 Fc mutant1; VH is underlined; hinge is bolded; linker is bolded and
underlined; IL-10 mutant monomer is italicized)
QVQLVESGGGVVQPGRSLRLDCKASGITESNSGMHWVRQAPGKGLEWVAVIWYDGSKRYYADSVKGRF
TISRDNSKNTLFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

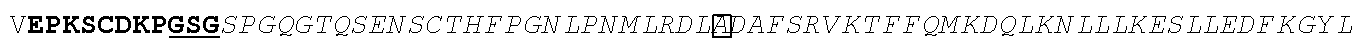

GCQALSEMIQFYLEEVMPOAENQDPDIKAHVNSLGENLKTLRLRLRRCHRFLPCENKSKAVEQVKNAFNKLQEK
GIYKAMSEFDIFINYIEAYMTMKIRNFEGGGSGGGGSGGGGSGGGGSSPGQGTQSENSCTHFPGNLPNMLRD

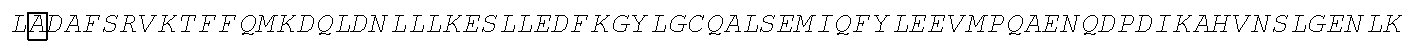

TLRLRLRRCHRFLPCENKSKAVEQVKNAFNKLQEKGIYKAMSEFDIFINYIEAYMTMKIRNDKTHTCPPCPAPE

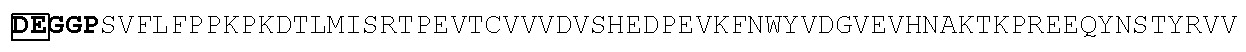

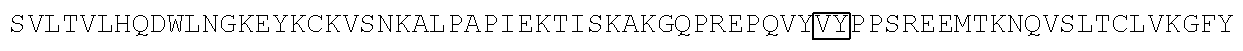

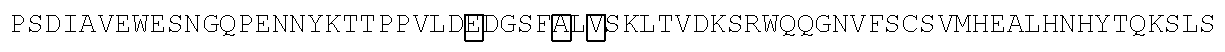

LSPGK
SEQ ID NO: 147 (anti-PD-1 Ab VH-CHI-N' hinge portion-linker-single-chain IFN-γ mutant A23V
bomodimer-C' hinge portion-IgG1 Fc mutant1; VH is underlined; hinge is bolded; linker is bolded and
underlined; IFN-γ mutant monomer is italicized)
QVQLVESGGGVVQPGRSLRLDCKASGITFSNSGMHWVRQAPGKGLEWVAVIWYDGSKRYYADSVKGRF
TISRDNSKNILFLQMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

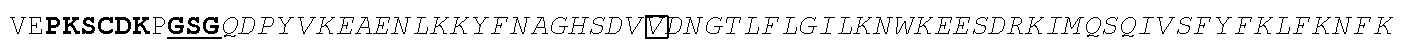

DDQSIQKSVETIKEDMNVKFFNSNKKKRDDFEKLINYSVTDLNVQRKAIHELIQVMAELSPAAKTGKRKRSQMLF

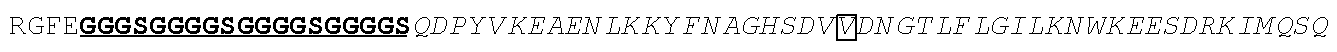

IVSFYFKLEKNFKDDQSIQKSVETIKEDMNVKFFNSNKKKRDDFEKLINYSVTDLNVQRKAIHELIQVMAELSPAA

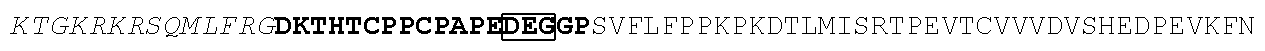

WYVDGVEVHNAKTKPREEQYNSTYRVVSVLTVLHQDWLNGKEYKCKVSNKALPAPIEKTISKAKGQPRE

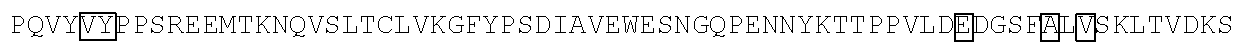

RWQQGNVFSCSVMHEALHNHYTQKSLSLSPGK
SEQ ID NO: 148 (anti-PD-1 Ab VH-CHI-N' hinge portion-linker-IFN-a2b matant L30A-C' hinge portion-
IgG1 Fc mutant1; VH is underlined; hinge is bolded; linker is bolded and underlined; IFN-a2b mutant is
italicized)
QVQLVESGGGVVQPGRSLRLDCKASGITFSNSGMHWVRQAPGKGLEWVAVIWYDGSKRYYADSVKGRF
TISRDNSKNTLFLOMNSLRAEDTAVYYCATNDDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

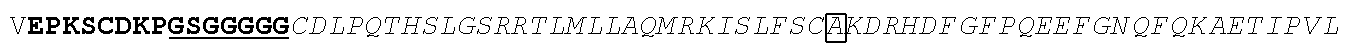

HEMIQQIFNLFSTKDSSAAWDETLLDKFYTELYQQLNDLEACVIQGVGVTETPLMKEDSILAVRKYFQRITLYLKE

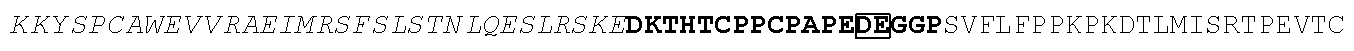

VVVDVSHEDPEVKFNWYVDGVEVHNAKTKPREEQYNSTYRVVSVLTVLHQDWLNGKEYKCKVSNKALP

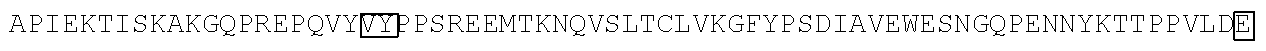

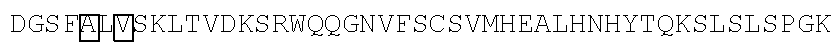

SEQ ID NO: 149 (anti-PD-1 Ab VH(D100N)-CH1-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
QVQLVESGGGVVOPGRSLRLDCKASGITFSNSGMHWVRQAPGKGLEWVAVIWYDGSKRYYADSVKGRF

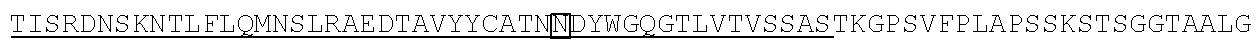

CLVKDYFPEPVTVSWNSGALTSGVHTFPAVLQSSGLYSLSSVVTVPSSSLGTQTYICNVNHKPSNTKVDKK

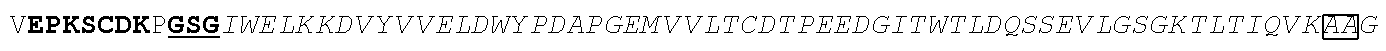

DAGQYTCHKGGEVLSHSLLLLHKKEDGIWSTDILKDQKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSROVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSQNLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNENSETVPQKSSLEEPDFYKTKIKLCILLHAFRIRAVTID

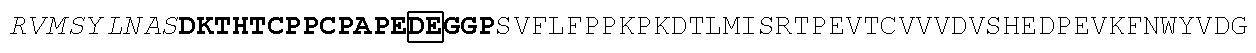

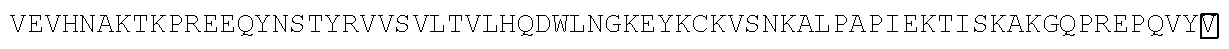

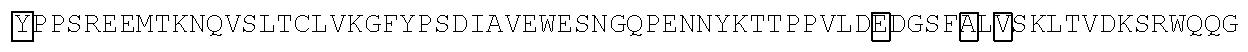

NVFSCSVMHEALHNHYTQKSLSLSPGK
SEQ ID NO: 150 (anti-PD-1 Ab VH(D100G)-CHI-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
QVQLVESGGGVVQPGRSLRLDCKASGITFSNSGMHWVRQAPGKGLEWVAVIWYDGSKRYYADSVKGRF

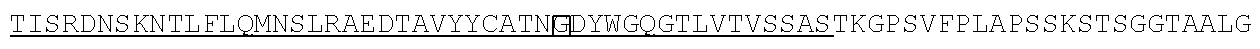

CLVKDYFPEPVTVSWNSGALTSGVHTFPAVLQSSGLYSLSSVVTVPSSSLGTQTYICNVNHKPSNTKVDKK

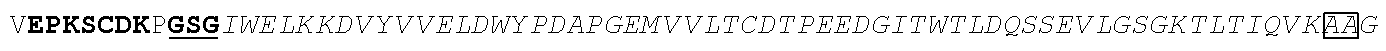

DAGQYTCHKGGEVLSHSLLLLHKKEDGIWSTDILKDQKEPKNKTFLRCEAKNYSGRFTCWWLTTISTDLTESVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSQNLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNENSETVPQKSSLEEPDFYKTKIKLCILLHAFRIRAVTID

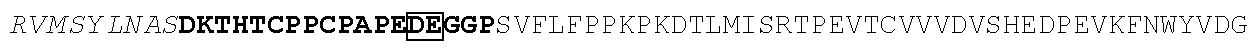

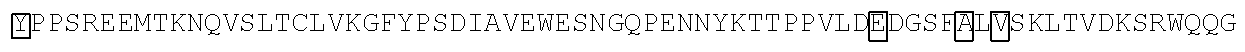

NVFSCSVMHEALHNHYTQKSLSLSPGK
SEQ ID NO: 151 (anti-PD-1 Ab VH(D100R)-CHI-N' hinge portion-linker-single-chain IL-12 mutant
heterodimer IL-12B (p40 E59A/F60A)-linker IL-12A (wt p35)-C' hinge portion-IgG1 Fe mutant1; VH is
underlined; hinge is bolded; linker is bolded and underlined; IL-12 subunits are italicized)
QVQLVESGGGVVOPGRSLRLDCKASGITFSNSGMHWVRQAPGKGLEWVAVIWYDGSKRYYADSVKGRF

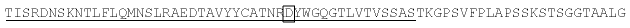

CLVKDYFPEPVTVSWNSGALTSGVHTFPAVLQSSGLYSLSSVVTVPSSSLGTQTYICNVNHKPSNTKVDKK

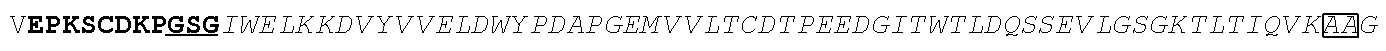

DAGQYTCHKGGEVLSHSLLLLHKKEDGIWSTDILKDQKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSQNLLRAVSNMLQKAR
QTLEFYPCTSEEIDHEDITKDKTSTVEACLPLELTKNESCLNSRETSFITNGSCLASRKTSEMMALCLSSIYEDLKMY
QVEFKTMNAKLLMDPKRQIFLDQNMLAVIDELMQALNENSETVPQKSSLEEPDFYKTKIKLCILLHAFRIRAVTID

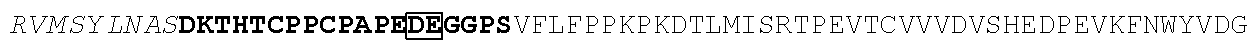

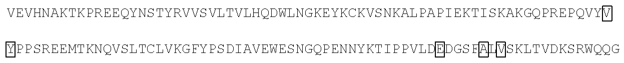

NVFSCSVMHEALHNHYTQKSLSLSPGK
SEQ ID NO: 152 (anti-PD-1 Ab VH(N99G)-CHI-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
QVQLVESGGGVVQPGRSLRLDCKASGITFSNSGMHWVRQAPGKGLEWVAVIWYDGSKRYYADSVKGRF

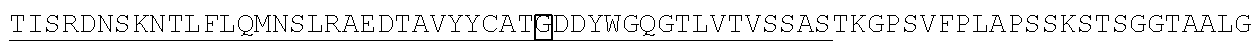

CLVKDYFPEPVTVSWNSGALTSGVHTFPAVLQSSGLYSLSSVVTVPSSSLGTQTYICNVNHKPSNTKVDKK

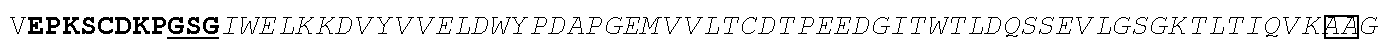

DAGQYTCHKGGEVLSHSLLLLHKKEDGIWSTDILKDQKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGGGGGSGRNLPVATPDPGMFPCLHHSONLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNENSETVPQKSSLEEPDFYKTKIKLCILLHAFRIRAVTID

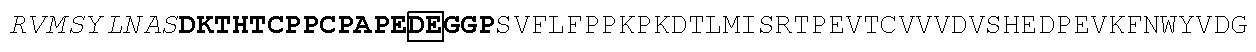

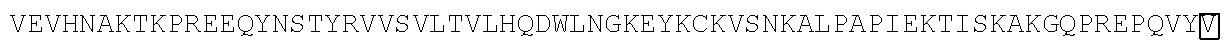

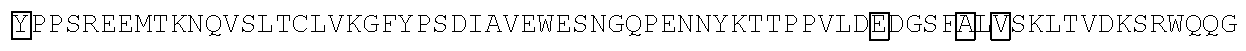

NVFSCSVMHEALHNHYTQKSLSLSPGK
SEQ ID NO: 153 (anti-PD-1 Ab VH(N99A)-CH1-N' hinge portion-linker-single-chain IL-12 mutant
heterodimer IL-12B (p40 E59A/F60A)-linker IL-12A (wt p35)-C' hinge portion-IgG1 Fc mutant1; VH is
underlined; hinge is bolded; linker is bolded and underlined; IL-12 subunits are italicized)
QVQLVESGGGVVOPGRSLRLDCKASGITFSNSGMHWVRQAPGKGLEWVAVIWYDGSKRYYADSVKGRF

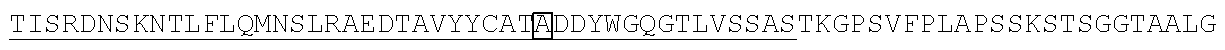

CLVKDYFPEPVTVSWNSGALTSGVHTFPAVLQSSGLYSLSSVVTVPSSSLGTQTYICNVNHKPSNTKVDKK

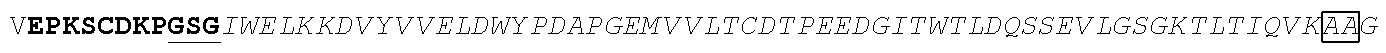

DAGQYTCHKGGEVLSHSLLLLHKKEDGIWSTDILKDOKEPKNKTFLRCEAKNYSGRFTCWWLTTISTDLIFSVKSS
RGSSDPQGVTCGAATLSAERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKTSATVICRKNASISVRAQD
RYYSSSWSEWASVPCSGGGGSGGGGSGGGGSGGGGSGRNLPVATPDPGMFPCLHHSQNLLRAVSNMLQKAR
QTLEFYPCTSEEIDHEDITKDKTSTVEACLPLELTKNESCLNSRETSFITNGSCLASRKTSFMMALCLSSIYEDLKMY
QVEFKTMNAKLLMDPKRQIFLDQNMLAVIDELMQALNFNSETVPQKSSLEEPDFYKTKIKLCILLHAFRIRAVTID

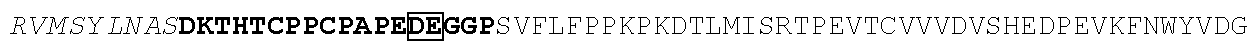

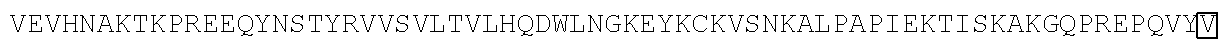

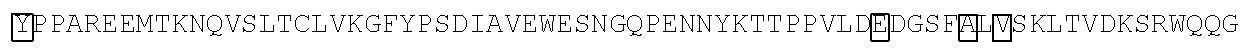

NVFSCSVMHEALHNHYTQKSLSLSPGK
SEQ ID NO: 154 (anti-PD-1 Ab VH(N99M)-CHI-N' hinge portion-linker-single-chain IL-12 mutant
heterodimer IL-12B (p40 ES9A/F60A)-linker IL~12A (wt p35)-C' hinge portion-IgG1 Fc mutant1; VH is
underlined; hinge is bolded; linker is bolded and underlined; IL-12 subunits are italicized)
QVQLVESGGGVVQPGRSLRLDCKASGITFSNSGMHWVRQAPGKGLEWVAVIWYDGSKRYYADSVKGRF

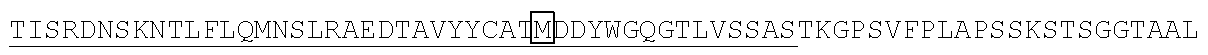

GCLVKDYFPEPVTVSWNSGALTSGVHTFPAVLOSSGLYSLSSVVTVPSSSLGTQTYICNVNHKPSNTKVDK

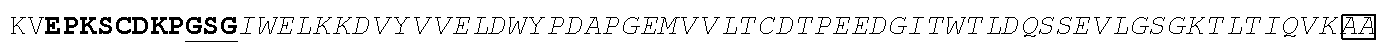

GDAGQYTCHKGGEVLSHSLLLLHKKEDGIWSTDILKDOKEPKNKTFLRCEAKNYSGRFTCWWLTTISTDLTFSVK
SSRGSSDPQGVTCGAATLSAERVRGDNKEYEYSVECQEDSACPAAEESLPIEVMVDAVHKLKYENYTSSFFIRDIIK
PDPPKNLQLKPLKNSROVEVSWEYPDTWSTPHSYFSLTFCVQVQGKSKREKKDRVFTDKTSATVICRKNASISVRA
QDRYYSSSWSEWASVPCSGGGGSGGGGSGGGGSGGGGSGRNLPVATPDPGMFPCLHHSQNLLRAVSNMLQK
ARQTLEFYPCTSEEIDHEDITKDKTSTVEACLPLELTKNESCLNSRETSFITNGSCLASRKTSFMMALCLSSIYEDLK
MYQVEFKIMNAKLLMDPKRQIFLDQNMLAVIDELMQALNENSETVPOKSSLEEPDFYKTKIKLCILLHAFRIRAVT

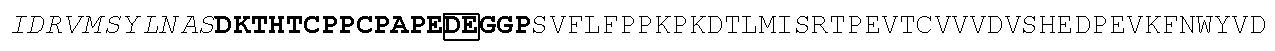

GVEVHNAKTKPREEQYNSTYRVVSVLTVLHQDWLNGKEYKCKVSNKALPAPIEKTISKAKGQPREPQVY

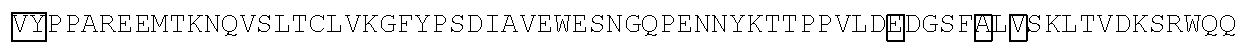

GNVFSCSVMHEALHNHYTQKSLSLSPGK
SEQ ID NO: 155 (haman PD-L1 extracellular domain matant2 I54Q/E58M/R113T/M115L/S117A/G119K
extracellular domain-linker-single-chain IL-12 mutant heterodimer IL-12B (p40 E59A/F60A)-linker IL~12A
(wt p35)-hinge-IgG1 Fc mutant1; PD-L1 extracellular domain is underlined; linker is bolded and
underlined; hinge is bolded; IL-12 subunits are italicized)

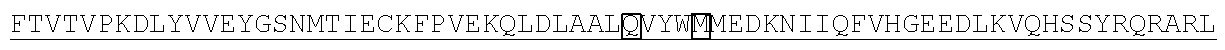

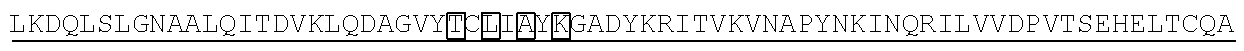

EGYPKAEVIWTSSDHQVLSGKTTTTNSKREEKLENVTSTLRINTTTNEIFYCTFRRLDPEENHTAELVIPELPL
AHPPNERGGGGSGGGIWELKKDVYVVELDWYPDAPGEMVVLTCDTPEEDGITWTLDQSSEVLGSGKTLTIQVK

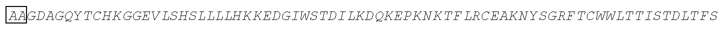

VKSSRGSSDPQGVTCGAATLSAERVRGDNKEYEYSVECQEDSACPAAEESLPIEVMVDAVHKLKYENYTSSFFIRDI
IKPDPPKNLOLKPLKNSRQVEVSWEYPDTWSTPHSYFSLTFCVOVOGKSKREKKDRVFTDKTSATVICRKNASISV
RAQDRYYSSSWSEWASVPCSGGGGSGGGSGGGGSRNLPVATPDPGMFPCLHHSQNLLRAVSNMLQKARQTLE
FYPCTSEEIDHEDITKDKISTVEACLPLELTKNESCLNSRETSFITNGSCLASRKTSEMMALCLSSIYEDLKMYQVEF
KTMNAKLLMDPKRQIFLDQNMLAVIDELMQALNENSETVPQKSSLEEPDFYKTKIKLCILLHAFRIRAVTIDRVMS

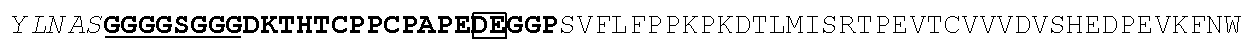

YVDGVEVHNAKTKPREEQYNSTYRVVSVLTVLHQDWLNGKEYKCKVSNKALPAPIEKTISKAKGQPREPQ

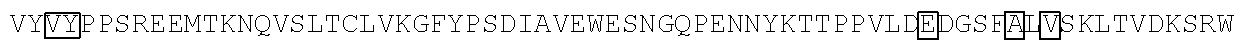

QQGNVFSCSVMHEALHNHYTQKSLSLSPGK
SEQ ID NO: 156 (human PD-L1 extracellular domain mutant7 I54Q/E58M/R113T/M115L/S117A
extracellular domain-linker-single-chain IL-12 mutant heterodimer IL-12B (p40 E59A/F60A)-linker IL-12A
(wt p35)-hinge-IgG1 Fc mutant1; PD-L1 extracellular domain is underlined; linker is bolded and
underlined; hinge is bolded; IL-12 subunits are italicized)

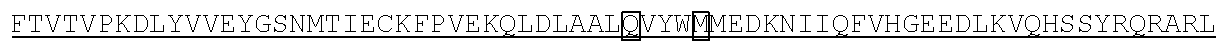

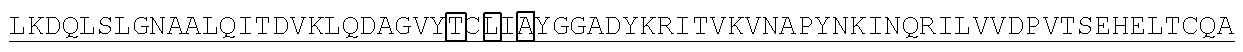

EGYPKAEVIWTSSDHQVLSGKTTTTNSKREEKLFNVTSTLRINTTTNEIFYCTFRRLDPEENHTAELVIPELPL
AHPPNERGGGGSGGGIWELKKDVYVVELDWYPDAPGEMVVLTCDTPEEDGITWTLDOSSEVLGSGKTLTIQVK

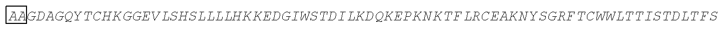

VKSSRGSSDPQGVTCGAATLSAERVRGDNKEYEYSVECQEDSACPAAEESLPIEVMVDAVHKLKYENYTSSFFIRDI
IKPDPPKNLQLKPLKNSRQVEVSWEYPDTWSTPHSYFSLTFCVQVQGKSKREKKDRVFTDKTSATVICRKNASISV
RAQDRYYSSSWSEWASVPCSGGGGSGGGSGGGGSRNLPVATPDPGMFPCLHHSQNLLRAVSNMLQKARQTLE
FYPCTSEEIDHEDITKDKTSTVEACLPLELTKNESCLNSRETSFITNGSCLASRKTSFMMALCLSSIYEDLKMYOVEF
KTMNAKLLMDPKRQIFLDQNMLAVIDELMQALNFNSETVPQKSSLEEPDFYKTKIKLCILLHAFRIRAVTIDRVMS

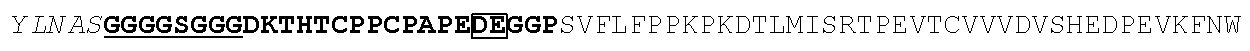

YVDGVEVHNAKTKPREEQYNSTYRVVSVLTVLHQDWLNGKEYKCKVSNKALPAPIEKTISKAKGQPREPQ

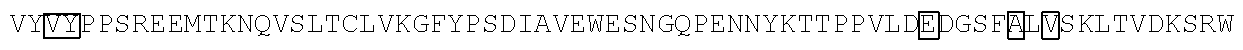

QQGNVFSCSVMHEALHNHYTQKSLSLSPGK
SEQ ID NO: 157 (buman PD-L1 extracellular domain mutant2 I54Q/E58M/R113T/M115L/S117A/G119K
extracellular domain-linker-hinge-lgG1 Fc mutant1-linker-single-chain IL-12 mutant heterodimer
IL~12B(p40 E59A/F60A)-linker IL-12A (wt p35); PD-L1 extracellular domain is underlined; linker is
bolded and underlined; hinge is bolded; IL-12 subunits are italicized)

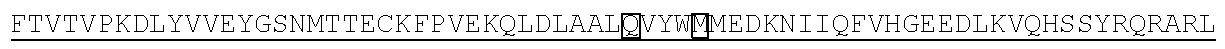

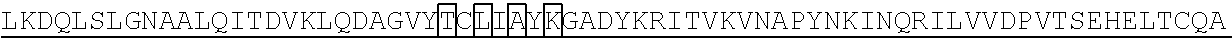

EGYPKAEVIWTSSDHQVLSGKTTTTNSKREEKLFNVTSTLRINTTTNEIFYCTFRRLDPEENHTAELVIPELPL

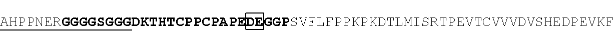

NWYVDGVEVHNAKTKPREEQYNSTYRVVSVLTVLHQDWLNGKEYKCKVSNKALPAPIEKTISKAKGQPR

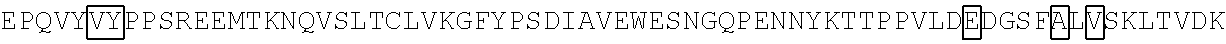

SRWQQGNVFSCSVMHEALHNHYTQKSLSLSPGKGGGGSGGGIWELKKDVYVVELDWYPDAPGEMVVLTCD

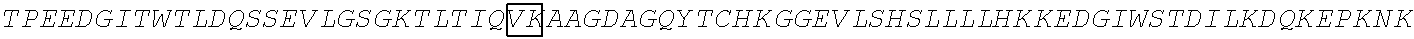

TFLRCEAKNYSGRFTCWWLTTISTDLTFSVKSSRGSSDPQGVTCGAATLSAERVRGDNKEYEYSVECQEDSACPAA
EESLPIEVMVDAVHKLKYENYTSSFFIRDIIKPDPPKNLQLKPLKNSRQVEVSWEYPDTWSTPHSYFSLTFCVOVOG
KSKREKKDRVFTDKTSATVICRKNASISVRAQDRYYSSSWSEWASVPCSGGGGSGGGSGGGGSRNLPVATPDPG
MFPCLHHSQNLLRAVSNMLQKAROTLEFYPCTSEEIDHEDITKDKTSTVEACLPLELTKNESCLNSRETSFITNGSC
LASRKTSEMMALCLSSIYEDLKMYQVEFKTMNAKLLMDPKRQIFLDQNMLAVIDELMQALNENSETVPQKSSLEE
PDFYKTKIKLCILLHAFRIRAVTIDRVMSYLNAS
SEQ ID NO: 158 (human PD-L1 extracellular domain mutant7 I54Q/E58M/R113T/M115L/S117A
extracellular domain-linker-binge-IgG1 Fc mutant1-linker-single-chain IL-12 mutant heterodimer
IL~12B(p40 E59A/F60A)-linker IL-12A (wt p35); PD-L1 extracellular domain is underlined; linker is
bolded and underlined; hinge is bolded; IL-12 subunits are italicized)

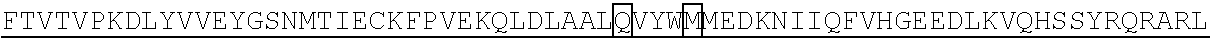

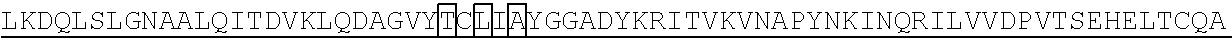

EGYPKAEVIWTSSDHQVLSGKTTTTNSKREEKLFNVTSTLRINTTTNEIFYCTERRLDPEENHTAELVIPELPL

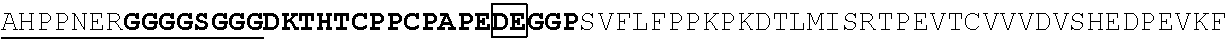

NWYVDGVEVHNAKTKPREEQYNSTYRVVSVLTVLHQDWLNGKEYKCKVSNKALPAPIEKTISKAKGQPR

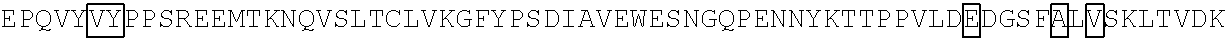

SRWQQGNVFSCSVMHEALHNHYTOKSLSLSPGKGGGGSGGGIWELKKDVYVVELDWYPDAPGEMVVLTCD

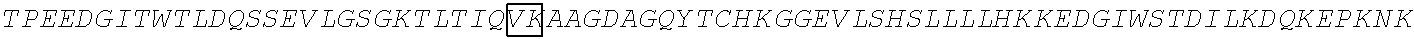

TFLRCEAKNYSGRFTCWWLTTISTDLTFSVKSSRGSSDPQGVTCGAATLSAERVRGDNKEYEYSVECOEDSACPAA
EESLPIEVMVDAVHKLKYENYTSSFFIRDIIKPDPPKNLQLKPLKNSRQVEVSWEYPDTWSTPHSYFSLTFCVOVOG
KSKREKKDRVFTDKTSATVICRKNASISVRAQDRYYSSSWSEWASVPCSGGGGSGGGSGGGGSRNLPVATPDPG
MFPCLHHSQNLLRAVSNMLOKAROTLEFYPCTSEEIDHEDITKDKTSTVEACLPLELTKNESCLNSRETSFITNGSC
LASRKTSFMMALCLSSIYEDLKMYQVEFKTMNAKLLMDPKRQIFLDQNMLAVIDELMQALNFNSETVPQKSSLEE
PDFYKTKIKLCILLHAFRIRAVTIDRVMSYLNAS
SEQ ID NO: 159 (human PD-L1 extracellular domain mutant2 I54Q/E58M/R113T/M115L/S117A/G119K
extracellular domain-linker-IL-2 mutant (L18R/Q22E/R38D/K43E/E61R)-linker-hinge-IgG1 Fc mutant2;
PD-L1 extracellular domain is underlined; linker is bolded and underlined; hinge is bolded; IL-12
subunits are italicized)

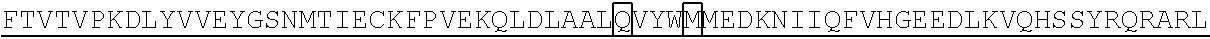

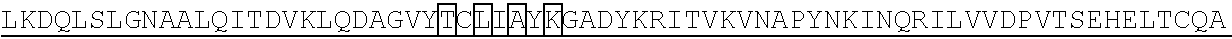

EGYPKAEVIWTSSDHQVLSGKTTTTNSKREEKLFNVTSTLRINTTTNEIFYCTFRRLDPEENHTAELVIPELPL

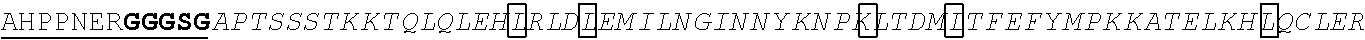

ELKPLEEVLNLAQSKNFHLRPRDLISNINVIVLELKGSETTFMCEYADETATIVEFLNRWITFCQSIISTLT

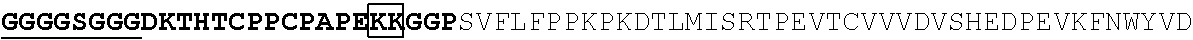

GVEVHNAKTKPREEQYNSTYRVVSVLTVLHQDWLNGKEYKCKVSNKALPAPIEKTISKAKGQPREPQVY

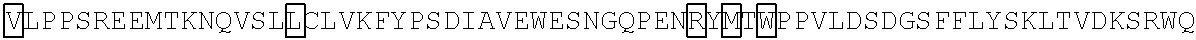

QGNVFSCSVMHEALHNHYTOKSLSLSPGK
SEQ ID NO: 160 (human PD-L1 extracellular domain mutant2 I54Q/E58M/R113T/M115L/S117A/G119K
extracellular domain-linker-IL-2 mutant (R38D/K43E/E61R/Q126T)-linker-hinge-IgG1 Fc mutant2; PD-L1
extracellular domain is underlined; linker is bolded and underlined; binge is bolded; IL-12 subunits
are italicized)

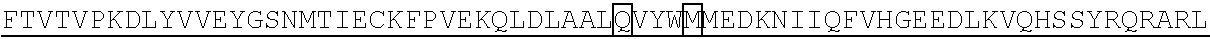

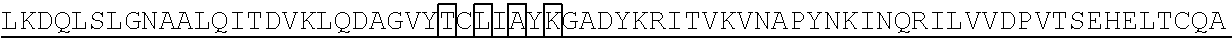

EGYPKAEVIWTSSDHOVLSGKTTTTNSKREEKLFNVTSTLRINTTTNEIFYCTFRRLDPEENHTAELVIPELPL

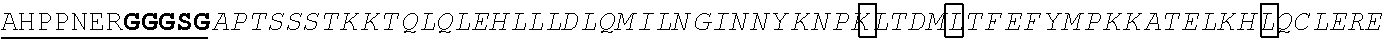

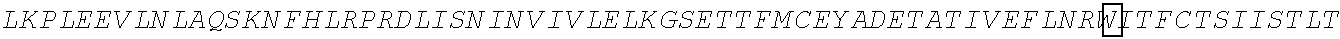

GVEVHNAKTKPREEQYNSTYRVVSVLTVLHQDWLNGKEYKCKVSNKALPAPIEKTISKAKGQPREPQVY

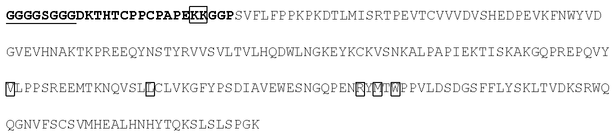

QGNVFSCSVMHEALHNHYTQKSLSLSPGK
SEQ ID NO: 161 (human PD-L1 extracellular domain mutant2 IS4Q/E58M/R113T/M115L/S117A/G119K
extracellular domain-linker-IL-2 mutant (L18R/Q22E/R38D/K43E/E61R/Q126T)-linker-hinge-IgG1 Fc
matant2; PD-L1 extracellular domain is underlined; linker is bolded and underlined; hinge is bolded;
IL-12 subunits are italicized)

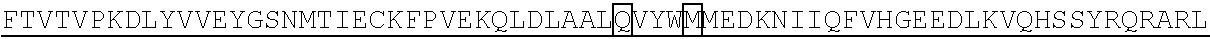

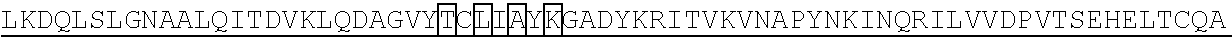

EGYPKAEVIWTSSDHOVLSGKTTTTNSKREEKLFNVTSTLRINTTTNEIFYCTFRRLDPEENHTAELVIPELPL

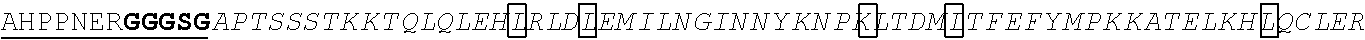

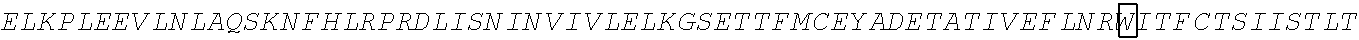

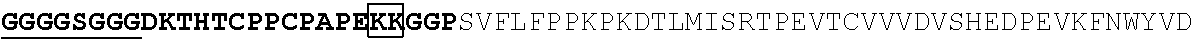

GVEVHNAKTKPREEQYNSTYRVVSVLTVLHQDWLNGKEYKCKVSNKALPAPIEKTISKAKGQPREPQVY

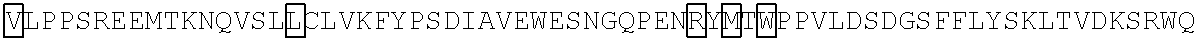

QGNVFSCSVMHEALHNHYTQKSLSLSPGK
SEQ ID NO: 162 (human PD-L1 extracellular domain mutant2 I54Q/E58M/R113T/M115L/S117A/G119K
extracellular domain-linker-IL-2 mutant (L18R/Q22E/R38D/K43E/E61R/Q126T/S130R)-linker-hinge-IgG1
Fc mutant2; PD-L1 extracellular domain is underlined; linker is bolded and underlined; hinge is
bolded; IL-12 subunits are italicized)

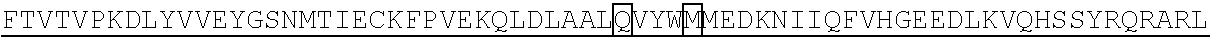

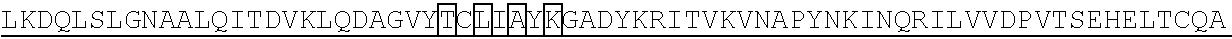

EGYPKAEVIWTSSDHQVLSGKTTTTNSKREEKLFNVTSTLRINTTTNEIFYCTFRRLDPEENHTAELVIPELPL

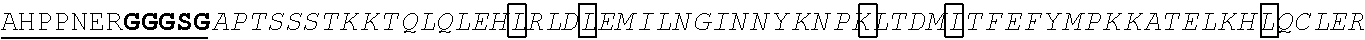

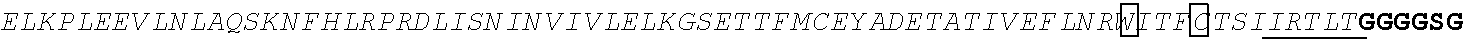

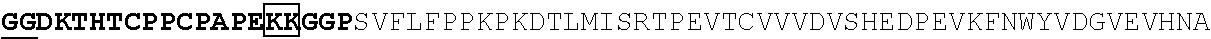

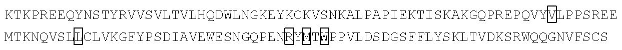

VMHEALHNHYTQKSLSLSPGK
SEQ ID NO: 163 (human PD-L1 extracellular domain mutant7 I54Q/E58M/R113T/M115L/S117A/G119K
extracellular domain-linker-IL-2 mutant (L18R/Q22E/R38D/K43E/E61R)-linker-hinge-IgG1 Fc mutant2;
PD-LI extracellular domain is anderlined; linker is bolded and underlined; hinge is bolded; IL-12
subunits are italicized)

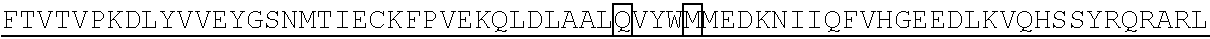

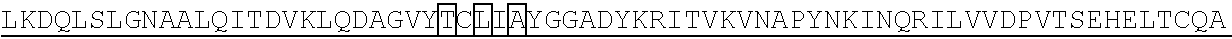

EGYPKAEVIWTSSDHQVLSGKTTTTNSKREEKLENVTSTLRINTTTNEIFYCTFRRLDPEENHTAELVIPELPL

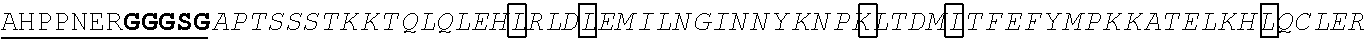

ELKPLEEVLNLAQSKNFHLRPRDLISNINVIVLELKGSETTFMCEYADETATIVEFLNRWITFCOSIISTLT

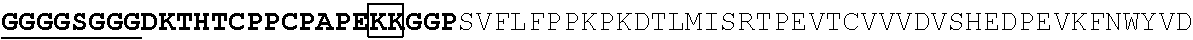

GVEVHNAKTKPREEQYNSTYRVVSVLTVLHQDWLNGKEYKCKVSNKALPAPIEKTISKAKGQPREPQVY

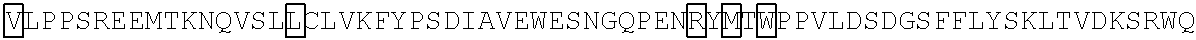

QGNVFSCSVMHEALHNHYTOKSLSLSPGK
SEQ ID NO: 164 (human PD-L1 extracellular domain mutant7 I54Q/E58M/R113T/M115L/S117A
extracellular domain-linker-IL-2 mutant (R38D/K43E/E61R/Q126T)-linker-hinge-IgG1 Fc mutant2; PD-L1
extracellular domain is underlined; linker is bolded and underlined; hinge is bolded; IL-12 subunits
are italicized)

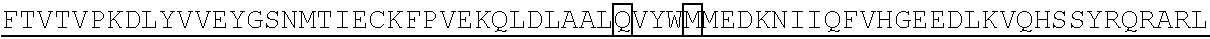

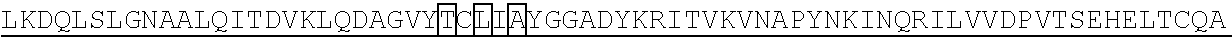

EGYPKAEVIWTSSDHQVLSGKTTTTNSKREEKLENVTSTLRINTTTNEIFYCTFRRLDPEENHTAELVIPELPL

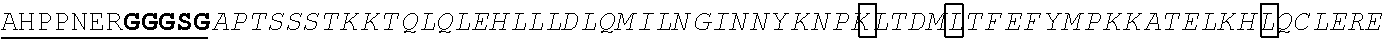

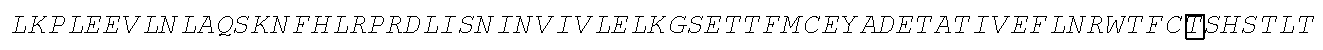

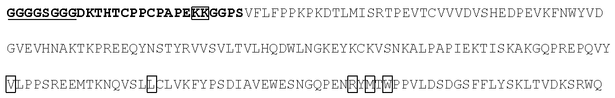

QGNVFSCSVMHEALHNHYTQKSLSLSPGK
SEQ ID NO: 165 (human PD-L1 extracellular domain mutant7 I54Q/E58M/R113T/M115L/S117A
extracellular domain-linker-IL-2 mutant (L18R/Q22E/R38D/K43E/E61R/Q126T)-linker-hinge-IgG1 Fc
mutant2; PD-L1 extracellular domain is underlined; linker is bolded and underlined; hinge is bolded;
IL-12 subunits are italicized)

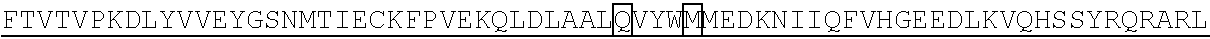

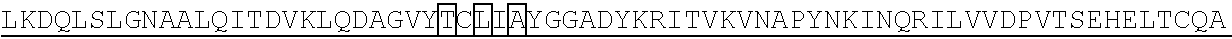

EGYPKAEVIWTSSDHQVLSGKTTTTNSKREEKLFNVTSTLRINTTTNEIFYCTERRLDPEENHTAELVIPELPL

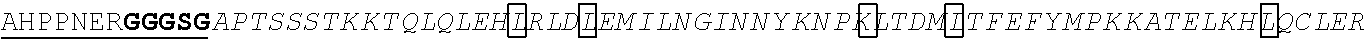

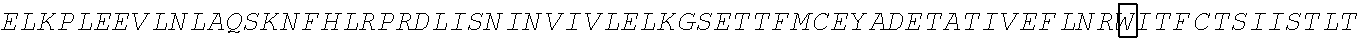

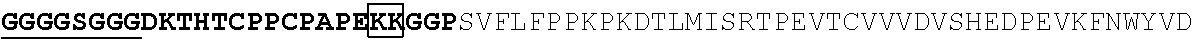

GVEVHNAKTKPREEQYNSTYRVVSVLTVLHQDWLNGKEYKCKVSNKALPAPIEKTISKAKGQPREPQVY

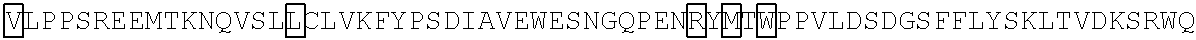

QGNVFSCSVMHEALHNHYTQKSLSLSPGK
SEQ ID NO: 166 (human PD-L1 extracellular domain mutant7 I54Q/E58M/R113T/M115L/S117A
extracellular domain-linker-IL-2 mutant (L18R/Q22E/R38D/K43E/E61R/Q126T/S130R)-linker-hinge-IgG1
Fc mutant2; PD-L1 extracellular domain is underlined; linker is bolded and underlined; hinge is bolded;
IL-12 subunits are italicized)

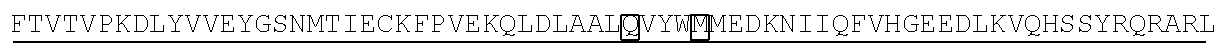

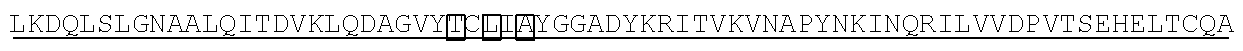

EGYPKAEVIWTSSDHQVLSGKTTTTNSKREEKLENVTSTLRINTTTNEIFYCTFRRLDPEENHTAELVIPELPL

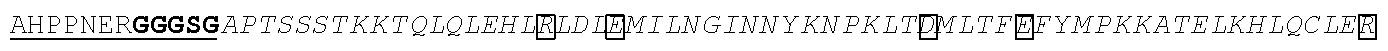

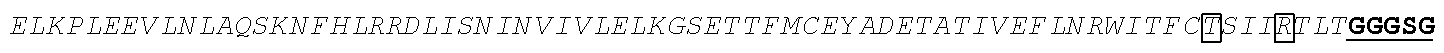

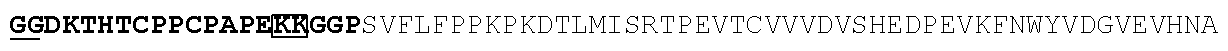

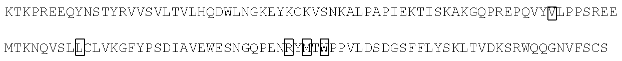

VMHEALHNHYTQKSLSLSPGK
SEQ ID NO: 167 (human PD-L2 extracellular domain mutant2 S58V extracellular domain-linker-single-
chain IL-12 mutant heterodimer IL~12B (p40 E59A/F60A)-linker IL-12A (wt p35)-hinge-IgG1 Fc mutant1;
PD-L2 extracellular domain is underlined; linker is bolded and underlined; hinge is bolded; IL-12
subunits are italicized)

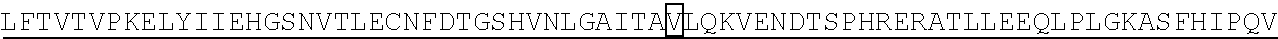

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNESCVFWNTHVRELTLASIDLQSQMEPRTHPTGGGGSGGGIWELKK

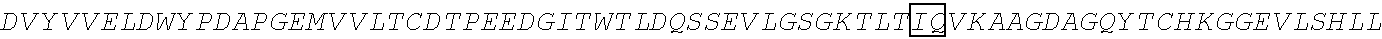

LLHKKEDGIWSTDILKDQKEPKNKTFLRCEAKNYSGRFTCWWLTTISTDLTFSVKSSRGSSDPQGVTCGAATLSAE
RVRGDNKEYEYSVECQEDSACPAAEESLPIEVMVDAVHKLKYENYTSSFFIRDIIKPDPPKNLQLKPLKNSRQVEVS
WEYPDTWSTPHSYFSLTFCVQVQGKSKREKKDRVFTDKTSATVICRKNASISVRAQDRYYSSSWSEWASVPCSGGG
GSGGGSGGGGSRNLPVATPDPGMFPCLHHSQNLLRAVSNMLQKAROTLEFYPCTSEEIDHEDITKDKTSTVEA
CLPLELTKNESCLNSRETSFITNGSCLASRKTSFMMALCLSSIYEDLKMYQVEFKTMNAKLLMDPKRQIFLDQNML
AVIDELMQALNFNSETVPQKSSLEEPDFYKTKIKLCILLHAFRIRAVTIDRVMSYLNASGGGGSGGGDKTHTCPP

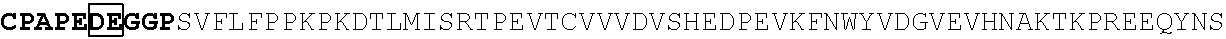

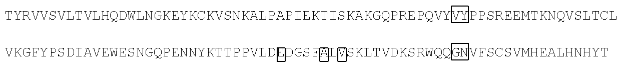

QKSLSLSPGK
SEQ ID NO: 168 (human PD-L2 extracellular domain mutant4 T56V/S58V/Q60L extracellular domain-
linker-single-chain IL-12 mutant heterodimer IL-12B (p40 E59A/F60A)-linker IL-12A (wt p35)-hinge-IgG1
Fc mutant1; PD-L2 extracellular domain is underlined; linker is bolded and underlined; binge is bolded;
IL-12 subunits are italicized)

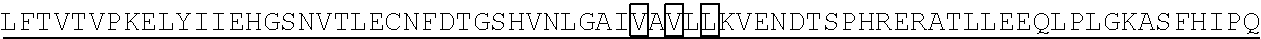

VQVRDEGQYQCHIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGGSGGGIWELK

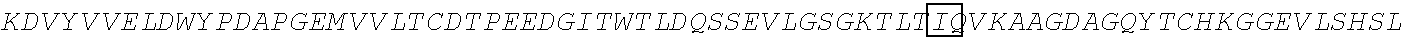

LLLHKKEDGIWSTDILKDQKEPKNKTFLRCEAKNYSGRFTCWWLTTISTDLTFSVKSSRGSSDPQGVTCGAATLSA
ERVRGDNKEYEYSVECQEDSACPAAEESLPIEVMVDAVHKLKYENYTSSFFIRDIIKPDPPKNLQLKPLKNSROVEV
SWEYPDTWSTPHSYFSLTFCVQVQGKSKREKKDRVFTDKTSATVICRKNASISVRAQDRYYSSSWSEWASVPCSGG
GGSGGGSGGGGSRNLPVATPDPGMFPCLHHSONLLRAVSNMLQKARQTLEFYPCTSEEIDHEDITKDKTSTVE
ACLPLELTKNESCLNSRETSFITNGSCLASRKTSFMMALCLSSIYEDLKMYQVEFKTMNAKLLMDPKRQIFLDQNM
LAVIDELMQALNFNSETVPQKSSLEEPDFYKTKIKLCILLHAFRIRAVTIDRVMSYLNASGGGGSGGGDKTHTCP

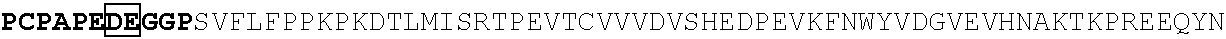

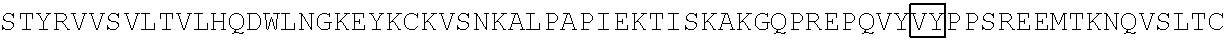

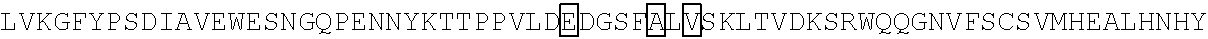

TQKSLSLSPGK
SEQ ID NO: 169 (human PD-L2 extracellular domain mutant2 S58V extracellular domain-linker-hinge-IgG1
Fc mutant1-linker-single-chain IL-12 mutant heterodimer IL-12B (p40 E59A/F60A)-linker IL-12A (wt p35);
PD-L2 extracellular domain is anderlined; linker is bolded and underlined; hinge is bolded; IL-12
subunits are italicized)

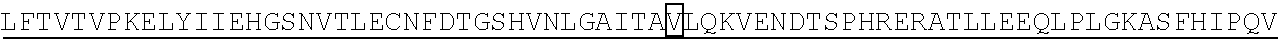

QVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSGGGDKTHT

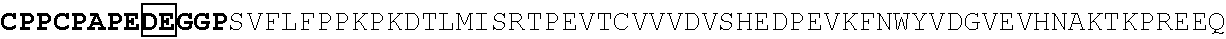

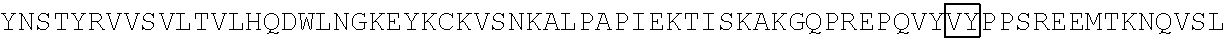

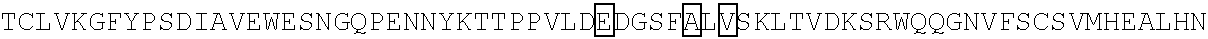

HYTQKSLSLSPGKGGGGSGGGIWELKKDVYVVELDWYPDAPGEMVVLTCDTPEEDGITWTLDQSSEVLGSGK

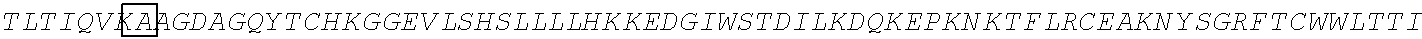

STDLTFSVKSSRGSSDPQGVTCGAATLSAERVRGDNKEYEYSVECQEDSACPAAEESLPIEVMVDAVHKLKYENYT
SSFFIRDIIKPDPPKNLQLKPLKNSRQVEVSWEYPDTWSTPHSYFSLTFCVQVQGKSKREKKDRVFTDKTSATVICR
KNASISVRAQDRYYSSSWSEWASVPCSGGGGSGGGSGGGGSRNLPVATPDPGMFPCLHHSQNLLRAVSNMLQK
ARQTLEFYPCTSEEIDHEDITKDKTSTVEACLPLELTKNESCLNSRETSFITNGSCLASRKTSFMMALCLSSIYEDLK
MYQVEFKTMNAKLLMDPKRQIFLDQNMLAVIDELMQALNFNSETVPQKSSLEEPDFYKTKIKLCILLHAFRIRAVT
IDRVMSYLNAS
SEQ ID NO: 170 (haman PD-L2 extracellular domain matant4 T56V/S58V/Q60L extracellular domain-
linker-hinge-IgG1 Fc mutant1-linker-single-chain IL-12 mutant heterodimer IL-12B (p40 E59A/F60A)-
linker IL-12A (wt p35); PD-L2 extracellular domain is underlined; linker is bolded and underlined;
hinge is bolded; IL-12 subunits are italicized)

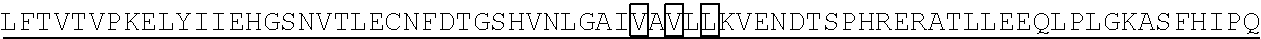

VQVRDEGQYQCIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGGSGGGDKTH

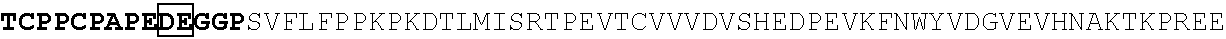

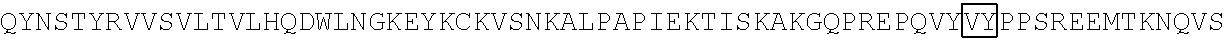

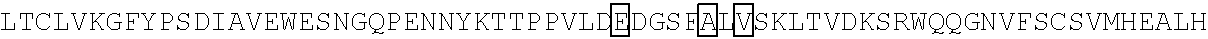

NHYTQKSLSLSPGKGGGGSGGGIWELKKDVYVVELDWYPDAPGEMVVLTCDTPEEDGITWTLDQSSEVLGSG

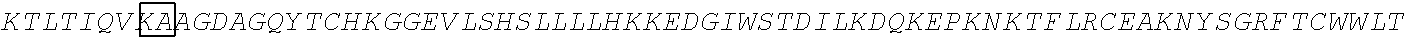

TISTDLTFSVKSSRGSSDPQGVTCGAATLSAERVRGDNKEYEYSVECQEDSACPAAEESLPIEVMVDAVHKLKYENY
TSSFFIRDIIKPDPPKNLQLKPLKNSRQVEVSWEYPDTWSTPHSYFSLTFCVQVQGKSKREKKDRVFTDKTSATVIC
RKNASISVRAQDRYYSSSWSEWASVPCSGGGGSGGGSGGGGSRNLPVATPDPGMFPCLHHSQNLLRAVSNMLQ
KARQTLEFYPCTSEEIDHEDITKDKTSTVEACLPLELTKNESCLNSRETSFITNGSCLASRKTSFMMALCLSSIYEDL
KMYQVEFKTMNAKLLMDPKRQIFLDQNMLAVIDELMQALNENSETVPQKSSLEEPDFYKTKIKLCILLHAFRIRA
VTIDRVMSYLNAS
SEQ ID NO: 171 (human PD-L2 extracellular domain mutant2 858V extracellular domain-linker-IL-2
matant (L18R/Q22E/R38D/K43E/E61R)-linker-hinge-IgG1 Fc mutant2; PD-L2 extracellular domain is
underlined; linker is bolded and underlined; hinge is bolded; IL-12 subunits are italicized)

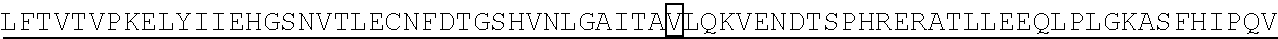

QVRDEGQYQCIIIYGVAWDYKYLILKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGSGAPTSSSTKKT

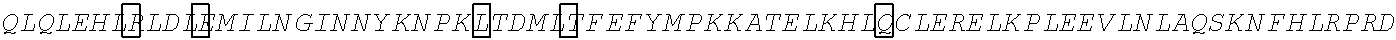

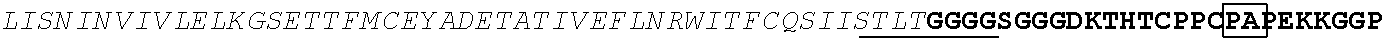

SVFLFPPKPKDTLMISRTPEVTCVVVDVSHEDPEVKFNWYVDGVEVHNAKTKPREEQYNSTYRVVSVLTV

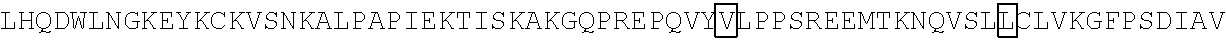

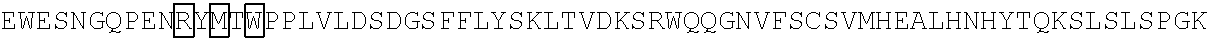

SEQ ID NO: 172 (human PD-L2 extracellular domain mutant2 S58V extracellular domain-linker-IL-2
mutant (R38D/K43E/E61R/Q126T)-linker-hinge-IgG1 Fc mutant2; PD-L2 extracellular domain is
underlined; linker is bolded and underlined; binge is bolded; IL-12 subunits are italicized)

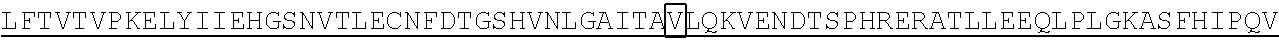

QVRDEGQYQCIIIYGVAWDYKYLTLKVKASYRKINTHILKVPETDEVELTCQATGYPLAEVSWPNVSVPAN
TSHSRTPEGLYQVTSLRLKPPPGRNFSCVFWNTHVRELTLASIDLQSQMEPRTHPTGGGSGAPTSSSTKKTQL

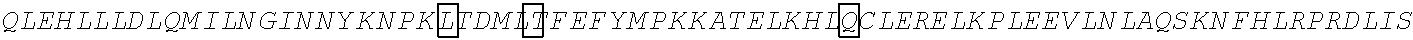

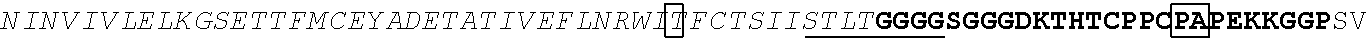

FLFPPKPKDTLMISRTPEVTCVVVDVSHEDPEVKFNWYVDGVEVHNAKIKPREEQYNSTYRVVSVLTVLH

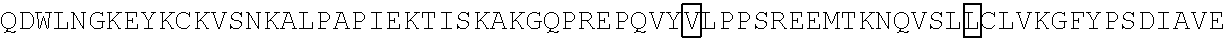

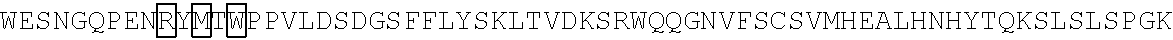

SEQ ID NO: 173 (human PD-L2 extracellular domain mutant2 858V extracellular domain-linker-IL-2
mutant (L18R/Q22E/R38D/K43E/E61R/Q126T)-linker-hinge-IgG1 Fc mutant2; PD-L2 extracellular domain
is underlined; linker is bolded and underlined; hinge is bolded; IL-12 subunits are italicized)

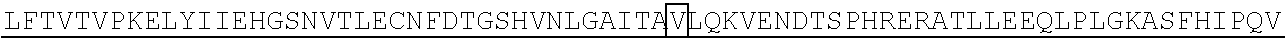

QVRDEGQYQCIIIYGVAWDYKYLTLKVKASYRKINTHILKVPETDEVELTCQATGYPLAEVSWPNVSVPAN
TSHSRTPEGLYQVTSLRLKPPPGRNFSCVFWNTHVRELTLASIDLQSQMEPRTHPTGGGSGAPTSSSTKKTQL

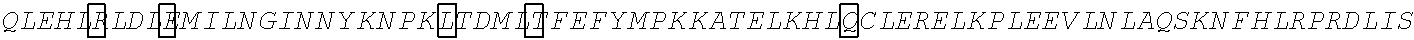

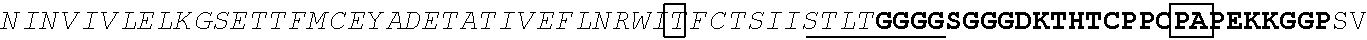

FLFPPKPKDTLMISRTPEVTCVVVDVSHEDPEVKFNWYVDGVEVHNAKTKPREEQYNSTYRVVSVLTVLH

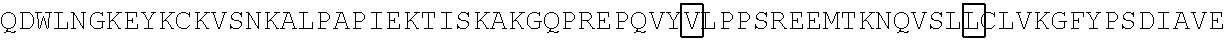

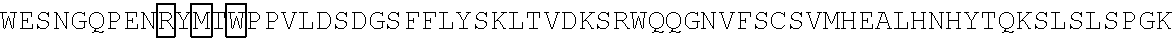

SEQ ID NO: 174 (human PD-L2 extracellular domain mutant2 S58V extracellular domain-linker-IL-2
mutant (R38D/K43E/E61R)(L18R/Q22E/Q126T/S130R)-linker-binge-IgG1 Fc mutant2; PD-L2 extracellular
domain is underlined; linker is bolded and underlined; hinge is bolded; IL-12 subunits are italicized)

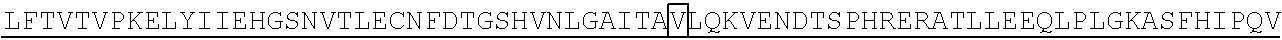

QVRDEGQYQCIIIYGVAWDYKYLTLKVKASYRKINTHILKVPETDEVELTCQATGYPLAEVSWPNVSVPAN
TSHSRTPEGLYQVTSLRLKPPPGRNESCVFWNTHVRELTLASIDLQSQMEPRTHPTGGGSGAPTSSSTKKTQL

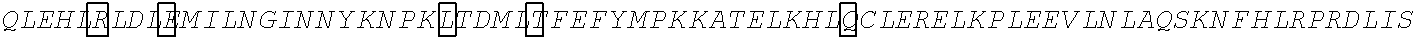

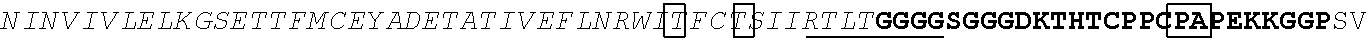

FLFPPKPKDTLMISRTPEVTCVVVDVSHEDPEVKFNWYVDGVEVHNAKTKPREEQYNSTYRVVSVLTVLH

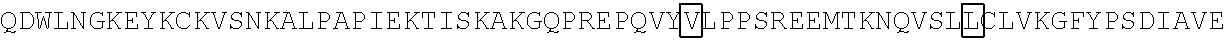

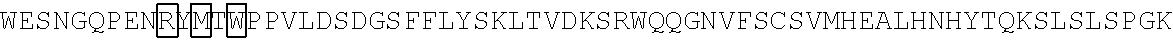

SEQ ID NO: 175 (human PD-L2 extracellular domain mutant4 T56V/S58V/Q60L extracellular domain-
linker-IL-2 mutant (L18R/Q22E/R38D/K43E/E61R)-linker-hinge-IgG1 Fc mutant2; PD-L2 extracellular
domain is underlined; linker is bolded and underlined; hinge is bolded; IL-12 subunits are italicized)

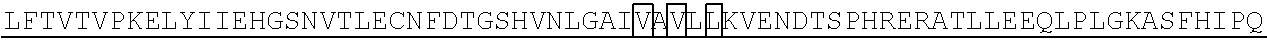

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGAPTSSSTKK

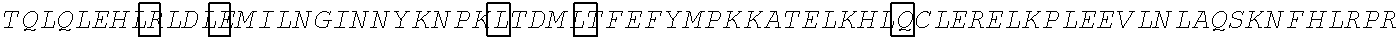

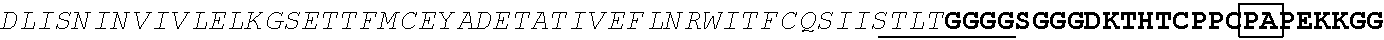

PSVFLFPPKPKDTLMISRTPEVTCVVVDVSHEDPEVKFNWYVDGVEVHNAKTKPREEQYNSTYR VVSVLT

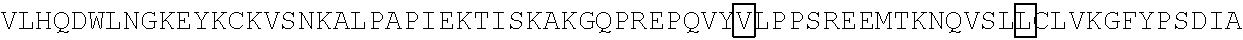

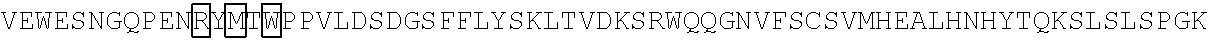

SEQ ID NO: 176 (human PD-L2 extracellular domain mutant4 T56V/S58V/Q60L extracellular domain-
linker-IL-2 mutant (R38D/K43E/E61R/Q126T)-linker-hinge-IgG1 Fc mutant2; PD-L2 extracellular domain
is underlined; linker is bolded and underlined; hinge is bolded; IL-12 subunits are italicized)

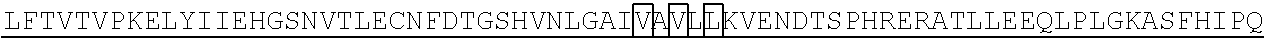

VQVRDEGOYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGAPTSSSTKK

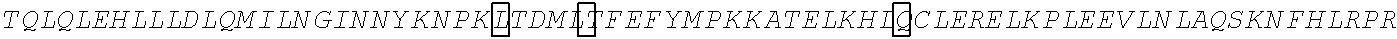

PSVFLFPPKPKDTLMISRTPEVTCVVVDVSHEDPEVKFNWYVDGVEVHNAKTKPREEQYNSTYRVVSVLT

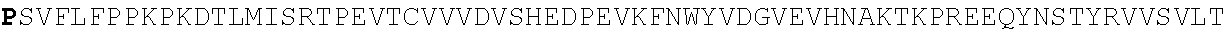

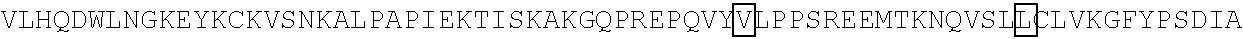

SEQ ID NO: 177 (human PD-L2 mutant4 T56V/S58V/Q60L extracellular domain-linker-IL-2 mutant
(L18R/Q22E/R38D/K43E/E61R/Q126T)-linker-hinge-IgG1 Fc mutant2; PD-L2 extracellular domain is
underlined; linker is bolded and underlined; binge is bolded; IL-12 subanits are italicized)

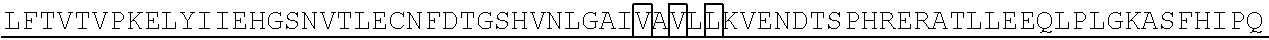

VQVRDEGQYQCIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGAPTSSSTKK

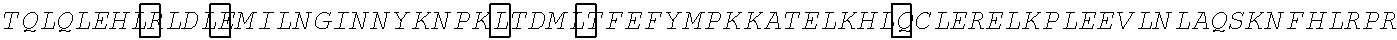

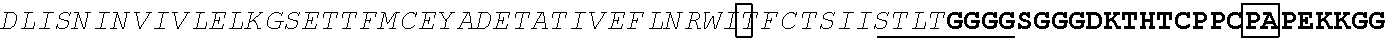

PSVFLFPPKPKDTLMISRTPEVTCVVVDVSHEDPEVKFNWYVDGVEVHNAKTKPREEQYNSTYRVVSVLT

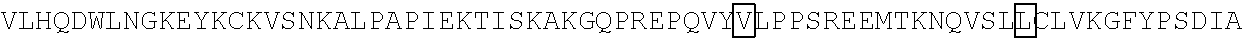

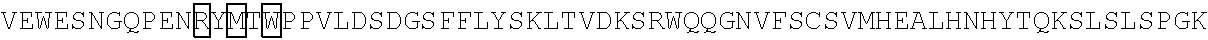

SEQ ID NO: 178 (human PD-L2 mutant4 T56V/S58V/Q60L extracellular domain-linker-IL-2 mutant
(L18R/Q22E/R38D/K43E/E61R/Q126T/S130R)-linker-hinge-IgG1 Fc mutant2; PD-L2 extracellular domain
is underlined; linker is bolded and underlined; hinge is bolded; IL-12 subunits are italicized)

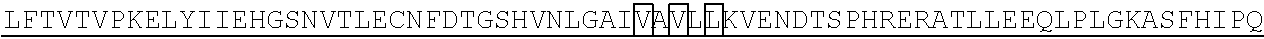

VQVRDEGQYQCIIIYGVAWDYKYLTLKVKASYRKINTHILKVPETDEVELTCQATGYPLAEVSWPNVSVPA
NTSHSRTPEGLYQVTSVLRLKPPPGRNFSCVFWNTHVRELTLASIDLQSQMEPRTHPTGGGSGAPTSSSTKK

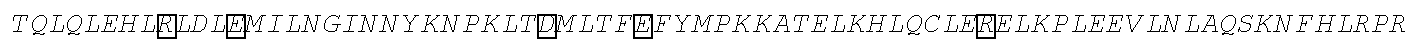

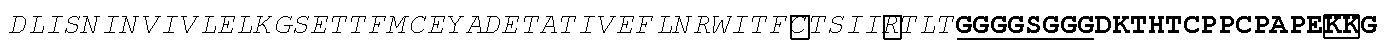

GPSVFLFPPKPKDTLMISRTPEVTCVVVDVSHEDPEVKFNWYVDGVEVHNAKTKPREEQYNSTYRVVSVL

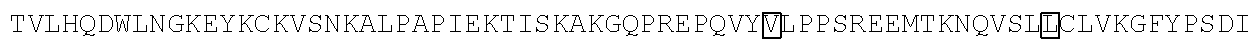

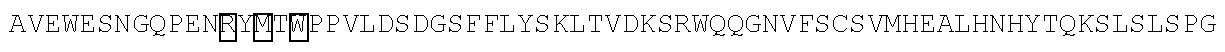

K
SEQ ID NO: 179 (human PD-L1 matant2 I54Q/E58M/R113T/M115L/S117A/G119K extracellular domain-
linker-IL-2 mutant (R38D/K43E/E61R) linker-hinge-IgG1 Fe mutant2; PD-L1 extracellular domain is
underlined; linker is bolded and underlined; hinge is bolded; IL-12 subunits are italicized)

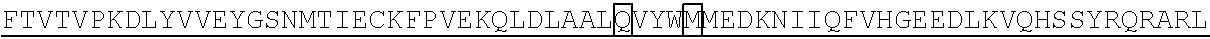

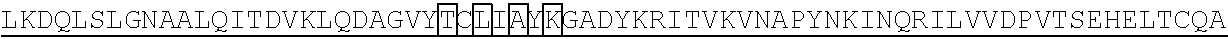

EGYPKAEVIWTSSDHQVLSGKTTTTNSKREEKLFNVTSTLRINTTTNEIFYCTFRRLDPEENHTAELVIPELPL

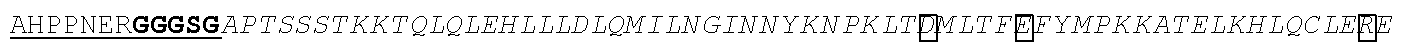

LKPLEEVLNLAQSKNFHLRPRDLISNINVIVLELKGSETTFMCEYADETATIVEFLNRWITFCQSIISTLT

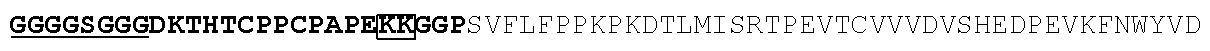

GVEVHNAKTKPREEQYNSTYRVVSVLTVLHQDWLNGKEYKCKVSNKALPAPIEKTISKAKGQPREPQVY

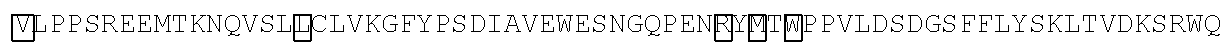

QGNVFSCSVMHEALHNHYTQKSLSLSPGK
SEQ ID NO: 180 (human PD-L1 extracellular domain mutant2 I54Q/E58M/R113T/M115L/S117A/G119K
extracellular domain-linker-single-chain mouse IL-12 mutant heterodimer IL-12B (p40 E59A/F60A)-linker
IL~12A (wt p35)-hinge-IgG1 Fc mutant1; PD-L1 extracellular domain is underlined; linker is bolded and
underlined; hinge is bolded; mouse IL-12 subunits are italicized)

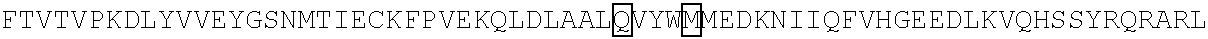

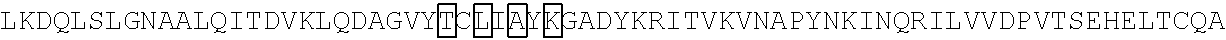

EGYPKAEVIWTSSDHQVLSGKTTTTNSKREEKLFNVTSTLRINTTTNEIFYCTFRRLDPEENHTAELVIPELPL
AHPPNERGGGGSGGGMWELEKDVYVVEVDWTPDAPGETVNLTCDTPEEDDITWTSDQRHGVIGSGKTLTTIV

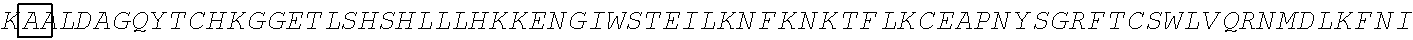

KSSSSSPDSRAVTCGMASLSAEKVTLDQRDYEKYSVSCQEDVTCPTAEETLPIELALEARQQNKYENYSTSFFIRDII
KPDPPKNLQMKPLKNSQVEVSWEYPDSWSTPHSYFSLKFFVRIQRKKEKMKETEEGCNQKGAFLVEKTSTEVQC
KGGNVCVQAQDRYYNSSCSKWACVPCRVRSGGPGGGGSGGGSGGGGSGRVIPVSGPARCLSQSRNLLKTTDD
MVKTAREKLKHYSCTAEDIDHEDITRDQTSTLKTCLPLELHKNESCLATRETSSTTRGSCLPPQKTSLMMTLCLGSI
YEDLKMYQTEFQAINAALQNHNHQQIILDKGMLVAIDELMQSLNHNGETLROKPPVGEADPYRVKMKLCILLHA

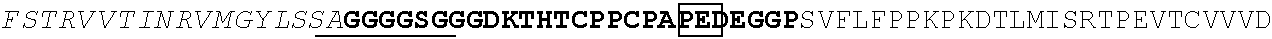

VSHEDPEVKFNWYVDGVEVHNAKTKPREEQYNSTYRVVSVLTVLHQDWLNGKEYKCKVSNKALPAPIEK

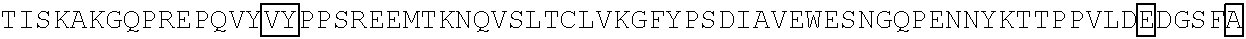

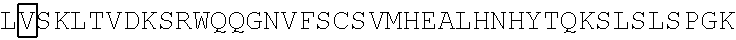

SEQ ID NO: 181 (human PD-L1 extracellular domain mutant2 I54Q/E58M/R113T/M115L/S117A/G119K
extracellular domain-linker-hinge-IgG1 Fc mutant1- linker-single-chain mouse IL-12 mutant heterodimer
IL-12B (p40 E59A/F60A)-linker IL-12A (wt p35); PD-L1 extracellular domain is underlined; linker is
bolded and underlined; hinge is bolded; mouse IL-12 subunits are italicized)

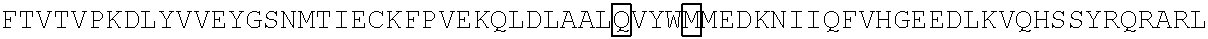

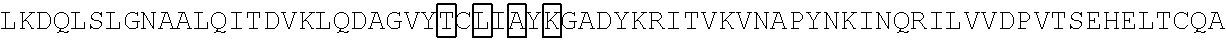

EGYPKAEVIWTSSDHQVLSGKTTTTNSKREEKLFNVTSTLRINTTTNEIFYCTFRRLDPEENHTAELVIPELPL

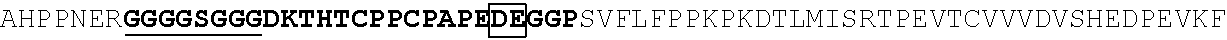

NWYVDGVEVHNAKTKPREEQYNSTYRVVSVLTVLHQDWLNGKEYKCKVSNKALPAPIEKTISKAKGQPR

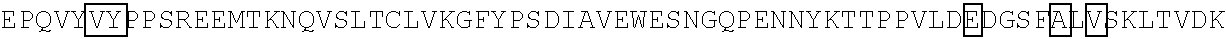

SRWQQGNVFSCSVMHEALHNHYTQKSLSLSPGKGGGGSGGGMWELEKDVYVVEVDWTPDAPGETVNLTC

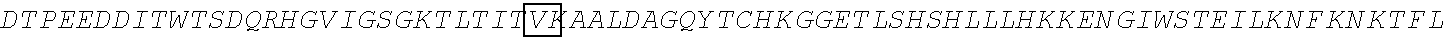

KCEAPNYSGRFTCSWLVQRNMDLKFNIKSSSSSPDSRAVTCGMASLSAEKVTLDQRDYEKYSVSCOEDVTCPTAEE
TLPIELALEARQQNKYENYSTSFFIRDIIKPDPPKNLQMKPLKNSQVEVSWEYPDSWSTPHSYFSLKFFVRIORKKE
KMKETEEGCNQKGAFLVEKTSTEVQCKGGNVCVQAQDRYYNSSCSKWACVPCRVRSGGPGGGGSGGGSGGG
GSGRVIPVSGPARCLSOSRNLLKTTDDMVKTAREKLKHYSCTAEDIDHEDITRDQTSTLKTCLPLELHKNESCLAT
RETSSTTRGSCLPPQKTSLMMTLCLGSIYEDLKMYQTEFQAINAALQNHNHQQIILDKGMLVAIDELMQSLNHNG
ETLRQKPPVGEADPYRVKMKLCILLHAFSTRVVTINRVMGYLSSA
SEQ ID NO: 182 (human PD-L1 extracellular domain mutant2 I54Q/E58M/R113T/M115L/S117A/G119K
extracellular domain-linker-single-chain mutant homodimer IFN-γ (A23V/A23V)-linker hinge-IgG1 Fc
mutant1; PD-L1 extracellular domain is underlined; linker is bolded and underlined; hinge is bolded;
IFN-γ is italicized)

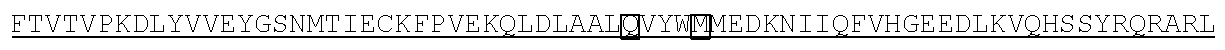

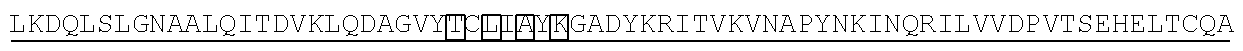

EGYPKAEVIWTSSDHOVLSGKTTTTNSKREEKLENVTSTLRINTTTNEIFYCTFRRLDPEENHTAELVIPELPL

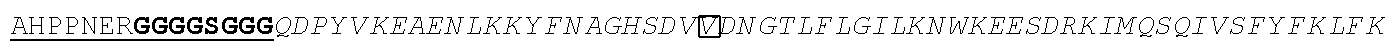

NFKDDQSIQKSVETIKEDMNVKFFNSNKKKRDDFEKLTNYSVTDLNVQRKAIHELIQVMAELSPAAKTGKRKRSQ

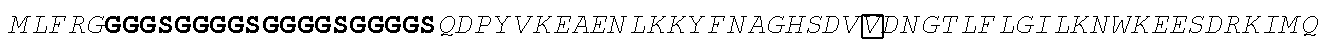

SQIVSFYFKLEKNEKDDQSIQKSVETIKEDMNVKFFNSNKKKRDDFEKLTNYSVTDLNVQRKAIHELIQVMAELSP

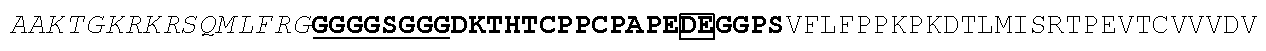

SHEDPEVKFNWYVDGVEVHNAKTKPREEQYNSTYRVVSVLTVLHQDWLNGKEYKCKVSNKALPAPIEKT

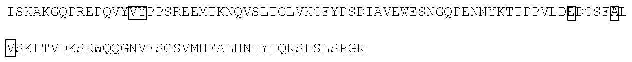

SEQ ID NO: 183 (human PD-L1 extracellular domain mutant7 I54Q/E58M/R113T/M115L/S117A
extracellular domain-linker- single-chain mutant homodimer IFN-γ (A23V/A23V)-linker hinge-IgGI Fe
mutant1; PD-L1 extracellular domain is underlined; linker is bolded and underlined; hinge is bolded;
IFN-γ is italicized)

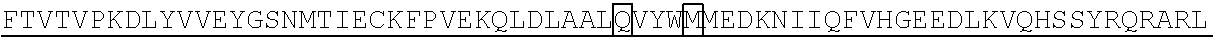

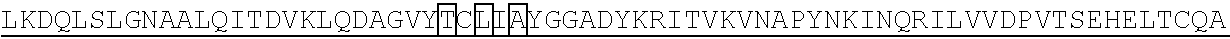

EGYPKAEVIWTSSDHQVLSGKTTTTNSKREEKLFNVTSTLRINTTTNEIFYCTFRRLDPEENHTAELVIPELPL

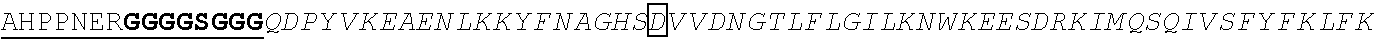

NFKDDQSIQKSVETIKEDMNVKFFNSKKKRDDFEKLTNYSVTDLNVQRKAIHELIQVMAELSPAAKTGKRKRSQ

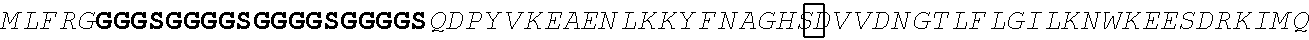

SQIVSFYFKLFKNFKDDQSIQKSVETIKEDMNVKFFNSNKKKRDDFEKLTNYSVTDLNVQRKAIHELIOVMAELSP

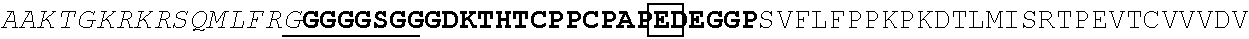

SHEDPEVKFNWYVDGVEVHNAKTKPREEQYNSTYRVVSVLTVLHQDWLNGKEYKCKVSNKALPAPIEKT

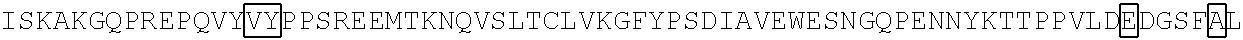

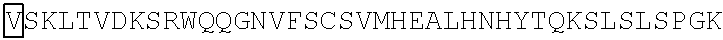

SEQ ID NO: 184 (human PD-L1 extracellular domain mutant2 I54Q/E58M/R113T/M115L/S117A/G119K
extracellular domain-linker-single-chain mutant homodimer IFN-γ (A23V/A23V)-linker-hinge-IgG1 Fc
mutant2; PD-L1 is extracellular domain underlined; linker is shaded; hinge is bolded; IFN-γ is
italicized)

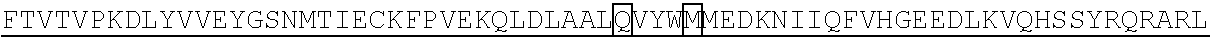

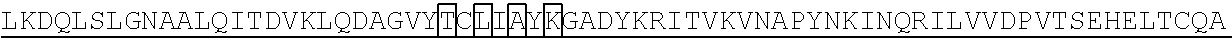

EGYPKAEVIWTSSDHQVLSGKTTTTNSKREEKLFNVTSTLRINTTTNEIFYCTERRLDPEENHTAELVIPELPL

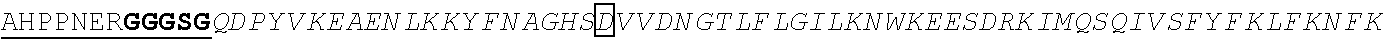

DDQSIQKSVETIKEDMNVKFFNSNKKKRDDFEKLINYSVTDLNVQRKAIHELIQVMAELSPAAKTGKRKRSQMLF

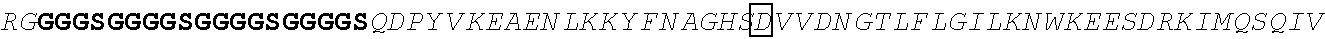

SFYFKLFKNFKDDQSIQKSVETIKEDMNVKFFNSNKKKRDDFEKLINYSVTDLNVQRKAIHELIQVMAELSPAAKT

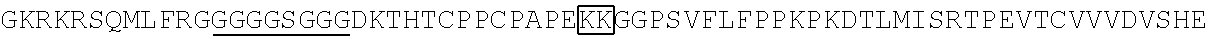

DPEVKFNWYVDGVEVHNAKTKPREEQYNSTYRVVSVLTVLHQDWLNGKEYKCKVSNKALPAPIEKTISK

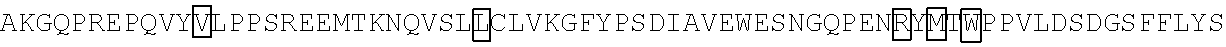

KLTVDKSRWQQGNVFSCSVMHEALHNHYTQKSLSLSPGK
SEQ ID NO: 185 (human PD-L1 extracellular domain mutant7 I54Q/E58M/R113T/M115L/S117A/G119K
extracellular domain-linker-single-chain mutant homodimer IFN-γ (A23V/A23V)-linker-hinge-IgG1 Fc
mutant2; PD-L1 extracellular domain is underlined; linker is bolded and underlined; hinge is bolded;
IFN-γ is italicized)

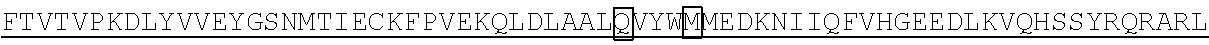

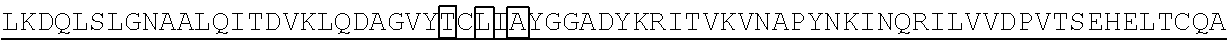

EGYPKAEVIWTSSDHQVLSGKTTTTNSKREEKLFNVTSTLRINTTTNEIFYCTFRRLDPEENHTAELVIPELPL

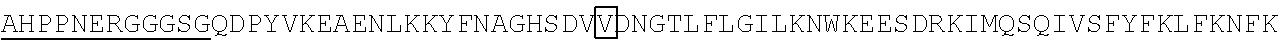

DDQSIQKSVETIKEDMNVKFFNSNKKKRDDFEKLTNYSVTDLNVQRKAIHELIQVMAELSPAAKTGKRKRSQMLF

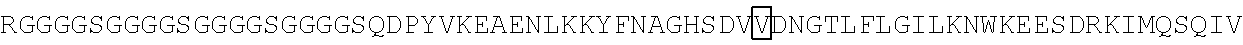

SFYFKLFKNFKDDOSIQKSVETIKEDMNVKFFNSNKKKRDDFEKLTNYSVTDLNVQRKAIHELIQVMAELSPAAKT

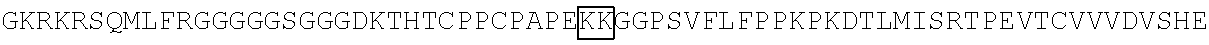

DPEVKFNWYVDGVEVHNAKTKPREEQYNSTYRVVSVLTVLHQDWLNGKEYKCKVSNKALPAPIEKTISK

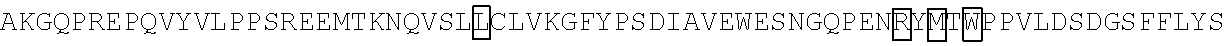

KLTVDKSRWQQGNVFSCSVMHEALHNHYTQKSLSLSPGK
SEQ ID NO: 186 (human PD-L2 extracellular domain mutant2 S58V extracellular domain-linker-single-
chain mutant homodimer IFN-γ (A23V/A23V)-linker-hinge-IgG1 Fc mutant1; PD-L2 extracellular domain is
underlined; linker is bolded and underlined; hinge is bolded; IFN-γ is italicized)

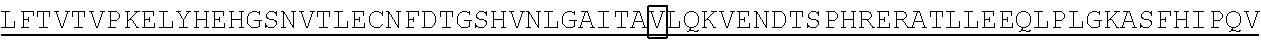

QVRDEGOYOCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTGGGGSGGGQDPYVK

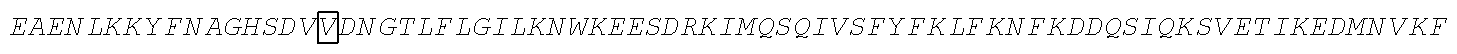

FNSNKKKRDDFEKLTNYSVTDLNVORKAIHELIQVMAELSPAAKTGKRKRSQMLFRGGGGGGGGSGGGGSG

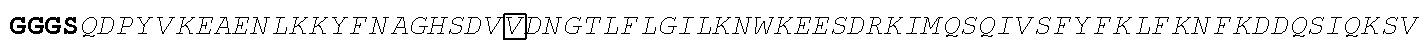

ETIKEDMNVKFFNSNKKKRDDFEKLINYSVTDLNVQRKAIHELIQVMAELSPAAKTGKRKRSOMLFRGGGGGSG

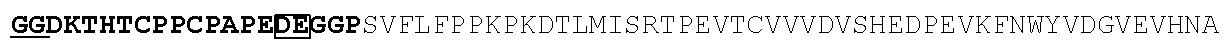

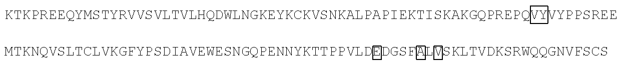

VMHEALHNHYTQKSLSLSPGK
SEQ ID NO: 187 (human PD-L2 extracellular domain mutant4 T56V/S58V/Q60L extracellular domain-
linker- single-chain mutant homodimer IFN-γ (A23V/A23V)-linker-hinge-IgG1 Fc mutant1; PD-L2
extracellular domain is underlined; linker is bolded and underlined; hinge is bolded; IFN-γ is
italicized)

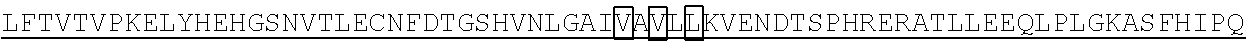

VQVRDEGQYQCIIIYGVAWDYKYLTLKVKASYRKINTHILKVPETDEVELTQOATGYPLAEVSWPNVSVPA
NTSHSRTPEGLYQVTSVLRLKPPPGRNFSCVFWNTHVRELTLASIDLOSOMEPRTHPTGGGGSGGGQDPYV

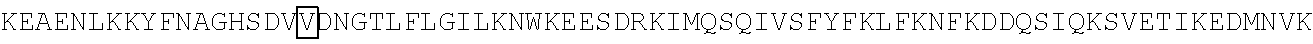

FFNSNKKKRDDFEKLTNYSVTDLNVQRKAIHELIQVMAELSPAAKTGKRKRSQMLFRGGGGSGGGGSGGGGS

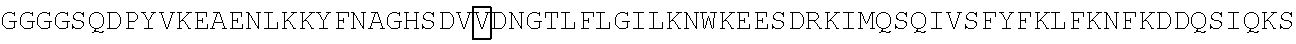

VETIKEDMNVKFFNSNKKKRDDFEKLINYSVTDLNVORKAIHELIQVMAELSPAAKTGKRKRSQMLFRGGGGGS

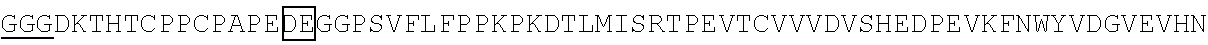

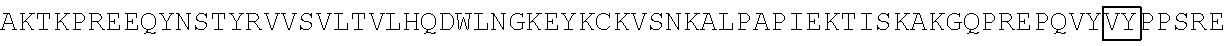

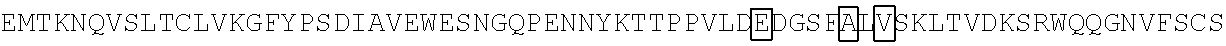

VMHEALHNHYTQKSLSLSPGK
SEQ ID NO: 188 (human PD-L2 extracellular domain mutant2 858V extracellular domain-linker-single-
chain mutant homodimer IFN-γ (A23V/A23V)-linker-hinge-IgG1 Fc mutant2; PD-L2 extracellular domain is
underlined; linker is bolded and underlined; hinge is bolded; IFN-γ is italicized)

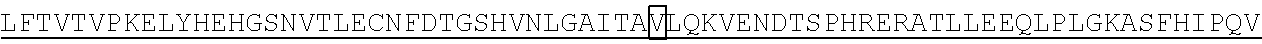

QVRDEGQYQCIIIYGVAWDYKYLTLKVKASYRKINTHILKVPETDEVELTCQATGYPLAEVSWPNVSVPAN
TSHSRTPEGLYOVTSVLRLKPPPGRNFSCVFWNTHVRELTLASIDLQSQMEPRTHPTGGGSGQDPYVKEAEN

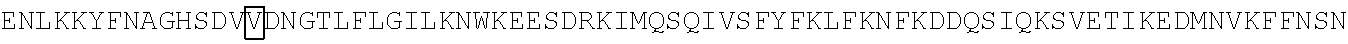

KKKRDDFEKLTNYSVTDLNVQRKAIHELIQVMAELSPAAKTGKRKRSQMLFRGGGGGGGGSGGGGSGGGG

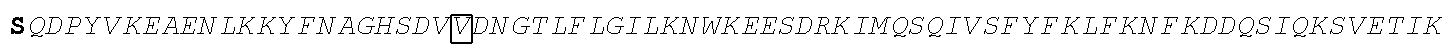

EDMNVKFFNSNKKKRDDFEKLTNYSVTDLNVQRKAIHELIQVMAELSPAAKTGKRKRSQMLFRGGGGGSGGG

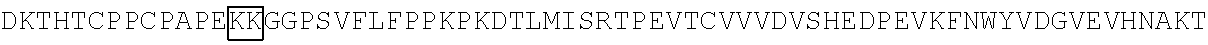

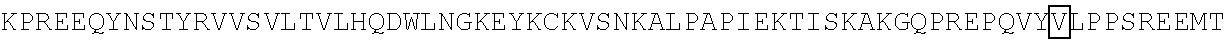

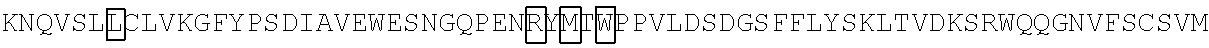

HEALHNHYTQKSLSLSPGK
SEQ ID NO: 189 (human PD-L2 extracellular domain mutant4 T56V/S58V/Q60L extracellular domain-
linker-single-chain mutant homodimer IFN-γ (A23V/A23V)-linker-hinge-IgG1 Fc mutant2; PD-L2
extracellular domain is underlined; linker is bolded and underlined; hinge is bolded; IFN-γ is
italicized)

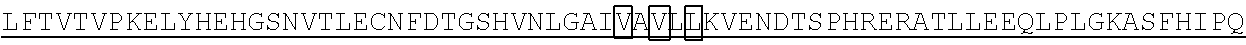

VQVRDEGOYQCIIIYGVAWDYKYLTLKVKASYRKINTHILKVPETDEVELTCQATGYPLAEVSWPNVSVPA
NTSHSRTPEGLYOVTSVLRLKPPPGRNFSCVFWNTHVRELTLASIDLQSOMEPRTHPTGGGSGQDPYVKEA

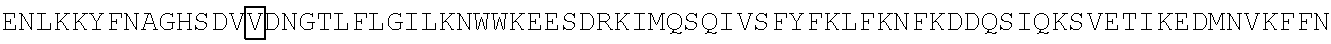

SNKKKRDDFEKLINYSVTDLNVQRKAIHELIQVMAELSPAAKTGKRKRSOMLERGGGGSGGGGSGGGGSGGG

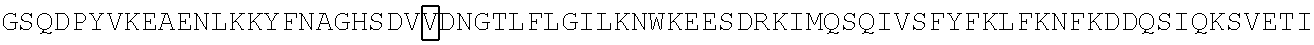

KEDMNVKFFNSNKKKRDDFEKLTNYSVTDLNVORKAIHELIQVMAELSPAAKTGKRKRSQMLFRGGGGGSGG

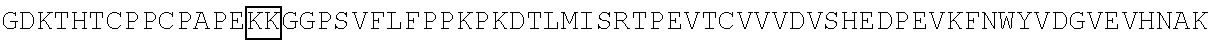

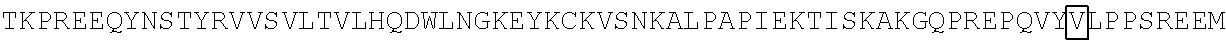

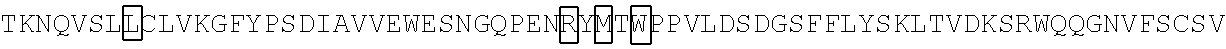

MHEALHNHYTOKSLSLSPGK
SEQ ID NO: 190 (human CD155 extracellular domain-linker-single-chain IL-12 mutant heterodimer IL-12B
(p40 E59A/F60A)-linker IL-12A (wt p35)-hinge-IgG1 Fc mutant1; CD155 extracellular domain is underlined;
linker is bolded and underlined; hinge is bolded; IL-12 subunits are italicized)
WPPPGTGDVVVQAPTQVPGFLGDSVTLPCYLQVPNMEVTHVSQLTWARHGESGSMAVFHOTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP
VDKPINTTLICNVTNALGARQAELTVQVKEGPPSEHSGISRNGGGGSGGGIWELKKDVYVVELDWYPDAPG

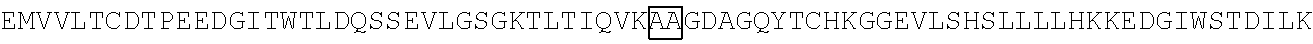

DQKEPKNKTFLRCEAKNYSGRFTCWWLTTISTDLTFSVKSSRGSSDPQGVTCGAATLSAERVRGDNKEYEYSVEC
QEDSACPAAEESLPIEVMVDAVHKLKYENYTSSFFIRDIIKPDPPKNLQLKPLKNSRQVEVSWEYPDTWSTPHSYES
LTFCVQVQGKSKREKKDRVFTDKTSATVICRKNASISVRAQDRYYSSSWSEWASVPCSGGGGSGGGSGGGGSRN
LPVATPDPGMFPCLHHSONLLRAVSNMLOKAROTLEFYPCTSEEIDHEDITKDKTSTVEACLPLELTKNESCLNSR
ETSFITNGSCLASRKTSFMMALCLSSIYEDLKMYOVEFKTMNAKLLMDPKRQIFLDQNMLAVIDELMQALNENSE

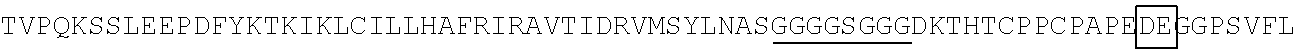

FPPKPKDTLMISRTPEVTCVVVDVSHEDPEVKFNWYVDGVEVHNAKTKPREEQYNSTYRVVSVLTVLHQD

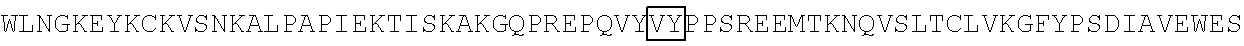

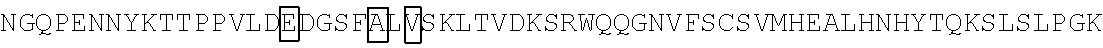

SEQ ID NO: 191 (human CD155 extracellular domain-linker-hinge-IgG1 Fc mutant1-linker-single-chain IL-
12 mutant heterodimer IL-12B (p40 E59A/F60A)-linker IL-12A (wt p35); CD155 extracellular domain is
underlined; linker is bolded and underlined; hinge is bolded; IL-12 subunits are italicized)
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGONEATLTCDARSNPEPTGYNWSTTMGPLPPFAVAQGAQLLIRP

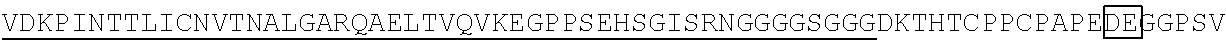

FLFPPKPKDTLMISRTPEVTCVVVDVSHEDPEVKFNWYVDGVEVHNAKTKPREEQYNSTYRVVSVLTVLH

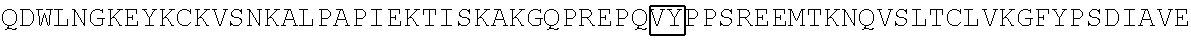

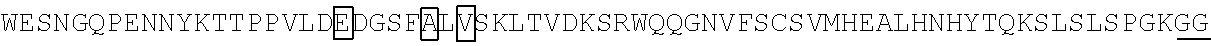

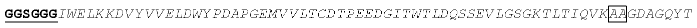

CHKGGEVLSHSLLLLHKKEDGIWSTDILKDQKEPKNKTFLRCEAKNYSGRFTCWWLTTISTDLTFSVKSSRGSSDP
QGVTCGAATLSAERVRGDNKEYEYSVECOEDSACPAAEESLPIEVMVDAVHKLKYENYTSSFFIRDIIKPDPPKNL
QLKPLKNSRQVEVSWEYPDTWSTPHSYFSLTFCVQVQGKSKREKKDRVFTDKTSATVICRKNASISVRAQDRYYSSS
WSEWASVPCSGGGGSGGGSGGGGSRNLPVATPDPGMFPCLHHSONLLRAVSNMLQKARQTLEFYPCTSEEID
HEDITKDKTSTVEACLPLELTKNESCLNSRETSFITNGSCLASRKTSFMMALCLSSIYEDLKMYQVEFKTMNAKLLM
DPKRQIFLDQNMLAVIDELMQALNFNSETVPQKSSLEEPDFYKTKIKLCILLHAFRIRAVTIDRVMSYLNAS
SEQ ID NO: 192 (human CD155 extracellular domain-linker-single-chain mutant homodimer IFN-γ
(A23V/A23V)-linker hinge-IgG1 Fc mutant1; CD155 extracellular domain is underlined; linker is bolded
and underlined; hinge is bolded; IFN-γ is italicized)
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP
VDKPINTTLICNVTNALGARQAELTVQVKEGPPSEHSGISRNGGGGSGGGQDPYVKEAENLKKYFNAGHSD

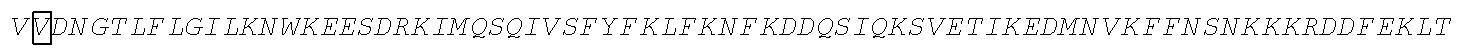

NYSVTDLNVQRKAIHELIQVMAELSPAAKTGKRKRSQMLFRGGGGSGGGGSGGGGSGGGGSQDPYVKEAENL

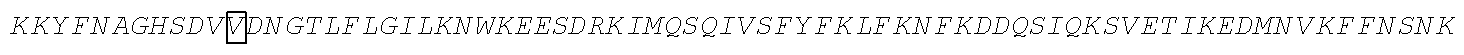

KKRDDFEKLTNYSVTDLNVQRKAIHELIQVMAELSPAAKTGKRKRSQMLFRGGGGGSGGGDKTHTCPPCPAP

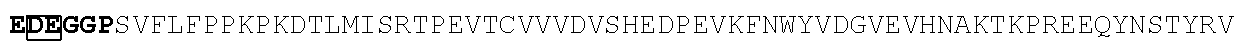

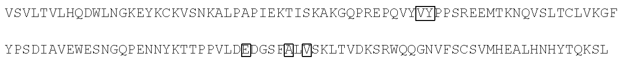

SLSPGK
SEQ ID NO: 193 (human CD155 extracellular domain-linker-single-chain mutant homodimer IFN-γ
(A23V/A23V)-linker-hinge-IgG1 Fc mutant2; CD155 extracellular domain is underlined; linker is bolded
and underlined; hinge is bolded; IFN-γ is italicized)
WPPPGTGDVVVQAPTQVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGELSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

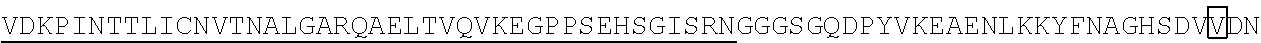

GTLFLGILKNWKEESDRKIMQSQIVSFYFKILFKNFKDDQSIQKSVETIKEDMNVKFFNSNKKKRDDFEKLTNYSVT
DLNVQRKAIHELIQVMAELSPAAKTGKRKRSQMLFRGGGGSGGGGSGGGGSGGGGSQDPYVKEAENLKKYF

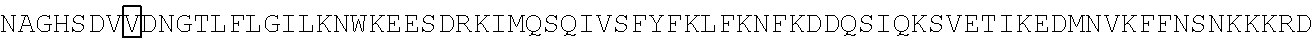

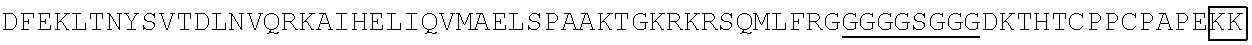

GGPSVFLFPPKPKDTLMISRTPEVTCVVVDVSHEDPEVKFNWYVDGVEVHNAKTKPREEQYNSTYRVVSV

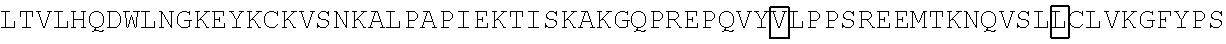

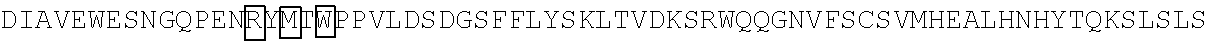

PGK
SEQ ID NO: 194 (linker; n is an integer of at least 1)
(G)ₙ
SEQ ID NO: 195 (linker; n is an integer of at least 1)
(GS)ₙ
SEQ ID NO: 196 (linker; n is an integer of at least 1)
(GGS)ₙ
SEQ ID NO: 197 (linker; n is an integer of at least 1)
(GGGS)ₙ
SEQ ID NO: 198 (linker; n is an integer of at least 1)
(GGS)ₙ(GGGS)ₙ
SEQ ID NO: 199 (linker; n is an integer of at least 1)
(GSGGS)ₙ
SEQ ID NO: 200 (linker; n is an integer of at least 1)
(GGSGS)ₙ
SEQ ID NO: 201 (linker; n is an integer of at least 1)
(GGGGS)n
SEQ ID NO: 202 (linker)
GG
SEQ ID NO: 203 (linker)
GSG
SEQ ID NO: 204 (linker)
GGSG
SEQ ID NO: 205 (linker)
GGSGG
SEQ ID NO: 206 (linker)
GSGGGGG
SEQ ID NO: 207 (linker)
GSGSG
SEQ ID NO: 208 (linker)
GSGGG
SEQ ID NO: 209 (linker)
GGGSG
SEQ ID NO: 210 (linker)
GSSSG
SEQ ID NO: 211 (linker)
GGSGGS
SEQ ID NO: 212 (linker)
SGGGGS
SEQ ID NO: 213 (linker)
GGGGS
SEQ ID NO: 214 (linker; n is an integer of at least 1)
(GA)ₙ
SEQ ID NO: 215 (linker)
GRAGGGGAGGGG
SEQ ID NO: 216 (linker)
GRAGGG
SEQ ID NO: 217 (linker)
GSGGGSGGGGSGGGGS
SEQ ID NO: 218 (linker)
GGGSGGGGSGGGGS
SEQ ID NO: 219 (linker)
GGGSGGSGGS
SEQ ID NO: 220 (linker)
GGSGGSGGSGGSGGG
SEQ ID NO: 221 (linker)
GGSGGSGGGGSGGGGS
SEQ ID NO: 222 (linker)
GGSGGSGGSGGSGGSGGS
SEQ ID NO: 223 (linker)
GGGGGGSGGGGSGGGGSA
SEQ ID NO: 224 (linker)
GSGGGSGGGGGGGGSGGGGS
SEQ ID NO: 225 (linker)
KTGGGSGGGS
SEQ ID NO: 226 (linker)
GGPGGGGSGGGSGGGGS
SEQ ID NO: 227 (linker)
GGGSGGGGSGGGGSGGGGS
SEQ ID NO: 228 (linker)
GGGGSGGGGSGGGGSGGGGSG
SEQ ID NO: 229 (linker)
GGGGSGGGGSGGGGS
SEQ ID NO: 230 (linker)
ASTKGP
SEQ ID NO: 231 (linker)
DKP
SEQ ID NO: 232 (linker)
DKPGS
SEQ ID NO: 233 (linker)
PGS
SEQ ID NO: 234 (linker)
GS
SEQ ID NO: 235 (linker)
DKPGSG
SEQ ID NO: 236 (linker)
PGSG
SEQ ID NO: 237 (linker)
DKPGSGS
SEQ ID NO: 238 (linker)
PGSGS
SEQ ID NO: 239 (linker)
GSGS
SEQ ID NO: 240 (linker)
DKPGSGGGGG
SEQ ID NO: 241 (linker)
PGSGGGGG
SEQ ID NO: 242 (linker)
P
SEQ ID NO: 243
GGGGSGGGSGGGG
SEQ ID NO: 244
GGGGSGGG
SEQ ID NO: 245
GGPGGGGSGGGSGGGG
SEQ ID NO: 246
GGGGSGGGSGGGGS
SEQ ID NO: 247 (human CD155 extracellular domain-linker- IL-2 mutant (L18R/Q22E/R38D/K43E/E61R)-
linker-hinge-IgG1 Fc mutant2; CD155 extracellular domain is underlined; linker is bolded and
underlined; hinge is bolded; IL-12 subunits are italicized)
WPPPGTGDVVVOAPTQVPGELGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGELSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

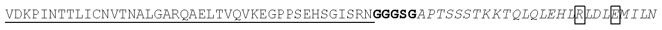

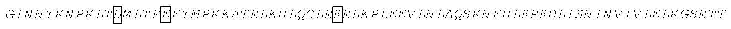

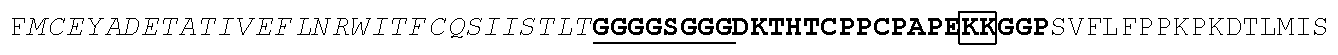

RTPEVTCVVVDVSHEDPEVKFNWYVDGVEVHNAKTKPREEQYNSTYRVVSVLTVLHQDWLNGKEYKCK

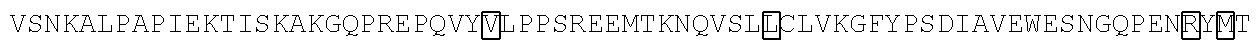

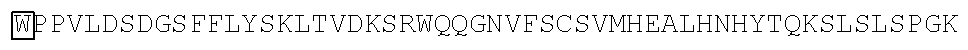

SEQ ID NO: 248 (buman CD155 extracellular domain-linker-IL-2 mutant (R38D/K43E/E61R/Q126T)-
linker-hinge-IgG1 Fc mutant2; CD155 extracellular domain is underlined; linker is bolded and
underlined; hinge is bolded; IL-12 subunits are italicized)
WPPPGTGDVVVQAPTQVPGFLGDSVTLPCYLQVPNMEVTHVSOLTWARHGESGSMAVFHQTQGPSYSES
KRLEFVAARLGAELRNASLRMFGLRVEDEGNYTCLFVTFPOGSRSVDIWLRVLAKPQNTAEVQKVQLTGE
PVPMARCVSTGGRPPAQITWHSDLGGMPNTSOVPGFLSGTVTVTSLWILVPSSQVDGKNVTCKVEHESFEK
PQLLTVNLTVYYPPEVSISGYDNNWYLGONEATLTCDARSNPEPTGYNWSTTMGPLPPFAVAQGAQLLIRP
VDKPINTTLICNVTNALGARQAELTVQVKEGPPSEHSGISRNGGGSGAPTSSSTKKTQLQLEHLLLDLQMILN

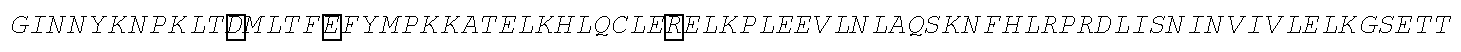

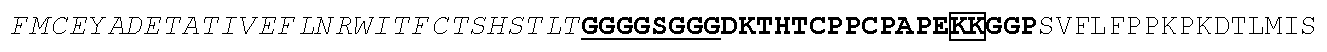

RTPEVTCVVVDVSHEDPEVKFNWYVDGVEVHNAKTKPREEQYNSTYRVVSVLTVLHQDWLNGKEYKCK

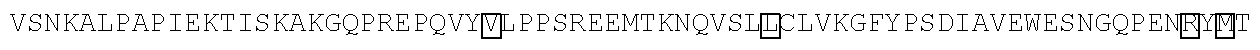

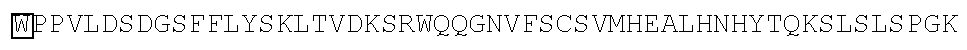

SEQ ID NO: 249 (human CD155 extracellular domain-linker-IL-2 mutant
(L18R/Q22E/R38D/K43E/E61R/Q126T)-linker-hinge-IgG1 Fe mutant2; CD 155 extracellular domain is
underlined; linker is bolded and underlined; hinge is bolded; IL-12 subunits are italicized)
WPPPGTGDVVVQAPTOVPGFLGDSVTLPCYLQVPNMEVTHVSQLTWARHGESGSMAVFHQTQGPSYSES
KRLEFVAARLGAELRNASLRMFGLRVEDEGNYTCLFVTFPQGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

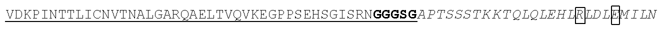

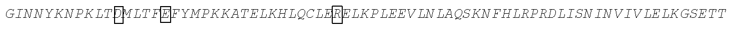

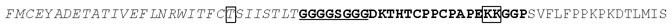

RTPEVTCVVVDVSHEDPEVKFNWYVDGVEVHNAKTKPREEQYNSTYRVVSVLTVLHQDWLNGKEYKCK

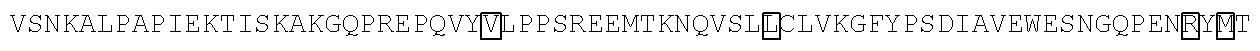

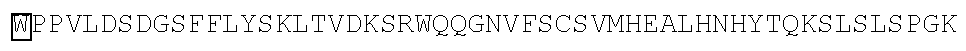

SEQ ID NO: 250 (haman CD155 extracellular domain-linker-IL-2 mutant
(L18R/Q22E/R38D/K43E/E61RQ126T/S130R)-linker-hinge-IgG1 Fe mutant2; CD155 extracellular domain is
underlined; linker is bolded and underlined; hinge is bolded; IL-12 subunits are italicized)
WPPPGTGDVVVQAPTQVPGFLGDSVILPCYLQVPNMEVTHVSQLTWARHGESGSMAVFHQTQGPSYSES
KRLEFVAARLGAELRNASLRMFGLRVEDEGNYTCLFVTFPOGSRSVDIWLRVLAKPQNTAEVQKVQLTGE
PVPMARCVSTGGRPPAQITWHSDLGGMPNTSQVPGFLSGTVTVTSLWILVPSSQVDGKNVTCKVEHESFEK
PQLLTVNLTVYYPPEVSISGYDNNWYLGQNEATLTCDARSNPEPTGYNWSTTMGPLPPFAVAQGAQLLIRP

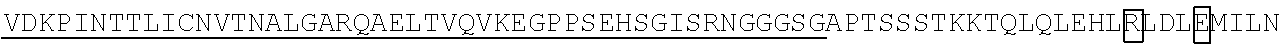

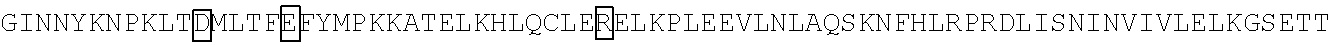

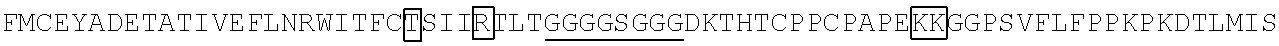

RTPEVTCVVVDVSHEDPEVKFNWYVDGVEVHNAKTKPREEQYNSTYRVVSVLTVLHQDWLNGKEYKCK

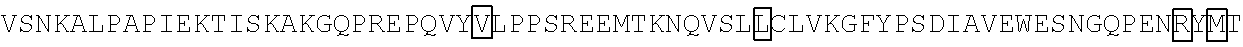

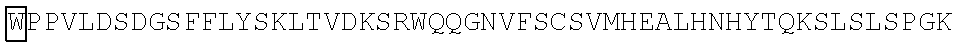

SEQ ID NO: 251 (single-chain ″wildtype″ IFN-γ bomodimer; linker is bolded; wildtype IFN-γ monomer is
italicized)
QDPYVKEAENLKKYFNAGHSDVADNGTLFLGILKNWKEESDRKIMQSQIVSFYFKLFKNEKDDQSIQKSVETIKE
DMNVKFFNSNKKKRDDFEKLTNYSVTDLNVORKAIHELIQVMAELSPAAKTGKRKRSOMLFRGGGGGGGGS
GGGGSGGGGSQDPYVKEAENLKKYFNAGHSDVADNGTLFLGILKNWKEESDRKIMQSQIVSFYFKLFKNFKDD
QSIQKSVETIKEDMNVKFFNSNKKKRDDFEKLINYSVTDLNVQRKAIHELIQVMAELSPAAKTGKRKRSQMLERG
SEQ ID NO: 252 (single-chain IFN-γ mutant A23V homodimer; linker is bolded; IFN-γ mutant monomer is
italicized)

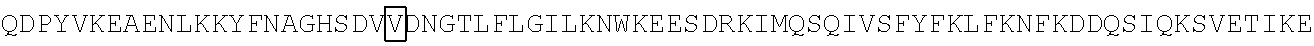

DMNVKFFNSNKKKRDDFEKLTNYSVTDLNVQRKAIHELIQVMAELSPAAKTGKRKRSQMLFRGGGGSGGGGS

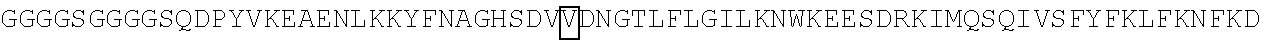

DQSIQKSVETIKEDMNVKFFNSNKKKRDDFEKLTNYSVTDLNVQRKAIHELIQVMAELSPAAKTGKRKRSQMLFR
G
SEQ ID NO: 253 (single-chain ″wildtype″ IL-12 heterodimer IL-12B (wt p40)-linker-IL-12A (wt p35);
linker is bolded)
IWELKKDVYVVELDWYPDAPGEMVVLTCDTPEEDGITWTLDOSSEVLGSGKTLTIQVKEFGDAGQYTCHKGGEV
LSHSLLLLHKKEDGIWSTDILKDQKEPKNKTFLRCEAKNYSGRFTCWWLTTISTDLTFSVKSSRGSSDPOGVTCGA
ATLSAERVRGDNKEYEYSVECQEDSACPAAEESLPIEVMVDAVHKLKYENYTSSFFIRDIIKPDPPKNLOLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGSGGGGSRNLPVATPDPGMFPCLHHSQNLLRAVSNMLQKARQTLEFYPCTSEEIDHEDITKD
KTSTVEACLPLELTKNESCLNSRETSFITNGSCLASRKTSFMMALCLSSIYEDLKMYQVEFKTMNAKLLMDPKRQIF
LDQNMLAVIDELMQALNFNSETVPOKSSLEEPDFYKTKIKLCILLHAFRIRAVTIDRVMSYLNAS
SEQ ID NO: 254 (single-chain IL-12 mutant heterodimer IL-12B (p40 E59A/F60A)-linker-IL-12A (wt p35);
linker is bolded; IL-12 subunits are italicized)

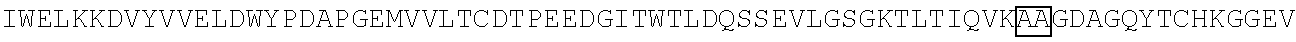

LSHSLLLLHKKEDGIWSTDILKDOKEPKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYENYTSSFFIRDIIKPDPPKNLQLKPLKNS
RQVEVSWEYPDTWSTPHSYFSLTFCVQVQGKSKREKKDRVFTDKTSATVICRKNASISVRAQDRYYSSSWSEWASV
PCSGGGGSGGGSGGGGSRNLPVATPDPGMFPCLHHSONLLRAVSNMLOKAROTLEFYPCTSEEIDHEDITKD
KTSTVEACLPLELTKNESCLNSRETSFITNGSCLASRKTSFMMALCLSSIYEDLKMYQVEFKTMNAKLLMDPKRQIF
LDQNMLAVIDELMQALNENSETVPQKSSLEEPDFYKTKIKLCILLHAFRIRAVTIDRVMSYLNAS

1-58. (canceled)

59. An immunomodulatory molecule comprising a first binding domain specifically recognizing a first target molecule and a second binding domain specifically recognizing a second target molecule, wherein the first binding domain upon binding to the first target molecule up-regulates an immune response, and wherein the second binding domain upon binding to the second target molecule down-regulates the immune response, wherein the first binding domain is an immunostimulatory cytokine or variant thereof and the second binding domain is an agonist of an inhibitory checkpoint molecule.

60. The immunomodulatory molecule of claim 59, wherein the immunostimulatory cytokine or variant thereof is selected from the group consisting of IL-1, IL-2, IL-3, IL-4, IL-5, IL-6, IL-7, IL-8, IL-9, IL-12, IL-15, IL-17, IL-18, IL-21, IL-22, IL-23, IL-27, IFN-α, IFN-β, IFN-γ, TNF-α, erythropoietin, thrombopoietin, G-CSF, M-CSF, SCF, and GM-CSF.

61. The immunomodulatory molecule of claim 60, wherein the immunostimulatory cytokine or variant thereof is IL-12 or variant thereof.

62. The immunomodulatory molecule of claim 61, wherein the IL-12 variant comprises one or more mutations within the p40 subunit at a position selected from the group consisting of E45, Q56, V57, K58, E59, F60, G61, D62, A63, G64, Q65, and C177 relative to a wildtype p40 subunit.

63. The immunomodulatory molecule of claim 62, wherein the IL-12 variant comprises an E59A mutation, an F60A mutation, an F60D mutation, or an E59A/F60A mutation within the p40 subunit relative to a wildtype p40 subunit.

64. The immunomodulatory molecule of claim 63, wherein the p40 subunit of the IL-12 variant comprises the amino acid sequence of any of SEQ ID NOs: 63-66 and 140.

65. The immunomodulatory molecule of claim 60, wherein the immunostimulatory cytokine or variant thereof is IL-2 or variant thereof.

66. The immunomodulatory molecule of claim 65, wherein the IL-2 variant comprises one or more mutations at a position selected from the group consisting of L18, Q22, F24, K35, R38, F42, K43, E61, P65, Q126, and S130 relative to a wildtype IL-2 (SEQ ID NO: 25).

67. The immunomodulatory molecule of claim 66, wherein the IL-2 variant comprises an R38D/K43E/E61R mutation, an L18R/Q22E/R38D/K43E/E61R mutation, an R38D/K43E/E61R/Q126T, an L18R/Q22E/R38D/K43E/E61R/Q126T mutation, or an L18R/Q22E/R38D/K43E/E61R/Q126T/S130R mutation relative to wildtype IL-2 (SEQ ID NO: 25).

68. The immunomodulatory molecule of claim 59, wherein the inhibitory checkpoint molecule is selected from the group consisting of PD-1, PD-L1, PD-L2, CTLA-4, LAG-3, TIM-3, HHLA2, CD47, CXCR4, CD160, CD73, BLTA, B7-H4, TIGIT, Siglec7, Siglec9, and VISTA.

69. The immunomodulatory molecule of claim 59, wherein the agonist of the inhibitory checkpoint molecule is a ligand or an antibody or antigen-binding fragment thereof.

70. The immunomodulatory molecule of claim 69,
(i) wherein the inhibitory checkpoint molecule is PD-1, and wherein the agonist of the inhibitory checkpoint molecule is PD-L1, PD-L2, or variant thereof;
(ii) wherein the inhibitory checkpoint molecule is TIGIT, and wherein the agonist of the inhibitory checkpoint molecule is CD 112, CD155, or variant thereof;
(iii) wherein the inhibitory checkpoint molecule is LAG-3, and wherein the agonist of the inhibitory checkpoint molecule is MHC II, LSECtin, or variant thereof;
(iv) wherein the inhibitory checkpoint molecule is TIM-3, and wherein the agonist of the inhibitory checkpoint molecule is Galectin-9, Caecam-1, HMGB-1, phosphatidylserine, or variant thereof; or
(v) wherein the inhibitory checkpoint molecule is CTLA-4, and wherein the agonist of the inhibitory checkpoint molecule is CD80, CD86, or variant thereof.

71. The immunomodulatory molecule of claim 59, wherein the immunomodulatory molecule comprises:
(i) an antigen-binding protein comprising an antigen-binding polypeptide and the first binding domain, wherein the antigen-binding polypeptide comprises from N-terminus to C-terminus: the second binding domain or portion thereof, a hinge region, and an Fc domain subunit or portion thereof, and wherein the first binding domain is positioned at the hinge region;
(ii) an antigen-binding protein comprising an antigen-binding polypeptide, wherein the antigen-binding polypeptide comprises from N′ to C′: the first binding domain or portion thereof, the second binding domain or portion thereof, an optional hinge region, and an Fc domain subunit or portion thereof;
(iii) an antigen-binding protein comprising an antigen-binding polypeptide, wherein the antigen-binding polypeptide comprises from N-terminus to C-terminus: the second binding domain or portion thereof, an optional hinge region, an Fc domain subunit or portion thereof, and the first binding domain or portion thereof;
(iv) an antigen-binding protein comprising an antigen-binding polypeptide, wherein the antigen-binding polypeptide comprises from N-terminus to C-terminus: the second binding domain or portion thereof, an optional hinge region, an Fc domain subunit or portion thereof, and the first binding domain or portion thereof.

72. The immunomodulatory molecule of claim 59, wherein the immunomodulatory molecule comprises:
(i) a first antigen-binding polypeptide comprising from N-terminus to C-terminus: a first PD-L2 or PD-L1 or variant thereof, a second PD-L2 or PD-L1 or variant thereof, a p35 subunit and a p40 subunit of an IL-12 or variant thereof positioned in tandem at a first hinge region, and a first subunit of an Fc domain or portion thereof; a second antigen-binding polypeptide comprising from N-terminus to C-terminus: a VH, an optional CH1, a second hinge region, and a second subunit of the Fc domain or portion thereof; and a third antigen-binding polypeptide comprising from N-terminus to C-terminus: a VL, and an optional CL; wherein the VH and the VL and optionally the CH1 and the CL form a third binding domain specifically recognizing a third target molecule;
(ii) a first antigen-binding polypeptide comprising from N-terminus to C-terminus: a first VH, an optional first CH1, a p35 subunit and a p40 subunit of an IL-12 or variant thereof positioned in tandem at a first hinge region, and a first subunit of an Fc domain or portion thereof; a second antigen-binding polypeptide comprising from N-terminus to C-terminus: a second VH, an optional second CH1, a second hinge region, and a second subunit of the Fc domain or portion thereof; a third antigen-binding polypeptide comprising from N-terminus to C-terminus: a first VL, and an optional first CL; and a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule;
(iii) a first antigen-binding polypeptide comprising from N-terminus to C-terminus: a first PD-L2 or PD-L1 or variant thereof, a p35 subunit and a p40 subunit of an IL-12 or variant thereof positioned in tandem at a first hinge region, and a first subunit of an Fc domain or portion thereof; and a second antigen-binding polypeptide comprising from N-terminus to C-terminus: a second PD-L2 or PD-L1 or variant thereof, a second hinge region, and a second subunit of an Fc domain or portion thereof;
(iv) a first antigen-binding polypeptide comprising from N-terminus to C-terminus: a first PD-L2 or PD-L1 or variant thereof, a second PD-L2 or PD-L1 or variant thereof, a p35 subunit and a p40 subunit of an IL-12 or variant thereof positioned in tandem at a first hinge region, and a first subunit of an Fc domain or portion thereof; and a second antigen-binding polypeptide comprising from N-terminus to C-terminus: a third PD-L2 or PD-L1 or variant thereof, a fourth PD-L2 or PD-L1 or variant thereof, a second hinge region, and a second subunit of the Fc domain or portion thereof;
(v) a first antigen-binding polypeptide comprising from N-terminus to C-terminus: a first PD-L2 or PD-L1 or variant thereof, a p35 subunit of an IL-12 or variant thereof positioned at a first hinge region, and a first subunit of an Fc domain or portion thereof; and a second antigen-binding polypeptide comprising from N-terminus to C-terminus: a second PD-L2 or PD-L1 or variant thereof, a p40 subunit of an IL-12 or variant thereof positioned at a second hinge region, and a second subunit of the Fc domain or portion thereof;
(vi) a first antigen-binding polypeptide comprising from N-terminus to C-terminus: a p35 subunit or a p40 subunit of an IL-12 or variant thereof positioned at a first hinge region, and a first subunit of an Fc domain or portion thereof; and a second antigen-binding polypeptide comprising from N-terminus to C-terminus: a first PD-L2 or PD-L1 or variant thereof, a second PD-L2 or PD-L1 or variant thereof, a p40 subunit or a p35 subunit of an IL-12 or variant thereof positioned at a second hinge region, and a second subunit of the Fc domain or portion thereof;
(vii) a first antigen-binding polypeptide comprising from N-terminus to C-terminus: a first VH, an optional first CH1, a p35 subunit or a p40 subunit of an IL-12 or variant thereof positioned at a first hinge region, and a first subunit of an Fc domain or portion thereof; a second antigen-binding polypeptide comprising from N-terminus to C-terminus: a second VH, an optional second CH1, a p40 subunit or a p35 subunit of an IL-12 or variant thereof positioned at a second hinge region, and a second subunit of the Fc domain or portion thereof; a third antigen-binding polypeptide comprising from N-terminus to C-terminus: a first VL, and an optional first CL; and a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule;
(viii) a first antigen-binding polypeptide comprising from N-terminus to C-terminus: a first PD-L2 or PD-L1 or variant thereof, an IL-2 or variant thereof, a first hinge region, a first subunit of an Fc domain or portion thereof; and a second antigen-binding polypeptide comprising from N-terminus to C-terminus: a second PD-L2 or PD-L1 or variant thereof, a p35 subunit and a p40 subunit of an IL-12 or variant thereof connected in tandem, a second hinge region, and a second subunit of the Fc domain or portion thereof;
(ix) a first antigen-binding polypeptide comprising from N-terminus to C-terminus: a third binding domain, an IL-2 or variant thereof, a first hinge region, a first subunit of an Fc domain or portion thereof; and ii) a second antigen-binding polypeptide comprising from N-terminus to C-terminus: a PD-L2 or PD-L1 or variant thereof, a p35 subunit and a p40 subunit of an IL-12 or variant thereof connected in tandem, a second hinge region, and a second subunit of the Fc domain or portion thereof, wherein the third binding domain is a Fab or sdAb specifically recognizing TIGIT, TIM3, LAG3, CTLA4, CD16A, HER2, Nectin-4, Trop2, or CLDN18.2;
(x) a first antigen-binding polypeptide comprising from N-terminus to C-terminus: a first VH, an optional first CH1, a first hinge region, and a first subunit of an Fc domain or portion thereof; a second antigen-binding polypeptide comprising from N-terminus to C-terminus: a second VH, an optional second CH1, a second hinge region, and a second subunit of the Fc domain or portion thereof; a third antigen-binding polypeptide comprising from N-terminus to C-terminus: a p35 subunit and a p40 subunit of an IL-12 or variant thereof fused in tandem, a first VL, and an optional first CL; and a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, and wherein the second VH and the second VL and optionally the second CH1 and the second CL form a third binding domain specifically recognizing a third target molecule; or
(xi) a first antigen-binding polypeptide comprising from N-terminus to C-terminus: a VH, an optional CH1, a first hinge region, and a first subunit of an Fc domain or portion thereof; a second antigen-binding polypeptide comprising from N-terminus to C-terminus: a first PD-L2 or PD-L1 or variant thereof, a second PD-L2 or PD-L1 or variant thereof, a second hinge region, and a second subunit of the Fc domain or portion thereof; and a third antigen-binding polypeptide comprising from N-terminus to C-terminus: a p35 subunit and a p40 subunit of an IL-12 or variant thereof fused in tandem, a VL, and an optional CL, wherein the VH and the VL and optionally the CH1 and the CL form the second binding domain which is an agonist antigen-binding fragment specifically recognizing PD-1.

73. The immunomodulatory molecule of claim 59, wherein the immunomodulatory molecule comprises:
(i) a first antigen-binding polypeptide comprising from N-terminus to C-terminus: a first PD-L2 or PD-L1 or variant thereof, a p35 subunit of an IL-12 or variant thereof positioned at a first hinge region, and a first subunit of an Fc domain or portion thereof; and a second antigen-binding polypeptide comprising from N-terminus to C-terminus: a second PD-L2 or PD-L1 or variant thereof, a p40 subunit of an IL-12 or variant thereof positioned at a second hinge region, and a second subunit of the Fc domain or portion thereof; or
(ii) a first antigen-binding polypeptide comprising from N-terminus to C-terminus: a first VH, an optional first CH1, a p35 subunit or a p40 subunit of an IL-12 or variant thereof positioned at a first hinge region, and a first subunit of an Fc domain or portion thereof; a second antigen-binding polypeptide comprising from N-terminus to C-terminus: a second VH, an optional second CH1, a p40 subunit or a p35 subunit of an IL-12 or variant thereof positioned at a second hinge region, and a second subunit of the Fc domain or portion thereof; a third antigen-binding polypeptide comprising from N-terminus to C-terminus: a first VL, and an optional first CL; and a fourth antigen-binding polypeptide comprising from N-terminus to C-terminus: a second VL, and an optional second CL, wherein the first VH and the first VL and optionally the first CH1 and the first CL form the second binding domain which is an agonist antigen-binding fragment specifically recognizing PD-1, wherein the second VH and the second VL and optionally the second CH1 and the second CL form a third binding domain specifically recognizing a third target molecule.

74. A pharmaceutical composition comprising the immunomodulatory molecule of claim 59, and optionally a pharmaceutical acceptable carrier.

75. A method of treating a disease or disorder in an individual, comprising administering to the individual an effective amount of the immunomodulatory molecule of claim 59.

76. An isolated nucleic acid or a vector encoding the immunomodulatory molecule of claim 59.

77. A host cell comprising the isolated nucleic acid or the vector of claim 76.

78. A method of producing an immunomodulatory molecule, comprising:
(a) culturing the host cell of claim 77 under a condition effective to express the encoded immunomodulatory molecule; and
(b) obtaining the expressed immunomodulatory molecule from said host cell.
